(19) United States
(12) Patent Application Publication
(10) Pub. No.: US 2023/0140983 A1
(43) Pub. Date: May 11, 2023

(54) PYRIDAZINE DERIVATIVES FOR MODULATING NUCLEIC ACID SPLICING

(71) Applicant: REMIX THERAPEUTICS INC., Cambridge, MA (US)

(72) Inventors: Dominic Reynolds, Stoneham, MA (US); Michael W. Seiler, Belmont, MA (US); Anant A. Agrawal, Waltham, MA (US); Frederic Vaillancourt, Newton, MA (US); Allen T. Hopper, Lexington, MA (US); Peter Smith, Arlington, MA (US)

(21) Appl. No.: 17/802,702

(22) PCT Filed: Feb. 28, 2021
(86) PCT No.: PCT/US2021/020173

§ 371 Date: Aug. 26, 2022

Related U.S. Application Data
(60) Provisional application No. 62/983,537, filed on Feb. 28, 2020.
(60) Provisional application No. 63/007,134, filed on Apr. 8, 2020.
(60) Provisional application No. 63/040,474, filed on Jun. 17, 2020.
(60) Provisional application No. 63/072,781, filed on Aug. 31, 2020.
(60) Provisional application No. 63/126,491, filed on Dec. 16, 2020.

(51) Int. Cl. C07D 471/10; C07D 401/14; C07D 471/08; C07D 409/14; C07D 417/04; C07D 471/04; C07D 487/04; C07D 487/08

(52) U.S. Cl. CPC C07D 471/10; C07D 401/14; C07D 471/08; C07D 409/14; C07D 417/04; C07D 471/04; C07D 487/04; C07D 487/08; C07B 2200/05

(57) ABSTRACT

The present disclosure features compounds and related compositions that, inter alia, modulate nucleic acid splicing, e.g., splicing of a pre-mRNA, as well as methods of use thereof.

### CLAIM OF PRIORITY

This application claims priority to U.S. Application No. 62/983,537, filed Feb. 28, 2020; U.S. Application No. 63/007,134, filed Apr. 8, 2020; U.S. Application No. 63/040,474, filed Jun. 17, 2020; U.S. Application No. 63/072,781, filed Aug. 31, 2020; and U.S. Application No. 63/126,491, filed Dec. 16, 2020. The disclosure of each of the foregoing applications is incorporated herein by reference in its entirety.

### BACKGROUND

Alternative splicing is a major source of protein diversity in higher eukaryotes and is frequently regulated in a tissue-specific or development stage-specific manner. Disease associated alternative splicing patterns in pre-mRNAs are often mapped to changes in splice site signals or sequence motifs and regulatory splicing factors (Faustino and Cooper (2003), Genes Dev 17 (4):419-37). Current therapies to modulate RNA expression involve oligonucleotide targeting and gene therapy; however, each of these modalities exhibit unique challenges as currently presented. As such, there is a need for new technologies to modulate RNA expression, including the development of small molecule compounds that target splicing.

### SUMMARY

The present disclosure features compounds and related compositions that, inter alia, modulate nucleic acid splicing, e.g., splicing of a pre-mRNA, as well as methods of use thereof.

In an embodiment, the compounds described herein are compounds of Formula (I) (e.g., a compound of Formulas (I-a), (I-b), (I-c), (I-d), (I-e), (I-f), (I-g), or (I-h)) and pharmaceutically acceptable salts, solvates, hydrates, tautomers, or stereoisomers thereof. The present disclosure additionally provides methods of using the compounds of the invention (e.g., compounds of Formulas (I), (I-a), (I-b), (I-c), (I-d), (I-e), (I-f), (I-g), or (I-h), and pharmaceutically acceptable salts, solvates, hydrates, tautomers, stereoisomers thereof), and compositions thereof, e.g., to target, and in embodiments bind or form a complex with, a nucleic acid (e.g., a pre-mRNA or nucleic acid component of a small nuclear ribonucleoprotein (snRNP) or spliceosome), a protein (e.g., a protein component of an snRNP or spliceosome, e.g., a member of the splicing machinery, e.g., one or more of the U1, U2, U4, U5, U6, U11, U12, U4atac, U6atac snRNPs), or a combination thereof. In another aspect, the compounds described herein may be used to alter the composition or structure of a nucleic acid (e.g., a pre-mRNA or mRNA (e.g., a pre-mRNA and the mRNA which arises from the pre-mRNA), e.g., by increasing or decreasing splicing at a splice site. In some embodiments, increasing or decreasing splicing results in modulating the level of a gene product (e.g., an RNA or protein) produced.

In another aspect, the compounds described herein may be used for the prevention and/or treatment of a disease, disorder, or condition, e.g., a disease, disorder or condition associated with splicing, e.g., alternative splicing. In some embodiments, the compounds described herein (e.g., compounds of Formulas (I), (I-a), (I-b), (I-c), (I-d), (I-e), (I-f), (I-g), or (I-h), and pharmaceutically acceptable salts, solvates, hydrates, tautomers, stereoisomers thereof) and compositions thereof are used for the prevention and/or treatment of a proliferative disease, disorder, or condition (e.g., a disease, disorder, or condition characterized by unwanted cell proliferation, e.g., a cancer or a benign neoplasm) in a subject. In some embodiments, the compounds described herein (e.g., compounds of Formulas (I), (I-a), (I-b), (I-c), (I-d), (I-e), (I-f), (I-g), or (I-h), and pharmaceutically acceptable salts, solvates, hydrates, tautomers, stereoisomers thereof) and compositions thereof are used for the prevention and/or treatment of a non-proliferative disease, disorder, or condition. In some embodiments, the compounds described herein (e.g., compounds of Formulas (I), (I-a), (I-b), (I-c), (I-d), (I-e), (I-f), (I-g), or (I-h), and pharmaceutically acceptable salts, solvates, hydrates, tautomers, stereoisomers thereof) and compositions thereof are used for the prevention and/or treatment of a neurological disease or disorder, an autoimmune disease or disorder, immunodeficiency disease or disorder, a lysosomal storage disease or disorder, a cardiovascular disease or disorder, a metabolic disease or disorder, a respiratory disease or disorder, a renal disease or disorder, or an infectious disease in a subject.

In one aspect, the present disclosure provides compounds of Formula (I):

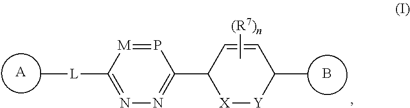

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —C(O)—, —N(R³)—, —S(O)ₓ—, —N(R³)C(O)—, or —C(O)N(R³)—, wherein each alkylene and heteroalkylene is optionally substituted with one or more R⁴; M and P are each independently C(R²) or N; X and Y are each independently C, C(R^{5a}) C(R^{5a})(R^{5b}), N, or N(R^{5c}), wherein the bond between X and Y may be a single or double bond as valency permits, and wherein X and Y may not both be C(R^{5a})(R^{5b}); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, halo, cyano, C₁-C₆-alkyl, C₂-C₆-alkenyl, C₂-C₆-alkynyl, or —OR^{A}; each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R¹²; each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R^{5a} is hydrogen, C₁-C₆-alkyl, or —ORF; R^{5b} is hydrogen or C₁-C₆-alkyl; or R^{5a} and R^{5b}, together with the carbon atom to which they are attached, form an oxo group; each R^{5c} is hydrogen, C₁-C₆-alkyl, C₁-C₆-haloalkyl, or C(O)R^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹; or two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; RF is hydrogen or C₁-C₆ alkyl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; n is 0, 1, 2, 3, or 4; and x is 0, 1, or 2.

In another aspect, the present invention provides pharmaceutical compositions comprising a compound of Formula (I) (e.g., a compound of Formulas (I-a), (I-b), (I-c), (I-d), (I-e), (I-f), (I-g), or (I-h)), or a pharmaceutically acceptable salt, solvate, hydrate, tautomer, or stereoisomer thereof, and optionally a pharmaceutically acceptable excipient. In an embodiment, the pharmaceutical compositions described herein include an effective amount (e.g., a therapeutically effective amount) of a compound of Formula (I) (e.g., a compound of Formulas (I), (I-a), (I-b), (I-c), (I-d), (I-e), (I-f), (I-g), or (I-h)), or a pharmaceutically acceptable salt, solvate, hydrate, tautomer, or stereoisomer thereof.

In another aspect, the present disclosure provides methods for modulating splicing, e.g., splicing of a nucleic acid (e.g., a DNA or RNA, e.g., a pre-mRNA) with a compound of Formula (I) (e.g., a compound of Formulas (I), (I-a), (I-b), (I-c), (I-d), (I-e), (I-f), (I-g), or (I-h)) or a pharmaceutically acceptable salt, solvate, hydrate, tautomer, or stereoisomer thereof. In another aspect, the present disclosure provides compositions for use in modulating splicing, e.g., splicing of a nucleic acid (e.g., a DNA or RNA, e.g., a pre-mRNA) with a compound of Formula (I) (e.g., a compound of Formulas (I), (I-a), (I-b), (I-c), (I-d), (I-e), (I-f), (I-g), or (I-h)) or a pharmaceutically acceptable salt, solvate, hydrate, tautomer, or stereoisomer thereof. Modulation of splicing may comprise impacting any step involved in splicing and may include an event upstream or downstream of a splicing event. For example, in some embodiments, the compound of Formula (I) binds to a target, e.g., a target nucleic acid (e.g., DNA or RNA, e.g., a precursor RNA, e.g., a pre-mRNA), a target protein, or combination thereof (e.g., an snRNP and a pre-mRNA). A target may include a splice site in a pre-mRNA or a component of the splicing machinery, such as the U1 snRNP. In some embodiments, the compound of Formula (I) alters a target nucleic acid (e.g., DNA or RNA, e.g., a precursor RNA, e.g., a pre-mRNA), target protein, or combination thereof. In some embodiments, the compound of Formula (I) increases or decreases splicing at a splice site on a target nucleic acid (e.g., an RNA, e.g., a precursor RNA, e.g., a pre-mRNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I), e.g., in a healthy or diseased cell or tissue). In some embodiments, the presence of a compound of Formula (I) results an increase or decrease of transcription of a target nucleic acid (e.g., an RNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I), e.g., in a healthy or diseased cell or tissue).

In another aspect, the present disclosure provides methods for preventing and/or treating a disease, disorder, or condition in a subject by administering a compound of Formula (I) (e.g., a compound of Formulas (I), (I-a), (I-b), (I-c), (I-d), (I-e), (I-f), (I-g), or (I-h)) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides methods of down-regulating the expression of (e.g., the level of or the rate of production of) a target protein with a compound of Formula (I) (e.g., a compound of Formulas (I), (I-a), (I-b), (I-c), (I-d), (I-e), (I-f), (I-g), or (I-h)) or a pharmaceutically acceptable salt, solvate, hydrate, tautomer, or stereoisomer thereof in a biological sample or subject. In another aspect, the present disclosure provides methods of up-regulating the expression of (e.g., the level of or the rate of production of) a target protein with a compound of Formula (I) (e.g., a compound of Formulas (I), (I-a), (I-b), (I-c), (I-d), (I-e), (I-f), (I-g), or (I-h)) or a pharmaceutically acceptable salt, solvate, hydrate, tautomer, or stereoisomer thereof in a biological sample or subject. In another aspect, the present disclosure provides methods of altering the isoform of a target protein with a compound of Formula (I) (e.g., a compound of Formulas (I), (I-a), (I-b), (I-c), (I-d), (I-e), (I-f), (I-g), or (I-h))) or a pharmaceutically acceptable salt, solvate, hydrate, tautomer, or stereoisomer thereof in a biological sample or subject. Another aspect of the disclosure relates to methods of inhibiting the activity of a target protein in a biological sample or subject. In some embodiments, administration of a compound of Formula (I) to a biological sample, a cell, or a subject comprises inhibition of cell growth or induction of cell death.

In another aspect, the present disclosure provides compositions for use in preventing and/or treating a disease, disorder, or condition in a subject by administering a compound of Formula (I) (e.g., a compound of Formulas (I), (I-a), (I-b), (I-c), (I-d), (I-e), (I-f), (I-g), or (I-h)) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides compositions for use in treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides compositions for use in down-regulating the expression of (e.g., the level of or the rate of production of) a target protein with a compound of Formula (I) (e.g., a compound of Formulas (I), (I-a), (I-b), (I-c), (I-d), (I-e), (I-f), (I-g), or (I-h)) or a pharmaceutically acceptable salt, solvate, hydrate, tautomer, or stereoisomer thereof in a biological sample or subject. In another aspect, the present disclosure provides compositions for use in up-regulating the expression of (e.g., the level of or the rate of production of) a target protein with a compound of Formula (I) (e.g., a compound of Formulas (I), (I-a), (I-b), (I-c), (I-d), (I-e), (I-f), (I-g), or (I-h)) or a pharmaceutically acceptable salt, solvate, hydrate, tautomer, or stereoisomer thereof in a biological sample or subject. In another aspect, the present disclosure provides compositions for use in altering the isoform of a target protein with a compound of Formula (I) (e.g., a compound of Formulas (I), (I-a), (I-b), (I-c), (I-d), (I-e), (I-f), (I-g), or (I-h))) or a pharmaceutically acceptable salt, solvate, hydrate, tautomer, or stereoisomer thereof in a biological sample or subject. Another aspect of the disclosure relates to compositions for use in inhibiting the activity of a target protein in a biological sample or subject. In some embodiments, administration of a compound of Formula (I) to a biological sample, a cell, or a subject comprises inhibition of cell growth or induction of cell death.

In another aspect, the present disclosure features kits comprising a container with a compound of Formula (I) (e.g., a compound of Formulas (I), (I-a), (I-b), (I-c), (I-d), (I-e), (I-f), (I-g), or (I-h)), or a pharmaceutically acceptable salt, solvate, hydrate, tautomer, stereoisomer thereof, or a pharmaceutical composition thereof. In certain embodiments, the kits described herein further include instructions for administering the compound of Formula (I) or the pharmaceutically acceptable salt, solvate, hydrate, tautomer, stereoisomer thereof, or the pharmaceutical composition thereof.

In any and all aspects of the present disclosure, in some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein other than a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, and WO 2019/199972. In some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, and WO 2019/199972, each of which is incorporated herein by reference in its entirety.

The details of one or more embodiments of the invention are set forth herein. Other features, objects, and advantages of the invention will be apparent from the Detailed Description, the Examples, and the Claims.

### DETAILED DESCRIPTION

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—Cl₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula —C(O)₂R′— may represent both —C(O)₂R′- and —R′C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substitutent having two oxygen atoms bound to a nitrogen atom, e.g., —NO₂.

As used herein, the term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring. In an embodiment, a nucleobase may be chemically modified, for example, with an alkyl (e.g., methyl), halo, —O-alkyl, or other modification.

As used herein, the term “nucleic acid” refers to deoxyribonucleic acids (DNA) or ribonucleic acids (RNA) and polymers thereof in either single- or double-stranded form. The term “nucleic acid” includes a gene, cDNA, pre-mRNA, or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated.

As used herein, “oxo” refers to a carbonyl, i.e., —C(O)—.

The symbol “ [symbol] ” as used herein in relation to a compound of Formula (I) refers to an attachment point to another moiety or functional group within the compound.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

The compounds provided herein may exist in one or more particular geometric, optical, enantiomeric, diasteriomeric, epimeric, stereoisomeric, tautomeric, conformational, or anomeric forms, including but not limited to: cis- and trans-forms; E- and Z-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising an enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising an enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound.

In some embodiments, a diastereomerically pure compound can be present with other active or inactive ingredients. For example, a pharmaceutical composition comprising a diastereometerically pure exo compound can comprise, for example, about 90% excipient and about 10% diastereometerically pure exo compound. In certain embodiments, the diastereometerically pure exo compound in such compositions can, for example, comprise, at least about 95% by weight exo compound and at most about 5% by weight endo compound, by total weight of the compound. For example, a pharmaceutical composition comprising a diastereometerically pure endo compound can comprise, for example, about 90% excipient and about 10% diastereometerically pure endo compound. In certain embodiments, the diastereometerically pure endo compound in such compositions can, for example, comprise, at least about 95% by weight endo compound and at most about 5% by weight exo compound, by total weight of the compound.

In some embodiments, an isomerically pure compound can be present with other active or inactive ingredients. For example, a pharmaceutical composition comprising a isomerically pure exo compound can comprise, for example, about 90% excipient and about 10% isomerically pure exo compound. In certain embodiments, the isomerically pure exo compound in such compositions can, for example, comprise, at least about 95% by weight exo compound and at most about 5% by weight endo compound, by total weight of the compound. For example, a pharmaceutical composition comprising an isomerically pure endo compound can comprise, for example, about 90% excipient and about 10% isomerically pure endo compound. In certain embodiments, the isomerically pure endo compound in such compositions can, for example, comprise, at least about 95% by weight endo compound and at most about 5% by weight exo compound, by total weight of the compound.

In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

In addition to salt forms, the present disclosure provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R.0.5 H₂O)), and polyhydrates (x is a number greater than 1, e.g., dihydrates (R.2 H₂O) and hexahydrates (R.6 H₂O)).

The term “tautomer” refers to compounds that are interchangeable forms of a particular compound structure, and that vary in the displacement of hydrogen atoms and electrons. Thus, two structures may be in equilibrium through the movement of R electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### Other Definitions

The following definitions are more general terms used throughout the present disclosure.

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

An “effective amount” of a compound of Formula (I) refers to an amount sufficient to elicit the desired biological response, i.e., treating the condition. As will be appreciated by those of ordinary skill in this art, the effective amount of a compound of Formula (I) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a compound described herein (e.g., a compound of Formula (I)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a compound described herein (e.g., a compound of Formula (I)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

A “proliferative disease” refers to a disease that occurs due to abnormal extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); 4) the pathological angiogenesis as in proliferative retinopathy and tumor metastasis; or 5) evasion of host immune surveillance and elimination of neoplastic cells. Exemplary proliferative diseases include cancers (i.e., “malignant neoplasms”), benign neoplasms, and angiogenesis.

A “non-proliferative disease” refers to a disease that does not primarily extend through the abnormal multiplication of cells. A non-proliferative disease may be associated with any cell type or tissue type in a subject. Exemplary non-proliferative diseases include neurological diseases or disorders (e.g., a repeat expansion disease); autoimmune disease or disorders; immunodeficiency diseases or disorders; lysosomal storage diseases or disorders; inflammatory diseases or disorders; cardiovascular conditions, diseases, or disorders; metabolic diseases or disorders; respiratory conditions, diseases, or disorders; renal diseases or disorders; and infectious diseases.

### Compounds

The present disclosure features a compound of Formula (I):

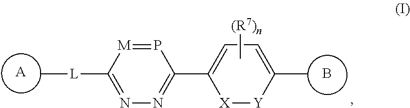

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —C(O)—, —N(R³)—, —S(O)ₓ—, —N(R³)C(O)—, or —C(O)N(R³)—, wherein each alkylene and heteroalkylene is optionally substituted with one or more R⁴; M and P are each independently C(R²) or N; X and Y are each independently C, C(R^{5a}) C(R^{5a})(R^{5b}), N, or N(R^{5c}), wherein the bond between X and Y may be a single or double bond as valency permits, and wherein X and Y may not both be C(R^{5a})(R^{5b}) or be C(R^{5a}); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R′; each R² is independently hydrogen, halo, cyano, C₁-C₆-alkyl, C₂-C₆-alkenyl, C₂-C₆-alkynyl, or —OR^{A}; each R⁴ is independently hydrogen, C₁-C₆-alkyl, C₁-C₆-heteroalkyl, C₁-C₆-haloalkyl, cycloalkyl, or heterocyclyl, wherein each alkyl, heteroalkyl, haloalkyl, cycloalkyl, and heterocyclyl is optionally substituted with one or more R¹²; each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁵, is hydrogen, C₁-C₆-alkyl, C₂-C₆-alkenyl, C₂-C₆-alkynyl, C₁-C₆-heteroalkyl, C₁-C₆-haloalkyl, halo, —NR^{B}R^{C}, or —ORF; R^{5b} is hydrogen or C₁-C₆-alkyl; or R^{5a} and R^{5b}, together with the carbon atom to which they are attached, form an oxo group; each R^{5c} is hydrogen, C₁-C₆-alkyl, C₁-C₆-haloalkyl, or C(O)R^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, —OR^{A}, —NR^{B}R^{C}, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹; or two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E} or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; RF is hydrogen, C₁-C₆ alkyl, cycloalkyl, heterocyclyl, aryl, or heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R¹² is independently deuterium, halo, cyano, —OR^{A}, —NR^{B}R^{C}, —N^{B}C(O)R^{D}, —C(O)NR^{B}R^{C}, —C(O)R^{D}, —C(O)OR^{D}, or —C(O)R^{D}; n is 0, 1, 2, 3, or 4; and x is 0, 1, or 2.

In another aspect, the present disclosure features a compound of Formula (I-a):

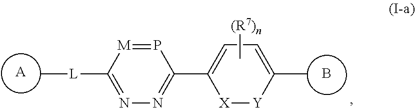

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —C(O)—, —N(R³)—, —S(O)ₓ—, —N(R³)C(O)—, or —C(O)N(R³)—, wherein each alkylene and heteroalkylene is optionally substituted with one or more R⁴; M and P are each independently C(R²) or N; X and Y are each independently C, C(R^{5a}) C(R^{5a})(R^{5b}), N, or N(R^{5c}), wherein the bond between X and Y may be a single or double bond as valency permits, and wherein X and Y may not both be C(R^{5a})(R^{5b}); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}—NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸; each R² is independently hydrogen, halo, cyano, C₁-C₆-alkyl, C₂-C₆-alkenyl, C₂-C₆-alkynyl, or —OR^{A}; each R³ is independently hydrogen, C₁-C₆-alkyl, or C₁-C₆-haloalkyl; each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R^{5a} is hydrogen, C₁-C₆-alkyl, C₂-C₆-alkenyl, C₂-C₆-alkynyl, C₁-C₆-heteroalkyl, C₁-C₆-haloalkyl, halo, —NR^{B}R^{C}, or —ORF; R^{5b} is hydrogen or C₁-C₆-alkyl; or R^{5a} and R^{5b}, together with the carbon atom to which they are attached, form an oxo group; each R^{5c} is hydrogen, C₁-C₆-alkyl, C₁-C₆-haloalkyl, or C(O)R^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, —OR^{A}, —NR^{B}R^{C}, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹; or two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E} or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; RF is hydrogen, C₁-C₆ alkyl, cycloalkyl, heterocyclyl, aryl, or heteroaryl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; n is 0, 1, 2, 3, or 4; and x is 0, 1, or 2.

As generally described herein, each of A or B are independently cycloalkyl, heterocyclyl, aryl, or heteroaryl, each of which is optionally substituted with one or more R¹.

In some embodiments, each of A and B are independently a monocyclic ring, e.g., monocyclic cycloalkyl, monocyclic heterocyclyl, monocyclic aryl, or monocyclic heteroaryl. The monocyclic ring may be saturated, partially unsaturated, or fully unsaturated (e.g., aromatic). In some embodiments, A or B are independently a monocyclic ring comprising between 3 and 10 ring atoms (e.g., 3, 4, 5, 6, 7, 8, 9, or 10 ring atoms). In some embodiments, A is a 4-membered monocyclic ring. In some embodiments, B is a 4-membered monocyclic ring. In some embodiments, A is a 5-membered monocyclic ring. In some embodiments, B is a 5-membered monocyclic ring. In some embodiments, A is a 6-membered monocyclic ring. In some embodiments, B is a 6-membered monocyclic ring. In some embodiments, A is a 7-membered monocyclic ring. In some embodiments, B is a 7-membered monocyclic ring. In some embodiments, A is an 8-membered monocyclic ring. In some embodiments, B is an 8-membered monocyclic ring. In some embodiments, A or B are independently a monocyclic ring optionally substituted with one or more R¹.

In some embodiments, A or B are independently a bicyclic ring, e.g., bicyclic cycloalkyl, bicyclic heterocyclyl, bicyclic aryl, or bicyclic heteroaryl. The bicyclic ring may be saturated, partially unsaturated, or fully unsaturated (e.g., aromatic). In some embodiments, A or B are independently a bicyclic ring comprising a fused, bridged, or spiro ring system. In some embodiments, A or B are independently a bicyclic ring comprising between 4 and 18 ring atoms (e.g., 4, 5, 6, 7, 8, 9, 10, 11, 12, 13, 14, 15, 16, 17, or 18 ring atoms). In some embodiments, A is a 6-membered bicyclic ring. In some embodiments, B is a 6-membered bicyclic ring. In some embodiments, A is a 7-membered bicyclic ring. In some embodiments, B is a 7-membered bicyclic ring. In some embodiments, A is an 8-membered bicyclic ring. In some embodiments, B is an 8-membered bicyclic ring. In some embodiments, A is a 9-membered bicyclic ring. In some embodiments, B is a 9-membered bicyclic ring. In some embodiments, A is a 10-membered bicyclic ring. In some embodiments, B is a 10-membered bicyclic ring. In some embodiments, A is an 11-membered bicyclic ring. In some embodiments, B is an 11-membered bicyclic ring. In some embodiments, A is a 12-membered bicyclic ring. In some embodiments, B is a 12-membered bicyclic ring. In some embodiments, A or B are independently a bicyclic ring optionally substituted with one or more R¹.

In some embodiments, A or B are independently a tricyclic ring, e.g., tricyclic cycloalkyl, tricyclic heterocyclyl, tricyclic aryl, or tricyclic heteroaryl. The tricyclic ring may be saturated, partially unsaturated, or fully unsaturated (e.g., aromatic). In some embodiments, A or B are independently a tricyclic ring that comprises a fused, bridged, or spiro ring system, or a combination thereof. In some embodiments, A or B are independently a tricyclic ring comprising between 6 and 24 ring atoms (e.g., 6, 7, 8, 9, 10, 11, 12, 13, 14, 15, 16, 17, 18, 19, 20, 21, 22, 23, or 24 ring atoms). In some embodiments, A is an 8-membered tricyclic ring. In some embodiments, B is an 8-membered tricyclic ring. In some embodiments, A is a 9-membered tricyclic ring. In some embodiments, B is a 9-membered tricyclic ring. In some embodiments, A is a 10-membered tricyclic ring. In some embodiments, B is a 10-membered tricyclic ring. In some embodiments, A or B are independently a tricyclic ring optionally substituted with one or more R¹.

In some embodiments, A or B are independently monocyclic cycloalkyl, monocyclic heterocyclyl, monocyclic aryl, or monocyclic heteroaryl. In some embodiments, A or B are independently bicyclic cycloalkyl, bicyclic heterocyclyl, bicyclic aryl, or bicyclic heteroaryl. In some embodiments, A or B are independently tricyclic cycloalkyl, tricyclic heterocyclyl, tricyclic aryl, or tricyclic heteroaryl. In some embodiments, A is monocyclic heterocyclyl. In some embodiments, B is monocyclic heterocyclyl. In some embodiments, A is bicyclic heterocyclyl. In some embodiments, B is bicyclic heterocyclyl. In some embodiments, A is monocyclic heteroaryl. In some embodiments, B is monocyclic heteroaryl. In some embodiments, A is bicyclic heteroaryl. In some embodiments, B is bicyclic heteroaryl.

In some embodiments, A or B are independently a nitrogen-containing heterocyclyl, e.g., heterocyclyl comprising one or more nitrogen atom. The one or more nitrogen atom of the nitrogen-containing heterocyclyl may be at any position of the ring. In some embodiments, the nitrogen-containing heterocyclyl is monocyclic, bicyclic, or tricyclic ring. In some embodiments, A or B are independently heterocyclyl comprising at least 1, at least 2, at least 3, at least 4, at least 5, or at least 6 nitrogen atoms. In some embodiments, A is heterocyclyl comprising 1 nitrogen atom. In some embodiments, B is heterocyclyl comprising 1 nitrogen atom. In some embodiments, A is heterocyclyl comprising 2 nitrogen atoms. In some embodiments, B is heterocyclyl comprising 2 nitrogen atoms. In some embodiments, A is heterocyclyl comprising 3 nitrogen atoms. In some embodiments, B is heterocyclyl comprising 3 nitrogen atoms. In some embodiments, A is heterocyclyl comprising 4 nitrogen atoms. In some embodiments, B is heterocyclyl comprising 4 nitrogen atoms. In some embodiments, A or B are independently a nitrogen-containing heterocyclyl comprising one or more additional heteroatoms, e.g., one or more of oxygen, sulfur, boron, silicon, or phosphorus. In some embodiments, the one or more nitrogen of the nitrogen-containing heterocyclyl is substituted, e.g., with R¹.

In some embodiments, A or B are independently a nitrogen-containing heteroaryl, e.g., heteroaryl comprising one or more nitrogen atom. The one or more nitrogen atom of the nitrogen-containing heteroaryl may be at any position of the ring. In some embodiments, the nitrogen-containing heteroaryl is monocyclic, bicyclic, or tricyclic. In some embodiments, A or B are independently heteroaryl comprising at least 1, at least 2, at least 3, at least 4, at least 5, or at least 6 nitrogen atoms. In some embodiments, A is heteroaryl comprising 1 nitrogen atom. In some embodiments, B is heteroaryl comprising 1 nitrogen atom. In some embodiments, A is heteroaryl comprising 2 nitrogen atoms. In some embodiments, B is heteroaryl comprising 2 nitrogen atoms. In some embodiments, A is heteroaryl comprising 3 nitrogen atoms. In some embodiments, B is heteroaryl comprising 3 nitrogen atoms. In some embodiments, A is heteroaryl comprising 4 nitrogen atoms. In some embodiments, B is heteroaryl comprising 4 nitrogen atoms. In some embodiments, A or B are independently a nitrogen-containing heteroaryl comprising one or more additional heteroatoms, e.g., one or more of oxygen, sulfur, boron, silicon, or phosphorus. In some embodiments, the one or more nitrogen of the nitrogen-containing heteroaryl is substituted, e.g., with R¹.

In some embodiments, A is a 6-membered nitrogen-containing heterocyclyl, e.g., a 6-membered heterocyclyl comprising one or more nitrogen. In some embodiments, A is a 6-membered heterocyclyl comprising 1 nitrogen atom. In some embodiments, A is a 6-membered heterocyclyl comprising 2 nitrogen atoms. In some embodiments, A is a 6-membered heterocyclyl comprising 3 nitrogen atoms. In some embodiments, A is a 6-membered heterocyclyl comprising 4 nitrogen atoms. The one or more nitrogen atom of the 6-membered nitrogen-containing heterocyclyl may be at any position of the ring. In some embodiments, A is a 6-membered nitrogen-containing heterocyclyl optionally substituted with one or more R¹. In some embodiments, the one or more nitrogen of the 6-membered nitrogen-containing heterocyclyl is substituted, e.g., with R¹. In some embodiments, A is a 6-membered nitrogen-containing heterocyclyl comprising one or more additional heteroatoms, e.g., one or more of oxygen, sulfur, boron, silicon, or phosphorus.

In some embodiments, B is a 5-membered nitrogen-containing heterocyclyl or heteroaryl, e.g., a 5-membered heterocyclyl or heteroaryl comprising one or more nitrogen. In some embodiments, B is a 5-membered heterocyclyl comprising 1 nitrogen atom. In some embodiments, B is a 5-membered heteroaryl comprising 1 nitrogen atom. In some embodiments, B is a 5-membered heterocyclyl comprising 2 nitrogen atoms. In some embodiments, B is a 5-membered heteroaryl comprising 2 nitrogen atoms. In some embodiments, B is a 5-membered heterocyclyl comprising 3 nitrogen atoms. In some embodiments, B is a 5-membered heteroaryl comprising 3 nitrogen atoms. The one or more nitrogen atom of the 5-membered nitrogen-containing heterocyclyl or heteroaryl may be at any position of the ring. In some embodiments, B is a 5-membered nitrogen-containing heterocyclyl optionally substituted with one or more R¹. In some embodiments, B is a 5-membered nitrogen-containing heteroaryl optionally substituted with one or more R¹. In some embodiments, the one or more nitrogen of the 5-membered nitrogen-containing heterocyclyl or heteroaryl is substituted, e.g., with R¹. In some embodiments, B is a 5-membered nitrogen-containing heterocyclyl or heteroaryl comprising one or more additional heteroatoms, e.g., one or more of oxygen, sulfur, boron, silicon, or phosphorus.

In some embodiments, each of A and B are independently selected from:

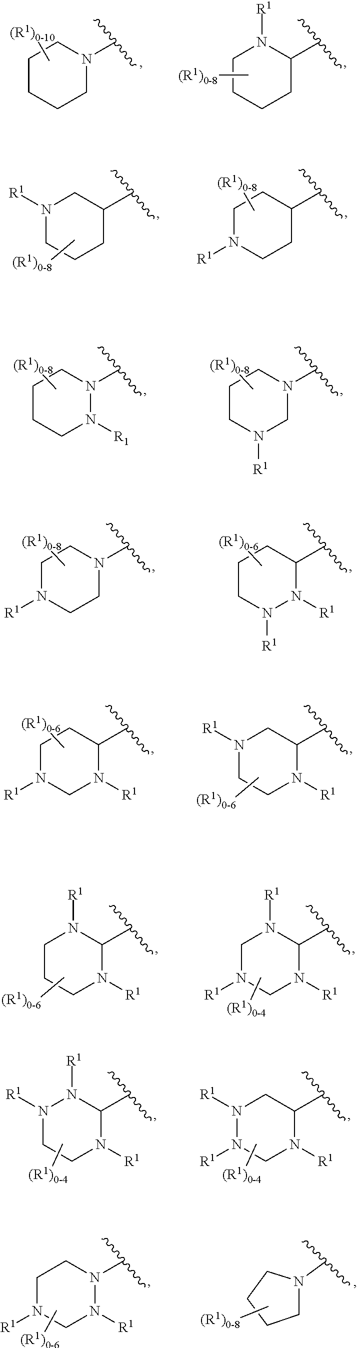

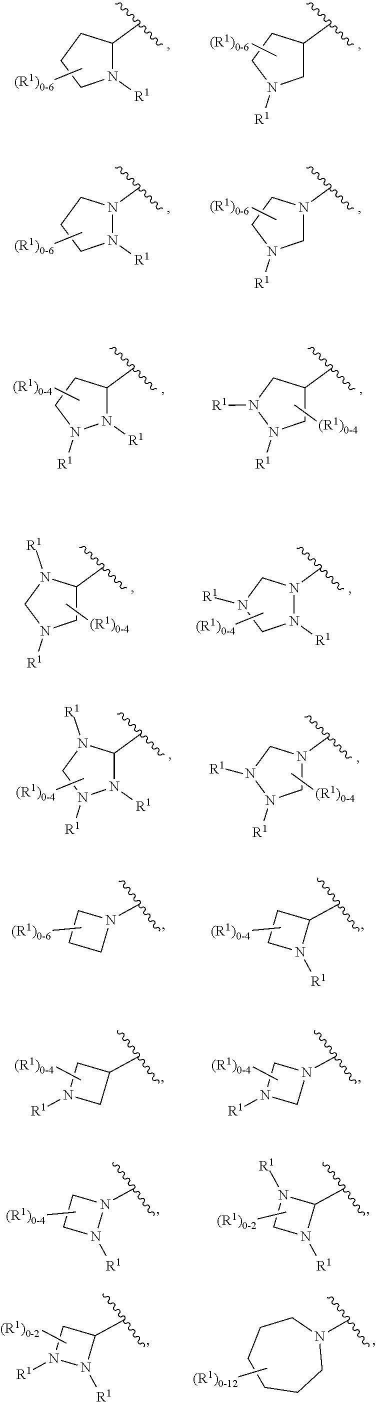

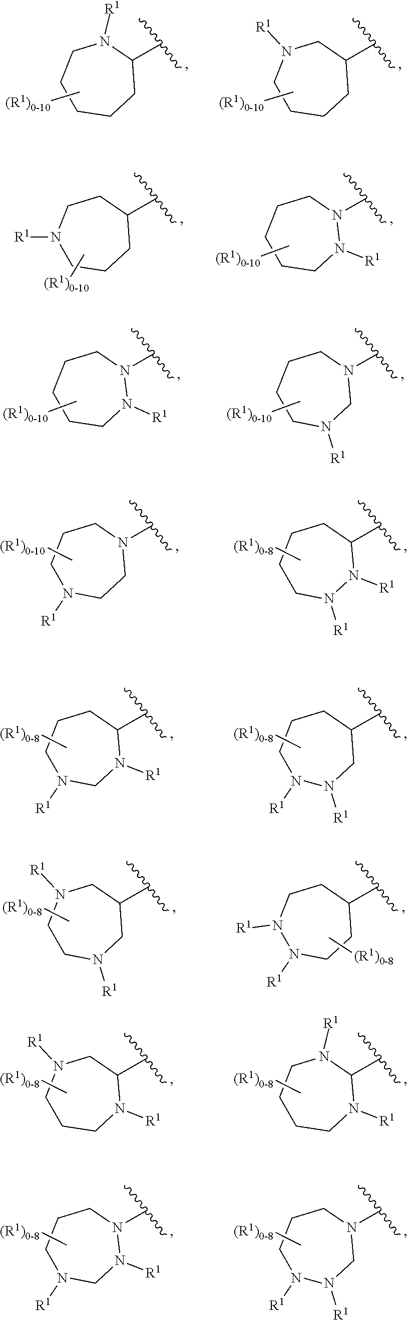

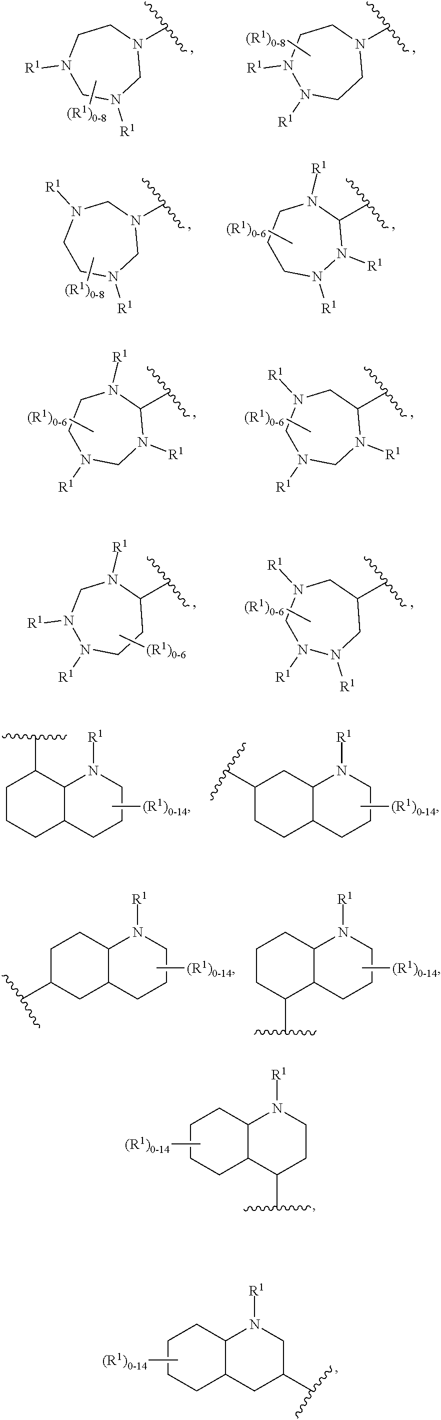

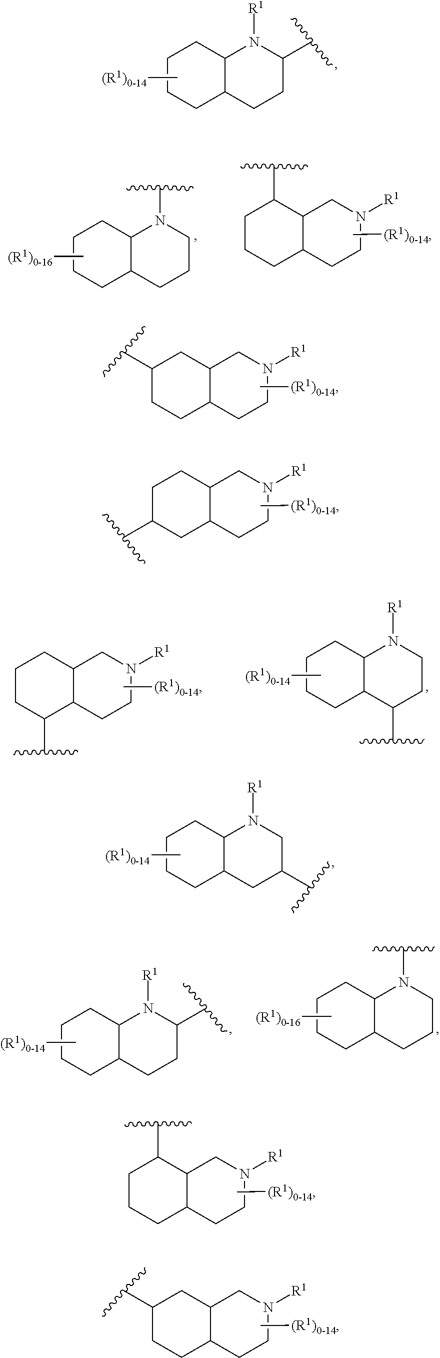

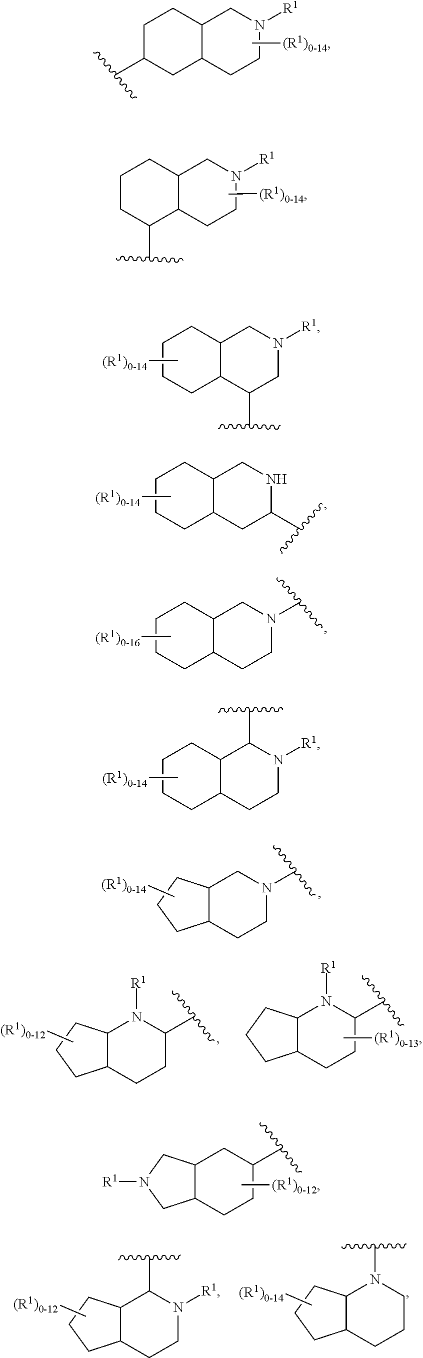

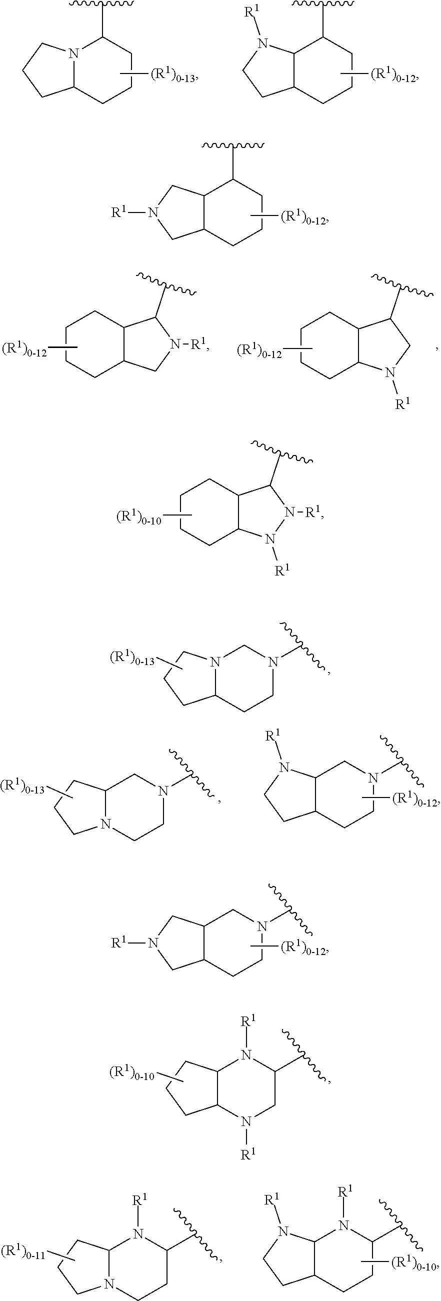

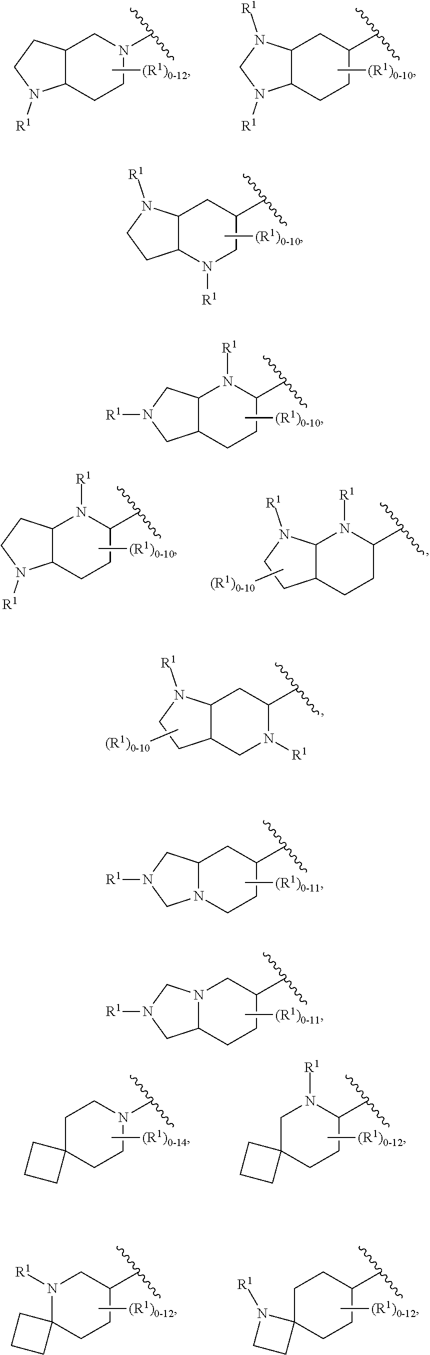

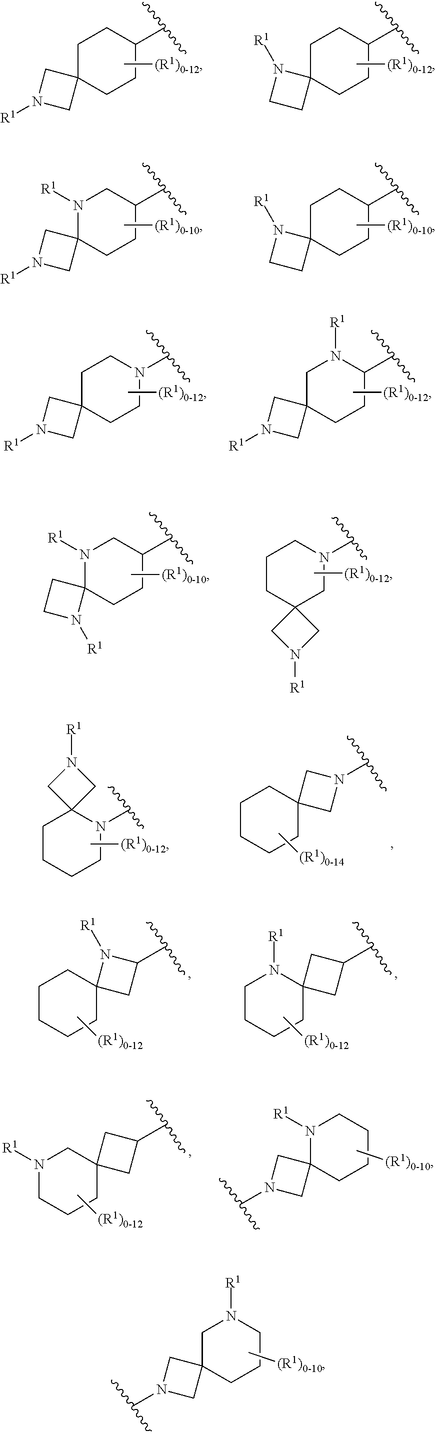

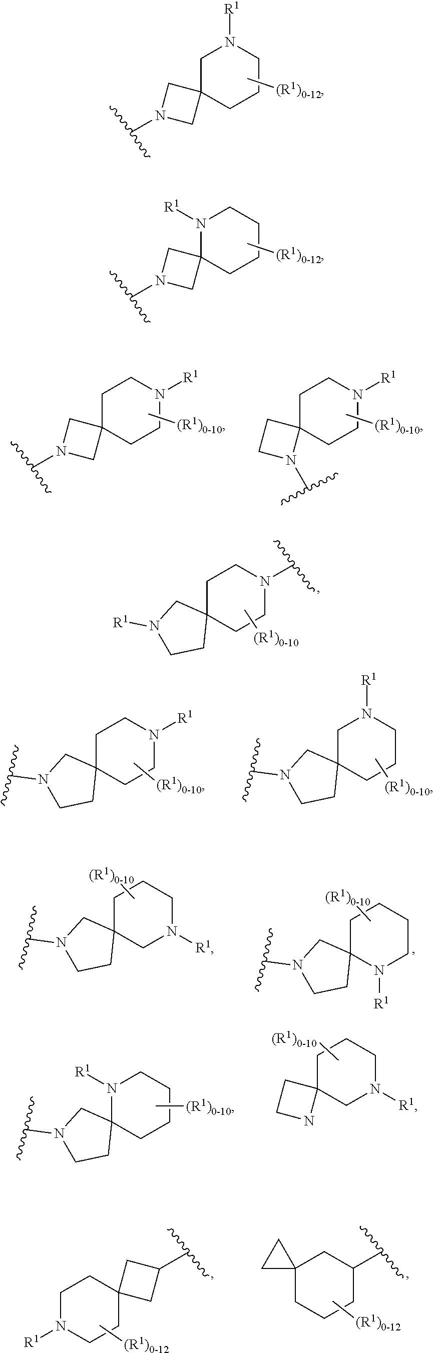

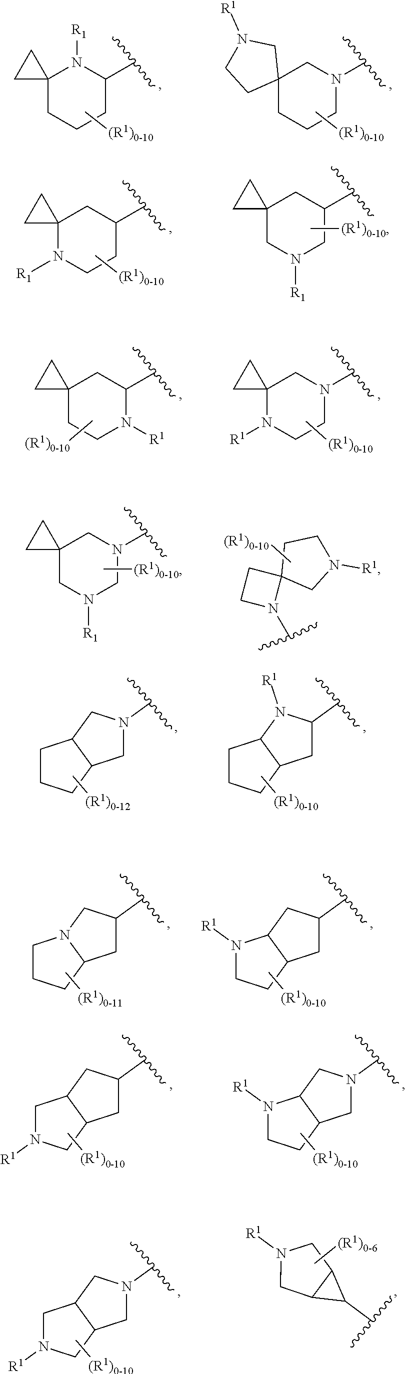

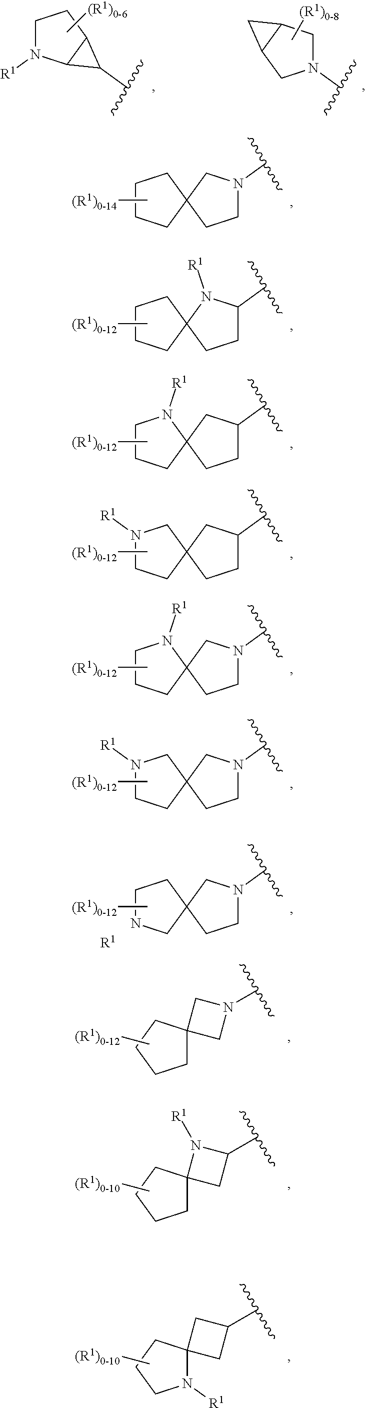

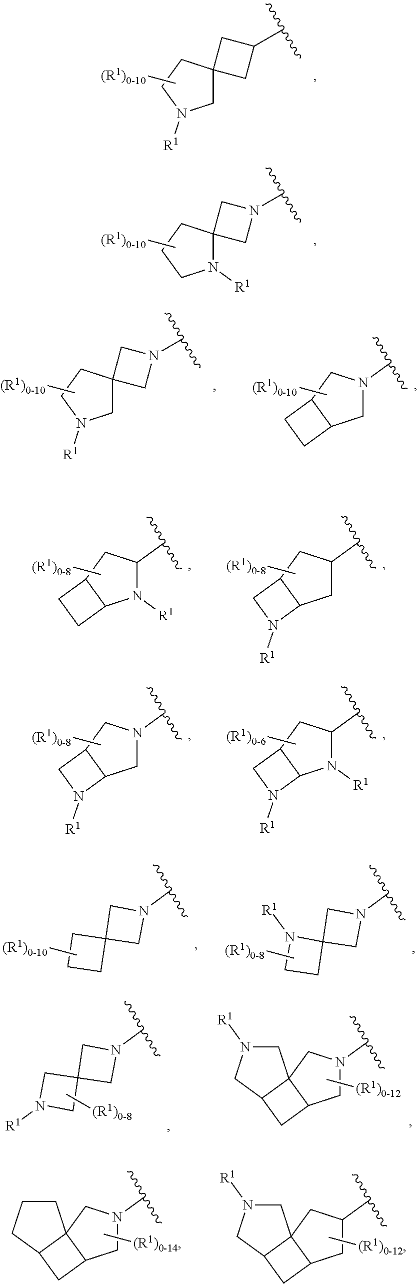

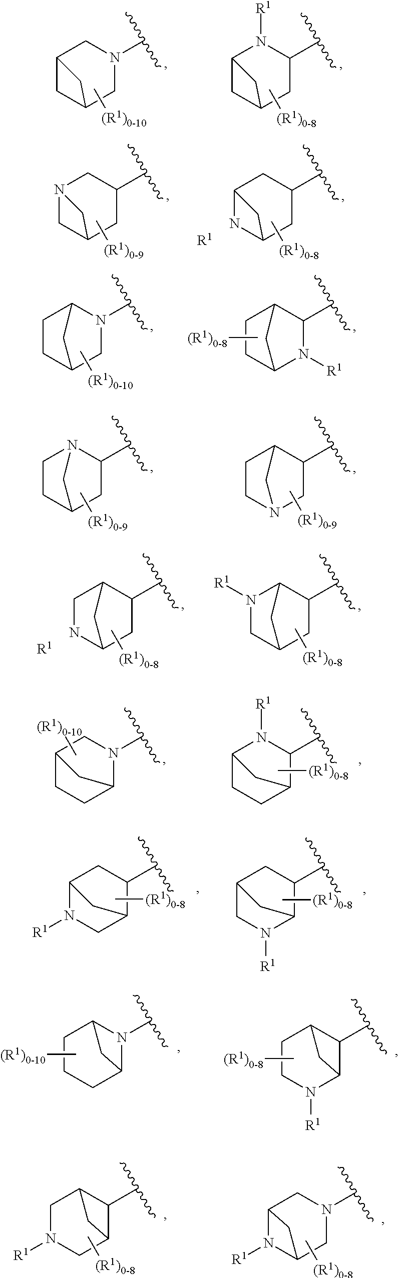

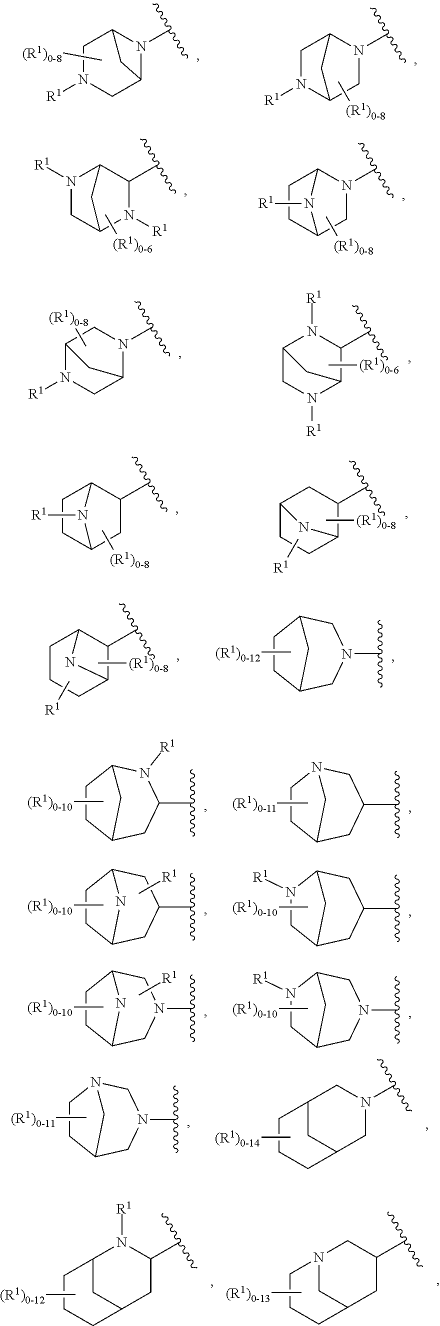

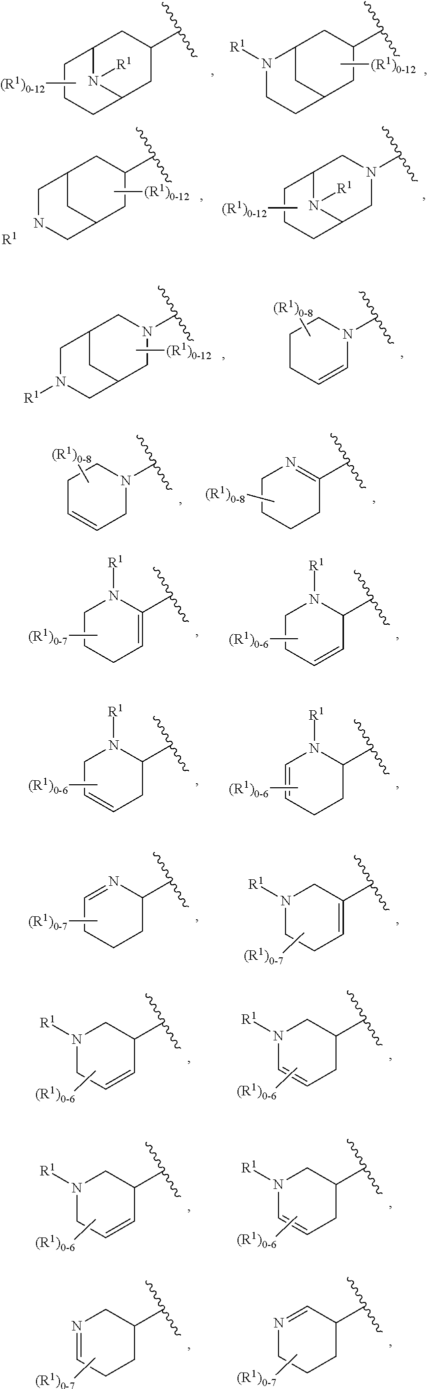

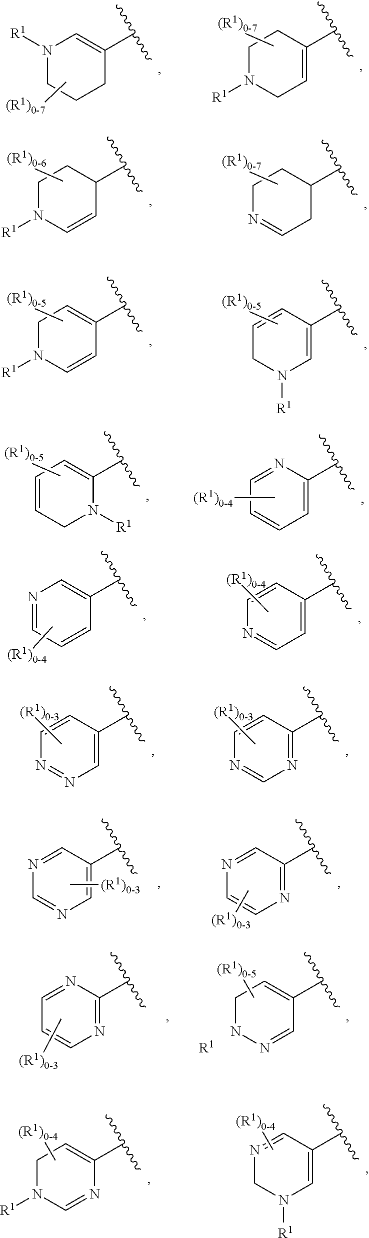

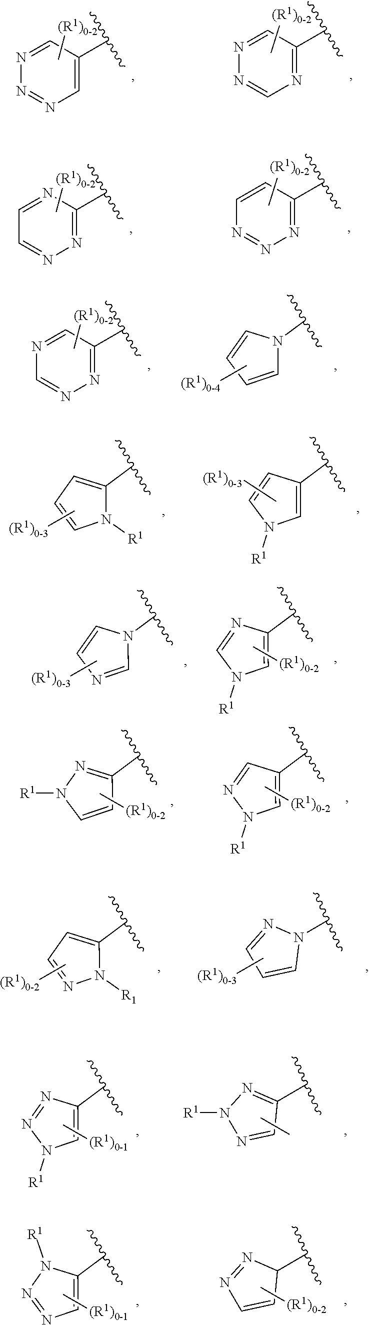

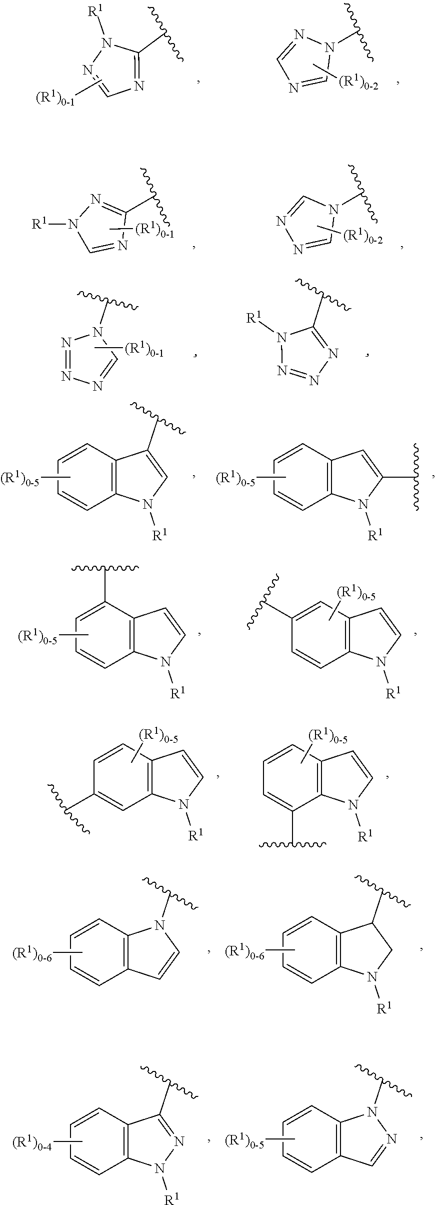

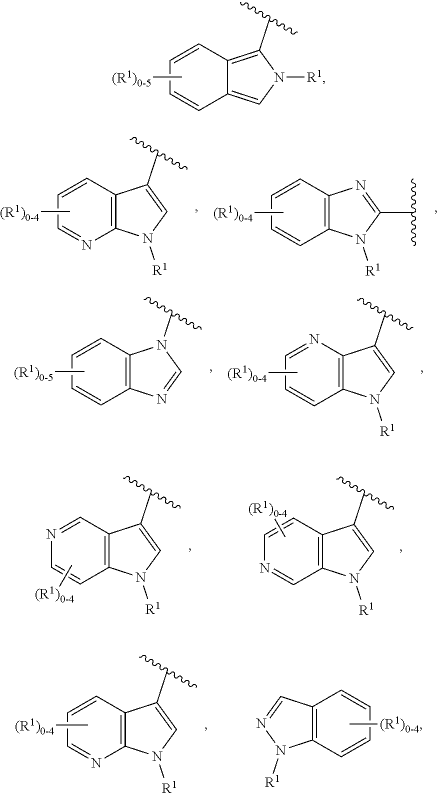

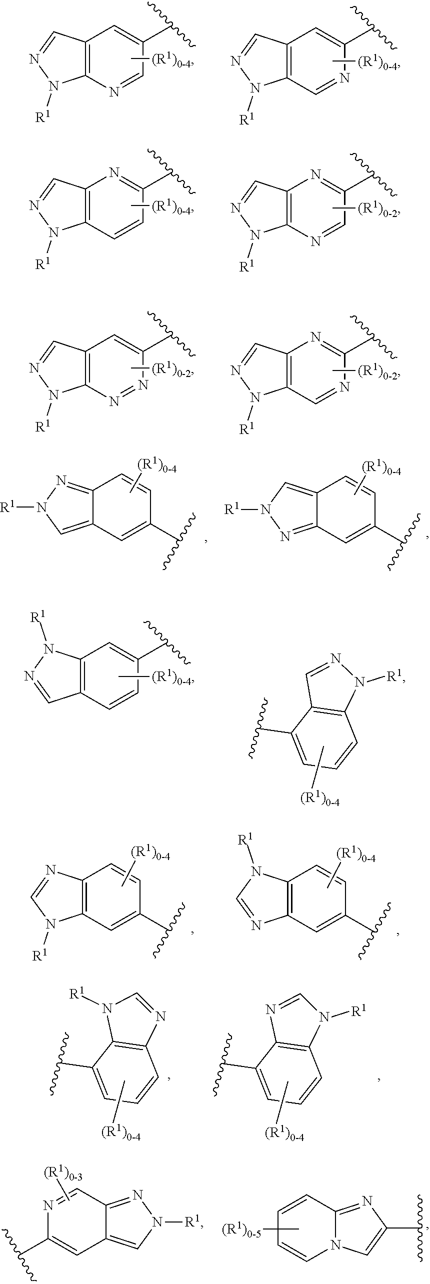

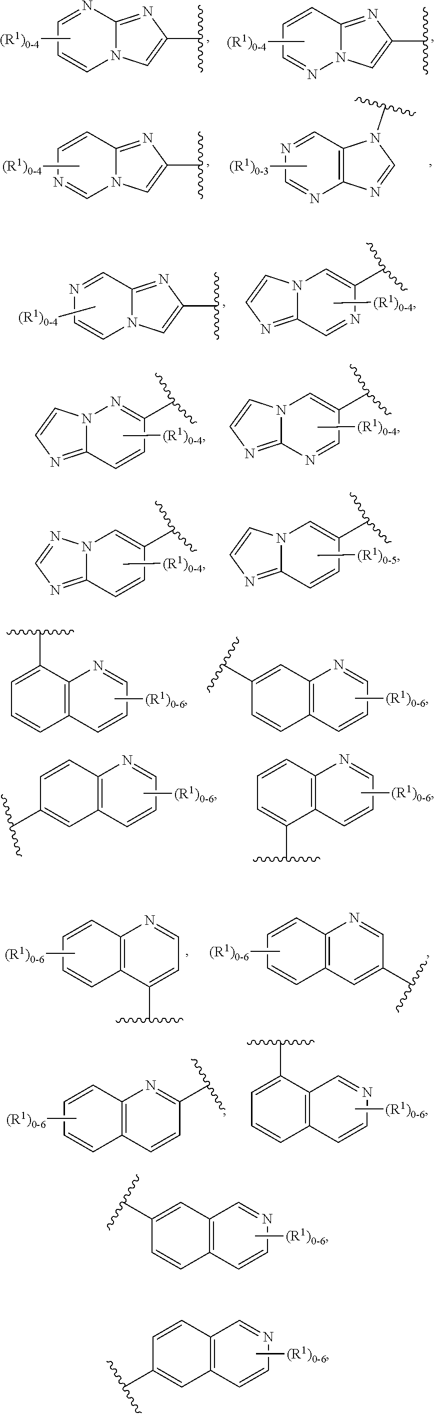

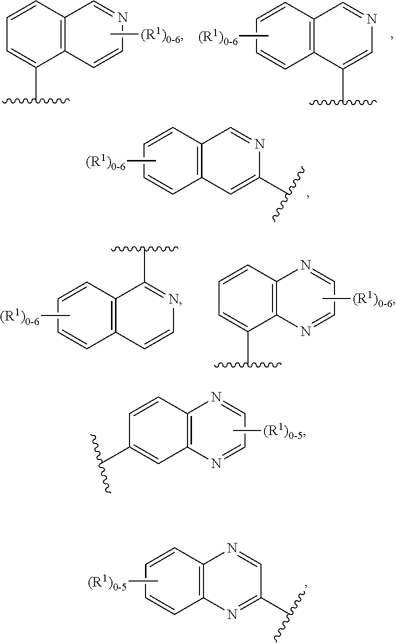

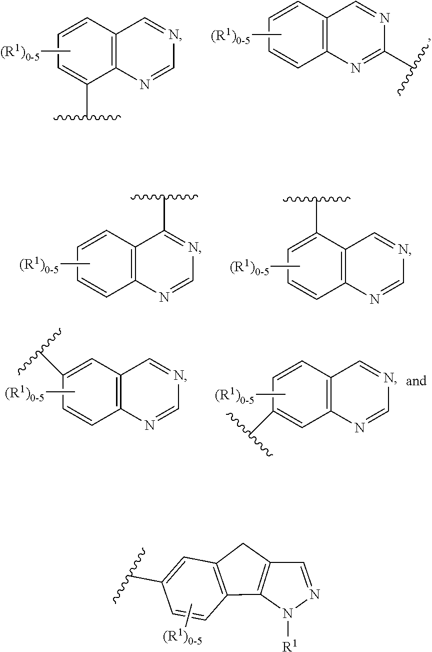

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, each of A and B are independently selected from:

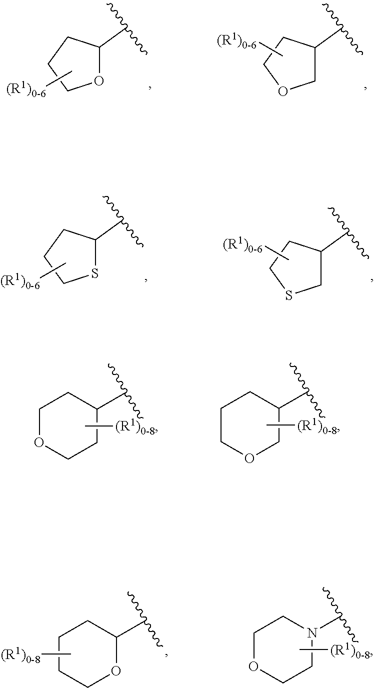

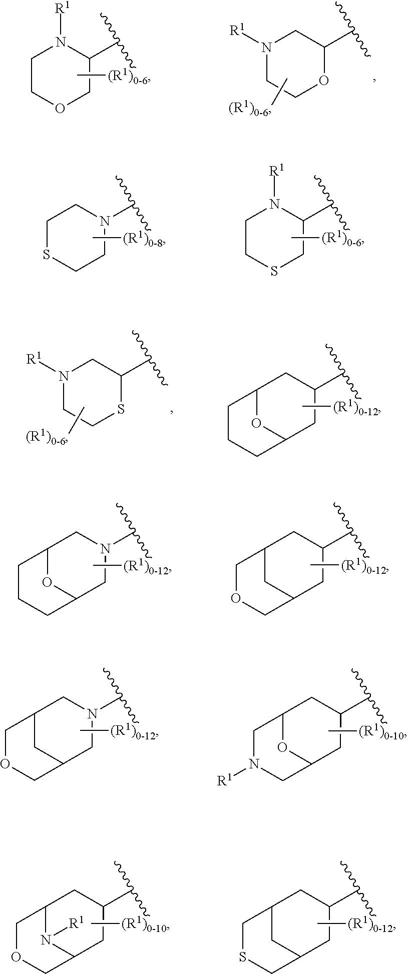

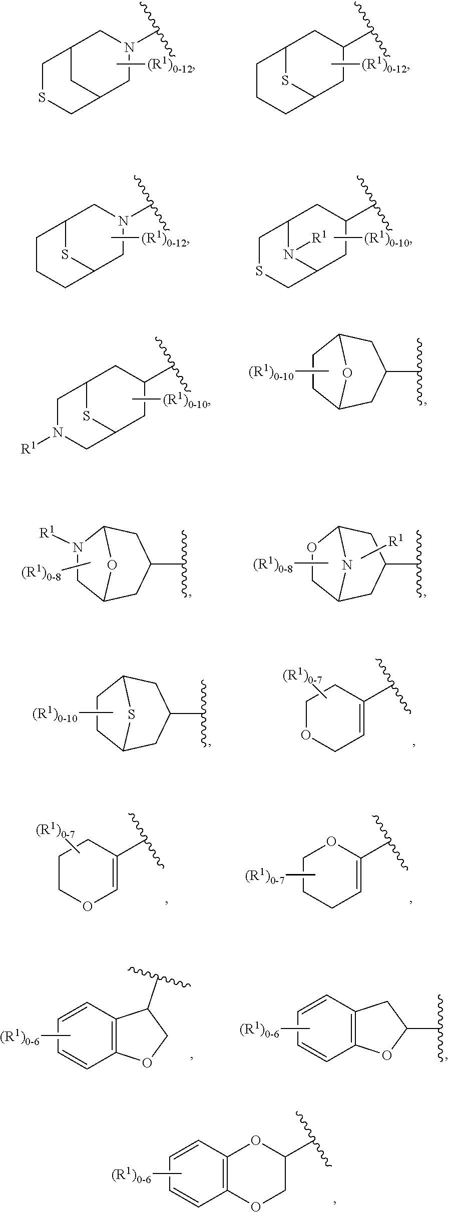

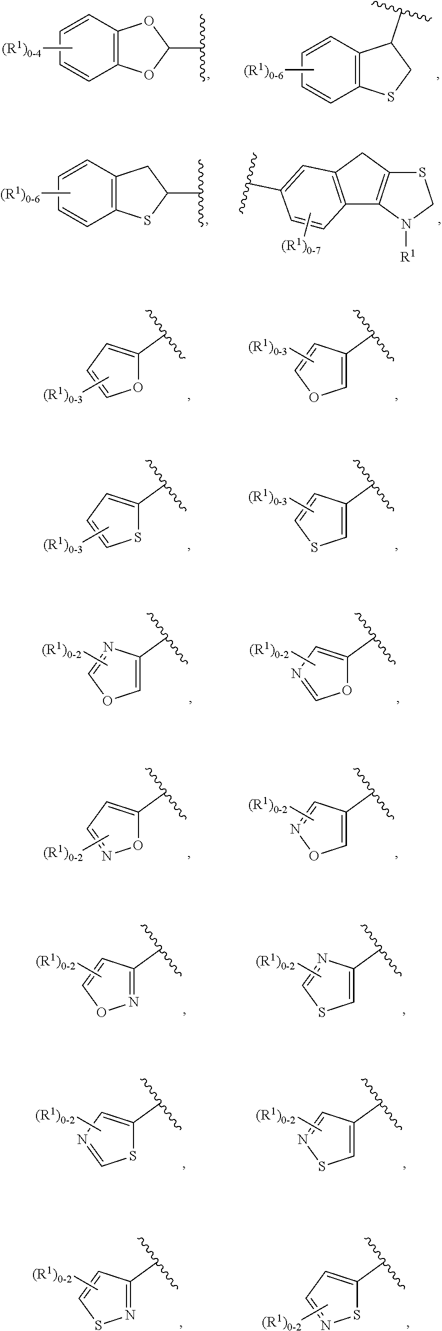

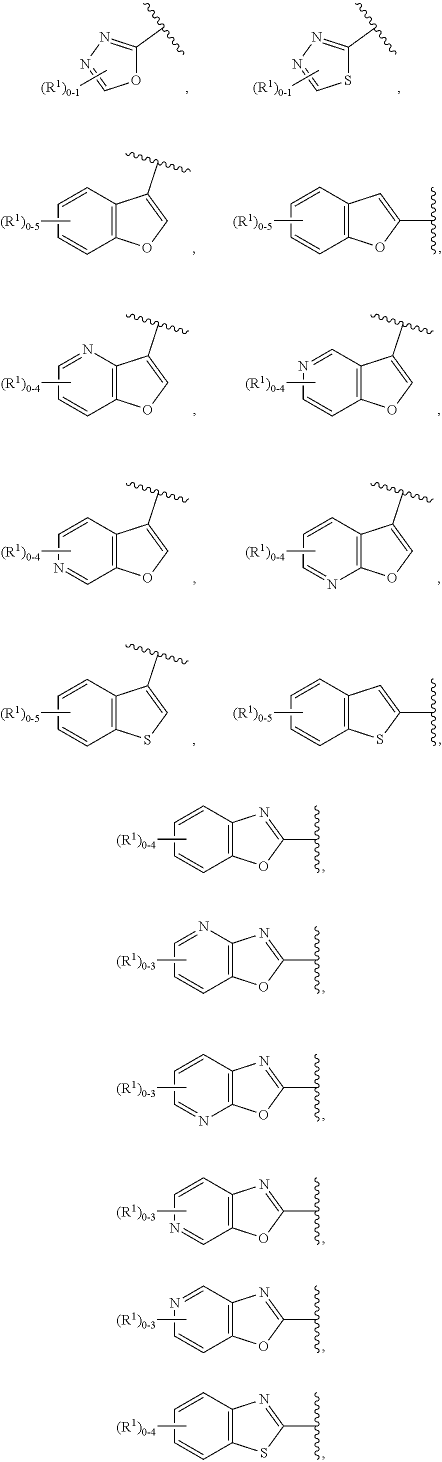

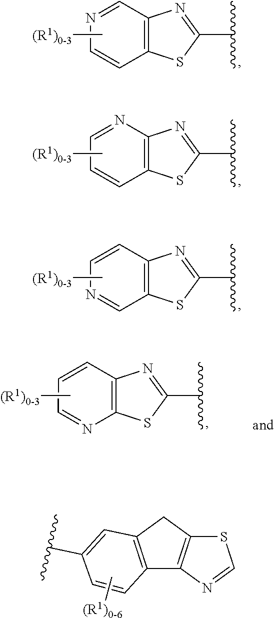

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, A is selected from

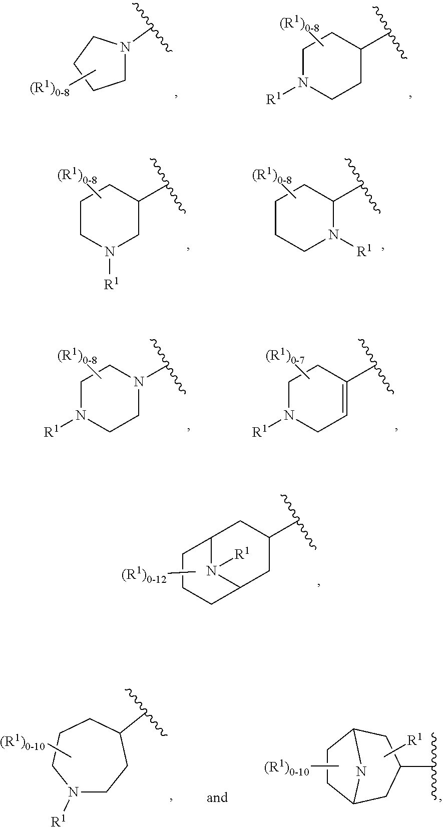

wherein R¹ is as defined herein.

In some embodiments, A is selected from

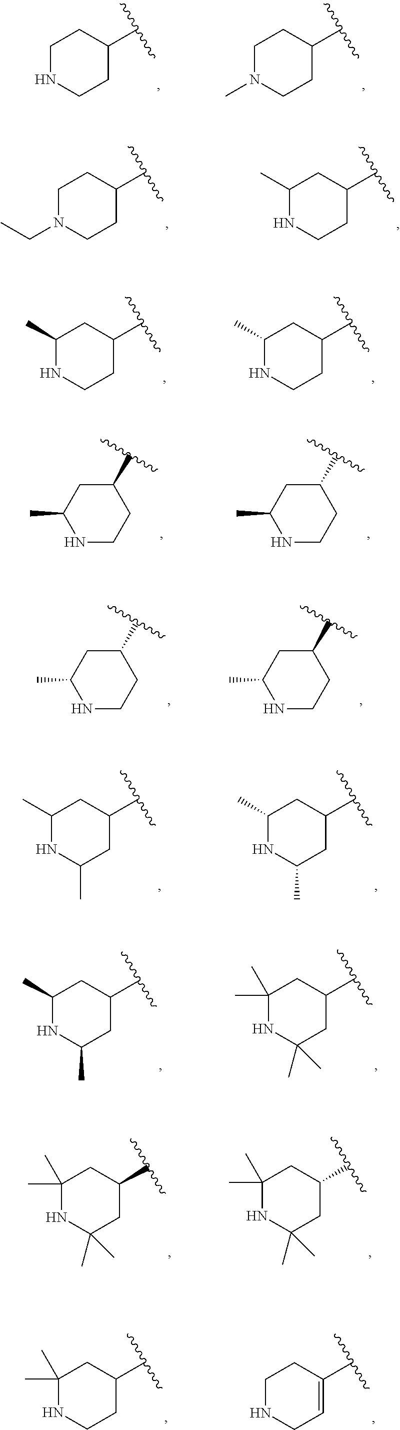

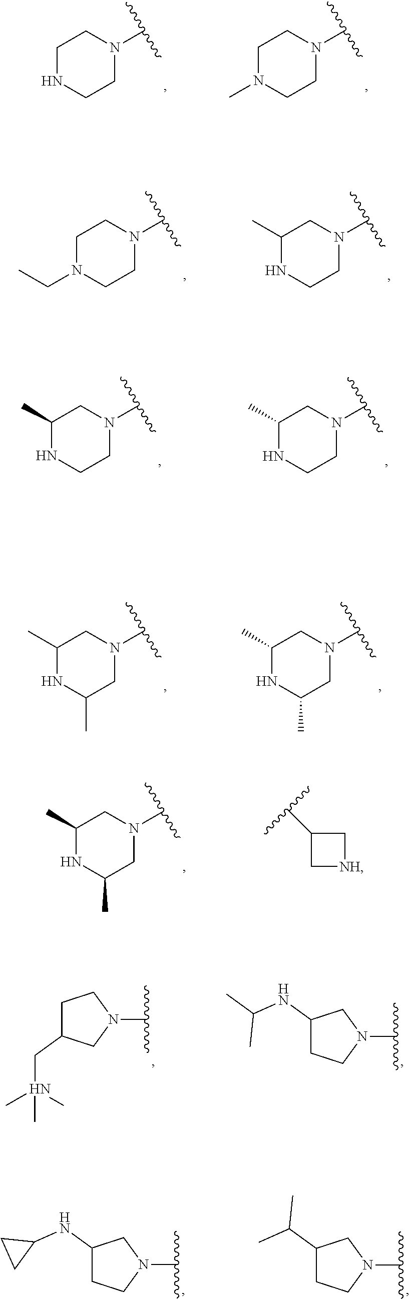

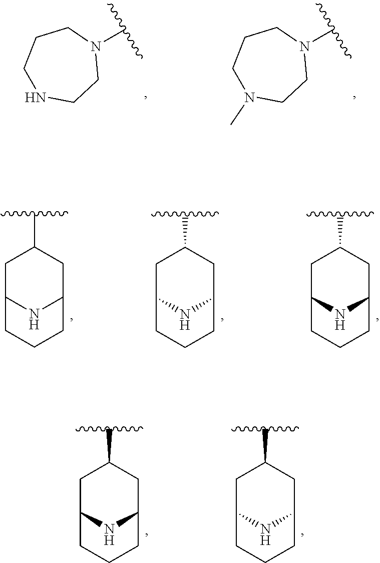

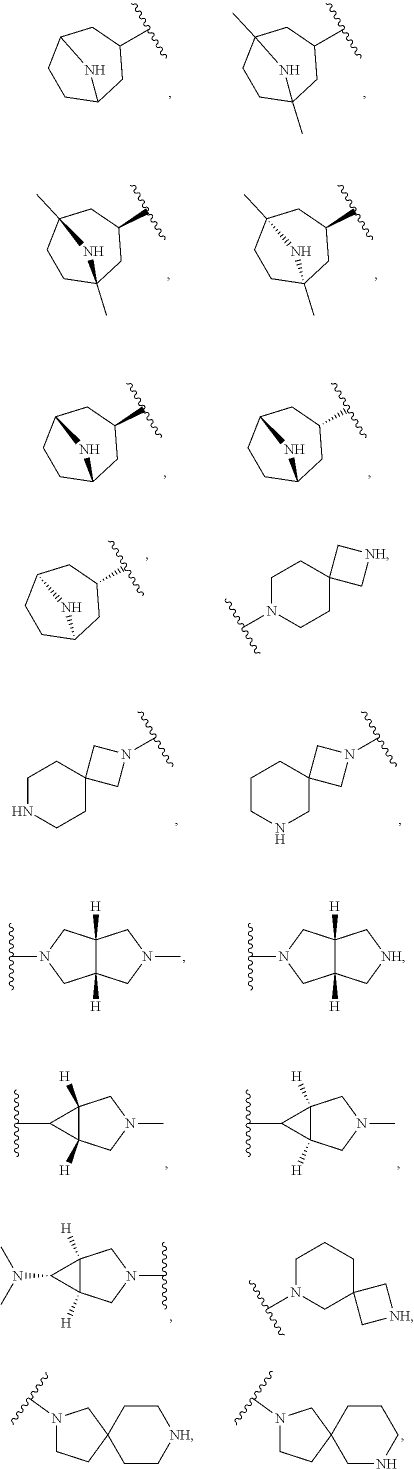

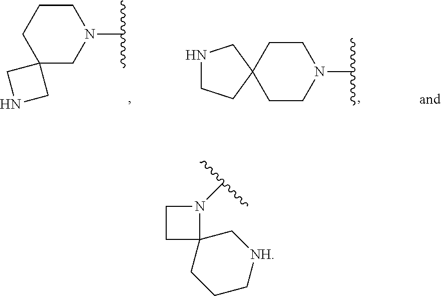 [SMILES: *N1CCCC2(CNC2)C1 ; *N1CCC2(CCNC2)CC1 ; *N1CCC12CCCNC2]

In some embodiments, A is selected from

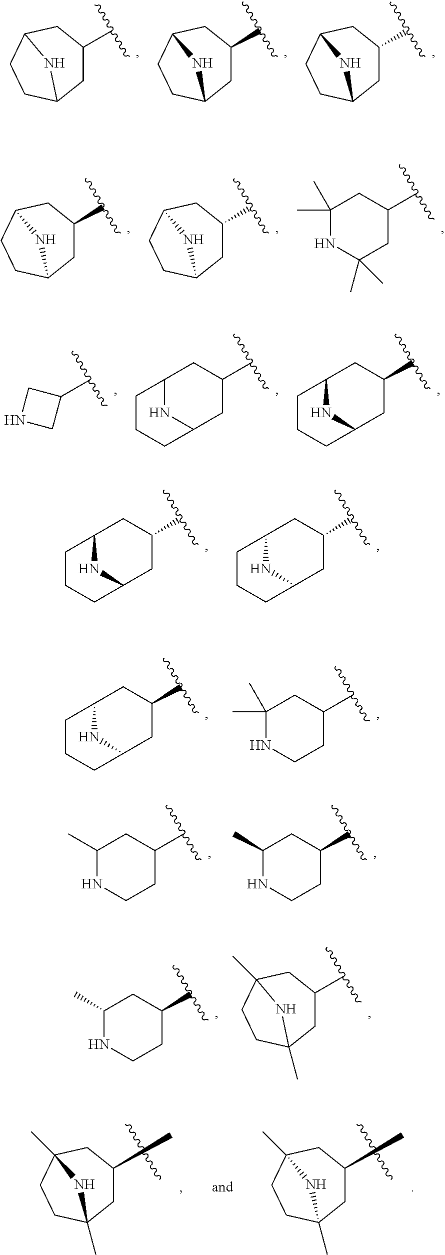

In some embodiments, A is selected from

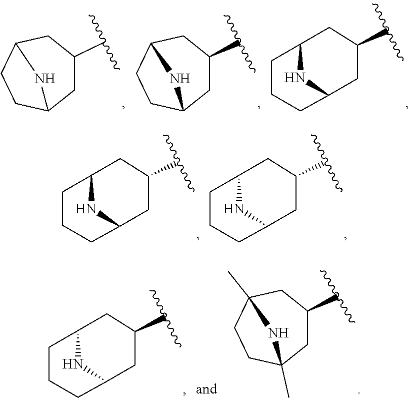

In some embodiments, A is

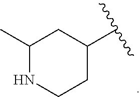 [SMILES: *C1CCNC(C)C1]

In some embodiments, A is

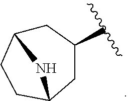 [SMILES: *[C@@H]1C[C@H]2CC[C@@H](C1)N2]

In some embodiments, A is

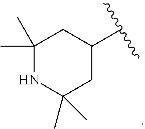 [SMILES: *C1CC(C)(C)NC(C)(C)C1]

In some embodiments, A is

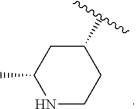 [SMILES: *[C@@H]1CCN[C@H](C)C1]

In some embodiments, A is

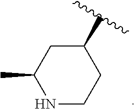 [SMILES: *[C@H]1CCN[C@@H](C)C1]

In some embodiments, A is

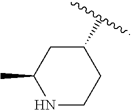

In some embodiments, A is

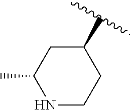

In some embodiments, A is

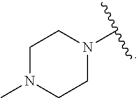 [SMILES: *N1CCN(C)CC1]

In some embodiments, A is

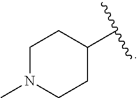 [SMILES: *C1CCN(C)CC1]

In some embodiments, A is

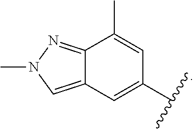 [SMILES: *c1cc(C)c2nn(C)cc2c1]

In some embodiments, A is

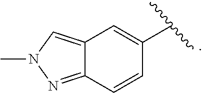 [SMILES: *c1ccc2nn(C)cc2c1]

In some embodiments, B is selected from

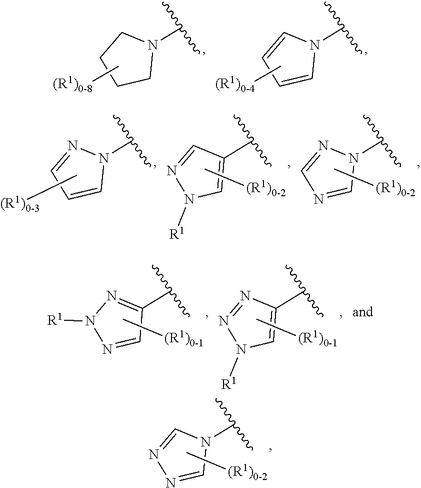

wherein R¹ is as defined herein. In some embodiments, B is selected from

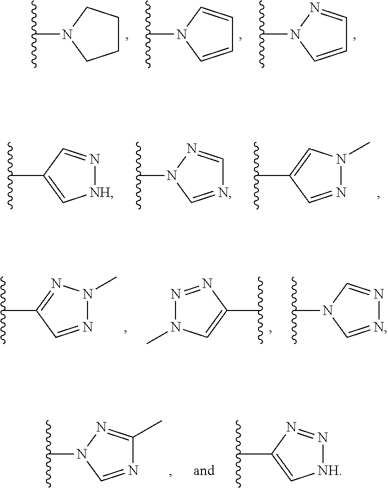

In some embodiments, B is

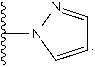 [SMILES: *n1cccn1]

In some embodiments, B is

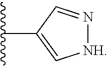 [SMILES: *c1cn[nH]c1]

In some embodiments, B is

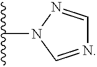 [SMILES: *n1cncn1]

In some embodiments, B is

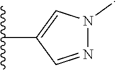

In some embodiments, B is

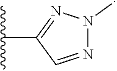

In some embodiments, B is

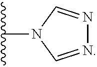 [SMILES: *n1cnnc1]

In some embodiments, B is

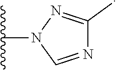

In some embodiments, B is

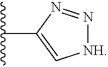 [SMILES: *c1c[nH]nn1]

In some embodiments, B is

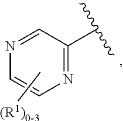

wherein R¹ is as defined herein.

In some embodiments, B is selected from

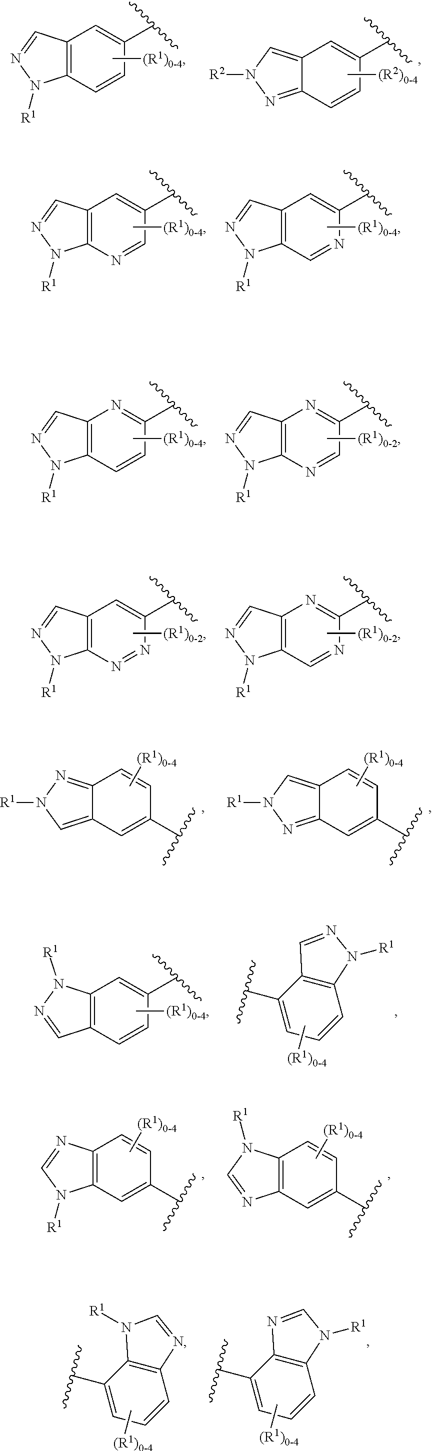

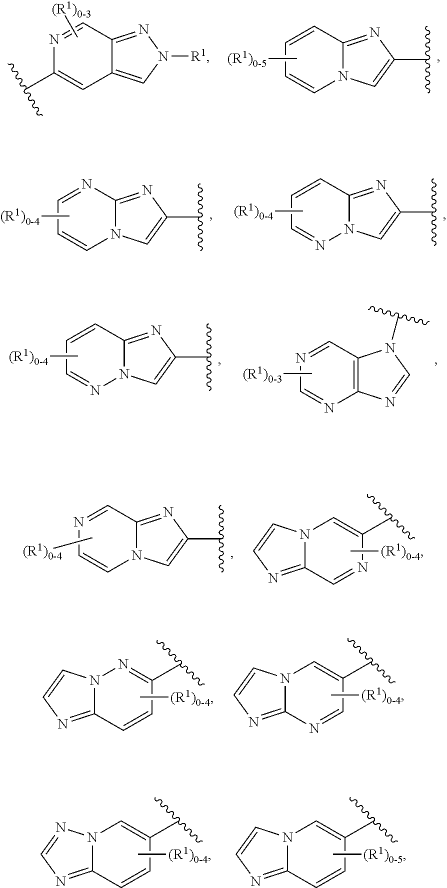

wherein R¹ is as defined herein. In some embodiment, B is selected from

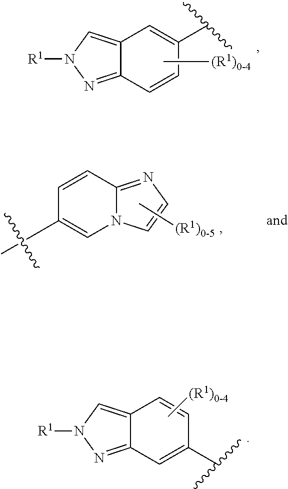

In some embodiments, B is

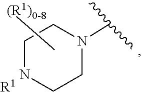

wherein R¹ is as defined herein. In some embodiment, B is selected from

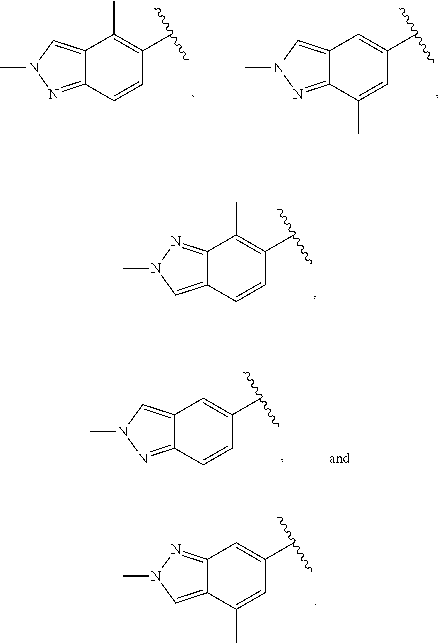 [SMILES: *c1ccc2nn(C)cc2c1C ; *c1cc(C)c2nn(C)cc2c1 ; *c1ccc2cn(C)nc2c1C ; *c1ccc2nn(C)cc2c1 ; *c1cc(C)c2cn(C)nc2c1]

In some embodiments, B is

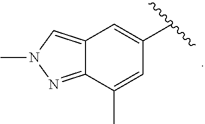 [SMILES: *c1cc(C)c2nn(C)cc2c1]

In some embodiments, B is

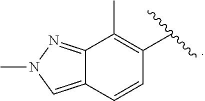 [SMILES: *c1ccc2cn(C)nc2c1C]

In some embodiments, B is

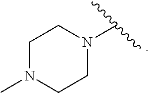 [SMILES: *N1CCN(C)CC1]

In some embodiments, B is

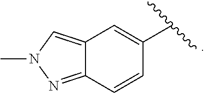 [SMILES: *c1ccc2nn(C)cc2c1]

In some embodiments, B is

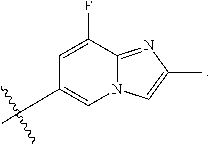 [SMILES: *c1cc(F)c2nc(C)cn2c1]

In some embodiments, B is

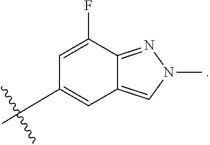 [SMILES: *c1cc(F)c2nn(C)cc2c1]

In some embodiments, B is

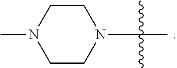 [SMILES: *N1CCN(C)CC1]

In some embodiments, B is

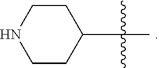 [SMILES: *C1CCNCC1]

As generally described herein, L may be absent or refer to a C₁-C₆-alkylene, C₁-C₆-heteroalkylene, —O—, —C(O)—, —N(R³)—, —S(O)ₓ, —N(R³)C(O)—, or —C(O)N(R³)— group, wherein each alkylene and heteroalkylene is optionally substituted with one or more R⁴. In some embodiments, L may be absent or refer to a C₁-C₆-alkylene, C₁-C₆-heteroalkylene, —O—, —C(O)—, —N(R³)—, —N(R³)C(O)—, or —C(O)N(R³)— group, wherein each alkylene and heteroalkylene is optionally substituted with one or more R⁴.

In some embodiments, L is absent. In some embodiments, L is C₁-C₆-alkylene (e.g., C₁-alkylene, C₂-alkylene, C₃-alkylene, C₄-alkylene, C₅-alkylene, or C₆-alkylene). In some embodiments, L is unsubstituted C₁-C₆ alkylene. In some embodiments, L is substituted C₁-C₆-alkylene, e.g., C₁-C₆ alkylene substituted with one or more R⁴. In some embodiments, L is C₁-alkylene substituted with one R⁴. In some embodiments, L is —CH₂— (or methylene). In some embodiments, L is —C(O)— (or carbonyl).

In some embodiments, L is absent, C₁-C₆-alkylene, C₁-C₆-heteroalkylene, —N(R³)C(O)—, or —C(O)N(R³)—, wherein each alkylene and heteroalkylene is optionally substituted with one or more R⁴.

In some embodiments, L is C₁-C₆-heteroalkylene (e.g., C₁-heteroalkylene, C₂-heteroalkylene, C₃-heteroalkylene, C₄-heteroalkylene, C₅-heteroalkylene, or C₆-heteroalkylene). In some embodiments, L is unsubstituted C₁-C₆ heteroalkylene. In some embodiments, L is C₁-C₆-heteroalkylene substituted with one or more R⁴. In some embodiments, the heteroalkylene comprises 1 or more heteroatoms. In some embodiments, the heteroalkylene comprises one or more of oxygen, sulfur, nitrogen, boron, silicon, or phosphorus. In some embodiments, L is —N(R³)C(O)—. In some embodiments, L is —C(O)N(R³)—.

In some embodiments, L is oxygen. In some embodiments, L is nitrogen, which may be substituted with R³. In some embodiments, L is nitrogen substituted with one R³. In some embodiments, L is —N(R³)—. In some embodiments, L is —N(CH₃)—. In some embodiments, L is —NH—. In some embodiments, L is —O—.

In some embodiments, L is —S(O)ₓ—. In some embodiments, x is 0, 1, or 2. In some embodiments, L is —S— or —S(O)₂—. In some embodiments, L is —S—.

As generally described herein, each of M and P independently refer to C(R²) or N. In some embodiments, each of M and P is independently C(R²) or N. In some embodiments, M and P are each independently C(R²), e.g., CH. In some embodiments, one of M and P is C(R²), and the other of M and P is N. In some embodiments, M is C(R²). In some embodiments, M is N. In some embodiments, P is C(R²). In some embodiments, P is N. In some embodiments, M is C(R²) (e.g., CH) and P is N. In some embodiments, M is N and P is C(R²) (e.g., CH).

In some embodiments,

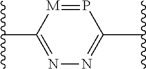

is selected from

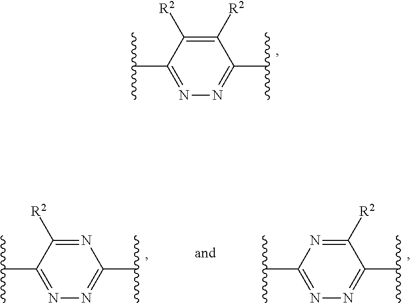

wherein R² is as defined above. In some embodiments, R² is hydrogen.

As generally described herein, each of X and Y independently refer to C, C(R^{5a}) C(R^{5a})(R^{5b}), N, or N(R^{5c}). In some embodiments, each of X and Y are independently C. In some embodiments, each of X and Y are C(R^{5a}). In some embodiments, X and Y are may not both be C(R^{5a}).

In some embodiments, when Y is N and X is C(R^{5a}) (e.g., CH), L is not —N(R³)— (e.g., —N(CH₃)—). In some embodiments, when Y is N and X is CH, L is not —N(R³)— (e.g., —N(CH₃)—). In some embodiments, when Y is N and X is C(R^{5a}) (e.g., CH), L is not —N(CH₃)—. In some embodiments, when Y is N and X is CH, L is not —N(CH₃)—. In some embodiments, when Y is N and L is —N(R³), R³ is not C₁-C₆-alkylene. In some embodiments, when Y is N and L is —N(R³), R³ is not CH₃. In some embodiments, when Y is N, X is also N. In some embodiments, when X is C(R^{5a}) (e.g., CH), Y is not N. In some embodiments, when Y comprises N, the bond between X and Y is not a double bond. In some embodiments, when Y comprises N, the bond between X and Y is a single bond. In some embodiments, when Y is N, B is not aryl or heteroaryl. In some embodiments, when Y is N, B is not aryl. In some embodiments, when Y is N, B is not heteroaryl. In some embodiments, when Y is N, B is cycloalkyl or heterocyclyl. In some embodiments, when Y is N, B is cycloalkyl. In some embodiments, when Y is N, B is heterocyclyl.

In some embodiments, each of X and Y are independently C optionally substituted with R⁷. In some embodiments, two R⁷ groups are taken together with the atoms to which they are attached to form a cycloalkyl, heterocyclyl, aryl, or heteroaryl. In some embodiments, X and Y are each independently C substituted with R⁷, wherein the two R⁷ groups are taken together with X and Y to form a cycloalkyl, heterocyclyl, aryl, or heteroaryl (e.g., a 3-7 membered cycloalkyl, heterocyclyl, aryl, or heteroaryl). In some embodiments, the R⁷ groups are taken together with X and Y to form a 4-7-membered heterocyclyl. In some embodiments, the R⁷ groups are taken together with X and Y to form a 4-7-membered heteroaryl. In some embodiments, the R⁷ groups are taken together with X and Y to form a 5-membered heterocyclyl. In some embodiments, the R⁷ groups are taken together with X and Y to form a 5-membered heteroaryl. The cycloalkyl, heterocyclyl, aryl, or heteroaryl may be substituted with one or more R⁹.

In some embodiments,

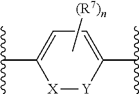

is selected from

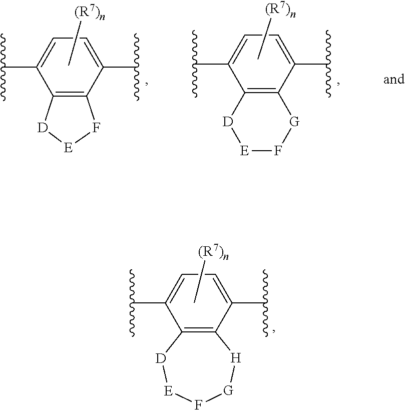

wherein D, E, F, G, and H are each independently C, C(R^{5d}), C(R^{5d})(R^{5e}) N, N(R^{5f}), S, or O; R^{5d}, R^{5e}, and R^{5f} are each independently hydrogen, halo, or C₁-C₆ alkyl, or R^{5d} and R^{5e} are taken together to form an oxo group; R⁷ is as defined herein; and n is an integer of 0, 1, or 2.

In some embodiments, one of D, E, F, G, and H is N(R^{5f}) or N, and the others of D, E, F, G, and H are each independently C(R^{5d}). In some embodiments, two of D, E, F, G, and H are each independently N(R^{5f}) or N, and the others of D, E, F, G, and H are each independently C(R^{5d}). In some embodiments, three of D, E, F, G, and H are each independently N(R^{5f}) or N, and the others of D, E, F, G, and H are each independently C(R^{5d}). In some embodiments, one of D, E, F, G, and H is N(R^{5f}), and the others of D, E, F, G, H are each independently C(R^{5d})(R^{5e}) In some embodiments, two of D, E, F, G, and H are each independently N(R^{5f}), and the others of D, E, F, G, H are each independently C(R^{5d})(R^{5e}). In some embodiments, three of D, E, F, G, and H are each independently N(R^{5f}), and the others of D, E, F, G, H are each independently C(R^{5d})(R^{5e}).

In some embodiments, one of D, E, F, G, and H is N(R^{5f}), and the others of D, E, F, G, and H are independently O or C(R^{5d})(R^{5e}). In some embodiments, one of D, E, F, G, and H is S, and the others of D, E, F, G, and H are each independently N or C(R^{5d}) (e.g., CH). In some embodiments, one of D, E, F, G, and H is S, and the others of D, E, F, G, and H are each independently C(R^{5d}) (e.g. CH).

In some embodiments, one of D, E, and F is N(R^{5f}), and the others of D, E, and F are each independently C(R^{5d}). In some embodiments, two of D, E, and F are each independently N(R^{5f}) or N, and the other of D, E, and F is C(R^{5d}). In some embodiments, three of D, E, and F are each independently N(R^{5f}) or N. In some embodiments, D is N(R^{5f}), and each of E and F are independently C(R^{5d}). In some embodiments, D is N(R^{5f}), E is C(R^{5d}), and F is N. In some embodiments, D is N(R^{5f}), and each of E and F are independently N. In some embodiments, one of D, E, and F is NH, and the others of D, E, and F are independently CH or N.

In some embodiments, one of D, E, and F is N(R^{5f}) (e.g., NH), and the others of D, E, and F are independently O or C(R^{5d})(R^{5e}) (e.g., C(O)). In some embodiments, D is N(R^{5f}) (e.g., CH), E, is C(R^{5d})(R^{5e}) (e.g., C(O)), and F is O. In some embodiments, D is O, E, is C(R^{5d})(R^{5e}) (e.g., C(O)), and F is N(R^{5f}) (e.g., NH). In some embodiments, D is NH, E, is C(O), and F is O. In some embodiments, D is O, E, is C(O), and F is NH.

In some embodiments, one of D, E, and F is S, and the others of D, E, and F, are each independently N or C(R^{5d}) (e.g. CH). In some embodiments, one of D, E, and F is S, and the others of D, E, and F, are each independently C(R^{5d}) (e.g., CH). In some embodiments, D is S, and E and F are each independently N or C(R^{5d}) (e.g., CH). In some embodiments, D is S, E is C(R^{5d}) (e.g., CH), and F is N. In some embodiments, D is S, and E and F are each independently C(R^{5d}) (e.g., CH). In some embodiments, D is S, E is CH, and F is N. In some embodiments, D is S, and E and F are each independently CH.

In some embodiments, one of D, E, and F is N(R^{5f}), and the others of D, E, and F are each independently C(R^{5d})(R^{5e}). In some embodiments, two of D, E, and F are each independently N(R^{5f}), and the other of D, E, and F is C(R^{5d})(R^{5e}). In some embodiments, D is N(R^{5f}), and each of E and F are independently C(R^{5d})(R^{5e}). In some embodiments, E is N(R^{5f}), and each of D and F are independently C(R^{5d})(R^{5e}). In some embodiments, F is N(R^{5f}), and each of D and E are independently C(R^{5d})(R^{5e}). In some embodiments, D is NH, E is C(O), and F is CH₂. In some embodiments, D is CH₂, E is NH, and F is C(O). In some embodiments, D is NH, E is CH, and F is C(O).

In some embodiments,

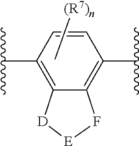

is selected from

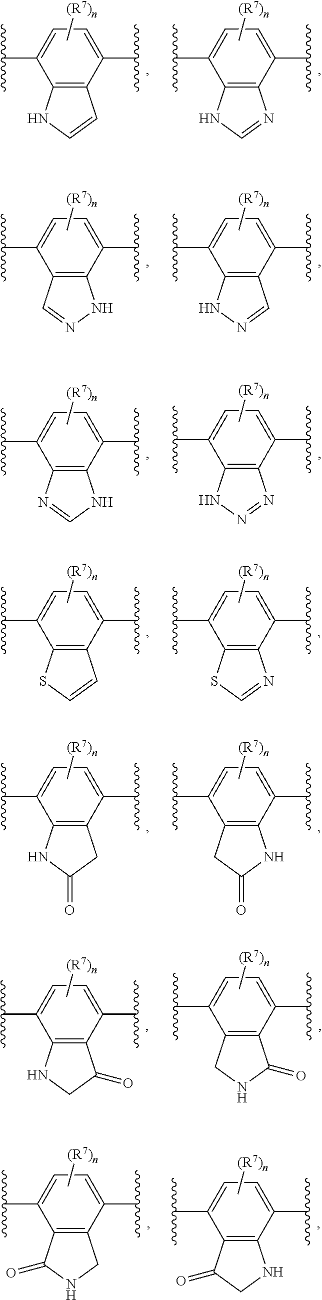

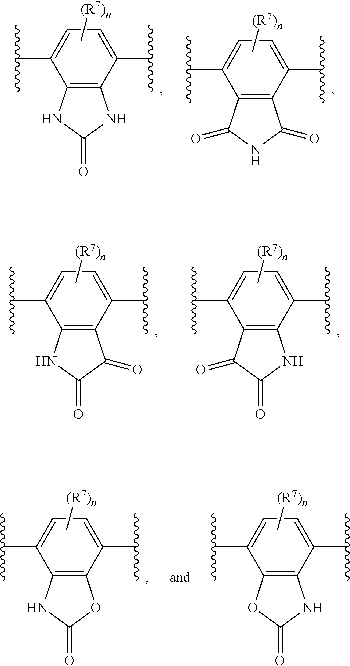

or a tautomer thereof, wherein R⁷ and n are as defined above.

In some embodiments,

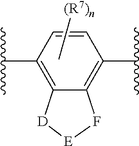

is selected from

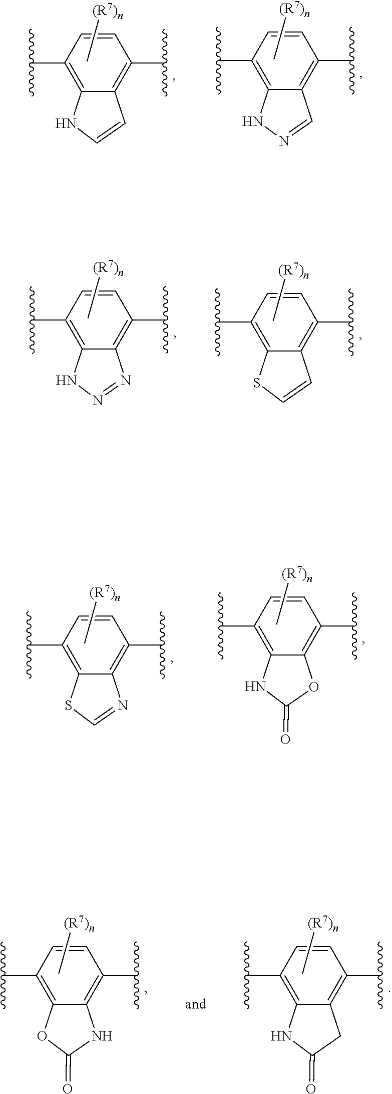

In some embodiments,

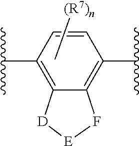

is selected from

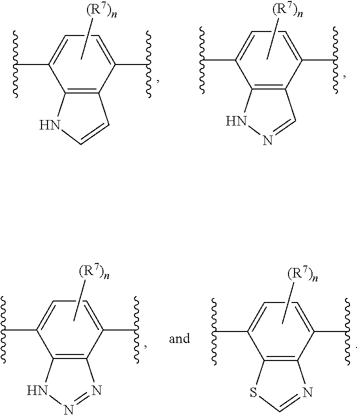

In some embodiments,

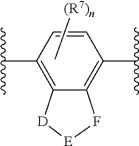

is

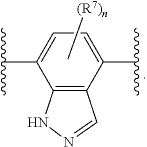

In some embodiments, R¹ is hydrogen. In some embodiments, R¹ is C₁-C₆-alkyl. In some embodiments, R¹ is C₂-C₆-alkenyl. In some embodiments, R¹ is C₂-C₆-alkynyl. In some embodiments, R¹ is C₁-C₆-heteroalkyl. In some embodiments, R¹ is C₁-C₆-haloalkyl (e.g., —CF₃). In some embodiments, R¹ is C₁-alkyl (e.g., methyl). In some embodiments, R¹ is unsubstituted C₁-C₆-alkyl, unsubstituted C₂-C₆-alkenyl, unsubstituted C₂-C₆-alkynyl, unsubstituted C₁-C₆-heteroalkyl, or unsubstituted C₁-C₆-haloalkyl. In some embodiments, R¹ is C₁-C₆-alkyl substituted with one or more R⁸. In some embodiments, R¹ is C₂-C₆-alkenyl substituted with one or more R⁸. In some embodiments, R¹ is C₂-C₆-alkynyl substituted with one or more R⁸. In some embodiments, R¹ is C₁-C₆-heteroalkyl substituted with one or more R⁸. In some embodiments, R¹ is C₁-C₆-haloalkyl substituted with one or more R⁸. In some embodiments, R¹ is methyl.

In some embodiments, R¹ is cycloalkyl (e.g., 3-7 membered cycloalkyl). In some embodiments, R¹ is heterocyclyl (e.g., 3-7 membered heterocyclyl). In some embodiments, R¹ is aryl. In some embodiments, R¹ is C₁-C₆ alkylene-aryl (e.g., benzyl). In some embodiments, R¹ is C₁-C₆ alkenylene-aryl. In some embodiments, R¹ is C₁-C₆ alkylene-heteroaryl. In some embodiments, R¹ is heteroaryl. In some embodiments, R¹ is unsubstituted cycloalkyl, unsubstituted heterocyclyl, unsubstituted aryl, unsubstituted C₁-C₆ alkylene-aryl, unsubstituted C₁-C₆ alkenylene-aryl, unsubstituted C₁-C₆ alkylene-heteroaryl, or unsubstituted heteroaryl. In some embodiments, R¹ is cycloalkyl substituted with one or more R⁸. In some embodiments, R¹ is heterocyclyl substituted with one or more R⁸. In some embodiments, R¹ is aryl substituted with one or more R′. In some embodiments, R¹ is C₁-C₆ alkylene-aryl substituted with one or more R⁸. In some embodiments, R¹ is C₁-C₆ alkenylene-aryl substituted with one or more R⁸. In some embodiments, R¹ is C₁-C₆ alkylene-heteroaryl substituted with one or more R⁸. In some embodiments, R¹ is heteroaryl substituted with one or more R′.

In some embodiments, R¹ is —OR^{A}. In some embodiments, R¹ is —NR^{B}R^{C} (e.g., NH₂ or NMe₂). In some embodiments, R¹ is —NR^{B}C(O)R^{D}. In some embodiments, R¹ is-C(O)NR^{B}R^{C}. In some embodiments, R¹ is —C(O)R^{D}. In some embodiments, R¹ is —C(O)OR^{D}. In some embodiments, R¹ is-SR^{E}. In some embodiments, R¹ is —S(O)ₓR^{D}. In some embodiments, R¹ is halo, e.g., fluoro, chloro, bromo, or iodo. In some embodiments, R¹ is cyano. In some embodiments, R¹ is nitro (—NO₂). In some embodiments, R¹ is oxo.

In some embodiments, two R¹ groups, together with the atoms to which they are attached, form a 3-7-membered cycloalkyl. In some embodiments, two R¹ groups, together with the atoms to which they are attached, form a 3-7-membered heterocyclyl. In some embodiments, two R¹ groups, together with the atoms to which they are attached, form a 5- or 6-membered aryl. In some embodiments, two R¹ groups, together with the atoms to which they are attached, form a 5- or 6-membered heteroaryl. The cycloalkyl, heterocyclyl, aryl, or heteroaryl may be substituted with one or more R⁸.

In some embodiments, R² is hydrogen. In some embodiments, R² is C₁-C₆ alkyl. In some embodiments, R² is C₂-C₆-alkenyl. In some embodiments, R² is C₂-C₆-alkynyl. In some embodiments, R² is C₁-alkyl (e.g., methyl). In some embodiments, R² is methyl. In some embodiments, R² is —OR^{A}. In some embodiments, R² is halo (e.g., fluoro, chloro, bromo, or iodo). In some embodiments, R² is fluoro. In some embodiments, R² is cyano.

In some embodiments, R³ is hydrogen. In some embodiments, R³ is C₁-C₆ alkyl. In some embodiments, R³ is C₁-C₆ haloalkyl. In some embodiments, R³ is C₁-alkyl (e.g., methyl). In some embodiments, R³ is methyl. In some embodiments, R³ is modified with one or more R¹². In some embodiments, R¹² is deuterium. In some embodiments, R³ is CD₃.

In some embodiments, R⁴ is C₁-C₆-alkyl. In some embodiments, R⁴ is C₁-C₆-heteroalkyl. In some embodiments, R⁴ is C₁-C₆-haloalkyl. In some embodiments, R⁴ is cycloalkyl. In some embodiments, R⁴ is halo (e.g., fluoro, chloro, bromo, or iodo). In some embodiments, R⁴ is cyano. In some embodiments, R⁴ is oxo. In some embodiments, R⁴ is —OR^{A}. In some embodiments, R⁴ is —NR^{B}R^{C}. In some embodiments, R⁴ is —C(O)R^{D} or —C(O)OR^{D}.

In some embodiments, R^{5a} and R^{5b} are each independently hydrogen or C₁-C₆-alkyl. In some embodiments, R^{5a} is hydrogen. In some embodiments, R^{5b} is hydrogen. In some embodiments, R^{5a} and R^{5b} are taken together to form an oxo group. In some embodiments, R^{5c} is hydrogen. In some embodiments, R^{5c} is C₁-C₆-alkyl. In some embodiments, R⁵, is C₁-C₆-haloalkyl (e.g., —CF₃ or —CHF₂). In some embodiments, R^{5c} is —CF₃. In some embodiments, R^{5c} is —CHF₂. In some embodiments, R^{5c} is —C(O)R^{D} (e.g., —C(O)CH₃). In some embodiments, R^{5c} is —C(O)CH₃).

In some embodiments, R^{5d}, R^{5e}, and R^{5f} are each independently hydrogen, C₁-C₆-alkyl, halo, or R^{5d}, R^{5e} are taken together to form an oxo group. In some embodiments, R^{5d} is hydrogen. In some embodiments, R^{5e} is hydrogen. In some embodiments, R^{5f} is hydrogen. In some embodiments, R^{5e} and R^{5f}, are together to form an oxo group.

In some embodiments, R⁷ is C₁-C₆-alkyl. In some embodiments, R⁷ is C₂-C₆-alkenyl. In some embodiments, R⁷ is C₂-C₆-alkynyl. In some embodiments, R⁷ is C₁-C₆-heteroalkyl. In some embodiments, R⁷ is C₁-C₆-haloalkyl. In some embodiments, R⁷ is unsubstituted C₁-C₆-alkyl, unsubstituted C₂-C₆-alkenyl, unsubstituted C₂-C₆-alkynyl, unsubstituted C₁-C₆-heteroalkyl, or unsubstituted C₁-C₆-haloalkyl. In some embodiments, R⁷ is C₁-C₆-alkyl substituted with one or more R⁹. In some embodiments, R⁷ is C₂-C₆-alkenyl substituted with one or more R⁹. In some embodiments, R⁷ is C₂-C₆-alkynyl substituted with one or more R⁹. In some embodiments, R⁷ is C₁-C₆-heteroalkyl substituted with one or more R⁹. In some embodiments, R⁷ is C₁-C₆-haloalkyl substituted with one or more R⁹. In some embodiments, R⁷ is halo, e.g., fluoro, chloro, bromo, or iodo. In some embodiments, R⁷ is fluoro. In some embodiments, R⁷ is cyano. In some embodiments, R⁷ is oxo. In some embodiments, R⁷ is NR^{B}C(O)R^{D}. In some embodiments, R⁷ is —C(O)NR^{B}R^{C}. In some embodiments, R⁷ is —C(O)R^{D}. In some embodiments, R⁷ is —SR^{E}.

In some embodiments, two R⁷ groups, together with the atoms to which they are attached (e.g., X or Y) form a 4-7-membered cycloalkyl. In some embodiments, two R⁷ groups, together with the atoms to which they are attached (e.g., X or Y) form a 4-7-membered heterocyclyl. In some embodiments, two R⁷ groups, together with the atoms to which they are attached (e.g., X or Y) form a 5- or 6-membered aryl. In some embodiments, two R⁷ groups, together with the atoms to which they are attached (e.g., X or Y) form a 5- or 6-membered heteroaryl. In some embodiments, two R⁷ groups, together with the atoms to which they are attached (e.g., X or Y), form a 5-membered heterocyclyl. In some embodiments, two R⁷ groups, together with the atoms to which they are attached (e.g., X or Y), form a 5-membered heteroaryl. The cycloalkyl, heterocyclyl, aryl, or heteroaryl may be substituted with one or more R⁹.

In some embodiments, R⁸ is C₁-C₆-alkyl. In some embodiments, R⁸ is C₂-C₆-alkenyl. In some embodiments, R⁸ is C₂-C₆-alkynyl. In some embodiments, R⁸ is C₁-C₆-heteroalkyl. In some embodiments, R⁸ is C₁-C₆-haloalkyl. In some embodiments, R⁸ is unsubstituted C₁-C₆-alkyl, unsubstituted C₂-C₆-alkenyl, unsubstituted C₂-C₆-alkynyl, unsubstituted C₁-C₆-haloalkyl, or unsubstituted C₁-C₆-heteroalkyl. In some embodiments, R⁸ is C₁-C₆-alkyl substituted with one or more R¹¹. In some embodiments, R⁸ is C₂-C₆-alkenyl substituted with one or more R¹¹. In some embodiments, R⁸ is C₂-C₆-alkynyl substituted with one or more R¹¹. In some embodiments, R⁸ is C₁-C₆-haloalkyl substituted with one or more R¹¹. In some embodiments, R⁸ is C₁-C₆-heteroalkyl substituted with one or more R¹¹.

In some embodiments, R⁸ is cycloalkyl. In some embodiments, R⁸ is heterocyclyl. In some embodiments, R⁸ is aryl. In some embodiments, R⁸ is heteroaryl. In some embodiments, R⁸ is unsubstituted cycloalkyl, unsubstituted heterocyclyl, unsubstituted aryl, or unsubstituted heteroaryl. In some embodiments, R⁸ is cycloalkyl substituted with one or more R¹¹. In some embodiments, R⁸ is heterocyclyl substituted with one or more R¹¹. In some embodiments, R⁸ is aryl substituted with one or more R¹¹. In some embodiments, R⁸ is heteroaryl substituted with one or more R¹¹.

In some embodiments, R⁸ is halo (e.g., fluoro, chloro, bromo, or iodo). In some embodiments, R⁸ is cyano. In some embodiments, R⁸ is oxo. In some embodiments, R⁸ is —OR^{A}. In some embodiments, R⁸ is —NR^{B}R^{C}. In some embodiments, R⁸ is —NR^{B}C(O)R^{D}. In some embodiments, R⁸ is —NO₂. In some embodiments, R⁸ is —C(O)NR^{B}R^{C}. In some embodiments, R⁸ is —C(O)R^{D}. In some embodiments, R⁸ is —C(O)OR^{D}. In some embodiments, R⁸ is —SR^{E}. In some embodiments, R⁸ is —S(O)R^{D}.

In some embodiments, R⁹ is C₁-C₆-alkyl. In some embodiments, R⁹ is C₂-C₆-alkenyl. In some embodiments, R⁹ is C₂-C₆-alkynyl. In some embodiments, R⁹ is C₁-C₆-heteroalkyl. In some embodiments, R⁹ is C₁-C₆-haloalkyl. In some embodiments, R⁹ is unsubstituted C₁-C₆-alkyl, unsubstituted C₂-C₆-alkenyl, unsubstituted C₂-C₆-alkynyl, unsubstituted C₁-C₆-haloalkyl, or unsubstituted C₁-C₆-heteroalkyl. In some embodiments, R⁹ is C₁-C₆-alkyl substituted with one or more R¹¹. In some embodiments, R⁹ is C₂-C₆-alkenyl substituted with one or more R¹¹. In some embodiments, R⁹ is C₂-C₆-alkynyl substituted with one or more R¹¹. In some embodiments, R⁹ is C₁-C₆-haloalkyl substituted with one or more R¹¹. In some embodiments, R⁹ is C₁-C₆-heteroalkyl substituted with one or more R¹¹.

In some embodiments, R⁹ is cycloalkyl. In some embodiments, R⁹ is heterocyclyl. In some embodiments, R⁹ is aryl. In some embodiments, R⁹ is heteroaryl. In some embodiments, R⁹ is unsubstituted cycloalkyl, unsubstituted heterocyclyl, unsubstituted aryl, or unsubstituted heteroaryl. In some embodiments, R⁹ is cycloalkyl substituted with one or more R¹¹. In some embodiments, R⁹ is heterocyclyl substituted with one or more R¹¹. In some embodiments, R⁹ is aryl substituted with one or more R¹¹. In some embodiments, R⁹ is heteroaryl substituted with one or more R¹¹.

In some embodiments, R⁹ is halo (e.g., fluoro, chloro, bromo, or iodo). In some embodiments, R⁹ is cyano. In some embodiments, R⁹ is oxo. In some embodiments, R⁹ is —OR^{A}. In some embodiments, R⁹ is —NR^{B}R^{C}. In some embodiments, R⁹ is —NR^{B}C(O)R^{D}. In some embodiments, R⁹ is —NO₂. In some embodiments, R⁹ is —C(O)NR^{B}R^{C}. In some embodiments, R⁹ is —C(O)R^{D}. In some embodiments, R⁹ is —C(O)OR^{D}. In some embodiments, R⁹ is —SR^{E}. In some embodiments, R⁹ is —S(O)ₓR^{D}.

In some embodiments, R¹⁰ is C₁-C₆-alkyl. In some embodiments, R¹⁰ is halo (e.g., fluoro, chloro, bromo, or iodo).

In some embodiments, R¹¹ is C₁-C₆-alkyl. In some embodiments, R¹¹ is C₁-C₆-heteroalkyl. In some embodiments, R¹¹ is C₁-C₆-haloalkyl (e.g., —CF₃). In some embodiments, R¹¹ is cycloalkyl. In some embodiments, R¹¹ is heterocyclyl. In some embodiments, R¹¹ is aryl. In some embodiments, R¹¹ is heteroaryl. In some embodiments, R¹¹ is halo. In some embodiments, R¹¹ is cyano. In some embodiments, R¹¹ is oxo. In some embodiments, R¹¹ is —OR^{A}.

In some embodiments, R^{A} is hydrogen. In some embodiments, R^{A} is C₁-C₆ alkyl (e.g., methyl). In some embodiments, R^{A} is C₁-C₆ haloalkyl. In some embodiments, R^{A} is aryl. In some embodiments, R^{A} is heteroaryl. In some embodiments, R^{A} is C₁-C₆ alkylene-aryl (e.g., benzyl). In some embodiments, R^{A} is C₁-C₆ alkylene-heteroaryl. In some embodiments, R^{A} is C(O)R^{D}. In some embodiments, R^{A} is —S(O)ₓR^{D}.

In some embodiments, R^{B}, R^{C}, or both are each independently hydrogen, C₁-C₆-alkyl, C₁-C₆-heteroalkyl, cycloalkyl, heterocyclyl, or —OR^{A}. In some embodiments, each of R^{B} and R^{C} is independently hydrogen. In some embodiments, each of R^{B} and R^{C} is independently C₁-C₆ alkyl. In some embodiments, one of R^{B} and R^{C} is hydrogen, and the other of R^{B} and R^{C} is C₁-C₆ alkyl. In some embodiments, R^{B} and R^{C} together with the atom to which they are attached form a 3-7-membered heterocyclyl ring optionally substituted with one or more of R¹⁰ (e.g., 1, 2, or 3 R¹⁰).

In some embodiments, R^{D}, R^{E}, or both are each independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl (e.g., benzyl), or C₁-C₆ alkylene-heteroaryl. In some embodiments, each of R^{D} and R^{E} is independently hydrogen. In some embodiments, each of R^{D} and R^{E} is independently C₁-C₆ alkyl. In some embodiments, R^{D} is hydrogen. In some embodiments, R^{E} is hydrogen. In some embodiments, R^{D} is C₁-C₆ alkyl (e.g., methyl). In some embodiments, R^{E} is C₁-C₆ alkyl (e.g., methyl). In some embodiments, R^{D} is C₁-C₆ heteroalkyl. In some embodiments, R^{E} is C₁-C₆ heteroalkyl. In some embodiments, R^{D} is C₁-C₆ haloalkyl. In some embodiments, R^{E} is C₁-C₆ haloalkyl. In some embodiments, R^{D} is cycloalkyl. In some embodiments, R^{E} is cycloalkyl. In some embodiments, R^{D} is heterocyclyl. In some embodiments, R^{E} is heterocyclyl. In some embodiments, R^{D} is aryl. In some embodiments, R^{E} is aryl. In some embodiments, R^{D} is heteroaryl. In some embodiments, R^{E} is heteroaryl. In some embodiments, R^{D} is C₁-C₆ alkylene-aryl (e.g., benzyl). In some embodiments, R^{E} is C₁-C₆ alkylene-aryl (e.g., benzyl). In some embodiments, R^{D} is C₁-C₆ alkylene-heteroaryl. In some embodiments, R^{E} is C₁-C₆ alkylene-heteroaryl.

In some embodiments, m is an integer between 0 and 2 (e.g., 0, 1, or 2). In some embodiments, m is 0. In some embodiments, m is 1. In some embodiments, m is 2. In some embodiments, n is an integer between 0 and 4 (e.g., 0, 1, 2, 3, or 4). In some embodiments, n is 0. In some embodiments, n is 1. In some embodiments, n is 2. In some embodiments, n is 3. In some embodiments, n is 4. In some embodiments, x is an integer between 0 and 2 (e.g., 0, 1, or 2). In some embodiments, x is 0. In some embodiments, x is 1. In some embodiments, x is 2.

In some embodiments, the compound is a compound of Formula (I-b):

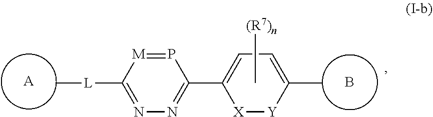

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —C(O)—, —N(R³)—, —S(O)ₓ—, —N(R³)C(O)—, or —C(O)N(R³)—, wherein each alkylene and heteroalkylene is optionally substituted with one or more R⁴; M and P are each independently C(R²) or N; X and Y are each independently C, C(R^{5a}) C(R^{5a})(R^{5b}), N, or N(R^{5c}), wherein the bond between X and Y may be a single or double bond as valency permits, and wherein X and Y may not both be C(R^{5a})(R^{5b}); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}—NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R′; each R² is independently hydrogen, halo, cyano, C₁-C₆-alkyl, C₂-C₆-alkenyl, C₂-C₆-alkynyl, or —OR^{A}; each R³ is independently hydrogen, C₁-C₆-alkyl, or C₁-C₆-haloalkyl; each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R^{5a} is hydrogen, C₁-C₆-alkyl, or —ORF; R^{5b} is hydrogen or C₁-C₆-alkyl; or R^{5a} and R^{5b}, together with the carbon atom to which they are attached, form an oxo group; each R^{5c} is hydrogen, C₁-C₆-alkyl, C₁-C₆-haloalkyl, or C(O)R^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹; or two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E} or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; RF is hydrogen or C₁-C₆ alkyl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; n is 0, 1, 2, 3, or 4; and x is 0, 1, or 2.

In some embodiments, the present disclosure features a compound of Formula (I-c):

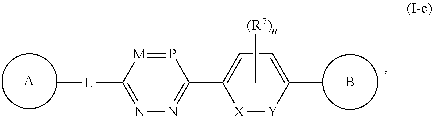

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; M and P are each independently C(R²) or N; X and Y are each independently C, C(R^{5a}), C(R^{5a})(R^{5b}), N, or N(R^{5c}), wherein the bond between X and Y may be a single or double bond as valency permits, and wherein X and Y may not both be C(R^{5a})(R^{5b}); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R′; each R² is independently hydrogen, halo, cyano, C₁-C₆-alkyl, C₂-C₆-alkenyl, C₂-C₆-alkynyl, or —OR^{A}; each R³ is independently hydrogen, C₁-C₆-alkyl, or C₁-C₆-haloalkyl; each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, C(O)R^{D}, or —C(O)OR^{D}; R⁵, is hydrogen, C₁-C₆-alkyl, or —ORF; R^{5b} is hydrogen or C₁-C₆-alkyl; or R^{5a} and R^{5b}, together with the carbon atom to which they are attached, form an oxo group; each R^{5c} is hydrogen, C₁-C₆-alkyl, C₁-C₆-haloalkyl, or C(O)R^{D}; each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹; or two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹; R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆-heteroalkyl, cycloalkyl, heterocyclyl, —OR^{A}; or R^{B} and R^{C} together with the atom to which they are attached form a 3-7-membered heterocyclyl ring optionally substituted with one or more R¹⁰; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; RF is hydrogen or C₁-C₆-alkyl; R¹⁰ is C₁-C₆-alkyl or halo; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; n is 0, 1, 2, 3, or 4; and x is 0, 1, or 2.

In some embodiments, the compound of Formula (I) is a compound of Formula (I-d):

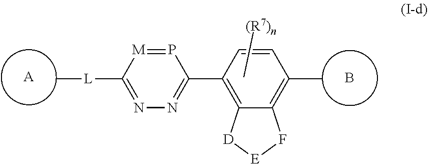

or a pharmaceutically acceptable salt, solvate, hydrate, tautomer, or stereoisomer thereof, wherein:

A and B are each independently cycloalkyl, heterocyclyl, aryl, or heteroaryl, each of which is optionally substituted with one or more R¹;

L is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴;

M and P are each independently C(R²) or N;

D, E, and F are each independently C(R^{5d}), C(R^{5d})(R^{5e}), N, N(R^{5f}), S, or O, wherein the bonds between the atoms in the ring comprising D, E, and F may be a single bonds or double bonds as valency permits;

each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or

two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁸;

each R² is independently hydrogen, halo, cyano, C₁-C₆-alkyl, C₂-C₆-alkenyl, C₂-C₆-alkynyl, or —OR^{A};

each R³ is independently hydrogen, C₁-C₆-alkyl, C₁-C₆-heteroalkyl, C₁-C₆-haloalkyl, cycloalkyl or heterocyclyl, wherein each alkyl, heteroalkyl, haloalkyl, cycloalkyl, and heterocyclyl is optionally substituted with one or more R¹²;

each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};

R^{5d} and R^{5e} are each independently hydrogen, halo, or C₁-C₆ alkyl; or

R^{5d} and R^{5e} are taken together to form an oxo group;

R^{5f} is hydrogen, halo, or C₁-C₆ alkyl;

each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹ or

two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹;

R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;

each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};

each of R^{B} and R^{C} is independently hydrogen, C₁-C₆ alkyl, C₁-C₆-heteroalkyl, cycloalkyl, heterocyclyl, —OR^{A}; or

R^{B} and R^{C} together with the atom to which they are attached form a 3-7-membered heterocyclyl ring optionally substituted with one or more R¹⁰;

each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;

each R¹⁰ is independently C₁-C₆-alkyl or halo;

each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

n is 0, 1, 2, 3, or 4; and

x is 0, 1, or 2.

In some embodiments, A is selected from

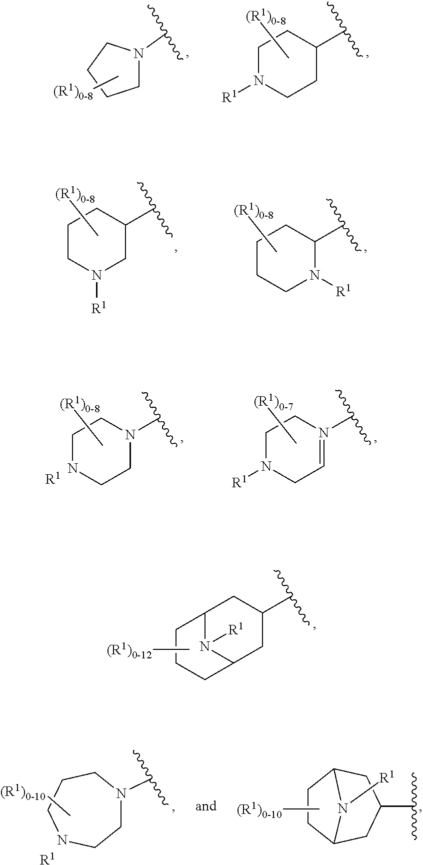

wherein R¹ is as defined herein.

In some embodiments, A is selected from

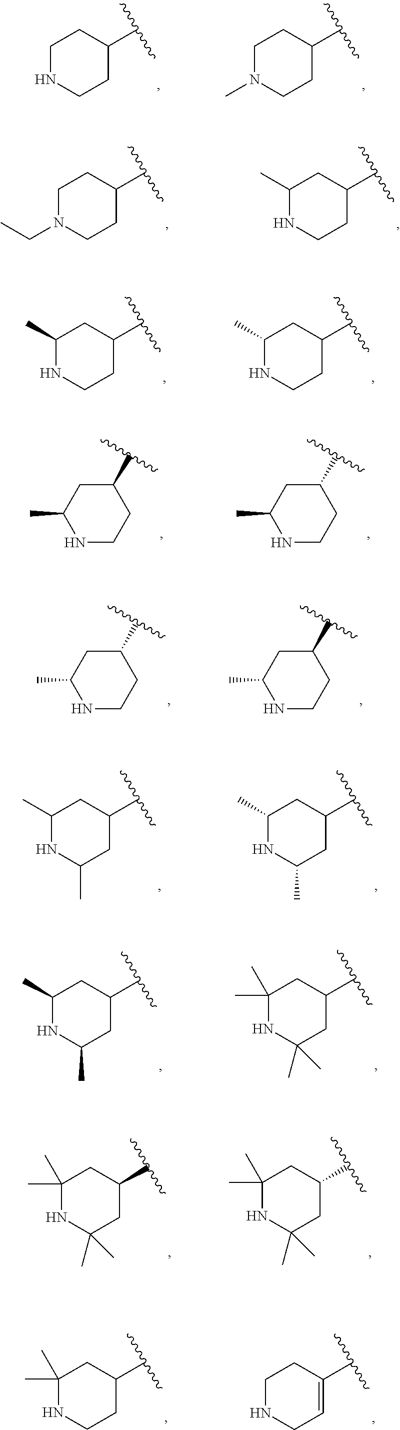

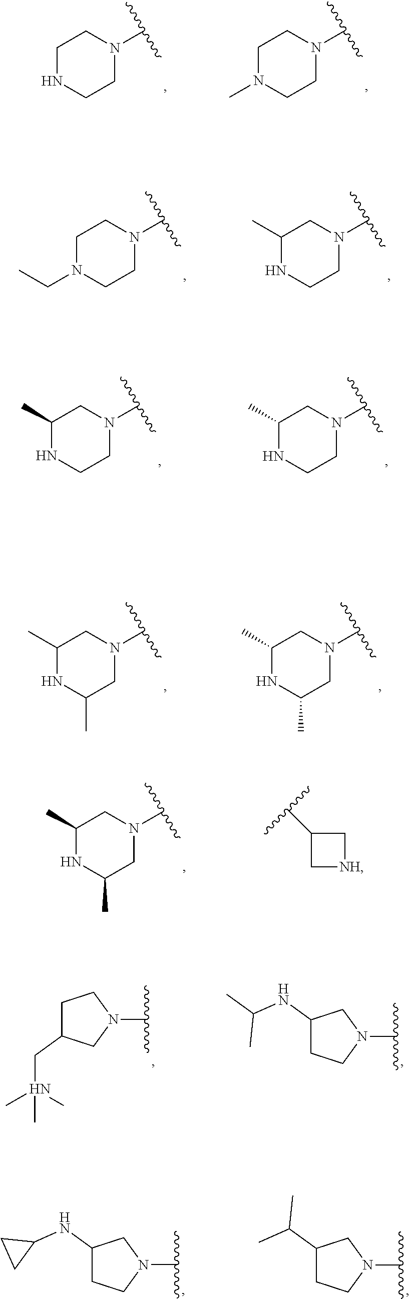

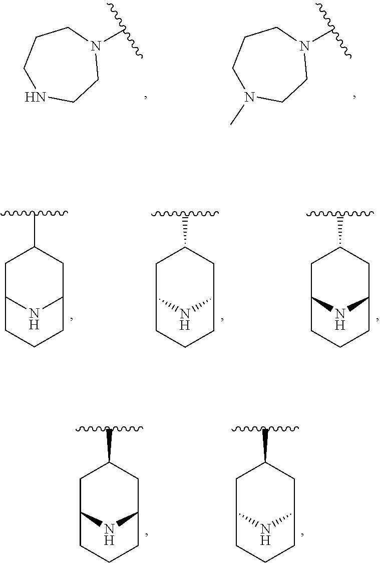

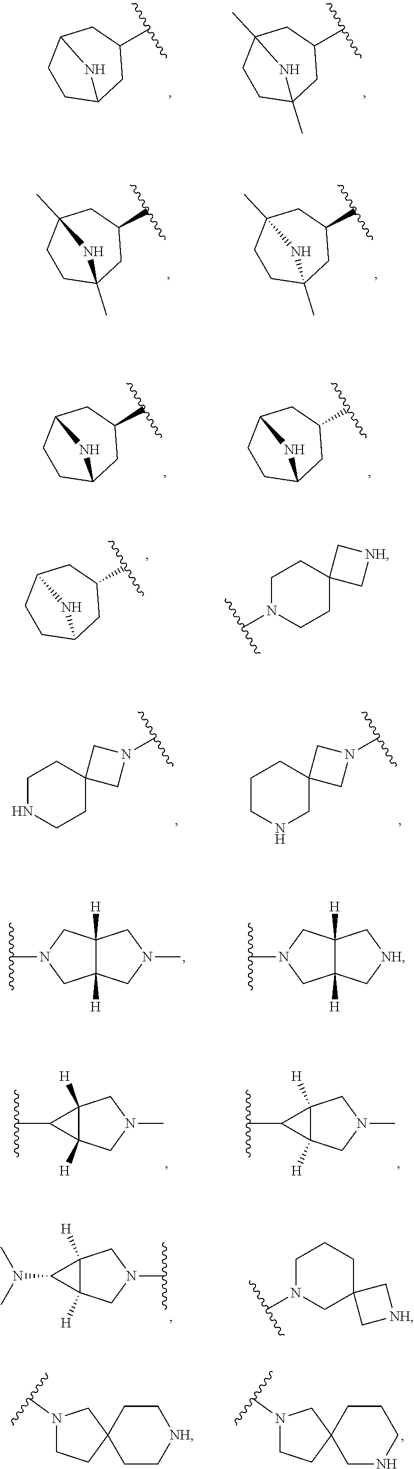

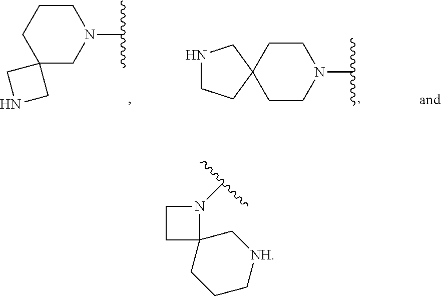 [SMILES: *N1CCCC2(CNC2)C1 ; *N1CCC2(CCNC2)CC1 ; *N1CCC12CCCNC2]

In some embodiments, A is selected from

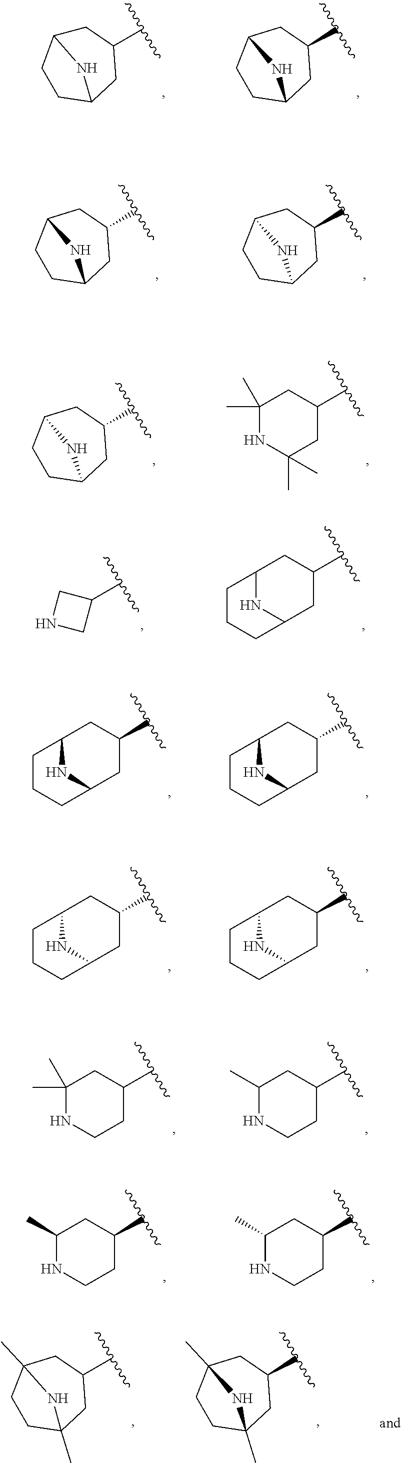

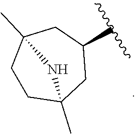 [SMILES: *[C@@H]1C[C@]2(C)CC[C@](C)(C1)N2]

In some embodiments, A is selected from

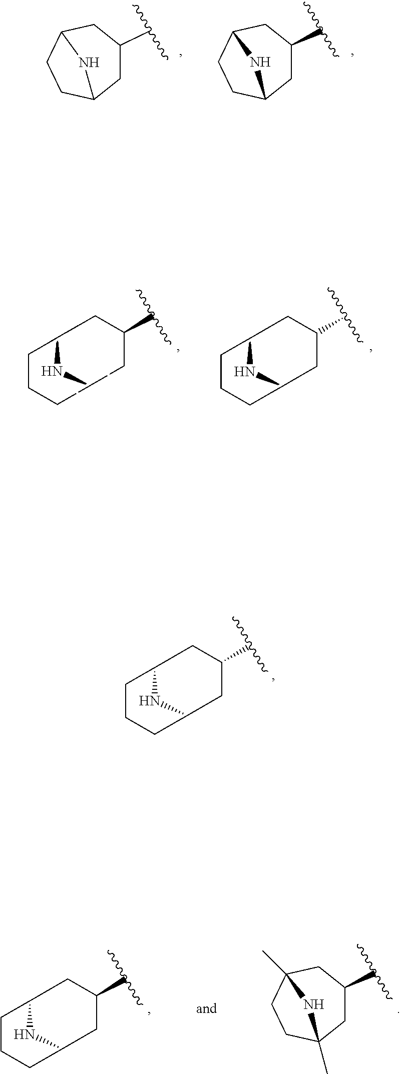

In some embodiments, A is

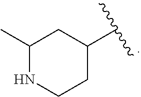 [SMILES: *C1CCNC(C)C1]

In some embodiments, A is

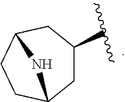 [SMILES: *[C@@H]1C[C@H]2CC[C@@H](C1)N2]

In some embodiments, A is

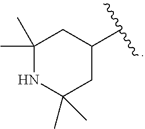 [SMILES: *C1CC(C)(C)NC(C)(C)C1]

In some embodiments, A is

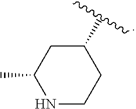 [SMILES: *[C@@H]1CCN[C@H](C)C1]

In some embodiments, A is

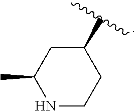 [SMILES: *[C@H]1CCN[C@@H](C)C1]

In some embodiments, A is

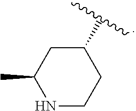 [SMILES: *[C@@H]1CCN[C@@H](C)C1]

In some embodiments, A is

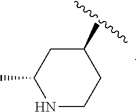

In some embodiments, A is

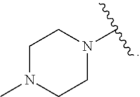 [SMILES: *N1CCN(C)CC1]

In some embodiments, A is

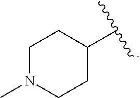 [SMILES: *C1CCN(C)CC1]

In some embodiments, A is

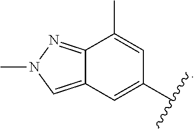 [SMILES: *c1cc(C)c2nn(C)cc2c1]

In some embodiments, A is

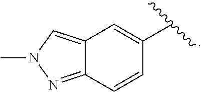 [SMILES: *c1ccc2nn(C)cc2c1]

In some embodiments, B is selected from

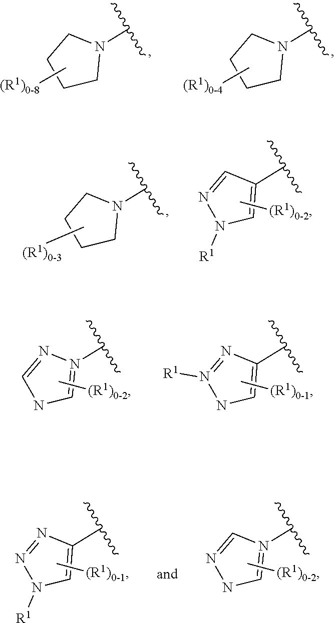

wherein R¹ is as defined herein. In some embodiments, B is selected from

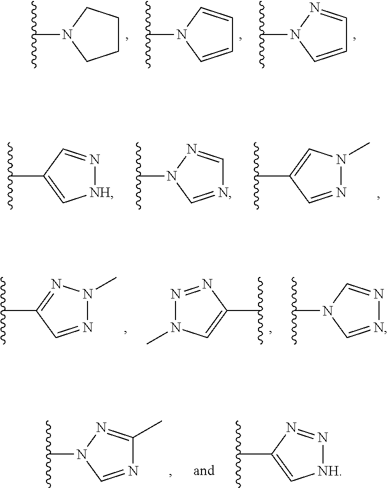

In some embodiments, B is

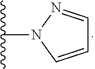 [SMILES: *n1cccn1]

In some embodiments B is

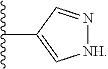 [SMILES: *c1cn[nH]c1]

In some embodiments, B is

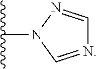 [SMILES: *n1cncn1]

In some embodiments, B is

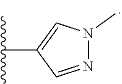 [SMILES: *c1cnn(C)c1]

In some embodiments, B is

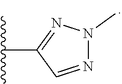 [SMILES: *c1cnn(C)n1]

In some embodiments, B is

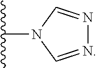 [SMILES: *n1cnnc1]

In some embodiments, B is

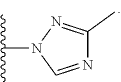 [SMILES: *n1cnc(C)n1]

In some embodiments, B is

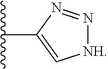 [SMILES: *c1c[nH]nn1]

In some embodiments, one of D, E, and F is N(R^{5f}), and the others of D, E, and F are each independently C(R^{5d}). In some embodiments, two of D, E, and F are each independently N(R^{5f}) or N, and the other of D, E, and F is C(R^{5d}). In some embodiments, three of D, E, and F are each independently N(R^{5f}) or N. In some embodiments, D is N(R^{5f}), and each of E and F are independently C(R^{5d}). In some embodiments, D is N(R^{5f}), E is C(R^{5d}), and F is N. In some embodiments, D is N(R^{5f}), and each of E and F are independently N. In some embodiments, one of D, E, and F is NH, and the others of D, E, and F are independently CH or N.

In some embodiments, one of D, E, and F is N(R^{5f}) (e.g., NH), and the others of D, E, and F are independently O or C(R^{5d})(R^{5e}) (e.g., C(O)). In some embodiments, D is N(R^{5f}) (e.g., CH), E, is C(R^{5d})(R^{5e}) (e.g., C(O)), and F is O. In some embodiments, D is O, E, is C(R^{5d})(R^{5e}) (e.g., C(O)), and F is N(R^{5f}) (e.g., NH). In some embodiments, D is NH, E, is C(O), and F is O. In some embodiments, D is O, E, is C(O), and F is NH.

In some embodiments, one of D, E, and F is S, and the others of D, E, and F, are each independently N or C(R^{5d}) (e.g. CH). In some embodiments, one of D, E, and F is S, and the others of D, E, and F, are each independently C(R^{5d}) (e.g., CH). In some embodiments, D is S, and E and F are each independently N or C(R^{5d}) (e.g., CH). In some embodiments, D is S, E is C(R^{5d}) (e.g., CH), and F is N. In some embodiments, D is S, and E and F are each independently C(R^{5d}) (e.g., CH). In some embodiments, D is S, E is CH, and F is N. In some embodiments, D is S, and E and F are each independently CH.

In some embodiments, one of D, E, and F is N(R^{5f}), and the others of D, E, and F are each independently C(R^{5d})(R^{5e}). In some embodiments, two of D, E, and F are each independently N(R^{5f}), and the other of D, E, and F is C(R^{5d})(R^{5e}). In some embodiments, D is N(R^{5f}), and each of E and F are independently C(R^{5d})(R^{5e}). In some embodiments, E is N(R^{5f}), and each of D and F are independently C(R^{5d})(R^{5e}). In some embodiments, F is N(R^{5f}), and each of D and E are independently C(R^{5d})(R^{5e}). In some embodiments, D is NH, E is C(O), and F is CH₂. In some embodiments, D is CH₂, E is NH, and F is C(O). In some embodiments, D is NH, E is CH, and F is C(O).

In some embodiments,

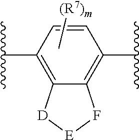

is selected from

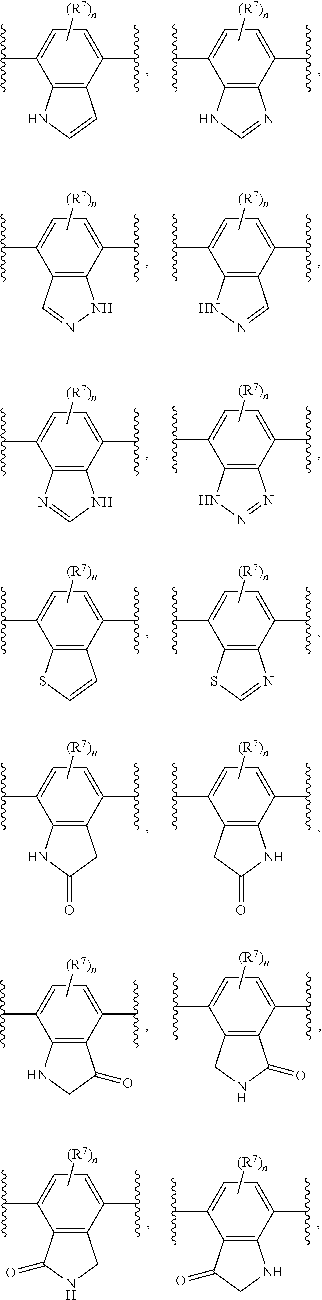

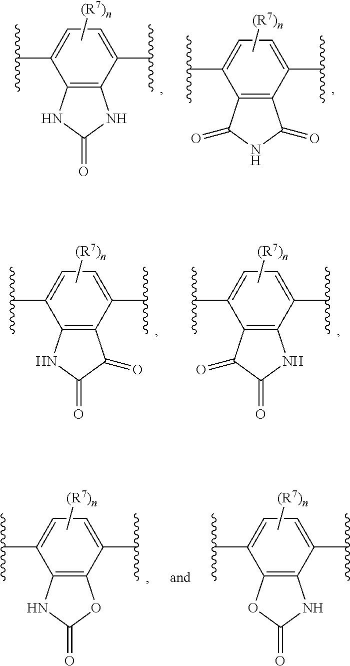

or a tautomer thereof, wherein R⁷ and n are as defined above.

In some embodiments,

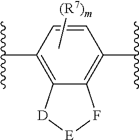

is selected from N

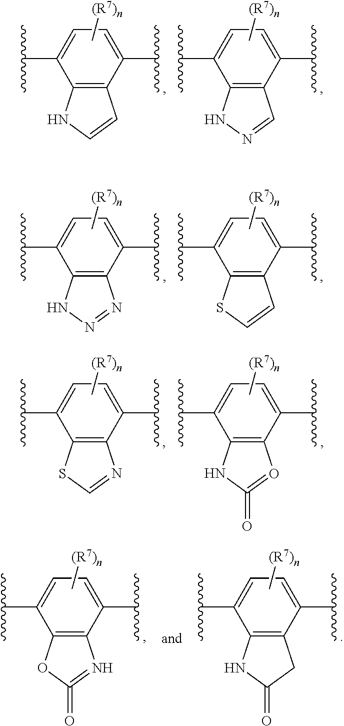

In some embodiments,

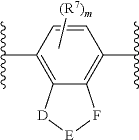

is selected from

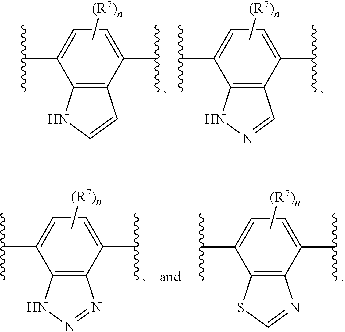

In some embodiments,

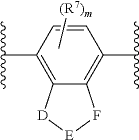

is

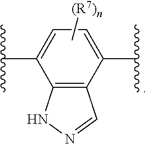

In some embodiments, the compound of Formula (I) is a compound of Formula (I-e):

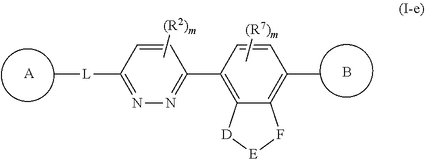

or a pharmaceutically acceptable salt, solvate, hydrate, tautomer, or stereoisomer thereof, wherein:

A and B are each independently cycloalkyl, heterocyclyl, aryl, or heteroaryl, each of which is optionally substituted with one or more R¹;

L is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴;

D, E, and F are each independently C(R^{5d}), C(R^{5d})(R^{5e}), N, N(R^{5f}), S, or O, wherein the bonds between the atoms in the ring comprising D, E, and F may be a single bonds or double bonds as valency permits;

each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or

two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R′;

each R² is independently hydrogen, halo, cyano, C₁-C₆-alkyl, C₂-C₆-alkenyl, C₂-C₆-alkynyl, or —OR^{A};

each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R²;

each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};

R^{5d} and R^{5c} are each independently hydrogen, halo, or C₁-C₆ alkyl; or

R^{5d} and R^{5c} are taken together to form an oxo group;

R^{5f} is hydrogen, halo, or C₁-C₆ alkyl;

each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹ or

two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹;

R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;

each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};

each of R^{B} and R^{C} is independently hydrogen, C₁-C₆ alkyl, C₁-C₆-heteroalkyl, cycloalkyl, heterocyclyl, —OR^{A}; or

R^{B} and R^{C} together with the atom to which they are attached form a 3-7-membered heterocyclyl ring optionally substituted with one or more R¹⁰;

each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;

each R¹⁰ is independently C₁-C₆-alkyl or halo;

each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

m is 0, 1, or 2

n is 0, 1, 2, 3, or 4; and

x is 0, 1, or 2.

In some embodiments, A is selected from

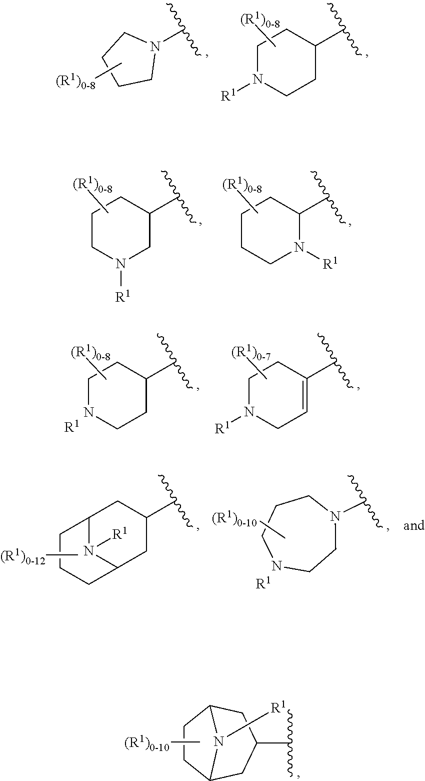

wherein R¹ is as defined herein.

In some embodiments, A is selected from

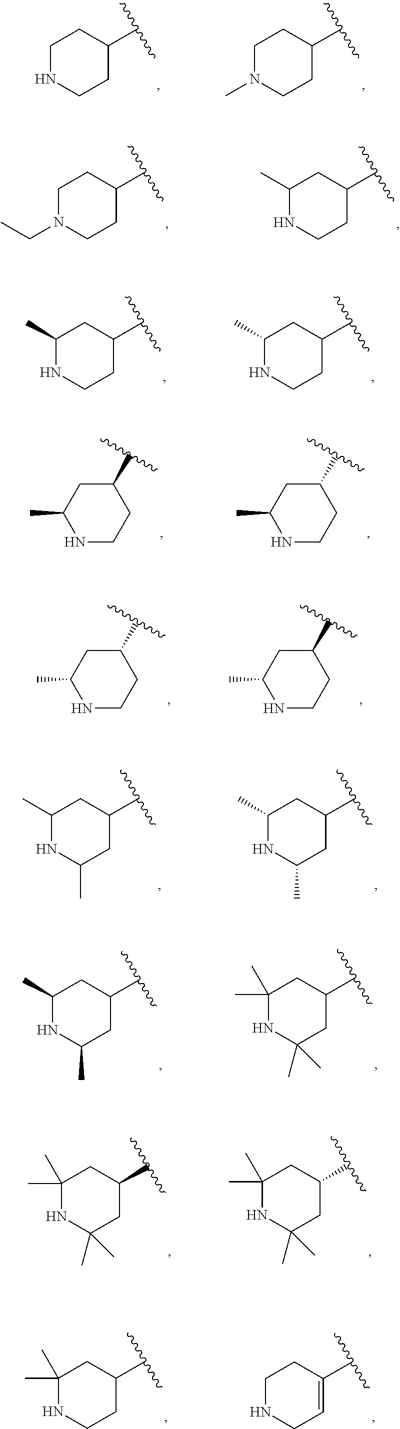

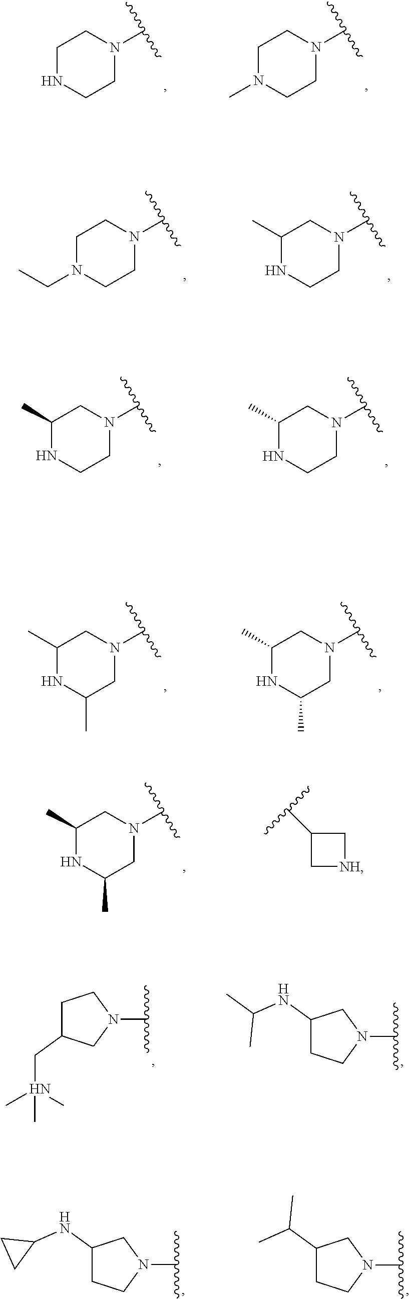

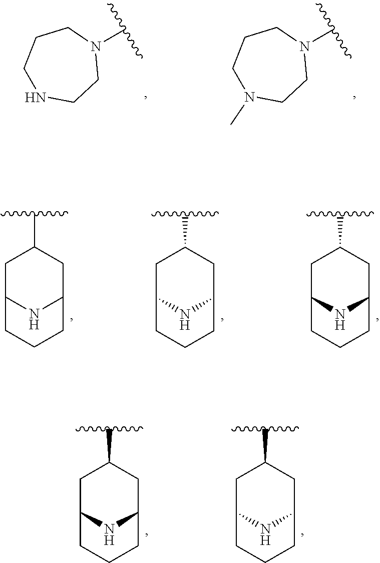

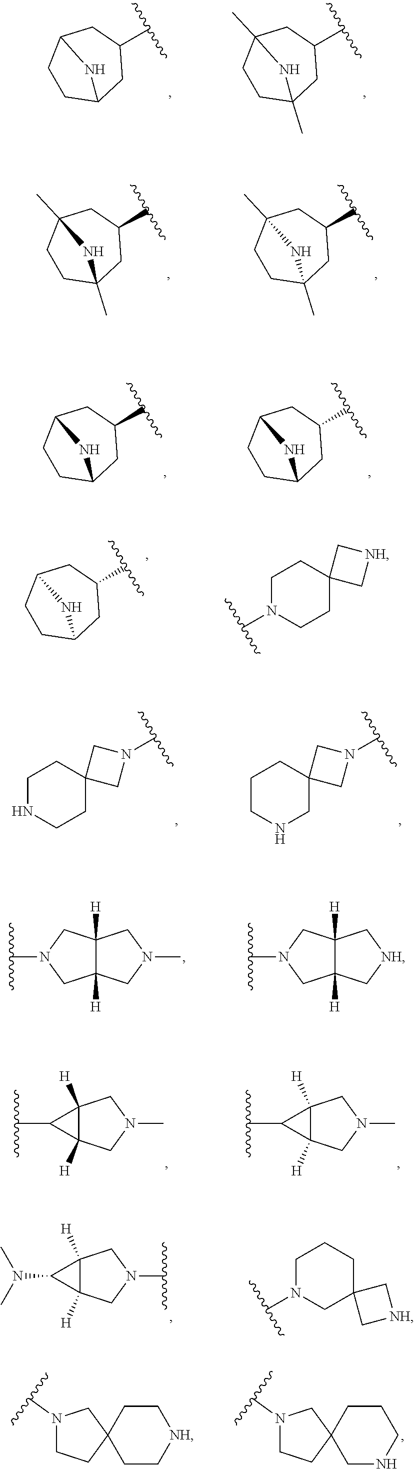

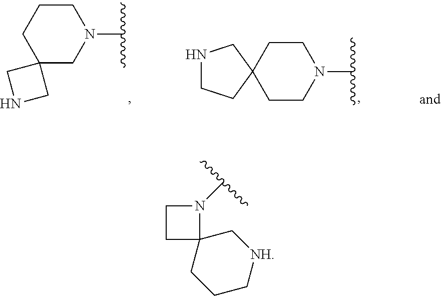 [SMILES: *N1CCCC2(CNC2)C1 ; *N1CCC2(CCNC2)CC1 ; *N1CCC12CCCNC2]

In some embodiments, A is selected from

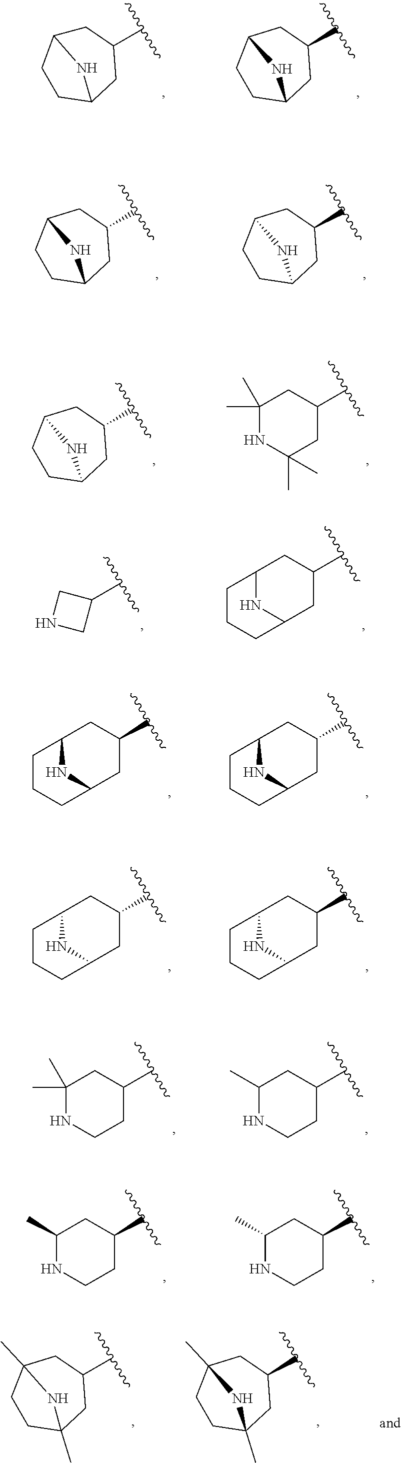

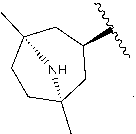 [SMILES: *[C@@H]1C[C@]2(C)CC[C@](C)(C1)N2]

In some embodiments, A is selected from

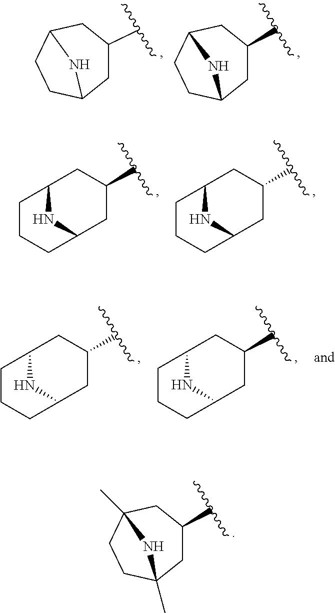

In some embodiments, A is

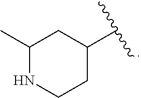 [SMILES: *C1CCNC(C)C1]

In some embodiments, A is

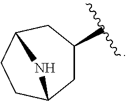 [SMILES: *[C@@H]1C[C@H]2CC[C@@H](C1)N2]

In some embodiments, A is

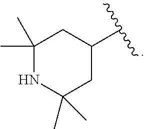 [SMILES: *C1CC(C)(C)NC(C)(C)C1]

In some embodiments, A is

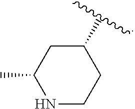 [SMILES: *[C@@H]1CCN[C@H](C)C1]

In some embodiments, A is

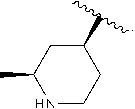 [SMILES: *[C@H]1CCN[C@@H](C)C1]

In some embodiments, A is

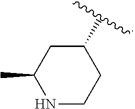 [SMILES: *[C@@H]1CCN[C@@H](C)C1]

In some embodiments, A is

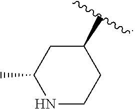 [SMILES: *[C@H]1CCN[C@H](C)C1]

In some embodiments, A is

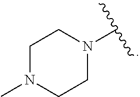 [SMILES: *N1CCN(C)CC1]

In some embodiments, A is

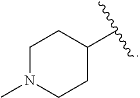 [SMILES: *C1CCN(C)CC1]

In some embodiments, A is

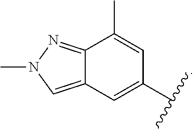 [SMILES: *c1cc(C)c2nn(C)cc2c1]

In some embodiments, A is

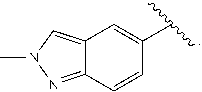 [SMILES: *c1ccc2nn(C)cc2c1]

In some embodiments, B is selected from

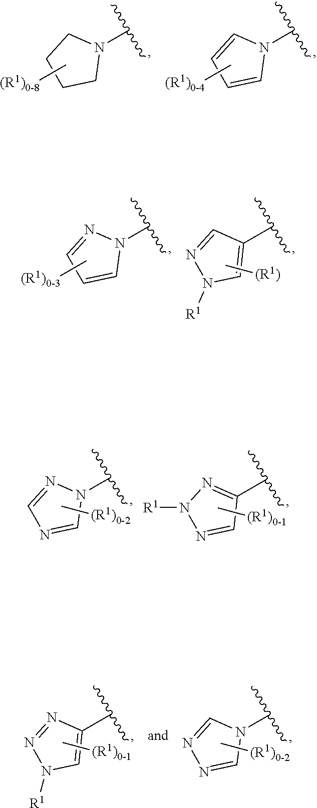

wherein R¹ is as defined herein. In some embodiments, B is selected from

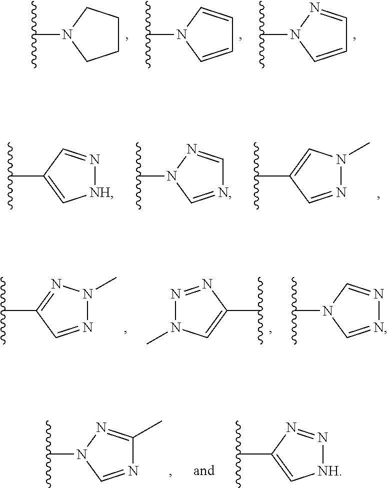

In some embodiments, B is

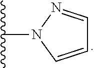 [SMILES: *n1cccn1]

In some embodiments, B is

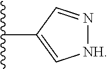 [SMILES: *c1cn[nH]c1]

In some embodiments, B is

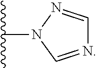 [SMILES: *n1cncn1]

In some embodiments, B is

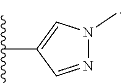 [SMILES: *c1cnn(C)c1]

In some embodiments, B is

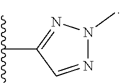 [SMILES: *c1cnn(C)n1]

In some embodiments, B is

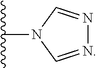 [SMILES: *n1cnnc1]

In some embodiments, B is

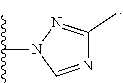 [SMILES: *n1cnc(C)n1]

In some embodiments, B is

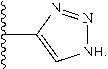 [SMILES: *c1c[nH]nn1]

In some embodiments, one of D, E, and F is N(R^{5f}), and the others of D, E, and F are each independently C(R^{5d}). In some embodiments, two of D, E, and F are each independently N(R^{5f}) or N, and the other of D, E, and F is C(R^{5d}). In some embodiments, three of D, E, and F are each independently N(R^{5f}) or N. In some embodiments, D is N(R^{5f}), and each of E and F are independently C(R^{5d}). In some embodiments, D is N(R^{5f}), E is C(R^{5d}), and F is N. In some embodiments, D is N(R^{5f}), and each of E and F are independently N. In some embodiments, one of D, E, and F is NH, and the others of D, E, and F are independently CH or N.

In some embodiments, one of D, E, and F is N(R^{5f}) (e.g., NH), and the others of D, E, and F are independently O or C(R^{5d})(R^{5e}) (e.g., C(O)). In some embodiments, D is N(R^{5f}) (e.g., CH), E, is C(R^{5d})(R^{5e}) (e.g., C(O)), and F is O. In some embodiments, D is O, E, is C(R^{5d})(R^{5e}) (e.g., C(O)), and F is N(R^{5f}) (e.g., NH). In some embodiments, D is NH, E, is C(O), and F is O. In some embodiments, D is O, E, is C(O), and F is NH.

In some embodiments, one of D, E, and F is S, and the others of D, E, and F, are each independently N or C(R^{5d}) (e.g. CH). In some embodiments, one of D, E, and F is S, and the others of D, E, and F, are each independently C(R^{5d}) (e.g., CH). In some embodiments, D is S, and E and F are each independently N or C(R^{5d}) (e.g., CH). In some embodiments, D is S, E is C(R^{5d}) (e.g., CH), and F is N. In some embodiments, D is S, and E and F are each independently C(R^{5d}) (e.g., CH). In some embodiments, D is S, E is CH, and F is N. In some embodiments, D is S, and E and F are each independently CH.

In some embodiments, one of D, E, and F is N(R^{5f}), and the others of D, E, and F are each independently C(R^{5d})(R^{5e}). In some embodiments, two of D, E, and F are each independently N(R^{5f}), and the other of D, E, and F is C(R^{5d})(R^{5e}). In some embodiments, D is N(R^{5f}), and each of E and F are independently C(R^{5d})(R^{5e}). In some embodiments, E is N(R^{5f}), and each of D and F are independently C(R^{5d})(R^{5e}). In some embodiments, F is N(R^{5f}), and each of D and E are independently C(R^{5d})(R^{5e}). In some embodiments, D is NH, E is C(O), and F is CH₂. In some embodiments, D is CH₂, E is NH, and F is C(O). In some embodiments, D is NH, E is CH, and F is C(O).

In some embodiments,

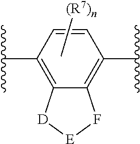

is selected from

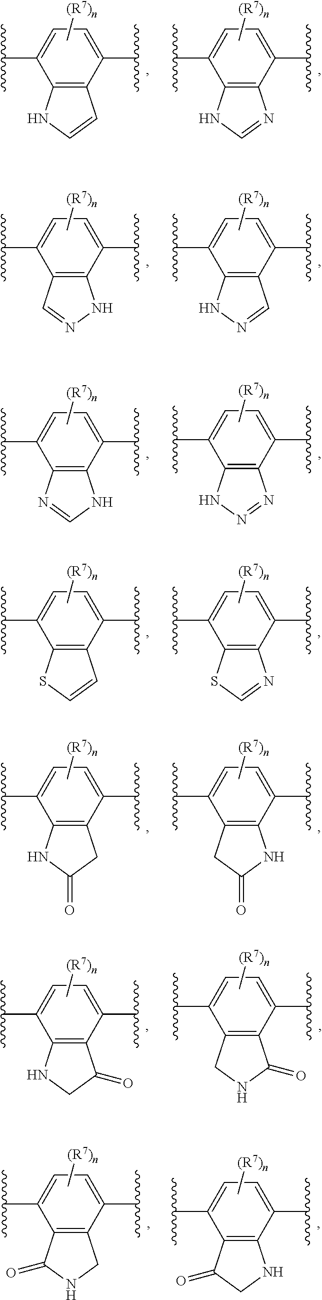

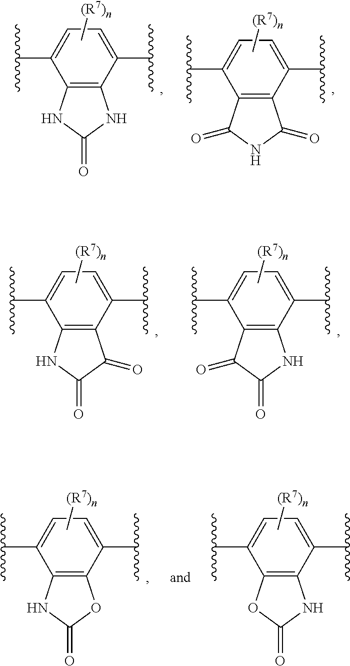

or a tautomer thereof, wherein R⁷ and n are as defined above.

In some embodiments,

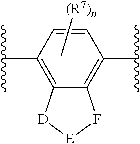

is selected from

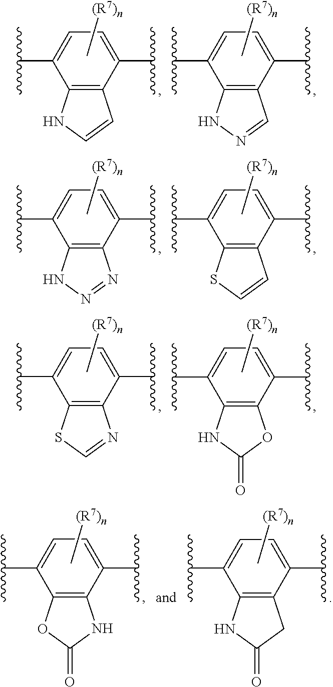

In some embodiments,

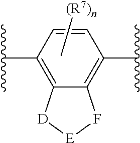

is selected from

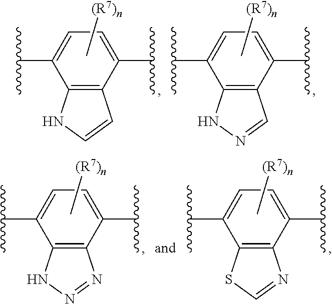

In some embodiments,

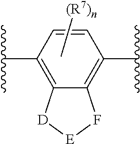

is

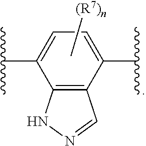

In some embodiments, L is absent. In some embodiments, L is oxygen. In some embodiments, L is nitrogen that is optionally substituted with R³. In some embodiments, L is nitrogen substituted with R³. In some embodiments, R³ is C₁-C₆ alkyl. In some embodiments, L is —N(CH₃)—. In some embodiments, L is —NH—.

In some embodiments, the compound of Formula (I) is a compound of Formula (I-f):

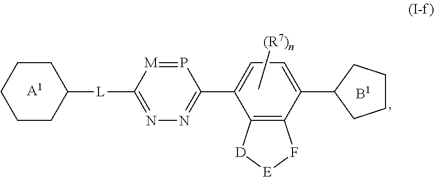

or a pharmaceutically acceptable salt, solvate, hydrate, tautomer, or stereoisomer thereof, wherein:

A¹ is 6-membered cycloalkyl, heterocyclyl, aryl, or heteroaryl, each of which is optionally substituted with one or more R¹;

B¹ is 5-membered cycloalkyl, heterocyclyl, aryl, or heteroaryl, each of which is optionally substituted with one or more R¹,

L is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴;

M and P are each independently C(R²) or N;

D, E, and F are each independently C, C(R^{5d}), C(R^{5d})(R^{5e}), N, N(R^{5f}), S, or O;

each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or

two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R′;

each R² is independently hydrogen, halo, cyano, C₁-C₆-alkyl, C₂-C₆-alkenyl, C₂-C₆-alkynyl, or —OR^{A};

each R³ is independently hydrogen, C₁-C₆-alkyl, or C₁-C₆-haloalkyl;

each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};

R^{5d} and R^{5e} are each independently hydrogen, halo, or C₁-C₆ alkyl; or

R^{5d} and R^{5e} are taken together to form an oxo group;

R^{5f} is hydrogen, halo, or C₁-C₆ alkyl;

each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹; or

two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹;

R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;

each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};

each of R^{B} and R^{C} is independently hydrogen, C₁-C₆ alkyl, C₁-C₆-heteroalkyl, cycloalkyl, heterocyclyl, —OR^{A}; or

R^{B} and R^{C} together with the atom to which they are attached form a 3-7-membered heterocyclyl ring optionally substituted with one or more R¹⁰;

each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;

each R¹⁰ is independently C₁-C₆-alkyl or halo;

each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};

m is 0, 1, or 2

n is 0, 1, 2, 3, or 4; and

x is 0, 1, or 2.

In some embodiments, A is selected from

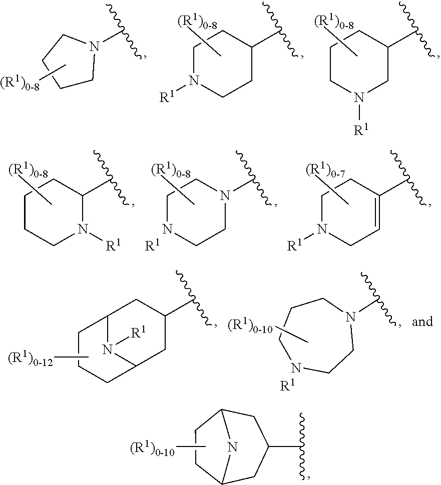

wherein R¹ is as defined herein.

In some embodiments, A is selected from

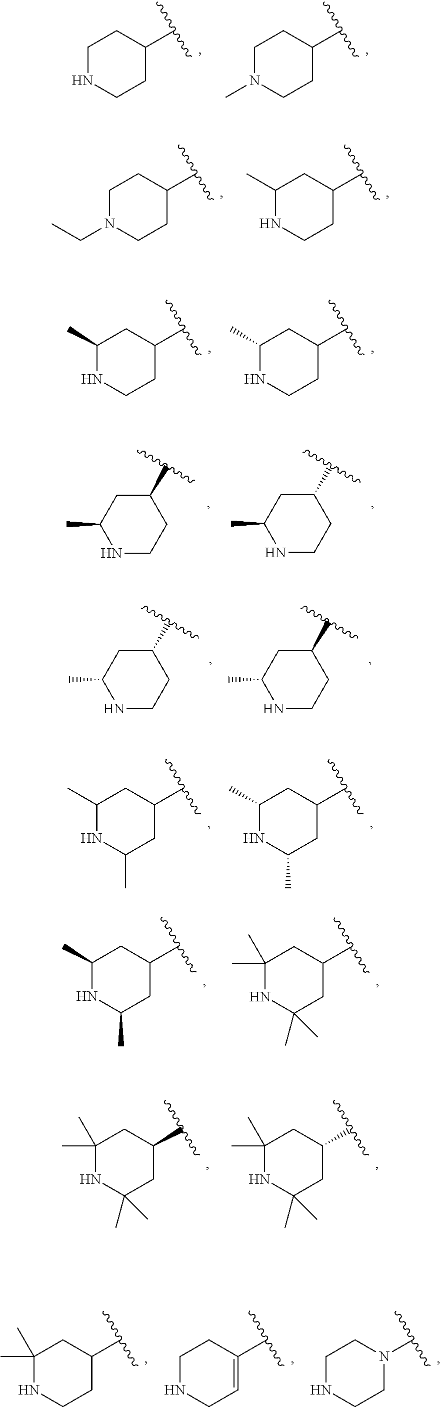

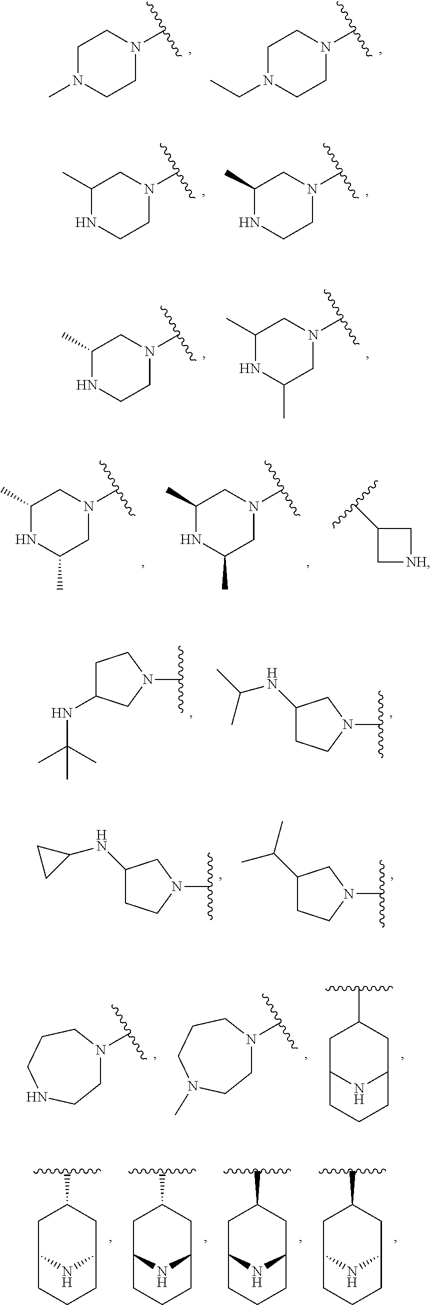

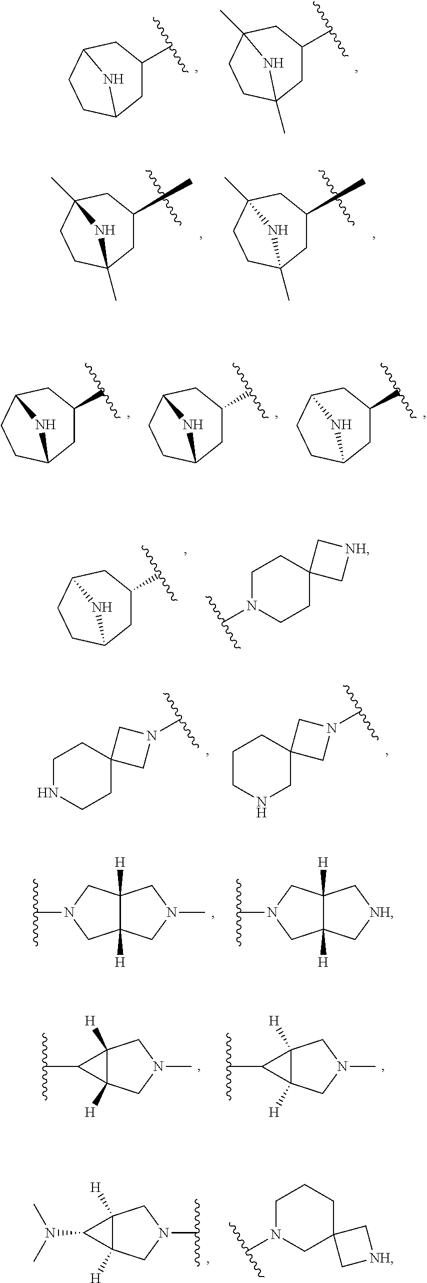

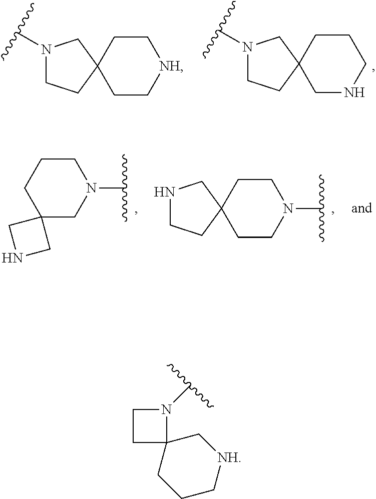

In some embodiments, A is selected from

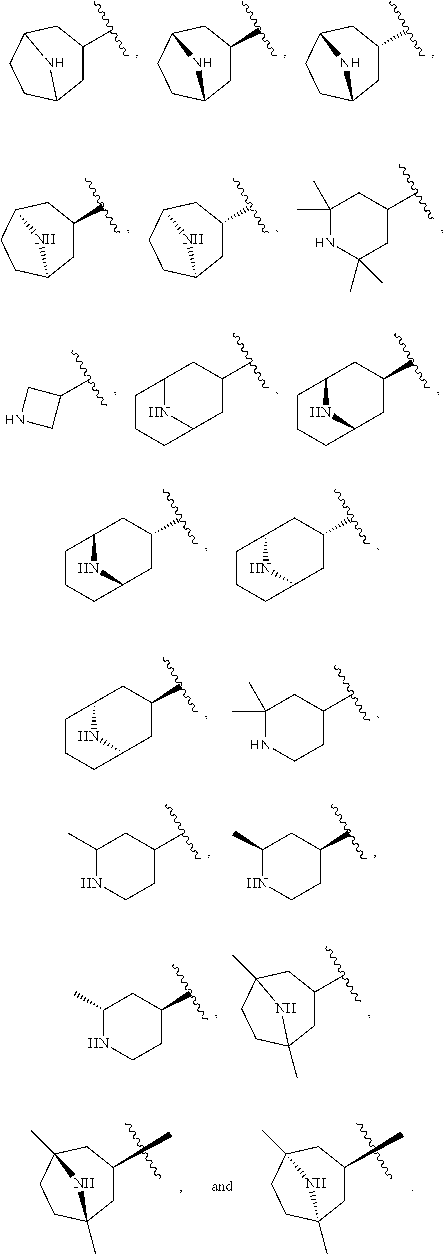

In some embodiments, A is selected from

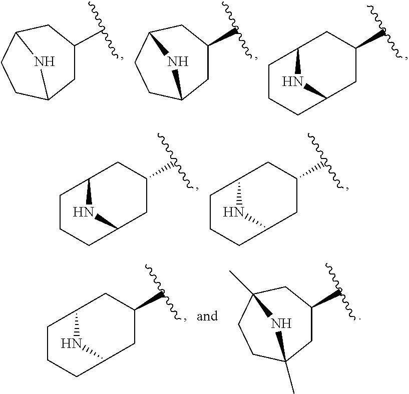

In some embodiments, A is

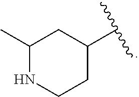 [SMILES: *C1CCNC(C)C1]

In some embodiments, A is

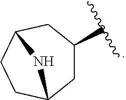 [SMILES: *[C@@H]1C[C@H]2CC[C@@H](C1)N2]

In some embodiments, A is

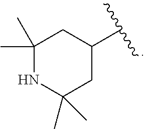 [SMILES: *C1CC(C)(C)NC(C)(C)C1]

In some embodiments, A is

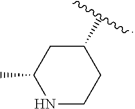 [SMILES: *[C@@H]1CCN[C@H](C)C1]

In some embodiments, A is

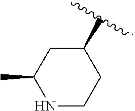 [SMILES: *[C@H]1CCN[C@@H](C)C1]

In some embodiments, A is

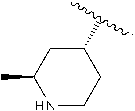 [SMILES: *[C@@H]1CCN[C@@H](C)C1]

In some embodiments, A is

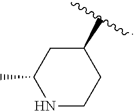 [SMILES: *[C@H]1CCN[C@H](C)C1]

In some embodiments, A is

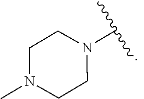 [SMILES: *N1CCN(C)CC1]

In some embodiments, A is

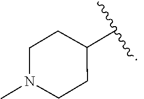 [SMILES: *C1CCN(C)CC1]

In some embodiments, A is

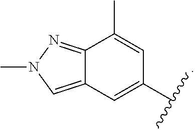 [SMILES: *c1cc(C)c2nn(C)cc2c1]

In some embodiments, A is

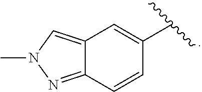 [SMILES: *c1ccc2nn(C)cc2c1]

In some embodiments, B is selected from

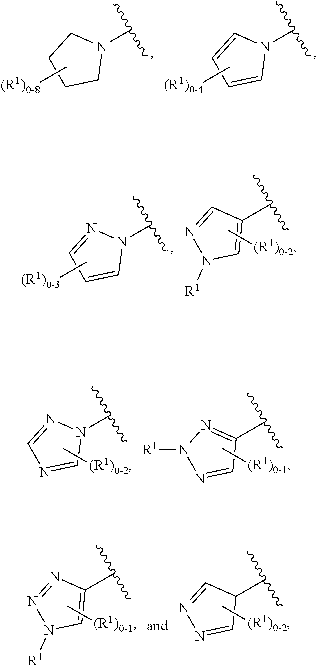

wherein R¹ is as defined herein. In some embodiments, B is selected from

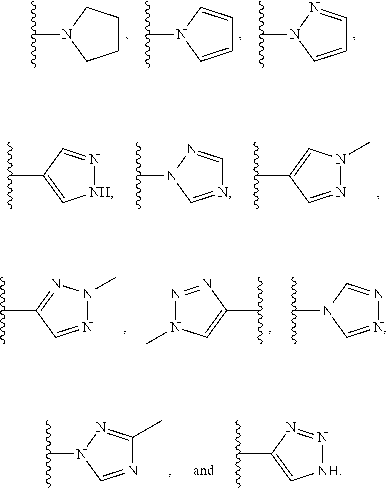

In some embodiments B is

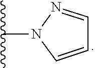 [SMILES: *n1cccn1]

In some embodiments, B is

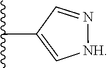 [SMILES: *c1cn[nH]c1]

In some embodiments, B is

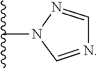 [SMILES: *n1cncn1]

In some embodiments, B is

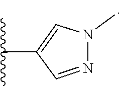 [SMILES: *c1cnn(C)c1]

In some embodiments, B is

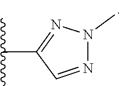 [SMILES: *c1cnn(C)n1]

In some embodiments, B is

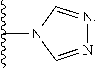 [SMILES: *n1cnnc1]

In some embodiments, B is

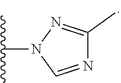 [SMILES: *n1cnc(C)n1]

In some embodiments, B is

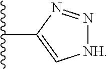 [SMILES: *c1c[nH]nn1]

In some embodiments, one of D, E, and F is N(R^{5f}), and the others of D, E, and F are each independently C(R^{5d}). In some embodiments, two of D, E, and F are each independently N(R^{5f}) or N, and the other of D, E, and F is C(R^{5d}). In some embodiments, three of D, E, and F are each independently N(R^{5f}) or N. In some embodiments, D is N(R^{5f}), and each of E and F are independently C(R^{5d}). In some embodiments, D is N(R^{5f}), E is C(R^{5d}), and F is N. In some embodiments, D is N(R^{5f}), and each of E and F are independently N. In some embodiments, one of D, E, and F is NH, and the others of D, E, and F are independently CH or N.

In some embodiments, one of D, E, and F is N(R^{5f}) (e.g., NH), and the others of D, E, and F are independently O or C(R^{5d})(R^{5e}) (e.g., C(O)). In some embodiments, D is N(R^{5f}) (e.g., CH), E, is C(R^{5d})(R^{5e}) (e.g., C(O)), and F is O. In some embodiments, D is O, E, is C(R^{5d})(R^{5e}) (e.g., C(O)), and F is N(R^{5f}) (e.g., NH). In some embodiments, D is NH, E, is C(O), and F is O. In some embodiments, D is O, E, is C(O), and F is NH.

In some embodiments, one of D, E, and F is S, and the others of D, E, and F, are each independently N or C(R^{5d}) (e.g. CH). In some embodiments, one of D, E, and F is S, and the others of D, E, and F, are each independently C(R^{5d}) (e.g., CH). In some embodiments, D is S, and E and F are each independently N or C(R^{5d}) (e.g., CH). In some embodiments, D is S, E is C(R^{5d}) (e.g., CH), and F is N. In some embodiments, D is S, and E and F are each independently C(R^{5d}) (e.g., CH). In some embodiments, D is S, E is CH, and F is N. In some embodiments, D is S, and E and F are each independently CH.

In some embodiments, one of D, E, and F is N(R^{5S}), and the others of D, E, and F are each independently C(R^{5d})(R^{5e}). In some embodiments, two of D, E, and F are each independently N(R^{5S}), and the other of D, E, and F is C(R^{5d})(R^{5e}). In some embodiments, D is N(R^{5S}), and each of E and F are independently C(R^{5d})(R^{5e}). In some embodiments, E is N(R^{5f}), and each of D and F are independently C(R^{5d})(R^{5e}). In some embodiments, F is N(R^{5S}), and each of D and E are independently C(R^{5d})(R^{5e}). In some embodiments, D is NH, E is C(O), and F is CH₂. In some embodiments, D is CH₂, E is NH, and F is C(O). In some embodiments, D is NH, E is CH, and F is C(O).

In some embodiments,

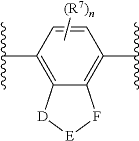

is selected from

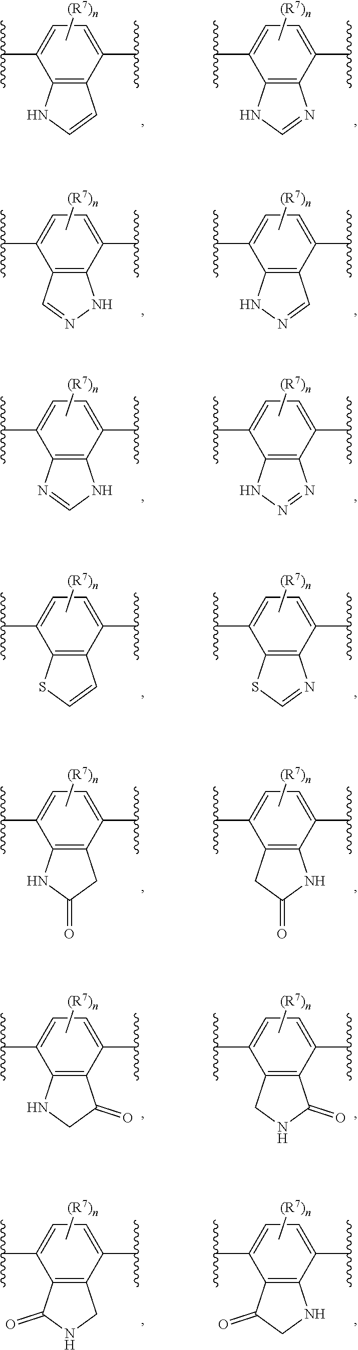

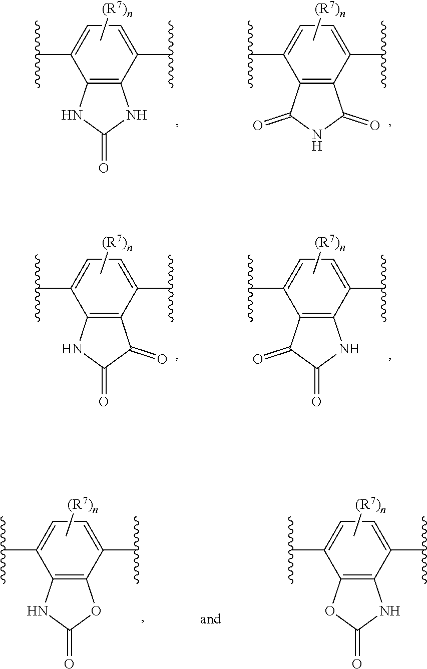

or a tautomer thereof, wherein R⁷ and n are as defined above.

In some embodiments,

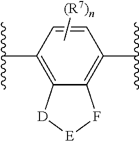

is selected from

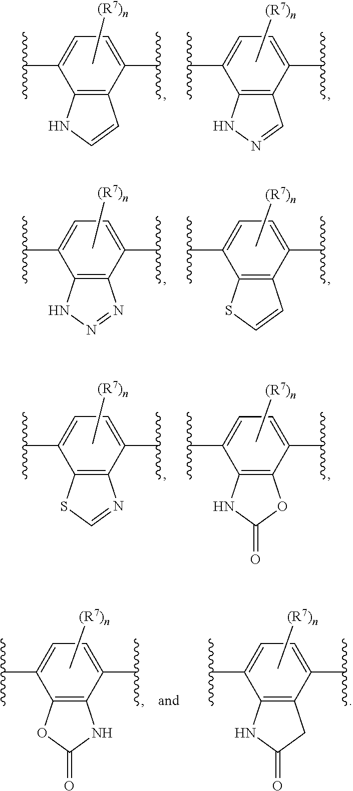

In some embodiments,

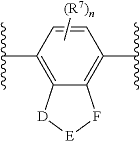

is selected from

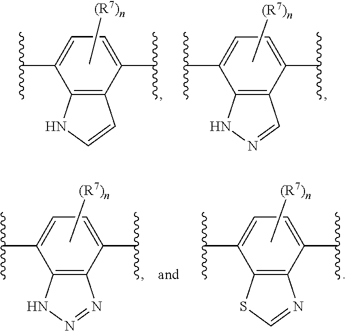

In some embodiments,

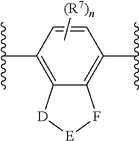

is

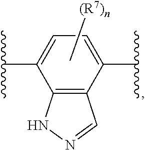

In some embodiments, L is absent. In some embodiments, L is oxygen. In some embodiments, L is nitrogen that is optionally substituted with R³. In some embodiments, L is nitrogen substituted with R³. In some embodiments, R³ is C₁-C₆ alkyl. In some embodiments, L is —N(CH₃)—. In some embodiments, L is —NH—.

In some embodiments, the compound of Formula (I) is a compound of Formula (I-g):

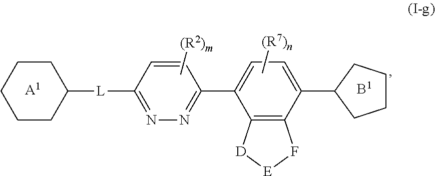

or a pharmaceutically acceptable salt, solvate, hydrate, tautomer, or stereoisomer thereof, wherein:

A¹ is 6-membered cycloalkyl, heterocyclyl, aryl, or heteroaryl, each of which is optionally substituted with one or more R¹;

B¹ is 5-membered cycloalkyl, heterocyclyl, aryl, or heteroaryl, each of which is optionally substituted with one or more R¹,

L is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴;

D, E, and F are each independently C, C(R^{5d}), C(R^{5d})(R^{5e}), N, N(R^{5f}), S, or O;

each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or

two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R′;

each R² is independently hydrogen, halo, cyano, C₁-C₆-alkyl, C₂-C₆-alkenyl, C₂-C₆-alkynyl, or —OR^{A};

each R³ is independently hydrogen, C₁-C₆-alkyl, or C₁-C₆-haloalkyl;

each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};

R^{5d} and R^{5e} are each independently hydrogen, halo, or C₁-C₆ alkyl; or

R^{5d} and R^{5e} are taken together to form an oxo group;

R^{5f} is hydrogen, halo, or C₁-C₆ alkyl;

each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹ or

two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹;

R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;

each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};

each of R^{B} and R^{C} is independently hydrogen, C₁-C₆ alkyl, C₁-C₆-heteroalkyl, cycloalkyl, heterocyclyl, —OR^{A}; or

R^{B} and R^{C} together with the atom to which they are attached form a 3-7-membered heterocyclyl ring optionally substituted with one or more R¹⁰;

each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;

each R¹⁰ is independently C₁-C₆-alkyl or halo;

each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};

m is 0, 1, or 2

n is 0, 1, 2, 3, or 4; and

x is 0, 1, or 2.

In some embodiments, A¹ is 6-membered heterocyclyl optionally substituted with one or more R¹. In some embodiments, A¹ is 6-membered nitrogen-containing heterocyclyl. In some embodiments, A¹ is optionally substituted piperidinyl. In some embodiments, A¹ is

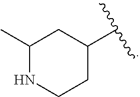 [SMILES: *C1CCNC(C)C1]

In some embodiments, A¹ is

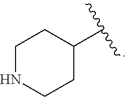 [SMILES: *C1CCNCC1]

In some embodiments, A¹ is

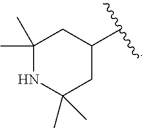 [SMILES: *C1CC(C)(C)NC(C)(C)C1]

In some embodiments, A¹ is

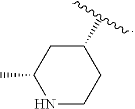 [SMILES: *[C@@H]1CCN[C@H](C)C1]

In some embodiments, A¹ is

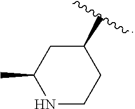 [SMILES: *[C@H]1CCN[C@@H](C)C1]

In some embodiments, A1 is

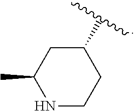 [SMILES: *[C@@H]1CCN[C@@H](C)C1]

In some embodiments, A1 is

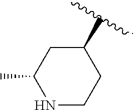 [SMILES: *[C@H]1CCN[C@H](C)C1]

In some embodiments, A1 is

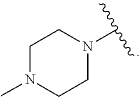 [SMILES: *N1CCN(C)CC1]

In some embodiments, A1 is

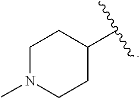 [SMILES: *C1CCN(C)CC1]

In some embodiments, L is nitrogen substituted with R³. In some embodiments, L is —N(CH₃)—. In some embodiments, L is —NH—. In some embodiments, B¹ is 5-membered heteroaryl optionally substituted with one or more R¹. In some embodiments, B¹ is 5-membered nitrogen-containing heteroaryl. In some embodiments, B¹ is optionally substituted pyrazolyl. In some embodiments, B1 is

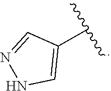 [SMILES: *c1cn[nH]c1]

In some embodiments, B1 is

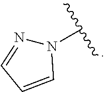 [SMILES: *n1cccn1]

In some embodiments, B1 is

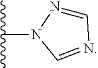 [SMILES: *n1cncn1]

In some embodiments, B1 is

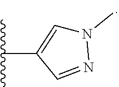 [SMILES: *c1cnn(C)c1]

In some embodiments, B1 is

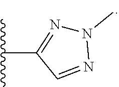 [SMILES: *c1cnn(C)n1]

In some embodiments, B1 is

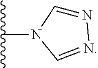 [SMILES: *n1cnnc1]

In some embodiments, B1 is

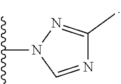 [SMILES: *n1cnc(C)n1]

In some embodiments, B1 is

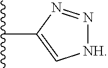 [SMILES: *c1c[nH]nn1]

In some embodiments, B1 is

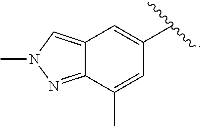 [SMILES: *c1cc(C)c2nn(C)cc2c1]

In some embodiments, B1 is

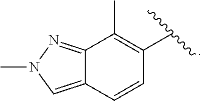 [SMILES: *c1ccc2cn(C)nc2c1C]

In some embodiments, B1 is

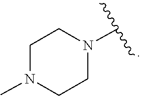 [SMILES: *N1CCN(C)CC1]

In some embodiments, B1 is

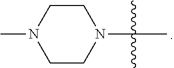 [SMILES: *N1CCN(C)CC1]

In some embodiments, B1 is

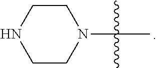 [SMILES: *N1CCNCC1]

In some embodiments,

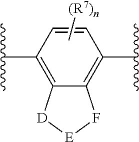

is selected from

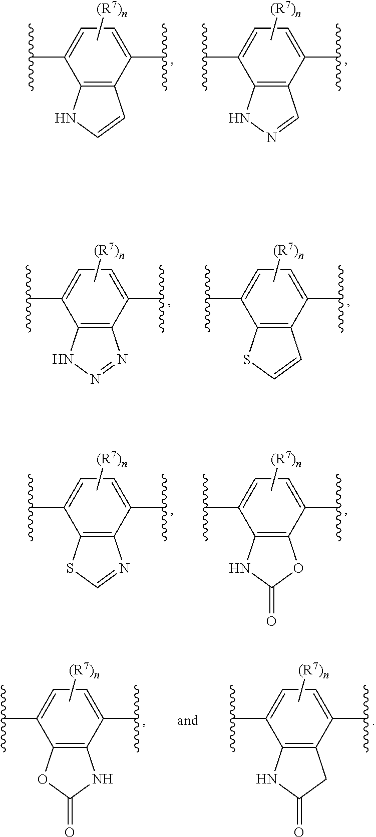

In some embodiments,

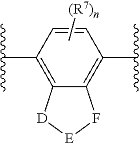

is selected from

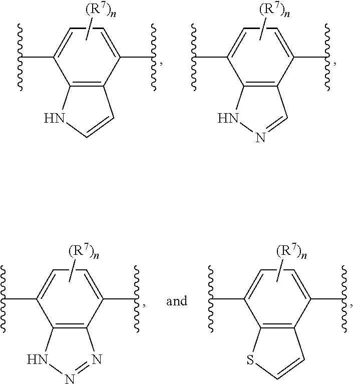

In some embodiments,

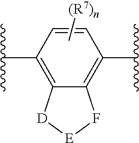

is

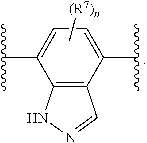

In some embodiments, the compound of Formula (I) is a compound of Formula (I-h):

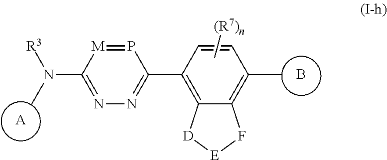

or a pharmaceutically acceptable salt, solvate, hydrate, tautomer, or stereoisomer thereof, wherein:

A and B are each independently cycloalkyl, heterocyclyl, aryl, or heteroaryl, each of which is optionally substituted with one or more R¹;

M and P are each independently C(R²) or N;

D, E, and F are each independently C(R^{5d}), C(R^{5d})(R^{5e}), N, N(R^{5f}), S, or O, wherein the bonds between the atoms in the ring comprising D, E, and F may be a single bonds or double bonds as valency permits;

each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or

two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R′;

each R² is independently hydrogen, halo, cyano, C₁-C₆-alkyl, C₂-C₆-alkenyl, C₂-C₆-alkynyl, or —OR^{A};

each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R¹²;

R^{5d} and R^{5c} are each independently hydrogen, halo, or C₁-C₆ alkyl; or

R^{5d} and R^{5c} are taken together to form an oxo group;

R^{5f} is hydrogen, halo, or C₁-C₆ alkyl;

each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹ or

two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹;

R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;

each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};

each of R^{B} and R^{C} is independently hydrogen, C₁-C₆ alkyl, C₁-C₆-heteroalkyl, cycloalkyl, heterocyclyl, —OR^{A}; or

R^{B} and R^{C} together with the atom to which they are attached form a 3-7-membered heterocyclyl ring optionally substituted with one or more R¹⁰;

each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;

each R¹⁰ is independently C₁-C₆-alkyl or halo;

each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

m is 0, 1, or 2

n is 0, 1, 2, 3, or 4; and

x is 0, 1, or 2.

In some embodiments, the compound of Formula (I) is a compound of Formula (I-i):

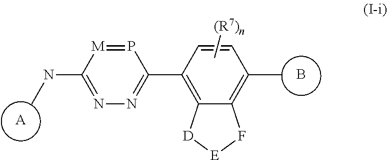

or a pharmaceutically acceptable salt, solvate, hydrate, tautomer, or stereoisomer thereof, wherein:

A and B are each independently cycloalkyl, heterocyclyl, aryl, or heteroaryl, each of which is optionally substituted with one or more R¹;

M and P are each independently C(R²) or N;

D, E, and F are each independently C(R^{5d}), C(R^{5d})(R^{5e}), N, N(R^{5f}), S, or O, wherein the bonds between the atoms in the ring comprising D, E, and F may be a single bonds or double bonds as valency permits;

each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or

two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R′;

each R² is independently hydrogen, halo, cyano, C₁-C₆-alkyl, C₂-C₆-alkenyl, C₂-C₆-alkynyl, or —OR^{A};

R^{5d} and R^{5c} are each independently hydrogen, halo, or C₁-C₆ alkyl; or

R^{5d} and R^{5c} are taken together to form an oxo group;

R^{5f} is hydrogen, halo, or C₁-C₆ alkyl;

each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹ or

two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹;

R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;

each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};

each of R^{B} and R^{C} is independently hydrogen, C₁-C₆ alkyl, C₁-C₆-heteroalkyl, cycloalkyl, heterocyclyl, —OR^{A}; or

R^{B} and R^{C} together with the atom to which they are attached form a 3-7-membered heterocyclyl ring optionally substituted with one or more R¹⁰;

each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;

each R¹⁰ is independently C₁-C₆-alkyl or halo;

each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

m is 0, 1, or 2

n is 0, 1, 2, 3, or 4; and

x is 0, 1, or 2.

In some embodiments, the compound of Formula (I) is a compound of Formula (I-j):

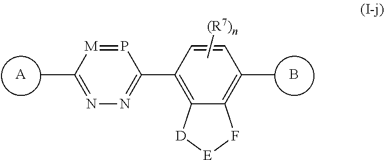

or a pharmaceutically acceptable salt, solvate, hydrate, tautomer, or stereoisomer thereof, wherein:

A and B are each independently cycloalkyl, heterocyclyl, aryl, or heteroaryl, each of which is optionally substituted with one or more R¹;

M and P are each independently C(R²) or N;

D, E, and F are each independently C(R^{5d}), C(R^{5d})(R^{5e}), N, N(R^{5f}), S, or O, wherein the bonds between the atoms in the ring comprising D, E, and F may be a single bonds or double bonds as valency permits;

each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or

two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R′;

each R² is independently hydrogen, halo, cyano, C₁-C₆-alkyl, C₂-C₆-alkenyl, C₂-C₆-alkynyl, or —OR^{A};

R^{5d} and R^{5c} are each independently hydrogen, halo, or C₁-C₆ alkyl; or

R^{5d} and R^{5c} are taken together to form an oxo group;

R^{5f} is hydrogen, halo, or C₁-C₆ alkyl;

each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹ or

two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹;

R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;

each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};

each of R^{B} and R^{C} is independently hydrogen, C₁-C₆ alkyl, C₁-C₆-heteroalkyl, cycloalkyl, heterocyclyl, —OR^{A}; or

R^{B} and R^{C} together with the atom to which they are attached form a 3-7-membered heterocyclyl ring optionally substituted with one or more R¹⁰;

each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;

each R¹⁰ is independently C₁-C₆-alkyl or halo;

each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

m is 0, 1, or 2

n is 0, 1, 2, 3, or 4; and

x is 0, 1, or 2.

In some embodiments, the compound of Formula (I) is a compound of Formula (I-k):

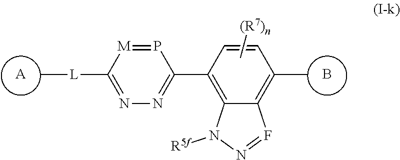

or a pharmaceutically acceptable salt, solvate, hydrate, tautomer, or stereoisomer thereof, wherein:

A and B are each independently cycloalkyl, heterocyclyl, aryl, or heteroaryl, each of which is optionally substituted with one or more R¹;

L is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴;

M and P are each independently C(R²) or N;

F is C(R^{5d}) or N;

each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or

two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R′;

each R² is independently hydrogen, halo, cyano, C₁-C₆-alkyl, C₂-C₆-alkenyl, C₂-C₆-alkynyl, or —OR^{A};

each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R²;

each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};

R^{5d} is hydrogen, halo, or C₁-C₆ alkyl;

R^{5f} is hydrogen, halo, or C₁-C₆ alkyl;

each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹ or

two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹;

R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;

each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};

each of R^{B} and R^{C} is independently hydrogen, C₁-C₆ alkyl, C₁-C₆-heteroalkyl, cycloalkyl, heterocyclyl, —OR^{A}; or

R^{B} and R^{C} together with the atom to which they are attached form a 3-7-membered heterocyclyl ring optionally substituted with one or more R¹⁰;

each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;

each R¹⁰ is independently C₁-C₆-alkyl or halo;

each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

m is 0, 1, or 2

n is 0, 1, 2, 3, or 4; and

x is 0, 1, or 2.

In some embodiments, the compound of Formula (I) is a compound of Formula (I-1):

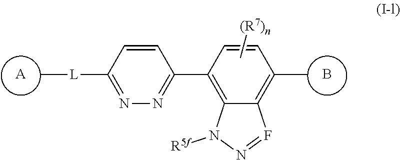

or a pharmaceutically acceptable salt, solvate, hydrate, tautomer, or stereoisomer thereof, wherein:

A and B are each independently cycloalkyl, heterocyclyl, aryl, or heteroaryl, each of which is optionally substituted with one or more R¹;

L is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴;

F is C(R^{5d}) or N;

each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or

two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R′;

each R² is independently hydrogen, halo, cyano, C₁-C₆-alkyl, C₂-C₆-alkenyl, C₂-C₆-alkynyl, or —OR^{A};

each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R²;

each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};

R^{5d} is hydrogen, halo, or C₁-C₆ alkyl;

R^{5f} is hydrogen, halo, or C₁-C₆ alkyl;

each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹ or

two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹;

R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;

each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};

each of R^{B} and R^{C} is independently hydrogen, C₁-C₆ alkyl, C₁-C₆-heteroalkyl, cycloalkyl, heterocyclyl, —OR^{A}; or

R^{B} and R^{C} together with the atom to which they are attached form a 3-7-membered heterocyclyl ring optionally substituted with one or more R¹⁰;

each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;

each R¹⁰ is independently C₁-C₆-alkyl or halo;

each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

m is 0, 1, or 2

n is 0, 1, 2, 3, or 4; and

x is 0, 1, or 2.

In some embodiments, the compound of Formula (I) is a compound of Formula (I-m):

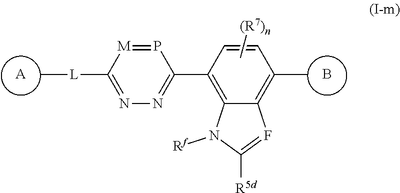

or a pharmaceutically acceptable salt, solvate, hydrate, tautomer, or stereoisomer thereof, wherein:

A and B are each independently cycloalkyl, heterocyclyl, aryl, or heteroaryl, each of which is optionally substituted with one or more R¹;

L is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴;

M and P are each independently C(R²) or N;

F is C(R^{5d}) or N;

each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or

two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R′;

each R² is independently hydrogen, halo, cyano, C₁-C₆-alkyl, C₂-C₆-alkenyl, C₂-C₆-alkynyl, or —OR^{A};

each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R²;

each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};

R^{5d} is hydrogen, halo, or C₁-C₆ alkyl;

R^{5f} is hydrogen, halo, or C₁-C₆ alkyl;

each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹ or

two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹;

R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;

each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};

each of R^{B} and R^{C} is independently hydrogen, C₁-C₆ alkyl, C₁-C₆-heteroalkyl, cycloalkyl, heterocyclyl, —OR^{A}; or

R^{B} and R^{C} together with the atom to which they are attached form a 3-7-membered heterocyclyl ring optionally substituted with one or more R¹⁰;

each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;

each R¹⁰ is independently C₁-C₆-alkyl or halo;

each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

m is 0, 1, or 2

n is 0, 1, 2, 3, or 4; and

x is 0, 1, or 2.

In some embodiments, the compound of Formula (I) is a compound of Formula (I-n):

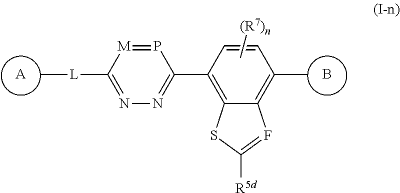

or a pharmaceutically acceptable salt, solvate, hydrate, tautomer, or stereoisomer thereof, wherein:

A and B are each independently cycloalkyl, heterocyclyl, aryl, or heteroaryl, each of which is optionally substituted with one or more R¹;

L is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴;

M and P are each independently C(R²) or N;

F is C(R^{5d}) or N;

each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or

two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R′;

each R² is independently hydrogen, halo, cyano, C₁-C₆-alkyl, C₂-C₆-alkenyl, C₂-C₆-alkynyl, or —OR^{A};

each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R²;

each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};

R^{5d} is hydrogen, halo, or C₁-C₆ alkyl;

each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹ or

two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹;

R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;

each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};

each of R^{B} and R^{C} is independently hydrogen, C₁-C₆ alkyl, C₁-C₆-heteroalkyl, cycloalkyl, heterocyclyl, —OR^{A}; or

R^{B} and R^{C} together with the atom to which they are attached form a 3-7-membered heterocyclyl ring optionally substituted with one or more R¹⁰;

each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;

each R¹⁰ is independently C₁-C₆-alkyl or halo;

each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};

m is 0, 1, or 2

n is 0, 1, 2, 3, or 4; and

x is 0, 1, or 2.

In some embodiments, the compound of Formula (I) is selected from a compound in Table 1, or a pharmaceutically acceptable salt thereof.

TABLE 1
Exemplary compounds

| Compound No. | Structure |
|---|---|
| 100 | 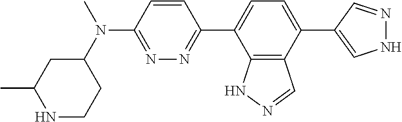 [SMILES: CC1CC(N(C)c2ccc(-c3ccc(-c4cn[nH]c4)c4cn[nH]c34)nn2)CCN1] |
| 101 | 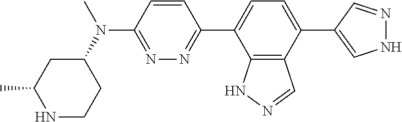 [SMILES: C[C@@H]1C[C@H](N(C)c2ccc(-c3ccc(-c4cn[nH]c4)c4cn[nH]c34)nn2)CCN1] |
| 102 | 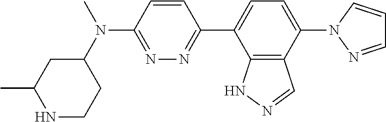 [SMILES: CC1CC(N(C)c2ccc(-c3ccc(-n4cccn4)c4cn[nH]c34)nn2)CCN1] |
| 103 | 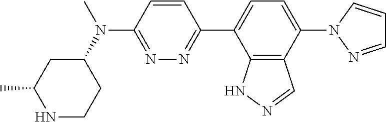 [SMILES: C[C@@H]1C[C@H](N(C)c2ccc(-c3ccc(-n4cccn4)c4cn[nH]c34)nn2)CCN1] |
| 104 | 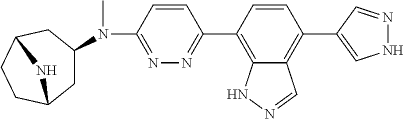 [SMILES: CN(c1ccc(-c2ccc(-c3cn[nH]c3)c3cn[nH]c23)nn1)[C@@H]1C[C@H]2CC[C@@H](C1)N2] |
| 105 | 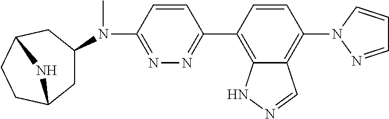 [SMILES: CN(c1ccc(-c2ccc(-n3cccn3)c3cn[nH]c23)nn1)[C@@H]1C[C@H]2CC[C@@H](C1)N2] |
| 106 | 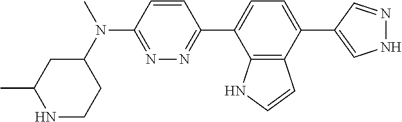 [SMILES: CC1CC(N(C)c2ccc(-c3ccc(-c4cn[nH]c4)c4cc[nH]c34)nn2)CCN1] |
| 107 | 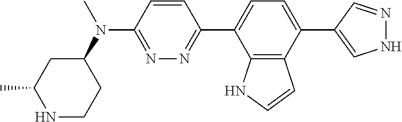 [SMILES: C[C@@H]1C[C@@H](N(C)c2ccc(-c3ccc(-c4cn[nH]c4)c4cc[nH]c34)nn2)CCN1] |
| 108 | 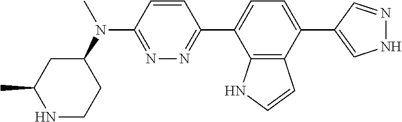 [SMILES: C[C@H]1C[C@@H](N(C)c2ccc(-c3ccc(-c4cn[nH]c4)c4cc[nH]c34)nn2)CCN1] |
| 109 | 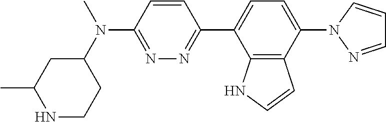 [SMILES: CC1CC(N(C)c2ccc(-c3ccc(-n4cccn4)c4cc[nH]c34)nn2)CCN1] |
| 110 | 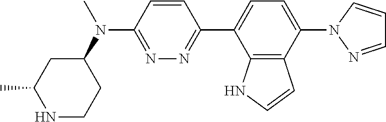 [SMILES: C[C@@H]1C[C@@H](N(C)c2ccc(-c3ccc(-n4cccn4)c4cc[nH]c34)nn2)CCN1] |
| 111 | 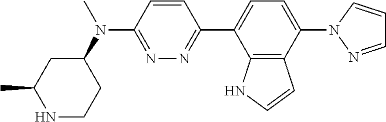 [SMILES: C[C@H]1C[C@@H](N(C)c2ccc(-c3ccc(-n4cccn4)c4cc[nH]c34)nn2)CCN1] |
| 112 | 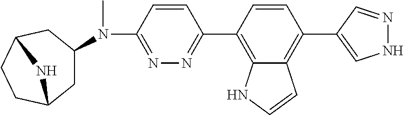 [SMILES: CN(c1ccc(-c2ccc(-c3cn[nH]c3)c3cc[nH]c23)nn1)[C@@H]1C[C@H]2CC[C@@H](C1)N2] |
| 113 | 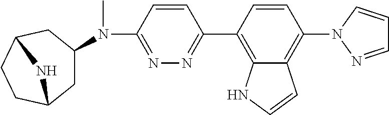 [SMILES: CN(c1ccc(-c2ccc(-n3cccn3)c3cc[nH]c23)nn1)[C@@H]1C[C@H]2CC[C@@H](C1)N2] |
| 114 | 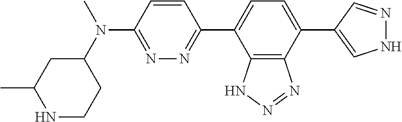 [SMILES: CC1CC(N(C)c2ccc(-c3ccc(-c4cn[nH]c4)c4nn[nH]c34)nn2)CCN1] |
| 115 | 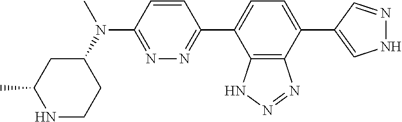 [SMILES: C[C@@H]1C[C@H](N(C)c2ccc(-c3ccc(-c4cn[nH]c4)c4nn[nH]c34)nn2)CCN1] |
| 116 | 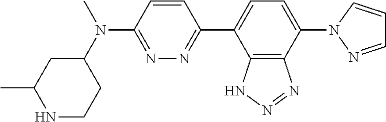 [SMILES: CC1CC(N(C)c2ccc(-c3ccc(-n4cccn4)c4nn[nH]c34)nn2)CCN1] |
| 117 | 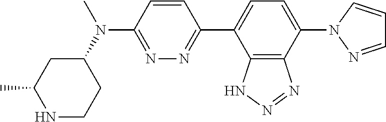 [SMILES: C[C@@H]1C[C@H](N(C)c2ccc(-c3ccc(-n4cccn4)c4nn[nH]c34)nn2)CCN1] |
| 118 | 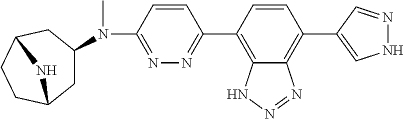 [SMILES: CN(c1ccc(-c2ccc(-c3cn[nH]c3)c3nn[nH]c23)nn1)[C@@H]1C[C@H]2CC[C@@H](C1)N2] |
| 119 | 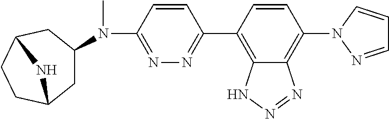 [SMILES: CN(c1ccc(-c2ccc(-n3cccn3)c3nn[nH]c23)nn1)[C@@H]1C[C@H]2CC[C@@H](C1)N2] |
| 120 | 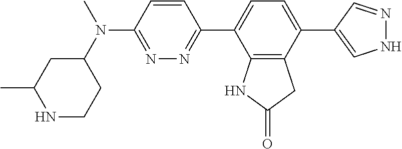 [SMILES: CC1CC(N(C)c2ccc(-c3ccc(-c4cn[nH]c4)c4c3NC(=O)C4)nn2)CCN1] |
| 121 | 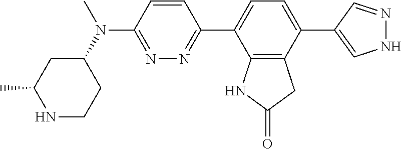 [SMILES: C[C@@H]1C[C@H](N(C)c2ccc(-c3ccc(-c4cn[nH]c4)c4c3NC(=O)C4)nn2)CCN1] |
| 122 | 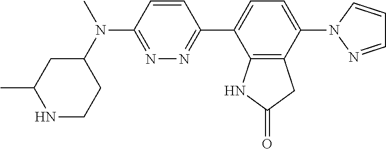 [SMILES: CC1CC(N(C)c2ccc(-c3ccc(-n4cccn4)c4c3NC(=O)C4)nn2)CCN1] |
| 123 | 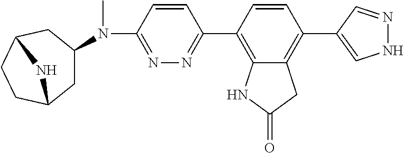 [SMILES: CN(c1ccc(-c2ccc(-c3cn[nH]c3)c3c2NC(=O)C3)nn1)[C@@H]1C[C@H]2CC[C@@H](C1)N2] |
| 124 | 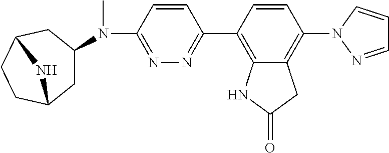 [SMILES: CN(c1ccc(-c2ccc(-n3cccn3)c3c2NC(=O)C3)nn1)[C@@H]1C[C@H]2CC[C@@H](C1)N2] |
| 131 | 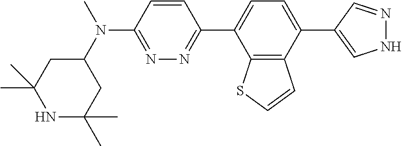 [SMILES: CN(c1ccc(-c2ccc(-c3cn[nH]c3)c3ccsc23)nn1)C1CC(C)(C)NC(C)(C)C1] |
| 132 | 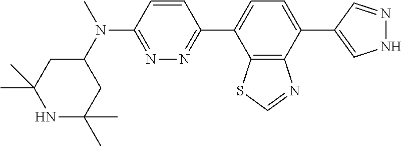 [SMILES: CN(c1ccc(-c2ccc(-c3cn[nH]c3)c3ncsc23)nn1)C1CC(C)(C)NC(C)(C)C1] |
| 137 | 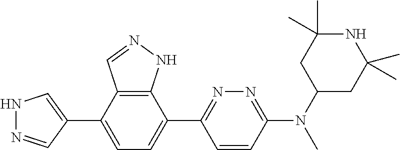 [SMILES: CN(c1ccc(-c2ccc(-c3cn[nH]c3)c3cn[nH]c23)nn1)C1CC(C)(C)NC(C)(C)C1] |
| 138 | 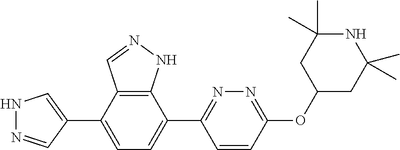 [SMILES: CC1(C)CC(Oc2ccc(-c3ccc(-c4cn[nH]c4)c4cn[nH]c34)nn2)CC(C)(C)N1] |
| 139 | 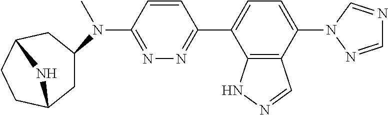 [SMILES: CN(c1ccc(-c2ccc(-n3cncn3)c3cn[nH]c23)nn1)[C@@H]1C[C@H]2CC[C@@H](C1)N2] |
| 140 | 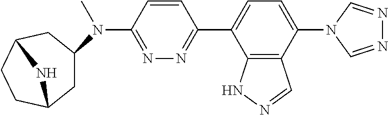 [SMILES: CN(c1ccc(-c2ccc(-n3cnnc3)c3cn[nH]c23)nn1)[C@@H]1C[C@H]2CC[C@@H](C1)N2] |
| 141 | 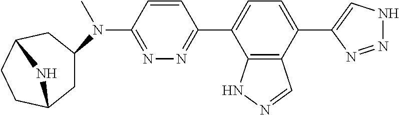 [SMILES: CN(c1ccc(-c2ccc(-c3c[nH]nn3)c3cn[nH]c23)nn1)[C@@H]1C[C@H]2CC[C@@H](C1)N2] |
| 142 | 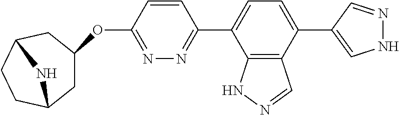 [SMILES: c1n[nH]cc1-c1ccc(-c2ccc(O[C@@H]3C[C@H]4CC[C@@H](C3)N4)nn2)c2[nH]ncc12] |
| 143 | 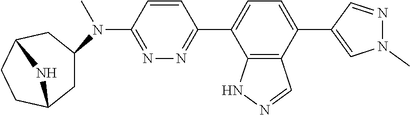 [SMILES: CN(c1ccc(-c2ccc(-c3cnn(C)c3)c3cn[nH]c23)nn1)[C@@H]1C[C@H]2CC[C@@H](C1)N2] |
| 144 | 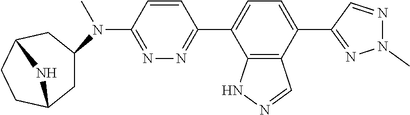 [SMILES: CN(c1ccc(-c2ccc(-c3cnn(C)n3)c3cn[nH]c23)nn1)[C@@H]1C[C@H]2CC[C@@H](C1)N2] |
| 145 | 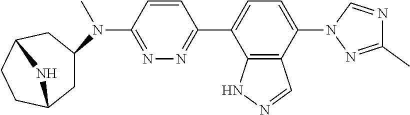 [SMILES: Cc1ncn(-c2ccc(-c3ccc(N(C)[C@@H]4C[C@H]5CC[C@@H](C4)N5)nn3)c3[nH]ncc23)n1] |
| 146 | 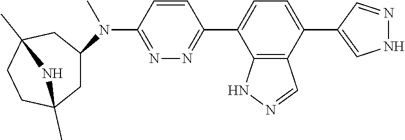 [SMILES: CN(c1ccc(-c2ccc(-c3cn[nH]c3)c3cn[nH]c23)nn1)[C@H]1C[C@]2(C)CC[C@](C)(C1)N2] |
| 147 | 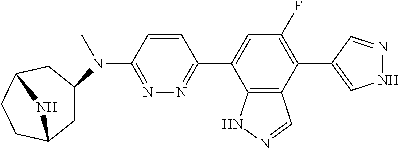 [SMILES: CN(c1ccc(-c2cc(F)c(-c3cn[nH]c3)c3cn[nH]c23)nn1)[C@@H]1C[C@H]2CC[C@@H](C1)N2] |
| 148 | 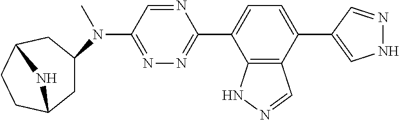 [SMILES: CN(c1cnc(-c2ccc(-c3cn[nH]c3)c3cn[nH]c23)nn1)[C@@H]1C[C@H]2CC[C@@H](C1)N2] |
| 149 | 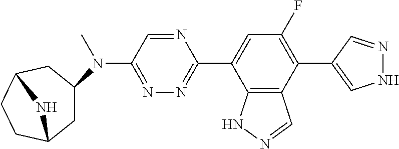 [SMILES: CN(c1cnc(-c2cc(F)c(-c3cn[nH]c3)c3cn[nH]c23)nn1)[C@@H]1C[C@H]2CC[C@@H](C1)N2] |
| 150 | 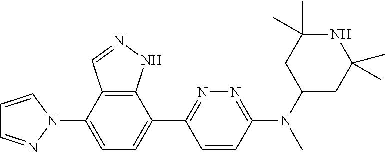 [SMILES: CN(c1ccc(-c2ccc(-n3cccn3)c3cn[nH]c23)nn1)C1CC(C)(C)NC(C)(C)C1] |
| 151 | 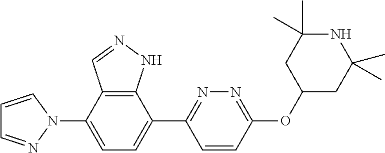 [SMILES: CC1(C)CC(Oc2ccc(-c3ccc(-n4cccn4)c4cn[nH]c34)nn2)CC(C)(C)N1] |
| 152 | 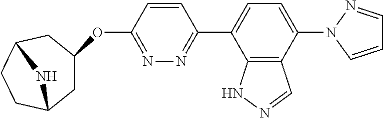 [SMILES: c1cnn(-c2ccc(-c3ccc(O[C@@H]4C[C@H]5CC[C@@H](C4)N5)nn3)c3[nH]ncc23)c1] |
| 153 | 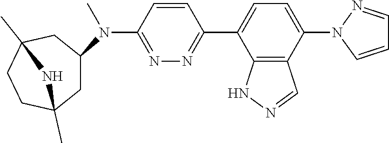 [SMILES: CN(c1ccc(-c2ccc(-n3cccn3)c3cn[nH]c23)nn1)[C@H]1C[C@]2(C)CC[C@](C)(C1)N2] |
| 154 | 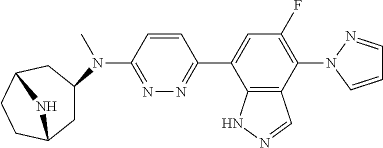 [SMILES: CN(c1ccc(-c2cc(F)c(-n3cccn3)c3cn[nH]c23)nn1)[C@@H]1C[C@H]2CC[C@@H](C1)N2] |
| 155 | 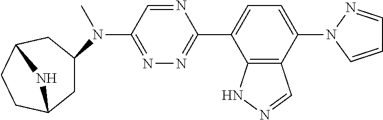 [SMILES: CN(c1cnc(-c2ccc(-n3cccn3)c3cn[nH]c23)nn1)[C@@H]1C[C@H]2CC[C@@H](C1)N2] |
| 156 | 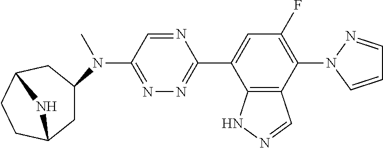 [SMILES: CN(c1cnc(-c2cc(F)c(-n3cccn3)c3cn[nH]c23)nn1)[C@@H]1C[C@H]2CC[C@@H](C1)N2] |
| 157 | 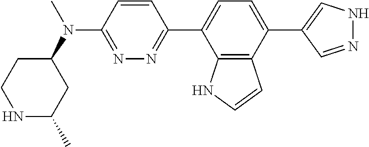 [SMILES: C[C@H]1C[C@H](N(C)c2ccc(-c3ccc(-c4cn[nH]c4)c4cc[nH]c34)nn2)CCN1] |
| 158 | 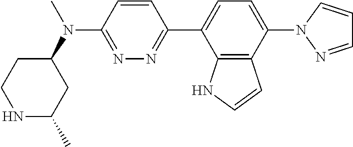 [SMILES: C[C@H]1C[C@H](N(C)c2ccc(-c3ccc(-n4cccn4)c4cc[nH]c34)nn2)CCN1] |
| 159 | 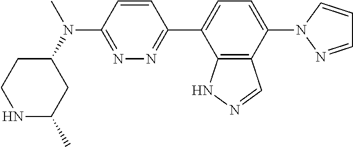 [SMILES: C[C@H]1C[C@@H](N(C)c2ccc(-c3ccc(-n4cccn4)c4cn[nH]c34)nn2)CCN1] |
| 160 | 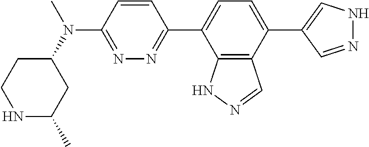 [SMILES: C[C@H]1C[C@@H](N(C)c2ccc(-c3ccc(-c4cn[nH]c4)c4cn[nH]c34)nn2)CCN1] |
| 163 | 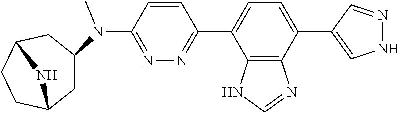 [SMILES: CN(c1ccc(-c2ccc(-c3cn[nH]c3)c3nc[nH]c23)nn1)[C@@H]1C[C@H]2CC[C@@H](C1)N2] |
| 164 | 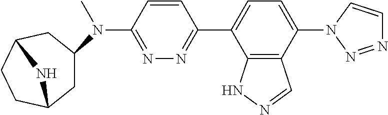 [SMILES: CN(c1ccc(-c2ccc(-n3ccnn3)c3cn[nH]c23)nn1)[C@@H]1C[C@H]2CC[C@@H](C1)N2] |
| 165 | 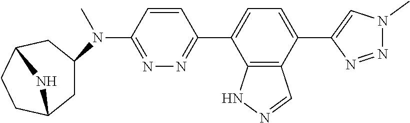 [SMILES: CN(c1ccc(-c2ccc(-c3cn(C)nn3)c3cn[nH]c23)nn1)[C@@H]1C[C@H]2CC[C@@H](C1)N2] |
| 166 | 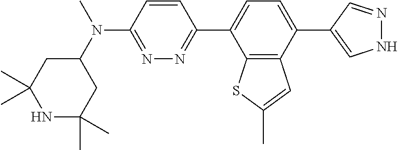 [SMILES: Cc1cc2c(-c3cn[nH]c3)ccc(-c3ccc(N(C)C4CC(C)(C)NC(C)(C)C4)nn3)c2s1] |
| 167 | 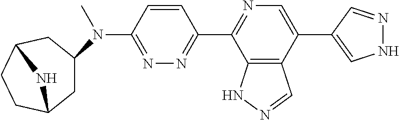 [SMILES: CN(c1ccc(-c2ncc(-c3cn[nH]c3)c3cn[nH]c23)nn1)[C@@H]1C[C@H]2CC[C@@H](C1)N2] |
| 168 | 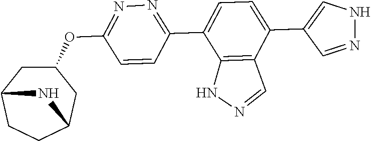 [SMILES: c1n[nH]cc1-c1ccc(-c2ccc(O[C@H]3C[C@H]4CC[C@@H](C3)N4)nn2)c2[nH]ncc12] |
| 169 | 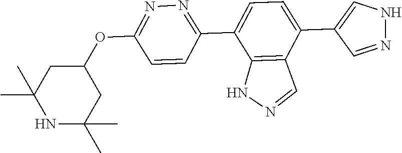 [SMILES: CC1(C)CC(Oc2ccc(-c3ccc(-c4cn[nH]c4)c4cn[nH]c34)nn2)CC(C)(C)N1] |
| 170 | 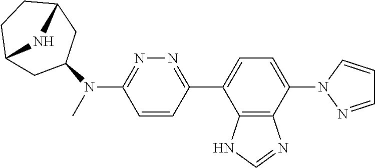 [SMILES: CN(c1ccc(-c2ccc(-n3cccn3)c3nc[nH]c23)nn1)[C@@H]1C[C@H]2CC[C@@H](C1)N2] |
| 174 | 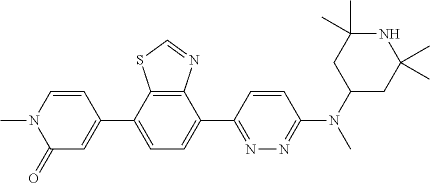 |
| 175 | 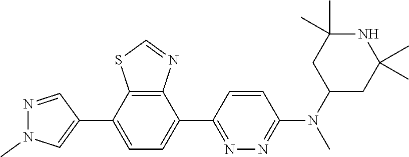 |
| 176 | 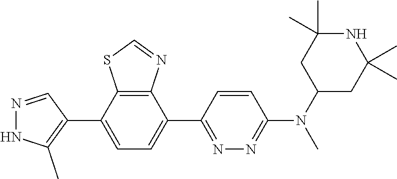 [SMILES: Cc1[nH]ncc1-c1ccc(-c2ccc(N(C)C3CC(C)(C)NC(C)(C)C3)nn2)c2ncsc12] |
| 177 | 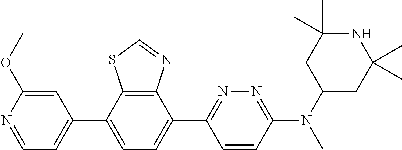 [SMILES: COc1cc(-c2ccc(-c3ccc(N(C)C4CC(C)(C)NC(C)(C)C4)nn3)c3ncsc23)ccn1] |
| 178 | 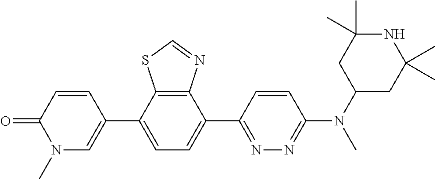 [SMILES: CN(c1ccc(-c2ccc(-c3ccc(=O)n(C)c3)c3scnc23)nn1)C1CC(C)(C)NC(C)(C)C1] |
| 179 | 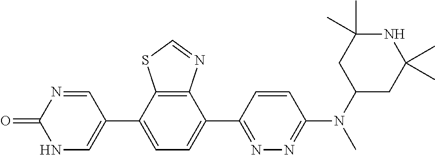 [SMILES: CN(c1ccc(-c2ccc(-c3cnc(=O)[nH]c3)c3scnc23)nn1)C1CC(C)(C)NC(C)(C)C1] |
| 180 | 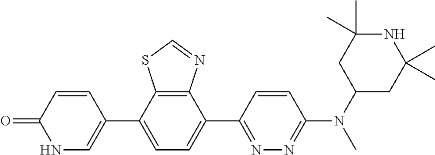 [SMILES: CN(c1ccc(-c2ccc(-c3ccc(=O)[nH]c3)c3scnc23)nn1)C1CC(C)(C)NC(C)(C)C1] |
| 181 | 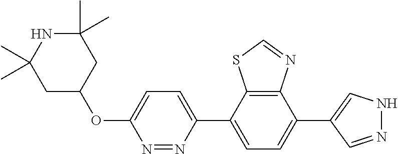 [SMILES: CC1(C)CC(Oc2ccc(-c3ccc(-c4cn[nH]c4)c4ncsc34)nn2)CC(C)(C)N1] |
| 182 | 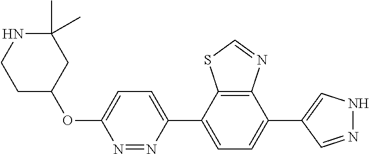 [SMILES: CC1(C)CC(Oc2ccc(-c3ccc(-c4cn[nH]c4)c4ncsc34)nn2)CCN1] |
| 183 | 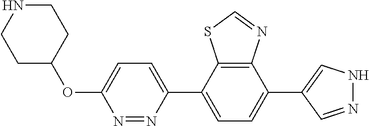 [SMILES: c1nc2c(-c3cn[nH]c3)ccc(-c3ccc(OC4CCNCC4)nn3)c2s1] |
| 184 | 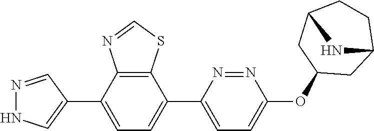 [SMILES: c1nc2c(-c3cn[nH]c3)ccc(-c3ccc(O[C@@H]4C[C@H]5CC[C@@H](C4)N5)nn3)c2s1] |
| 185 | 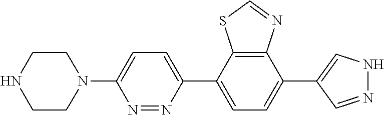 [SMILES: c1nc2c(-c3cn[nH]c3)ccc(-c3ccc(N4CCNCC4)nn3)c2s1] |
| 186 | 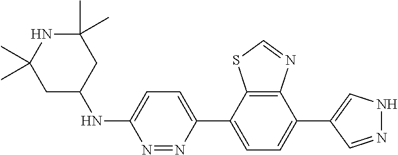 [SMILES: CC1(C)CC(Nc2ccc(-c3ccc(-c4cn[nH]c4)c4ncsc34)nn2)CC(C)(C)N1] |
| 187 | 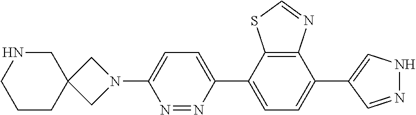 [SMILES: c1nc2c(-c3cn[nH]c3)ccc(-c3ccc(N4CC5(CCCNC5)C4)nn3)c2s1] |
| 188 | 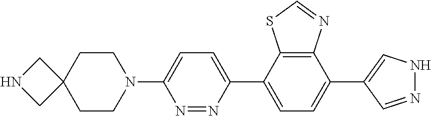 [SMILES: c1nc2c(-c3cn[nH]c3)ccc(-c3ccc(N4CCC5(CC4)CNC5)nn3)c2s1] |
| 189 | 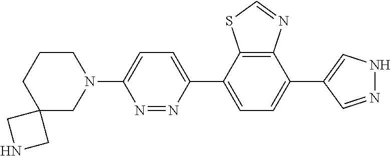 [SMILES: c1nc2c(-c3cn[nH]c3)ccc(-c3ccc(N4CCCC5(CNC5)C4)nn3)c2s1] |
| 190 | 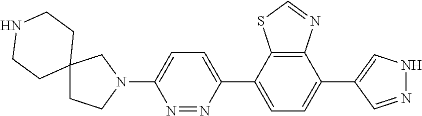 [SMILES: c1nc2c(-c3cn[nH]c3)ccc(-c3ccc(N4CCC5(CCNCC5)C4)nn3)c2s1] |
| 191 | 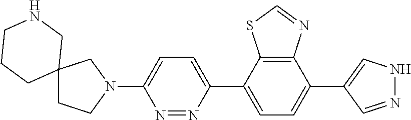 [SMILES: c1nc2c(-c3cn[nH]c3)ccc(-c3ccc(N4CCC5(CCCNC5)C4)nn3)c2s1] |
| 192 | 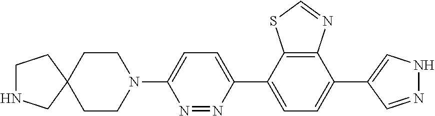 [SMILES: c1nc2c(-c3cn[nH]c3)ccc(-c3ccc(N4CCC5(CCNC5)CC4)nn3)c2s1] |
| 193 | 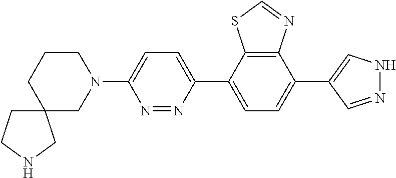 [SMILES: c1nc2c(-c3cn[nH]c3)ccc(-c3ccc(N4CCCC5(CCNC5)C4)nn3)c2s1] |
| 194 | 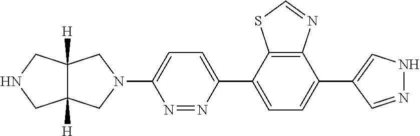 [SMILES: c1nc2c(-c3cn[nH]c3)ccc(-c3ccc(N4C[C@H]5CNC[C@H]5C4)nn3)c2s1] |
| 196 | 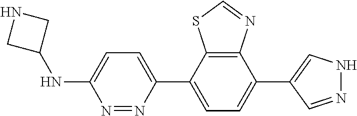 [SMILES: c1nc2c(-c3cn[nH]c3)ccc(-c3ccc(NC4CNC4)nn3)c2s1] |
| 197 | 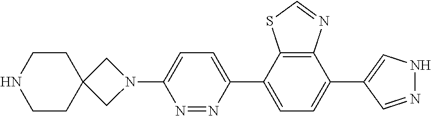 [SMILES: c1nc2c(-c3cn[nH]c3)ccc(-c3ccc(N4CC5(CCNCC5)C4)nn3)c2s1] |
| 198 | 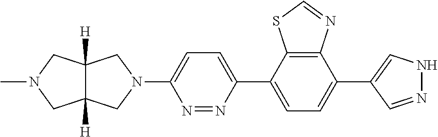 [SMILES: CN1C[C@@H]2CN(c3ccc(-c4ccc(-c5cn[nH]c5)c5ncsc45)nn3)C[C@@H]2C1] |
| 199 | 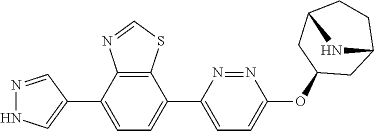 [SMILES: c1nc2c(-c3cn[nH]c3)ccc(-c3ccc(O[C@@H]4C[C@H]5CC[C@@H](C4)N5)nn3)c2s1] |
| 200 | 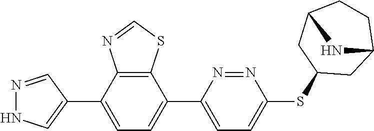 [SMILES: c1nc2c(-c3cn[nH]c3)ccc(-c3ccc(S[C@@H]4C[C@H]5CC[C@@H](C4)N5)nn3)c2s1] |
| 201 | 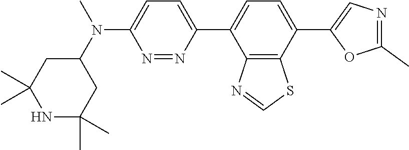 |
| 202 | 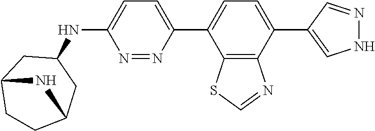 [SMILES: c1nc2c(-c3cn[nH]c3)ccc(-c3ccc(N[C@@H]4C[C@H]5CC[C@@H](C4)N5)nn3)c2s1] |
| 203 | 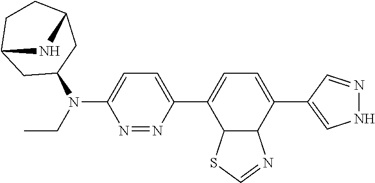 [SMILES: CCN(c1ccc(C2=CC=C(c3cn[nH]c3)C3N=CSC23)nn1)[C@@H]1C[C@H]2CC[C@@H](C1)N2] |
| 204 | 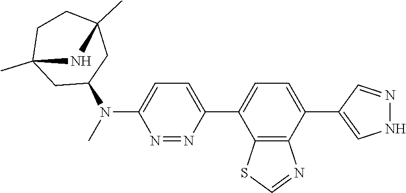 [SMILES: CN(c1ccc(-c2ccc(-c3cn[nH]c3)c3ncsc23)nn1)[C@H]1C[C@]2(C)CC[C@](C)(C1)N2] |
| 205 | 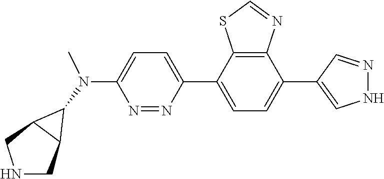 [SMILES: CN(c1ccc(-c2ccc(-c3cn[nH]c3)c3ncsc23)nn1)[C@H]1[C@@H]2CNC[C@@H]21] |
| 206 | 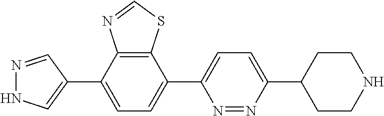 [SMILES: c1nc2c(-c3cn[nH]c3)ccc(-c3ccc(C4CCNCC4)nn3)c2s1] |
| 209 | 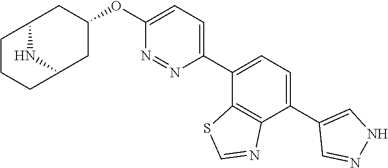 [SMILES: c1nc2c(-c3cn[nH]c3)ccc(-c3ccc(O[C@@H]4C[C@H]5CCC[C@@H](C4)N5)nn3)c2s1] |
| 210 | 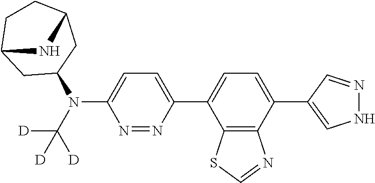 [SMILES: [2H]C([2H])([2H])N(c1ccc(-c2ccc(-c3cn[nH]c3)c3ncsc23)nn1)[C@@H]1C[C@H]2CC[C@@H](C1)N2] |
| 211 | 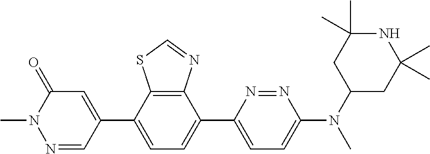 |
| 212 | 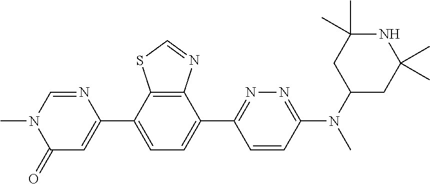 |
| 213 | 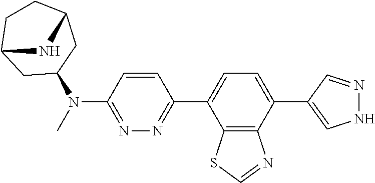 [SMILES: CN(c1ccc(-c2ccc(-c3cn[nH]c3)c3ncsc23)nn1)[C@@H]1C[C@H]2CC[C@@H](C1)N2] |
| 214 | 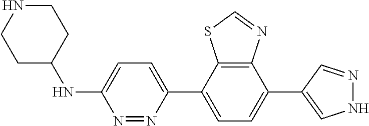 [SMILES: c1nc2c(-c3cn[nH]c3)ccc(-c3ccc(NC4CCNCC4)nn3)c2s1] |
| 215 | 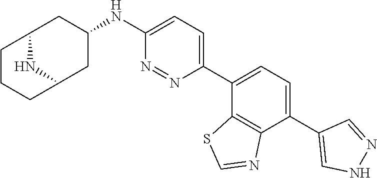 [SMILES: c1nc2c(-c3cn[nH]c3)ccc(-c3ccc(N[C@@H]4C[C@H]5CCC[C@@H](C4)N5)nn3)c2s1] |
| 216 | 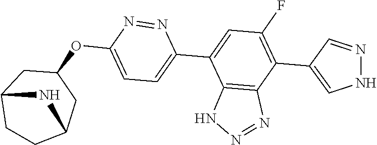 [SMILES: Fc1cc(-c2ccc(O[C@@H]3C[C@H]4CC[C@@H](C3)N4)nn2)c2[nH]nnc2c1-c1cn[nH]c1] |
| 217 | 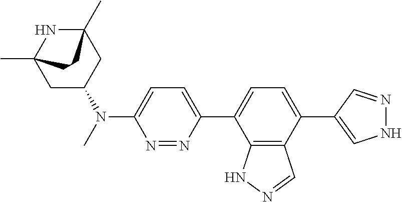 [SMILES: CN(c1ccc(-c2ccc(-c3cn[nH]c3)c3cn[nH]c23)nn1)[C@H]1C[C@]2(C)CC[C@](C)(C1)N2] |
| 218 | 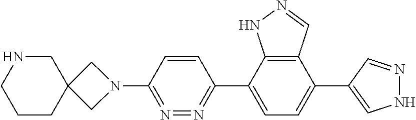 |
| 219 | 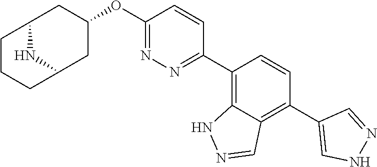 [SMILES: c1n[nH]cc1-c1ccc(-c2ccc(O[C@@H]3C[C@H]4CCC[C@@H](C3)N4)nn2)c2[nH]ncc12] |
| 220 | 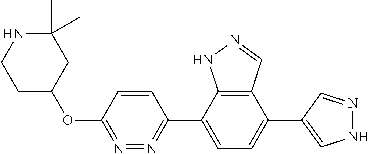 |
| 221 | 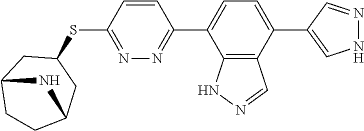 [SMILES: c1n[nH]cc1-c1ccc(-c2ccc(S[C@@H]3C[C@H]4CC[C@@H](C3)N4)nn2)c2[nH]ncc12] |
| 222 | 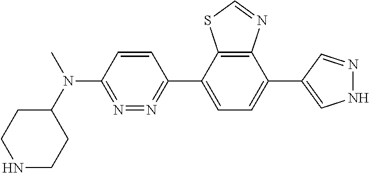 |
| 223 | 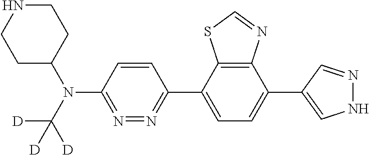 |
| 224 | 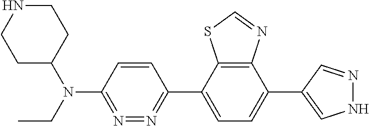 |
| 225 | 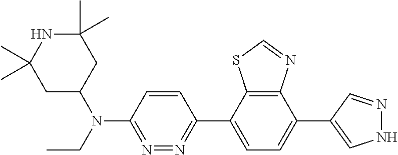 |
| 226 | 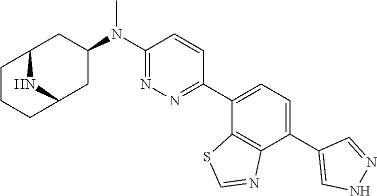 [SMILES: CN(c1ccc(-c2ccc(-c3cn[nH]c3)c3ncsc23)nn1)[C@@H]1C[C@H]2CCC[C@@H](C1)N2] |
| 227 | 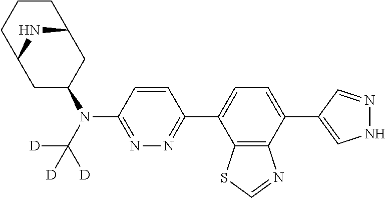 [SMILES: [2H]C([2H])([2H])N(c1ccc(-c2ccc(-c3cn[nH]c3)c3ncsc23)nn1)[C@@H]1C[C@H]2CCC[C@@H](C1)N2] |
| 228 | 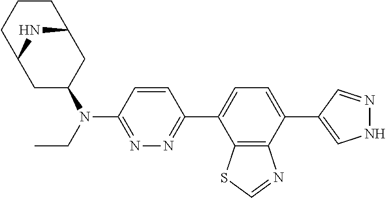 [SMILES: CCN(c1ccc(-c2ccc(-c3cn[nH]c3)c3ncsc23)nn1)[C@@H]1C[C@H]2CCC[C@@H](C1)N2] |
| 229 | 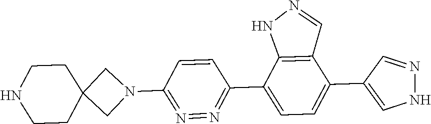 |
| 230 | 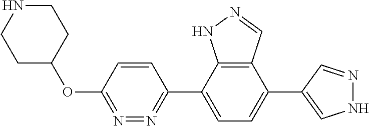 |
| 231 | 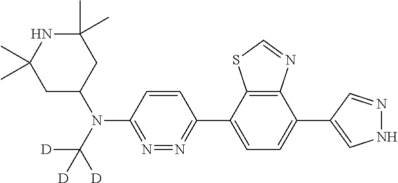 |
| 232 | 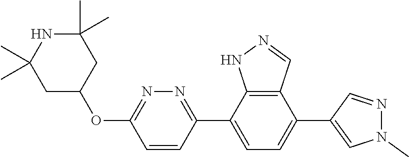 |
| 233 | 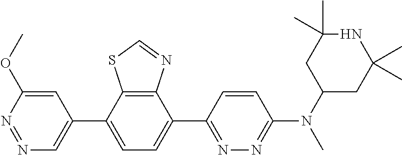 [SMILES: COc1cc(-c2ccc(-c3ccc(N(C)C4CC(C)(C)NC(C)(C)C4)nn3)c3ncsc23)cnn1] |
| 234 | 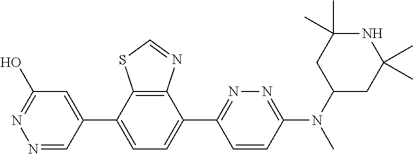 [SMILES: CN(c1ccc(-c2ccc(-c3cnnc(O)c3)c3scnc23)nn1)C1CC(C)(C)NC(C)(C)C1] |
| 235 | 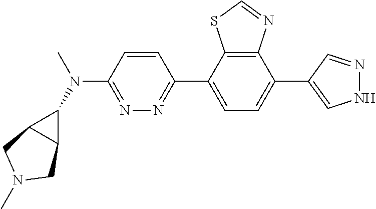 [SMILES: CN1C[C@@H]2[C@H](C1)[C@H]2N(C)c1ccc(-c2ccc(-c3cn[nH]c3)c3ncsc23)nn1] |
| 237 | 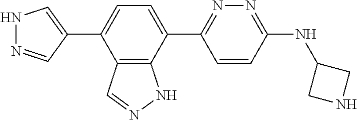 [SMILES: c1n[nH]cc1-c1ccc(-c2ccc(NC3CNC3)nn2)c2[nH]ncc12] |
| 238 | 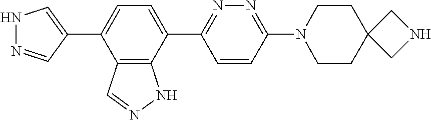 [SMILES: c1n[nH]cc1-c1ccc(-c2ccc(N3CCC4(CC3)CNC4)nn2)c2[nH]ncc12] |
| 239 | 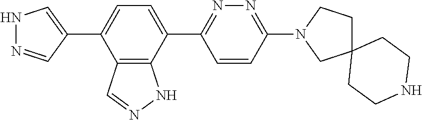 [SMILES: c1n[nH]cc1-c1ccc(-c2ccc(N3CCC4(CCNCC4)C3)nn2)c2[nH]ncc12] |
| 240 | 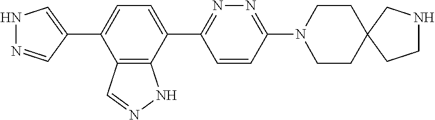 [SMILES: c1n[nH]cc1-c1ccc(-c2ccc(N3CCC4(CCNC4)CC3)nn2)c2[nH]ncc12] |
| 241 | 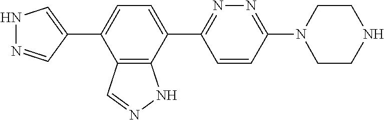 [SMILES: c1n[nH]cc1-c1ccc(-c2ccc(N3CCNCC3)nn2)c2[nH]ncc12] |
| 242 | 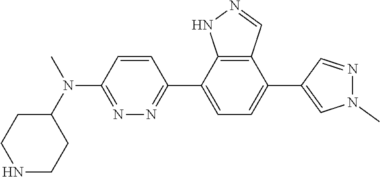 [SMILES: CN(c1ccc(-c2ccc(-c3cnn(C)c3)c3cn[nH]c23)nn1)C1CCNCC1] |
| 243 | 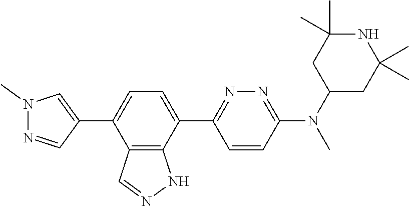 |
| 251 | 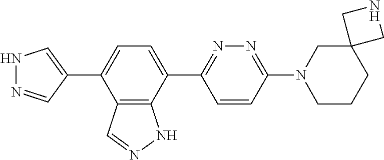 [SMILES: c1n[nH]cc1-c1ccc(-c2ccc(N3CCCC4(CNC4)C3)nn2)c2[nH]ncc12] |
| 252 | 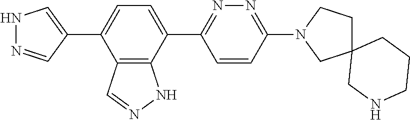 [SMILES: c1n[nH]cc1-c1ccc(-c2ccc(N3CCC4(CCCNC4)C3)nn2)c2[nH]ncc12] |
| 253 | 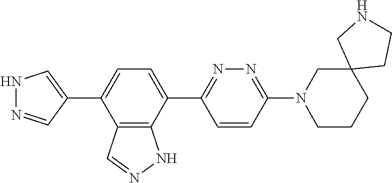 [SMILES: c1n[nH]cc1-c1ccc(-c2ccc(N3CCCC4(CCNC4)C3)nn2)c2[nH]ncc12] |
| 254 | 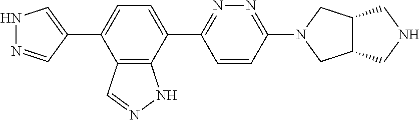 [SMILES: c1n[nH]cc1-c1ccc(-c2ccc(N3C[C@H]4CNC[C@H]4C3)nn2)c2[nH]ncc12] |
| 255 | 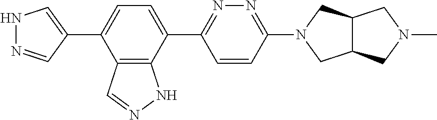 [SMILES: CN1C[C@@H]2CN(c3ccc(-c4ccc(-c5cn[nH]c5)c5cn[nH]c45)nn3)C[C@@H]2C1] |
| 263 | 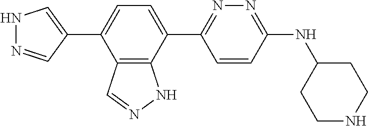 [SMILES: c1n[nH]cc1-c1ccc(-c2ccc(NC3CCNCC3)nn2)c2[nH]ncc12] |
| 264 | 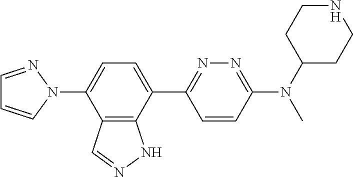 [SMILES: CN(c1ccc(-c2ccc(-n3cccn3)c3cn[nH]c23)nn1)C1CCNCC1] |
| 265 | 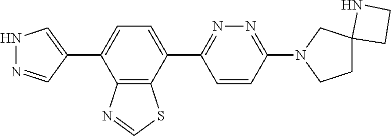 [SMILES: c1nc2c(-c3cn[nH]c3)ccc(-c3ccc(N4CCC5(CCN5)C4)nn3)c2s1] |
| 266 | 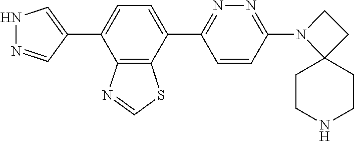 [SMILES: c1nc2c(-c3cn[nH]c3)ccc(-c3ccc(N4CCC45CCNCC5)nn3)c2s1] |
| 267 | 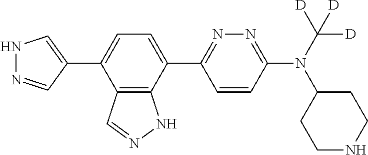 [SMILES: [2H]C([2H])([2H])N(c1ccc(-c2ccc(-c3cn[nH]c3)c3cn[nH]c23)nn1)C1CCNCC1] |
| 273 | 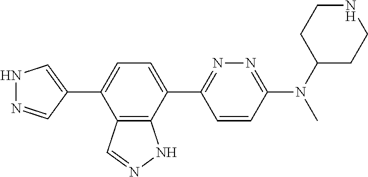 [SMILES: CN(c1ccc(-c2ccc(-c3cn[nH]c3)c3cn[nH]c23)nn1)C1CCNCC1] |
| 274 | 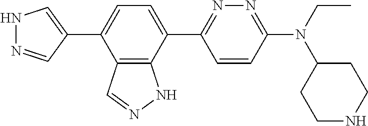 [SMILES: CCN(c1ccc(-c2ccc(-c3cn[nH]c3)c3cn[nH]c23)nn1)C1CCNCC1] |
| 275 | 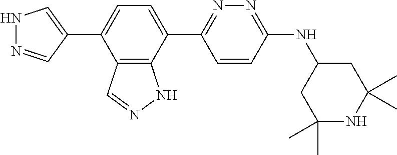 [SMILES: CC1(C)CC(Nc2ccc(-c3ccc(-c4cn[nH]c4)c4cn[nH]c34)nn2)CC(C)(C)N1] |
| 276 | 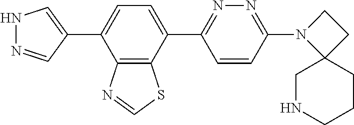 [SMILES: c1nc2c(-c3cn[nH]c3)ccc(-c3ccc(N4CCC45CCCNC5)nn3)c2s1] |
| 277 | 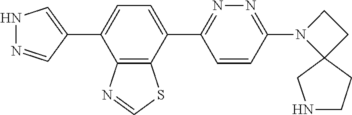 [SMILES: c1nc2c(-c3cn[nH]c3)ccc(-c3ccc(N4CCC45CCNC5)nn3)c2s1] |
| 278 | 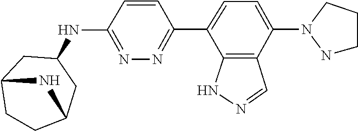 [SMILES: c1cc(-c2ccc(N3CCCN3)c3cn[nH]c23)nnc1N[C@@H]1C[C@H]2CC[C@@H](C1)N2] |
| 279 | 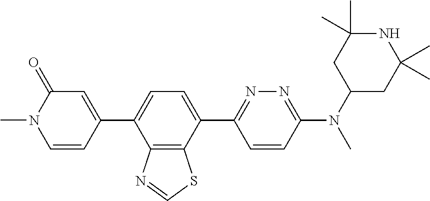 |
| 280 | 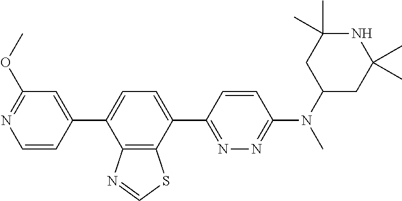 [SMILES: COc1cc(-c2ccc(-c3ccc(N(C)C4CC(C)(C)NC(C)(C)C4)nn3)c3scnc23)ccn1] |
| 281 | 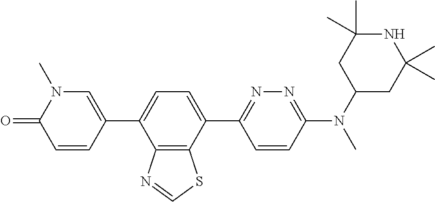 [SMILES: CN(c1ccc(-c2ccc(-c3ccc(=O)n(C)c3)c3ncsc23)nn1)C1CC(C)(C)NC(C)(C)C1] |
| 282 | 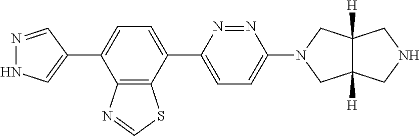 [SMILES: c1nc2c(-c3cn[nH]c3)ccc(-c3ccc(N4C[C@H]5CNC[C@H]5C4)nn3)c2s1] |
| 283 | 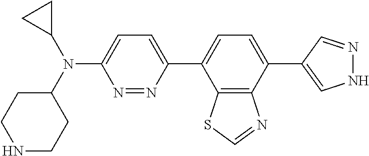 [SMILES: c1nc2c(-c3cn[nH]c3)ccc(-c3ccc(N(C4CCNCC4)C4CC4)nn3)c2s1] |
| 284 | 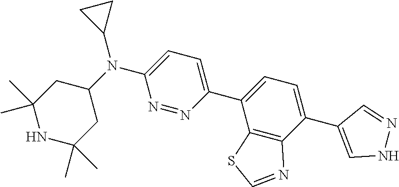 [SMILES: CC1(C)CC(N(c2ccc(-c3ccc(-c4cn[nH]c4)c4ncsc34)nn2)C2CC2)CC(C)(C)N1] |
| 285 | 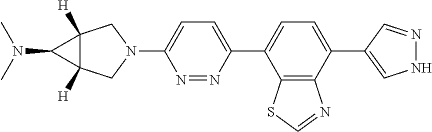 [SMILES: CN(C)[C@H]1[C@@H]2CN(c3ccc(-c4ccc(-c5cn[nH]c5)c5ncsc45)nn3)C[C@@H]21] |
| 286 | 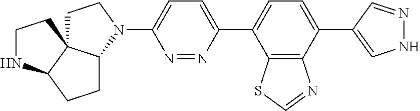 [SMILES: c1nc2c(-c3cn[nH]c3)ccc(-c3ccc(N4CC[C@@]56CCN[C@@H]5CC[C@@H]46)nn3)c2s1] |
| 287 | 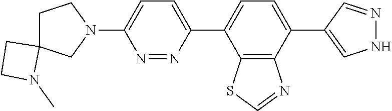 [SMILES: CN1CCC12CCN(c1ccc(-c3ccc(-c4cn[nH]c4)c4ncsc34)nn1)C2] |
| 288 | 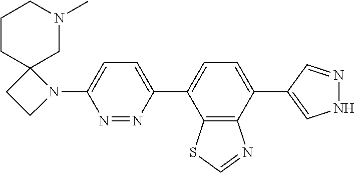 [SMILES: CN1CCCC2(CCN2c2ccc(-c3ccc(-c4cn[nH]c4)c4ncsc34)nn2)C1] |
| 289 | 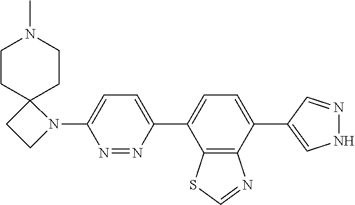 [SMILES: CN1CCC2(CC1)CCN2c1ccc(-c2ccc(-c3cn[nH]c3)c3ncsc23)nn1] |
| 290 | 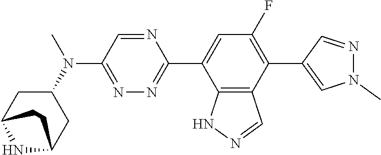 |
| 291 | 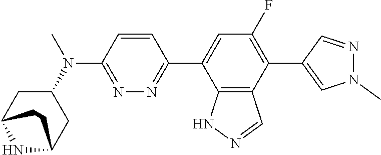 |
| 292 | 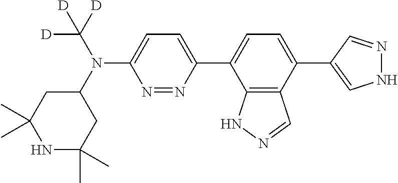 [SMILES: [2H]C([2H])([2H])N(c1ccc(-c2ccc(-c3cn[nH]c3)c3cn[nH]c23)nn1)C1CC(C)(C)NC(C)(C)C1] |
| 293 | 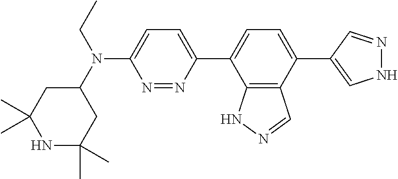 [SMILES: CCN(c1ccc(-c2ccc(-c3cn[nH]c3)c3cn[nH]c23)nn1)C1CC(C)(C)NC(C)(C)C1] |
| 294 | 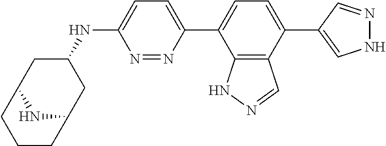 [SMILES: c1n[nH]cc1-c1ccc(-c2ccc(N[C@@H]3C[C@H]4CCC[C@@H](C3)N4)nn2)c2[nH]ncc12] |
| 295 | 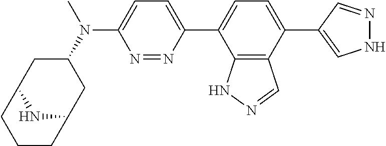 [SMILES: CN(c1ccc(-c2ccc(-c3cn[nH]c3)c3cn[nH]c23)nn1)[C@@H]1C[C@H]2CCC[C@@H](C1)N2] |
| 296 | 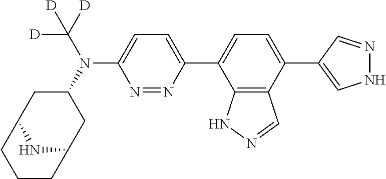 [SMILES: [2H]C([2H])([2H])N(c1ccc(-c2ccc(-c3cn[nH]c3)c3cn[nH]c23)nn1)[C@@H]1C[C@H]2CCC[C@@H](C1)N2] |
| 297 | 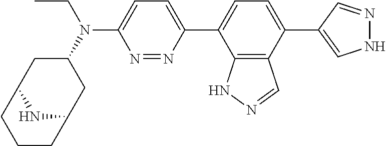 [SMILES: CCN(c1ccc(-c2ccc(-c3cn[nH]c3)c3cn[nH]c23)nn1)[C@@H]1C[C@H]2CCC[C@@H](C1)N2] |
| 298 | 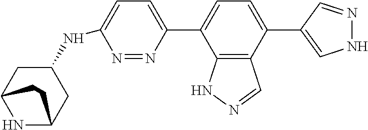 |
| 299 | 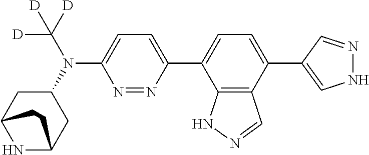 |
| 300 | 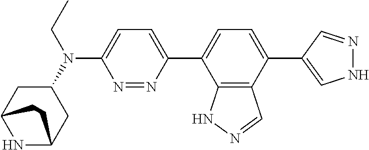 |
| 301 | 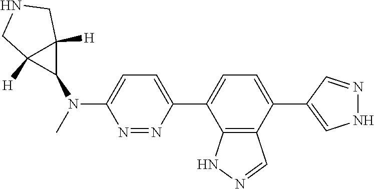 [SMILES: CN(c1ccc(-c2ccc(-c3cn[nH]c3)c3cn[nH]c23)nn1)[C@H]1[C@@H]2CNC[C@@H]21] |
| 302 | 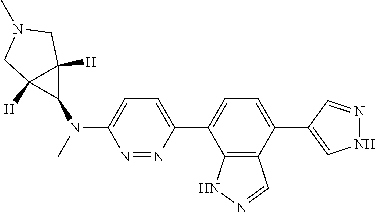 [SMILES: CN1C[C@@H]2[C@H](C1)[C@H]2N(C)c1ccc(-c2ccc(-c3cn[nH]c3)c3cn[nH]c23)nn1] |
| 303 | 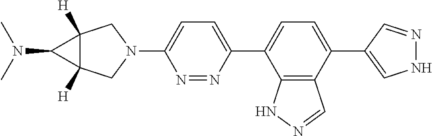 [SMILES: CN(C)[C@H]1[C@@H]2CN(c3ccc(-c4ccc(-c5cn[nH]c5)c5cn[nH]c45)nn3)C[C@@H]21] |
| 304 | 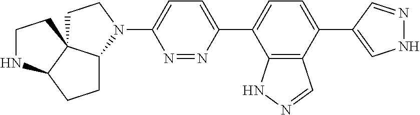 [SMILES: c1n[nH]cc1-c1ccc(-c2ccc(N3CC[C@@]45CCN[C@@H]4CC[C@@H]35)nn2)c2[nH]ncc12] |
| 305 | 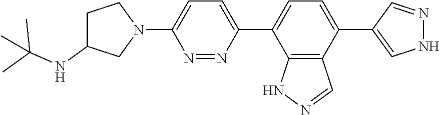 [SMILES: CC(C)(C)NC1CCN(c2ccc(-c3ccc(-c4cn[nH]c4)c4cn[nH]c34)nn2)C1] |
| 306 | 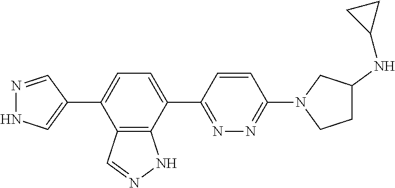 [SMILES: c1n[nH]cc1-c1ccc(-c2ccc(N3CCC(NC4CC4)C3)nn2)c2[nH]ncc12] |
| 307 | 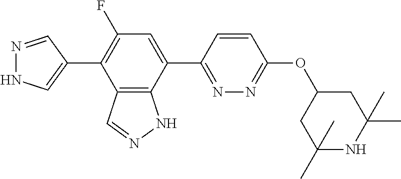 |
| 308 | 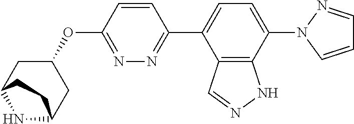 |
| 309 | 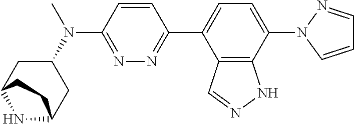 |
| 310 | 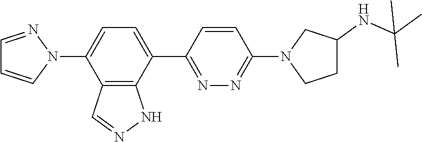 [SMILES: CC(C)(C)NC1CCN(c2ccc(-c3ccc(-n4cccn4)c4cn[nH]c34)nn2)C1] |
| 311 | 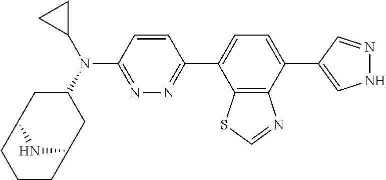 |

### Pharmaceutical Compositions, Kits, and Administration

The present invention provides pharmaceutical compositions comprising a compound of Formula (I) e.g., a compound of Formula (I) or a pharmaceutically acceptable salt, solvate, hydrate, tautomer, or stereoisomer, as described herein, and optionally a pharmaceutically acceptable excipient. In certain embodiments, the pharmaceutical composition described herein comprises a compound of Formula (I) or a pharmaceutically acceptable salt thereof, and optionally a pharmaceutically acceptable excipient. In certain embodiments, the compound of Formula (I) or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

Thus, in one aspect, provided are kits including a first container comprising a compound described herein, or a pharmaceutically acceptable salt, solvate, hydrate, tautomer, or stereoisomer thereof, or a pharmaceutical composition thereof. In certain embodiments, the kit of the disclosure includes a first container comprising a compound described herein, or a pharmaceutically acceptable salt thereof, or a pharmaceutical composition thereof. In certain embodiments, the kits are useful in preventing and/or treating a disease, disorder, or condition described herein in a subject (e.g., a proliferative disease or a non-proliferative disease). In certain embodiments, the kits further include instructions for administering the compound, or a pharmaceutically acceptable salt, solvate, hydrate, tautomer, or stereoisomer thereof, or a pharmaceutical composition thereof, to a subject to prevent and/or treat a proliferative disease or a non-proliferative disease.

### Methods of Use

Described herein are compounds useful for modulating splicing. In some embodiments, a compound of Formula (I) may be used to alter the amount, structure, or composition of a nucleic acid (e.g., a precursor RNA, e.g., a pre-mRNA, or the resulting mRNA) by increasing or decreasing splicing at a splice site. In some embodiments, increasing or decreasing splicing results in modulating the level or structure of a gene product (e.g., an RNA or protein) produced. In some embodiments, a compound of Formula (I) may modulate a component of the splicing machinery, e.g., by modulating the interaction with a component of the splicing machinery with another entity (e.g., nucleic acid, protein, or a combination thereof). The splicing machinery as referred to herein comprises one or more spliceosome components. Spliceosome components may comprise, for example, one or more of major spliceosome members (U1, U2, U4, U5, U6 snRNPs), or minor spliceosome members (U11, U12, U4atac, U6atac snRNPs) and their accessory splicing factors.

In another aspect, the present disclosure features a method of modifying of a target (e.g., a precursor RNA, e.g., a pre-mRNA) through inclusion of a splice site in the target, wherein the method comprises providing a compound of Formula (I). In some embodiments, inclusion of a splice site in a target (e.g., a precursor RNA, e.g., a pre-mRNA, or the resulting mRNA) results in addition or deletion of one or more nucleic acids to the target (e.g., a new exon, e.g. a skipped exon). Addition or deletion of one or more nucleic acids to the target may result in an increase in the levels of a gene product (e.g., RNA, e.g., mRNA, or protein).

In another aspect, the present disclosure features a method of modifying a target (e.g., a precursor RNA, e.g., a pre-mRNA, or the resulting mRNA) through exclusion of a splice site in the target, wherein the method comprises providing a compound of Formula (I). In some embodiments, exclusion of a splice site in a target (e.g., a precursor RNA, e.g., a pre-mRNA) results in deletion or addition of one or more nucleic acids from the target (e.g., a skipped exon, e.g. a new exon). Deletion or addition of one or more nucleic acids from the target may result in a decrease in the levels of a gene product (e.g., RNA, e.g., mRNA, or protein). In other embodiments, the methods of modifying a target (e.g., a precursor RNA, e.g., a pre-mRNA, or the resulting mRNA) comprise suppression of splicing at a splice site or enhancement of splicing at a splice site (e.g., by more than about 0.5%, e.g., 1%, 5%, 10%, 15%, 20%, 25%, 30%, 35%, 40%, 45%, 50%, 55%, 60%, 65%, 70%, 75%, 80%, 85%, 90%, 95%, 99%, or more), e.g., as compared to a reference (e.g., the absence of a compound of Formula (I), or in a healthy or diseased cell or tissue).

The methods described herein can be used to modulate splicing, e.g., of a nucleic acid comprising a particular sequence (e.g., a target sequence). Exemplary genes encoding a target sequence (e.g., a target sequence comprising DNA or RNA, e.g., pre-mRNA) include, inter alia, ABCA4, ABCA9, ABCB1, ABCB5, ABCC9, ABCD1, ACADL, ACADM, ACADSB, ACSS2, ACTB, ACTG2, ADA, ADAL, ADAM10, ADAM15, ADAM22, ADAM32, ADAMTS12, ADAMTS13, ADAMTS20, ADAMTS6, ADAMTS9, ADAR, ADCY3, ADCY10, ADCY8, ADNP, ADRBK2, AFP, AGL, AGT, AHCTF1, AHR, AKAP10, AKAP3, AKNA, ALAS1, ALS2CL, ALB, ALDH3A2, ALG6, AMBRA1, ANK3, ANTXR2, ANXA10, ANXA11, ANGPTL3, AP2A2, AP4E1, APC, APOA1, APOB, APOC3, APOH, AR, ARID2, ARID3A, ARID3B, ARFGEF1, ARFGEF2, ARHGAP1, ARHGAP8, ARHGAP18, ARHGAP26, ARHGEF18, ARHGEF2, ARPC3, ARS2, ASH1L, ASH1L-IT1, ASNSD1, ASPM, ATAD5, ATF1, ATG4A, ATG16L2, ATM, ATN1, ATP11C, ATP6V1G3, ATP13A5, ATP7A, ATP7B, ATR, ATXN2, ATXN3, ATXN7, ATXN10, AXIN1, B2M, B4GALNT3, BBS4, BCL2, BCL2L1, BCL2-like 11 (BIM), BCL11B, BBOX1, BCS1L, BEAN1, BHLHE40, BMPR2, BMP2K, BPTF, BRAF, BRCA1, BRCA2, BRCC3, BRSK1, BRSK2, BTAF1, BTK, C2orf55, C4orf29, C6orf118, C9orf43, C9orf72, C10orf137, C11orf30, C11orf65, C11orf70, C11orf87, C12orf51, C13orf1, C13orf15, C14orf1l, C14orf118, C15orf29, C15orf42, C15orf60, C16orf33, C16orf38, C16orf48, C18orf8, C19orf42, C1orf107, C1orf114, C1orf130, C1orf149, C1orf27, C1orf71, C1orf94, C1R, C20orf74, C21orf70, C3orf23, C4orf18, C5orf34, C8B, C8orf33, C9orf114, C9orf86, C9orf98, C3, CA11, CAB39, CACHD1, CACNA1A, CACNA1B, CACNA1C, CACNA2D1, CACNA1G, CACNA1H, CALCA, CALCOCO2, CAMK1D, CAMKK1, CAPN3, CAPN9, CAPSL, CARD11, CARKD, CASZ1, CAT, CBLB, CBX1, CBX3, CCDC102B, CCDC11, CCDC15, CCDC18, CCDC5, CCDC81, CCDC131, CCDC146, CD4, CD274, CD1B, CDC14A, CDC16, CDC2L5, CDC42BPB, CDCA8, CDH10, CDH11, CDH24, CDH8, CDH9, CDK5RAP2, CDK6, CDK8, CDK11B, CD33, CD46, CDH1, CDH23, CDK6, CDK11B, CDK13, CEBPZ, CEL, CELSR3, CENPA, CENPI, CENPT, CENTB2, CENTG2, CEPJ10, CEP170, CEP192, CETP, CFB, CFTR, CFH, CGN, CGNL1, CHAF1A, CHD9, CHIC2, CHL1, CHN1, CHM, CLEC16A, CLIC2, CLCN1, CLINT1, CLK1, CLPB, CLPTM1, CMIP, CMYA5, CNGA3, CNOT1, CNOT7, CNTN6, COG3, COL11A1, COL11A2, COL12A1, COL14A1, COL15A1, COL17A1, COL19A1, COL1A1, COL1A2, COL2A1, COL3A1, COL4A1, COL4A2, COL4A5, COL4A6, COL5A2, COL6A1, COL7A1, COL9A, COL9A2, COL22A1, COL24A1, COL25A1, COL29A1, COLQ, COMTD1, COPA, COPB2, COPS7B, COPZ2, CPSF2, CPXM2, CR1, CRBN, CRYZ, CREBBP, CRKRS, CSE1L, CSTB, CSTF3, CT45-6, CTNNB1, CUBN, CUL4B, CUL5, CXorf41, CXXC1, CYBB, CYFIP2, CYP3A4, CYP3A43, CYP3A5, CYP4F2, CYP4F3, CYP17, CYP19, CYP24A1, CYP27A1, DAB1, DAZ2, DCBLD1, DCC, DCTN3, DCUN1D4, DDA1, DDEF1, DDX1, DDX24, DDX4, DENND2D, DEPDC2, DES, DGAT2, DHFR, DHRS7, DHRS9, DHX8, DIP2A, DMD, DMTF, DNAH3, DNAH8, DNAI1, DNAJA4, DNAJC13, DNAJC7, DNMT1, DNTTIP2, DOCK4, DOCK5, DOCK10, DOCK11, DOT1L, DPP3, DPP4, DPY19L2P2, DR1, DSCC1, DVL3, DUX4, DYNC1H1, DYSF, E2F1, E2F3, E2F8, E4F1, EBF1, EBF3, ECM2, EDEM3, EFCAB3, EFCAB4B, EFNA4, EFTUD2, EGFR, EIF3A, ELA1, ELA2A, ELF2, ELF3, ELF4, EMCN, EMD, EML5, ENO3, ENPP3, EP300, EPAS1, EPB41L5, EPHA3, EPHA4, EPHB1, EPHB2, EPHB3, EPS15, ERBB4, ERCC1, ERCC8, ERGIC3, ERMN, ERMP1, ERN1, ERN2, ESR1, ESRRG, ETS2, ETV3, ETV4, ETV5, ETV6, EVC2, EWSR1, EXO1, EXOC4, F3, F11, F13A1, F5, F7, F8, FAH, FAM13A1, FAM13B1, FAM13C1, FAM134A, FAM161A, FAM176B, FAM184A, FAM19A1, FAM20A, FAM23B, FAM65C, FANCA, FANCC, FANCG, FANCM, FANK1, FAR2, FBN1, FBXO15, FBXO18, FBXO38, FCGBP, FECH, FEZ2, FGA, FGD6, FGFR2, FGFRIOP, FGFRIOP2, FGFR2, FGG, FGR, FIX, FKBP3, FLI1, FLJ35848, FLJ36070, FLNA, EN1, FNBP1L, FOLH1, FOSL1, FOSL2, FOXK1, FOXM1, FOXO1, FOXP4, FRAS1, FUT9, FXN, FZD3, FZD6, GAB1, GABPA, GALC, GALNT3, GAPDH, GART, GAS2L3, GATA3, GATAD2A, GBA, GBGT1, GCG, GCGR, GCK, GFI1, GFM1, GH1, GHR, GHV, GJA1, GLA, GLT8D1, GNA11, GNAQ, GNAS, GNB5, GOLGB1, GOLT1A, GOLT1B, GPATCH1, GPR158, GPR160, GPX4, GRAMD3, GRHL1, GRHL2, GRHPR, GRIA1, GRIA3, GRIA4, GRIN2B, GRM3, GRM4, GRN, GSDMB, GSTCD, GSTO2, GTF2I, GTPBP4, HADHA, HAND2, HBA2, HBB, HCK, HDAC3, HDAC5, HDX, HEPACAM2, HERC1, HES7, HEXA, HEXB, HHEX, HIPK3, HLA-DPB1, HLA-G, HLCS, HLTF, HMBS, HMGA1, HMGCL, HNF1A, HNF1B, HNF4A, HNF4G, HNRNPH1, HOXC10, HP1BP3, HPGD, HPRT1, HPRT2, HSF1, HSF4, HSF2BP, HSPA9, HSPG2, HTT, HXA, ICA1, IDH1, IDS, IFI44L, IKBKAP, IKZF1, IKZF3, ILIR2, IL5RA, IL7RA, IMMT, INPP5D, INSR, INTS3, INTU, IP04, IP08, IQGAP2, IRF2, IRF4, IRF8, IRX3, ISL1, ISL2, ITFG1, ITGA6, ITGAL, ITGB1, ITGB2, ITGB3, ITGB4, ITIH1, ITPR2, IWS1, JAK1, JAK2, JAG1, JMJD1C, JPH3, KALRN, KAT6A, KATNAL2, KCNN2, KCNT2, KDM2A, KIAA0256, KIAA0528, KIAA0564, KIAA0586, KIAA1033, KIAA1166, KIAA1219, KIAA1409, KIAA1622, KIAA1787, KIF3B, KIF15, KIF16B, KIF5A, KIF5B, KIF9, KIN, KIR2DL5B, KIR3DL2, KIR3DL3, KIT, KLF3, KLF5, KLF7, KLF0, KLF12, KLF16, KLHL20, KLK12, KLKB1, KMT2A, KMT2B, KPNA5, KRAS, KREMEN1, KRIT1, KRT5, KRTCAP2, KYNU, L1CAM, L3MBTL, L3MBTL2, LACE1, LAMA1, LAMA2, LAMA3, LAMB1, LARP7, LDLR, LEF1, LENG1, LGALS3, LGMN, LHCGR, LHX3, LHX6, LIMCH1, LIMK2, LIN28B, LIN54, LMBRD1, LMBRD2, LMLN, LMNA, LMO2, LMO7, LOC389634, LOC390110, LPA, LPCAT2, LPL, LRP4, LRPPRC, LRRK2, LRRC19, LRRC42, LRWD1, LUM, LVRN, LYN, LYST, MADD, MAGI1, MAGT1, MALT1, MAP2K1, MAP4K4, MAPK8IP3, MAPK9, MAPT, MARC, MARCH5, MATN2, MBD3, MCF2L2, MCM6, MDGA2, MDM4, ASXL1, FUS, SPR54, MECOM, MEF2C, MEF2D, MEGF10, MEGF11, MEMO1, MET, MGA, MGAM, MGAT4A, MGAT5, MGC16169, MGC34774, MKKS, MIB1, MIER2, MITF, MKL2, MLANA, MLH1, MLL5, MLX, MME, MPDZ, MPI, MRAP2, MRPL11, MRPL39, MRPS28, MRPS35, MS4A13, MSH2, MSH3, MSMB, MST1R, MTDH, MTERF3, MTF1, MTF2, MTIF2, MTHFR, MUC2, MUT, MVK, MYB, MYBL2, MYC, MYCBP2, MYH2, MYRF, MYT1, MY019, MY03A, MY09B, MYOM2, MYOM3, NAG, NARG1, NARG2, NCOA1, NDC80, NDFIP2, NEB, NEDD4, NEK1, NEK5, NEK11, NF1, NF2, NFATC2, NFE2L2, NFIA, NFIB, NFIX, NFKB1, NFKB2, NFKBIL2, NFRKB, NFYA, NFYB, NIPA2, NKAIN2, NKAP, NLRC3, NLRC5, NLRP3, NLRP7, NLRP8, NLRP13, NME1, NME1-NME2, NME2, NME7, NOL10, NOP561, NOS1, NOS2A, NOTCH1, NPAS4, NPM1, NRID1, NR1H3, NR1H4, NR4A3, NR5A1, NRXN1, NSMAF, NSMCE2, NT5C, NT5C2, NT5C3, NUBP1, NUBPL, NUDT5, NUMA1, NUP88, NUP98, NUP160, NUPL1, OAT, OAZ1, OBFC2A, OBFC2B, OLIG2, OMA1, OPA1, OPN4, OPTN, OSBPL11, OSBPL8, OSGEPL1, OTC, OTX2, OVOL2, OXT, PA2G4, PADI4, PAH, PAN2, PAOX, PAPOLG, PARD3, PARP1, PARVB, PAWR, PAX3, PAX8, PBGD, PBRM1, PBX2, PCBP4, PCCA, PCGF2, PCNX, PCOTH, PDCD4, PDE4D, PDE8B, PDE10A, PD1A3, PDH1, PDLIM5, PDXK, PDZRN3, PELI2, PDK4, PDS5A, PDS5B, PGK1, PGM2, PHACTR4, PHEX, PHKB, PHLDB2, PHOX2B, PHTF1, PIAS1, PIEZO1, PIGF, PIGN, PIGT, PIK3C2G, PIK3CA, PIK3CD, PIK3CG, PIK3RI, PIP5K1A, PITRM1, PIWIL3, PKD1, PKHD1L1, PKD2, PKIB, PKLR, PKM1, PKM2, PLAGL2, PLCB1, PLCB4, PLCG1, PLD1, PLEKHA5, PLEKHA7, PLEKHM1, PLKR, PLXNC1, PMFBP1, POLN, POLR3D, POMT2, POSTN, POU2AF1, POU2F2, POU2F3, PPARA, PPFIA2, PPPIR12A, PPP3CB, PPP4C, PPP4R1L, PPP4R2, PRAME, PRC1, PRDM1, PREX1, PREX2, PRIM1, PRIM2, PRKAR1A, PRKCA, PRKG1, PRMT7, PROC, PROCR, PROSC, PRODH, PROX1, PRPF40B, PRPF4B, PRRG2, PRUNE2, PSD3, PSEN1, PSMAL, PTCH1, PTEN, PTK2, PTK2B, PTPN2, PTPN3, PTPN4, PTPN11, PTPN22, PTPRD, PTPRK, PTPRM, PTPRN2, PTPRT, PUSIO, PVRL2, PYGM, QRSL1, RAB11FIP2, RAB23, RAF1, RALBP1, RALGDS, RB1CC1, RBL2, RBM39, RBM45, RBPJ, RBSN, REC8, RELB, RFC4, RFTJ, RFTN1, RHOA, RHPN2, RIF1, RIT1, RLN3, RMND5B, RNFI1, RNF32, RNFT1, RNGTT, ROCK1, ROCK2, RORA, RP1, RP6KA3, RP11-265F1, RP13-36C9, RPAP3, RPN1, RPGR, RPL22, RPL22L1, RPS6KA6, RREB1, RRM1, RRP1B, RSK2, RTEL1, RTF1, RUFY1, RUNX1, RUNX2, RXRA, RYR3, SAAL1, SAE1, SALL4, SAT1, SATB2, SBCAD, SCN1A, SCN2A, SCN3A, SCN4A, SCN5A, SCN8A, SCNA, SCN11A, SCO1, SCYL3, SDC1, SDK1, SDK2, SEC24A, SEC24D, SEC31A, SEL1L, SENP3, SENP6, SENP7, SERPINA1, SETD3, SETD4, SETDB1, SEZ6, SFRS12, SGCE, SGOL2, SGPL1, SH2D1A, SH3BGRL2, SH3PXD2A, SH3PXD2B, SH3RF2, SH3TC2, SHOC2, SIPA1L2, SIPA1L3, SIVA1, SKAP1, SKIV2L2, SLC6A11, SLC6A13, SLC6A6, SLC7A2, SLC12A3, SLC13A1, SLC22A17, SLC25A14, SLC28A3, SLC33A1, SLC35F6, SLC38A1, SLC38A4, SLC39A10, SLC4A2, SLC6A8, SMARCA1, SMARCA2, SMARCA5, SMARCC2, SMC5, SMN2, SMOX, SMS, SMTN, SNCAIP, SNORD86, SNRK, SNRP70, SNX5, SNX6, SOD1, SOD10, SOS, SOS2, SOX5, SOX6, SOX8, SP1, SP2, SP3, SP110, SPAG9, SPATA13, SPATA4, SPATS1, SPECC1L, SPDEF, SPI1, SPINK5, SPP2, SPTA1, SRF, SRM, SRP72, SSX3, SSX5, SSX9, STAG1, STAG2, STAMBPLI, STARD6, STAT1, STAT3, STAT5A, STAT5B, STAT6, STK17B, STX3, STXBPJ, SUCLG2, SULF2, SUPT6H, SUPT16H, SV2C, SYCP2, SYT6, SYCPI, SYTL3, SYTL5, TAF2, TARDBP, TBCID3G, TBC1D8B, TBC1D26, TBC1D29, TBCEL, TBK1, TBP, TBPL1, TBR1, TBX, TCEB3, TCF3, TCF4, TCF7L2, TCFL5, TCF12, TCP11L2, TDRD3, TEAD1, TEAD3, TEAD4, TECTB, TEK, TERF1, TERF2, TET2, TFAP2A, TFAP2B, TFAP2C, TFAP4, TFDP1, TFRC, TG, TGM7, TGS1, THAP7, THAP12, THOC2, TIAL1, TIAM2, TIMM50, TLK2, TM4SF20, TM6SF1, TMEM27, TMEM77, TMEM156, TMEM194A, TMF1, TMPRSS6, TNFRSF10A, TNFRSF10B, TNFRSF8, TNK2, TNKS, TNKS2, TOM1L1, TOM1L2, TOP2B, TP53, TP53INP1, TP53BP2, TP53I3, TP63, TRAF3IP3, TRAPPC2, TRIM44, TRIM65, TRIML1, TRIML2, TRPM3, TRPM5, TRPM7, TRPS1, TSC1, TSC2, TSHB, TSPAN7, TTC17, TTF1, TTLL5, TTLL9, TTN, TTPAL, TTR, TUSC3, TXNDC10, UBE3A, UCK, UGTIA1, UHRF1BP1, UNC45B, UNC5C, USH2A, USF2, USP1, USP6, USP18, USP38, USP39, UTP20, UTP15, UTP18, UTRN, UTX, UTY, UVRAG, UXT, VAPA, VEGFA, VPS29, VPS35, VPS39, VT11A, VT11B, VWA3B, WDFY2, WDR16, WDR17, WDR26, WDR44, WDR67, WDTC1, WRN, WRNIP1, WT1, WWC3, XBP1, XRN1, XRN2, XX-FW88277, YAP1, YARS, YBX1, YGM, YY1, ZBTB18, ZBTB20, ZC3HAV1, ZC3HC1, ZC3H7A, ZDHHC19, ZEB1, ZEB2, ZFPM1, ZFYVE1, ZFX, ZIC2, ZNF37A, ZNF91, ZNF114, ZNF155, ZNF169, ZNF205, ZNF236, ZNF317, ZNF320, ZNF326, ZNF335, ZNF365, ZNF367, ZNF407, ZNF468, ZNF506, ZNF511, ZNF511-PRAP1, ZNF519, ZNF521, ZNF592, ZNF618, ZNF763, and ZWINT.

Additional exemplary genes encoding a target sequence (e.g., a target sequence comprising DNA or RNA, e.g., pre-mRNA) include genes include A1CF, A4GALT, AAR2, ABAT, ABCA11P, ZNF721, ABCA5, ABHD10, ABHD13, ABHD2, ABHD6, ACO000120.3, KRIT1, AC004076.1, ZNF772, AC004076.9, ZNF772, AC004223.3, RAD51D, AC004381.6, AC006486.1, ERF, ACO007390.5, ACO007780.1, PRKAR1A, AC007998.2, INO80C, ACO009070.1, CMC2, AC009879.2, AC009879.3, ADHFE1, AC010487.3, ZNF816-ZNF321P, ZNF816, AC010328.3, AC010522.1, ZNF587B, AC010547.4, ZNF19, AC012313.3, ZNF497, AC012651.1, CAPN3, AC013489.1, DET1, AC016747.4, C2orf74, AC020907.6, FXYD3, AC021087.5, PDCD6, AHRR, AC022137.3, ZNF761, AC025283.3, NAA60, AC027644.4, RABGEF1, AC055811.2, FLCN, AC069368.3, ANKDD1A, AC073610.3, ARF3, AC074091.1, GPN1, AC079447.1, LIPT1, AC092587.1, AC079594.2, TRIM59, AC091060.1, C18orf21, AC092143.3, MC1R, AC093227.2, ZNF607, AC093512.2, ALDOA, AC098588.1, ANAPC10, AC107871.1, CALML4, AC114490.2, ZMYM6, AC138649.1, NIPA1, AC138894.1, CLN3, AC139768.1, AC242426.2, CHD1L, ACADM, ACAP3, ACKR2, RP11-141M3.5, KRBOX1, ACMSD, ACOT9, ACP5, ACPL2, ACSBG1, ACSF2, ACSF3, ACSL1, ACSL3, ACVR1, ADAL, ADAM29, ADAMTS10, ADAMTSL5, ADARB1, ADAT2, ADCK3, ADD3, ADGRG1, ADGRG2, ADH1B, ADIPOR1, ADNP, ADPRH, AGBL5, AGPAT1, AGPAT3, AGR2, AGTR1, AHDC1, AHI1, AHNAK, AIFM1, AIFM3, AIMP2, AK4, AKAP1, AKNAD1, CLCC1, AKR1A1, AKT1, AKT1S1, AKT2, AL139011.2, PEX19, AL157935.2, ST6GALNAC6, AL358113.1, TJP2, AL441992.2, KYAT1, AL449266.1, CLCC1, AL590556.3, LINC00339, CDC42, ALAS1, ALB, ALDH16A1, ALDH1B1, ALDH3A1, ALDH3B2, ALDOA, ALKBH2, ALPL, AMD1, AMICA1, AMN1, AMOTL2, AMY1B, AMY2B, ANAPC10, ANAPC11, ANAPC15, ANG, RNASE4, AL163636.2, ANGEL2, ANGPTL1, ANKMY1, ANKRD11, ANKRD28, ANKRD46, ANKRD9, ANKS3, ANKS3, RP11-127I20.7, ANKS6, ANKZF, ANPEP, ANXA11, ANXA2, ANXA8L2, AL603965.1, AOC3, AP000304.12, CRYZL1, AP000311.1, CRYZL1, AP000893.2, RAB30, AP001267.5, ATP5MG, AP002495.2, AP003175.1, OR2AT4, AP003419.1, CLCF1, AP005263.1, ANKRD12, AP006621.5, AP006621.1, AP1G1, AP3M1, AP3M2, APBA2, APBB1, APLP2, APOA2, APOL1, APOL3, APTX, ARAP1, STARD10, ARF4, ARFIP1, ARFIP2, ARFRP1, ARHGAP11A, ARHGAP33, ARHGAP4, ARHGEF10, ARHGEF3, ARHGEF35, OR2A1-AS1, ARHGEF35, OR2A1-AS1, ARHGEF34P, ARID1B, ARHGEF35, OR2A20P, OR2A1-AS1, ARHGEF9, ARL1, ARL13B, ARL16, ARL6, ARMC6, ARMC8, ARMCX2, ARMCX5, RP4-769N13.6, ARMCX5-GPRASP2, BHLHB9, ARMCX5-GPRASP2, GPRASP1, ARMCX5-GPRASP2, GPRASP2, ARMCX6, ARNT2, ARPP19, ARRB2, ARSA, ART3, ASB3, GPR75-ASB3, ASCC2, ASNS, ASNS, AC079781.5, ASPSCR1, ASS1, ASUN, ATE1, ATF1, ATF7IP2, ATG13, ATG4D, ATG7, ATG9A, ATM, ATOX1, ATP1B3, ATP2C1, ATP5F1A, ATP5G2, ATP5J, ATP5MD, ATP5PF, ATP6AP2, ATP6V0B, ATP6V1C1, ATP6VID, ATP7B, ATXN1, ATXA1L, IST1, ATXA3, ATXA7L1, AURKA, AURKB, AXDND1, B3GALNT1, B3GALT5, AF064860.1, B3GALT5, AF064860.5, B3GNT5, B4GALT3, B4GALT4, B9D1, BACH1, BAIAP2, BANF1, BANF2, BAX, BAZ2A, BBIP1, BCHE, BCL2L14, BCL6, BCL9L, BCS1L, BDH1, BDKRB2, AL355102.2, BEST1, BEST3, BEX4, BHLHB9, BID, BIN3, BIRC2, BIVM, BIVM-ERCC5, BIVM, BLCAP, BLK, BLOC1S1, RP11-644F5.10, BLOC1S6, AC090527.2, BLOC1S6, RP11-96020.4, BLVRA, BMF, BOLA1, BORCS8-MEF2B, BORCS8, BRCA1, BRD1, BRDT, BRINP3, BROX, BTBD10, BTBD3, BTBD9, BTD, BTF3L4, BTNL9, BUB1B-PAK6, PAK6, BUB3, C10orf68, C11orf1, C11orf48, C11orf54, C11orf54, AP001273.2, C11orf57, C11orf63, C11orf82, C12orf23, C12orf4, C12orf65, C12orf79, C14orf159, C14orf93, C17orf62, C18orf21, C19orf12, C19orf40, C19orf47, C19orf48, C19orf54, C1D, C1GALT1, C1QB, C1QTNF1, C1S, C1orf101, C1orf112, C1orf116, C1orf159, C1orf63, C2, C2, CFB, C20orf27, C21orf58, C2CD4D, C2orf15, LIPT1, MRPL30, C2orf80, C2orf81, C3orf14, C3orf17, C3orf18, C3orf22, C3orf33, AC104472.3, C4orf33, C5orf28, C5orf34, C6orf118, C6orf203, C6orf211, C6orf48, C7orf50, C7orf55, C7orf55-LUC7L2, LUC7L2, C8orf44-SGK3, C8orf44, C8orf59, C9, DAB2, C9orf153, C9orf9, CA5BP1, CA5B, CABYR, CALCA, CALCOCO1, CALCOCO2, CALM1, CALM3, CALML4, RP11-315D16.2, CALN, CALU, CANT1, CANX, CAP1, CAPN12, CAPS2, CARD8, CARHSP1, CARNS1, CASC1, CASP3, CASP7, CBFA2T2, CBS, CBY1, CCBL1, CCBL2, RBMXL1, CCDC12, CCDC126, CCDC14, CCDC149, CCDC150, CCDC169-SOHLH2, CCDC169, CCDC171, CCDC37, CCDC41, CCDC57, CCDC63, CCDC7, CCDC74B, CCDC77, CCDC82, CCDC90B, CCDC91, CCDC92, CCNE1, CCHCR1, CCL28, CCNB11P1, CCNC, CCND3, CCNG1, CCP110, CCR9, CCT7, CCT8, CD151, CD1D, CD200, CD22, CD226, CD276, CD36, CD59, CDC26, CDC42, CDC42SE1, CDC42SE2, CDHR3, CDK10, CDK16, CDK4, CDKAL1, CDKL3, CTD-2410N18.4, CDKN1A, CDKN2A, CDNF, CEBPZOS, CELF1, CEMIP, CENPK, CEP170B, CEP250, CEP57, CEP57L1, CEP63, CERS4, CFL1, CFL2, CFLAR, CGNL1, CHCHD7, CHD1L, CHD8, CHFR, ZNF605, CHIA, CHID1, CHL1, CHM, CHMP1A, CHMP3, RNF103-CHMP3, CHRNA2, CIDEC, CIRBP, CITED1, CKLF-CMTM1, CMTM1, CKMT1B, CLDN12, CTB-13L3.1, CLDND1, AC021660.3, CLDND1, CPOX, CLHC1, CLIP1, CLUL1, CMC4, MTCP1, CNDP2, CNFN, CNOT1, CNOT6, CNOT7, CNOT8, CNR1, CNR2, CNTFR, CNTRL, COA1, COASY, COCH, COL8A1, COLCA1, COLEC11, COMMD3-BMI1, BMI1, COPS5, COPS7B, COQ8A, CORO6, COTL1, COX14, RP4-60503.4, COX7A2, COX7A2L, COX7B2, CPA4, CPA5, CPEB1, CPNE1, AL109827.1, RBM12, CPNE1, RP1-309K20.6, RBM12, CPNE3, CPSF3L, CPT1C, CREB3L2, CREM, CRP, CRYZ, CS, AC073896.1, CS, RP11-977G19.10, CSAD, CSDE, CSF2RA, CSGALNACT1, CSK, CSNK2A1, CSRNP2, CT45A4, CT45A4, CT45A5, CT45A6, CTBP2, CTCFL, CTD-2116N17.1, KIAA0101, CTD-2349B8.1, SYT17, CTD-2528L19.4, ZNF607, CTD-2619J13.8, ZNF497, CTNNA1, CTNNBIP1, CTNND1, CTPS2, CTSB, CTSL, CTTN, CUL2, CUL9, CWC15, CXorf40B, CYB561A3, CYBC1, CYLD, CYP11A1, CYP2R1, CYP4B1, CYP4F22, DAG1, DAGLB, KDELR2, DARS, DBNL, DCAF11, DCAF8, PEX19, DCLRE1C, DCTD, DCTN1, DCTN4, DCUN1D2, DDR1, DDX11, DDX19B, AC012184.2, DDX19B, RP11-529K10.3, DDX25, DDX39B, ATP6V1G2-DDX39B, SNORD84, DDX42, DDX60L, DEDD, DEDD2, DEFA1, DEFA1B, DEFA1B, DEFA3, DENND1C, DENND2A, DENND4B, DET, DGKA, DGKZ, DGLUCY, DHRS4L2, DHRS9, DHX40, DIABLO, AC048338.1, DIAPH1, DICER1, DKKL1, DLG1, DLG3, DLST, DMC1, DMKN, DMTF1, DMTN, DNAJC14, DNAJC19, DNAL1, DNASE1L1, DNMT3A, DOC2A, DOCK8, DOK1, DOPEY1, DPAGT1, DPP8, DRAM2, DRD2, DROSHA, DSN1, DTNA, DTX2, DTX3, DUOX1, DUOXA1, DUS2, DUSP10, DUSP13, DUSP18, DUSP22, DYDC1, DYDC2, DYNLL1, DYNLT1, DYRK1A, DYRK2, DYRK4, RPL1-500M8.7, DZIP1L, E2F6, ECHDC1, ECSIT, ECT2, EDC3, EDEM1, EDEM2, MMP24-AS1, RP4-61404.11, EEF1AKNMT, EEF1D, EFEMP1, EFHC1, EGFL7, EHF, EI24, EIF1AD, EIF2B5, EIF4G1, EIF2B5, POLR2H, EIF3E, EIF3K, EIF4E3, EIF4G1, ELF1, ELMO2, ELMOD1, AP000889.3, ELMOD3, ELOC, ELOF1, ELOVL1, ELOVL7, ELP1, ELP6, EML3, EMP3, ENC1, ENDOV, ENO1, ENPP5, ENTHD2, ENTPD6, EP400NL, EPB41L1, EPDR1, NME8, EPHX1, EPM2A, EPN1, EPN2, EPN3, EPS8L2, ERBB3, ERC1, ERCC1, ERG, ERI2, ERI2, DCUN1D3, ERLIN2, ERMARD, ERRFI1, ESR2, RP11-544I20.2, ESRRA, ESRRB, ESRRG, ETFA, ETFRF1, ETV1, ETV4, ETV7, EVA1A, EVC2, EVX1, EXD2, EXO5, EXOC1, EXOC2, FAAP24, FABP6, FADS1, FADS2, FAHD2B, FAM107B, FAM111A, FAM111B, FAM114A1, FAM114A2, FAM115C, FAM115C, FAM115D, FAM120B, FAM133B, FAM135A, FAM153A, FAM153B, FAM154B, FAM156A, FAM156B, FAM168B, FAM172A, FAM182B, FAM192A, FAM19A2, FAM200B, FAM220A, FAM220A, AC009412.1, FAM222B, FAM227B, FAM234A, AC004754.1, FAM3C, FAM45A, FAM49B, FAM60A, FAM63A, FAM81A, FAM86B1, FAM86B2, FANCI, FANK1, FAR2, FAXC, FAXDC2, FBF1, FBH1, FBXL4, FBXO18, FBXO22, FBXO31, FBXO41, FBXO44, FBXO45, FBXW9, FCHO1, FCHSD2, FDFT1, FDPS, FER, FETUB, FGD4, FGF1, FGFR1, FGFRL1, FGL1, FHL2, FIBCD1, FIGNL1, FIGNL1, DDC, FKBP5, FKRP, FLRT2, FLRT3, FMC1, LUC7L2, FMC1-LUC7L2, FNDC3B, FOLH1, FOLR1, FOXP1, FOXK1, FOAM1, FOXO1, FOXP4, AC097634.4, FOXRED1, FPR1, FPR2, FRG1B, FRS2, FTO, FTSJ1, FUK, FUT10, FUT3, FUT6, FXYD3, FZD3, G2E3, GAA, GABARAPL1, GABPB1, GABRA5, GAL3ST1, GALE, GALNT1l, GALNT14, GALNT6, GAPVD1, GARNL3, GAS2L3, GAS8, GATA1, GATA2, GATA4, GBA, GCNT1, GDPD2, GDPD5, GEMIN7, MARK4, GEMIN8, GGA3, GGACT, AL356966.1, GGPS1, GHRL, GID8, GIGYF2, GIMAP8, GIPCI, GJB1, GJB6, GLB1L, GLI1, GLT8D1, GMFG, GMPR2, GNAI2, GNAQ, GNBI, GNB2, GNE, GNG2, GNGT2, GNPDA1, GNPDA2, GOLGA3, CHFR, GOLGA4, GOLPH3L, GOLT1B, GPBP1L1, GPER1, GPR116, GPR141, EPDR1, GPR155, GPR161, GPR56, GPR63, GPR75-ASB3, ASB3, GPR85, GPSM2, GRAMD1B, GRB10, GRB7, GREM2, GRIA2, GSDMB, GSE1, GSN, GSTA4, GSTZ1, GTDC1, GTF2H1, GTF2H4, VARS2, GTF3C2, GUCY1A3, GUCY1B3, GUK1, GULP1, GYPC, GYS1, GZF1, HAGH, HAO2, HAPLN3, HAVCR1, HAX1, HBG2, AC104389.4, HBG2, AC104389.4, HBE1, HBG2, AC104389.4, HBE1, OR51B5, HBG2, HBE1, AC104389.28, HBS1L, HCFC1R1, HCK, HDAC2, HDAC6, HDAC7, HDLBP, HEATR4, HECTD4, HEXIM2, HHAT, HHATL, CCDC13, HINFP, HIRA, C22orf39, HIVEP3, HJV, HKR1, HLF, HMBOX1, HMGA1, HMGB3, HMGCR, HMGN4, HMOX2, HNRNPC, HNRNPD, HNRNPH1, HNRNPH3, HNRNPR, HOMER3, HOPX HOXA3, HOXB3, HOXB3, HOXB4, HOXC4, HOXD3, HOXD3, HOXD4, HPCAL1, HPS4, HPS5, HRH1, HS3ST3A1, HSH2D, HSP90AA1, HSPD1, HTT, HUWE1, HYOU1, IAH1, ICA1L, ICAM2, ICE2, ICK, IDH2, IDH3G, IDS, IFI27, IFI44, IFT20, IFT22, IFT88, IGF2, INS-IGF2, IGF2BP3, IGFBP6, IKBKAP, IKBKB, IL11, IL18BP, IL18RAP, IL1RAP, IL1RL1, IL18R1, IL1RN, IL32, IL4I1, NUP62, AC011452.1, IL4I1, NUP62, CTC-326K19.6, IL6ST, ILVBL, IMALP1L, IMPDH1, INCA1, ING1, INIP, INPP1, INPP5J, INPP5K, INSIG2, INTS11, INTS12, INTS14, IP6K2, IP6K3, IPO11, LRRC70, IQCE, IQGAP3, IRAK4, IRF3, IRF5, IRF6, ISG20, IST1, ISYNA1, ITFG2, ITGB1BP1, ITGB7, ITIH4, RP5-966M1.6, ITPRIPL1, JADE1, JAK2, JARID2, JDP2, KANK1, KANK1, RP11-31F19.1, KANK2, KANSL1L, KAT6A, KBTBD2, KBTBD3, KCNAB2, KCNE3, KCNG1, KCNJ16, KCNJ9, KCNMB2, AC117457.1, LINC01014, KCTD20, KCTD7, RABGEF1, KDM1B, KDM4A, AL451062.3, KHNYN, KIAA0040, KIAA0125, KIAA0196, KIAA0226L, PPP1R2P4, KIAA0391, KIAA0391, AL121594.1, KIAA0391, PSMA6, KIAA0753, KIAA0895, KIAA0895L, KIAA1191, KIAA1407, KIAA1841, C2orf74, KIF12, KIF14, KIF27, KIF9, KIFC3, KIN, KIRREL1, KITLG, KLC1, APOPT1, AL139300.1, KLC4, KLHDC4, KLHDC8A, KLHL13, KLHL18, KLHL2, KLHL24, KLHL7, KLK11, KLK2, KLK5, KLK6, KLK7, KNOP1, KRBA2, AC135178.2, KRBA2, RP11-849F2.7, KRIT1, KRT15, KRT8, KTN1, KXD1, KYAT3, RBMXL1, KYNU, L3MBTL1, LACC1, LARGE, LARP4, LARP7, LAT2, LBHD1, LCA5, LCA5L, LCTL, LEPROTL1, LGALS8, LGALS9C, LGMN, LHFPL2, LIG4, LIMCH1, LIMK2, LIMS2, LINC00921, ZNF263, LIPF, LLGL2, LMAN2L, LMCD1, LMF1, RP11-161M6.2, LMO1, LMO3, LOXHD1, LPAR1, LPAR2, LPAR4, LPAR5, LPAR6, LPHN1, LPIN2, LPIN3, LPP, LRFN5, LRIF1, LRMP, LRRC14, LRRC20, LRRC24, C8orf82, LRRC39, LRRC42, LRRC48, LRRC4C, LRRC8A, LRRC8B, LRRD1, LRTOMT, LRTOMT, AP000812.5, LSM7, LTB4R, LTBP3, LUC7L2, FMC1-LUC7L2, LUC7L3, LUZP1, LYG1, LYL1, LYPD4, LYPD6B, LYRM1, LYRM5, LYSMD4, MACC1, MAD1L1, MAD1L1, AC069288.1, MAEA, MAFF, MAFG, MAFK, MAGEA12, CSAG4, MAGEA2, MAGEA2B, MAGEA4, MAGEB1, MAGOHB, MAN2A2, MANBAL, MAOB, MAP2K3, MAP3K7CL, MAP3K8, MAP7, MAP9, MAPK6, MAPK7, MAPK8, MAPKAP1, 10-Mar, 7-Mar, 8-Mar, MARK2, MASP1, MATK, MATR3, MATR3, SNHG4, MB, MBD5, MBNL1, MBOAT7, MCC, MCFD2, MCM9, MCOLN3, MCRS1, MDC1, MDGA2, MDH2, MDM2, ME1, MEAK7, MECR, MED4, MEF2A, MEF2B, BORCS8-MEF2B, MEF2BNB-MEF2B, MEF2B, MEF2BNB, MEF2C, MEF2D, MEGF10, MEI1, MEIS2, MELK, MET, METTL13, METTL23, MFF, MFN2, MFSD2A, MGST3, MIB2, MICAL1, MICAL3, MICOS10, NBL1, MICOS10-NBLJ, MID1, MINA, MINOS1-NBL1, MINOS1, MIOS, MIPOL1, MIS12, MKLN1, MKNK1, MKNK1, MOB3C, MLF2, MLH1, MMP17, MOBP, MOCS1, MOGS, MOK, MORF4L1, MPC1, MPC2, MPG, MPI, MPP1, MPP2, MPPE1, MPST, MRAS, MRO, MROH1, MROH7-TTC4, MROH7, MRPL14, MRPL24, MRPL33, BABAM2, MRPL33, BRE, MRPL47, MRPL48, MRPL55, MRRF, MRTFA, MRTFB, MRVIJ, MS4A1, MS4A15, MS4A3, MS4A6E, MS4A7, MS4A14, MSANTD3, MSANTD4, MSH5, MSH5-SAPCD1, MSL2, MSRB3, MSS51, MTCP1, CMC4, MTERF, MTERF1, MTERF3, MTERFD2, MTERFD3, MTF2, MTG2, MTHFD2, MTHFD2L, MTIF2, MTIF3, MTMR10, MTRFJ, MTRR, MTUS2, MUTYH, MVK, MX1, MX2, MYH10, MYL12A, MYB, MYD88, MYL5, MYLIP, MYNN, MYO15A, MYO1B, MYOM2, MZF1, N4BP2L2, NAA60, NAB1, NAE1, NAGK, NAP1L1, NAP1L4, NAPG, NARFL, NARG2, NAT1, NAT10, NBPF11, WI2-3658N16.1, NBPF12, NBPF15, NBPF24, NBPF6, NBPF9, NBR1, NCAPG2, NCBP2, NCEH1, NCOA1, NCOA4, NDC1, NDRG1, NDRG2, NDRG4, NDST1, NDUFAF6, NDUFB2, NDUFC1, NDUFS1, NDUFS8, NDUFV1, NEDD1, NEIL1, NEIL2, NEK10, NEK11, NEK6, NEK9, NELFA, NEU4, NFAT5, NFE2, NFE2L2, AC019080.1, NFRKB, NFYA, NFYC, NIF3L1, NIPA2, NKIRAS1, NKX2-1, NLRC3, NME1, NME1-NME2, NME2, NME1-NME2, NME2, NME4, NME6, NME9, NOD1, NOLO, NOL8, NONO, NPAS1, NPIPA8, RP11-1212A22.1, NPIPB3, NPIPB4, NPIPB9, NPL, NPM1, NPPA, NQO2, NR1H3, NR2C2, NR2F2, NR4A1, NRDC, NREP, NRF1, NRG4, NRIP1, NSD2, NSDHL, NSG1, NSMCE2, NSRP1, NT5C2, NTF4, NTMT1, NTNG2, NUBP2, NUCB2, NUDT1, NUDT2, NUDT4, NUF2, NUMBL, NUP50, NUP54, NUP85, NVL, NXF1, NXPE1, NXPE3, OARD1, OAT, OAZ2, OCIAD1, OCLN, ODF2, OGDHL, OGFOD2, AC026362.1, OGFOD2, RP11-197N18.2, OLA1, OPRL1, OPTN, OR2H1, ORAI2, ORMDL1, ORMDL2, ORMDL3, OSBPL2, OSBPL3, OSBPL5, OSBPL9, OSER1, OSGIN1, OSR2, P2RX4, P2RY2, P2RY6, P4HA2, PABPC1, PACRGL, PACSIN3, PADI1, PAIP2, PAK1, PAK3, PAK4, PAK7, PALB2, PANK2, PAQR6, PARPH1, PARVG, PASK, PAX6, PBRM1, PBXIP1, PCBP3, PCBP4, AC115284.1, PCBP4, RP11-155D18.14, RP11-155D18.12, PCGF3, PCGF5, PCNP, PCSK9, PDCD10, PDCD6, AHRR, PDDC1, PDGFRB, PDIA6, PDIK1L, PDLIM7, PDP1, PDPK1, PDPN, PDZD11, PEA15, PEX2, PEX5, PEX5L, PFKM, PFN4, PGAP2, PGAP2, AC090587.2, PGAP3, PGM3, PGPEP1, PHB, PHC2, PHF20, PHF21A, PHF23, PHKB, PHLDB1, PHOSPHO1, PHOSPHO2, KLHL23, PI4 KB, PIAS2, PICALM, PIF1, PIGN, PIGO, PIGT, PIK3CD, PILRB, STAG3L5P-PVRIG2P-PILRB, PIP5K1B, PIR, PISD, PIWIL4, FUT4, PKD2, PKIA, PKIG, PKM, PKN2, PLA1A, PLA2G2A, PLA2G5, PLA2G7, PLAC8, PLAGL1, PLD1, PLD3, PLEKHA1, PLEKHA2, PLEKHA6, PLEKHG5, PLIN1, PLS1, PLS3, PLSCR1, PLSCR2, PLSCR4, PLXNB1, PLXNB2, PMP22, PMS1, PNISR, PNKP, AKT1S1, PNMT, PNPLA4, PNPLA8, PNPO, PNRCI, POCIB, POFUT1, POLB, POLD1, POLH, POLI, POLL, POLR1B, POM121, POM121C, AC006014.7, POM121C, AC211429.1, POMC, POMT1, POP1, PORCN, POU5F1, PSORSIC3, PPARD, PPARG, PPHLN, PPIL3, PPIL4, PPM1A, PPM1B, AC013717.1, PPP1CB, PPP1R11, PPPIR13L, PPP1R26, PPP1R9A, PPP2R2B, PPP3CA, PPP6R1, PPP6R3, PPT2, PPT2-EGFL8, EGFL8, PPWD1, PRDM2, PRDM8, PRELID3A, PREPL, PRICKLE1, PRKAG1, PRMT2, PRMT5, PRMT7, PROM1, PRPS1, PRPSAP2, PRR14L, PRR15L, PRR5, PRR5-ARHGAP8, PRR5L, PRR7, PRRC2B, PRRT4, PRSS50, PRSS45, PRSS44, PRUNE, PRUNE1, PSEN1, PSMA2, PSMF1, PSORS1C1, PSPH, PSRC1, PTBP3, PTHLH, PTK2, PTPDC1, PTPRM, PUF60, PUM2, PUS1, PUS10, PXA, PXYLP1, PYCR1, QRICH1, R3HCC1L, R3HDM2, RAB17, RAB23, RAB3A, RAB3D, TMEM205, RAB4B-EGLN2, EGLN2, AC008537.1, RAB5B, RAB7L1, RABL2A, RABL2B, RABL5, RACGAP1, RAD17, RAD51L3-RFFL, RAD51D, RAD52, RAE1, RAI14, RAI2, RALBP1, RAN, RANGAP1, RAP1A, RAP1B, RAP1GAP, RAPGEF4, RAPGEFL1, RASGRP2, RASSF1, RBCK1, RBM12B, RBM14, RBM4, RBM14-RBM4, RBM23, RBM4, RBM14-RBM4, RBM47, RBM7, AP002373.1, RBM7, RP11-212D19.4, RBMS2, RBMY1E, RBPJ, RBPMS, RBSN, RCBTB2, RCC1, RCC1, SNHG3, RCCD1, RECQL, RELL2, REPIN1, AC073111.3, REPIN1, ZNF775, RER1, RERE, RFWD3, RFX3, RGL2, RGMB, RGS11, RGS3, RGS5, AL592435.1, RHBDD1, RHNO1, TULP3, RHOC, AL603832.3, RHOC, RP11-426L16.10, RHOH, RIC8B, RIMKLB, RIN1, RIPK2, RIT1, RLIM, RNASE4, ANG, AL163636.6, RNASEK, RNASEK-C17orf49, RNFI1, RNF123, RNF13, RNF14, RNF185, RNF216, RNF24, RNF32, RNF34, RNF38, RNF4, RNF44, RNH1, RNMT, RNPS1, RO60, ROPN1, ROPN1B, ROR2, RP1-102H19.8, C6orf163, RP1-283E3.8, CDK11A, RP11-120M18.2, PRKAR1A, RP11-133K10.2, PAK6, RP11-164J13.1, CAPN3, RP11-21J18.1, ANKRD12, RP11-322E10.6, INO80C, RP11-337C18.10, CHD1L, RP11-432B6.3, TRIM59, RP11-468E2.4, IRF9, RP11-484M3.5, UPK1B, RP11-517H2.6, CCR6, RP11-613M10.9, SLC25A51, RP11-659G9.3, RAB30, RP11-691N7.6, CTNND1, RP11-849H4.2, RP11-896J10.3, NKX2-1, RP11-96020.4, SQRDL, RP11-986E7.7, SERPINA3, RP4-769N13.6, GPRASP1, RP4-769N13.6, GPRASP2, RP4-798P15.3, SEC16B, RP5-10211I20.4, ZNF410, RP6-109B7.3, FLJ27365, RPE, RPH3AL, RPL15, RPL17, RPL17-C18orf32, RPL17, RPL23A, RPL36, HSD11B1L, RPP38, RPS20, RPS27A, RPS3A, RPS6KA3, RPS6KC1, RPS6KL1, RPUSD1, RRAGD, RRAS2, RRBP1, RSL1D1, RSRC2, RSRP1, RUBCNL, RUNX1T1, RUVBL2, RWDDJ, RWDD4, S100A13, AL162258.1, S100A13, RP1-178F15.5, S100A16, S100A4, S100A3, S100A6, S100PBP, SAA1, SACM1L, SAMD4B, SAR1A, SARAF, SARNP, RP11-762I7.5, SCAMP5, SCAP, SCAPER, SCFD1, SCGB3A2, SCIN, SCML1, SCNN1D, SCO2, SCOC, SCRN1, SDC2, SDC4, SEC13, SEC14L1, SEC14L2, SEC22C, SEC23B, SEC24C, SEC61G, SEMA4A, SEMA4C, SEMA4D, SEMA6C, SENP7, SEPP1, 11-Sep, 2-Sep, SERGEF, AC055860.1, SERP1, SERPINA1, SERPINA5, SERPINB6, SERPING1, SERPINH1, SERTAD3, SETD5, SFMBT1, AC096887.1, SFTPA1, SFTPA2, SFXN2, SGCD, SGCE, SGK3, SGK3, C8orf44, SH2B1, SH2D6, SH3BP1, Z83844.3, SH3BP2, SH3BP5, SH3D19, SH3YL1, SHC1, SHISA5, SHMT1, SHMT2, SHOC2, SHROOM1, SIGLEC5, SIGLEC14, SIL1, SIN3A, SIRT2, SIRT6, SKP1, STAT4, AC104109.3, SLAIN1, SLC10A3, SLC12A9, SLC14A1, SLC16A6, SLC1A2, SLC1A6, SLC20A2, SLC25A18, SLC25A19, SLC25A22, SLC25A25, SLC25A29, SLC25A30, SLC25A32, SLC25A39, SLC25A44, SLC25A45, SLC25A53, SLC26A11, SLC26A4, SLC28A1, SLC29A1, SLC2A14, SLC2A5, SLC2A8, SLC35B2, SLC35B3, SLC35C2, SLC37A1, SLC38A1, SLC38A11, SLC39A13, SLC39A14, SLC41A3, SLC44A3, SLC4A7, SLC4A8, SLC5A10, SLC5A11, SLC6A1, SLC6A12, SLC6A9, SLC7A2, SLC7A6, SLC7A7, SLCO1A2, SLCO1C1, SLCO2B1, SLFN11, SLFN12, SLFNL1, SLMO1, SLTM, SLU7, SMAD2, SMAP2, SMARCA2, SMARCE1, AC073508.2, SMARCE1, KRT222, SMC6, SMG7, SMIM22, SMOX, SMPDL3A, SMTN, SMU1, SMUG1, SNAP25, SNCA, SNRK, SNRPC, SNRPD1, SNRPD2, SNRPN, SNRPN, SNURF, SNUPN, SNX11, SNX16, SNX17, SOAT1, SOHLH2, CCDC169-SOHLH2, CCDC169, SORBS1, SORBS2, SOX5, SP2, SPART, SPATA20, SPATA21, SPATS2, SPATS2L, SPDYE2, SPECC1, SPECC1L, SPECC1L-ADORA2A, SPECC1L-ADORA2A, ADORA2A, SPEG, SPG20, SPG21, SPIDR, SPIN1, SPOCD1, SPOP, SPRR2A, SPRR2B, SPRR2E, SPRR2B, SPRR2F, SPRR2D, SPRR3, SPRY1, SPRY4, SPTBN2, SRC, SRGAP1, SRP68, SRSF11, SSX1, SSX2IP, ST3GAL4, ST3GAL6, ST5, ST6GALNAC6, ST7L, STAC3, STAG1, STAG2, STAMBP, STAMBPL1, STARD3NL, STAT6, STAU1, STAU2, AC022826.2, STAU2, RP11-463D19.2, STEAP2, STEAP3, STIL, STK25, STK33, STK38L, STK40, STMN1, STON1, STON1-GTF2AIL, STRAP, STRBP, STRC, AC011330.5, STRC, CATSPER2, STRC, CATSPER2, AC011330.5, STRC, STRCP1, STT3A, STX16-NPEPL1, NPEPL1, STX5, STX6, STX8, STXBP6, STYK1, SULT1A1, SULT1A2, SUMF2, SUN1, SUN2, SUN2, DNAL4, SUOX, SUPT6H, SUV39H2, SV2B, SYBU, SYNCRIP, SYNJ2, SYT1, SYTL4, TAB2, TACC1, TADA2B, TAF1C, TAF6, AC073842.2, TAF6, RP11-506M12.1, TAF9, TAGLN, TANK, TAPSAR1, PSMB9, TAPT1, TATDN1, TAZ, TBC1D1, TBC1D12, HELLS, TBC1D15, TBCID3H, TBC1D3G, TBCID5, TBCID5, SATB1, TBCA, TBCEL, TBCEL, AP000646.1, TBL1XR1, TBP, TBX5, TBXAS1, TCAF1, TCEA2, TCEAL4, TCEAL8, TCEAL9, TCEANC, TCEB1, TCF19, TCF25, TCF4, TCP1, TCP10L, AP000275.65, TCP11, TCP11L2, TCTN1, TDG, TDP1, TDRD7, TEAD2, TECR, TENC1, TENT4A, TEX264, TEX30, TEX37, TFDP1, TFDP2, TFEB, TFG, TFP1, TF, TFP1, TGIF1, THAP6, THBS3, THOC5, THRAP3, THUMPD3, TIAL1, TIMM9, TIMP1, TIRAP, TJAP1, TJP2, TK2, TLDC1, TLE3, TLE6, TLN1, TLR10, TM9SF1, TMBIM1, TMBIM4, TMBIM6, TMC6, TMCC1, TMCO4, TMEM126A, TMEM139, TMEM150B, TMEM155, TMEM161B, TMEM164, TMEM168, TMEM169, TMEM175, TMEM176B, TMEM182, TMEM199, CTB-96E2.3, TMEM216, TMEM218, TMEM230, TMEM263, TMEM45A, TMEM45B, TMEM62, TMEM63B, TMEM66, TMEM68, TMEM98, TMEM9B, TMPRSS11D, TMPRSS5, TMSB15B, TMTC4, TMUB2, TMX2-CTNND1, RP11-691N7.6, CTNND1, TNFAIP2, TNFAIP8L2, SCNM1, TNFRSF10C, TNFRSF9, TNFRSF8, TNFSF12-TNFSF3, TNFSF2, TNFSF3, TNFSF12-TNFSF3, TNFSF3, TNIP1, TNK2, TNNT, TNRC18, TNS3, TOB2, TOM1L1, TOP1MT, TOP3B, TOX2, TP53, RP11-199F1.2, TP53I1, TP53INP2, TPCN, TPM3P9, AC022137.3, TPT1, TRA2B, TRAF2, TRAF3, TRAPPC12, TRAPPC3, TREH, TREX, TREX2, TRIB2, TRIM3, TRIM36, TRIM39, TRIM46, TRIM6, TRIM6-TRIM34, TRIM6-TRIM34, TRIM34, TRIM66, TRIM73, TRIT1, TRMT10B, TRMT2B, TRMT2B-AS1, TRNT1, TRO, TROVE2, TRPS1, TRPT1, TSC2, TSGA10, TSPAN14, TSPAN3, TSPAN4, TSPAN5, TSPAN6, TSPAN9, TSPO, TTC12, TTC23, TTC3, TTC39A, TTC39C, TTLL1, TTLL7, TTPAL, TUBD1, TWNK, TXNL4A, TXNL4B, TXNRD1, TYK2, U2AF1, UBA2, UBA52, UBAP2, UBE2D2, UBE2D3, UBE2E3, UBE2I, UBE2J2, UBE3A, UBL7, UBXN11, UBXN7, UGDH, UGGT1, UGP2, UMAD1, AC007161.3, UNC45A, UQCC1, URGCP-MRPS24, URGCP, USMG5, USP16, USP21, USP28, USP3, USP33, USP35, USP54, USP9Y, USPL1, UTP15, VARS2, VASH2, VAV3, VDAC1, VDAC2, VDR, VEZT, VGF, VIL1, VILL, VIPR1, VPS29, VPS37C, VPS8, VPS9D1, VRK2, VWA1, VWA5A, WARS, WASF1, WASHC5, WBP5, WDHD1, WDPCP, WDR37, WDR53, WDR6, WDR72, WDR74, WDR81, WDR86, WDYHV1, WFDC3, WHSC1, WIPF1, WSCD2, WWP2, XAGE1A, XAGEIB, XKR9, XPNPEP1, XRCC3, XRN2, XXYLT1, YIF1A, YIF1B, YIPF1, YIPF5, YPEL5, YWHAB, YWHAZ, YY1AP1, ZBTB1, ZBTB14, ZBTB18, ZBTB20, ZBTB21, ZBTB25, ZBTB33, ZBTB34, ZBTB38, ZBTB43, ZBTB49, ZBTB7B, ZBTB7C, ZBTB8OS, ZC3H11A, ZBED6, ZC3H13, ZCCHC17, ZCCHC7, ZDHHC11, ZDHHC13, ZEB2, ZFAND5, ZFAND6, ZFP1, ZFP62, ZFX, ZFYVE16, ZFYVE19, ZFYVE20, ZFYVE27, ZHX2, AC016405.1, ZHX3, ZIK1, ZIM2, PEG3, ZKSCAN1, ZKSCAN3, ZKSCAN8, ZMAT3, ZMAT5, ZMIZ2, ZMYM6, ZMYND11, ZNF10, AC026786.1, ZNF133, ZNF146, ZNF16, ZNF177, ZNF18, ZNF200, ZNF202, ZNF211, ZNF219, ZNF226, ZNF227, ZNF23, AC010547.4, ZNF23, AC010547.9, ZNF239, ZNF248, ZNF25, ZNF253, ZNF254, ZNF254, AC092279.1, ZNF263, ZNF274, ZNF275, ZNF28, ZNF468, ZNF283, ZNF287, ZNF3, ZNF320, ZNF322, ZNF324B, ZNF331, ZNF334, ZNF34, ZNF350, ZNF385A, ZNF395, FBXO16, ZNF415, ZNF418, ZNF43, ZNF433-AS1, AC008770.4, ZNF438, ZNF444, ZNF445, ZNF467, ZNF480, ZNF493, ZNF493, CTD-2561J22.3, ZNF502, ZNF507, ZNF512, AC074091.1, ZNF512, RP11-158I13.2, ZNF512B, ZNF512B, SAMD10, ZNF521, ZNF532, ZNF544, AC020915.5, ZNF544, CTD-3138B18.4, ZNF559, ZNF177, ZNF562, ZNF567, ZNF569, ZNF570, ZNF571-AS1, ZNF540, ZNF577, ZNF580, ZNF581, ZNF580, ZNF581, CCDC106, ZNF600, ZNF611, ZNF613, ZNF615, ZNF619, ZNF620, ZNF639, ZNF652, ZNF665, ZNF667, ZNF668, ZNF671, ZNF682, ZNF687, ZNF691, ZNF696, ZNF701, ZNF706, ZNF707, ZNF714, ZNF717, ZNF718, ZNF720, ZNF721, ZNF730, ZNF763, ZNF780B, AC005614.5, ZNF782, ZNF786, ZNF79, ZNF791, ZNF81, ZNF83, ZNF837, ZNF839, ZNF84, ZNF845, ZNF846, ZNF865, ZNF91, ZNF92, ZNHIT3, ZSCAN21, ZSCAN25, ZSCAN30, and ZSCAN32.

In some embodiments, the gene encoding a target sequence comprises the HTT gene.

Exemplary genes that may be modulated by the compounds of Formula (I) described herein may also include, inter alia, AC005258.1, AC005943.1, AC007849.1, AC008770.2, AC010487.3, AC011477.4, AC012651.1, AC012531.3, AC034102.2, AC073896.4, AC104472.3, AL109811.3, AL133342.1, AL137782.1, AL157871.5, AF241726.2, AL355336.1, AL358113.1, AL360181.3, AL445423.2, AL691482.3, AP001267.5, RF01169, and RF02271.

The compounds described herein may further be used to modulate a sequence comprising a particular splice site sequence, e.g., an RNA sequence (e.g., a pre-mRNA sequence). In some embodiments, the splice site sequence comprises a 5′ splice site sequence. In some embodiments, the splice site sequence comprises a 3′ splice site sequence. Exemplary gene sequences and splice site sequences (e.g., 5′ splice site sequences) include AAAgcaaguu, AAAguaaaaa, AAAguaaaau, AAAguaaagu, AAAguaaaua, AAAguaaaug, AAAguaaauu, AAAguaacac, AAAguaacca, AAAguaacuu, AAAguaagaa, AAAguaagac, AAAguaagag, AAAguaagau, AAAguaagca, AAAguaagcc, AAAguaagcu, AAAguaagga, AAAguaaggg, AAAguaaggu, AAAguaagua, AAAguaaguc, AAAguaagug, AAAguaaguu, AAAguaaucu, AAAguaauua, AAAguacaaa, AAAguaccgg, AAAguacuag, AAAguacugg, AAAguacuuc, AAAguacuug, AAAguagcuu, AAAguaggag, AAAguaggau, AAAguagggg, AAAguaggua, AAAguaguaa, AAAguauauu, AAAguauccu, AAAguaucuc, AAAguaugga, AAAguaugua, AAAguaugug, AAAguauguu, AAAguauugg, AAAguauuuu, AAAgucagau, AAAgucugag, AAAgugaaua, AAAgugagaa, AAAgugagac, AAAgugagag, AAAgugagau, AAAgugagca, AAAgugagcu, AAAgugaggg, AAAgugagua, AAAgugaguc, AAAgugagug, AAAgugaguu, AAAgugcguc, AAAgugcuga, AAAguggguc, AAAguggguu, AAAgugguaa, AAAguguaug, AAAgugugug, AAAguguguu, AAAguuaagu, AAAguuacuu, AAAguuagug, AAAguuaugu, AAAguugagu, AAAguuugua, AACguaaaac, AACguaaagc, AACguaaagg, AACguaagca, AACguaaggg, AACguaaguc, AACguaagug, AACguaaugg, AACguaguga, AACguaugua, AACguauguu, AACgugagca, AACgugagga, AACgugauuu, AACgugggau, AACgugggua, AACguguguu, AACguuggua, AAGgcaaauu, AAGgcaagag, AAGgcaagau, AAGgcaagcc, AAGgcaagga, AAGgcaaggg, AAGgcaagug, AAGgcaaguu, AAGgcacugc, AAGgcagaaa, AAGgcaggau, AAGgcaggca, AAGgcaggga, AAGgcagggg, AAGgcaggua, AAGgcaggug, AAGgcaucuc, AAGgcaugcu, AAGgcaugga, AAGgcauguu, AAGgcauuau, AAGgcgagcu, AAGgcgaguc, AAGgcgaguu, AAGgcuagcc, AAGguaaaaa, AAGguaaaac, AAGguaaaag, AAGguaaaau, AAGguaaaca, AAGguaaacc, AAGguaaacu, AAGguaaaga, AAGguaaagc, AAGguaaagg, AAGguaaagu, AAGguaaaua, AAGguaaauc, AAGguaaaug, AAGguaaauu, AAGguaacaa, AAGguaacau, AAGguaaccc, AAGguaacua, AAGguaacuc, AAGguaacug, AAGguaacuu, AAGguaagaa, AAGguaagac, AAGguaagag, AAGguaagau, AAGguaagca, AAGguaagcc, AAGguaagcg, AAGguaagcu, AAGguaagga, AAGguaaggc, AAGguaaggg, AAGguaaggu, AAGguaagua, AAGguaaguc, AAGguaagug, AAGguaaguu, AAGguaauaa, AAGguaauac, AAGguaauag, AAGguaauau, AAGguaauca, AAGguaaucc, AAGguaaucu, AAGguaauga, AAGguaaugc, AAGguaaugg, AAGguaaugu, AAGguaauua, AAGguaauuc, AAGguaauug, AAGguaauuu, AAGguacaaa, AAGguacaag, AAGguacaau, AAGguacacc, AAGguacacu, AAGguacagg, AAGguacagu, AAGguacaua, AAGguacaug, AAGguacauu, AAGguaccaa, AAGguaccag, AAGguaccca, AAGguacccu, AAGguaccuc, AAGguaccug, AAGguaccuu, AAGguacgaa, AAGguacggg, AAGguacggu, AAGguacguc, AAGguacguu, AAGguacuaa, AAGguacuau, AAGguacucu, AAGguacuga, AAGguacugc, AAGguacugu, AAGguacuuc, AAGguacuug, AAGguacuuu, AAGguagaaa, AAGguagaac, AAGguagaca, AAGguagacc, AAGguagacu, AAGguagagu, AAGguagaua, AAGguagcaa, AAGguagcag, AAGguagcca, AAGguagccu, AAGguagcua, AAGguagcug, AAGguagcuu, AAGguaggaa, AAGguaggag, AAGguaggau, AAGguaggca, AAGguaggcc, AAGguaggcu, AAGguaggga, AAGguagggc, AAGguagggg, AAGguagggu, AAGguaggua, AAGguagguc, AAGguaggug, AAGguagguu, AAGguaguaa, AAGguaguag, AAGguagucu, AAGguagugc, AAGguagugg, AAGguaguuc, AAGguaguuu, AAGguauaaa, AAGguauaau, AAGguauaca, AAGguauacu, AAGguauaua, AAGguauauc, AAGguauaug, AAGguauauu, AAGguaucac, AAGguaucag, AAGguauccc, AAGguauccu, AAGguaucuc, AAGguaucug, AAGguaucuu, AAGguaugaa, AAGguaugac, AAGguaugag, AAGguaugau, AAGguaugca, AAGguaugcc, AAGguaugcu, AAGguaugga, AAGguauggc, AAGguauggg, AAGguaugua, AAGguauguc, AAGguaugug, AAGguauguu, AAGguauuaa, AAGguauuac, AAGguauuag, AAGguauuau, AAGguauucc, AAGguauuga, AAGguauugu, AAGguauuua, AAGguauuuc, AAGguauuug, AAGguauuuu, AAGgucaaau, AAGgucaaga, AAGgucaagu, AAGgucacag, AAGgucagaa, AAGgucagac, AAGgucagag, AAGgucagca, AAGgucagcc, AAGgucagcg, AAGgucagcu, AAGgucagga, AAGgucaggc, AAGgucaggg, AAGgucaggu, AAGgucagua, AAGgucaguc, AAGgucagug, AAGgucaguu, AAGgucauag, AAGgucaucu, AAGguccaca, AAGguccaga, AAGguccaua, AAGgucccag, AAGgucccuc, AAGguccuuc, AAGgucgagg, AAGgucuaau, AAGgucuacc, AAGgucuaua, AAGgucuccu, AAGgucucug, AAGgucucuu, AAGgucugaa, AAGgucugag, AAGgucugga, AAGgucuggg, AAGgucugua, AAGgucuguu, AAGgucuucu, AAGgucuuuu, AAGgugaaac, AAGgugaaag, AAGgugaaau, AAGgugaacu, AAGgugaagc, AAGgugaagg, AAGgugaagu, AAGgugaaua, AAGgugaaug, AAGgugaauu, AAGgugacaa, AAGgugacag, AAGgugacau, AAGgugacug, AAGgugacuu, AAGgugagaa, AAGgugagac, AAGgugagag, AAGgugagau, AAGgugagca, AAGgugagcc, AAGgugagcg, AAGgugagcu, AAGgugagga, AAGgugaggc, AAGgugaggg, AAGgugaggu, AAGgugagua, AAGgugaguc, AAGgugagug, AAGgugaguu, AAGgugauaa, AAGgugauca, AAGgugaucc, AAGgugauga, AAGgugaugc, AAGgugaugu, AAGgugauua, AAGgugauug, AAGgugauuu, AAGgugcaca, AAGgugcauc, AAGgugcccu, AAGgugccug, AAGgugcgug, AAGgugcguu, AAGgugcucc, AAGgugcuga, AAGgugcugc, AAGgugcugg, AAGgugcuua, AAGgugcuuu, AAGguggaua, AAGguggcua, AAGguggcug, AAGguggcuu, AAGgugggaa, AAGgugggag, AAGgugggau, AAGgugggca, AAGgugggcc, AAGgugggcg, AAGgugggga, AAGguggggu, AAGgugggua, AAGgugggug, AAGguggguu, AAGgugguaa, AAGgugguac, AAGgugguau, AAGguggugg, AAGgugguua, AAGgugguuc, AAGgugguuu, AAGguguaag, AAGgugucaa, AAGgugucag, AAGgugucug, AAGgugugaa, AAGgugugag, AAGgugugca, AAGgugugga, AAGguguggu, AAGgugugua, AAGguguguc, AAGgugugug, AAGguguguu, AAGguguucu, AAGguguugc, AAGguguugg, AAGguguuug, AAGguuaaaa, AAGguuaaca, AAGguuaagc, AAGguuaauu, AAGguuacau, AAGguuagaa, AAGguuagau, AAGguuagca, AAGguuagcc, AAGguuagga, AAGguuaggc, AAGguuagua, AAGguuaguc, AAGguuagug, AAGguuaguu, AAGguuauag, AAGguuauga, AAGguucaaa, AAGguucaag, AAGguuccuu, AAGguucggc, AAGguucguu, AAGguucuaa, AAGguucuga, AAGguucuua, AAGguugaau, AAGguugacu, AAGguugagg, AAGguugagu, AAGguugaua, AAGguugcac, AAGguugcug, AAGguuggaa, AAGguuggca, AAGguuggga, AAGguugggg, AAGguuggua, AAGguugguc, AAGguuggug, AAGguugguu, AAGguuguaa, AAGguugucc, AAGguugugc, AAGguuguua, AAGguuuacc, AAGguuuaua, AAGguuuauu, AAGguuuccu, AAGguuucgu, AAGguuugag, AAGguuugca, AAGguuugcc, AAGguuugcu, AAGguuugga, AAGguuuggu, AAGguuugua, AAGguuuguc, AAGguuugug, AAGguuuuaa, AAGguuuuca, AAGguuuucg, AAGguuuugc, AAGguuuugu, AAGguuuuuu, AAUgcaagua, AAUgcaaguc, AAUguaaaca, AAUguaaaua, AAUguaaauc, AAUguaaaug, AAUguaaauu, AAUguaacua, AAUguaagaa, AAUguaagag, AAUguaagau, AAUguaagcc, AAUguaagcu, AAUguaagga, AAUguaagua, AAUguaaguc, AAUguaagug, AAUguaaguu, AAUguaauca, AAUguaauga, AAUguaaugu, AAUguacauc, AAUguacaug, AAUguacgau, AAUguacgua, AAUguacguc, AAUguacgug, AAUguacucu, AAUguaggca, AAUguagguu, AAUguaucua, AAUguaugaa, AAUguaugua, AAUguaugug, AAUguauguu, AAUgucagag, AAUgucagau, AAUgucagcu, AAUgucagua, AAUgucaguc, AAUgucagug, AAUgucaguu, AAUgucggua, AAUgucuguu, AAUgugagaa, AAUgugagca, AAUgugagcc, AAUgugagga, AAUgugagua, AAUgugaguc, AAUgugagug, AAUgugaguu, AAUgugauau, AAUgugcaua, AAUgugcgua, AAUgugcguc, AAUgugggac, AAUguggguc, AAUgugggug, AAUgugguuu, AAUgugugua, AAUguuaagu, AAUguuagaa, AAUguuagau, AAUguuagua, AAUguuggug, ACAgcaagua, ACAguaaaua, ACAguaaaug, ACAguaagaa, ACAguaagca, ACAguaagua, ACAguaaguc, ACAguaagug, ACAguaaguu, ACAguacgua, ACAguaggug, ACAguauaac, ACAguaugua, ACAgucaguu, ACAgugagaa, ACAgugagcc, ACAgugagcu, ACAgugagga, ACAgugaggu, ACAgugagua, ACAgugaguc, ACAgugagug, ACAgugaguu, ACAgugggua, ACAguggguu, ACAguguaaa, ACAguuaagc, ACAguuaagu, ACAguuaugu, ACAguugagu, ACAguuguga, ACCguaagua, ACCgugagaa, ACCgugagca, ACCgugaguu, ACCgugggug, ACGguaaaac, ACGguaacua, ACGguaagua, ACGguaagug, ACGguaaguu, ACGguaauua, ACGguaauuu, ACGguacaau, ACGguacagu, ACGguaccag, ACGguacggu, ACGguacgua, ACGguaggaa, ACGguaggag, ACGguaggug, ACGguaguaa, ACGguauaau, ACGguaugac, ACGguaugcg, ACGguaugua, ACGguauguc, ACGgugaaac, ACGgugaagu, ACGgugaauc, ACGgugacag, ACGgugacca, ACGgugagaa, ACGgugagau, ACGgugagcc, ACGgugagua, ACGgugagug, ACGgugaguu, ACGgugcgug, ACGguggcac, ACGguggggc, ACGgugggug, ACGguguagu, ACGgugucac, ACGgugugua, ACGguguguu, ACGguuagug, ACGguuaguu, ACGguucaau, ACUguaaaua, ACUguaagaa, ACUguaagac, ACUguaagca, ACUguaagcu, ACUguaagua, ACUguaaguc, ACUguaaguu, ACUguacguu, ACUguacuge, ACUguaggcu, ACUguaggua, ACUguauauu, ACUguaugaa, ACUguaugcu, ACUguaugug, ACUguauucc, ACUgucagcu, ACUgucagug, ACUgugaacg, ACUgugagca, ACUgugagcg, ACUgugagcu, ACUgugagua, ACUgugaguc, ACUgugagug, ACUgugaguu, ACUgugggua, ACUgugugug, ACUguuaagu, AGAgcaagua, AGAguaaaac, AGAguaaacg, AGAguaaaga, AGAguaaagu, AGAguaaauc, AGAguaaaug, AGAguaacau, AGAguaacua, AGAguaagaa, AGAguaagac, AGAguaagag, AGAguaagau, AGAguaagca, AGAguaagcu, AGAguaagga, AGAguaaggc, AGAguaaggg, AGAguaaggu, AGAguaaguc, AGAguaagug, AGAguaaguu, AGAguaauaa, AGAguaaugu, AGAguaauuc, AGAguaauuu, AGAguacacc, AGAguaccug, AGAguacgug, AGAguacucu, AGAguacuga, AGAguacuuu, AGAguagcug, AGAguaggaa, AGAguaggga, AGAguagggu, AGAguagguc, AGAguaggug, AGAguagguu, AGAguauaua, AGAguauauu, AGAguaugaa, AGAguaugac, AGAguaugau, AGAguauguc, AGAguaugug, AGAguauguu, AGAguauuaa, AGAguauuau, AGAgucagug, AGAgugagac, AGAgugagag, AGAgugagau, AGAgugagca, AGAgugagua, AGAgugaguc, AGAgugagug, AGAgugaguu, AGAgugcguc, AGAgugggga, AGAgugggug, AGAgugugug, AGAguguuuc, AGAguuagua, AGAguugaga, AGAguugagu, AGAguugguu, AGAguuugau, AGCguaagcu, AGCguaagug, AGCgugagcc, AGCgugagug, AGCguuguuc, AGGgcagagu, AGGgcagccu, AGGgcuagua, AGGguaaaga, AGGguaaaua, AGGguaaauc, AGGguaaauu, AGGguaacca, AGGguaacug, AGGguaacuu, AGGguaagaa, AGGguaagag, AGGguaagau, AGGguaagca, AGGguaagga, AGGguaaggc, AGGguaaggg, AGGguaagua, AGGguaaguc, AGGguaagug, AGGguaaguu, AGGguaauac, AGGguaauga, AGGguaauua, AGGguaauuu, AGGguacacc, AGGguacagu, AGGguacggu, AGGguaggac, AGGguaggag, AGGguaggca, AGGguaggcc, AGGguaggga, AGGguagggu, AGGguagguc, AGGguaggug, AGGguagguu, AGGguauaua, AGGguaugac, AGGguaugag, AGGguaugau, AGGguaugca, AGGguaugcu, AGGguauggg, AGGguauggu, AGGguaugua, AGGguauguc, AGGguaugug, AGGguauuac, AGGguauucu, AGGguauuuc, AGGgucagag, AGGgucagca, AGGgucagga, AGGgucaggg, AGGgucagug, AGGgucaguu, AGGguccccu, AGGgucggga, AGGgucugca, AGGgucuguu, AGGgugaaga, AGGgugacua, AGGgugagaa, AGGgugagac, AGGgugagag, AGGgugagca, AGGgugagcc, AGGgugagcu, AGGgugagga, AGGgugaggg, AGGgugaggu, AGGgugagua, AGGgugaguc, AGGgugagug, AGGgugaguu, AGGgugggga, AGGguggggu, AGGgugggua, AGGgugggug, AGGgugugua, AGGgugugug, AGGguuaaug, AGGguuagaa, AGGguuaguu, AGGguuggug, AGGguuugug, AGGguuuguu, AGUguaaaag, AGUguaaaua, AGUguaaauu, AGUguaagaa, AGUguaagag, AGUguaagau, AGUguaagca, AGUguaagcc, AGUguaagua, AGUguaagug, AGUguaaguu, AGUguaauug, AGUguaggac, AGUguagguc, AGUguaugag, AGUguaugua, AGUguauguu, AGUguauugu, AGUguauuua, AGUgucaguc, AGUgugagag, AGUgugagca, AGUgugagcc, AGUgugagcu, AGUgugagua, AGUgugaguc, AGUgugagug, AGUgugaguu, AGUgugggua, AGUgugggug, AGUgugugua, AGUguuccua, AGUguugggg, AGUguuucag, AUAguaaaua, AUAguaagac, AUAguaagau, AUAguaagca, AUAguaagua, AUAguaagug, AUAguaaguu, AUAguaggua, AUAguauguu, AUAgucucac, AUAgugagac, AUAgugagag, AUAgugagau, AUAgugagcc, AUAgugaggc, AUAgugagua, AUAgugaguc, AUAgugagug, AUAgugcguc, AUAgugugua, AUAguucagu, AUCguaagcc, AUCguaaguu, AUCguauucc, AUCgugagua, AUGgcaagcg, AUGgcaagga, AUGgcaaguu, AUGgcaggua, AUGgcaugug, AUGgcgccau, AUGgcuugug, AUGguaaaac, AUGguaaaau, AUGguaaacc, AUGguaaaga, AUGguaaaua, AUGguaaaug, AUGguaaauu, AUGguaacag, AUGguaacau, AUGguaacua, AUGguaacuc, AUGguaacuu, AUGguaagaa, AUGguaagac, AUGguaagag, AUGguaagau, AUGguaagca, AUGguaagcc, AUGguaagcu, AUGguaagga, AUGguaaggg, AUGguaagua, AUGguaaguc, AUGguaagug, AUGguaaguu, AUGguaauaa, AUGguaauau, AUGguaauga, AUGguaaugg, AUGguaauug, AUGguaauuu, AUGguacage, AUGguacauc, AUGguaccag, AUGguaccug, AUGguacgag, AUGguacggu, AUGguagauc, AUGguagcag, AUGguagcug, AUGguaggaa, AUGguaggau, AUGguaggca, AUGguaggcu, AUGguagggg, AUGguagggu, AUGguaggua, AUGguaggug, AUGguaguuu, AUGguauagu, AUGguauaua, AUGguaucag, AUGguaucuu, AUGguaugau, AUGguaugca, AUGguaugcc, AUGguaugcg, AUGguaugcu, AUGguaugga, AUGguauggc, AUGguaugug, AUGguauguu, AUGguauuau, AUGguauuga, AUGguauuug, AUGgucaggg, AUGgucaguc, AUGgucagug, AUGgucauuu, AUGgugaaaa, AUGgugaaac, AUGgugaaau, AUGgugaacu, AUGgugaaga, AUGgugacgu, AUGgugagaa, AUGgugagac, AUGgugagag, AUGgugagca, AUGgugagcc, AUGgugagcg, AUGgugagcu, AUGgugaggc, AUGgugaggg, AUGgugagua, AUGgugaguc, AUGgugagug, AUGgugaguu, AUGgugauuu, AUGgugcgau, AUGgugcgug, AUGgugggua, AUGgugggug, AUGguggguu, AUGgugguua, AUGguguaag, AUGgugugaa, AUGgugugua, AUGgugugug, AUGguuacuc, AUGguuagca, AUGguuaguc, AUGguuagug, AUGguuaguu, AUGguucagu, AUGguucguc, AUGguuggua, AUGguugguc, AUGguugguu, AUGguuguuu, AUGguuugca, AUGguuugua, AUUgcaagua, AUUguaaaua, AUUguaagau, AUUguaagca, AUUguaagga, AUUguaaggc, AUUguaagua, AUUguaaguc, AUUguaaguu, AUUguaauua, AUUguaauuu, AUUguacaaa, AUUguaccuc, AUUguacgug, AUUguacuug, AUUguaggua, AUUguaugag, AUUguaugua, AUUgucuguu, AUUgugagcu, AUUgugagua, AUUgugaguc, AUUgugaguu, AUUgugegug, AUUgugggug, AUUguuagug, CAAguaaaaa, CAAguaaaua, CAAguaaauc, CAAguaaaug, CAAguaaccc, CAAguaacua, CAAguaacug, CAAguaagaa, CAAguaagac, CAAguaagau, CAAguaaggu, CAAguaagua, CAAguaaguc, CAAguaagug, CAAguaaguu, CAAguaaucc, CAAguaaucu, CAAguaauua, CAAguaauuc, CAAguaauug, CAAguaauuu, CAAguacaca, CAAguacguu, CAAguacuuu, CAAguagcug, CAAguaggau, CAAguaggua, CAAguagguc, CAAguaggug, CAAguagguu, CAAguaguuu, CAAguauaac, CAAguauaug, CAAguaucuu, CAAguaugag, CAAguaugua, CAAguauguc, CAAguaugug, CAAguauguu, CAAguauuga, CAAguauuuc, CAAgucagac, CAAgucagua, CAAgucuaua, CAAgucugau, CAAgugacuu, CAAgugagaa, CAAgugagac, CAAgugagca, CAAgugaggc, CAAgugaggg, CAAgugagua, CAAgugaguc, CAAgugagug, CAAgugaucc, CAAgugaucu, CAAgugauuc, CAAgugauug, CAAgugauuu, CAAgugccuu, CAAgugggua, CAAguggguc, CAAgugggug, CAAgugugag, CAAguuaaaa, CAAguuaagu, CAAguuaauc, CAAguuagaa, CAAguuaguu, CAAguucaag, CAAguuccgu, CAAguuggua, CAAguuuagu, CAAguuucca, CAAguuuguu, CACguaagag, CACguaagca, CACguaauug, CACguaggac, CACguaucga, CACgucaguu, CACgugagcu, CACgugaguc, CACgugagug, CAGgcaagaa, CAGgcaagac, CAGgcaagag, CAGgcaagga, CAGgcaagua, CAGgcaagug, CAGgcaaguu, CAGgcacgca, CAGgcagagg, CAGgcaggug, CAGgcaucau, CAGgcaugaa, CAGgcaugag, CAGgcaugca, CAGgcaugcg, CAGgcaugug, CAGgcgagag, CAGgcgccug, CAGgcgugug, CAGguaaaaa, CAGguaaaag, CAGguaaaca, CAGguaaacc, CAGguaaaga, CAGguaaagc, CAGguaaagu, CAGguaaaua, CAGguaaauc, CAGguaaaug, CAGguaaauu, CAGguaacag, CAGguaacau, CAGguaacca, CAGguaaccg, CAGguaacgu, CAGguaacua, CAGguaacuc, CAGguaacug, CAGguaacuu, CAGguaagaa, CAGguaagac, CAGguaagag, CAGguaagau, CAGguaagcc, CAGguaagga, CAGguaaggc, CAGguaaggg, CAGguaaggu, CAGguaagua, CAGguaagug, CAGguaaguu, CAGguaauaa, CAGguaauau, CAGguaaucc, CAGguaaugc, CAGguaaugg, CAGguaaugu, CAGguaauua, CAGguaauuc, CAGguaauug, CAGguaauuu, CAGguacaaa, CAGguacaag, CAGguacaau, CAGguacaca, CAGguacacg, CAGguacaga, CAGguacagg, CAGguacagu, CAGguacaua, CAGguacaug, CAGguacauu, CAGguaccac, CAGguaccca, CAGguacccg, CAGguacccu, CAGguaccgc, CAGguaccgg, CAGguaccuc, CAGguaccug, CAGguaccuu, CAGguacgag, CAGguacgca, CAGguacgcc, CAGguacggu, CAGguacgua, CAGguacgug, CAGguacuaa, CAGguacuag, CAGguacuau, CAGguacucc, CAGguacucu, CAGguacuga, CAGguacugc, CAGguacugu, CAGguacuua, CAGguacuuu, CAGguagaaa, CAGguagaac, CAGguagaag, CAGguagaca, CAGguagacc, CAGguagaga, CAGguagauu, CAGguagcaa, CAGguagcac, CAGguagcag, CAGguagcca, CAGguagcgu, CAGguagcua, CAGguagcuc, CAGguagcug, CAGguagcuu, CAGguaggaa, CAGguaggac, CAGguaggag, CAGguaggca, CAGguaggga, CAGguagggc, CAGguagggg, CAGguagggu, CAGguaggua, CAGguagguc, CAGguaggug, CAGguagguu, CAGguaguaa, CAGguaguau, CAGguaguca, CAGguagucc, CAGguaguga, CAGguagugu, CAGguaguuc, CAGguaguug, CAGguaguuu, CAGguauaag, CAGguauaca, CAGguauaga, CAGguauauc, CAGguauaug, CAGguauauu, CAGguaucag, CAGguaucau, CAGguauccu, CAGguaucga, CAGguaucgc, CAGguaucua, CAGguaucug, CAGguaucuu, CAGguaugaa, CAGguaugac, CAGguaugag, CAGguaugau, CAGguaugca, CAGguaugcc, CAGguaugcg, CAGguaugcu, CAGguaugga, CAGguauggg, CAGguauggu, CAGguaugua, CAGguauguc, CAGguaugug, CAGguauguu, CAGguauuau, CAGguauuca, CAGguauucu, CAGguauuga, CAGguauugg, CAGguauugu, CAGguauuua, CAGguauuuc, CAGguauuug, CAGguauuuu, CAGgucaaca, CAGgucaaug, CAGgucacgu, CAGgucagaa, CAGgucagac, CAGgucagca, CAGgucagcc, CAGgucagcg, CAGgucagga, CAGgucagua, CAGgucaguc, CAGgucagug, CAGgucaguu, CAGgucaucc, CAGgucaugc, CAGgucauua, CAGgucauuu, CAGguccacc, CAGguccacu, CAGguccagu, CAGguccauc, CAGguccauu, CAGgucccag, CAGgucccug, CAGguccuga, CAGguccugc, CAGguccugg, CAGgucggcc, CAGgucggug, CAGgucguug, CAGgucucuc, CAGgucucuu, CAGgucugag, CAGgucugcc, CAGgucugcg, CAGgucugga, CAGgucuggu, CAGgucugua, CAGgucuguc, CAGgucugug, CAGgucuguu, CAGgucuucc, CAGgucuuuc, CAGgugaaag, CAGgugaaau, CAGgugaaca, CAGgugaaga, CAGgugaagg, CAGgugaaua, CAGgugaauc, CAGgugaauu, CAGgugacaa, CAGgugacau, CAGgugacca, CAGgugaccc, CAGgugaccg, CAGgugaccu, CAGgugacgg, CAGgugacua, CAGgugacuc, CAGgugacug, CAGgugagaa, CAGgugagac, CAGgugagag, CAGgugagau, CAGgugagca, CAGgugagcc, CAGgugagcg, CAGgugagcu, CAGgugagga, CAGgugaggc, CAGgugaggg, CAGgugaggu, CAGgugagua, CAGgugaguc, CAGgugagug, CAGgugaguu, CAGgugauaa, CAGgugaucc, CAGgugaucu, CAGgugaugc, CAGgugaugg, CAGgugaugu, CAGgugauua, CAGgugauuc, CAGgugauug, CAGgugauuu, CAGgugcaaa, CAGgugcaag, CAGgugcaca, CAGgugcacg, CAGgugcaga, CAGgugcagg, CAGgugcaua, CAGgugcauc, CAGgugcaug, CAGgugccaa, CAGgugccca, CAGgugcccc, CAGgugcccg, CAGgugccua, CAGgugccug, CAGgugcgaa, CAGgugcgca, CAGgugcgcc, CAGgugcgcg, CAGgugcgga, CAGgugcggu, CAGgugcgua, CAGgugcguc, CAGgugcgug, CAGgugcuag, CAGgugcuau, CAGgugcuca, CAGgugcucc, CAGgugcucg, CAGgugcugc, CAGgugcugg, CAGgugcuua, CAGgugcuuc, CAGgugcuug, CAGguggaac, CAGguggaag, CAGguggaau, CAGguggaga, CAGguggagu, CAGguggauu, CAGguggcca, CAGguggcuc, CAGguggcug, CAGgugggaa, CAGgugggac, CAGgugggag, CAGgugggau, CAGgugggca, CAGgugggcc, CAGgugggcu, CAGgugggga, CAGguggggc, CAGguggggg, CAGguggggu, CAGgugggua, CAGguggguc, CAGgugggug, CAGguggguu, CAGguggucu, CAGguggugg, CAGgugguug, CAGguguaca, CAGguguagg, CAGguguauc, CAGgugucac, CAGgugucag, CAGgugucca, CAGguguccu, CAGgugucua, CAGgugucuc, CAGgugucug, CAGgugugaa, CAGgugugac, CAGgugugag, CAGgugugau, CAGgugugca, CAGgugugcc, CAGgugugcg, CAGgugugcu, CAGgugugga, CAGguguggc, CAGgugugua, CAGguguguc, CAGgugugug, CAGguguguu, CAGguguuua, CAGguuaaaa, CAGguuaaua, CAGguuaauc, CAGguuaccu, CAGguuagaa, CAGguuagag, CAGguuagau, CAGguuagcc, CAGguuaggg, CAGguuaggu, CAGguuagua, CAGguuaguc, CAGguuagug, CAGguuaguu, CAGguuauca, CAGguuaugu, CAGguuauua, CAGguuauug, CAGguucaaa, CAGguucaac, CAGguucaag, CAGguucaca, CAGguucacg, CAGguucagg, CAGguucaug, CAGguuccag, CAGguuccca, CAGguucccg, CAGguucgaa, CAGguucgag, CAGguucuau, CAGguucugc, CAGguucuua, CAGguucuuc, CAGguucuuu, CAGguugaac, CAGguugaag, CAGguugagu, CAGguugaua, CAGguuggag, CAGguuggca, CAGguuggcc, CAGguugguc, CAGguuggug, CAGguugguu, CAGguuguaa, CAGguuguac, CAGguuguau, CAGguuguca, CAGguuguga, CAGguuguug, CAGguuuaag, CAGguuuacc, CAGguuuagc, CAGguuuagu, CAGguuucuu, CAGguuugaa, CAGguuugag, CAGguuugau, CAGguuugcc, CAGguuugcu, CAGguuuggg, CAGguuuggu, CAGguuugua, CAGguuugug, CAGguuuguu, CAGguuuucu, CAGguuuugg, CAGguuuuuc, CAGguuuuuu, CAUgcagguu, CAUguaaaac, CAUguaacua, CAUguaagaa, CAUguaagag, CAUguaagau, CAUguaagcc, CAUguaagua, CAUguaagug, CAUguaaguu, CAUguaauua, CAUguacaua, CAUguaccac, CAUguacguu, CAUguaggua, CAUguaggug, CAUguagguu, CAUguaugaa, CAUguaugua, CAUguaugug, CAUguauguu, CAUgugagaa, CAUgugagca, CAUgugagcu, CAUgugagua, CAUgugaguc, CAUgugagug, CAUgugaguu, CAUgugcgua, CAUgugggaa, CAUguggguu, CAUgugugug, CAUguguguu, CAUguuaaua, CAUguuagcc, CCAguaagau, CCAguaagca, CCAguaagcc, CCAguaagcu, CCAguaagga, CCAguaagua, CCAguaaguc, CCAguaagug, CCAguaaguu, CCAguaauug, CCAguacggg, CCAguagguc, CCAguauugu, CCAgugaggc, CCAgugagua, CCAgugagug, CCAguggguc, CCAguuaguu, CCAguugagu, CCCguaagau, CCCguauguc, CCCguauguu, CCCguccugc, CCCgugagug, CCGguaaaga, CCGguaagau, CCGguaagcc, CCGguaagga, CCGguaaggc, CCGguaaugg, CCGguacagu, CCGguacuga, CCGguauucc, CCGgucagug, CCGgugaaaa, CCGgugagaa, CCGgugaggg, CCGgugagug, CCGgugaguu, CCGgugcgcg, CCGgugggcg, CCGguugguc, CCUguaaaug, CCUguaaauu, CCUguaagaa, CCUguaagac, CCUguaagag, CCUguaagca, CCUguaagcg, CCUguaagga, CCUguaaguu, CCUguaggua, CCUguaggug, CCUguaucuu, CCUguauggu, CCUguaugug, CCUgugagaa, CCUgugagca, CCUgugaggg, CCUgugaguc, CCUgugagug, CCUgugaguu, CCUguggcuc, CCUgugggua, CCUgugugua, CCUguuagaa, CGAguaaggg, CGAguaaggu, CGAguagcug, CGAguaggug, CGAguagguu, CGAgugagca, CGCguaagag, CGGgcaggca, CGGguaagcc, CGGguaagcu, CGGguaaguu, CGGguaauuc, CGGguaauuu, CGGguacagu, CGGguacggg, CGGguaggag, CGGguaggcc, CGGguaggug, CGGguauuua, CGGgucugag, CGGgugaccg, CGGgugacuc, CGGgugagaa, CGGgugaggg, CGGgugaggu, CGGgugagua, CGGgugagug, CGGgugaguu, CGGgugauuu, CGGgugccuu, CGGgugggag, CGGgugggug, CGGguggguu, CGGguguguc, CGGgugugug, CGGguguguu, CGGguucaag, CGGguucaug, CGGguuugcu, CGUguagggu, CGUguaugca, CGUguaugua, CGUgucugua, CGUgugagug, CGUguuuucu, CUAguaaaug, CUAguaagcg, CUAguaagcu, CUAguaagua, CUAguaaguc, CUAguaagug, CUAguaaguu, CUAguaauuu, CUAguaggua, CUAguagguu, CUAguaugua, CUAguauguu, CUAgugagua, CUCguaagca, CUCguaagug, CUCguaaguu, CUCguaucug, CUCgucugug, CUCgugaaua, CUCgugagua, CUCgugauua, CUGguaaaaa, CUGguaaaau, CUGguaaacc, CUGguaaacg, CUGguaaagc, CUGguaaaua, CUGguaaauc, CUGguaaaug, CUGguaaauu, CUGguaacac, CUGguaacag, CUGguaaccc, CUGguaaccg, CUGguaacug, CUGguaacuu, CUGguaagaa, CUGguaagag, CUGguaagau, CUGguaagca, CUGguaagcc, CUGguaagcu, CUGguaagga, CUGguaaggc, CUGguaaggg, CUGguaaggu, CUGguaagua, CUGguaagug, CUGguaaguu, CUGguaauga, CUGguaaugc, CUGguaauuc, CUGguaauuu, CUGguacaac, CUGguacaau, CUGguacaga, CUGguacaua, CUGguacauu, CUGguaccau, CUGguacguu, CUGguacuaa, CUGguacuug, CUGguacuuu, CUGguagaga, CUGguagaua, CUGguagcgu, CUGguaggau, CUGguaggca, CUGguaggua, CUGguagguc, CUGguaggug, CUGguaucaa, CUGguaugau, CUGguauggc, CUGguauggu, CUGguaugua, CUGguaugug, CUGguauguu, CUGguauuga, CUGguauuuc, CUGguauuuu, CUGgucaaca, CUGgucagag, CUGgucccgc, CUGgucggua, CUGgucuggg, CUGgugaagu, CUGgugaaua, CUGgugaauu, CUGgugacua, CUGgugagaa, CUGgugagac, CUGgugagca, CUGgugagcu, CUGgugagga, CUGgugaggc, CUGgugaggg, CUGgugaggu, CUGgugagua, CUGgugaguc, CUGgugagug, CUGgugaguu, CUGgugauua, CUGgugauuu, CUGgugcaga, CUGgugcgcu, CUGgugcgug, CUGgugcuga, CUGgugggag, CUGgugggga, CUGgugggua, CUGguggguc, CUGgugggug, CUGguggguu, CUGgugugaa, CUGgugugca, CUGgugugcu, CUGguguggu, CUGgugugug, CUGguguguu, CUGguuagcu, CUGguuagug, CUGguucgug, CUGguuggcu, CUGguuguuu, CUGguuugua, CUGguuuguc, CUGguuugug, CUUguaaaug, CUUguaagcu, CUUguaagga, CUUguaaggc, CUUguaagua, CUUguaagug, CUUguaaguu, CUUguacguc, CUUguacgug, CUUguaggua, CUUguagugc, CUUguauagg, CUUgucagua, CUUgugagua, CUUgugaguc, CUUgugaguu, CUUguggguu, CUUgugugua, CUUguuagug, CUUguuugag, GAAguaaaac, GAAguaaagc, GAAguaaagu, GAAguaaaua, GAAguaaauu, GAAguaagaa, GAAguaagcc, GAAguaagcu, GAAguaagga, GAAguaagua, GAAguaagug, GAAguaaguu, GAAguaauau, GAAguaaugc, GAAguaauua, GAAguaauuu, GAAguaccau, GAAguacgua, GAAguacguc, GAAguaggca, GAAguagguc, GAAguauaaa, GAAguaugcu, GAAguaugug, GAAguauguu, GAAguauuaa, GAAgucagug, GAAgugagag, GAAgugagcg, GAAgugaggu, GAAgugaguc, GAAgugagug, GAAgugaguu, GAAgugauaa, GAAgugauuc, GAAgugcgug, GAAguguggg, GAAguguguc, GAAguuggug, GACguaaagu, GACguaagcu, GACguaagua, GACguaaugg, GACguaugcc, GACguauguu, GACgugagcc, GACgugagug, GAGgcaaaug, GAGgcaagag, GAGgcaagua, GAGgcaagug, GAGgcaaguu, GAGgcacgag, GAGgcaggga, GAGgcaugug, GAGgcgaagg, GAGguaaaaa, GAGguaaaac, GAGguaaaag, GAGguaaaau, GAGguaaacc, GAGguaaaga, GAGguaaagc, GAGguaaagu, GAGguaaaua, GAGguaaauc, GAGguaaaug, GAGguaaauu, GAGguaacaa, GAGguaacag, GAGguaacca, GAGguaaccu, GAGguaacuu, GAGguaagaa, GAGguaagag, GAGguaagau, GAGguaagca, GAGguaagcc, GAGguaagcg, GAGguaagcu, GAGguaagga, GAGguaaggc, GAGguaaggg, GAGguaaggu, GAGguaagua, GAGguaaguc, GAGguaauaa, GAGguaauac, GAGguaauau, GAGguaauca, GAGguaaucu, GAGguaaugg, GAGguaaugu, GAGguaauug, GAGguaauuu, GAGguacaaa, GAGguacaac, GAGguacaga, GAGguacagc, GAGguacagu, GAGguacaua, GAGguacauu, GAGguaccag, GAGguaccga, GAGguaccug, GAGguaccuu, GAGguacuag, GAGguacuau, GAGguacucc, GAGguacugc, GAGguacugg, GAGguacugu, GAGguacuug, GAGguacuuu, GAGguagaag, GAGguagaga, GAGguagagg, GAGguagagu, GAGguagauc, GAGguagcua, GAGguagcug, GAGguaggaa, GAGguaggag, GAGguaggca, GAGguaggcu, GAGguaggga, GAGguagggc, GAGguagggg, GAGguaggua, GAGguaggug, GAGguagguu, GAGguaguaa, GAGguaguag, GAGguaguau, GAGguagucu, GAGguagugc, GAGguagugg, GAGguaguua, GAGguaguug, GAGguauaag, GAGguauacu, GAGguauagc, GAGguauaug, GAGguauauu, GAGguaucau, GAGguaucug, GAGguaucuu, GAGguaugaa, GAGguaugac, GAGguaugag, GAGguaugcc, GAGguaugcg, GAGguaugcu, GAGguaugga, GAGguauggg, GAGguauggu, GAGguaugua, GAGguauguc, GAGguaugug, GAGguauguu, GAGguauucc, GAGguauuga, GAGguauugu, GAGguauuua, GAGguauuuc, GAGguauuug, GAGguauuuu, GAGgucaaca, GAGgucaagg, GAGgucaaug, GAGgucacug, GAGgucagaa, GAGgucagag, GAGgucagcu, GAGgucagga, GAGgucaggc, GAGgucaggg, GAGgucaggu, GAGgucagua, GAGgucauau, GAGgucaugu, GAGgucauuu, GAGguccaua, GAGguccauc, GAGguccggg, GAGguccggu, GAGguccuug, GAGgucgggg, GAGgucucgu, GAGgucugag, GAGgucuggu, GAGgucuguc, GAGgucuguu, GAGgucuuuu, GAGgugaaaa, GAGgugaaau, GAGgugaaca, GAGgugaagg, GAGgugaaua, GAGgugaauu, GAGgugacau, GAGgugacca, GAGgugaccu, GAGgugacua, GAGgugacuu, GAGgugagaa, GAGgugagac, GAGgugagag, GAGgugagau, GAGgugagca, GAGgugagcc, GAGgugagcg, GAGgugagcu, GAGgugagga, GAGgugaggc, GAGgugaggg, GAGgugagua, GAGgugagug, GAGgugaguu, GAGgugauau, GAGgugaucc, GAGgugaucu, GAGgugauga, GAGgugaugg, GAGgugaugu, GAGgugauuc, GAGgugcaca, GAGgugcaga, GAGgugcagc, GAGgugcagg, GAGgugccag, GAGgugccca, GAGgugccuu, GAGgugcggg, GAGgugcgug, GAGgugcucc, GAGgugcugg, GAGgugcuua, GAGgugcuug, GAGguggaaa, GAGguggaau, GAGguggacc, GAGguggacg, GAGguggagg, GAGguggcug, GAGgugggaa, GAGgugggag, GAGgugggau, GAGgugggca, GAGgugggcg, GAGgugggcu, GAGgugggga, GAGguggggc, GAGguggggg, GAGgugggua, GAGguggguc, GAGgugggug, GAGguggguu, GAGgugguau, GAGgugguuc, GAGgugucau, GAGgugugag, GAGgugugau, GAGgugugca, GAGgugugcu, GAGgugugga, GAGguguggg, GAGguguggu, GAGgugugua, GAGgugugug, GAGguuaaau, GAGguuaaga, GAGguuaaua, GAGguuaccg, GAGguuagaa, GAGguuagac, GAGguuagag, GAGguuaggu, GAGguuagua, GAGguuaguc, GAGguuagug, GAGguuaguu, GAGguuaugu, GAGguuauuc, GAGguucaaa, GAGguucaua, GAGguucuga, GAGguugaag, GAGguugcag, GAGguugcug, GAGguuggaa, GAGguuggag, GAGguuggau, GAGguuggua, GAGguugguc, GAGguugguu, GAGguuguag, GAGguuucug, GAGguuugag, GAGguuugga, GAGguuuggg, GAGguuugua, GAGguuuguu, GAGguuuuca, GAGguuuuga, GAGguuuugg, GAGguuuuua, GAGguuuuuc, GAUguaaaau, GAUguaagca, GAUguaagcc, GAUguaaggu, GAUguaagua, GAUguaagug, GAUguaaguu, GAUguacauc, GAUguaggua, GAUguauggc, GAUguaugua, GAUguauguu, GAUgucagug, GAUgugagag, GAUgugagcc, GAUgugagcu, GAUgugagga, GAUgugaguc, GAUgugagug, GAUgugaguu, GAUgugggua, GAUgugggug, GAUguguguu, GAUguuagcu, GAUguucagu, GAUguucgug, GAUguuuguu, GCAguaaagg, GCAguaagaa, GCAguaagga, GCAguaagua, GCAguaaguc, GCAguaaguu, GCAguagaug, GCAguaggua, GCAguaugug, GCAguauguu, GCAgucagua, GCAgucagug, GCAguccggu, GCAgugacuu, GCAgugagcc, GCAgugagcg, GCAgugagcu, GCAgugagua, GCAgugagug, GCAgugaguu, GCAgugggua, GCAguuaagu, GCAguugagu, GCCguaaguc, GCCgugagua, GCGguaaagc, GCGguaaaua, GCGguaagcu, GCGguaaggg, GCGguaagug, GCGguaauca, GCGguacgua, GCGguacuug, GCGguagggu, GCGguagugu, GCGgugagca, GCGgugagcu, GCGgugaguu, GCGguggcuc, GCGgugugca, GCGguguguu, GCGguuaagu, GCGguuugca, GCUgcuguaa, GCUguaaaua, GCUguaagac, GCUguaagag, GCUguaagca, GCUguaagga, GCUguaagua, GCUguaaguc, GCUguaagug, GCUguaaguu, GCUguaggug, GCUguauggu, GCUgucagug, GCUguccuug, GCUgugagaa, GCUgugagcc, GCUgugagga, GCUgugagua, GCUgugaguc, GCUgugagug, GCUgugaguu, GCUguggguu, GGAguaagag, GGAguaagca, GGAguaagcc, GGAguaagcu, GGAguaagga, GGAguaagug, GGAguaaguu, GGAguaauuu, GGAguacugu, GGAguaggaa, GGAguaggua, GGAguagguu, GGAguaguau, GGAguaugac, GGAguauggu, GGAgucaagu, GGAgugaggg, GGAgugagua, GGAgugaguc, GGAgugagug, GGAgugaguu, GGAgugcuuu, GGAgugggca, GGAgugggug, GGAguuaagg, GGAguugaga, GGCguaagcc, GGCguaggua, GGCguaggug, GGCgugagcc, GGCgugaguc, GGGguaaaca, GGGguaaacc, GGGguaaacu, GGGguaagaa, GGGguaagag, GGGguaagau, GGGguaagca, GGGguaagcc, GGGguaagcu, GGGguaagga, GGGguaaggg, GGGguaagua, GGGguaagug, GGGguaaguu, GGGguagaca, GGGguaggag, GGGguaggcc, GGGguaggga, GGGguaggua, GGGguaggug, GGGguagguu, GGGguagugc, GGGguaucug, GGGguaugac, GGGguaugga, GGGguaugua, GGGguauguc, GGGguaugug, GGGguauguu, GGGgucagua, GGGguccgug, GGGgucggag, GGGgucugug, GGGgugaaca, GGGgugaaga, GGGgugagaa, GGGgugagau, GGGgugagcc, GGGgugagcg, GGGgugagcu, GGGgugagga, GGGgugaggc, GGGgugaggg, GGGgugaguc, GGGgugagug, GGGgugaguu, GGGgugcgua, GGGguggggu, GGGgugggua, GGGgugggug, GGGguggguu, GGGgugugcg, GGGgugugua, GGGguguguc, GGGgugugug, GGGguuacag, GGGguuggac, GGGguuggga, GGGguuugcc, GGGguuugua, GGUguaagaa, GGUguaagau, GGUguaagca, GGUguaagcc, GGUguaagcg, GGUguaaguc, GGUguaagug, GGUguagguc, GGUguaggug, GGUguagguu, GGUguccgua, GGUgugagag, GGUgugagcc, GGUgugagcu, GGUgugagua, GGUgugaguc, GGUgugcuuc, GGUguggcug, GGUgugguga, GGUgugucug, GGUguugaaa, GGUguugcug, GUAguaagau, GUAguaagua, GUAguaagug, GUAguagcuu, GUAguaggua, GUAgucagua, GUAgugagua, GUAguggugg, GUAguuaagu, GUAguuucug, GUCguaagug, GUCgugagug, GUCgugaguu, GUGgcaagua, GUGgcuugua, GUGguaaaau, GUGguaaaga, GUGguaaauu, GUGguaacau, GUGguaacua, GUGguaagaa, GUGguaagac, GUGguaagag, GUGguaagau, GUGguaagca, GUGguaagcg, GUGguaagcu, GUGguaagga, GUGguaaggc, GUGguaagua, GUGguaaguc, GUGguaagug, GUGguaaguu, GUGguaauga, GUGguaauuc, GUGguaauuu, GUGguacaug, GUGguacgau, GUGguacuau, GUGguacuug, GUGguagaua, GUGguagege, GUGguaggga, GUGguagguc, GUGguaggug, GUGguagguu, GUGguauaaa, GUGguaucuc, GUGguaugaa, GUGguaugau, GUGguaugca, GUGguaugua, GUGguauguu, GUGguccgug, GUGgucuggc, GUGgugaaac, GUGgugagaa, GUGgugagau, GUGgugagca, GUGgugagcu, GUGgugagga, GUGgugaggc, GUGgugagug, GUGgugaguu, GUGgugauua, GUGgugauuc, GUGgugcgau, GUGgugcuua, GUGgugggaa, GUGgugggua, GUGguggguc, GUGguguccg, GUGguuagca, GUGguuaggu, GUGguuagug, GUGguuugca, GUGguuugua, GUUguaaggu, GUUguaagua, GUUguaaguc, GUUguaaguu, GUUguaccac, GUUguagcgu, GUUguaugug, GUUguauguu, GUUgucugug, GUUgugagcu, GUUgugagug, GUUgugaguu, GUUgugggua, GUUguggguu, UAAguaaaug, UAAguaacua, UAAguaagaa, UAAguaagag, UAAguaagau, UAAguaagca, UAAguaagcu, UAAguaagga, UAAguaaggu, UAAguaagua, UAAguaaguc, UAAguaagug, UAAguaaguu, UAAguaauaa, UAAguacuag, UAAguaguuu, UAAguauaaa, UAAguauaca, UAAguaugua, UAAguauuau, UAAguauuuu, UAAgucuuuu, UAAgugagac, UAAgugagga, UAAgugaggg, UAAgugagua, UAAgugaguc, UAAgugagug, UAAgugaguu, UAAgugaucc, UAAgugauuc, UAAgugcgug, UAAguuaagu, UAAguuccag, UAAguucuuu, UAAguuguaa, UAAguuguau, UAAguuuguu, UACguaacug, UACguaagaa, UACguaagau, UACguaagua, UACguaagug, UACguauccu, UACgucuggc, UACgugacca, UAGgcaagac, UAGgcaaguc, UAGgcagguc, UAGgcgugug, UAGguaaaaa, UAGguaaaac, UAGguaaaag, UAGguaaaau, UAGguaaaca, UAGguaaaga, UAGguaaaua, UAGguaaauc, UAGguaaaug, UAGguaaauu, UAGguaacac, UAGguaacag, UAGguaacau, UAGguaacca, UAGguaacgg, UAGguaacua, UAGguaacuc, UAGguaacug, UAGguaacuu, UAGguaagac, UAGguaagag, UAGguaagau, UAGguaagca, UAGguaagcc, UAGguaagcu, UAGguaagga, UAGguaaggc, UAGguaaggg, UAGguaagua, UAGguaaguc, UAGguaagug, UAGguaaguu, UAGguaauag, UAGguaauau, UAGguaaucu, UAGguaauga, UAGguaaugg, UAGguaaugu, UAGguaauua, UAGguaauuc, UAGguaauuu, UAGguacagc, UAGguacagu, UAGguacauu, UAGguaccag, UAGguaccua, UAGguaccuu, UAGguacgag, UAGguacgua, UAGguacguu, UAGguacuau, UAGguacuga, UAGguacugg, UAGguacuuc, UAGguacuuu, UAGguagcgg, UAGguaggaa, UAGguaggac, UAGguaggau, UAGguaggga, UAGguagggg, UAGguaggua, UAGguagguc, UAGguaggug, UAGguagguu, UAGguaguaa, UAGguagucu, UAGguagugg, UAGguagugu, UAGguaguuu, UAGguauaaa, UAGguauaac, UAGguauaag, UAGguauaau, UAGguauaca, UAGguauacu, UAGguauaua, UAGguauauc, UAGguauauu, UAGguaucag, UAGguaucua, UAGguaucuc, UAGguaugaa, UAGguaugag, UAGguaugca, UAGguaugga, UAGguauggc, UAGguauggu, UAGguaugua, UAGguauguc, UAGguaugug, UAGguauguu, UAGguauuaa, UAGguauuac, UAGguauuau, UAGguauuca, UAGguauucc, UAGguauucu, UAGguauuga, UAGguauuua, UAGguauuuc, UAGguauuuu, UAGgucacuc, UAGgucagcu, UAGgucaggu, UAGgucagua, UAGgucagug, UAGgucaguu, UAGgucaucu, UAGgucauug, UAGguccaau, UAGguccugu, UAGgucucaa, UAGgucucgc, UAGgucuggc, UAGgucuguc, UAGgucugug, UAGgugaagu, UAGgugaaua, UAGgugaaug, UAGgugaauu, UAGgugacau, UAGgugacca, UAGgugacua, UAGgugagaa, UAGgugagac, UAGgugagag, UAGgugagau, UAGgugagcc, UAGgugagcu, UAGgugagga, UAGgugaggc, UAGgugaggu, UAGgugagua, UAGgugaguc, UAGgugagug, UAGgugauca, UAGgugauuc, UAGgugauuu, UAGgugcaua, UAGgugcauc, UAGgugccgu, UAGgugccug, UAGgugcgca, UAGgugcgua, UAGgugcgug, UAGgugcuga, UAGguggaua, UAGgugggaa, UAGgugggac, UAGgugggag, UAGgugggau, UAGgugggcc, UAGgugggcu, UAGguggguu, UAGguggugu, UAGguguaaa, UAGgugugaa, UAGgugugag, UAGgugugca, UAGgugugcc, UAGgugugcg, UAGguguggu, UAGgugugua, UAGgugugug, UAGguguugg, UAGguuaagc, UAGguuagac, UAGguuagcc, UAGguuaggc, UAGguuagua, UAGguuaguc, UAGguuagug, UAGguucccc, UAGguucuac, UAGguuggua, UAGguugguu, UAGguugucc, UAGguuuauu, UAGguuugcc, UAGguuugua, UAGguuuguc, UAGguuugug, UAGguuuguu, UAGguuuuuc, UAGguuuuug, UAUguaagaa, UAUguaagau, UAUguaagca, UAUguaagcc, UAUguaagua, UAUguaaguc, UAUguaagug, UAUguaaguu, UAUguacgug, UAUguacguu, UAUguagguc, UAUguagguu, UAUguauccu, UAUguaucuc, UAUguaugua, UAUguauguc, UAUguaugug, UAUguauuau, UAUgucagaa, UAUgucugua, UAUgugaaua, UAUgugacag, UAUgugagua, UAUgugagug, UAUgugaguu, UAUgugggca, UAUgugugua, UAUguguuua, UAUguuuugu, UCAgcgacau, UCAguaaaau, UCAguaaaua, UCAguaacug, UCAguaagaa, UCAguaagag, UCAguaagau, UCAguaagca, UCAguaagcc, UCAguaagcu, UCAguaaggg, UCAguaagua, UCAguaaguc, UCAguaagug, UCAguaaguu, UCAguaucuu, UCAguaugga, UCAguauggu, UCAgucccca, UCAgugagca, UCAgugagcu, UCAgugagua, UCAgugagug, UCAgugaguu, UCAgugauug, UCAgugggug, UCAguugagc, UCAguugauu, UCAguuuagu, UCCguaagca, UCCguaagcu, UCCguaaguc, UCCguaagug, UCCguaauag, UCCguacuua, UCCguaugua, UCCguauguu, UCCgugagau, UCCgugaguc, UCGguaaauu, UCGguaagag, UCGguaagcu, UCGguacauc, UCGguacucc, UCGguagacc, UCGguagguu, UCGguaguaa, UCGguaugug, UCGguauguu, UCGguauuga, UCGgucagua, UCGgucuuag, UCGgugaagu, UCGgugagaa, UCGgugagca, UCGgugaggc, UCGgugagua, UCGgugcgcu, UCGgugcuuu, UCGgugguuu, UCGguuagcu, UCUguaaaag, UCUguaagaa, UCUguaagau, UCUguaagca, UCUguaagcu, UCUguaagua, UCUguaaguc, UCUguaagug, UCUguaaguu, UCUguaauaa, UCUguaauga, UCUguaaugu, UCUguaggua, UCUguagguu, UCUguauaua, UCUguaugac, UCUguaugua, UCUguccueg, UCUgugagag, UCUgugagcu, UCUgugagga, UCUgugagua, UCUgugaguc, UCUgugagug, UCUgugaguu, UCUgugcgua, UCUgugugag, UGAguaacuu, UGAguaagau, UGAguaagca, UGAguaagcu, UGAguaaggc, UGAguaaggu, UGAguaagua, UGAguaaguc, UGAguaagug, UGAguaaguu, UGAguaaucc, UGAguaauua, UGAguacagu, UGAguacgua, UGAguacguu, UGAguacugu, UGAguagcug, UGAguaggua, UGAguauaaa, UGAguaugcu, UGAguaugga, UGAguaugua, UGAguauguc, UGAguauguu, UGAgucagag, UGAgucuacg, UGAgugaaua, UGAgugaauu, UGAgugagaa, UGAgugagau, UGAgugagca, UGAgugagcc, UGAgugagga, UGAgugagua, UGAgugagug, UGAgugaguu, UGAgugggaa, UGAguuaaga, UGAguuaaug, UGAguuacgg, UGAguuaggu, UGAguucuau, UGAguugguu, UGAguuguag, UGAguuuauc, UGCguaaguc, UGCguaagug, UGCguacggc, UGCguacggg, UGCguaugua, UGGgcaaguc, UGGgcaagug, UGGgcacauc, UGGgccacgu, UGGgccccgg, UGGguaaaau, UGGguaaagc, UGGguaaagg, UGGguaaagu, UGGguaaaua, UGGguaaaug, UGGguaaauu, UGGguaacag, UGGguaacau, UGGguaacua, UGGguaacuu, UGGguaagaa, UGGguaagac, UGGguaagag, UGGguaagau, UGGguaagca, UGGguaagcc, UGGguaagcu, UGGguaaggg, UGGguaaggu, UGGguaagua, UGGguaaguc, UGGguaagug, UGGguaaguu, UGGguaaugu, UGGguaauua, UGGguaauuu, UGGguacaaa, UGGguacagu, UGGguacuac, UGGguaggga, UGGguagguc, UGGguaggug, UGGguagguu, UGGguaguua, UGGguauagu, UGGguaugaa, UGGguaugac, UGGguaugag, UGGguaugua, UGGguauguc, UGGguaugug, UGGguauguu, UGGguauuug, UGGgucuuug, UGGgugaccu, UGGgugacua, UGGgugagac, UGGgugagag, UGGgugagca, UGGgugagcc, UGGgugagga, UGGgugaggc, UGGgugaggg, UGGgugagua, UGGgugaguc, UGGgugagug, UGGgugaguu, UGGgugcgug, UGGguggagg, UGGguggcuu, UGGguggggg, UGGgugggua, UGGguggguc, UGGgugggug, UGGguggguu, UGGgugugga, UGGguguguc, UGGgugugug, UGGguguguu, UGGguguuua, UGGguuaaug, UGGguuaguc, UGGguuagug, UGGguuaguu, UGGguucaag, UGGguucgua, UGGguuggug, UGGguuuaag, UGGguuugua, UGUgcaagua, UGUguaaaua, UGUguaagaa, UGUguaagac, UGUguaagag, UGUguaaggu, UGUguaagua, UGUguaaguc, UGUguaaguu, UGUguacuuc, UGUguaggcg, UGUguaggua, UGUguaguua, UGUguaugug, UGUgucagua, UGUgucugua, UGUgucuguc, UGUgugaccc, UGUgugagau, UGUgugagca, UGUgugagcc, UGUgugagua, UGUgugaguc, UGUgugagug, UGUgugcgug, UGUgugggug, UGUguggguu, UGUgugugag, UGUguguucu, UGUguuuaga, UUAguaaaua, UUAguaagaa, UUAguaagua, UUAguaagug, UUAguaaguu, UUAguaggug, UUAgugagca, UUAgugaguu, UUAguuaagu, UUCguaaguc, UUCguaaguu, UUCguaauua, UUCgugagua, UUCgugaguu, UUGgcaagug, UUGgccgagu, UUGguaaaaa, UUGguaaaau, UUGguaaaga, UUGguaaagg, UUGguaaagu, UUGguaaauc, UUGguaaaug, UUGguaaauu, UUGguaacug, UUGguaacuu, UUGguaagaa, UUGguaagag, UUGguaagcu, UUGguaagga, UUGguaaggg, UUGguaagua, UUGguaagug, UUGguaaguu, UUGguaauac, UUGguaauca, UUGguaaugc, UUGguaaugu, UUGguaauug, UUGguaauuu, UUGguacaua, UUGguacgug, UUGguagagg, UUGguaggac, UUGguaggcg, UUGguaggcu, UUGguaggga, UUGguaggua, UUGguagguc, UUGguaggug, UUGguauaaa, UUGguauaca, UUGguauauu, UUGguaucua, UUGguaucuc, UUGguaugca, UUGguaugua, UUGguaugug, UUGguauguu, UUGguauugu, UUGguauuua, UUGguauuuu, UUGgucagaa, UUGgucagua, UUGgucucug, UUGgucugca, UUGgugaaaa, UUGgugacug, UUGgugagac, UUGgugagau, UUGgugagca, UUGgugagga, UUGgugaggg, UUGgugagua, UUGgugaguc, UUGgugagug, UUGgugaguu, UUGgugaugg, UUGgugauua, UUGgugauug, UUGgugcaca, UUGgugggaa, UUGguggggc, UUGgugggua, UUGguggguc, UUGgugggug, UUGguggguu, UUGguguggu, UUGguguguc, UUGgugugug, UUGguguguu, UUGguuaagu, UUGguuagca, UUGguuagug, UUGguuaguu, UUGguuggga, UUGguugguu, UUGguuugua, UUGguuuguc, UUUgcaagug, UUUguaaaua, UUUguaaaug, UUUguaagaa, UUUguaagac, UUUguaagag, UUUguaagca, UUUguaaggu, UUUguaagua, UUUguaaguc, UUUguaagug, UUUguaaguu, UUUguaauuu, UUUguacagg, UUUguacgug, UUUguacuag, UUUguacugu, UUUguagguu, UUUguauccu, UUUguauguu, UUUgugagca, UUUgugagug, UUUgugcguc, UUUguguguc, and uGGguaccug.

Additional exemplary gene sequences and splice site sequences (e.g., 5′ splice site sequences) include AAGgcaagau, AUGguaugug, GGGgugaggc, CAGguaggug, AAGgucagua, AAGguuagag, AUGgcacuua, UAAguaaguc, UGGgugagcu, CGAgcugggc, AAAgcacccc, UAGguggggg, AGAguaacgu, UCGgugaugu, AAUgucaguu, AGGgucugag, GAGgugacug, AUGguagguu, GAGgucuguc, CAGguaugug, CAAguacugc, CACgugcgua, CCGgugagcu, CAGguacuuc, CAGgcgagag, GAAgcaagua, AGGgugagca, CAGgcaaguc, AAGgugaggc, CAGguaagua, CCAguugggu, AAGguguggg, CAGguuggag, CCGguaugaa, UGGguaaugu, CAGgugaggu, AGAguaauag, CAGguaugag, AUGguaaguu, UUGguggguc, UUUguaagca, CUCguaugcc, UAGguaagag, UAGgcaaguu, GGAguuaagu, GAGguaugcc, AAGguguggu, CAGgugggug, UUAguaagua, AAGguuggcu, UGAguaugug, CCAgccuucc, CCUguacgug, CCUguaggua, CAGguacgcu, GAGguucuuc, AAGguugccu, CGUguucacu, CGGgugggga, UAGgugggau, CGGguaagga, AAGguacuau, GGGguaagcu, ACGguagagc, CAGgugaaga, GCGguaagag, CAGguguugu, GAAguuugug, AUGgugagca, CGGguucgug, AUUguccggc, GAUgugugug, AUGgucuguu, AAGguaggau, CCGguaagau, AAGguaaaga, GGGgugaguu, AGGguuggug, GGAgugagug, AGUguaagga, UAGguaacug, AAGgugaaga, UGGguaagug, CAGguaagag, UAGgugagcg, GAGguaaaaa, GCCguaaguu, AAGguuuugu, CAGgugagga, ACAgcccaug, GCGgugagcc, CAGguaugca, AUGguaccua, CAAguaugua, AUGgugguge, UAAguggcag, UAGguauagu, CUGguauuua, AGGguaaacg, AUAguaagug, UUGguacuga, GGUguaagcc, GAGguggaua, GAUguaagaa, ACGgucaguu, UAAguaaaca, AAGguaucug, AGGguauuug, AAGgugaaug, CUGgugaauu, CAGguuuuuu, CAUguaugug, UUGguagagg, AAGguaugcc, CAGgugccac, UCGguauuga, AAGguuugug, AAUguacagg, CAUguggguu, CAUgugaguu, UUGguaaugu, AGUguaggug, GAGguaacuc, GAGguggcgc, CUGguaauug, GAGguuugcu, UGUguacgug, UAGguaaaga, CUAguaggca, UCUgugaguc, UCUguaaggc, CAGguuugug, GAGguagggc, AAGguaacca, ACUgugaguu, UAGguaauag, AAAguaagcu, AUGgugagug, UAGguuugug, AACguaggac, GUAgcaggua, GAGgucagac, AGGguaugaa, GAGguuagug, CAGgcacgug, GGGgcaagac, CAGguguguc, CAGguauuga, CAGguauguc, AAGgcaaggu, UUGgugagaa, AAGguaaaau, GGGguaagua, AAGguaucuu, GACgugaguc, UAUguaugcu, AAGguacugu, CAGgugaacu, CACguaaaug, AAGgugugau, GAAguauuug, AAGgucugug, AAGguggagg, AAGguauaug, CAGguucuua, AGGguaacca, CAGgugucac, AAAguucugu, UUGgugaguu, CAAgugaguc, UAGguagguc, GCGgugagcu, AUUgugagga, CAGgugcaca, CAGguuggaa, CUGgucacuu, GGAguaagug, GAGgugggcu, AAGguacuug, AGGguaggau, AAUguguguu, ACAguuaagu, GAGgugugug, AAGgcgggcu, AUAgcaagua, AAGguuguua, CAAgcaaggc, GUGguaauua, UCUguucagu, AGGguaggcc, AAGguaucau, UAGguaccuu, AAGguaugac, GGAguaggua, UAAguuggca, AGUgugaggc, GAGguuugug, UGGgucugcu, CAGgugaucc, CAGgucagug, AAGguaaggg, CAGgugcagu, GAGguggguc, GCUgugagug, AAGguggagu, GGGgucaguu, AGCguaagug, AGAguaugaa, GGGguagggu, AAGgccagca, CGAguaugcc, GUGgugagcg, AAUguaaauu, CAGgugcgca, GGUguaugaa, CUUgugaguu, AAGguaucuc, AGAguaagga, UAGguaagac, GAGgugagug, CAGguguguu, UUGgugagua, AGGgcgaguu, CAGguuuugc, UUUgugaguu, AGGguaagca, GAGguccucu, CCAgcaggua, GAGguucgcg, CAGgugaucu, ACUguaagua, AAGguaaauc, CAGgcaaaua, GUGguaagca, CAGguuaaau, UUGguaauaa, UAUguaggua, CAGguaguau, AAGgugugcc, UGGguaagag, CAGgcaagca, UUGguaaggg, AAGgcaggug, ACGguaaaug, GCUgugagca, AUGguacaca, GUAguguguu, ACUguaagag, CCCgcagguc, GAGgugagcc, GAGgugcugu, UAAguaugcu, GAGgccaucu, UCAgugagug, CAGgugcuac, AAUgugggug, GAGgugugaa, CUGguagguc, GUGgcgcgcg, CAGgugcaaa, UAAguggagg, CAUgugggua, GAGguagggu, AAAgugaguu, AGGguucuag, UGUgugagcu, AGGgugaauc, CAGgucaggg, AAGgucccug, CUGguagagu, UAGgucaguu, AAAguaaggg, CAAguaugug, CAGgugcuuu, AAGguaauuc, GGGgugcacg, ACUgugcuac, CAGguaccua, CAGguagcuu, UGGgugaggc, CUGguacauu, AGGguaaucu, CAGguacaag, CAGguaauuc, AGGgcacuug, UAGgugagaa, GAGguaaugc, CCAgugaguu, AAAguaugug, CUGgugaauc, UAUguaugua, CCUgcaggug, CAGguaucug, GAGgugaggu, CUGguaaaac, UGUgugugcu, CAGguuaagu, CAGguaaucc, UAGguauuug, UGGguagguc, CAGguaacag, AGCgugcgug, AAGgucagga, GGUgugagcc, CUGguaagua, GGGgugggca, AAGgugggaa, CAGgugagug, CUGguuguua, CAGguaauag, UAGgugaguu, AGAguaaguu, UAGguaaucc, CCGgugacug, GUCgugauua, CUUguaagug, UAGguaguca, CUGguaaguc, AGGgugagcg, CAGguaugga, AUUgugacca, GUUgugggua, AAGguacaag, CUAgcaagug, CUGgugagau, CAGgugggca, AUGgcucgag, CUGguacguu, UUGgugugua, GAGgugucug, GAGgugggac, GGGgugggag, GCAgcgugag, GAGguaaaga, GAGguaugua, AAGgugagac, AAGguacaau, CUGguaugag, AACguaaaau, GUGguaggga, CUGguaugug, CUUguaagca, AAGguaggga, AUUguaagcc, AUGguaagcu, CAGgugaauu, UAGgugaaua, CAAguaugga, AUGguauggc, GAGgucaugc, CAGguacccu, ACAgugagac, CAGgucugau, GAAguugggu, CUGgugcgug, CAGguacgag, ACAgugagcc, AAGguaagua, GGAguaaggc, GAGgugugua, AAGgucauuu, CAGguagucu, AUGguaucug, AAGguaaacu, GAGguaggug, CUGguaagca, AGGguaagag, AAAguaaagc, CAGguuugag, GAGgcgggua, CGAguacgau, CAGguuguug, AAAguauggg, UAGgcugguc, AAGguaagga, AAGguuuccu, UUGguaaaac, GAGguaagua, CAGguucaag, UGGguuaugu, GAGgugaguu, ACGgugaaac, GAUguaacca, AAGgugcggg, CCGguacgug, GAUgugagaa, GUGgcgguga, CAGguauuag, GAGguuggga, AAGgcuagua, AAGgugggcg, CAGgcaggga, AAUguuaguu, GAGguaaagg, CAGgugugcu, CUGguaugau, AUGguuaguc, CUGgugagaa, CAGgccggcg, CAGgugacug, AAAguaaggu, UAAguacuug, AAGguaaagc, UCGguagggg, CAGguaggaa, AGUguaagca, CCCgugagau, GUGguuguuu, CAGguuugcc, AGGguauggg, UAAguaagug, GAGguaagac, GAUguagguc, CAAguaggug, AUAguaaaua, GAGguugggg, GAGgcgagua, CAGguagugu, GUGguaggug, CAAgugagug, AAGgugacaa, CCAgcguaau, ACGgugaggu, GGGguauauu, CAGgugagua, AAGgugcgug, UAUguaaauu, CAGgucagua, ACGguacuua, GAGgucagca, UAAguaugua, GGGgucagac, AAUgugugag, UCCgucagua, CAGgugcuuc, CCAguuagug, CCGgugggcg, AGGgugcaug, GGGguaggau, UAGgugggcc, GAGguguucg, UUGgcaagaa, UCCguaagua, CAGguguaag, CUCgugagua, GAGguguuuu, GAGgugagca, GAGguaaagu, AAGguacguu, CAGguccagu, AUGgugaaac, GUAgugagcu, CAGgugaaaa, AGGguacagg, AAGguaacgc, AAGguauacc, CCUgugagau, GGGguacgug, GAGguauggu, UAGguauuau, GAAguaggag, UCGguaaggg, CCGguaagcg, GAAguaauua, CAGgugaguc, AAGgucaaga, AUGguaaguc, CAGgugagcu, CCAguuuuug, CAGgugggag, AAGguauuau, AAGguaaaua, AAGgugcugu, AAAguacacc, CUGguucgug, UCAguaaguc, GAAguacgug, CAGgugacaa, UGGguaagaa, UGUguagggg, GAGguaggca, UUGgugaggc, AUGgugugua, CAGguccucc, UUGguaaaug, GCUgugaguu, AUGgucugua, CAUgcaggug, CUGguacacc, CAGguccuua, CAAguaaucu, AUGgcagccu, AAGgucagaa, AACgugaggc, CAGgcacgca, ACGguccagg, UCUguacaua, GAGgugauua, ACGguaaaua, AUGguaacug, CAGgcgcguu, CAGguauaga, AAGguuuguu, CAGguaugaa, UAGguuggua, CUGgugagac, CAGguuagga, AUGgugacug, UUGguauccc, CUUguaggac, AAAguguguu, CAGguuucuu, GGGguauggc, GGGguaggac, ACUguaaguc, AUCguaagcu, UAGguucccc, GGUgugagca, CUGguuggua, GGGguuaggg, UGAguaagaa, GAGguauucc, UGGguuaguc, CAGgcucgug, UAGguagagu, UAGgugcccu, AAAgugagua, GAGguucaua, UUGguaagag, ACCgugugua, UAUguaguau, UGGguaauag, CAGgucugaa, AAAguauaaa, GUGgugaguc, AGUgugauua, UUGgugugug, CAGgugaugg, GCUgugagua, CAGguacaug, AAGguacagu, GAAguuguag, CAGgugauua, UAGgugaauu, GGUguuaaua, CAGguauuua, CAAguacucg, CAAguaagaa, AAGguaccuu, ACGgugaggg, UGAgcaggca, GGGgugaccg, GAGguaaaug, CGGguuugug, AAGgugagcg, GUGguaugga, CUGguaagga, GAGguaccag, CCGgugagug, AAGguuagaa, GAGguacuug, AGAguaaaac, UCUgugagua, AAGgcgggaa, CAGguaugcg, AGGguaaaac, AAGgugacug, AGGguauguu, AAGguaugua, CAGgucucuc, CAGgcaugua, CUGguaggua, AAGgucaugc, CAGguacaca, GAUguacguu, ACAguacgug, ACGguaccca, CAGguagugc, ACAguaagag, GGUgcacacc, GAGguguaac, AAGgugugua, UAGguacuua, GCGguacugc, UGGguaaguc, CAUguaggua, CAGguaggau, CAGgucuggc, GUGguuuuaa, CAGgugggaa, UGGgugagua, CGAgugagcc, AAGguauggc, AGUguuguca, CAGgugauuu, UAGguaucuc, UAAguauguu, AAGguugagc, AGAguaaaga, GGUguaagua, GGGgugagcu, CAGguauaau, GAGguacaaa, AUGguaccaa, UAGguagggg, UGAgucagaa, AAGgcaauua, UUGguaagau, CAGguacaga, AGAguuagag, CAGgugcguc, GAGguauuac, ACGguacaga, CAGgucuucc, AAGguaaggu, GAGguaauuu, AGUguaggcu, AAAguaagcg, CCUguaagcc, AGGgugauuu, UGUguaugaa, CUGguacaca, AGGguagaga, AUAguaagca, AGAguaugua, UUGgucagca, CAGgcaaguu, AAGguauaua, AAGgucugga, CAGguacgca, AGGgugcggg, AUGguaagug, AAAgugauga, UGCgugagua, AGAguaggga, UGUguaggua, UAGguaggau, UAAgugagug, GCUguaagua, GAAguaagaa, UCGgugaggc, UAGguauuuu, AAGguacaca, AAGguaggua, UGGguagguu, ACAgcaagua, GAGguaggag, UGGgugaguu, GCGgugagau, CCUguagguu, CAGgugugua, CUGguaagcc, AAGgugauuc, CAGguagcua, GUUguaagug, AUGguaagca, AUAguaggga, GGGguucgcu, CCGgucagag, GUAguaugag, CGUguaagau, UGAguaggca, UCAguaugua, GAGguaucug, AGAguauuuu, AAGguuguag, AGUguaaguu, CGGguaaguu, UCGgugcgga, UAGguaagua, GAAguuagau, GCUgugagac, CAGgcaggua, CAGguagggg, UAAguuaaga, AUGguggguu, UAGguaaguu, CUGguaaauu, CCGguaagga, GAGgcaggca, CAUguaagug, AAGgugccua, UUGguaggga, AAGguaaaca, CGGgugugag, GGGgugugag, UCCguggguc, ACGguaaauc, UCAguaggua, CAGgucagcc, CAGgcggugg, CGAguaagcu, CCCgugagca, AAAguaauga, CUGguaagcu, CGGguaacca, CAGgucgcac, GAGguaggcc, UAGgugagcc, UAGguaggca, GCGgugcgug, AUGgugagua, GGGgugaggg, GAGgucacac, CAGguaggcc, CAAgugcuga, GUCgucuuca, CAUguaagaa, GUAguaagga, UAGguuugua, CAAguuagag, AAGguagagu, AAGgugagau, AAAguaggua, ACAgugaauc, CAGgugugcg, CAGgucggcc, AAGguaguau, ACUgucaguc, UCUgcagccu, CGAguaagug, AGAguaauua, AGUgugagug, CCGgugagcg, AAGguaaccu, AAGguugugg, AAGgcauggg, AAGgucagag, ACGguaaggu, GGGgugagca, GAGguugcuu, AAGguaucgc, CCGguaaagg, AAAguuaaug, UAGguacgag, ACCguaauua, GGGguaagga, CCGguaacgc, CAGgucagaa, AAGguacuga, GAGgugacca, GGGgugagcc, AAGguacagg, AUGguaauua, CAGgugagag, AAGgugacuc, AUAguaagua, GAGguaaacc, CAGgugggau, CAGgugagaa, AGGguaaaaa, GAGgugugac, CACguaagcu, CAGguccccc, CAGgucaggu, CGGguaaguc, ACGguauggg, GAUguaaguu, CAAguaauau, CAGguugggg, CCUgugcugg, AAGguaugau, AGGguagagg, AAGguggguu, CAGgugugaa, UUGguaugug, UUGguaucuc, GGGgugagug, CUGgugugug, AGGguagggc, GUGgugagua, CAGguaugua, AAGguacauu, UUAguaagug, AAUguauauc, CUUguaagua, GAGguuagua, CAGguaaggu, CAGguaaugu, AGGgugaggc, CAGguauuuc, CAGgucugga, GGGgugugcu, UAGgugagug, AAUguaaccu, UAAgugaguc, CAGgugcacu, ACGguaagua, GAGguauccu, UCUguaaguc, CAGguauuca, UGUguaagug, CCAgcaaggc, GAGgugaagg, AAUguggggu, UCGgugcgug, UUGguaaggc, GAGguaagug, AAAguaagau, UAGgucuuuu, GAGgucugau, CCAguuagag, UGGgugaaaa, AGAguaagau, CAGguaauug, CAGgccgguc, CCGguaagag, GAGgugagcu, CUGguaagac, CAGgugagau, CUGguuuguu, UGGguaggua, CAGguuagug, CAGguguucg, CGGguagguc, GUGguacaua, AAGguacuaa, GAUgugagua, UGUguaagac, GAGguagccg, UAGgugaucu, CAGguacgug, CUUgucaguc, GAGguaucac, GAGguaauga, AAGguaacac, CAGguaaagc, AAGgcaagua, CGCgugagcc, AGUgugcguu, GAUguaagca, AAGguaauag, GGAgcaguug, AGCguaagau, AAGgucaggc, GAGguauuca, AAUguaaagu, CAGguaacaa, UCGguaggug, AAAguaaguc, CGGgugcagu, GGUgugugca, UGAgugagaa, CACguguaag, GUGguuggua, GCAgccuuga, CGAgugugau, CAGguauaua, UAUguaugug, CCCgugguca, AUGguaagac, GAGgugugga, AGUguauccu, UGAguguguc, UGGguaaucu, AUGgcagguu, GAGguaagau, UCAgcagcgu, AAGgugggau, CGGgugcgcu, CAGgugucug, AGCgugguaa, AAUgugaaug, UCGgugagac, UAGguaaagc, CUGguaaaag, CCGgugcgga, CAGguacuca, CAGguagcaa, GAAguugagu, GAGguggagg, AGGguaugag, UAGguaugcu, UAGgugagac, CAGguaauua, CGUguaagcc, CUUguaaguu, AAGguaacuu, UCGgcaaggc, GAGguucucg, GAGgugggcg, AAGgcaugug, CUGguauguu, UAAgucauuu, CAUguaauua, AAUguaaaga, UAGgugcuca, AAGguaaugg, GAGguacuga, UGGguaagua, UGGguaaaaa, AAGgugagcu, UACgugaguu, AGGgugagcc, CGGgugagga, UGGgugagag, GGUguaagcu, CGGguggguu, CCAgcuaagu, AAGguuuguc, GAGguuagac, GAGguaccuc, UUUguaaguu, GAGguuagga, CAGguaggga, AGGguaauac, UGCgugugua, CCAguaacca, AGGgucuguc, UGGguaugua, GUGguaagcu, CAGguaaccu, AAGgugaguu, UAGguucgug, AAAguuagua, UGGgcaaguc, AAGgcacagu, GUUguaaguc, AAGguuugcc, CUUgcauggg, GCGgugagua, GGGguaagcg, GCCguaagaa, GAGgucggga, UUGguauugu, AGUgugagac, CUGgugggga, AGAguaaggu, CCGguggguc, CAGguauucu, UGGguaacgu, UUGgugagag, UAGguacccu, GGGgugcguc, AAGgcaggag, ACGguacauu, GAGguaguua, CAGguauggg, UUUguguguc, CAGguacuua, AUGguauacu, AGUgugagcc, ACAguaacga, CUGguaccca, CAGguaaccc, GGAguaagua, GAGgugggug, ACUguauguc, ACGgugagua, CUGguaaugu, AAGguaucag, CAGgugcccc, AGUgucagug, AAGguaggag, GGAguaugug, UUGguauuuu, CCUguuguga, UUUguaagaa, UAGguaacau, CAGguaagca, CAGgucacag, CAGgugugag, UAGguuugcg, CUGguaagaa, ACGguuguau, AAGguugggg, AAGgugaauu, GGGguuaguu, ACGguaaggc, CAGguuuaag, CUGguaaguu, GGGgugagag, UGGguggguu, GAGguuuguu, UGGguaaaug, CAGgcaggcc, CACgugcagg, AAGgugagcc, CAAguaagug, CAGgucaguc, GCGguauaau, UAGguaaagu, UAGguggauu, GAGgucugga, UCGgucaguu, UGGguaacug, AAGguuugau, UGUgcuggug, UGUguaccuc, UGGguacagu, AUCgucagcg, CAGgucuugg, GAAguuggua, GAAguaaaga, UUGguaagcu, UAGguaccag, AGGguaucau, CAGguaaaaa, ACGguaauuu, AUUguaaguu, GAGguacagu, CAGgugaaag, UGGguuguuu, GGGguaggug, CAGgugccca, AGCgugagau, CCAgugagug, AGGguagaug, UGGguguguc, AUCgcgugag, AGGguaagcc, AGGguagcag, UUCguuuccg, AAGguaagcg, UGGguaagcc, CAGguauggc, UGUguaagua, AAGguagaga, ACGguaauaa, CUGguacggu, GAGgucacag, UAUguaaguu, CUGguacgcc, CAAguaagau, CUAgugagua, CCGguaaccg, CUUguaaguc, GUGgugagaa, ACCguaugua, GUAguaagug, UUGgugggua, CGGguacuuu, UGGguaaaua, AGAgugagua, AAGguagguu, AAGguaugcg, CCUguaggcu, ACAguagaaa, CCGguuagua, CGGguaggcg, GCAgugagug, GAGgugaguc, CUGguagccu, CAUguaugua, GAAguaacuu, GAAguaagau, AAGguuagau, AAGguaauca, AAUguaugua, UGAguaagau, AGAgugagca, GUAguucuau, GAGguaauca, UAGguaugga, UAGgugggac, GAGguacaug, UGGguaaggc, CAGguacgcc, CCAguuacgc, ACUgugguga, GAGguaaguc, AUUguaggug, ACCgucagug, AAUgugaggg, ACUgugagug, UGGguguggu, AAGguuggga, AAGguuugga, UCCgugagug, CGGgugagug, AGAguaagcu, CAGgcaagcu, UAGguauauu, AAAguagcag, GAGguaaccu, AAGgugggca, AGGgugagua, UGGguaaggu, CUUgucagug, UAGgugcgcu, GAGgcaaauu, AGGguaccuc, CAAgugcgua, AGAguaagac, GUGguaaaua, GAUguaagcg, GAGguaaagc, UAGgugagua, CAGguaacau, CCUguacggc, UAGguauguc, UAGguccaua, GAGgugaaaa, AAAguacuga, UUGguaagcg, CAGgcaagcg, UUUgcagguu, CAGguuuaua, CUGguaaagc, AUGgugagcu, CAGgugguug, GUAguaaguu, CAGguaauac, CAGgcaaggc, AAGguaauuu, UUUguccgug, GAGguagguu, ACCgugagug, CAAguaagcu, ACAgugagua, UUGgugagau, AAGguagucu, CAGguaaagg, GGGguaugga, UUUguaagug, GUGguaagag, AGUgugaguu, AAGgcaagcg, UAAgugagua, AGGgugagug, AGUguacgug, AGGgugcgua, GGCgugagcc, CGAguuauga, CAGguaaaga, UUGgugaaga, AGGguaaugg, AAGguccaga, AGUgugaguc, CAGguaauuu, CAGguaacgc, CUGguacacu, CUGguuagug, CAGguacuug, CACguaagua, GUGgugcggc, GAGgucaguu, AUGguaugcc, AAGgugugug, CUGguggguc, CAGgugaggc, AAGguuaguc, AAGguagcug, GAGgucagga, GUUguaggua, UGGguacaag, AUGguaggug, GAGguaagcc, AUGgcaagua, AAGguauauu, GCGgugagag, AAGgugcuuc, UAGguacauc, ACUgugguaa, GAGguaggcu, GAGguaugca, AGGguaguuc, CAGguauccu, AGGguaaguc, AGGgucaguu, CAGguuggga, CAGguggaua, GGAguagguu, GAGguaggau, GGGguuugug, UAGguaauug, AAGguaaccc, ACGguaagaa, GAGguagggg, CGAguaggug, UCCguaagug, UCGguacagg, CAAguaagcg, AAGguccgcg, AAUgugagua, CAGgugaaug, GUGguaaggc, AGAgugagug, UCUguauguc, UGGgugaguc, UCGguuagua, GAUguaugca, GAGguuggug, GAGguggggc, UGGgucaguc, GCAgugagua, CAGguugcuu, AGGguagagu, UAGgucaggu, CGCguaugua, GAGguauuaa, CAGguaaacu, AAAguaaguu, GGGgucuggc, GCUguggggu, UUGguaaguc, AAGguagaag, AAUgugaguc, AAGgucagcu, AAGguaagag, AUGgugagga, AAGguacuuc, AAGguaagaa, CCGguacagc, GCGgugcgga, CAGguacaua, CUGgugagga, CUGguaggug, AACguagguu, AUGgugugug, UUGguacuau, CAGgucggug, CAGgcauggg, AUGguaucuu, AAGguaacua, CAGgugggcg, CACgugagga, AAGgugguuc, UGGgcauucu, AUGguaagcc, AGGgucagug, AGAguacgua, AAGguaggca, AAGguauuca, CAGguagauu, GAGguauuua, GAGgucuaca, GUUguagguc, CAGguacucg, GUCguauguu, AAGguacuuu, AGAgugagau, AGUguuggua, AAUgugagug, AAGguagauu, AUGguuugua, GAGgccccag, AUGgucaguu, UCUguaagga, CAGgucgggc, CAGguaagcc, UAGgucagug, AGAguaggaa, CUGguacuuc, CUCguaagca, CAGguaacua, CAGguggcug, UGGguccgua, GAGguugugc, CAGgugcgcg, AAAguauggc, UGAguacgua, CUGguacgga, CAAgugaccu, AAGgugaugu, AAGgucugca, AAAguuugua, AAGgugagca, GAUguaagcc, CAAguaauuu, CAGgugugug, UGGgugaggg, AAGgugaccu, UAGgugugag, CAGgcagguc, UCAguaaguu, UCAgcaguga, AAGguaccac, UAAguaggug, AAGgucagcc, CAGguaacuc, AAAguaagag, AAGguagaua, AAGgcaaggg, CAGgugucgg, CAGguggcua, GAGguugcca, CAGgccgugg, UUGguauaug, GAGguugagu, GAGguagguc, GUGguaagac, UAGguccuuc, GAGgcaaguc, GAGguaacau, CAGguauauc, UCGguugguu, CAGgugaacc, CAGgucuuuu, CAGgcauggc, AAAguacuug, CAGgugauuc, UUGguagguu, UAUgugagca, CAGgugagcg, AAUguaauaa, AAAguaaggc, UAGguuuguc, UAGgugggag, GAGguaaguu, AAGguagccg, CAGguggugc, UGAgucaguu, CUGguaggcc, CAAguaagga, CGGguaaggc, AAGgcgagga, CAGguaguuc, CAGguaagga, CCUgugagug, AAGguaaaug, CCGguaauua, CAGguaaguu, AAGgugguca, CAGguaccuc, AUCguaagua, CCGguacaua, GCGgugagug, GAGgugguau, CUGgugugga, GAGguaauuc, CAAguacgua, UCUguaagug, AAUguaagug, AGGgucuguu, GAGguacugc, AGGguaaggc, AAGgcaagag, CAGguggguu, UAGguuagga, UGAguaagcu, AGAguaagag, AUGgcaggug, UAGgcaagua, AUGguaggua, GCAgcccgca, ACGguaaacu, AGGgugaguu, GUAguagucu, GUGgcugaaa, CAGguuaguc, CUGgugagca, UCAguaagug, AAAgugauug, UAGgucugga, GAGguguuuc, AAGguaaauu, CAUguacauc, AAGguuugaa, CCAgcaagug, UAGguaauaa, GAGgcaagug, CAAgugauuc, CAGgucgugg, GAAguaugcc, UCGgugcccu, GAGgucaguc, CAGgugagac, UUUgucugua, CAGguagaua, UGGguaucag, UAGgugggcu, AUGgugagau, CAGguaacac, CCGguauccu, UAGguaagcu, UCAguacauc, UAGguuugcc, AUGguaagaa, UUGguaagac, CCGguuaguc, GAGguaagaa, UGGguaaguu, CCGgugagaa, CCUgugaggg, ACGguaggag, ACAguauguc, CAGguauuaa, CAGguggauc, AGAgugcgua, AAGgugaccg, AGAguaggug, ACUguaugua, UAGgucaauu, AGUguguaag, CGGguaccuu, CUAgugaguu, CUAguaagug, CAGguacaac, UAGgugugug, CAUguacggc, AUGgugugag, AGGguggaag, CAGgugcgag, UAGgugcucc, AAGguggugg, AAGgucuguu, CAGgugggcc, AAGgucaguc, CAGguuuuua, AACgugaggu, CGGguaagag, UUUgucggua, UAGguuaagu, GUGguaagaa, CAGguauugg, GCUguaaguu, CUAguaagua, UCGguaaaua, CAGguaacuu, CCUgugagua, CAGguuauau, CUGgugaaca, AAGguauaaa, GAGguaagca, AAGgugaagc, CAGgugaguu, UUUgugagua, CUUguacgcc, AGAguaagug, UGGguaggug, UGAgcccuge, UGUguaugua, AAGguagagg, GAGguggggg, UAGguaauuc, AAGgcauggu, AGAguaagca, AAGguaggaa, CAAguaagua, ACUguaauug, CAGgucugug, UCGguaccga, CUGgugagag, AAGguuugcu, AUGguaccac, UAAguuaguu, CAGguaggac, AGAgugaggc, CGAgucagua, CAGgucugag, GAGguggugg, ACGguauugg, GCUgcgagua, CUGguaagug, GUGgugagau, GGGguuugau, UCUgugagug, CUUgucagua, GAGguaaaac, UCUguaagau, CCAguaaguu, CAGguaaagu, GCGgugagca, UAAguaagag, CUGgcaggug, GAGguaaggg, UGAguaaguu, GAGgugagac, GCUgucuguu, AAGguaacaa, GAGguaacgg, CUGguauucu, CAAguaacug, AAGguggggu, UAGguauggc, CAGguauuuu, GUGguaaacu, GAGgucugag, CUGguaaggu, CAAguaaguu, AAGguagacc, GAGgcgagcg, CUGguaaaua, UGUguaagcg, CAGguuaggg, GGGgugagga, ACAguaugug, CCGgugggga, GAGgucagug, AGGguaaggu, ACAguaagua, GGUguaaggu, GAGguaauaa, CAGguauucc, CUGguauaaa, CCGgucugug, CAGguaacug, GCAguaagua, AAGguagggg, CAAguccacc, CAAguuggug, CAGgugcggu, CAGguaaaau, ACGguaagga, UGGguaauaa, UAGguaagug, CCGguagguu, AGAguaugga, CUCgugaguc, AAAgccggug, UUGguaauuu, GAGguaaaag, CCUgugugag, AAAguaagga, UGAgugagug, AAGguacaug, CCGguaaaug, CAGgugaagc, CAGguacccg, GAGguaaggc, UUUguauguu, CAGgugcucc, UCGguagguc, CGGgugaggc, AAGguaauua, ACUgugaguc, AAGgucagca, GUGgugagug, CAUguccacc, AAGgugaccc, CGGguuagua, GCGguaguaa, GCUguaggua, CCUguugagu, UAGgucuggc, GAUgugagcc, CUUgugagua, CUGguguguu, GAGgcaugug, CAGgcaagag, UUGguaagaa, GAGguguggg, GAGguauuuu, CAGguaguaa, AGGguaagac, UUUguaggca, AGGgugagau, GAGguuugua, AAGgugagug, GAGgugggag, AAGgugagaa, CUGguaagag, AUAguaaaga, GAUgugaguc, AAGgugcagg, CAGgucuguc, GAGgugauuu, CAGguuggcu, CGGguauggg, AUGguccauc, CCGguuggug, GGAguaaguc, AAUguaagga, CAGguuuguu, UAGgugugua, UAUgucuuug, ACGguacuuc, AAGgcacgcg, CUGguaaacc, CUUgugggua, UGAguaaguc, CUGgugggug, GAGguggaga, GUGguggcug, GUGguaagug, AACgugagua, GAAgcuguaa, CGGguaucuu, CAGgugucag, AAUguacgca, CCGgugggua, UGGgugaggu, AAGguauguu, CAGguauguu, CAGguuugcu, UUGguaaguu, CAGguaguug, CCUgugaaua, GCUgugugug, CAAguaauuc, AGGguaaugu, GCUgugaguc, ACCguaaguu, CGUguaagua, GGGguaaguc, AAUguaugau, AAUgugauua, UCAguaagaa, CAGguccguc, GAAguauuga, UUGguaagga, CAGgucgguu, UAGguuagug, ACGguaaaac, AAGguagguc, UACgugagua, UUGguaagca, GCGgugaguc, GAAguaaggg, CGCgugaguu, CAGguacccc, UCUguaagac, GAGgugggca, AAUguaagac, CAGgcaaggg, CAAguaacua, AAAguuuguc, CAGguacugu, AAGgucccuc, UCGguaaguc, UGGgugagug, CUUgugagau, AGAgugagcu, UAAgugggga, UAGguaggga, CAGguuagcc, AGGguaauca, AAGguucagc, UGGgugggug, CAGguuguga, AAGguaagug, CAUgugcgua, CCGguauauu, ACCguaugug, CAGguauagu, CAGguauuac, CAGgugcagg, GUGgugagcu, AAGguaacau, CUGgugaugg, AUGguaaaug, CCGgugagca, AAGguaaacc, AAGguacugg, GCGgucagga, CUGgucaggg, AAAguacguu, AGAguagguu, AGGguaagcu, AUUgugagua, CCGgccacca, GAGguaacuu, GAGguaugaa, CAGgucagac, UAGgcgugug, AGGguaaguu, CAGgcaugag, CAGguaacgu, CAGgcgagca, UAGguauggu, AGAguaggau, CUGguuucaa, GAGguaaacu, CAGgcaugca, UUGguaaucu, AGGgcagaau, AUGguaaaac, GCUgcaggug, GAAgcacgug, CAUguaaaca, UGGguaagau, AGGguagcua, AGGguggggu, CCUguaaguu, UGAgugaguu, GGAguaugua, CAGgugaccu, AAAguacgga, GAGguacaga, GAUguaggua, GGGguaauug, UAGguggguu, GUGguacgua, AAGguacagc, GAGgugaaga, GGGguaagca, UGAguagguc, GGGguaaguu, AUUgugaguu, UCAguaagac, AGUgugagcu, AAGgcaaaac, CUGgugaguc, AAGgucucug, GAGgcugugc, AGAgugagac, GAGgugaugu, AGAguauggu, UGGguggguc, GCUgcugagc, CAGguagcug, UAGgucagaa, CCGguaggug, GCAguaugau, CAGguuucag, GAGguuugcc, GGGguggggg, AAGguacaua, UGGguguguu, AGAguaaggc, GCGguuagug, AAGgugacuu, AUGguaagau, AUGguaguug, CAUguaagac, CUGguaugua, UUCguaagga, GAAguaugac, CGGguaauuc, UGGguaacuu, CAGgugccua, CAUguagggc, ACCgucagga, CGUguucgau, GAGgcaggac, UAGguaauau, UCGguauacu, UAGguugugc, CCGgugaguc, CAGgugccaa, CAGgugaugc, AAGgugagga, GUGgugaggg, UGGgucagua, GAGgucaggg, UAGguacgua, GAGgcaagag, CCUguuggua, GAGguaucca, UAAguaagcu, AAGgucaguu, AAAguuaaag, GAGgugcuau, ACGguaaguu, CUGgugaggg, GAGguuaugu, CUUgugugca, UGAgcugggg, AAGguauagu, UAGguaaaac, GGGgugaggu, GAGgcaagca, GGAguaacgu, AGAguaagua, AAAguaagua, GAGgcaacca, UGUguaaguu, UAGgugaggc, ACAguaagaa, UGAguaagug, CAAgucagua, AGGguaaaug, AAGguaugca, GCUgugcgug, GAGguucgcc, AAGgcuugca, CAGgcaagug, AUAguaaguc, UUGguaggua, GCAgcaggua, AAGguauauc, AGCguaagcc, CUGguucgaa, ACGgugggug, CUGgucauug, CAGgucagga, CAAgugagac, GAGguacugg, GAGguguagu, GAGguguccu, CAGgugcgua, AGUgcccuga, AUGgugaguc, UGUgugugua, CAGguaugcu, CUGguacagu, UUGguacgua, UCUguacgua, UAAguaauuc, CACguaugug, CAGgcaagua, UCGgugagug, GGUgugaguc, UCUguaagcu, AAGguucaga, AGGguacuuc, GCGgcagguu, GAGgcccgug, CAGguauaaa, AUGgucaagu, AAGgugagua, GUGguuuguu, AGAgugagga, GAGguaugac, UAGgcgugag, AAGguacucc, UGAgugagga, GAGguaugau, GGGgucggua, ACGguaugca, CAGguaccac, UAAguaccug, AGGgugggcu, CUGgucuguu, UAGgucagag, AAGguguguu, CUGgucagug, AAGgugggac, GUGguaguag, CUAguuuagg, CCCgccccau, GCUguacugc, GAGguaauau, UAGguuggug, AAGguccaac, UAGgugagga, GUGguaaguu, AGUgugagag, AAUguacaug, UUGgcaggug, UAGguuauug, CAGguacuga, GCGguggguc, UGUguaagau, GAGgugagua, GCAgccccgg, CAGgugcuaa, AGUguaagag, CAGguacauc, CAGgugggac, AGGguaaaua, UAAguaauua, CAGguaaccg, AAGguuugca, UAGgugguuu, CAGgugaccg, UGUguaagcu, GGAgugaguc, AGGguaggag, AGGgugggug, AAGgucugag, GAUguaauau, GGGguaauua, UAGguaggua, GAGgcaagua, GAGguaagga, UAGguacuac, UCGgugggug, AAGgugugga, CAGgucugcc, UAAgugagcc, GAAguaaguu, GAAguaagcc, UAGgugcgac, GAGguauggc, GCAguaagaa, CAGgugugga, UUGguaacgu, GCUguaaaaa, UUGguuagua, AUAguaaggg, UUGguacuag, CGGgcagccg, CAGgugcugg, UAUgugaguu, CAGgucuggg, UAAguaagaa, AAGguuauua, AGAguaaagc, AGAgugugag, UAGgugcgag, CAAguaaacg, AAGguacgua, CUGgugagua, CCAguaugua, UUGgugagug, UGAguaagua, GAGguuagca, GUGguaagcc, CUGguauggc, AAAguaacac, CAGguacuaa, UCUguaaguu, GAGgugaggg, ACUgugggua, GAUguuugug, CAGgugucaa, CAGgucacca, CCGgugagua, UUGguaaaua, CAGguggggg, ACUgcaggug, UAGguauguu, GGAgcaagug, UCGgugccuc, CAAguaacuu, GAGguaacca, CAGguaauau, GGAguaagaa, GAGguaccuu, AGGguaagga, CCUgugaguc, GAGguaaugg, AUGguguguc, GGGgugagua, AGGgucaggu, UGGguaaggg, AGGguagguu, AUAgugaguu, CCCguaggcu, ACAguaugua, GACgugugua, GCGgugagga, CAGgugaccc, UAAguuuagu, ACAguugagu, CGGgugaggg, CAGguggauu, CGGguagagg, UAGgugcgug, GGGguaagaa, GAGguggggu, CACguggguu, ACGguaauug, AGAgugaguc, UUGgcuccaa, AAGgugaugc, AAGguugguc, AGCguaaguu, AUUguaugua, UCAguuaagu, CAAguacgug, CAGgugcgug, CAGguaggua, AUGguggggu, AUGgugaguu, CAGguaauca, AAGguagggu, CAGgccaagg, GUGgugagag, AAGguuggug, CAGguacucu, UAGgcaugug, UUGguaccuu, CUGgugugcc, ACAguugcca, UUGguaauau, GAGgugcaug, UUGguuugua, UUGguaagug, UGUgugugug, GUGguuugua, GCGguacaca, AGAguaugcu, UUUguaagua, UCUgugcggg, AAGgucagug, GAGguaggaa, GCGguuagca, AGGgugaggg, GAAgugagua, CAGgugacag, AAGgugauua, GAGgccagcc, GAGgucuccu, UAGguauuac, CAUguaagag, CUGguagggc, GAAguaagua, CGGguaagug, CAGguaaucu, GUGguaggua, CAGgugggua, AAGgccagug, AAAgugaauc, ACGguuacgu, AUGguaggaa, CGGgugagac, GAGguuggaa, UGGgugagcc, CCAgugagua, CUAguacgag, CAGguaugac, GCUgugaggu, CUGguaugaa, GGUguacgac, CUUgugagug, GUGgugagca, CUGguaacuu, CAGguacuau, AGGguaaggg, UUGguuaguu, GGUguaagca, UCGgugagga, UGGguaaaca, UCGguacgug, UAGguagcag, CUGguaaggc, GUGguaagga, UAAguaagca, GAGguuccaa, CUGguaugga, GGGgugggua, CAGguuuccc, CAGgucucug, GAGgugagga, CUUguggguu, AUGgugagac, CAGgugaagg, GCGguagggg, GUUguuuccc, AAAgcaucca, GUGguagguu, AAGgugugaa, CAGguacagu, AAGguaccaa, UUGguaauug, AAGgugcuca, AAGguucaac, CAGguuuaca, GCUguaagug, AGGguauguc, GAGgucgggg, AAGgugccug, AAGguaaaaa, GUGgugaguu, UAGguaagaa, AGGguauccu, GUGguaauau, UCUguaagua, UGGguaugga, AUGguaugga, GACgugagcc, CUGguuuggc, AUGguauauc, AAAguaaacu, AGCgugagug, CUGguauaga, CAGgugggga, AGAguauguu, UAGguacuug, GCAguaggug, AGUguauguc, AAGguuaagc, CUGguggccu, GAAgugaguc, UUGguguaag, CAGguaagaa, CGGgucucgg, GAGgugcaca, CUCguuaguu, AAGgugauca, UAUguaagaa, GAGgugcuug, CAGgugguca, ACGguaaguc, ACAguaaugu, CCUguaaggu, GAGguuaagu, UCGguaugug, UGGguauguu, AAGguauuac, CAGgugaggg, UUGguaaaca, AAGguagugu, GAGguguggc, CAGguacgga, AAGgucauca, CAAguaggca, CAGgugaaac, CAGguacugc, AAUgcaagug, CAUguaauuc, AAGguaugcu, CUGgugaguu, CAGgugguuu, UGUgugagua, AAGgucggug, AUGguaaauu, AGGguauuac, AGUguaugga, AACguaagau, GUGguaaggu, ACUguuagua, CAGguaucag, AAGguuaguu, CUGgugagcu, UUGgugagcu, UGUguacgua, GAGgucagcc, GAGguagaau, AAGguaugag, UAGguauuuc, UGUguaacac, AGUguaaggc, GAGgucugcu, AAGguuagca, CAGguaaaug, AACguaagcu, CAGgucugca, CAGguauugu, GUGguaauuc, GAGguauaug, GCCgugagcc, GAGguaagag, UGAguaugua, CAGguaaggg, GAGguaaauu, CAGgcaacuu, UGUguaaguc, CAGgugcgcu, CGGguaaacc, CCGgucaguc, UAGgugggcg, GCGgucaguu, GGGguggguc, AGCguaauag, ACGgugaguc, CUGguacuug, CAGguuggua, AGAguaugug, CUGgugggua, GAGguggcuu, AUAguauuga, UGAgucgucc, CAGgugcucu, UACguaauau, GCUguccuga, CAGgcugcac, CUGgugcgcu, GCGguaagaa, UAAguuacuu, GAAgugagug, UAGgcaaguc, UAAguaaaua, ACGgugagug, CAGguagguu, GGGguauaac, GUUgugaguu, CAUgugagua, GAGgugcauu, AAGguuugua, UCGguaaugu, CGAguaaggg, GAGgcacgga, AGGgugugga, CAGguauggu, AAGguagaaa, CAGgugccug, UGGguauaug, UGAgugagac, UGGguaauuu, AUGguaaaua, AAGgcaaagg, AGUguuuguu, AUGguauugg, CUGgugaggc, UUGguaaaau, ACAgugaguu, CAGgugcugu, GAGguuaaga, AGAguaagaa, GAGguccgcg, GUGgugagga, CAGgugagcc, CAGgugacau, AUGgcaagcu, UCGguaauau, CAGgcaacaa, GGGguaggga, CUGgucucgc, UAGguaacga, CGGguaaggu, UAGguaaugc, CAGgcaagaa, ACAguaggua, CAAguaugag, GCUguucgaa, AAGguuaugc, GAUgugaguu, CAGguggaga, AGAguuaguu, UGAgugugcg, GAGguacagc, CAGguaagac, CAUgugcuuu, AGGguguguu, ACAguuaagg, ACAgugaggg, GAUguauacc, UUAguaagcu, CAGguaagau, AGAgcugcgu, GAGgcaaguu, GAAguaagug, AAGgugaaaa, AAGguaccua, GAGguaucag, AUGguaugua, AAGguaugaa, UUGgugagcc, AAGguuagga, AGGguaugua, CAGguaccga, AGAguaaacu, AAGgugcaua, AAGguaaugu, CCGgugugug, AGGguaaauu, GGGguuuggc, CAGguacacg, UUGguaacca, GAGgucaggu, UCUguuggua, CAGguuaguu, UUGguauguc, AAGgugcguc, AGGguaagaa, UUUguaagcc, AAGgucaggu, CUGguaaacu, UCGguaauuu, CUGguaggcu, GAGgucugua, GAGguacuuu, CUGguaaagg, CGGgugugug, CAGguguggu, UCGguacguc, CAGgugccag, GGGgugagaa, ACAgcuagua, AAGguauagc, CUGguaggag, GCUguacgua, AAGguaaagg, CAAgcacgag, CUAguaagac, CCCguaagcg, CAAgugugag, AUGguaaggg, AAGgugaggg, CAAguaggua, GGUguugcug, GAGguacugu, UAGguaagau, CAGgugcgaa, GAGguccagg, UUGguauaca, GGAgugagua, GAGgugagau, AAGguggggc, CAGguaaacg, UCGguaacuu, CAGguaaauu, GAGgugcgca, ACUgugagua, ACGgugugac, GUGguaaguc, CAGguaggca, CAGgucagca, GUGguaugug, AAAguaucug, CGGguaugua, AAGguaauaa, GAGgugggga, GCUguaggug, GAAgugaguu, AAAguauuua, UAUguaagua, ACGguaugag, CUGgugagug, AGAguaaaau, GCUguauggc, AUGguaaacc, GCAguaauaa, UAAguauuua, AAUgucagug, AUUgcaggag, CCGguaagaa, AAGgcaaguu, GAGguuuguc, AAGguaacug, AAAguaugag, GAUguuagua, CAGguggguc, AAGguaccga, CCAguaauua, GUGguaugcg, AUGgugcgcu, CAGgucuaug, AAGguauuua, CUAguaagau, AGAguaauuu, GAGguaacgu, AAGguagcca, CUGgucccgg, GAGguccuuc, ACGgucaccc, AAGguaauac, CAGgugcaug, AUGguaauag, UUUguaacac, UGGguaugau, CAGgcccccc, AGAguaguaa, AGUguaagaa, GAAguauguu, CAGgugugca, UUGgugaggg, UGGguugguu, CAGguacgua, GAGgugcggc, UCUguacggg, CGGgugcgug, UACguaagug, CAUguaagga, CAGgugacgg, GAUguaugcu, UCUgcaauuc, UGAguaaggc, GAGguauauu, AGAgugaguu, AAGguaagcu, UAGgugaagu, CAGguuagua, UAUguaagug, UUGguggggg, UGAgcucaaa, UCGguaugua, UAAguaugcc, AAUguaagua, CAGguuugca, ACGgugagag, CAGguguuuu, GUGgugagcc, AGGguacaua, UAGguaaccc, GUGgucagua, CUGgugagcc, CAGgugcuua, AUAgucguga, AUAgugagug, GAGgucaaaa, CGUguagcuu, CAGguguuug, CAGguuggac, CAGguaagcu, AGGgucagaa, CACguauguc, CACgugagug, GGGguacgga, AAGgcaggac, GAGgugaagc, GAGguuugaa, CAGguaagug, CAGguaacca, CAGguacucc, AAGgugcuuu, GAGguaaaua, GAGgcaggug, GAGguucgga, CAGguauuug, CAGguaaaua, CAGgugaugu, CAGgugauac, GAGgugaggc, AGGguggggg, UAAguaaguu, UGGgugaaca, UAGguacugc, CAGgcuccug, AGGguaggca, CAGgugcccg, GAGguacauc, AGGgugugug, AAGguaguaa, UGGguaugag, GGGgugugug, CUAguaggug, GAGgcaagga, AAGgcaagac, AAAgugcggu, AAGguugguu, GAGguuaaug, UUGgugaguc, UCGguuagcu, GCAguaagca, AAGgcaagca, ACAguaagcu, GAGguaacag, AAAguacgua, GAGguaauac, UUGguaggug, CUGguuaguc, GAGgugacgc, ACAguaagga, AAUguacuua, GGGguacagu, CGUguaugug, UCCguagguu, GAGguggucg, UCAgugaguc, AAAguaagca, GAGgucuggu, GAGguaauua, GUAguaagua, AAGgugggga, UCUgugagca, GAAguucgug, ACGgugaggc, UCAgugagua, UAGguaguug, GGUgucuggg, GGGguaagug, GAGguggguu, UGUgugaguu, CAUguaagua, AAGguaggug, AAUguaggag, GAGgcacguc, CAAguacauu, UUGguacaga, GAGguaguag, AAAgugaggg, UUGgucagug, AGGgugaguc, CAGgugaaca, GGUgugggcc, CGGgugagcu, GGGgugaguc, ACAgugagag, AGGgugaggu, GCUguaaguc, AUAguagguu, CAGgcaugug, AAGguaaguu, CAGguccgug, GAGgcaggua, AUGguggaag, AUGgugggcg, GAGgugagaa, AGUgugagca, UUGguaagua, CAAguaagca, GGUgugagcu, CCCgugggua, CAGguagaau, CAGgcugagc, CUGguggccc, UGAguaagag, CACguuagcu, AAGgugaguc, AAGguagcuc, UCGgugaguu, GAGgcccuuc, CAGguuaugc, CCUguaagcu, CAGgucuccu, UAGguaggcu, GGGguagggg, AAGguaguga, GAGguuguug, CAGguugguu, AAAguaagcc, ACAgugagug, UGGgugugau, CCCguaacua, AAGguguugc, AAAgcuggug, GAGguauagu, ACGguaagag, AUGguacggu, GAGgccaguu, GAGguaugcg, UCGgugggag, AAGguggaua, CCAguguggc, AGGguaagug, UCUguagguc, CAGgcaagga, CGGguaauuu, AUUgugaguc, CAGguaaacc, AAGgucaauu, AAGgugaaua, GUCguaagaa, GCGguaaguc, CUGguagage, GAGgucgguc, CAGguaaaca, AAGgcaagga, CAGgucgucu, GGGguagggc, CUGguacuaa, GAGguagcug, CUUgucagcu, UAGguaaggc, CUGguauuac, UAAguacguc, AAGguaagcc, ACGgugaaag, CCAgccaaua, CAGguuuguc, AAGguauaau, AAGgucuuag, AGGgugagcu, AAGguuaggg, CGGguaaauu, CAGguaacgg, AGAgugugua, ACAguaaguu, GAUguaauuu, GAGguaggga, UUGgcaagug, AAAgugagga, AAGguagugc, AGAguaauuc, GGAguaaaua, GUGguaccca, CAGguauugc, GAUgugaggg, CAAguaaauc, CAGgugucuc, AAGguaacag, UUGguaaaag, CAGguaucau, ACGgugagac, CUGguaugac, CAGguucacu, GAGgugauca, AGUguaaguc, AACguaagua, AAAgugagug, GAGguacagg, CAAguaauga, GAUguaagga, UCAguucccc, GCGguaagga, UAGguacuaa, AAGgugaaag, ACUguaagug, UGGguaugug, AUGguaacag, CAGguagggu, ACAguaagug, AAGgugcucc, AAGgugugcu, AAGgugguga, ACGgugcgcc, AAGguauugc, GGGguaugug, CAGgugggcu, GAGguauguu, AACgugaaua, CAGguaaugg, UAGguaugau, CAGgcaggug, GGGguugguc, AAGguauggg, UAAgugaggc, CAAgugaucg, AAAguacggg, AGAgcuacag, GAGgugggaa, CAGguacuuu, GAGgugagag, CAGguagguc, UGGguacagc, AAGgugucag, AAGgcaagaa, GAGguaaaca, AAGguaaagu, AAGguaguca, CUGguauguc, GAGguauggg, AAGguauugu, CUGguacuga, GAGguaagcu, UGGgugggua, CAGguucgug, AAGguauggu, CAGgugagca, UGGguaaauu, UGUguaggug, UGUgugagcc, CUGguaauau, AAAguauguu, UGUguaagaa, CUAgugagaa, AGGguagguc, AAGgugggug, UCGguaagug, AGUguaaaua, GAUguaagug, AAGguuagug, UAGguaagca, CAAgugagaa, AGUguaagua, CAGgugaauc, UGGgugagac, AAGguagggc, CUGguuugug, GCGguagggc, GAGguaaucc, AUUguaauaa, CUGgugaaua, AAGguuuaaa, CCUguacugu, GCGgugagcg, AAGguaaucc, UAUgugagua, CCCgugagug, CAGgugcaga, CAGgucaguu, CAGguaggcu, AAAguaagug, UAGguugguc, CAGguugccu, AAGguaugga, GGUguggacg, AAAgugagaa, AGGgugagag, GAUguggcau, UCGguaaggu, GAGgugcguc, CGGgugaguc, AAGguacggg, GAGguucuug, AAGgugcuug, UAGguaugua, AUGgucagca, CGGguacuca, AGGgugagga, AUCgugagua, UCAguaagua, UAGguaaaua, AAGguaauug, GAAgucagug, CAGguacaaa, AAAguuaauc, AGCgugagcg, CCGgcuggug, AGUguaauuu, UGAgccacuc, GGGgucugua, AUGgcauguc, CGGguaaaga, AGGguagcau, CGGguaggag, GAGguucgug, UAAguuauuc, UAUguaagau, AAGguaguuu, CAGgugguau, GUGguaauga, AAGgugauuu, CAGgugaagu, GUAguaauua, AUGguuggug, CCAguaagug, UAGgugagag, AUGgugaggc, AAAguuagug, AAGgugccuu, UAGguaugag, CAGgugugac, CUGguggguu, AUGguaagga, UCUguaagaa, UCCgugaguu, AAAgcaggua, UAUgugagug, CAGguggagg, CAGguuagac, AUAguaagac, AAGguguugu, GAGgucugug, AAGguaagau, CAUguaaguu, CUGguaauua, CAGguaggcg, AGAguaaguc, UGGgugagga, AAUguaggua, UAGguuagca, GGGguaggua, GAGguauugc, AUUguacaca, GAAguaggua, GGAguaagcu, UAGguaugug, GAGgugaaua, GAGgugggau, AAGguaaucu, GGUgugaguu, AACgugaguu, GAGguaaccg, UAGguaagga, AUUguaagaa, UGGgugagca, AAGguaaggc, CCAguaucgu, CCGgugggug, GAGguagugu, ACGgugggaa, GAGgugaccu, CACguaugua, AGGgugggga, AAUguaaguc, AAAguuaagu, CAUgugagug, AGAguauguc, GCGguaugac, CGGgugaguu, CCGguauuuu, GAGguagaac, UAGguaugaa, CAGgcgcgug, CAAguaaguc, AGUguaagau, AAGguucuac, CCAguaagua, GAGguagcag, CAGgucuguu, CAGguacaau, CCGguaaaga, UAAgugcugu, AGGgugagaa, CUCguaaggu, CAGgucagcu, CAGguaaggc, AGGgugcagg, GAGgugaaac, AGGguaagua, AAUguaugcc, AAGguaagca, ACGguacggu, AAGguaauga, UCUgcucaau, ACGguaaugu, AAGguaguug, ACGguaagug, CAGgugauga, GAGguaacac, GAGguaggua, CAGguaccuu, CAGguaauaa, UUGgugggug, CUGguaauga, UAGguaaguc, AGGgugugac, GAGgcaauaa, GUGguaaagc, CUGgugggcg, GAUguauguu, AGGgugagac, UCGgucagca, AUGgugauua, CGAgugugua, CAGguuggug, AGCgcaagua, UGGguacguu, GAGguauuug, AGUguacaua, AUGguaagua, ACAguagguu, AAGgugagag, UUGgugaagu, AAAguaugua, UGGguaagga, UAGgugccuu, and CCUgugggug.

Additional exemplary gene sequences and splice site sequences (e.g., 5′ splice site sequences) include UCCguaaguu, GUGguaaacg, CGGgugcggu, CAUguacuuc, AGAguaaagg, CGCgugagua, AGAgugggca, AGAguaagcc, AGAguaaaca, GUGguuauga, AGGguaauaa, UGAguaagac, AGAguuuguu, CGGgucugca, CAGguaaguc, AAGguagaau, CAGgucccuc, AGAguaaugg, GAGgucuaag, AGAguagagu, AUGgucagua, GAGgccuggg, AAGguguggc, AGAgugaucu, AAGguaucca, UUCguaagua, UAAgugggug, GCCgugaacg, GAGguugugg, UAUguaugca, UGUguaacaa, AGGguauuag, UGAguauauc, AGAguuugug, GAGgucgcug, GAGgucaucg, ACGguaaagc, UGAguacuug, CGAgucgccg, CUGguacguc, AGGguauugc, GAAgugaaug, CAGaugaguc, UGGguauugg, UGAguaaaga, GUGguuccug, UGAgcaagua, UAUguaagag, AAGgucuugc, AAAgcaugug, AGAguacagu, GUGguaaucc, CAGguagagg, AAGguacaac, UGGgcagcau, CCGgucauca, CCGguuugua, UGAguaaggg, GAAguaugua, GGGguagcuc, GCUguacaua, CUGgucucuu, GUGguaaaug, AUCguaagug, GAGgcaugua, AAGgucuccc, UGGgugcguu, UGUguagguu, GAAgugagca, GGUguaauuu, CUGgugaaau, AUCguaaguc, AGAguaaucc, GGAguagguc, GAGguaccaa, CUUguaggug, AAGguauaag, AGAguuggua, AUGguuugug, UGGgucagau, AGAguaggac, AGAguagugu, AGAguaggag, CAGgucucua, AAGguggaug, UGGguaucaa, GAUguaugga, AAGguguuuc, GCAguguaaa, UUAguaugua, UCUguaugca, AAUguaaaau, AGAguaaauu, GGGguacuuu, GAAguuugau, AAAguagauu, UGUguagagu, UGGguaagcg, CGGguucagg, AGGguacgac, UCGguaagaa, AGGguuggca, AAAguacagu, UAAguuaagg, AUGguaaugu, GUGguuuuac, AGAguaacaa, AAGguagccc, GCGgugaggc, AUGguucagc, AAGguacuua, AAGguccgug, UAGguaagcg, AUGguaccuu, GCCguggugg, CUGgugeguc, CAGguggaaa, AAAgucugua, GAGguaaccc, AGAguauggg, UAUgccccug, AAGgugccag, ACGgugcggc, AGGguacuga, AGAguaagcg, CUGgcaaggg, CCAgugugug, GAGguagacg, CGGgugcggg, GAUguaagcu, AUUguauuua, UGCgugagug, CUGgucuaua, GAGgugcuag, GAGgugccau, CAGguacguc, GAGguucagc, AACguaagaa, AGAguaguac, AAGguaacgg, UAGgugugac, CCGguaauag, CAGguaccag, UUUguaauug, AAUguacgaa, CAGguaauga, AUCgucaagg, CUGguagaug, GGGgugcagu, AGUgugagaa, GGGguuuuau, CCUguccccu, AUUgugaagu, AAGguaaacg, UACgucgugg, AAGgugccau, GGGgucccag, UAUguauggu, CGGguaauua, CGGguacucc, CAGgugacuu, AGUguggguu, AGAguauggc, AAGgccaaca, AAAgcaagua, UCAguagguc, GUGguggcgg, CAUguauccu, UCGgugagcc, AUAguugggu, AAUguuagcu, AUGgugaaug, CGGguaaugu, UCUguaggug, CCGgugaggc, UGAguccacu, CUAguaagag, CGGguggggc, CGAguaagca, UGUgccaauu, UCGguaagcc, UAUguaggug, UUGgugggcc, GAGgcugggc, AGAguaacuu, ACGguagguc, CAGgcccaga, CCGguggguu, AAGgugacgg, GGGguacagc, CAUguaaguc, AUUgugagaa, UGUguaagga, UUUguaagau, AGGgucauuu, UGGguuuguu, CGAguaagcc, GUGgugugua, AUGguauaac, UGGguacgua, AAAguagagu, UCGguaacug, AGAguaauga, AUGguggguc, AGAguaauau, CAGguacugg, UAAgucaguu, GCGguagaga, AAGgugaugg, ACAguauguu, GAUguacguc, UAGguuucuc, GAGgcauggg, AUAgcuaagu, GUAgucugua, AAGgugaacg, GUGguggucg, GAGguugauc, UGAguggguu, ACUguacgug, CUGgugacug, CAAguuaagc, GAGguaccca, AACguaacuu, CAGguuacua, AGAguuaguc, UGGgcacguc, AGUguauggu, AAGguugcaa, CAGguuguua, AAGgcauccc, GAUguaaggc, AGGguacggg, GAGgucaaag, CAAgugagcg, AGAguaaucu, UCGguagcug, AAAguaguag, CAGguucguc, CGUguaugaa, AGUguaaaaa, AAGgucucac, UAGguggagc, UGAguaggug, AGAguaugcc, GAGguugcau, CAAguaagag, UCUgugugcc, GAGgugaugc, GGGgugauaa, CCCgugagcc, AGAguaacug, GCGguaagua, AGAguacauc, UCGgucuggg, UAAguaucuc, GGCguagguu, AGAguacgcc, GAUgucuucu, AGGgcaaggu, CGAguaugau, AUGguagagu, CAAguacgag, UCGguaugau, CCGguguguu, AGGgucugug, GGAguaggcu, AAGgucuaug, GCAgugcgug, UGGgugagaa, AGGguaaagu, GAGguaggac, CUAguaagca, UUAguaggcu, CUGgugggau, CUGguuagua, AAGguacgug, CGGgugagau, AAGgugcaug, AAUgugggcu, CAGguugacu, CAGguuacag, GCGguaacau, AUUgucaguc, CAAguauaca, GAUgucegcc, AAGgugcgga, AACguaagag, UGGguuggua, CAAguguaag, GUGguaacgu, CUGgugauca, AGGguggggc, UCGguaaaga, CAGguacacc, CGGguaaggg, CAAguuugcu, ACAgugcgug, UUGguauggg, GAGgcucauc, CUGguaauag, AUGguggaua, UCAgugaauu, AAUguaauua, GCAgucuaaa, AAGguauucu, GAGgucauca, UGGguccaug, AGAguuugua, AGGguagacu, AAGguaggac, UGUguguuga, UCAguacgug, AUGgucucuc, UGAguuagua, UGAguaaagu, GAGgugaccg, GAGguauauc, CAGgugccau, AGAgugguga, GUUguaagaa, AGAguaaaua, AGGgugaagg, CUGguagauu, GAGguucagg, AGGgucuuca, CUGguaaccu, ACAguacuga, AGAguggguc, AUGguaugag, AAGguuauau, AGAguauagu, AAAguaugaa, UAGguggcua, ACCguauggg, AAAguauaau, UUUguauggc, GGGgucgcgu, GUGgugguuu, CAGguuugac, GGAguaggcg, GAGguacccu, AUGgugugca, GUGguuggug, AAAguaugcu, UAAguuacau, ACAguaugag, GGAguauguu, UUUgugagaa, AAUgugcguu, CAGguagagu, AUGguguuaa, CAUgugcguc, AUAguuggau, GAGguacgua, GUUgugagaa, CAAguacauc, GAGguaguuu, ACUguacaga, CCGguuguga, UGGgucagug, GUAguaagaa, GACguacuuu, AGAgucaguc, UAGguuaguu, AGGgcagcag, AAGguccuac, AAUguaauug, CAGgugcggg, CUGguaaugg, CAAguagccc, GAAgucaguu, ACAguaauug, UUAguuagua, CCUguauuuu, AUCguaagaa, CCAgugagca, GAAguaaggc, UGAgugggua, UCAgugguag, UCUguacagg, CGAgugagug, UCCguaugug, CAUgccguuu, AAAgugacuu, AGAguaggca, GAAguaagag, CAGgcagguu, UUGguagagc, AAGguggaaa, GAGgcagguc, AUGguacgac, AGGguaggaa, AGGguaggua, UUGguaaggu, AUGguacaga, CAGguagagc, UAGguaaggu, GGGguuagag, AAGguaucaa, GAGguagccc, CAGgugccuc, GCAguaagag, ACGguagagu, UGGguaaugg, CUGgucaguu, GUGguacauu, AAAguagguu, AAGgccaaga, CGGgugggca, ACGguccggg, CGAguaugag, CUGguaugcc, GAGguggaug, CAGgccuuuc, AAAguacauc, AAAguaauca, GAGguaacug, CUGguaaaga, CGUguaagca, UGGgcaagua, GCGguggcga, GAGguggccg, AUUgcaugca, ACGgugacug, CAGgucagau, AGAguaacuc, UGAguaacag, AAGguacccg, AGGguaggcu, GGGgcaggac, CCUguaagug, AUUguaagug, ACUguacgag, GUAguagugu, AGAguaugag, UCAguguggg, UGGguauaua, UAGguagcua, GGGguaaaga, AGGguuacuu, CAUguaaaug, GGAguaguaa, CAGgucaauc, CGGguuagug, UAGguacaug, UAGguuaaga, UGGguaccuu, CGGguggaca, CAGgucuuac, AAGguggagc, AUGguaacca, UCGguaaguu, UAUguacaaa, AAUguagauu, GUAgcuagua, AAGguauugg, GAGgucuuug, GAAguucagg, UGGguaucac, AGAguacugg, CAGguuaaug, AGGguacgug, AGGgcacagg, CUGguuaguu, UUGguacgag, ACGgugauca, CCUgugagag, GAGgugaagu, AAGguacauc, UCUguaugug, UUGguggaag, UGGgcagguu, GAAguggagc, ACAguaagac, CGGguaccaa, CAAguacguc, AGAgugaggg, CGGguaagaa, AAUguaggug, AUCgugugcu, UAGgucaugg, CAGguuuuga, AAGgcaugca, GAGgugcugc, AAGguuaaua, CAGguucauc, GCGguaggug, GACgugagua, CAGgucuacu, UUGguaugag, AGCgugggca, AUGguaaggu, AUGguaccuc, UUGguauggu, UAUguaugaa, UGGguauggg, GAUguaaaua, CCGguaaguu, GAGgucugaa, GAGgugcgag, CUGgucagcc, CAGguuuugu, CGGguggugu, UAAguuagua, UUUgugugug, CAGguuaacc, UUGguacuuu, GCUguaaggc, AGGguggcug, GAUguaaaaa, AAGgucaaaa, CAGguagcgc, CAGguuuggc, GAGgugguuu, CGGguaaaua, CUGguucggu, GGAgugagcc, AAGgugcgcg, GAAguacauc, AGUgucugua, CCCgugagcu, GAGguucaca, CUAgugggua, GAGguaacua, UCGguauguc, UAAguauuug, CAGguaagcg, GAGgugguaa, CGAguaagag, CCGguaagcu, GAGgucuugu, AAGguggguc, CACguaagug, AGUguaauga, AAAgugugua, GGAgugccaa, CACgugaguu, AAGguuggau, UAUguaaaua, CUGguaggaa, UAUguaaacu, AAUguauuuu, CUGgcaagug, UGUgugguau, UAUguauguu, UUGgugacuc, GGAguaaggu, AAGguagaug, UGGguagggu, AAUguaauuc, GUGguauggc, GGAguggguu, AGGguaccac, UAGgugacag, ACAguaggca, AUGguuugaa, GCAguaacua, CCGguaggua, AGAguaggcc, AAGguugaca, CUGgugugua, GAAgucuguc, UGGgcucgga, CAGguagccu, AGAguaggua, UAAguauguc, CUGguauauc, GAGguguguu, AUGgugcaug, AAGguacgcc, UGAguaacua, GAGgugacag, GUUguccugu, UUGgugucuu, AAUgugaagg, UUGguggaua, UAGguguguu, CUGgcaaguu, GCAguaagau, GCGguggaaa, UGCguccagc, AAAguggagu, CGUgugagcc, AGAguacugu, CAGguauagc, UACguaagga, AAGgucuuua, AAGguggucu, GGGguaaauu, UCAgugagga, AGAguacguu, GAGgucguca, UAGguuugau, CAUguaaacc, AAGguggcac, CAGguagaug, AACguaaaag, UAGgucucug, AUAguaggug, UAGgcaagag, UAGgcacggc, AAGgucuuca, CCAguaugcu, CAAgugaguu, CAGgucucaa, CAGguuacau, GGAgugagca, AGAguacgca, CUGguguugg, AAGguacuca, CUAguaaggg, AGAguaaaag, AAGguaacga, CUGguccccg, UAAguauggg, GAGgucgagc, UUGguauaua, AAAgucaagg, AAGgucuagg, CGAguagguc, AGGguucguu, GAGgcaggcc, CUAguauuac, ACGguaugug, UAGgugguuc, AGAguauaac, UUGgugcguc, ACCguuaucu, CCAgugauga, GAAguaugca, GAAguauggc, CCGguaggac, AAUguaagca, AGAguaauug, AGGguugguu, GUGguaggag, AAGgcaguuu, CAAguaagcc, CUGgcaagua, CAGgcaugau, AGGguaauug, GGGguaaccu, AAAguaacua, UAGgucugcc, ACGguaugaa, AGUguauggg, UGGguuggca, UAGguaaacu, AGAgugggua, AGAguauuug, AGUguaggaa, CUUguacgua, GAUgugagau, CAGgcagcca, AAGgucacug, AAGgucugac, UAGguuccuu, CUGgugcuuu, UGAguuggug, UUGgugggau, UGAguagggu, UCGgugaggu, AAAguaaaga, AAGgcaaguc, CGGguaaagc, AAAguuaguu, UUAguaagca, GAGgucacau, UAAgugguau, UAGgugcuuu, GGAguaggca, UGAguaagga, CAGguggagc, GAUguagaag, AAUgccugcc, AUGguaaggc, UGGguaauau, CUGguaccuc, CACgugagcc, UGAguuugug, CCGguagugu, AAAgugacaa, GAAguggguu, CAGgugcagc, GAGgugggcc, UAUgugcguc, GGGguacugg, CUGguagguu, UUGgcauguu, AAUguaauac, UAGgccggug, AGAgucagua, UAAguaaauc, CAGguuccuc, UAGguacgau, AGAguuagug, GCAguaagug, AGGgugguag, GGAguaaugu, GAUguaaguc, CCAguuucgu, AAGguucggg, AUGguggagu, AAGguaccgg, GAAgugcgaa, UGGgucaguu, AAGguguaga, UGGguaggcc, CCAgugaguc, AAGgucacuu, AGCgugaggc, UCCgugguaa, AGAguacuua, GGGgucagau, AAGguggacc, AGAgugagcg, AGAgucagau, UAAguauuac, AGAguauuuc, AGAguucagc, AUGgugaagu, UAGgugaucc, GGAguaagau, UAGguaccaa, AGAguugguc, GAAgugagac, AUCguagguu, GAGguacgcu, ACGguaaggg, CAGgcauguc, UUAguaagau, UGAguagguu, AGGguacgaa, ACGguauguu, AGGguacugu, UUGguaugga, UAAguaacug, GCGgucagcc, UUUgugaguc, GUGgucagug, CUGgucugua, GAGguucuua, AUGguacuga, AAUgugcuuu, AGGguggcgu, CCGgcaggaa, CAUguggguc, UUGguuuguu, CAGguucugu, ACGguaagcg, CUGgucagua, UCAguaggcu, UGAguaggac, CAGguuuuaa, GAGguguccc, AGGguggguu, GUGgugagac, CACguaggga, GUGguauuuu, GAGauauccu, AAGgugaaca, UAAguagggc, CUGgugcggg, CUGgucaaua, AGAguaaaaa, AAGgugcagu, CGGguaagca, AAAgugagcc, AUGguaauca, GCAguacgug, AUGguacaug, AAGguuaaga, CGGguaaaug, GAGguucgca, GAGgcucugg, AUGgugggac, AACgugguag, AAGgugauag, GGGguuugca, CAUguaaggg, UCAguugagu, AAAgugcggc, AGAgugagcc, AUGgcaagaa, ACAguaaggu, AAGgucucua, GUGguaaaaa, AAAguaggug, UAGgugcacu, GUCgugguau, CAGguauagg, UGAgugagag, ACUgugagcc, AUCguuaguu, UUUguaccaa, UGGgugagau, AGAgugagaa, AGAguagggg, AGGgcaagua, CGGgucagua, UUGguaugcc, CGGguuagau, GGGgugaagu, CCCgugugaa, GCAguuugga, UGCguaagac, AGAgucugua, CACgugagca, AGGguaaaag, CAGgcugggu, GAAgucuuca, AAGgcaaaaa, GUAguaaaua, CUAgugagag, GAAguuucug, CCUguacgua, GAGgugcgcg, AAGguguaaa, CCAguauguu, CCGgucagcu, AUGguuccug, CAAguuaaau, AGAguaggcu, AUGgugggca, GGAguaagac, AGGgucacga, UAGgugauau, GAAguaaguc, CGGguaagau, CAAguagcua, UGAguaaaau, GUCguacgug, AUGguacgua, CAGgucucgg, GAGgcauguc, AGAgugggau, GUGguuagag, UGGgugguga, AAGguuaaac, CUUguuagcu, AAAguaggaa, UAGguuguau, AGGgugcgcc, AAGgugggcu, UAAguaucug, AAGguaacgu, AUGguggggc, CAAguacacg, GGCguaagug, AUAguaggac, AGAgugaggu, UUUguaaaaa, GAAguuugua, CUAguaaucu, AAGguuuuua, GAGgugcguu, UAGgcgagua, ACCgugagua, CAGgucccga, AUGguacugg, UGAguucagu, AAUguguggu, UCCguugguu, CAGgucagag, CAGgucccua, UAGguagacu, CAAguuaagg, GAGgugugcg, GAAgcugccc, CGAguacgug, CGGguaggua, UUGguauuga, AUUguaugau, UUGguaugaa, GAGgugguca, GCUguaugaa, CAGguguugc, CAGguaaaac, AUAguaaggu, CUGguuagag, AGCgugugag, AAGguuaucu, CACgugagua, AGGgucagua, GAGguauaau, CAGguuauuu, AGGguggacu, AUUguaauuc, UUUguggguu, AUGguacgug, AAGguguucc, CAGgugacgc, GAGguacuaa, ACAguucagu, GAGgucacgg, CAAguaaggc, AAGguuuggg, AAAgugggcu, GCGguucuug, GAGguggagc, UGAgucagug, CAGgucaagg, AGUguaagcu, GAGgcagaaa, AAGgucacac, GAAguagguu, GUCguaaguu, AGAguaugca, CCUgugcaaa, ACGgugaaaa, CAGguacgaa, CAUgugagga, AGCgugagua, GGUguguagg, AACgugagcu, GAGgugaacu, AGAguucagu, AACgugugua, CAGguugugg, AAGguacuag, UCAgugaaaa, AAUgucuggu, ACGguaaaau, CUGguguaag, GAGgugcgaa, AGGguuucuc, CAGguagccc, AUUguauugg, AUGguacuua, GAGgcccgac, UCGguaagac, CGGgcuguag, UAUgugugug, UAGguagaaa, GUGgucauua, UAGgugaaag, ACUguaauuc, GCAguacagg, UCGgugaguc, UAUguaggga, AUGguauguc, GUGgugugug, CUGgugaccu, AAUgugaaua, UAGgucucac, GAGguuauug, UGAguaggcu, CGGgcacgua, GCAguaaaua, CCGgugagag, UAAguugguc, CCGgugagcc, AAGguuguca, CUGguauuau, GGGguauggg, AAAgucagua, UUUguaugua, UAAguacugc, CAGguaccaa, GAAguucaga, AUGgugcggu, GUGgugaggu, UGAguaagcc, UAUguaaggg, GUGguggaaa, GAGgugauug, GGAguuugua, AAGgucacga, GUGguagagg, UAAguauauc, AAGgugucca, UAUgugguau, GAGguacaau, AAGguggggg, GGAguaggug, and UAGgugacuu.

In some embodiments, the splice site sequence (e.g., 5′ splice site sequence) comprises AGA. In some embodiments, the splice site sequence (e.g., 5′ splice site sequence) comprises AAA. In some embodiments, the splice site sequence (e.g., 5′ splice site sequence) comprises AAC. In some embodiments, the splice site sequence (e.g., 5′ splice site sequence) comprises AAU. In some embodiments, the splice site sequence (e.g., 5′ splice site sequence) comprises AAG. In some embodiments, the splice site sequence (e.g., 5′ splice site sequence) comprises ACA. In some embodiments, the splice site sequence (e.g., 5′ splice site sequence) comprises AUA. In some embodiments, the splice site sequence (e.g., 5′ splice site sequence) comprises AUU. In some embodiments, the splice site sequence (e.g., 5′ splice site sequence) comprises AUG. In some embodiments, the splice site sequence (e.g., 5′ splice site sequence) comprises AUC. In some embodiments, the splice site sequence (e.g., 5′ splice site sequence) comprises CAA. In some embodiments, the splice site sequence (e.g., 5′ splice site sequence) comprises CAU. In some embodiments, the splice site sequence (e.g., 5′ splice site sequence) comprises CAC. In some embodiments, the splice site sequence (e.g., 5′ splice site sequence) comprises CAG. In some embodiments, the splice site sequence (e.g., 5′ splice site sequence) comprises GAA. In some embodiments, the splice site sequence (e.g., 5′ splice site sequence) comprises GAC. In some embodiments, the splice site sequence (e.g., 5′ splice site sequence) comprises GAU. In some embodiments, the splice site sequence (e.g., 5′ splice site sequence) comprises GAG. In some embodiments, the splice site sequence (e.g., 5′ splice site sequence) comprises GGA. In some embodiments, the splice site sequence (e.g., 5′ splice site sequence) comprises GCA. In some embodiments, the splice site sequence (e.g., 5′ splice site sequence) comprises GGG. In some embodiments, the splice site sequence (e.g., 5′ splice site sequence) comprises GGC. In some embodiments, the splice site sequence (e.g., 5′ splice site sequence) comprises GUU. In some embodiments, the splice site sequence (e.g., 5′ splice site sequence) comprises GGU. In some embodiments, the splice site sequence (e.g., 5′ splice site sequence) comprises GUC. In some embodiments, the splice site sequence (e.g., 5′ splice site sequence) comprises GUA. In some embodiments, the splice site sequence (e.g., 5′ splice site sequence) comprises GUG. In some embodiments, the splice site sequence (e.g., 5′ splice site sequence) comprises UCU. In some embodiments, the splice site sequence (e.g., 5′ splice site sequence) comprises UCC. In some embodiments, the splice site sequence (e.g., 5′ splice site sequence) comprises UCA. In some embodiments, the splice site sequence (e.g., 5′ splice site sequence) comprises UCG. In some embodiments, the splice site sequence (e.g., 5′ splice site sequence) comprises UUU. In some embodiments, the splice site sequence (e.g., 5′ splice site sequence) comprises UUC. In some embodiments, the splice site sequence (e.g., 5′ splice site sequence) comprises UUA. In some embodiments, the splice site sequence (e.g., 5′ splice site sequence) comprises UUG. In some embodiments, the splice site sequence (e.g., 5′ splice site sequence) comprises UGU. In some embodiments, the splice site sequence (e.g., 5′ splice site sequence) comprises UAU. In some embodiments, the splice site sequence (e.g., 5′ splice site sequence) comprises GGA. In some embodiments, the splice site sequence (e.g., 5′ splice site sequence) comprises CUU. In some embodiments, the splice site sequence (e.g., 5′ splice site sequence) comprises CUC. In some embodiments, the splice site sequence (e.g., 5′ splice site sequence) comprises CUA. In some embodiments, the splice site sequence (e.g., 5′ splice site sequence) comprises CUG. In some embodiments, the splice site sequence (e.g., 5′ splice site sequence) comprises CCU. In some embodiments, the splice site sequence (e.g., 5′ splice site sequence) comprises CCC. In some embodiments, the splice site sequence (e.g., 5′ splice site sequence) comprises CCA. In some embodiments, the splice site sequence (e.g., 5′ splice site sequence) comprises CCG. In some embodiments, the splice site sequence (e.g., 5′ splice site sequence) comprises ACU. In some embodiments, the splice site sequence (e.g., 5′ splice site sequence) comprises ACC. In some embodiments, the splice site sequence (e.g., 5′ splice site sequence) comprises ACG. In some embodiments, the splice site sequence (e.g., 5′ splice site sequence) comprises AGC. In some embodiments, the splice site sequence (e.g., 5′ splice site sequence) comprises AGU. In some embodiments, the splice site sequence (e.g., 5′ splice site sequence) comprises AGG. In some embodiments, the splice site sequence (e.g., 5′ splice site sequence) comprises CGU. In some embodiments, the splice site sequence (e.g., 5′ splice site sequence) comprises UAC. In some embodiments, the splice site sequence (e.g., 5′ splice site sequence) comprises UAA. In some embodiments, the splice site sequence (e.g., 5′ splice site sequence) comprises UAG. In some embodiments, the splice site sequence (e.g., 5′ splice site sequence) comprises CGC. In some embodiments, the splice site sequence (e.g., 5′ splice site sequence) comprises CGA. In some embodiments, the splice site sequence (e.g., 5′ splice site sequence) comprises CGG. In some embodiments, the splice site sequence comprises AGAguaaggg.

In an embodiment, a gene sequence or splice site sequence provided herein is related to a proliferative disease, disorder, or condition (e.g., cancer, benign neoplasm, or inflammatory disease). In an embodiment, a gene sequence or splice site sequence provided herein is related to a non-proliferative disease, disorder, or condition. In an embodiment, a gene sequence or splice site sequence provided herein is related to a neurological disease or disorder; autoimmune disease or disorder; immunodeficiency disease or disorder; lysosomal storage disease or disorder; cardiovascular condition, disease or disorder; metabolic disease or disorder; respiratory condition, disease, or disorder; renal disease or disorder; or infectious disease in a subject. In an embodiment, a gene sequence or splice site sequence provided herein is related to a neurological disease or disorder (e.g., Huntington's disease). In an embodiment, a gene sequence or splice site sequence provided herein is related to an immunodeficiency disease or disorder. In an embodiment, a gene sequence or splice site sequence provided herein is related to a lysosomal storage disease or disorder. In an embodiment, a gene sequence or splice site sequence provided herein is related to a cardiovascular condition, disease or disorder. In an embodiment, a gene sequence or splice site sequence provided herein is related to a metabolic disease or disorder. In an embodiment, a gene sequence or splice site sequence provided herein is related to a respiratory condition, disease, or disorder. In an embodiment, a gene sequence or splice site sequence provided herein is related to a renal disease or disorder. In an embodiment, a gene sequence or splice site sequence provided herein is related to an infectious disease.

In an embodiment, a gene sequence or splice site sequence provided herein is related to a mental retardation disorder. In an embodiment, a gene sequence or splice site sequence provided herein is related to a mutation in the SETD5 gene. In an embodiment, a gene sequence or splice site sequence provided herein is related to an immunodeficiency disorder. In an embodiment, a gene sequence and splice site sequence provided herein is related to a mutation in the GATA2 gene.

In some embodiments, a compound of Formula (I) described herein interacts with (e.g., binds to) a splicing complex component (e.g., a nucleic acid (e.g., an RNA) or a protein). In some embodiments, the splicing complex component is selected from 9G8, A1 hnRNP, A2 hnRNP, ASD-1, ASD-2b, ASF, BRR2, B1 hnRNP, C1 hnRNP, C2 hnRNP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, LUC7L, M hnRNP, mBBP, muscle-blind like (MBNL), NF45, NFAR, Nova-1, Nova-2, nPTB, P54/SFRS11, polypyrimidine tract binding protein (PTB), a PRP protein (e.g., PRP8, PRP6, PRP31, PRP4, PRP3, PRP28, PRP5, PRP2, PRP19), PRP19 complex proteins, RBM42, R hnRNP, RNPC1, SAD1, SAM68, SC35, SF, SF1/BBP, SF2, SF3A complex, SF3B complex, SFRS10, an Sm protein (such as B, D1, D2, D3, F, E, G), SNU17, SNU66, SNU114, an SR protein, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, U1 snRNP, U11 snRNP, U12 snRNP, U1-70K, U1-A, U1-C, U2 snRNP, U2AF1-RS2, U2AF35, U2AF65, U4 snRNP, U5 snRNP, U6 snRNP, Urp, and YB1.

In some embodiments, the splicing complex component comprises RNA (e.g., snRNA). In some embodiments, a compound described herein binds to a splicing complex component comprising snRNA. The snRNA may be selected from, e.g., U1 snRNA, U2 snRNA, U4 snRNA, U5 snRNA, U6 snRNA, U11 snRNA, U12 snRNA, U4atac snRNA, and any combination thereof.

In some embodiments, the splicing complex component comprises a protein, e.g., a protein associated with an snRNA. In some embodiments, the protein comprises SC35, SRp55, SRp40, SRm300, SFRS10, TASR-1, TASR-2, SF2/ASF, 9G8, SRp75, SRp30c, SRp20 and P54/SFRS11. In some embodiments, the splicing complex component comprises a U2 snRNA auxiliary factor (e.g., U2AF65, U2AF35), Urp/U2AF1-RS2, SF1/BBP, CBP80, CBP 20, SF1 or PTB/hnRNP1. In some embodiments, the splicing complex component comprises a heterogenous ribonucleoprotein particle (hnRNP), e.g., an hnRNP protein. In some embodiments, the hnRNP protein comprises A1, A2/B1, L, M, K, U, F, H, G, R, I or C1/C2. Human genes encoding hnRNPs include HNRNPA0, HNRNPA1, HNRNPA1L1, HNRNPA1L2, HNRNPA3, HNRNPA2B1, HNRNPAB, HNRNPB1, HNRNPC, HNRNPCL1, HNRNPD, HNRPDL, HNRNPF, HNRNPH1, HNRNPH2, HNRNPH3, HNRNPK, HNRNPL, HNRPLL, HNRNPM, HNRNPR, HNRNPU, HNRNPUL1, HNRNPUL2, HNRNPUL3, and FMRL.

In one aspect, the compounds of Formula (I) and pharmaceutically acceptable salts, solvates, hydrates, tautomers, stereoisomers, and compositions thereof, may modulate (e.g., increase or decrease) a splicing event of a target nucleic acid sequence (e.g., DNA, RNA, or a pre-mRNA), for example, a nucleic acid encoding a gene described herein, or a nucleic acid encoding a protein described herein, or a nucleic acid comprising a splice site described herein. In an embodiment, the splicing event is an alternative splicing event.

In an embodiment, the compound of Formula (I) or a pharmaceutically acceptable salt, solvate, hydrate, tautomer, stereoisomer, and compositions thereof increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR. In an embodiment, the compound of Formula (I) or a pharmaceutically acceptable salt, solvate, hydrate, tautomer, stereoisomer, and compositions thereof decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR.

In another aspect, the present disclosure features a method of forming a complex comprising a component of a spliceosome (e.g., a major spliceosome component or a minor spliceosome component), a nucleic acid (e.g., a DNA, RNA, e.g., a pre-mRNA), and a compound of Formula (I) or a pharmaceutically acceptable salt, solvate, hydrate, tautomer, stereoisomer, or composition thereof, comprising contacting the nucleic acid (e.g., a DNA, RNA, e.g., a pre-mRNA) with said compound of Formula (I). In an embodiment, the component of a spliceosome is selected from the U1, U2, U4, U5, U6, U11, U12, U4atac, U6atac small nuclear ribonucleoproteins (snRNPs), or a related accessory factor. In an embodiment, the component of a spliceosome is recruited to the nucleic acid in the presence of the compound of Formula (I), or a pharmaceutically acceptable salt, solvate, hydrate, tautomer, stereoisomer, or composition thereof.

In another aspect, the present disclosure features a method of altering the structure or conformation of a nucleic acid (e.g., a DNA, RNA, e.g., a pre-mRNA) comprising contacting the nucleic acid with a compound of Formula (I) or a pharmaceutically acceptable salt, solvate, hydrate, tautomer, stereoisomer, or composition thereof. In an embodiment, the altering comprises forming a bulge or kink in the nucleic acid (e.g., a DNA, RNA, e.g., a pre-mRNA). In an embodiment, the altering comprises stabilizing a bulge or a kink in the nucleic acid (e.g., a DNA, RNA, e.g., a pre-mRNA). In an embodiment, the altering comprises reducing a bulge or a kink in the nucleic acid (e.g., a DNA, RNA, e.g., a pre-mRNA). In an embodiment, the nucleic acid (e.g., a DNA, RNA, e.g., a pre-mRNA) comprises a splice site. In an embodiment, the compound of Formula (I) interacts with a nucleobase, ribose, or phosphate moiety of a nucleic acid (e.g., a DNA, RNA, e.g., pre-mRNA).

The present disclosure also provides methods for the treatment or prevention of a disease, disorder, or condition. In an embodiment, the disease, disorder or condition is related to (e.g., caused by) a splicing event, such as an unwanted, aberrant, or alternative splicing event. In an embodiment, the disease, disorder or condition comprises a proliferative disease (e.g., cancer, benign neoplasm, or inflammatory disease) or non-proliferative disease. In an embodiment, the disease, disorder, or condition comprises a neurological disease, autoimmune disorder, immunodeficiency disorder, cardiovascular condition, metabolic disorder, lysosomal storage disease, respiratory condition, renal disease, or infectious disease in a subject. In another embodiment, the disease, disorder, or condition comprises a haploinsufficiency disease, an autosomal recessive disease (e.g., with residual function), or a paralogue activation disorder. In another embodiment, the disease, disorder, or condition comprises an autosomal dominant disorder (e.g., with residual function). Such methods comprise the step of administering to the subject in need thereof an effective amount of a compound of Formula (I), or a pharmaceutically acceptable salt, solvate, hydrate, tautomer, stereoisomer thereof, or a pharmaceutical composition thereof. In certain embodiments, the methods described herein include administering to a subject an effective amount of a compound of Formula (I), or a pharmaceutically acceptable salt thereof, or a pharmaceutical composition thereof.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

A proliferative disease, disorder, or condition may also be associated with inhibition of apoptosis of a cell in a biological sample or subject. All types of biological samples described herein or known in the art are contemplated as being within the scope of the disclosure. The compounds of Formula (I) and pharmaceutically acceptable salts, solvates, hydrates, tautomers, stereoisomers, and compositions thereof, may induce apoptosis, and therefore, be useful in treating and/or preventing proliferative diseases, disorders, or conditions.

In certain embodiments, the proliferative disease to be treated or prevented using the compounds of Formula (I) is cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In some embodiments, the proliferative disease is associated with angiogenesis. All types of angiogenesis disclosed herein or known in the art are contemplated as being within the scope of the disclosure.

In some embodiments, the compound of Formula (I), or a pharmaceutically acceptable salt thereof, or compositions comprising such compound or pharmaceutically acceptable salt thereof, is used to prevent or treat a non-proliferative disease. Exemplary non-proliferative diseases include a neurological disease, autoimmune disorder, immunodeficiency disorder, lysosomal storage disease, cardiovascular condition, metabolic disorder, respiratory condition, inflammatory disease, renal disease, or infectious disease.

In certain embodiments, the non-proliferative disease is a neurological disease. In certain embodiments, the compound of Formula (I), or a pharmaceutically acceptable salt thereof, or compositions comprising such compound or pharmaceutically acceptable salt thereof, is used to prevent or treat a neurological disease, disorder, or condition. A neurological disease, disorder, or condition may include a neurodegenerative disease, a psychiatric condition, or a musculoskeletal disease. A neurological disease may further include a repeat expansion disease, e.g., which may be characterized by the expansion of a nucleic acid sequence in the genome. For example, a repeat expansion disease includes myotonic dystrophy, amyotrophic lateral sclerosis, Huntington's disease, a trinucleotide repeat disease, or a polyglutamine disorder (e.g., ataxia, fragile X syndrome). In some embodiments, the neurological disease comprises a repeat expansion disease, e.g., Huntington's disease. Additional neurological diseases, disorders, and conditions include Alzheimer's disease, Huntington's chorea, a prion disease (e.g., Creutzfeld-Jacob disease, bovine spongiform encephalopathy, Kuru, or scrapie), a mental retardation disorder (e.g., a disorder caused by a SETD5 gene mutation, e.g., intellectual disability-facial dysmorphism syndrome, autism spectrum disorder), Lewy Body disease, diffuse Lewy body disease (DLBD), dementia, progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, Parkinson's disease, Down's syndrome, multiple system atrophy, spinal muscular atrophy (SMA), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, a demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, Machado-Joseph disease, or a combination thereof. In some embodiments, the neurological disease comprises Friedrich's ataxia or Sturge Weber syndrome. In some embodiments, the neurological disease comprises Huntington's disease. All types of neurological diseases disclosed herein or known in the art are contemplated as being within the scope of the disclosure.

In certain embodiments, the non-proliferative disease is an autoimmune disorder or an immunodeficiency disorder. In certain embodiments, the compound of Formula (I), or a pharmaceutically acceptable salt thereof, or compositions comprising such compound or pharmaceutically acceptable salt thereof, is used to prevent or treat an autoimmune disease, disorder, or condition, or an immunodeficiency disease, disorder, or condition. Exemplary autoimmune and immunodeficiency diseases, disorders, and conditions include arthritis (e.g., rheumatoid arthritis, osteoarthritis, gout), Chagas disease, chronic obstructive pulmonary disease (COPD), dermatomyositis, diabetes mellitus type 1, endometriosis, Goodpasture's syndrome, Graves' disease, Guillain-Barre syndrome (GBS), Hashiomoto's disease, Hidradenitis suppurativa, Kawasaki disease, ankylosing spondylitis, IgA nephropathy, idiopathic thrombocytopenic purpura, inflammatory bowel disease, Crohn's disease, ulcerative colitis, collagenous colitis, lymphocytic colitis, ischemic colitis, diversion colitis, Behcet's syndrome, infective colitis, indeterminate colitisinterstitial cystitis, lupus (e.g., systemic lupus erythematosus, discoid lupus, drug-induced lupus, neonatal lupus), mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, vasculitis, vitiligo, a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), neutropenia, aplastic anemia, and Wegener's granulomatosis. In some embodiments, the autoimmune or immunodeficiency disorder comprises chronic mucocutaneous candidiasis. All types of autoimmune disorders and immunodeficiency disorders disclosed herein or known in the art are contemplated as being within the scope of the disclosure.

In certain embodiments, the non-proliferative disease is a cardiovascular condition. In certain embodiments, the compound of Formula (I), or a pharmaceutically acceptable salt thereof, or compositions comprising such compound or pharmaceutically acceptable salt thereof, is used to prevent or treat a cardiovascular disease, disorder, or condition. A cardiovascular disease, disorder, or condition may include a condition relating to the heart or vascular system, such as the arteries, veins, or blood. Exemplary cardiovascular diseases, disorders, or conditions include angina, arrhythmias (atrial or ventricular or both), heart failure, arteriosclerosis, atheroma, atherosclerosis, cardiac hypertrophy, cardiac or vascular aneurysm, cardiac myocyte dysfunction, carotid obstructive disease, endothelial damage after PTCA (percutaneous transluminal coronary angioplasty), hypertension including essential hypertension, pulmonary hypertension and secondary hypertension (renovascular hypertension, chronic glomerulonephritis), myocardial infarction, myocardial ischemia, peripheral obstructive arteriopathy of a limb, an organ, or a tissue; peripheral artery occlusive disease (PAOD), reperfusion injury following ischemia of the brain, heart or other organ or tissue, restenosis, stroke, thrombosis, transient ischemic attack (TIA), vascular occlusion, vasculitis, and vasoconstriction. All types of cardiovascular diseases, disorders, or conditions disclosed herein or known in the art are contemplated as being within the scope of the disclosure.

In certain embodiments, the non-proliferative disease is a metabolic disorder. In certain embodiments, the compound of Formula (I), or a pharmaceutically acceptable salt thereof, or compositions comprising such compound or pharmaceutically acceptable salt thereof, is used to prevent or treat a metabolic disease, disorder, or condition. A metabolic disease, disorder, or condition may include a disorder or condition that is characterized by abnormal metabolism, such as those disorders relating to the consumption of food and water, digestion, nutrient processing, and waste removal. A metabolic disease, disorder, or condition may include an acid-base imbalance, a mitochondrial disease, a wasting syndrome, a malabsorption disorder, an iron metabolism disorder, a calcium metabolism disorder, a DNA repair deficiency disorder, a glucose metabolism disorder, hyperlactatemia, a disorder of the gut microbiota. Exemplary metabolic conditions include obesity, diabetes (Type I or Type II), insulin resistance, glucose intolerance, lactose intolerance, eczema, hypertension, Hunter syndrome, Krabbe disease, sickle cell anemia, maple syrup urine disease, Pompe disease, and metachromatic leukodystrophy. All types of metabolic diseases, disorders, or conditions disclosed herein or known in the art are contemplated as being within the scope of the disclosure.

In certain embodiments, the non-proliferative disease is a respiratory condition. In certain embodiments, the compound of Formula (I), or a pharmaceutically acceptable salt thereof, or compositions comprising such compound or pharmaceutically acceptable salt thereof, is used to prevent or treat a respiratory disease, disorder, or condition. A respiratory disease, disorder, or condition can include a disorder or condition relating to any part of the respiratory system, such as the lungs, alveoli, trachea, bronchi, nasal passages, or nose. Exemplary respiratory diseases, disorders, or conditions include asthma, allergies, bronchitis, allergic rhinitis, chronic obstructive pulmonary disease (COPD), lung cancer, oxygen toxicity, emphysema, chronic bronchitis, and acute respiratory distress syndrome. All types of respiratory diseases, disorders, or conditions disclosed herein or known in the art are contemplated as being within the scope of the disclosure.

In certain embodiments, the non-proliferative disease is a renal disease. In certain embodiments, the compound of Formula (I), or a pharmaceutically acceptable salt thereof, or compositions comprising such compound or pharmaceutically acceptable salt thereof, is used to prevent or treat a renal disease, disorder, or condition. A renal disease, disorder, or condition can include a disease, disorder, or condition relating to any part of the waste production, storage, and removal system, including the kidneys, ureter, bladder, urethra, adrenal gland, and pelvis. Exemplary renal diseases include acute kidney failure, amyloidosis, Alport syndrome, adenovirus nephritis, acute lobar nephronia, tubular necrosis, glomerulonephritis, kidney stones, urinary tract infections, chronic kidney disease, polycystic kidney disease, and focal segmental glomerulosclerosis (FSGS). In some embodiments, the renal disease, disorder, or condition comprises HIV-associated nephropathy or hypertensive nephropathy. All types of renal diseases, disorders, or conditions disclosed herein or known in the art are contemplated as being within the scope of the disclosure.

In certain embodiments, the non-proliferative disease is an infectious disease. In certain embodiments, the compound of Formula (I), or a pharmaceutically acceptable salt thereof, or compositions comprising such compound or pharmaceutically acceptable salt thereof, is used to prevent or treat an infectious disease, disorder, or condition. An infectious disease may be caused by a pathogen such as a virus or bacteria. Exemplary infectious diseases include human immunodeficiency syndrome (HIV), acquired immunodeficiency syndrome (AIDS), meningitis, African sleeping sickness, actinomycosis, pneumonia, botulism, chlamydia, Chagas disease, Colorado tick fever, cholera, typhus, giardiasis, food poisoning, ebola hemorrhagic fever, diphtheria, Dengue fever, gonorrhea, streptococcal infection (e.g., Group A or Group B), hepatitis A, hepatitis B, hepatitis C, herpes simplex, hookworm infection, influenza, Epstein-Barr infection, Kawasaki disease, kuru, leprosy, leishmaniasis, measles, mumps, norovirus, meningococcal disease, malaria, Lyme disease, listeriosis, rabies, rhinovirus, rubella, tetanus, shingles, scarlet fever, scabies, Zika fever, yellow fever, tuberculosis, toxoplasmosis, or tularemia. In some embodiments, the infectious disease comprises cytomegalovirus. All types of infectious diseases, disorders, or conditions disclosed herein or known in the art are contemplated as being within the scope of the disclosure.

In certain embodiments, the disease, disorder, or condition is a haploinsufficiency disease. In certain embodiments, the compound of Formula (I), or a pharmaceutically acceptable salt thereof, or compositions comprising such compound or pharmaceutically acceptable salt thereof, is used to prevent or treat a haploinsufficiency disease, disorder, or condition. A haploinsufficiency disease, disorder, or condition may refer to a monogenic disease in which an allele of a gene has a loss-of-function lesion, e.g., a total loss of function lesion. In an embodiment, the loss-of-function lesion is present in an autosomal dominant inheritance pattern or is derived from a sporadic event. In an embodiment, the reduction of gene product function due to the altered allele drives the disease phenotype despite the remaining functional allele (i.e. said disease is haploinsufficient with regard to the gene in question). In an embodiment, a compound of Formula (I) increases expression of the haploinsufficient gene locus. In an embodiment, a compound of Formula (I) increases one or both alleles at the haploinsufficient gene locus. Exemplary haploinsufficiency diseases, disorders, and conditions include Robinow syndrome, cardiomyopathy, cerebellar ataxia, pheochromocytoma, Charcot-Marie-Tooth disease, neuropathy, Takenouchi-Kosaki syndrome, Coffin-Siris syndrome 2, chromosome 1p35 deletion syndrome, spinocerebellar ataxia 47, deafness, seizures, dystonia 9, GLUT1 deficiency syndrome 1, GLUT1 deficiency syndrome 2, stomatin-deficient cryohydrocytosis, basal cell carcinoma, basal cell nevus syndrome, medulloblastoma, somatic, brain malformations, macular degeneration, cone-rod dystrophy, Dejerine-Sottas disease, hypomyelinating neuropathy, Roussy-Levy syndrome, glaucoma, autoimmune lymphoproliferative syndrome, pituitary hormone deficiency, epileptic encephalopathy, early infantile, popliteal pterygium syndrome, van der Woude syndrome, Loeys-Dietz syndrome, Skraban-Deardorff syndrome, erythrocytosis, megalencephaly-polymicrogyria-polydactyly-hydrocephalus syndrome, mental retardation, CINCA syndrome, familial cold inflammatory syndrome 1, keratoendothelitis fugax hereditaria, Muckle-Wells syndrome, Feingold syndrome 1, Acute myeloid leukemia, Heyn-Sproul-Jackson syndrome, Tatton-Brown-Rahman syndrome, Shashi-Pena syndrome, Spastic paraplegia, autosomal dominant, macrophthalmia, colobomatous, with microcornea, holoprosencephaly, schizencephaly, endometrial cancer, familial, colorectal cancer, hereditary nonpolyposis, intellectual developmental disorder with dysmorphic facies and behavioral abnormalities, ovarian hyperstimulation syndrome, schizophrenia, Dias-Logan syndrome, premature ovarian failure, dystonia, dopa-responsive, due to sepiapterin reductase deficiency, Beck-Fahrner syndrome, chromosome 2p12-p11.2 deletion syndrome, neuronopathy, spastic paraplegia, familial adult myoclonic, colorectal cancer, hypothyroidism, Culler-Jones syndrome, holoprosencephaly, myelokathexis, WHIM syndrome, Mowat-Wilson syndrome, mental retardation, an intellectual developmental disorder, autism spectrum disorder, epilepsy, epileptic encephalopathy, Dravet syndrome, migraines, a mental retardation disorder (e.g., a disorder caused by a SETD5 gene mutation, e.g., intellectual disability-facial dysmorphism syndrome, autism spectrum disorder), a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), and febrile seizures.

In certain embodiments, the disease, disorder, or condition is an autosomal recessive disease, e.g., with residual function. In certain embodiments, the compound of Formula (I), or a pharmaceutically acceptable salt thereof, or compositions comprising such compound or pharmaceutically acceptable salt thereof, is used to prevent or treat an autosomal recessive disease, disorder, or condition. An autosomal recessive disease with residual function may refer to a monogenic disease with either homozygous recessive or compound heterozygous heritability. These diseases may also be characterized by insufficient gene product activity (e.g., a level of gene product greater than 0%). In an embodiment, a compound of Formula (I) may increase the expression of a target (e.g., a gene) related to an autosomal recessive disease with residual function. Exemplary autosomal recessive diseases with residual function include Friedreich's ataxia, Stargardt disease, Usher syndrome, chlorioderma, fragile X syndrome, achromatopsia 3, Hurler syndrome, hemophilia B, alpha-1-antitrypsin deficiency, Gaucher disease, X-linked retinoschisis, Wiskott-Aldrich syndrome, mucopolysaccharidosis (Sanfilippo B), DDC deficiency, epidermolysis bullosa dystrophica, Fabry disease, metachromatic leukodystrophy, and odontochondrodysplasia.

In certain embodiments, the disease, disorder, or condition is an autosomal dominant disease. In certain embodiments, the compound of Formula (I), or a pharmaceutically acceptable salt thereof, or compositions comprising such compound or pharmaceutically acceptable salt thereof, is used to prevent or treat an autosomal dominant disease, disorder, or condition. An autosomal dominant disease may refer to a monogenic disease in which the mutated gene is a dominant gene. These diseases may also be characterized by insufficient gene product activity (e.g., a level of gene product greater than 0%). In an embodiment, a compound of Formula (I) may increase the expression of a target (e.g., a gene) related to an autosomal dominant disease. Exemplary autosomal dominant diseases include Huntington's disease, achondroplasia, antithrombin III deficiency, Gilbert's disease, Ehlers-Danlos syndrome, hereditary hemorrhagic telangiectasia, intestinal polyposis, hereditary elliptosis, hereditary spherocytosis, marble bone disease, Marfan's syndrome, protein C deficiency, Treacher Collins syndrome, Von Willebrand's disease, tuberous sclerosis, osteogenesis imperfecta, polycystic kidney disease, neurofibromatosis, and idiopathic hypoparathyroidism.

In certain embodiments, the disease, disorder, or condition is a paralogue activation disorder. In certain embodiments, the compound of Formula (I), or a pharmaceutically acceptable salt thereof, or compositions comprising such compound or pharmaceutically acceptable salt thereof, is used to prevent or treat a paralogue activation disease, disorder, or condition. A paralogue activation disorder may comprise a homozygous mutation of genetic locus leading to loss-of-function for the gene product. In these disorders, there may exist a separate genetic locus encoding a protein with overlapping function (e.g. developmental paralogue), which is otherwise not expressed sufficiently to compensate for the mutated gene. In an embodiment, a compound of Formula (I) activates a gene connected with a paralogue activation disorder (e.g., a paralogue gene).

The cell described herein may be an abnormal cell. The cell may be in vitro or in vivo. In certain embodiments, the cell is a proliferative cell. In certain embodiments, the cell is a cancer cell. In certain embodiments, the cell is a non-proliferative cell. In certain embodiments, the cell is a blood cell. In certain embodiments, the cell is a lymphocyte. In certain embodiments, the cell is a benign neoplastic cell. In certain embodiments, the cell is an endothelial cell. In certain embodiments, the cell is an immune cell. In certain embodiments, the cell is a neuronal cell. In certain embodiments, the cell is a glial cell. In certain embodiments, the cell is a brain cell. In certain embodiments, the cell is a fibroblast. In certain embodiment, the cell is a primary cell, e.g., a cell isolated from a subject (e.g., a human subject).

In certain embodiments, the methods described herein comprise the additional step of administering one or more additional pharmaceutical agents in combination with the compound of Formula (I), a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment the modulation of splicing induced by the inventive compounds or compositions of this disclosure in the biological sample or subject. Thus, the combination of the inventive compounds or compositions and the additional pharmaceutical agent(s) may be useful in treating, for example, a cancer or other disease, disorder, or condition resistant to a treatment using the additional pharmaceutical agent(s) without the inventive compounds or compositions.

### Examples

In order that the invention described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC).

Proton NMR: ¹H NMR spectra were recorded in CDCl₃ solution in 5-mm o.d. tubes (Wildmad) at 24° C. and were collected on a BRUKER AVANCE NEO 400 at 400 MHz for ¹H. The chemical shifts (6) are reported relative to tetramethylsilane (TMS=0.00 ppm) and expressed in ppm.

LC/MS: Liquid chromatography-mass spectrometry (LC/MS) was performed on Shimadzu-2020EV using column: Shim-pack XR-ODS (C18, Ø4.6×50 mm, 3 m, 120 Å, 40° C.) operating in ESI(+) ionization mode; flow rate=1.2 mL/min. Mobile phase=0.05% TFA in water or CH₃CN; or on Shimadzu-2020EV using column: Poroshell HPH-C18 (C18, Ø4.6×50 mm, 3 m, 120 Å, 40° C.) operating in ESI(+) ionization mode; flow rate=1.2 mL/min. Mobile phase A: Water/5 mM NH₄HCO₃, Mobile phase B: CH₃CN.)

Analytical chiral HPLC: Analytical chiral HPLC was performed on a Agilent 1260 using column: CHIRALPAK IG-3, CHIRALPAK IC-3 or CHIIRALPAK OJ-3, with flow rate=1.2 mL/min. Mobile phase=MTBE(DEA):EtOH=50:50). Preparative HPLC purification: prep-HPLC purification was performed using one of the following HPLC conditions:

Condition 1: Waters-2545, Column: X-Select CSH (C18 OBD 130 Å, 5 μm, 30 mm×150 mm). Mobile Phase A: water (10 mmol/L NH₄HCO₃), Mobile Phase B: acetonitrile, Gradient:55% B to 50% B in 8 min.

Condition 2: Shimadzu, Column: XBridge Prep C18 OBD Column, 5 um, 19 mm×150 mm; Mobile Phase A: water (10 mmol/L NH₄HCO₃), Mobile Phase B: methanol, Gradient: 30% B up to 50% in 10 min.

Condition 3: Shimadzu, Column: Xselect CSH OBD Column, 30 mm×150 mm, Sum, Mobile Phase A: Water (10 mmol/L NH₄HCO₃), Mobile Phase B: acetonitrile, Gradient 1: 10% Phase B up to 60% in 8 min; Gradient 2: 5% Phase B up to 40% in 8 min

Condition 4: Shimadzu, Column: XBridge Prep OBD C18 Column, 30×150 mm, 5 μm; Mobile Phase A: water (10 mmol/L NH₄HCO₃), Mobile Phase B: acetonitrile; Gradient 1: 10 B to 44 B in 8 min; Gradient 2: 3 B to 33 B in 6 min; Gradient 3: 5 B to 35 B in 8 min; Gradient 4: 5 B to 24 B in 8 min; Gradient 5: 5 B to 43 B in 6 min; Gradient 6: hold 5 B in 2 min, up to 55 B in 6 min; Gradient 7: 5% B to 36% B in 8 min; Gradient 8: 10% B up to 65% B in 8 min; Gradient 9: 5% B to 32% B in 8 min; Gradient 10: 5% B to 50% B in 8 min.

Condition 5: Column: Xselect CSH OBD Column 30*150 mm Sum, n; Mobile Phase A: water (10 mmol/L NH₄HCO₃); Mobile Phase B: acetonitrile; Flow rate: 60 mL/min; Gradient: 10 B to 35 B in 10 min.

Condition 6: Column, Xselect CSH OBD Column 30×150 mm 5 um; Mobile Phase A: water (0.1% HCl); Mobile Phase B: acetonitrile; Gradient 1: Hold 3% phase B for 2 min, then ramp up to 23% over 6 min.

Condition 7: Column, YMC-Actus Triart C18, 30×150 mm, Sum; Mobile Phase A: water (10 mmol/L NH₄HCO₃); Mobile Phase B: acetonitrile; Gradient 1: 5% B to 50% B in 8 min; Gradient 2: 5% B to 45% B in 8 min; Gradient 3: 5% B to 65% B in 8 min; Gradient 4: 15% B to 50% B in 8 min.

Condition 8: Column: Xselect CSH OBD Column 30×150 mm 5 um; Mobile Phase A: water (0.05% HCl), Mobile Phase B: acetonitrile; Flow rate: 60 mL/min; Gradient 1: 3% B to 3% B in 2 min; Gradient 2: 3% B to 23% B in 8 min; Gradient 3: 5% B to 40% B in 8 min; Gradient 4: 3% B to 35% B in 8 min; Gradient 5: 3% B to 43% B in 8 min.

Condition 9: Column: XBridge Shield RP18 OBD Column, 19×150 mm, 5 m; Mobile Phase A: water (0.05% TFA), Mobile Phase B: acetonitrile; Flow rate: 20 mL/min; Gradient 1: 5% B to 22% B in 8 min.

Condition 10: Column: SunFire Prep C18 OBD Column, 19×150 mm, 5 m 10 nm; Mobile Phase A: water (0.05% TFA), Mobile Phase B: acetonitrile; Flow rate: 20 mL/min; Gradient 1: 10% B to 55% B in 6.9 min.

Condition 11: Column: XBridge Shield RP18 OBD Column, 19×150 mm, 5 m; Mobile Phase A: water (0.05% NH₃H₂O), Mobile Phase B: acetonitrile; Flow rate: 20 mL/min; Gradient 1: 36% B to 54% B in 8 min.

Preparative chiral HPLC: purification by chiral HPLC was performed on a Gilson-GX 281 using column: CHIRALPAK 1G-3, CHIRALPAK IC-3 or CHIRALPAK OJ-3.

Condition 1: Column, XBridge Prep OBD C18 Column, 30×150 mm; Mobile Phase A, water (10 mmol/L NH₄HCO₃), Mobile Phase B: acetonitrile; Gradient 1: 5% B to 50% B in 8 min.

### General Synthetic Scheme

Compounds of the present disclosure may be prepared using a synthetic protocol illustrated in any one of Schemes A-D.

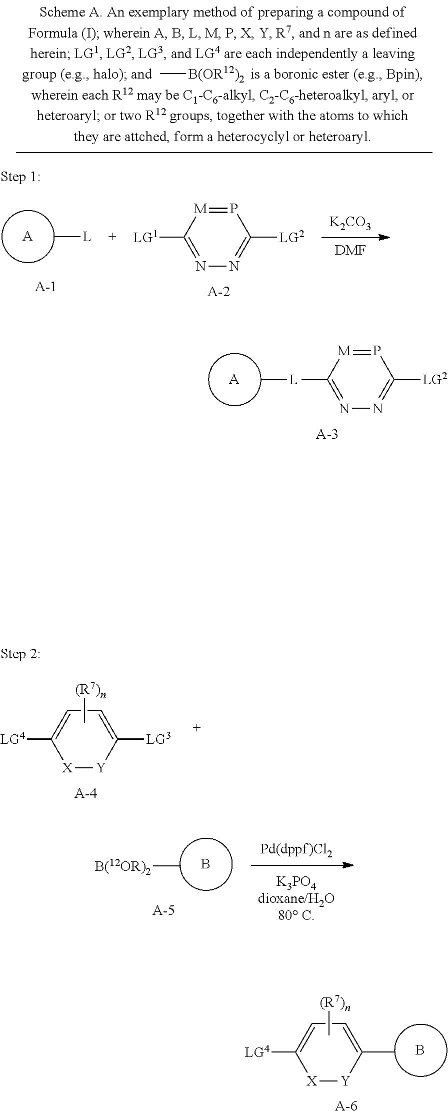

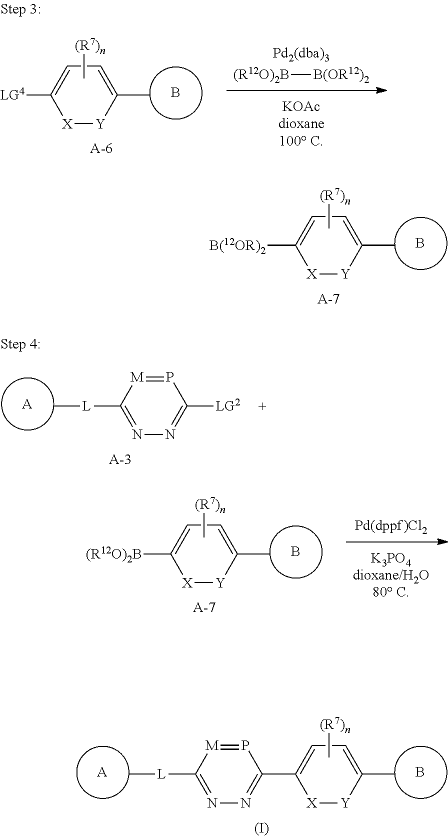

An exemplary method of preparing a compound of Formula (I) is provided in Scheme A. In this scheme, A-3 is prepared in Step 1 by incubating A-1 with A-2 in the presence of a base, for example, potassium carbonate (K₂CO₃) or sodium hydride (NaH) in N,N-dimethylformamide (DMF) or another suitable reagent. In some instances, A-3 is prepared by heating the reaction mixture to a suitable temperature, for example, 100° C. In Step 2, A-6 is prepared by incubating A-4 with A-5. Step 2 may be carried out in the presence of 1,1′-bis(diphenylphosphino)-ferrocene)palladium(II) dichloride (Pd(dppf)Cl₂), and tripotassium phosphate (K₃PO₄) or a similar reagent, for example, potassium carbonate (K₂CO₃). Alternative catalysts to Pd(dppf)Cl₂ may also be used, such as a suitable palladium catalyst (e.g., a catalyst suitable for a Suzuki reaction), for example, tetrakis(triphenylphosphine)-palladium(0) (Pd(PPh₃)₄). The coupling of A-4 and A-5 may be carried out in a mixture of dioxane and water, or a similar solvent or mixture, and heated to 80° C. or temperature sufficient to provide A-6, for example, 100° C.

In Step 3, A-7 is prepared by incubating A-6 with a reagent suitable to displace LG⁴ with a boronic ester group, such as (R¹²O)₂B—B(OR¹²)₂ (e.g., bis(pinacolato)diboron (B₂pin₂)). Other common reagents for installing boronic ester groups (e.g., pinacol borane) can also be used. This reaction may involve the use of tris(dibenzylideneacetone)dipalladium(0) (Pd₂(dba)₃), and potassium acetate (KOAc) or a suitable alternative, for example, tripotassium phosphate (K₃PO₄). Step 3 may also be carried out using an alternative catalyst to Pd₂(dba)₃, such as another palladium catalyst, for example, [1,1′-bis(di-tert-butylphosphino)ferrocene]dichloropalladium(II) (Pd(dtbpf)Cl₂) or chloro(2-dicyclohexylphosphino-2′, 4′, 6′-triisopropyl-1,1′-biphenyl)[2-(2′-amino-1,1′-biphenyl)]palladium(II) (XPhos-Pd-G2). The reaction may be conducted in dioxane or a similar solvent, at 100° C. or a temperature sufficient to provide Fragment A-7, for example, 80° C., 90° C., 110° C., or 120° C. The reaction may be conducted in a microwave reactor.

A-3 and A-7 are coupled to provide a compound of Formula (I) in Step 4. This coupling reaction may be conducted in the presence of Pd(dppf)Cl₂, and K₃PO₄ or a similar reagent, for example tripotassium carbonate (K₃PO₄). As in Step 2, alternative catalysts to Pd(dppf)Cl₂ may be used, such as any suitable palladium catalyst, for example, tetrakis(triphenylphosphine)-palladium(0) (Pd(PPh₃)₄) or chloro(2-dicyclohexylphosphino-2′, 4′, 6′-triisopropyl-1,1′-biphenyl)[2-(2′-amino-1,1′-biphenyl)]palladium(II) (XPhos-Pd-G2). The reaction of Step 4 is conducted in dioxane or a mixture of dioxane and water, or other suitable solvents, and the mixture is heated to 80° C. or a temperature sufficient to provide the compound of Formula (I) or a precursor to the compound of Formula (I) with one or more protecting group(s), for example, 100° C. Compounds of Formula (I) may be purified using standard techniques and characterized using any method known in the art, such as nuclear magnetic resonance spectroscopy (NMR) or mass spectrometry (MS).

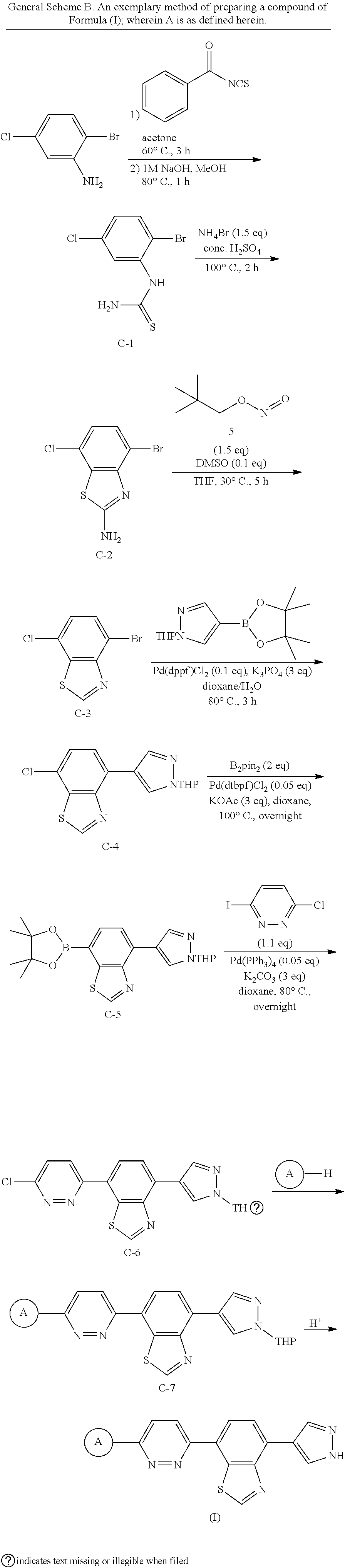

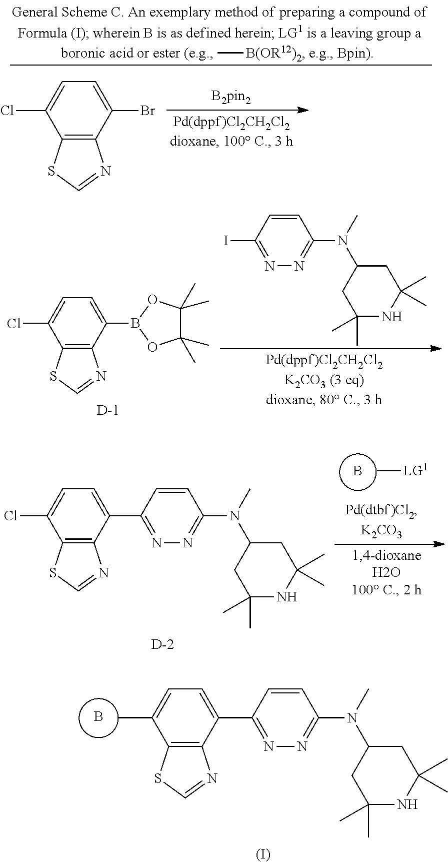

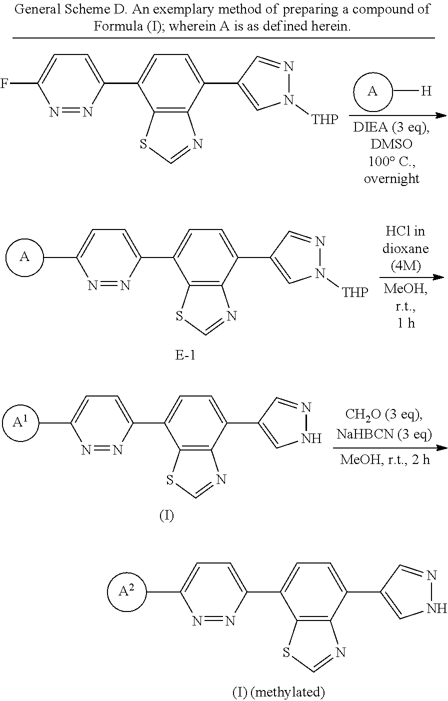

### Example 1: Synthesis of Compound 101

### Synthesis of Intermediate B2

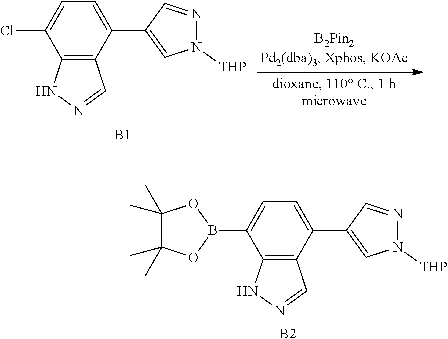

A mixture of 7-chloro-4-[1-(oxan-2-yl)pyrazol-4-yl]-1H-indazole (B1; 500 mg, 1.65 mmol, 1 equiv), B₂Pin₂ (838 mg, 3.30 mmol, 2 equiv), XPhos (78 mg, 0.17 mmol, 0.1 equiv), KOAc (486 mg, 4.95 mmol, 3 equiv) and Pd₂(dba)₃-CHCl₃ (85 mg, 0.08 mmol, 0.05 equiv) in dioxane (15.0 mL) was stirred for 2h at 120° C. in a 30 mL microwave tube, using a microwave reactor. The reaction was quenched with H₂O at 0° C. The aqueous layer was extracted with ethyl acetate (EA) (50 mL×3), and the combined organic layers were washed with brine (50 mL), and then dried with Na₂SO₄. The resulting mixture was filtered, and the filter cake was washed with EA (50 mL×2). The filtrate was concentrated under reduced pressure to provide 4-[1-(oxan-2-yl)pyrazol-4-yl]-7-(4,4,5,5-tetramethyl-1,3,2-dioxaborolan-2-yl)-1H-indazole (B2; 600 mg) as a solid. LCMS (ES, m/z): 395[M+H]⁺.

### Synthesis of Intermediate B4

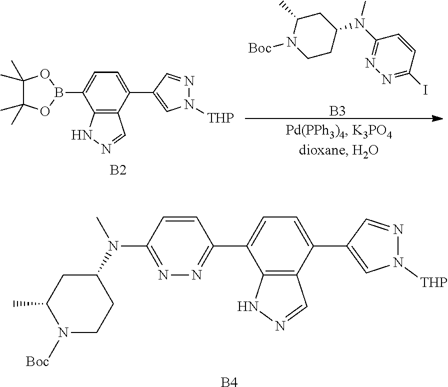

A mixture of 4-[1-(oxan-2-yl)pyrazol-4-yl]-7-(4,4,5,5-tetramethyl-1,3,2-dioxaborolan-2-yl)-1H-indazole (B2; 500 mg, 1.27 mmol, 1 equiv), (cis)-tert-butyl-(2R,4R)-4-[(6-iodopyridazin-3-yl)(methyl)amino]-2-methylpiperidine-1-carboxylate (B3; 822 mg, 1.90 mmol, 1.5 equiv), Pd(PPh₃)₄ (146 mg, 0.13 mmol, 0.1 equiv) and K₃PO₄ (807 mg, 3.81 mmol, 3 equiv) in dioxane (10.0 mL) and H₂O (5.0 mL) was stirred for 3h at 100° C. under a nitrogen atmosphere. The reaction was quenched with H₂O at 0° C. The aqueous layer was extracted with ethyl acetate (EA) (50 mL×3), and the combined organic layer was washed with NaCl (aq), and then dried with Na₂SO₄. The residue was purified by silica gel column chromatography, eluting with petroleum ether (PE): ethyl acetate (EA) (1:2). The resulting mixture was concentrated under reduced pressure to provide tert-butyl (2R,4R)-2-methyl-4-[methyl(6-[4-[1-(oxan-2-yl)pyrazol-4-yl]-1H-indazol-7-yl]pyridazin-3-yl)amino]piperidine-1-carboxylate (B4; 256 mg) as a solid. LCMS (ES, m/z): 573[M+H]⁺.

### Synthesis of Compound 101

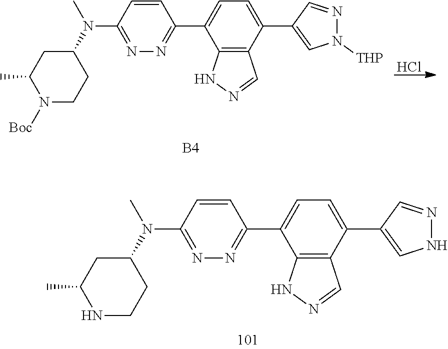

A mixture of (cis)-tert-butyl (2R,4R)-2-methyl-4-[methyl(6-[4-[1-(oxan-2-yl)pyrazol-4-yl]-1H-indazol-7-yl]pyridazin-3-yl)amino]piperidine-1-carboxylate (B4; 256 mg, 0.45 mmol, 1 equiv), 4M HCl in 1,4-dioxane (5 mL), dioxane (5 mL), and MeOH (3 mL), was stirred for 2h at 0° C. under a nitrogen atmosphere in a purged 50 mL 3-necked round-bottom flask. The resulting mixture was concentrated under reduced pressure, and then stirred in NH₃/MeOH for 3h. The resulting mixture was filtered, and the filter cake was washed with NH₃/MeOH. The filtrate was concentrated under reduced pressure to provide (cis)-N-methyl-N-[(2R,4R)-2-methylpiperidin-4-yl]-6-[4-(1H-prazol-4-yl)-1H-indazol-7-yl]pyridazin-3-amine (Compound 101; 21 mg) as a solid. LCMS (ES, m/z): 389[M+H]⁺. ¹H NMR (400 MHz, Methanol-d₄) δ 8.45 (s, 2H), 8.33 (s, 2H), 8.19 (d, J=9.8 Hz, 3H), 7.94 (d, J=7.6 Hz, 2H), 7.51 (d, J=7.6 Hz, 2H), 7.35 (d, J=9.7 Hz, 2H), 4.99 (s, 1H), 4.61 (s, 1H), 3.59 (dt, J=13.0, 3.4 Hz, 2H), 3.52-3.43 (m, 1H), 3.47 (s, 1H), 3.37 (s, 1H), 3.25 (dd, J=13.5, 8.2 Hz, 2H), 3.10 (s, 6H), 2.19-2.07 (m, 5H), 2.07 (d, J=3.6 Hz, 1H), 1.92 (q, J=12.4 Hz, 2H), 1.44 (d, J=6.4 Hz, 6H), 0.12 (s, 2H).

### Example 2: Synthesis of Compound 103

### Synthesis of Intermediate B6

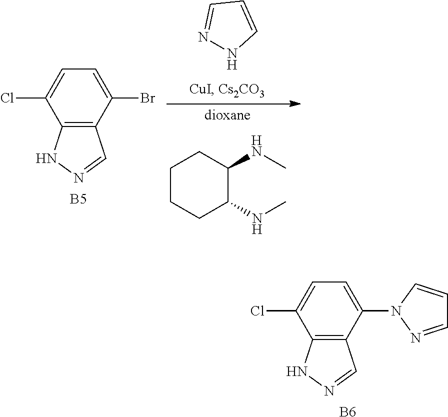

A 100-mL sealed tube was purged and maintained under an atmosphere of nitrogen, and 4-bromo-7-chloro-1H-indazole (B5; 2.7 g, 11.66 mmol, 1 equiv), pyrazole (3.18 g, 46.64 mmol, 4 equiv), Cs₂CO₃ (11.40 g, 34.99 mmol, 3 equiv), dioxane (60 mL), CuI (0.33 g, 1.75 mmol, 0.15 equiv), (1S,2S)—N1,N2-dimethylcyclohexane-1,2-diamine (0.41 g, 2.92 mmol, 0.25 equiv) were added to the tube. The resulting solution was stirred for 7 days at 100° C., after which the solids were removed by filtration. The filtrate was then extracted with ethyl acetate (3×100 mL), and the combined organic layers were washed with saturated NaCl (100 mL). The mixture was then dried over anhydrous sodium sulfate and concentrated under vacuum. The residue was purified by silica gel column chromatography, eluting with ethyl acetate/petroleum ether (1:5), to provide 7-chloro-4-(pyrazol-1-yl)-1H-indazole (B6; 1 g) as a solid. LCMS (ES, m/z): 219 [M+H]⁺.

### Synthesis of Intermediate B7

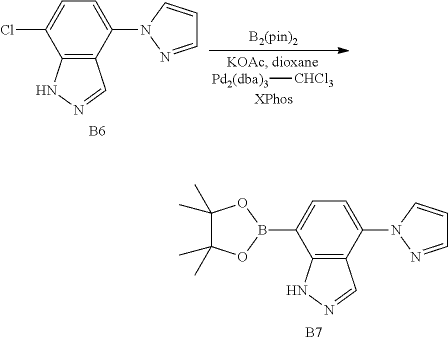

A 20-mL vial was purged and maintained under a nitrogen atmosphere, and 7-chloro-4-(pyrazol-1-yl)-1H-indazole (B6; 1 g, 4.57 mmol, 1 equiv), KOAc (1.35 mg, 0.01 mmol, 3 equiv), B₂ (pin)₂ (2.56 mg, 0.01 mmol, 2.20 equiv), dioxane (10 mL), Pd₂(dba)₃-CHCl₃ (0.47 mg, 0.01 mmol, 0.10 equiv), and XPhos (0.87 mg, 0.01 mmol, 0.40 equiv) were added to the vial. The resulting solution was stirred for 2 hr at 120° C., and the solids were removed by filtration. The resulting solution was then extracted with ethyl acetate (3×20 mL), and the organic layers were combined to provide 4-(pyrazol-1-yl)-7-(4,4,5,5-tetramethyl-1,3,2-dioxaborolan-2-yl)-1H-indazole (B7; 1 g) as an oil. LCMS (ES, m/z): 311 [M+H]⁺.

### Synthesis of Intermediate B8

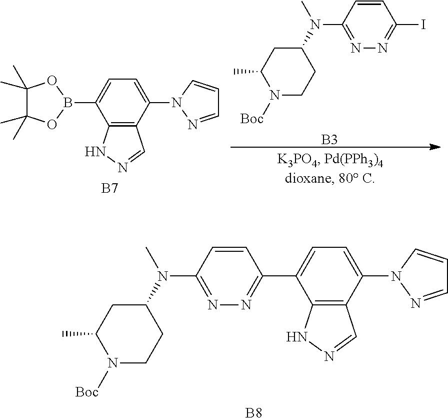

A 40-mL vial was purged and maintained under an atmosphere of nitrogen, and 4-(pyrazol-1-yl)-7-(4,4,5,5-tetramethyl-1,3,2-dioxaborolan-2-yl)-1H-indazole (B7; 500 mg, 1.61 mmol, 1 equiv), (cis)-tert-butyl-4-[(6-iodopyridazin-3-yl)(methyl)amino]-2-methylpiperidine-1-carboxylate (B3; 696 mg, 1.61 mmol, 1 equiv), dioxane (20 mL), K₃PO₄ (1026 mg, 4.84 mmol, 3 equiv), and Pd(PPh₃)₄ (93 mg, 0.08 mmol, 0.05 equiv) were added to the vial. The resulting solution was stirred for 7 hr at 80° C., and the solids were then removed by filtration. The resulting solution was extracted with ethyl acetate (3×20 mL), and the combined organic layers were concentrated under vacuum. The residue was purified by silica gel column chromatography, eluting with ethyl acetate/petroleum ether (7:10), to provide (cis)-tert-butyl-2-methyl-4-[methyl([6-[4-(pyrazol-1-yl)-1H-indazol-7-yl]pyridazin-3-yl])amino]piperidine-1-carboxylate (B8; 300 mg) as a solid. LCMS (ES, m/z): 489 [M+H]⁺.

### Synthesis of Compound 103

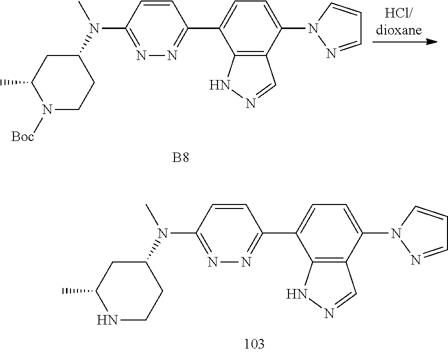

A 100-mL round-bottom flask was purged and maintained under an atmosphere of argon, and (cis)-tert-butyl(2S,4S)-2-methyl-4-[methyl([6-[4-(pyrazol-1-yl)-1H-indazol-7-yl]pyridazin-3-yl])amino]piperidine-1-carboxylate (B8; 300 mg, 0.61 mmol, 1 equiv), and HCl/dioxane (30 mL) were added to the flask. The resulting solution was stirred for 2 hr at room temperature, and then concentrated under vacuum. The residue was dissolved in 5 mL of methanol, and the solids were removed by filtration. The crude product was purified by preparative HPLC (Condition 1), to provide N-methyl-N-[(2S,4S)-2-methylpiperidin-4-yl]-6-[4-(pyrazol-1-yl)-1H-indazol-7-yl]pyridazin-3-amine (Compound 103; 96.3 mg) as a solid. LCMS (ES, m/z): 389 [M+H]⁺. ¹H NMR (400 MHz, DMSO-d₆) δ 13.29 (s, 1H), 8.71 (d, J=2.6 Hz, 1H), 8.63 (s, 1H), 8.20 (d, J=9.8 Hz, 1H), 8.00 (d, J=8.0 Hz, 1H), 7.92 (d, J=1.7 Hz, 1H), 7.62 (d, J=8.0 Hz, 1H), 7.30 (d, J=9.8 Hz, 1H), 6.66 (t, J=2.2 Hz, 1H), 4.71 (s, 1H), 3.10-2.97 (m, 1H), 3.02 (s, 3H), 2.75-2.63 (m, 2H), 2.48 (d, J=7.3 Hz, 1H), 2.08 (s, 1H), 1.66 (qd, J=11.6, 11.0, 4.0 Hz, 2H), 1.60 (s, 1H), 1.35 (q, J=11.5 Hz, 1H), 1.05 (d, J=6.2 Hz, 3H).

### Example 3: Synthesis of Compound 104

### Synthesis of Intermediate B1

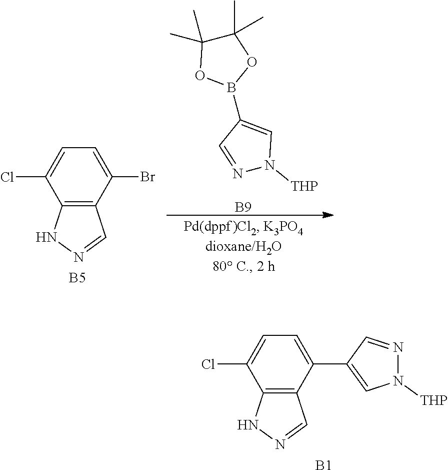

A mixture of K₃PO₄ (5.50 g, 25.920 mmol, 3 equiv), 1-(oxan-2-yl)-4-(4,4,5,5-tetramethyl-1,3,2-dioxaborolan-2-yl)pyrazole (B9; 3.12 g, 11.232 mmol, 1.3 equiv), 4-bromo-7-chloro-1H-indazole (B5; 2.0 g, 8.640 mmol, 1.0 equiv) and Pd(dppf)Cl₂—CH₂Cl₂ (352 mg, 0.432 mmol, 0.05 equiv) in dioxane (8.0 mL) and H₂O (2.0 mL) was stirred for 2h at 80° C. with deoxygenation. The resulting mixture was then concentrated under reduced pressure. The residue was purified by silica gel column chromatography, eluting with petroleum ether/ethyl acetate (4:1) to afford 7-chloro-4-[1-(oxan-2-yl)pyrazol-4-yl]-1H-indazole (B1; 1.8 g) as a solid. LCMS (ES, m/z): 302 [M+H]⁺.

### Synthesis of Intermediate B11

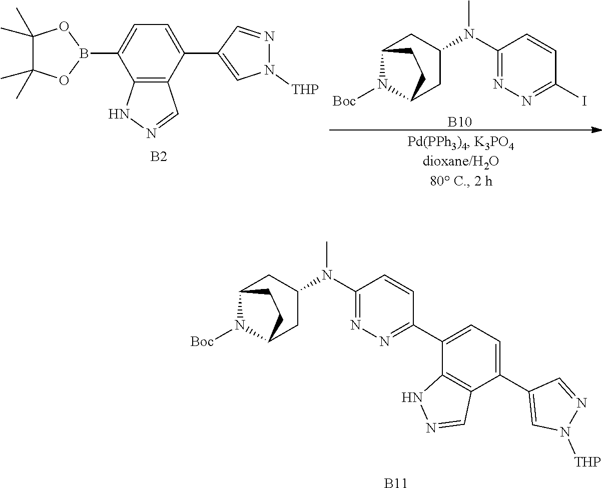

A mixture of K₃PO₄ (807.55 mg, 3.80 mmol, 3 equiv), tert-butyl-(1R,3S,5S)-3-[(6-iodopyridazin-3-yl)(methyl)amino]-8-azabicyclo[3.2.1]octane-8-carboxylate (B10; 845 mg, 1.9 mmol, 1.5 equiv) and 4-[1-(oxan-2-yl)pyrazol-4-yl]-7-(4,4,5,5-tetramethyl-1,3,2-dioxaborolan-2-yl)-1H-indazole (B2 from Example 1; 500 mg, 1.27 mmol, 1 equiv) in dioxane (16.0 mL) and H₂O (4.0 mL, 222.03 mmol) was stirred for 2h at 80° C. with deoxygenation. The reaction was quenched with H₂O (50 mL) at 0° C. The precipitated solids were collected by filtration and washed with ethyl acetate (50 mL×3). The resulting mixture was then washed with saturated aqueous NaCl (50 mL), and the organic phase was collected and dried with Na₂SO₄. The resulting mixture was concentrated under reduced pressure. The residue was purified by silica gel column chromatography, eluting with petroleum ether/ethyl acetate (1:2) to afford tert-butyl (1R,3S,5S)-3-[methyl(6-[4-[1-(oxan-2-yl)pyrazol-4-yl]-1H-indazol-7-yl]pyridazin-3-yl)amino]-8-azabicyclo[3.2.1]octane-8-carboxylate (B6; 480 mg) as a solid. LCMS (ES, m/z): 584 [M+H]⁺.

### Synthesis of Compound 104

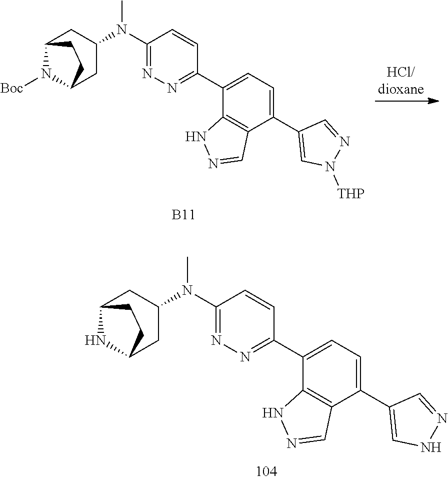

A mixture of tert-butyl-(1R,3S,5S)-3-[methyl(6-[4-[1-(oxan-2-yl)pyrazol-4-yl]-1H-indazol-7-yl]pyridazin-3-yl)amino]-8-azabicyclo[3.2.1]octane-8-carboxylate (B11; 480 mg), dioxane (5 mL), HCl in 1,4-dioxane (5 mL), and H₂O (3 mL) was stirred for 2h at 0° C. under a nitrogen atmosphere in a purged 250 mL 3-necked round-bottom flask. The resulting mixture was then concentrated under reduced pressure. The precipitated solids were collected by filtration and washed with acetonitrile (10 mL×2). The crude precipitate was then added to a purged 100 mL 3-necked round-bottom flask with NH₃-MeOH, and the mixture was stirred for 2h. The resulting mixture was concentrated under reduced pressure, and the precipitated solids were collected by filtration to provide (1R,3S,5S)—N-methyl-N-[6-[4-(1H-pyrazol-4-yl)-1H-indazol-7-yl]pyridazin-3-yl]-8-azabicyclo[3.2.1]octan-3-amine (Compound 104; 129.5 mg) as a solid. LCMS (ES, m/z): 400 [M+H]⁺. 1H NMR (400 MHz, DMSO-d₆) δ 13.21 (s, 1H), 13.08 (s, 1H), 9.13 (s, 2H), 8.52 (s, 1H), 8.34 (s, 2H), 8.20 (d, J=9.8 Hz, 1H), 7.92 (d, J=7.7 Hz, 1H), 7.48 (d, J=7.6 Hz, 1H), 7.34 (d, J=9.8 Hz, 1H), 5.21 (td, J=11.8, 5.9 Hz, 1H), 4.10 (s, 2H), 3.04 (s, 3H), 2.54-2.48 (m, 3H), 2.38-2.26 (m, 2H), 2.06 (s, 3H), 2.06 (d, J=16.4 Hz, 1H), 1.78 (ddd, J=13.9, 5.8, 2.7 Hz, 2H).

### Example 4: Synthesis of Compound 105

### Synthesis of Intermediate B13

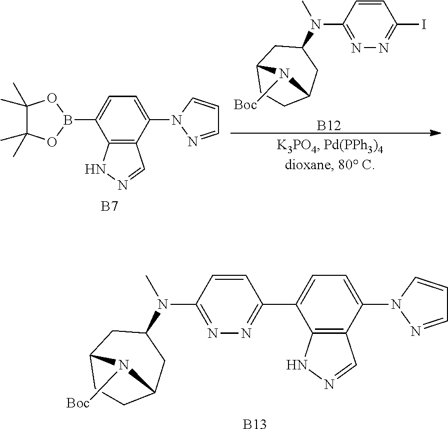

A 40-mL vial was purged and maintained under an atmosphere of nitrogen, and 4-(pyrazol-1-yl)-7-(4,4,5,5-tetramethyl-1,3,2-dioxaborolan-2-yl)-1H-indazole (B7; 500 mg, 1.61 mmol, 1 equiv), tert-butyl (1R,3R,5S)-3-[(6-iodopyridazin-3-yl)(methyl)amino]-8-azabicyclo[3.2.1]octane-8-carboxylate (B12; 716 mg, 1.61 mmol, 1 equiv), K₃PO₄ (1026 mg, 4.84 mmol, 3 equiv), dioxane (20 mL), and Pd(PPh₃)₄ (93 mg, 0.08 mmol, 0.05 equiv) were added to the vial. The resulting solution was stirred for 7 hr at 80° C., after which the solids were removed by filtration. The resulting solution was extracted with ethyl acetate (3×20 mL), and the resulting mixture was washed with saturated NaCl (20 mL), dried over anhydrous sodium sulfate, and then concentrated under vacuum. The residue was purified by silica gel column chromatography, eluting with ethyl acetate/petroleum ether (7:10), to provide tert-butyl (1R,3R,5S)-3-[methyl([6-[4-(pyrazol-1-yl)-1H-indazol-7-yl]pyridazin-3-yl])amino]-8-azabicyclo[3.2.1]octane-8-carboxylate (B13; 450 mg) as a solid.

### Synthesis of Compound 103

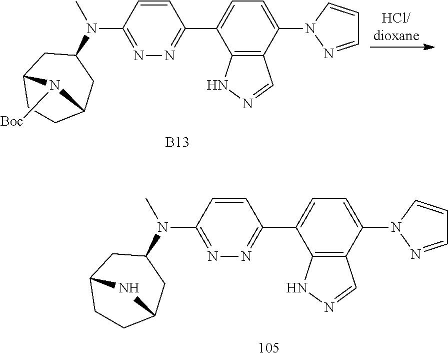

tert-Butyl (1R,3R,5S)-3-[methyl([6-[4-(pyrazol-1-yl)-1H-indazol-7-yl]pyridazin-3-yl])amino]-8-azabicyclo[3.2.1]octane-8-carboxylate (B13; 425 mg, 0.85 mmol, 1 equiv) and HCl/dioxane (100 mL) were stirred in a 100 mL round-bottom flask for 2h at room temperature. The resulting mixture was concentrated under vacuum. The residue was then dissolved in methanol (5 mL), and the solids were removed by filtration. The crude product was purified by preparative HPLC to provide (1R,3R,5S)—N-methyl-N-[6-[4-(pyrazol-1-yl)-1H-indazol-7-yl]pyridazin-3-yl]-8-azabicyclo[3.2.1]octan-3-amine (Compound 105; 34.90 mg) as a solid. LCMS (ES, m/z): 401 [M+H]⁺. 1H NMR (400 MHz, DMSO-d₆) δ 13.29 (s, 1H), 8.71 (d, J=2.6 Hz, 1H), 8.64 (s, 1H), 8.21 (d, J=9.8 Hz, 1H), 8.01 (d, J=8.1 Hz, 1H), 7.92 (d, J=1.7 Hz, 1H), 7.62 (d, J=7.9 Hz, 1H), 7.32 (d, J=9.7 Hz, 1H), 6.67 (t, J=2.2 Hz, 1H), 5.14 (s, 1H), 3.82 (s, 2H), 3.01 (s, 3H), 2.47 (s, 2H), 2.04 (td, J=12.6, 3.0 Hz, 2H), 1.92 (s, 4H), 1.69 (ddd, J=13.2, 6.1, 2.7 Hz, 2H).

### Example 5: Synthesis of Compounds 107 and 108

### Synthesis of Intermediate B15

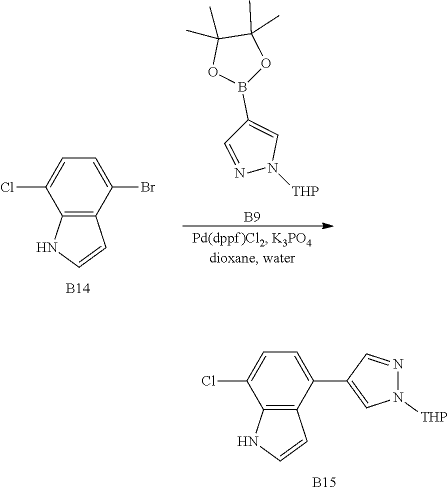

A 40-mL round-bottom flask was purged and maintained under an atmosphere of nitrogen, and 4-bromo-7-chloro-1H-indole (B14; 1.5 g, 6.51 mmol, 1.0 equiv), 1-(oxan-2-yl)-4-(4,4,5,5-tetramethyl-1,3,2-dioxaborolan-2-yl)pyrazole (B9; 2.17 g, 7.81 mmol, 1.2 equiv), Pd(dppf)Cl₂ (0.24 g, 0.33 mmol, 0.05 equiv), K₃PO₄ (4.14 g, 19.51 mmol, 3.0 equiv), dioxane (10.0 mL), and H₂O (3.0 mL) were added to the flask. The resulting solution was stirred for 2h at 80° C. The reaction was then quenched by the addition of ice/salt (10 mL). The resulting solution was extracted with ethyl acetate (3×10 mL), and the combined organic layers were dried over anhydrous sodium sulfate, and then concentrated. The residue was purified by silica gel column chromatography, eluting with ethyl acetate/hexanes (1:1), to provide 7-chloro-4-[1-(oxan-2-yl)pyrazol-4-yl]-1H-indole (B15; 1.5 g) as a solid. LCMS (ES, m/z): 302 [M+H]⁺

### Synthesis of Intermediate B16

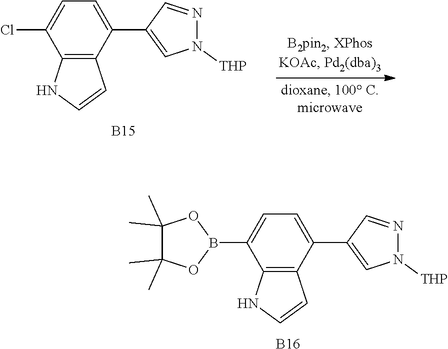

A 20-mL sealed tube was purged and maintained with under an atmosphere of nitrogen, and 7-chloro-4-[1-(oxan-2-yl)pyrazol-4-yl]-1H-indole (B15; 1.0 g, 3.31 mmol, 1.0 equiv), bis(pinacolato)diboron (1.51 g, 5.97 mmol, 1.8 equiv), potassium acetate (0.98 g, 9.94 mmol, 3.0 equiv), Pd₂(dba)₃ (0.24 g, 0.265 mmol, 0.08 equiv), XPhos (0.47 g, 0.99 mmol, 0.3 equiv), and dioxane (10.0 mL) were added to the tube. The reaction mixture was irradiated with microwave radiation for 2h at 110° C. The reaction was then quenched by the addition of water/ice (10 mL). The resulting solution was extracted with ethyl acetate (2×10 mL) and the combined organic layers were concentrated to provide 4-[1-(oxan-2-yl)pyrazol-4-yl]-7-(4,4,5,5-tetramethyl-1,3,2-dioxaborolan-2-yl)-1H-indole (B16; 1.1 g) as an oil. LCMS (ES, m/z): 394 [M+H]⁺

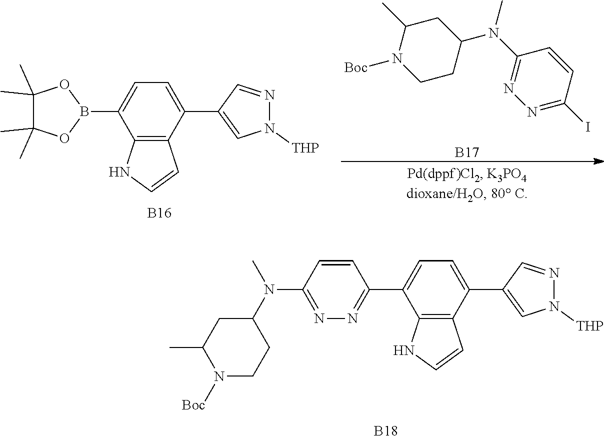

A 40 mL vial was purged and maintained under an atmosphere of nitrogen, and 4-[1-(oxan-2-yl)pyrazol-4-yl]-7-(4,4,5,5-tetramethyl-1,3,2-dioxaborolan-2-yl)-1H-indole (B16; 1.1 g, 2.79 mmol, 1.0 equiv), tert-butyl 4-[(6-iodopyridazin-3-yl)(methyl)amino]-2-methylpiperidine-1-carboxylate (B17; 1.81 g, 4.19 mmol, 1.5 equiv), Pd(dppf)Cl₂ (0.1 g, 0.14 mmol, 0.05 equiv), K₃PO₄ (1.78 g, 8.39 mmol, 3.0 equiv), dioxane (10.0 mL), and H₂O (2.5 mL) were added to the vial. The resulting solution was stirred for 3h at 80° C., and the reaction was quenched by the addition of water/ice (10 mL). The resulting solution was extracted with ethyl acetate (3×10 mL), and the combined organic layers were dried over anhydrous sodium sulfate and then concentrated. The residue was purified by silica gel column chromatography eluting with ethyl acetate/hexanes (1:1), to provide tert-butyl 2-methyl-4-[methyl(6-[4-[1-(oxan-2-yl)pyrazol-4-yl]-1H-indol-7-yl]pyridazin-3-yl)amino]piperidine-1-carboxylate (B18; 500 mg) as a solid. LCMS (ES, m/z): 572 [M+H]⁺

### Synthesis of Compound 107 and 108

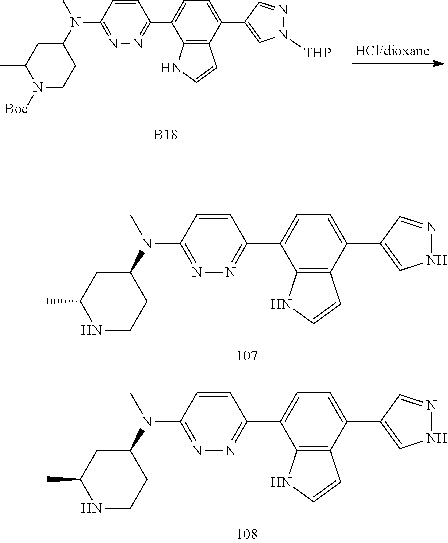

A 10 mL round-bottom flask was purged and maintained under an atmosphere of nitrogen, and tert-butyl 2-methyl-4-[methyl(6-[4-[1-(oxan-2-yl)pyrazol-4-yl]-1H-indol-7-yl]pyridazin-3-yl)amino]piperidine-1-carboxylate (B18; 500 mg), and 4M HCl in 1,4-dioxane (2.0 mL) were added to the flask. The resulting solution was stirred for 1h at 25° C., and the resulting mixture was concentrated. The crude product was purified by preparative HPLC (Condition 2), to provide N-methyl-N-[(2R,4S)-2-methylpiperidin-4-yl]-6-[4-(1H-pyrazol-4-yl)-1H-indol-7-yl]pyridazin-3-amine (Compound 107; 4.7 mg) as a solid. LCMS (ES, m/z): 388 [M+H]⁺. ¹H NMR (400 MHz, DMSO-d₆) δ 11.58 (s, 1H), 8.15-8.21 (m, 3H), 7.69 (d, J=7.9 Hz, 1H), 7.47 (t, J=2.8 Hz, 1H), 7.34 (d, J=7.8 Hz, 1H), 7.26 (d, J=9.8 Hz, 1H), 6.75-6.89 (m, 1H), 4.62-4.69 (m, 1H), 2.99-3.09 (m, 1H), 3.00 (s, 3H), 2.67-2.70 (m, 2H), 1.63-1.70 (m, 3H), 1.27-1.40 (m, 1H), 1.03-1.05 (m, 3H); and N-methyl-N-[(2S,4S)-2-methylpiperidin-4-yl]-6-[4-(1H-pyrazol-4-yl)-1H-indol-7-yl]pyridazin-3-amine (Compound 108; 4.7 mg) as a solid. LCMS (ES, m/z): 388 [M+H]⁺. 1H NMR (400 MHz, DMSO-d₆) δ 13.06 (s, 1H), 11.56 (s, 1H), 8.14-8.17 (m, 3H), 7.68 (d, J=7.8 Hz, 1H), 7.48 (t, J=2.9 Hz, 1H), 7.26-7.41 (m, 2H), 6.83 (t, J=2.7 Hz, 1H), 4.88 (s, 1H), 3.31-3.32 (m, 1H), 3.01 (s, 3H), 2.90-3.00 (m, 1H), 2.81-2.83 (m, 1H), 1.93 (td, J=12.2, 5.2 Hz, 1H), 1.63-1.75 (m, 2H), 1.45-1.54 (m, 1H), 1.23-1.26 (m, 3H).

### Example 6: Synthesis of Compounds 110 and 111

### Synthesis of Intermediate B19

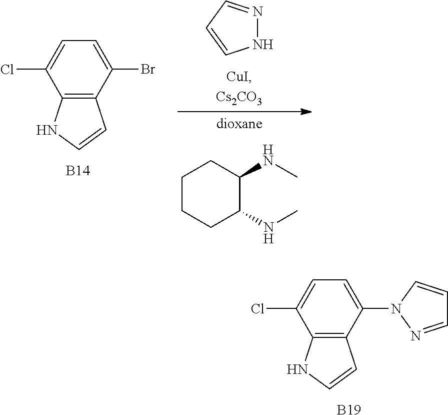

A 40-mL vial was purged and maintained under an atmosphere of nitrogen, and 4-bromo-7-chloro-1H-indole (B14; 2.0 g, 8.68 mmol, 1.0 equiv), pyrazole (1.77 g, 26.03 mmol, 3.0 equiv), (1R,2S)—N1,N2-dimethylcyclohexane-1,2-diamine (0.25 g, 1.73 mmol, 0.2 equiv), CuI (0.66 g, 3.47 mmol, 0.4 equiv), Cs₂CO₃ (8.48 g, 26.03 mmol, 3.0 equiv), and dioxane (20.0 mL) were added to the vial. The resulting solution was stirred for 7 days at 100° C., and the reaction was quenched by the addition of water/ice (10 mL). The resulting solution was extracted with ethyl acetate (3×10 mL), and the combined organic layers were dried over anhydrous sodium sulfate, and then concentrated. The residue was purified by silica gel column chromatography, eluting with dichloromethane/methanol (10:1), to provide 7-chloro-4-(pyrazol-1-yl)-1H-indole (B19; 1.2 g) as a solid. LCMS (ES, m/z): 218 [M+H]⁺.

### Synthesis of Intermediate B20

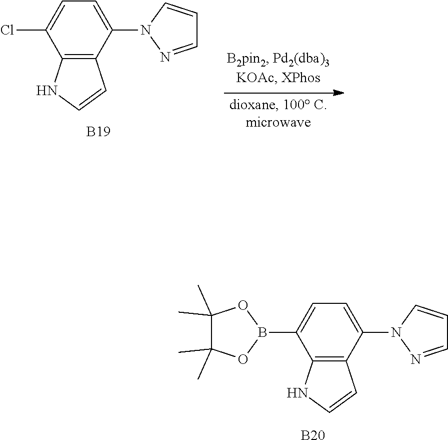

A 20 mL sealed tube was purged and maintained under an atmosphere of nitrogen, and 7-chloro-4-(pyrazol-1-yl)-1H-indole (B19; 1.2 g, 5.51 mmol, 1.0 equiv), bis(pinacolato)diboron (2.52 g, 9.92 mmol, 1.8 equiv), potassium acetate (1.62 g, 16.54 mmol, 3.0 equiv), Pd₂(dba)₃ (0.4 g, 0.44 mmol, 0.08 equiv), XPhos (0.79 g, 1.65 mmol, 0.3 equiv), and 1,4-dioxane (12.0 mL) were added to the tube. The reaction mixture was then irradiated with microwave radiation for 2h at 110° C., and the reaction was quenched by the addition of water/ice (10 mL). The resulting solution was then extracted with ethyl acetate (3×10 mL), and the combined organic layers were concentrated to provide 4-(pyrazol-1-yl)-7-(4,4,5,5-tetramethyl-1,3,2-dioxaborolan-2-yl)-1H-indole (B20; 1.2 g) as an oil. LCMS (ES, m/z): 310 [M+H]⁺.

### Synthesis of Intermediate B21

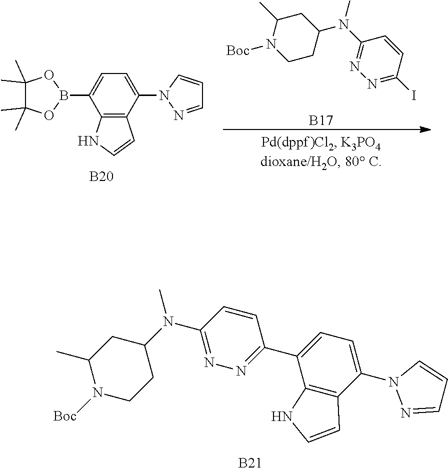

A 10 mL round-bottom flask was purged and maintained under an atmosphere of nitrogen, and 4-(pyrazol-1-yl)-7-(4,4,5,5-tetramethyl-1,3,2-dioxaborolan-2-yl)-1H-indole (B20; 500 mg, 1.61 mmol, 1.0 equiv), tert-butyl 4-[(6-iodopyridazin-3-yl)(methyl)amino]-2-methylpiperidine-1-carboxylate (B17; 908 mg, 2.11 mmol, 1.3 equiv), Pd(dppf)Cl₂ (59 mg, 0.081 mmol, 0.05 equiv), K₃PO₄ (1029 mg, 4.85 mmol, 3.0 equiv), dioxane (4.0 mL), and H₂O (1 mL) were added to the flask. The resulting solution was stirred for 3h at 80° C., and the reaction was quenched by the addition of ice/salt (10 mL). The resulting solution was extracted with ethyl acetate (10 mL), and the combined organic layers were dried over anhydrous sodium sulfate and concentrated. The residue was purified by silica gel column chromatography eluting with dichloromethane/methanol (10:1), to provide tert-butyl 2-methyl-4-[methyl([6-[4-(pyrazol-1-yl)-1H-indol-7-yl]pyridazin-3-yl])amino]piperidine-1-carboxylate (B21; 700 mg) as a solid. LCMS (ES, m/z): 488 [M+H]⁺.

### Synthesis of Compounds 110 and 111

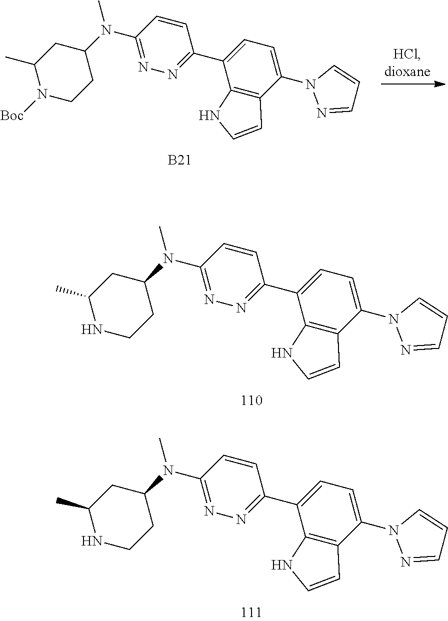

A 25-mL round-bottom flask was purged and maintained under an atmosphere of nitrogen, and tert-butyl 2-methyl-4-[methyl([6-[4-(pyrazol-1-yl)-1H-indol-7-yl]pyridazin-3-yl])amino]piperidine-1-carboxylate (B21; 700.0 mg), and 4M HCl in 1,4-dioxane (5 mL) were added to the flask. The resulting solution was stirred for 2h at 25° C., and then concentrated. The crude product was purified by preparative HPLC (Condition 3), to provide N-methyl-N-[(2R,4S)-2-methylpiperidin-4-yl]-6-[4-(pyrazol-1-yl)-1H-indol-7-yl]pyridazin-3-amine (Compound 110; 28.1 mg) as a solid; LCMS (ES, m/z): 388 [M+H]⁺. ¹H NMR (400 MHz, DMSO-d₆) δ 11.74 (s, 1H), 8.47 (d, J=2.5 Hz, 1H), 8.18 (d, J=9.8 Hz, 1H), 7.84 (d, J=1.8 Hz, 1H), 7.78 (d, J=8.2 Hz, 1H), 7.52 (t, J=2.9 Hz, 1H), 7.45 (d, J=8.1 Hz, 1H), 7.29 (d, J=9.9 Hz, 1H), 6.95 (t, J=2.7 Hz, 1H), 6.60 (t, J=2.1 Hz, 1H), 4.91 (s, 1H), 3.31-3.33 (m, 1H), 3.00-3.03 (m, 4H), 2.85-2.88 (m, 1H), 1.96-1.99 (m, 1H), 1.67-1.70 (m, 2H), 1.45-1.57 (m, 1H), 1.26 (d, J=6.9 Hz, 3H); and N-methyl-N-[(2R,4S)-2-methylpiperidin-4-yl]-6-[4-(pyrazol-1-yl)-1H-indol-7-yl]pyridazin-3-amine (Compound 111; 66.1 mg) as a solid. LCMS (ES, m/z): 388 [M+H]⁺. 1H NMR (400 MHz, DMSO-d₆) δ 11.74 (s, 1H), 8.47 (d, J=2.5 Hz, 1H), 8.18 (d, J=9.8 Hz, 1H), 7.84 (d, J=1.8 Hz, 1H), 7.78 (d, J=8.2 Hz, 1H), 7.52 (t, J=2.9 Hz, 1H), 7.45 (d, J=8.1 Hz, 1H), 7.29 (d, J=9.9 Hz, 1H), 6.95 (t, J=2.7 Hz, 1H), 6.60 (t, J=2.1 Hz, 1H), 4.91 (s, 1H), 3.01-3.03 (m, 1H), 2.95-3.00 (m, 3H), 2.85-2.88 (m, 2H), 1.56-1.70 (m, 3H), 1.30-1.51 (m, 1H), 1.06 (d, J=6.9 Hz, 3H).

### Example 7: Synthesis of Compound 112

### Synthesis of Intermediate B15

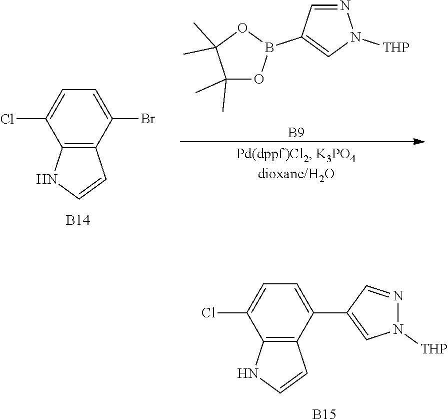

A mixture of 4-bromo-7-chloro-1H-indole (1 g, 4.34 mmol, 1 equiv), 1-(oxan-2-yl)-4-(4,4,5,5-tetramethyl-1,3,2-dioxaborolan-2-yl)pyrazole (B14; 1.33 g, 4.77 mmol, 1.1 equiv), K₃PO₄ (2.76 g, 13.02 mmol, 3 equiv) and Pd(dppf)Cl₂—CH₂Cl₂ (0.18 g, 0.22 mmol, 0.05 equiv) in dioxane (16.0 mL) and H₂O (4.0 mL) was stirred in a 40 mL sample bottle for 3h at 80° C. with deoxygenation. The resulting mixture was concentrated under reduced pressure, and the residue was purified by silica gel column chromatography, eluting with petroleum ether/ethyl acetate (3:1) to afford 7-chloro-4-[1-(oxan-2-yl)pyrazol-4-yl]-1H-indole (B15; 1.23 g) as an oil.

### Synthesis of Intermediate B16

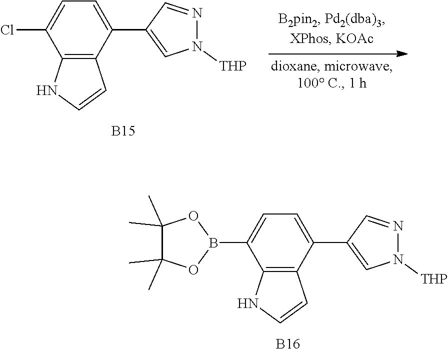

A mixture of KOAc (1.07 g, 10.94 mmol, 3 equiv), B₂Pin₂ (2.04 g, 8.02 mmol, 2.2 equiv), 7-chloro-4-[1-(oxan-2-yl)pyrazol-4-yl]-1H-indole (B15; 1.10 g, 3.65 mmol, 1 equiv), KOAc (1.07 g, 10.94 mmol, 3 equiv) and Pd₂(dba)₃-CHCl₃ (0.38 g, 0.37 mmol, 0.1 equiv) in dioxane (40.0 mL) was stirred for 1h at 100° C. with deoxygenation, in a microwave reactor. The reaction was quenched with H₂O at 0° C., and the aqueous layer was extracted with ethyl acetate (100 mL×3). The resulting mixture was washed with saturated aqueous NaCl, and dried using Na₂SO₂, and solids were removed by filtration. The filtrate was concentrated under reduced pressure to provide 4-[1-(oxan-2-yl)pyrazol-4-yl]-7-(4,4,5,5-tetramethyl-1,3,2-dioxaborolan-2-yl)-1H-indole (B16; 1.4 g) as an oil. LCMS (ES, m/z): 394 [M+H]⁺.

### Synthesis of Intermediate B22

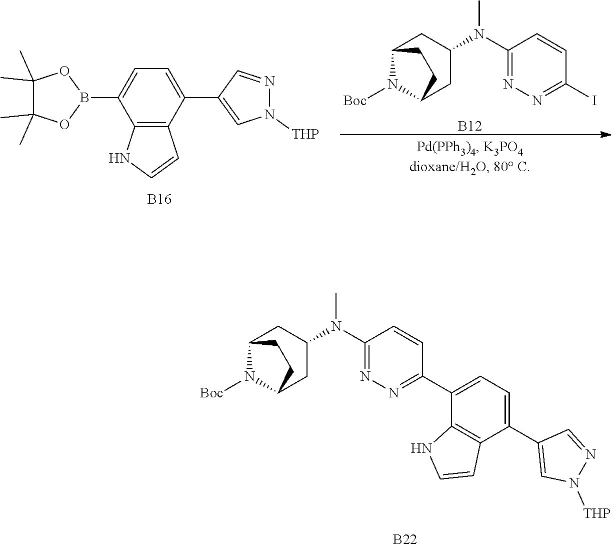

A mixture of 4-[1-(oxan-2-yl)pyrazol-4-yl]-7-(4,4,5,5-tetramethyl-1,3,2-dioxaborolan-2-yl)-1H-indole (B16; 1.23 g, 3.13 mmol, 1 equiv), tert-butyl (1R,3S,5S)-3-[(6-iodopyridazin-3-yl)(methyl)amino]-8-azabicyclo[3.2.1]octane-8-carboxylate (B12; 2.08 g, 4.69 mmol, 1.5 equiv), K₃PO₄ (1.99 g, 9.38 mmol, 3 equiv) and Pd(PPh₃)₄ (0.36 g, 0.31 mmol, 0.1 equiv) in dioxane (16 mL) and H₂O (4 mL) was stirred for 4h at 100° C. with deoxygenation, and the reaction was quenched with H₂O at 0° C. The aqueous layer was extracted with ethyl acetate (100 mL×3), and the resulting mixture was washed with saturated aqueous NaCl (100 mL), and dried with Na₂SO₄. The resulting mixture was filtered, and the filter cake was washed with ethyl acetate (50 mL×3). The filtrate was concentrated under reduced pressure, and the residue was purified by silica gel column chromatography, eluting with petroleum ether/ethyl acetate (1:1) to afford tert-butyl (1R,3S,5S)-3-[methyl(6-[4-[1-(oxan-2-yl)pyrazol-4-yl]-1H-indol-7-yl]pyridazin-3-yl)amino]-8-azabicyclo[3.2.1]octane-8-carboxylate (B22; 800 mg) as a solid. LCMS (ES, m/z): 584[M+H]⁺.

### Synthesis of Compound 112

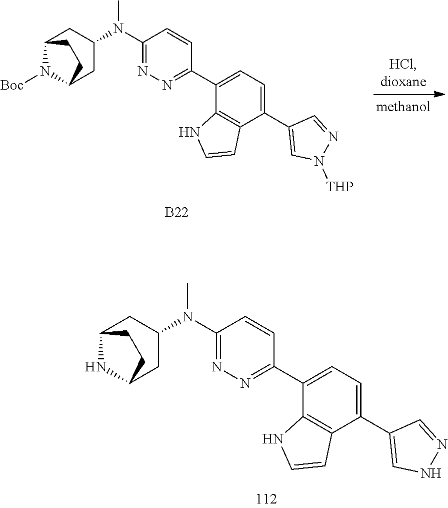

A mixture of tert-butyl (1R,3S,5S)-3-[methyl(6-[4-[1-(oxan-2-yl)pyrazol-4-yl]-1H-indol-7-yl]pyridazin-3-yl)amino]-8-azabicyclo[3.2.1]octane-8-carboxylate (B22; 800 mg), HCl in 1,4-dioxane (8.0 mL), dioxane (8.0 mL), and MeOH (3.0 mL) was stirred for 2h at 0° C. under a nitrogen atmosphere in a purged 50 mL 3-necked round-bottom flask. The mixture was filtered, and the filtrate was concentrated under reduced pressure. The crude product was purified by preparative HPLC to afford (1R,3S,5S)—N-methyl-N-[6-[4-(1H-pyrazol-4-yl)-1H-indol-7-yl]pyridazin-3-yl]-8-azabicyclo[3.2.1] octan-3-amine (Compound 112; 8 mg) as a solid. LCMS (ES, m/z): 400 [M+H]⁺. ¹H NMR (400 MHz, DMSO-d₆) δ 11.55 (s, 1H), 8.15 (d, J=16.9 Hz, 3H), 7.67 (d, J=7.4 Hz, 1H), 7.49 (s, 1H), 7.34 (d, J=7.7 Hz, 1H), 7.24 (d, J=9.3 Hz, 1H), 6.83 (s, 1H), 5.05 (s, 1H), 3.55 (s, 1H), 2.96 (s, 3H), 1.85 (s, 2H), 1.78 (s, 4H), 1.59 (s, 2H).

### Example 8: Synthesis of Compound 113

### Synthesis of Intermediate B23

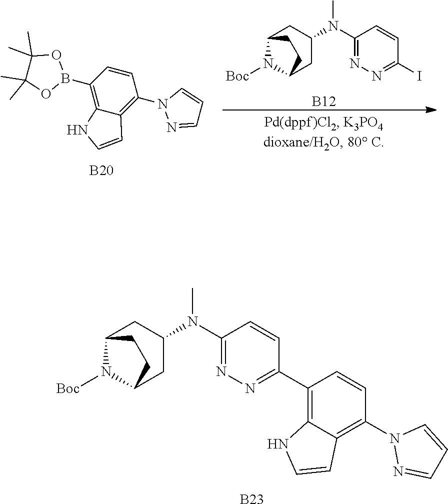

A 20-mL round-bottom flask was purged and maintained under an atmosphere of nitrogen, and 4-(pyrazol-1-yl)-7-(4,4,5,5-tetramethyl-1,3,2-dioxaborolan-2-yl)-1H-indole (B20; 500 mg, 1.61 mmol, 1.0 equiv), tert-butyl (1R,3S,5S)-3-[(6-iodopyridazin-3-yl)(methyl)amino]-8-azabicyclo[3.2.1]octane-8-carboxylate (B12; 1077 mg, 2.43 mmol, 1.5 equiv), Pd(dppf)Cl₂ (59 mg, 0.081 mmol, 0.05 equiv), K₃PO₄ (1029 mg, 4.85 mmol, 3.0 equiv), 1,4-dioxane (4 mL), and H₂O (1 mL) were added to the flask. The resulting solution was stirred for 2h at 80° C., and the reaction was quenched by the addition of water/ice (10 mL). The resulting solution was extracted with ethyl acetate (3×10 mL) and the combined organic layers were dried over anhydrous sodium sulfate. The residue was purified by silica gel column chromatography eluting with ethyl acetate/petroleum ether (1:1), to provide tert-butyl (1R,3S,5S)-3-[methyl([6-[4-(pyrazol-1-yl)-1H-indol-7-yl]pyridazin-3-yl])amino]-8-azabicyclo[3.2.1]octane-8-carboxylate (B23; 200 mg) as a solid. LCMS (ES, m/z): 500 [M+H]⁺.

### Synthesis of Compound 113

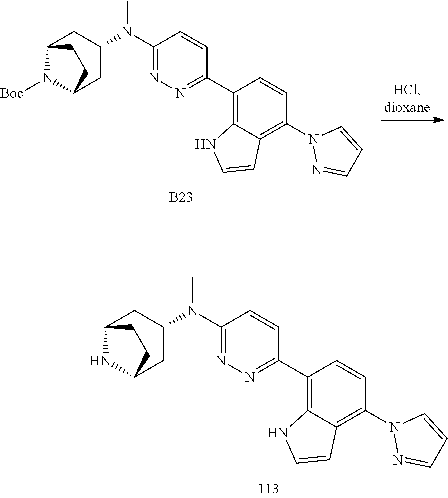

A 10-mL round-bottom flask was purged and maintained under an atmosphere of nitrogen, and tert-butyl (1R,3R,5S)-3-[methyl([6-[4-(pyrazol-1-yl)-1H-indol-7-yl]pyridazin-3-yl])amino]-8-azabicyclo[3.2.1]octane-8-carboxylate (B23; 200 mg), and 4M HCl in 1,4-dioxane (2 mL) were added to the flask. The resulting solution was stirred for 1h at 25° C., then concentrated. The crude product was purified by preparative HPLC (Condition 4, Gradient 1) to provide (1R,3R,5S)—N-methyl-N-[6-[4-(pyrazol-1-yl)-1H-indol-7-yl]pyridazin-3-yl]-8-azabicyclo[3.2.1]octan-3-amine (Compound 113; 19 mg) as a solid. LCMS (ES, m/z): 400 [M+H]⁺. 1H NMR (400 MHz, Methanol-d₄) δ 8.27 (d, J=2.4 Hz, 1H), 8.14 (dd, J=9.2, 5.4 Hz, 1H), 7.83 (dd, J=1.9, 0.6 Hz, 1H), 7.76 (dd, J=8.1, 3.5 Hz, 1H), 7.53 (dd, J=3.3, 1.1 Hz, 1H), 7.41 (dd, J=8.0, 1.8 Hz, 1H), 7.27 (dd, J=9.7, 6.1 Hz, 1H), 6.84 (d, J=3.3 Hz, 1H), 6.60-6.66 (m, 1H), 5.20-5.23 (m, 1H), 3.72 (s, 2H), 3.01-3.03 (m, 3H), 1.98-2.02 (m, 6H), 1.74-1.78 (m, 2H).

### Example 9: Synthesis of Compound 115

### Synthesis of Intermediate B25

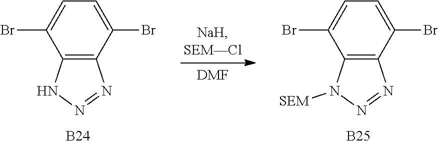

A 250-mL 3-necked round-bottom flask was purged and maintained under an atmosphere of nitrogen, and 4,7-dibromo-1H-1,2,3-benzotriazole (B24; 2 g, 7.222 mmol, 1 equiv) and DMF (30 mL) were added to the flask. The resulting solution was cooled to 0° C. in an ice/salt bath, and sodium hydride was slowly added (346.6 mg, 14.445 mmol, 2 equiv), then the mixture was warmed to 25° C., and stirred for 0.5h. SEM-Cl (1.32 g, 7.945 mmol, 1.10 equiv) was then added to the mixture dropwise, and the mixture was stirred for an additional 3h. The reaction was then quenched by pouring onto water/ice (150 mL). The resulting solution was extracted with ethyl acetate (3×50 mL), and the combined organic layers were dried with anhydrous sodium sulfate, and purified by silica gel column chromatography eluting with ethyl acetate/petroleum ether (1:4), to provide 4,7-dibromo-1-[[2-(trimethylsilyl)ethoxy]methyl]-1,2,3-benzotriazole (B25; 1.82 g) as an oil. LCMS (ES, m/z): 408 [M+H]⁺. ¹H NMR (400 MHz, Chloroform-d) δ 7.58 (d, J=8.0 Hz, 1H), 7.47 (d, J=8.0 Hz, 1H), 6.26 (s, 2H), 3.69-3.61 (m, 2H), 0.98-0.89 (m, 2H).

### Synthesis of Intermediate B26

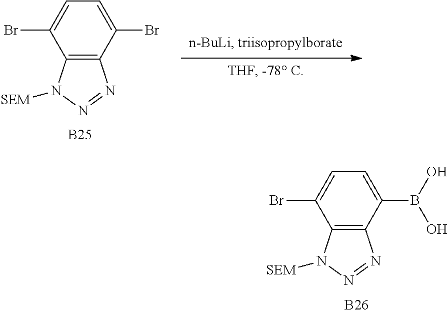

A 100-mL 3-necked round-bottom flask was purged and maintained under an atmosphere of nitrogen, and 4,7-dibromo-1-[[2-(trimethylsilyl)ethoxy]methyl]-1,2,3-benzotriazole (B25; 863.0 mg, 2.119 mmol, 1 equiv) and tetrahydrofuran (20 mL) were added to the flask, and the resulting solution was cooled to −78° C. n-BuLi in hexanes (142.6 mg, 2.225 mmol, 1.05 equiv) was then slowly added to the mixture, which was stirred for 1h at −78° C. Next, triisopropyl borate (797.2 mg, 4.239 mmol, 2 equiv) was added dropwise, and the mixture was warmed to 25° C. and stirred for an additional 1h. The reaction was then quenched by the addition of ice water (10 mL). The resulting solution was extracted with ethyl acetate (3×10 mL), and the combined organic layers were dried with anhydrous sodium sulfate, then purified by C18 column flash chromatography, eluting with acetonitrile/H₂O (1:1), to provide 7-bromo-1-[[2-(trimethylsilyl)ethoxy]methyl]-1,2,3-benzotriazol-4-ylboronic acid (B26; 542 mg) as a solid. LCMS (ES, m/z): 374 [M+H]⁺.

### Synthesis of Intermediate B27

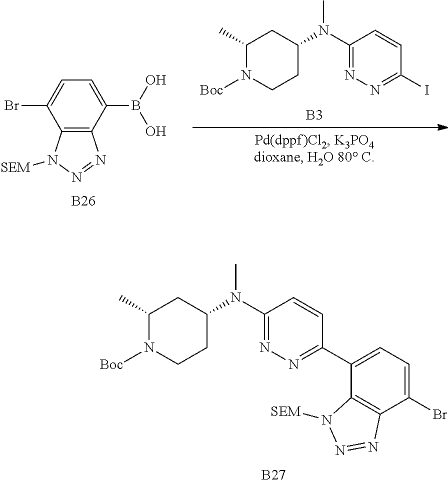

A 30 mL round-bottom flask was purged and maintained under an atmosphere of nitrogen, and 7-bromo-1-[[2-(trimethylsilyl)ethoxy]methyl]-1,2,3-benzotriazol-4-ylboronic acid (B26; 520.0 mg, 1.397 mmol, 1 equiv), tert-butyl (2R,4R)-4-[(6-iodopyridazin-3-yl)(methyl)amino]-2-methylpiperidine-1-carboxylate (B3; 543.7 mg, 1.258 mmol, 0.90 equiv), Pd(dppf)Cl₂—CH₂Cl₂ (57.1 mg, 0.070 mmol, 0.05 equiv), K₃PO₄ (889.9 mg, 4.192 mmol, 3 equiv), dioxane (10 mL), and H₂O (2 mL) were added to the flask, and the resulting solution was stirred for 12h at 80° C. The solution was then extracted with ethyl acetate (3×20 mL) and dried over anhydrous sodium sulfate. The residue was purified by silica gel column chromatography eluting with ethyl acetate/petroleum ether (1:1), to provide tert-butyl (2R,4R)-4-[[6-(7-bromo-3-[[2-(trimethylsilyl)ethoxy]methyl]-1,2,3-benzotriazol-4-yl)pyridazin-3-yl](methyl)amino]-2-methylpiperidine-1-carboxylate (B27; 397 mg) as a solid. LCMS (ES, m/z): 634 [M+H]⁺.

### Synthesis of Intermediate B28

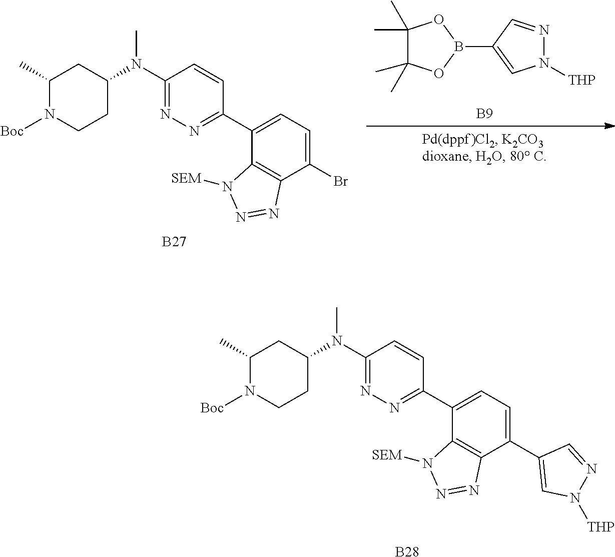

A 25-mL round-bottom flask was purged and maintained under an atmosphere of nitrogen, and tert-butyl (2R,4R)-4-[[6-(7-bromo-3-[[2-(trimethylsilyl)ethoxy]methyl]-1,2,3-benzotriazol-4-yl)pyridazin-3-yl](methyl)amino]-2-methylpiperidine-1-carboxylate (B27; 181.0 mg, 0.286 mmol, 1 equiv), 1-(oxan-2-yl)-4-(4,4,5,5-tetramethyl-1,3,2-dioxaborolan-2-yl)pyrazole (B9; 87.5 mg, 0.315 mmol, 1.10 equiv), Pd(dppf)Cl₂—CH₂Cl₂ (23.4 mg, 0.029 mmol, 0.10 equiv), K₂CO₃ (118.6 mg, 0.858 mmol, 3 equiv), dioxane (5 mL), and H₂O (1 mL) were added to the flask. The resulting solution was stirred for 4h at 80° C., and then extracted with ethyl acetate (3×10 mL) and dried over anhydrous sodium sulfate. The residue was purified by silica gel column chromatography eluting with ethyl acetate/petroleum ether (1:1), to provide tert-butyl (2R,4R)-2-methyl-4-[methyl(6-[7-[1-(oxan-2-yl)pyrazol-4-yl]-3-[[2-(trimethylsilyl)ethoxy]methyl]-1,2,3-benzotriazol-4-yl]pyridazin-3-yl)amino]piperidine-1-carboxylate (B28; 193 mg) as a solid. LCMS (ES, m/z): 704 [M+H]⁺.

### Synthesis of Compound 115

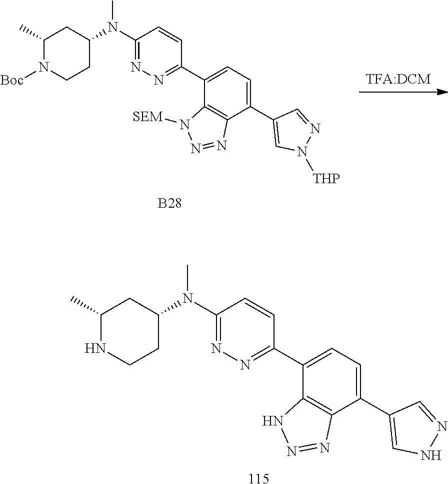

tert-Butyl (2R,4R)-2-methyl-4-[methyl(6-[7-[1-(oxan-2-yl) pyrazol-4-yl]-3-[[2-(trimethylsilyl)ethoxy]methyl]-1,2,3-benzotriazol-4-yl]pyridazin-3-yl)amino]piperidine-1-carboxylate (B28; 180.0 mg), dichloromethane (1 mL), and trifluoroacetic acid (3 mL) were added to a 10 mL round bottom flask, and the resulting solution was stirred for 4h at 25° C. The crude product was purified by preparative HPLC (Condition 4, Gradient 2), to provide N-methyl-N-[(2R,4R)-2-methylpiperidin-4-yl]-6-[7-(prop-1-en-2-yl)-3H-1,2,3-benzotriazol-4-yl]pyridazin-3-amine (Compound 115; 32.7 mg) as a solid. LCMS (ES, m/z): 390 [M+H]⁺. ¹H NMR (400 MHz, DMSO-d₆) δ 13.13 (s, 1H), 8.60-8.44 (m, 3H), 8.03 (d, J=7.7 Hz, 1H), 7.70 (d, J=7.6 Hz, 1H), 7.28 (d, J=9.8 Hz, 1H), 4.74 (s, 1H), 3.01 (s, 3H), 2.78 (ddt, J=27.0, 11.9, 5.3 Hz, 2H), 1.69 (td, J=11.5, 4.4 Hz, 3H), 1.42 (q, J=11.7 Hz, 1H), 1.09 (d, J=6.2 Hz, 3H).

### Example 10: Synthesis of Compound 117

### Synthesis of Intermediate B29

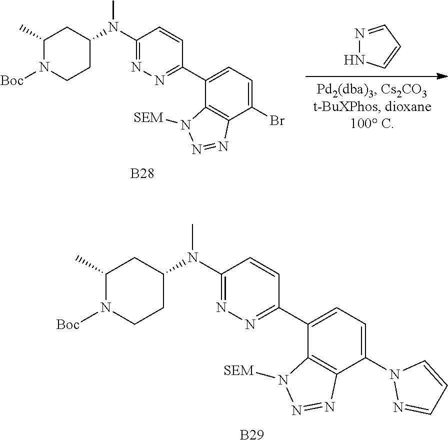

A 25-mL round-bottom flask was purged and maintained under an atmosphere of nitrogen, and tert-butyl (2R,4R)-4-[[6-(7-bromo-3-[[2-(trimethylsilyl)ethoxy]methyl]-1,2,3-benzotriazol-4-yl)pyridazin-3-yl](methyl)amino]-2-methylpiperidine-1-carboxylate (B27 from Example 10; 196.0 mg, 0.310 mmol, 1.0 equiv), pyrazole (31.6 mg, 0.465 mmol, 1.5 equiv), Pd₂(dba)₃ (28.4 mg, 0.031 mmol, 0.1 equiv), t-BuXPhos (26.3 mg, 0.062 mmol, 0.2 equiv), Cs₂CO₃ (302.8 mg, 0.929 mmol, 3.0 equiv), and dioxane (5 mL) were added to the flask. The resulting solution was stirred for 4h at 100° C., and then extracted with ethyl acetate (3×10 mL), and dried over anhydrous sodium sulfate. The residue was purified by silica gel column chromatography eluting with ethyl acetate/petroleum ether (1:1) to provide tert-butyl (2R,4R)-2-methyl-4-[methyl([6-[7-(pyrazol-1-yl)-3-[[2-(trimethylsilyl)ethoxy]methyl]-1,2,3-benzotriazol-4-yl]pyridazin-3-yl])amino]piperidine-1-carboxylate (B29; 160 mg) as a solid. LCMS (ES, m/z): 620 [M+H]⁺.

### Synthesis of Compound 117

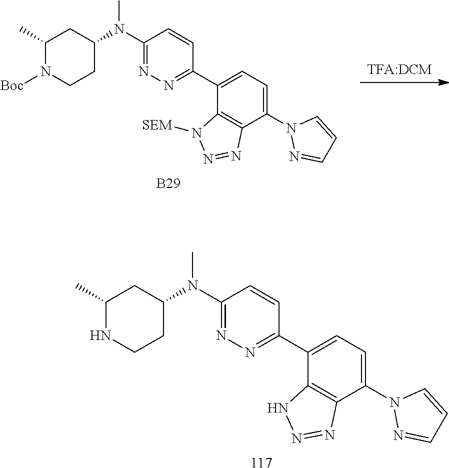

tert-Butyl (2R,4R)-2-methyl-4-[methyl([6-[7-(pyrazol-1-yl)-3-[[2-(trimethylsilyl)ethoxy]methyl]-1,2,3-benzotriazol-4-yl]pyridazin-3-yl])amino]piperidine-1-carboxylate (B29; 150.0 mg), dichloromethane (1 mL), and trifluoroacetic acid (3 mL) were placed in a 10-mL round-bottom flask, and the resulting solution was stirred for 4h at 25° C. The crude product was purified by preparative HPLC (Condition 4, Gradient 2), to provide N-methyl-N-[(2R,4R)-2-methylpiperidin-4-yl]-6-[7-(pyrazol-1-yl)-3H-1,2,3-benzotriazol-4-yl]pyridazin-3-amine (Compound 117; 22.8 mg) as a solid. LCMS (ES, m/z): 390 [M+H]⁺. ¹H NMR (400 MHz, Chloroform-d) δ 9.58 (s, 1H), δ 8.96 (d, J=1.2 Hz, 1H), 8.07 (d, J=1.2 Hz, 1H), 7.83 (d, J=2.8 Hz, 1H), 7.32-7.29 (m, 2H), 6.62 (s, 1H), 4.93-4.78 (s, 1H), 3.30-3.17 (m, 4H), 2.93 (s, 3H), 1.97-1.67 (m, 4H), 1.21 (d, J=1.2 Hz, 3H).

### Example 11: Synthesis of Compound 118

### Synthesis of Intermediate B30

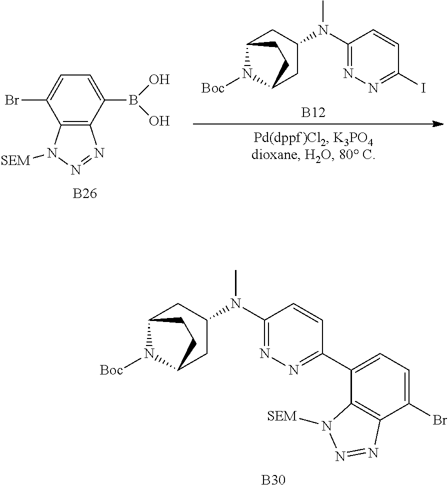

A 30-mL round-bottom flask was purged and maintained under an atmosphere of nitrogen, and 7-bromo-1-[[2-(trimethylsilyl)ethoxy]methyl]-1,2,3-benzotriazol-4-ylboronic acid (B26 from Example 10; 542.0 mg, 1.457 mmol, 1 equiv), tert-butyl (1R,3S,5S)-3-[(6-iodopyridazin-3-yl)(methyl)amino]-8-azabicyclo[3.2.1]octane-8-carboxylate (B12; 647.2 mg, 1.457 mmol, 1 equiv), Pd(dppf)Cl₂—CH₂Cl₂ (59.5 mg, 0.073 mmol, 0.05 equiv), K₃PO₄ (927.6 mg, 4.370 mmol, 3 equiv), dioxane (10 mL), and H₂O (2 mL) were added to the flask, and the resulting solution was stirred for 12h at 80° C. The solution was then extracted with ethyl acetate (3×10 mL) and dried over anhydrous sodium sulfate. The residue was purified by silica gel column chromatography eluting with ethyl acetate/petroleum ether (1:1) to provide tert-butyl (1R,3S,5S)-3-[[6-(7-bromo-3-[[2-(trimethylsilyl)ethoxy]methyl]-1,2,3-benzotriazol-4-yl)pyridazin-3-yl](methyl)amino]-8-azabicyclo[3.2.1]octane-8-carboxylate (B30; 415 mg) as a solid. LCMS (ES, m/z): 646 [M+H]⁺.

### Synthesis of Intermediate B31

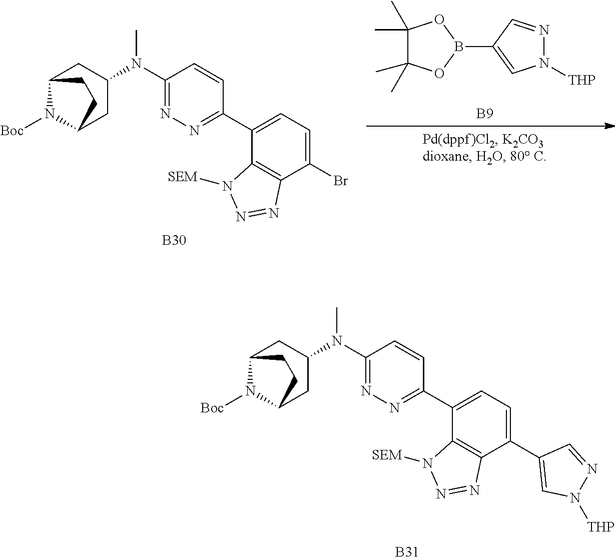

A 20-mL round-bottom flask was purged and maintained under an atmosphere of nitrogen, and tert-butyl (1R,3S,5S)-3-[[6-(7-bromo-3-[[2-(trimethylsilyl)ethoxy]methyl]-1,2,3-benzotriazol-4-yl)pyridazin-3-yl](methyl)amino]-8-azabicyclo[3.2.1]octane-8-carboxylate (B30; 20.0 mg, 0.031 mmol, 1 equiv), 1-(oxan-2-yl)-4-(4,4,5,5-tetramethyl-1,3,2-dioxaborolan-2-yl)pyrazole (B9; 8.6 mg, 0.031 mmol, 1 equiv), Pd(dppf)Cl₂—CH₂Cl₂ (2.5 mg, 0.003 mmol, 0.10 equiv), K₂CO₃ (76.0 mg, 0.093 mmol, 3 equiv), dioxane (5 mL), and H₂O (1 mL) were added to the flask, and the resulting solution was stirred for 4h at 80° C. The solution was then extracted with ethyl acetate (3×10 mL) and dried over anhydrous sodium sulfate. The residue was purified by silica gel column chromatography eluting with ethyl acetate/petroleum ether (1:1) to provide tert-butyl (1R,3S,5S)-3-[methyl(6-[7-[1-(oxan-2-yl)pyrazol-4-yl]-3-[[2-(trimethylsilyl)ethoxy]methyl]-1,2,3-benzotriazol-4-yl]pyridazin-3-yl)amino]-8-azabicyclo[3.2.1]octane-8-carboxylate (B31; 126 mg) as a solid. LCMS (ES, m/z): 716 [M+H]⁺.

### Synthesis of Compound 118

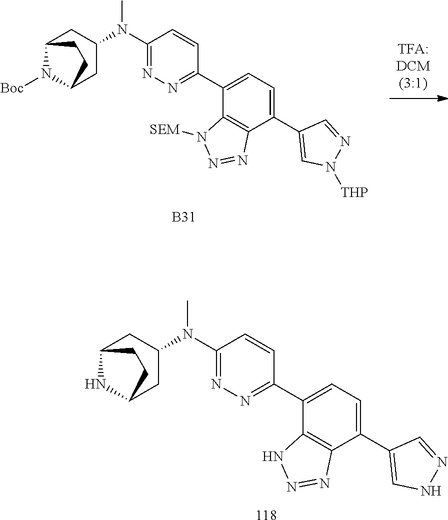

tert-Butyl (1R,3S,5S)-3-[methyl(6-[7-[1-(oxan-2-yl)pyrazol-4-yl]-3-[[2-(trimethylsilyl)ethoxy]methyl]-1,2,3-benzotriazol-4-yl]pyridazin-3-yl)amino]-8-azabicyclo[3.2.1]octane-8-carboxylate (B31; 126.0 mg), dichloromethane (3 mL), and trifluoroacetic acid (9 mL) were placed in a 25-mL round-bottom flask, and the resulting solution was stirred for 4h at 25° C. The crude product was purified by preparative HPLC (Condition 4, Gradient 2) to provide (1R,3S,5S)—N-methyl-N-[6-[7-(1H-pyrazol-4-yl)-3H-1,2,3-benzotriazol-4-yl]pyridazin-3-yl]-8-azabicyclo[3.2.1]octan-3-amine (Compound 118; 30 mg) as a solid. LCMS (ES, m/z): 402 [M+H]⁺. ¹H NMR (400 MHz, DMSO-d₆) δ 13.10 (br, 1H), 8.59 (s, 2H), 8.54 (d, J=9.7 Hz, 1H), 8.01 (d, J=7.6 Hz, 1H), 7.66 (d, J=7.6 Hz, 1H), 7.26 (d, J=9.7 Hz, 1H), 5.14 (dq, J=11.9, 5.9 Hz, 1H), 2.97 (s, 3H), 1.95 (td, J=12.4, 3.0 Hz, 2H), 1.87 (s, 4H), 1.70-1.60 (m, 2H).

### Example 12: Synthesis of Compound 119

### Synthesis of Intermediate B32

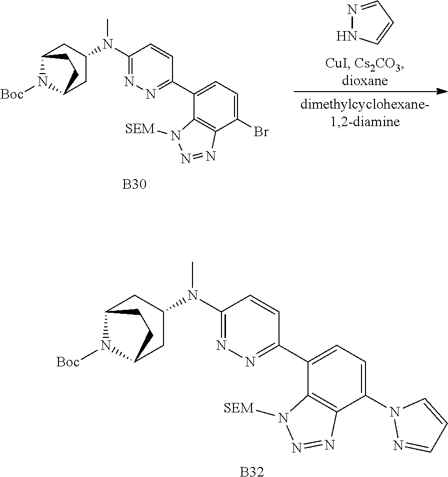

A 25-mL round-bottom flask was purged and maintained under an atmosphere of nitrogen, and tert-butyl(1R,3S,5S)-3-[[6-(7-bromo-3-[[2-(trimethylsilyl)ethoxy]methyl]-1,2,3-benzotriazol-4-yl)pyridazin-3-yl](methyl)amino]-8-azabicyclo[3.2.1]octane-8-carboxylate (B30 from Example 12; 220.0 mg, 0.341 mmol, 1 equiv), pyrazole (34.8 mg, 0.512 mmol, 1.50 equiv), CuI (6.5 mg, 0.034 mmol, 0.10 equiv), Cs₂CO₃ (333.5 mg, 1.024 mmol, 3 equiv), N1,N2-dimethylcyclohexane-1,2-diamine (4.8 mg, 0.034 mmol, 0.10 equiv), and dioxane (5 mL) were added to the flask, and the resulting solution was stirred for 12h at 100° C. The solution was then extracted with ethyl acetate (3×10 mL), and the organic phase was concentrated. The residue was purified by silica gel column chromatography eluting with ethyl acetate/petroleum ether (2:1) to provide tert-butyl (1R,3S,5S)-3-[methyl([6-[7-(pyrazol-1-yl)-3-[[2-(trimethylsilyl)ethoxy]methyl]-1,2,3-benzotriazol-4-yl]pyridazin-3-yl])amino]-8-azabicyclo[3.2.1]octane-8-carboxylate (B32; 34 mg) as a solid. LCMS (ES, m/z): 632 [M+H]⁺.

### Synthesis of Compound 119

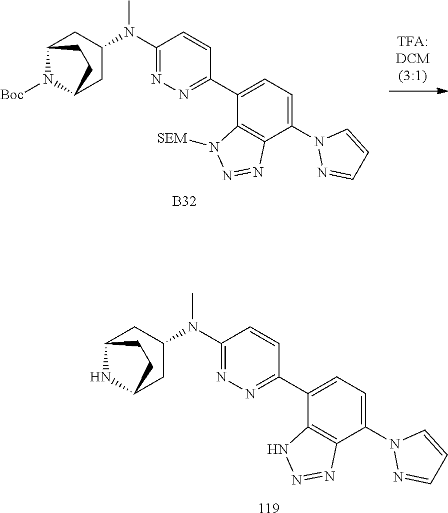

tert-Butyl (1R,3S,5S)-3-[methyl([6-[7-(pyrazol-1-yl)-3-[[2-(trimethylsilyl)ethoxy]methyl]-1,2,3-benzotriazol-4-yl]pyridazin-3-yl])amino]-8-azabicyclo[3.2.1]octane-8-carboxylate (B32; 20.0 mg), dichloromethane (1 mL), and trifluoroacetic acid (3 mL) were placed in a 10-mL round-bottom flask, and the resulting solution was stirred for 4h at 25° C. The crude product was purified by preparative HPLC (Condition 4, Gradient 2), to provide (1R,3S,5S)—N-methyl-N-[6-[7-(pyrazol-1-yl)-3H-1,2,3-benzotriazol-4-yl]pyridazin-3-yl]-8-azabicyclo[3.2.1]octan-3-amine (Compound 119; 9.3 mg) as a solid. LCMS (ES, m/z): 402 [M+H]⁺. ¹H NMR (400 MHz, DMSO-d₆) δ 9.60 (d, J=2.5 Hz, 1H), 9.05 (d, J=9.7 Hz, 1H), 8.09 (d, J=7.9 Hz, 1H), 7.85-7.75 (m, 2H), 7.30 (d, J=9.7 Hz, 1H), 6.62 (t, J=2.2 Hz, 1H), 5.22 (dt, J=12.0, 6.1 Hz, 1H), 4.06 (s, 2H), 2.99 (s, 3H), 2.17-2.04 (m, 6H), 2.02 (s, 1H), 1.84-1.74 (m, 2H).

### Example 13: Synthesis of Compound 121

### Synthesis of Intermediate B34

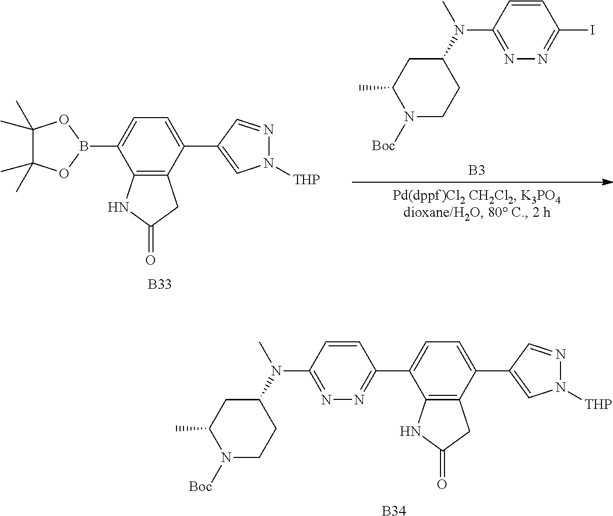

4-[1-(Oxan-2-yl)pyrazol-4-yl]-7-(4,4,5,5-tetramethyl-1,3,2-dioxaborolan-2-yl)-1,3-dihydroindol-2-one (B33; 150.0 mg, 0.37 mmol, 1 equiv), tert-butyl (2R,4R)-4-[(6-iodopyridazin-3-yl)(methyl)amino]-2-methylpiperidine-1-carboxylate (B3; 158.44 mg, 0.37 mmol, 1 equiv), Pd(dppf)Cl₂—CH₂Cl₂ (29.93 mg, 0.04 mmol, 0.10 equiv), K₃PO₄ (233.38 mg, 1.10 mmol, 3 equiv), dioxane (15 mL), and water (3 mL) were added to a 40 mL vial, and the resulting mixture was stirred for 2h at 80° C. under a nitrogen atmosphere. The reaction was quenched with water, and the resulting mixture was extracted with ethyl acetate (2×10 mL). The combined organic layers were washed with brine (1×10 mL) and dried over anhydrous Na₂SO₄. After filtration, the filtrate was concentrated under reduced pressure. The residue was purified by preparative TLC, eluting with dichloromethane/methanol (20:1) to afford tert-butyl (2R,4R)-2-methyl-4-[methyl(6-[4-[1-(oxan-2-yl)pyrazol-4-yl]-2-oxo-1,3-dihydroindol-7-yl]pyridazin-3-yl)amino]piperidine-1-carboxylate (B34; 14.0 mg) as a solid. LCMS (ES, m/z): 588 [M+H]⁺.

### Synthesis of Compound 121

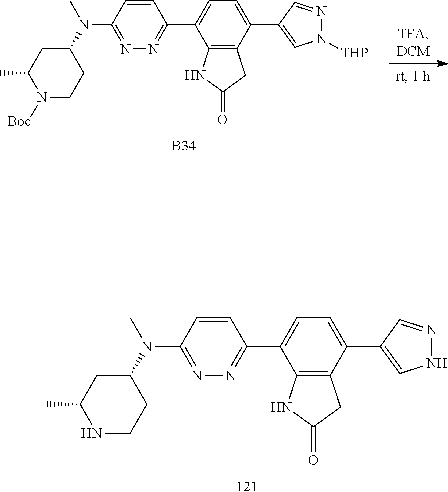

tert-Butyl (2R,4R)-2-methyl-4-[methyl(6-[4-[1-(oxan-2-yl)pyrazol-4-yl]-2-oxo-1,3-dihydro indol-7-yl]pyridazin-3-yl)amino]piperidine-1-carboxylate (B34; 14.0 mg, 0.02 mmol, 1 equiv), trifluoroacetic acid (0.4 mL) and dichloromethane (2.0 mL) were added to a 4 mL vial, and the resulting mixture was stirred for 1h at room temperature. The resulting mixture was concentrated under reduced pressure, and the crude product was purified by preparative HPLC (Condition 1), to afford 7-(6-[methyl[(2R,4R)-2-methylpiperidin-4-yl]amino]pyridazin-3-yl)-4-(1H-pyrazol-4-yl)-1,3-dihydroindol-2-one (Compound 121; 5.8 mg) as a solid. LC-MS (ES, m z): 404 [M+H]⁺. 1H NMR (400 MHz, Methanol-d4): δ 8.50 (d, J=10.1 Hz, 1H), 8.16 (d, J=8.6 Hz, 3H), 7.84 (d, J=8.5 Hz, 1H), 7.52 (d, J=8.4 Hz, 1H), 4.71-4.59 (m, 1H), 3.64 (dt, J=13.1, 3.4 Hz, 1H), 3.56-3.46 (m, 1H), 3.34 (s, 2H), 3.27 (s, 4H), 2.24-2.11 (m, 3H), 2.01 (q, J=12.2 Hz, 1H), 1.45 (d, J=6.4 Hz, 3H).

### Example 14: Synthesis of Compound 123

### Synthesis of Intermediate B33

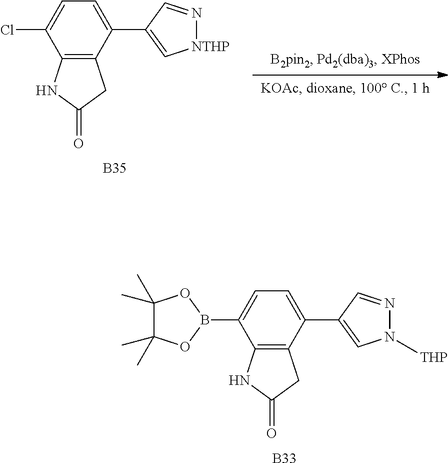

7-Chloro-4-[1-(oxan-2-yl)pyrazol-4-yl]-1,3-dihydroindol-2-one (B35; 150.0 mg, 0.47 mmol, 1 equiv), bis(pinacolato)diboron (239.74 mg, 0.94 mmol, 2 equiv), Pd₂(dba)₃ (43.23 mg, 0.05 mmol, 0.10 equiv), XPhos (45.01 mg, 0.10 mmol, 0.20 equiv), KOAc (138.98 mg, 1.42 mmol, 3 equiv), and dioxane (15 mL) were added to a 40 mL vial, and the resulting mixture was stirred for 1h at 100° C. under a nitrogen atmosphere. The crude product (B33) was used in the next step directly without further purification. LCMS (ES, m/z): 410 [M+H]⁺

### Synthesis of Intermediate B36

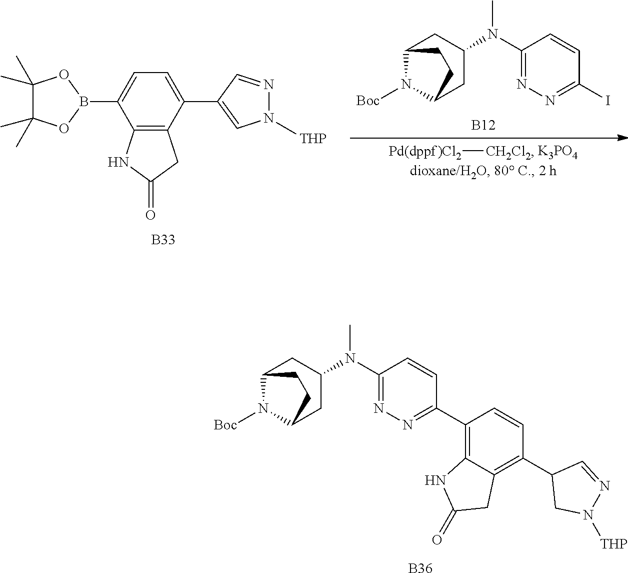

4-[1-(Oxan-2-yl)pyrazol-4-yl]-7-(4,4,5,5-tetramethyl-1,3,2-dioxaborolan-2-yl)-1,3-dihydroindol-2-one (B33; 150.0 mg, 0.366 mmol, 1 equiv), tert-butyl (1R,3S,5S)-3-[(6-iodopyridazin-3-yl)(methyl)amino]-8-azabicyclo[3.2.1]octane-8-carboxylate (B12; 162.84 mg, 0.37 mmol, 1 equiv), Pd(dppf)Cl₂—CH₂Cl₂ (29.93 mg, 0.04 mmol, 0.10 equiv), K₃PO₄ (233.38 mg, 1.10 mmol, 3 equiv), dioxane (15 mL), and water (3 mL) were added to a 40 mL vial, and the resulting mixture was stirred for 2h at 80° C. under a nitrogen atmosphere. The reaction was quenched with water, and the resulting mixture was extracted with ethyl acetate (2×10 mL). The combined organic layers were washed with brine (1×10 mL), dried over anhydrous Na₂SO₄, filtered, and concentrated under reduced pressure. The residue was purified by preparative TLC, eluting with dichloromethane/methanol (20:1) to afford tert-butyl (1R,3S,5S)-3-[methyl(6-[4-[1-(oxan-2-yl)pyrazol-4-yl]-2-oxo-1,3-dihydroindol-7-yl]pyridazin-3-yl)amino]-8-azabicyclo[3.2.1]octane-8-carboxylate (B36; 20.0 mg) as a solid. LCMS (ES, m/z): 600 [M+H]⁺.

### Synthesis of Compound 123

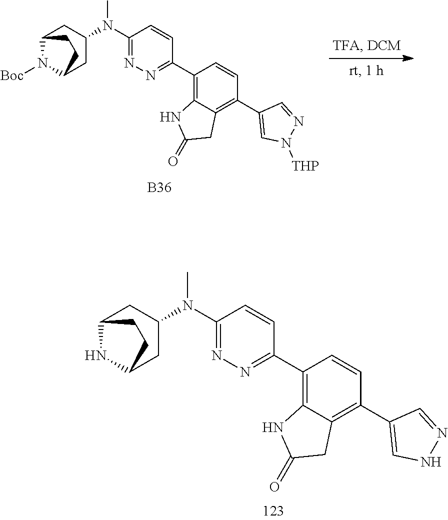

tert-Butyl (1R,3S,5S)-3-[methyl(6-[4-[1-(oxan-2-yl)pyrazol-4-yl]-2-oxo-1,3-dihydroindol-7-yl]pyridazin-3-yl)amino]-8-azabicyclo[3.2.1]octane-8-carboxylate (B36; 20.0 mg, 0.03 mmol, 1 equiv), trifluoroacetic acid (0.4 mL) and dichloromethane (2.0 mL) were added to a 4 mL vial, and the resulting mixture was stirred for 1h at room temperature, and then concentrated under reduced pressure. The crude product was purified by preparative HPLC (Condition 1), to afford 7-[6-[(1R,3S,5S)-8-azabicyclo[3.2.1]octan-3-yl(methyl)amino]pyridazin-3-yl]-4-(1H-pyrazol-4-yl)-1,3-dihydroindol-2-one (Compound 123; 1.70 mg) as a solid. LC-MS (ES, m/z): 416 [M+H]⁺. ¹H NMR (400 MHz, Methanol-d4) δ 8.49 (d, J=10.1 Hz, 1H), 8.18 (d, J=9.0 Hz, 2H), 7.83 (d, J=8.5 Hz, 1H), 7.54 (d, J=8.5 Hz, 1H), 4.77 (d, J=5.6 Hz, 1H), 4.27 (s, 2H), 3.34 (s, 2H), 3.26 (s, 3H), 2.43-2.22 (m, 6H), 2.10 (d, J=13.4 Hz, 2H).

### Example 23: Synthesis of Compound 131

### Synthesis of Intermediate B50

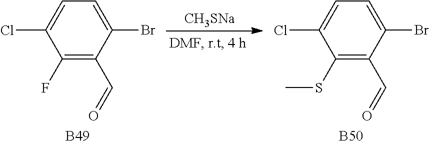

(Methylsulfanyl)sodium (1.48 g, 21.1 mmol, 1 equiv) was slowly added to a solution of 6-bromo-3-chloro-2-fluorobenzaldehyde (B49; 50 g, 21.1 mmol, 1 equiv) in dimethylformamide, and the mixture was stirred at room temperature for 4 h. The mixture was then diluted with ethyl acetate (150 mL) and water (100 mL), and the aqueous layer was extracted with ethyl acetate (2×100 mL). The combined organic layers were washed with saturated brine (200 mL), dried over anhydrous sodium sulfate, and concentrated. The residue was purified by silica gel column chromatography (120 g silica gel) eluting with 18% ethyl acetate in petroleum ether, to afford 6-bromo-3-chloro-2-(methylsulfanyl) benzaldehyde (B50; 3 g) as a solid. ¹H NMR (400 MHz, DMSO-d₆, ppm) δ 10.26 (s, 1H), 7.77 (d, J=8.7 Hz, 1H), 7.68 (d, J=8.4 Hz, 1H), 2.41 (s, 3H).

### Synthesis of Intermediate B52

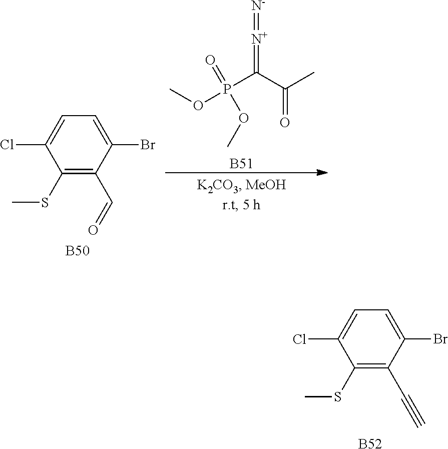

6-Bromo-3-chloro-2-(methylsulfanyl)benzaldehyde (B50; 3 g, 11.3 mmol, 1 equiv) was added to a mixture of Ohira-Bestmann reagent (B51; 3.26 g, 16.9 mmol, 1.5 equiv.) and K₂CO₃ (4.68 g, 33.9 mmol, 3 equiv) in methanol (30 mL) at 0° C. under an atmosphere of nitrogen, and the resulting mixture was warmed to room temperature and stirred for 2 h. The mixture was then diluted with diethyl ether (30 mL) and water (30 mL), and the aqueous layer was extracted with diethyl ether (2×30 mL). The combined organic layers were washed with saturated brine (50 mL), dried over anhydrous sodium sulfate and concentrated to afford a residue. The residue was purified by silica gel column chromatography (80 g silica gel column) eluting with 15% ethyl acetate in petroleum ether, to afford 1-bromo-4-chloro-2-ethynyl-3-(methylsulfanyl)benzene (B52; 2.2 g) as a solid. ¹H NMR (400 MHz, DMSO-d₆, ppm) δ 7.72 (d, J=8.7 Hz, 1H), 7.52 (d, J=8.7 Hz, 1H), 5.03 (s, 1H), 2.50 (s, 3H).

### Synthesis of Intermediate B53

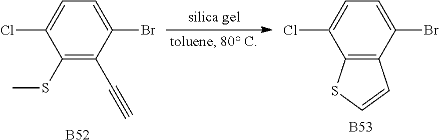

Silica gel (14 g, 233 mmol, 27.7 equiv) was added to a solution of 1-bromo-4-chloro-2-ethynyl-3-(methylsulfanyl)benzene (B52; 2.2 g, 8.4 mmol, 1 equiv) in toluene (40 mL), and the reaction mixture was stirred at 90° C. for 16 h under an atmosphere of nitrogen. The resulting mixture was then filtered, and the filter cake was washed with dichloromethane. The filtrate was then concentrated under reduced pressure to afford 4-bromo-7-chloro-1-benzothiophene (B53; 1.8 g) as a solid. ¹H NMR (400 MHz, DMSO-d₆, ppm) δ 8.07 (d, J=5.5 Hz, 1H), 7.69 (d, J=8.1 Hz, 1H), 7.54 (d, J=5.6 Hz, 1H), 7.46 (d, J=8.1 Hz, 1H).

### Synthesis of Intermediate B54

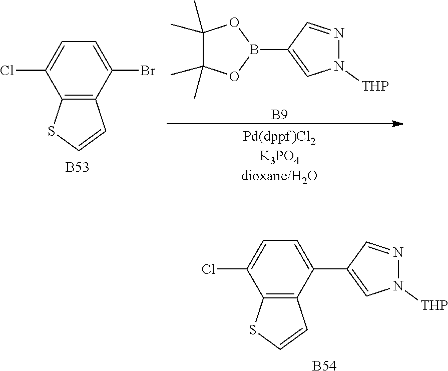

Potassium triphosphate (2.56 g, 12 mmol, 3 equiv) and Pd(dppf)Cl₂ (147.2 mg, 0.2 mmol, 0.05 equiv) were added to a solution of 4-bromo-7-chloro-1,3-benzothiazole (B53; 1 g, 4 mmol, 1 equiv) and 1-(oxan-2-yl)-4-(4,4,5,5-tetramethyl-1,3,2-dioxaborolan-2-yl)pyrazole (B9; 1.34 g, 5 mmol, 1.2 equiv) in dioxane (8 mL) and water (2 mL), and the resulting mixture was stirred overnight at 100° C. under an atmosphere of nitrogen. The mixture was then concentrated under reduced pressure and diluted with ethyl acetate (25 mL) and water (30 mL). The aqueous layer was extracted with ethyl acetate (2×25 mL), and the combined organic layers were washed with saturated brine (40 mL), dried over anhydrous sodium sulfate, and concentrated to afford a residue. The residue was purified by silica gel column chromatography (40 g silica gel), eluting with 35% ethyl acetate in petroleum ether, to afford 7-chloro-4-[1-(oxan-2-yl)pyrazol-4-yl]-1,3-benzothiazole (B54; 1 g) as an oil. LCMS (ES, m/z): 319.00 [M+H]⁺. ¹H NMR (400 MHz, DMSO-d₆, ppm) δ 8.41 (d, J=0.8 Hz, 1H), 7.98 (d, J=0.8 Hz, 1H), 7.96 (d, J=5.5 Hz, 1H), 7.77 (d, J=5.6 Hz, 1H), 7.57-7.50 (m, 2H), 5.49 (dd, J=10.1, 2.1 Hz, 1H), 4.01-3.94 (m, 1H), 3.71-3.61 (m, 1H), 2.21 (dddd, J=13.5, 10.0, 8.0, 3.3 Hz, 1H), 2.03-1.93 (m, 2H), 1.71 (qdt, J=9.2, 6.7, 3.5 Hz, 1H), 1.57 (tq, J=6.0, 3.8 Hz, 2H).

### Synthesis of Intermediate B55

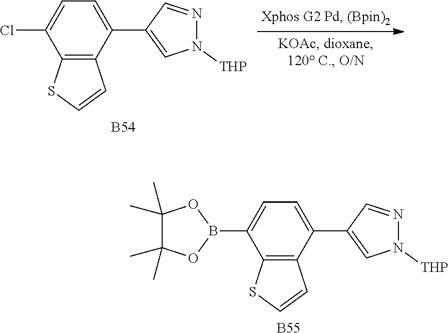

Potassium acetate (739 mg, 7.5 mmol, 3 equiv) and XPhos Pd G2 (98.6 mg, 0.13 mmol, 0.05 equiv) were added to a solution of 4-(7-chloro-1-benzothiophen-4-yl)-1-(oxan-2-yl)pyrazole (B54; 800 mg, 2.5 mmol, 1 equiv) and bis(pinacolato)diboron (765 mg, 3 mmol, 1.2 equiv) in dioxane (8 mL), and the resulting mixture was stirred overnight at 100° C. under an atmosphere of nitrogen. The mixture was then concentrated under reduced pressure and diluted with ethyl acetate (10 mL) and water (15 mL), and the aqueous layer was extracted with ethyl acetate (2×10 mL). The combined organic layers were then washed with saturated brine (20 mL), dried over anhydrous sodium sulfate, and concentrated to provide 1-(oxan-2-yl)-4-[7-(4,4,5,5-tetramethyl-1,3,2-dioxaborolan-2-yl)-1-benzothiophen-4-yl]pyrazole (B55; 1.5 g) as an oil. LCMS (ES, m/z): 411.10 [M+H]⁺.

### Synthesis of Intermediate B57

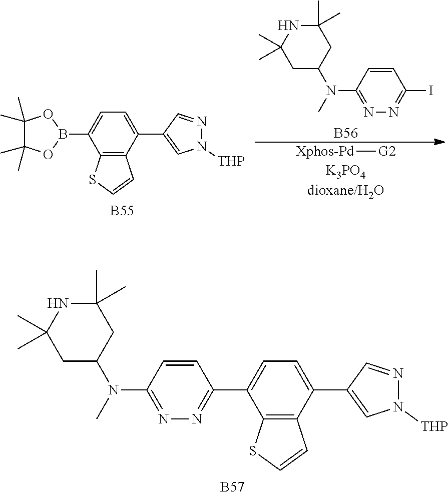

Tripotassium phosphate (510 mg, 2.4 mmol, 3 equiv) and XPhos Pd G2 (31.5 mg, 0.04 mmol, 0.05 equiv) were added to a solution of 6-iodo-N-methyl-N-(2,2,6,6-tetramethylpiperidin-4-yl)pyridazin-3-amine (B56 from Example 23; 300 mg, 0.8 mmol, 1 equiv) and 1-(oxan-2-yl)-4-[7-(4,4,5,5-tetramethyl-1,3,2-dioxaborolan-2-yl)-1-benzothiophen-4-yl]pyrazole (B55; 493 mg, 1.2 mmol, 1.5 equiv.) in dioxane (4 mL) and water (1 mL), and the resulting mixture was stirred overnight at 100° C. under an atmosphere of nitrogen. The mixture was then concentrated under reduced pressure and diluted with ethyl acetate (15 mL) and water (12 mL), and the aqueous layer was extracted with ethyl acetate (2×15 mL). The combined organic layers were washed with saturated brine (30 mL), dried over anhydrous sodium sulfate, and concentrated to provide a residue. The residue was then purified by silica gel column chromatography (40 g silica gel), eluting with 46% methanol in dichloromethane, to afford N-methyl-6-[4-[1-(oxan-2-yl)pyrazol-4-yl]-1-benzothiophen-7-yl]-N-(2,2,6,6-tetramethylpiperidin-4-yl)pyridazin-3-amine (B57; 90 mg) as a solid. LCMS (ES, m/z): 531.25 [M+H]⁺.

### Synthesis of Compound 131

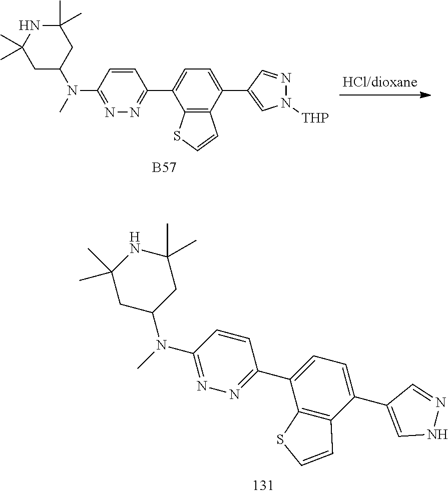

A solution of HCl in 1,4-dioxane (2 mL) was added to a solution of N-methyl-6-[4-[1-(oxan-2-yl)pyrazol-4-yl]-1-benzothiophen-7-yl]-N-(2,2,6,6-tetramethylpiperidin-4-yl)pyridazin-3-amine (B57; 90 mg, 1 equiv) in dioxane (2 mL), and the resulting mixture was stirred at room temperature for 2 h. The mixture was then concentrated under vacuum, and purified by preparative HPLC (Condition 3) to afford N-methyl-6-[4-(1H-pyrazol-4-yl)-1-benzothiophen-7-yl]-N-(2,2,6,6-tetramethylpiperidin-4-yl)pyridazin-3-amine (Compound 131; 17.8 mg) as a solid. LCMS (ES, m/z): 447.15 [M+H]⁺. ¹H NMR (400 MHz, methanol-d₄, ppm) δ 8.07 (d, J=9.8 Hz, 1H), 8.05 (s, 2H), 7.84 (d, J=7.8 Hz, 1H), 7.78-7.69 (m, 2H), 7.59 (d, J=7.7 Hz, 1H), 7.25 (d, J=9.8 Hz, 1H), 5.19 (s, 1H), 3.05 (s, 3H), 1.75 (dd, J=12.7, 3.6 Hz, 2H), 1.65 (t, J=12.5 Hz, 2H), 1.45 (s, 6H), 1.28 (s, 6H).

### Example 24: Synthesis of Compound 137

### Synthesis of Intermediate B56

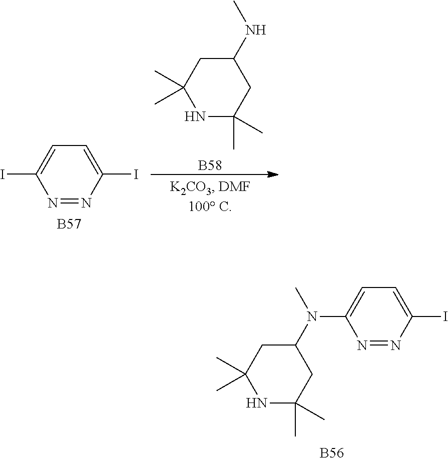

A mixture of 3,6-diiodopyridazine (3.9 g, 11.8 mmol, 1 equiv), N,2,2,6,6-pentamethylpiperidin-4-amine (2 g, 11.8 mmol, 1 equiv) and K₂CO₃ (1.6 g, 11.8 mmol, 1 equiv) in dimethylformamide (20 mL) was stirred overnight at 100° C. under an atmosphere of nitrogen. The reaction was quenched with H₂O at 0° C., and the aqueous layer was extracted with ethyl acetate (50 mL×2). The residue was purified by silica gel column chromatography, eluting with petroleum ether: ethyl acetate (0.5) to afford 6-iodo-N-methyl-N-(2,2,6,6-tetramethylpiperidin-4-yl)pyridazin-3-amine (B56; 2 g) as a solid. LCMS (ES, m/z): 375[M+H]⁺.

### Synthesis of Intermediate B59

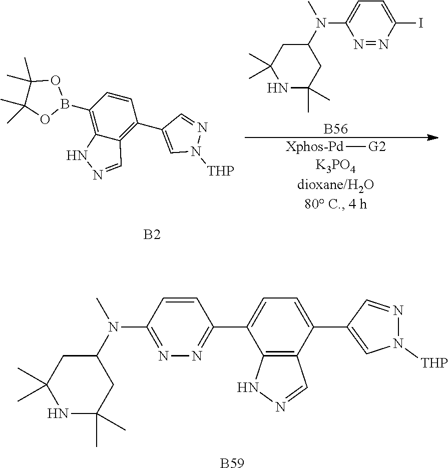

A mixture of K₃PO₄ (242.3 mg, 1.14 mmol, 3 equiv), 4-[1-(oxan-2-yl)pyrazol-4-yl]-7-(4,4,5,5-tetramethyl-1,3,2-dioxaborolan-2-yl)-1H-indazole (B2 from Example 1; 150 mg, 0.38 mmol, 1 equiv), 6-iodo-N-methyl-N-(2,2,6,6-tetramethylpiperidin-4-yl)pyridazin-3-amine (B56; 170.9 mg, 0.46 mmol, 1.2 equiv) and XPhos palladium(II) biphenyl-2-amine chloride (29.9 mg, 0.04 mmol, 0.1 equiv) in dioxane (4 mL) and H₂O (1 mL) were stirred for 4 h at 80° C. under an atmosphere of nitrogen. The reaction was quenched with H₂O at 0° C., and the aqueous layer was extracted with ethyl acetate (20 mL×2). The resulting mixture was washed with saturated sodium chloride solution (10 mL), and the organic extract was dried over sodium sulfate, filtered, and the filter cake was washed with ethyl acetate. The filtrate was concentrated under reduced pressure to provide a residue. The residue was purified by silica gel column chromatography, eluting with dichloromethane/methanol (15%) to afford N-methyl-6-[4-[1-(oxan-2-yl)pyrazol-4-yl]-1H-indazol-7-yl]-N-(2,2,6,6-tetramethylpiperidin-4-yl)pyridazin-3-amine (B59; 100 mg) as an oil. LCMS (ES, m/z): 515 [M+H]⁺.

### Synthesis of Compound 137

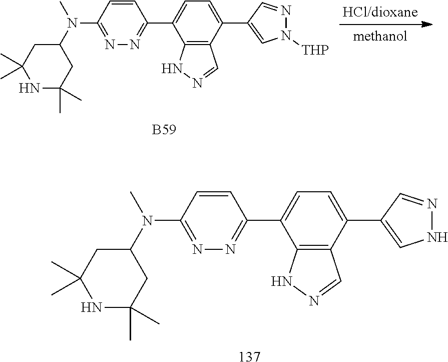

A mixture of N-methyl-6-[4-[1-(oxan-2-yl)pyrazol-4-yl]-1H-indazol-7-yl]-N-(2,2,6,6-tetramethylpiperidin-4-yl)pyridazin-3-amine (B59; 100 mg), HCl in 1,4-dioxane (2.5 mL) and MeOH (2.0 mL) was stirred for 1h at 0° C. under an atmosphere of nitrogen. The resulting crude product was purified by preparative HPLC (Condition 5), to afford N-methyl-6-[4-(1H-pyrazol-4-yl)-1H-indazol-7-yl]-N-(2,2,6,6-tetramethylpiperidin-4-yl)pyridazin-3-amine (8.5 mg) as a solid. LCMS (ES, m/z): 431[M+H]⁺. ¹H NMR (400 MHz, DMSO-d₆, ppm) δ 13.19 (s, 1H), 13.08 (s, 1H), 8.52 (s, 1H), 8.31 (s, 2H), 8.17 (d, J=9.8 Hz, 1H), 7.90 (d, J=7.7 Hz, 1H), 7.48 (d, J=7.6 Hz, 1H), 7.26 (d, J=9.8 Hz, 1H), 5.08 (s, 1H), 3.00 (s, 3H), 1.61-1.43 (m, 4H), 1.30 (s, 6H), 1.12 (s, 6H).

### Example 25: Synthesis of Compound 138

### Synthesis of Intermediate B62

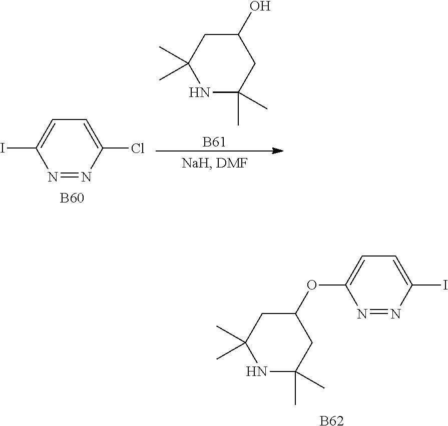

Sodium hydride (150 mg, 3.75 mmol, 60%) was added in portions to a solution of 2,2,6,6-tetramethylpiperidin-4-ol (B61; 641 mg, 4 mmol) in dimethylformamide (10 mL) at 0° C. The resulting mixture was then warmed to 20° C. and stirred for 30 min. 3-Chloro-6-iodopyridazine (B60; 1 g, 4.1 mmol) was then added to the mixture dropwise at 0° C., and the resulting mixture was stirred for additional 21 h at 20° C. The reaction was quenched by the addition of water/ice (40 mL), and extracted with ethyl acetate (40 mL). The combined organic layers were washed with saturated sodium chloride solution (3×50 mL), dried over anhydrous Na₂SO₄, and concentrated under reduced pressure. The residue was purified by silica gel column chromatography, eluting with dichloromethane:methanol (5:1) to afford 3-iodo-6-((2,2,6,6-tetramethylpiperidin-4-yl)oxy)pyridazine (B62; 679 mg) as a solid. LCMS (ES, m/z): 362 [M+H]⁺.

### Synthesis of Intermediate B63

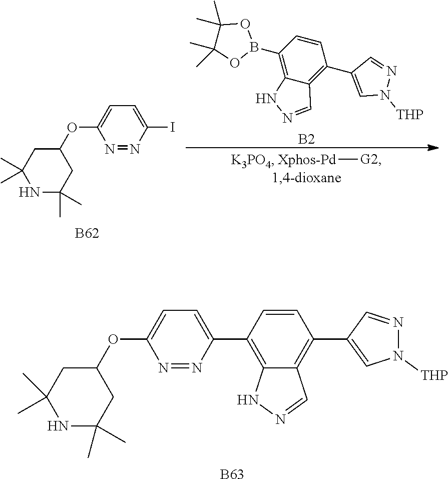

An aqueous solution of potassium triphosphate (2 M, 0.57 mL, 1.14 mmol) was added dropwise to a solution of 3-iodo-6-[(2,2,6,6-tetramethylpiperidin-4-yl)oxy]pyridazine (B62; 165 mg, 0.46 mmol), 4-[1-(oxan-2-yl)pyrazol-4-yl]-7-(4,4,5,5-tetramethyl-1,3,2-dioxaborolan-2-yl)-1H-indazole (B2 from Example 1; 150 mg, 0.381 mmol), and XPhos Pd G2 (30 mg, 0.04 mmol) in 1,4-dioxane (4 mL) at 20° C. under an atmosphere of nitrogen. The resulting mixture was stirred for 3 h at 80° C., and then concentrated under reduced pressure. The residue was purified by silica gel column chromatography, eluting with dichloromethane:methanol (10:1) to afford 4-(1-(tetrahydro-2H-pyran-2-yl)-1H-pyrazol-4-yl)-7-(6-((2,2,6,6-tetramethylpiperidin-4-yl)oxy)pyridazin-3-yl)-1H-indazole (B63; 120 mg) as a solid. LCMS (ES, m/z): 502 [M+H]⁺.

### Synthesis of Compound 138

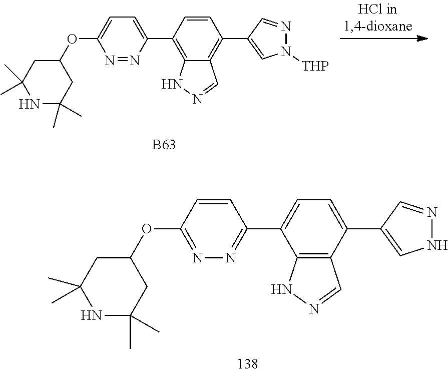

A mixture of 4-[1-(oxan-2-yl)pyrazol-4-yl]-7-[6-[(2,2,6,6-tetramethylpiperidin-4-yl)oxy]pyridazin-3-yl]-1H-indazole (B63; 120 mg, 0.24 mmol) and HCl in 1,4-dioxane (4 mL) was stirred at 20° C. for 30 min, and then concentrated under reduced pressure. The crude product was purified by preparative HPLC (Condition 4, Gradient 1), to afford 4-(1H-pyrazol-4-yl)-7-[6-[(2,2,6,6-tetramethylpiperidin-4-yl)oxy]pyridazin-3-yl]-1H-indazole (Compound 138; 11.3 mg) as a solid. LCMS (ES, m/z):418 [M+H]⁺. ¹H NMR (400 MHz, DMSO-d₆) δ 13.23 (s, 1H), 13.17 (s, 1H), 8.56 (s, 1H), 8.50 (s, 1H), 8.41 (d, J=9.5 Hz, 1H), 8.22 (s, 1H), 8.00 (d, J=7.7 Hz, 1H), 7.52 (d, J=7.6 Hz, 1H), 7.32 (d, J=9.3 Hz, 1H), 5.79 (s, 1H), 2.18-2.06 (m, 2H), 2.00 (s, 1H), 1.41 (s, 1H), 1.33 (d, J=11.6 Hz, 2H), 1.27 (s, 8H), 1.22 (d, J=17.9 Hz, 1H), 1.13 (s, 6H).

### Example 26: Synthesis of Compound 142

### Synthesis of Intermediate B65

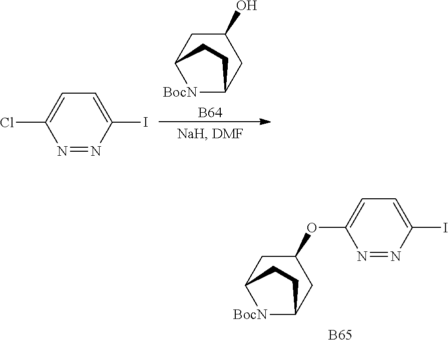

Sodium hydride (0.24 g, 6 mmol) was added in portions to a solution of tert-butyl-3-hydroxy-8-azabicyclo[3.2.1]octane-8-carboxylate (B64; 0.93 g, 4.1 mmol) in dimethylformamide (10 mL) at 0° C., and the resulting mixture was warmed to 20° C. and stirred for 30 min. 3-Chloro-6-iodopyridazine (B60; 1 g, 4.1 mmol) was then added to the mixture dropwise at 0-5° C., and the resulting mixture was stirred for additional 18 h at 20° C. The reaction was quenched by the addition of water/ice (50 mL), and extracted with ethyl acetate (50 mL). The combined organic layers were washed with saturated sodium chloride solution (3×50 mL) and dried over anhydrous Na₂SO₄. The resulting mixture was concentrated under reduced pressure to afford tert-butyl (1R,3R,5S)-3-((6-iodopyridazin-3-yl)oxy)-8-azabicyclo[3.2.1]octane-8-carboxylate (B65; 1.58 g) as a solid. LCMS (ES, m/z): 432 [M+H]⁺.

### Synthesis of Intermediate B66

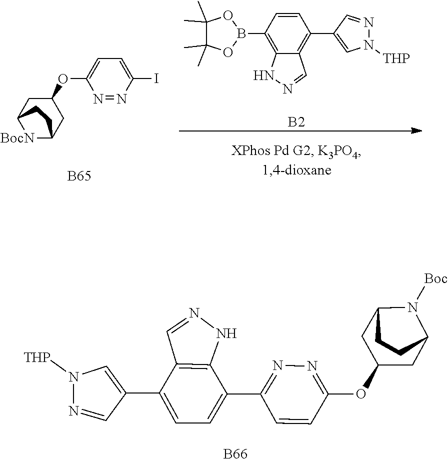

An aqueous solution of K₃PO₄ (2 M, 609 μL, 1.22 mmol) was added dropwise to a solution of tert-butyl-3-[(6-iodopyridazin-3-yl)oxy]-8-azabicyclo[3.2.1]octane-8-carboxylate (B65; 193 mg, 0.45 mmol), 4-[1-(oxan-2-yl)pyrazol-4-yl]-7-(4,4,5,5-tetramethyl-1,3,2-dioxaborolan-2-yl)-1H-indazole (B2 from Example 1; 160 mg, 0.41 mmol) and XPhos Pd G2 (32 mg, 0.041 mmol) in 1,4-dioxane (5 mL) at 20° C. under an atmosphere of nitrogen. The resulting mixture was then heated to 80° C. and stirred for 3 h. The resulting mixture was concentrated under reduced pressure, and purified by silica gel column chromatography eluting with ethyl acetate, to afford B66 (95 mg) as a solid. LCMS (ES, m/z): 572 [M+H]⁺.

### Synthesis of Compound 142

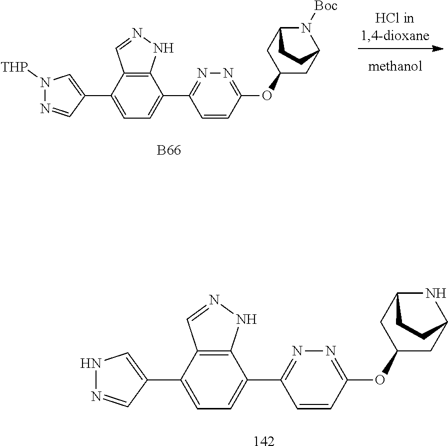

A mixture of tert-butyl (1R,3S,5S)-3-[(6-[4-[1-(oxan-2-yl)pyrazol-4-yl]-1H-indazol-7-yl]pyridazin-3-yl)oxy]-8-azabicyclo[3.2.1]octane-8-carboxylate (95 mg, 0.17 mmol), HCl in 1,4-dioxane (4 mL), and methanol (2 mL) were stirred at 20° C. for 30 min. The resulting mixture was concentrated under reduced pressure, and purified by preparative HPLC (Condition 4, Gradient 3) to afford 7-[6-[(1R,3S,5S)-8-azabicyclo[3.2.1]octan-3-yloxy]pyridazin-3-yl]-4-(1H-pyrazol-4-yl)-1H-indazole (Compound 142; 17.8 mg) as a solid. LCMS (ES, m/z): 388 [M+H]⁺. ¹H NMR (400 MHz, DMSO-d₆) δ 13.21 (d, J=16.9 Hz, 2H), 8.55 (s, 1H), 8.40 (d, J=9.4 Hz, 1H), 8.37 (s, 2H), 8.00 (d, J=7.7 Hz, 1H), 7.51 (d, J=7.6 Hz, 1H), 7.30 (d, J=9.4 Hz, 1H), 5.62 (tt, J=11.0, 6.0 Hz, 1H), 3.54 (t, J=3.3 Hz, 2H), 2.22 (ddd, J=12.4, 6.1, 2.8 Hz, 2H), 1.74 (hept, J=7.2, 6.4 Hz, 4H), 1.63 (td, J=11.6, 3.0 Hz, 2H).

### Example 27: Synthesis of Compound 143

### Synthesis of Intermediate B68

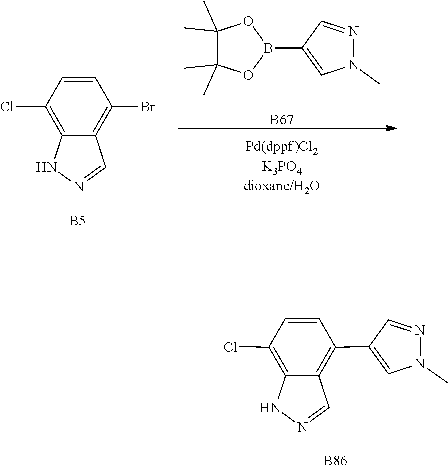

A mixture of 4-bromo-7-chloro-1H-indazole (B5; 1 g, 4.25 mmol, 1 equiv), 1-methyl-4-(4,4,5,5-tetramethyl-1,3,2-dioxaborolan-2-yl)-1H-pyrazole (B67; 1.1 g, 5.1 mmol, 1.2 equiv), K₃PO₄ (2.76 g, 13 mmol, 3 equiv), Pd(dppf)Cl₂—CH₂Cl₂ (177 mg, 0.22 mmol, 0.05 equiv) in dioxane (16 mL) and H₂O (4 mL) was stirred for 4 h at 80° C. The reaction was then quenched by the addition of water/ice (50 mL). The resulting solution was extracted with ethyl acetate (3×50 mL), and the combined organic layers were washed with saturated aqueous NaCl (100 mL), then dried over anhydrous sodium sulfate and filtered. The filtrate was concentrated under vacuum, washed with methanol (20 mL) and filtered, to provide 7-chloro-4-(1-methyl-1H-pyrazol-4-yl)-1H-indazole (B68; 730 mg) as a solid. LCMS (ES, m/z): 233 [M+H]⁺.

### Synthesis of Intermediate B69

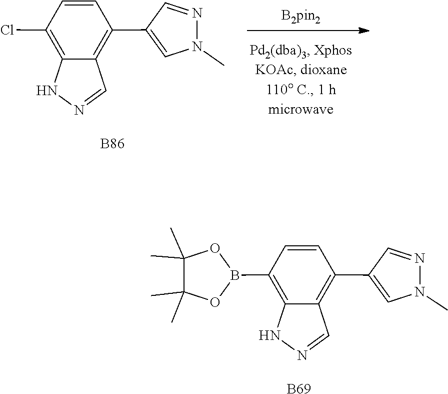

A mixture of 7-chloro-4-(1-methyl-1H-pyrazol-4-yl)-1H-indazole (B68; 300 mg, 1.3 mmol, 1 equiv), B₂(Pin)₂ (591 mg, 2.3 mmol, 1.8 equiv), KOAc (380 mg, 3.9 mmol, 3 equiv), XPhos (184.6 mg, 0.39 mmol, 0.3 equiv), Pd₂(dba)₃.CHCl₃ (107 mg, 0.1 mmol, 0.08 equiv), and dioxane (9 mL) was stirred for 1 h at 110° C. in a 30-mL microwave tube. The solids were then removed by filtration, to obtain crude B69 that was added to the next step directly without further purification. LCMS (ES, m/z): 325 [M+H]⁺.

### Synthesis of Intermediate B71

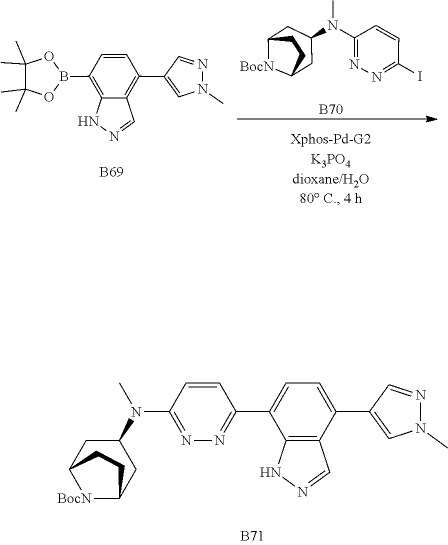

A mixture of 4-(1-methyl-1H-pyrazol-4-yl)-7-(4,4,5,5-tetramethyl-1,3,2-dioxaborolan-2-yl)-1H-indazole (B69; 300 mg, 0.92 mmol, 1 equiv), tert-butyl (1R,3r,5S)-3-((6-iodopyridazin-3-yl)(methyl)amino)-8-azabicyclo[3.2.1]octane-8-carboxylate (B70; 493 mg, 1.11 mmol, 1.2 equiv), K₃PO₄ (589 mg, 2.8 mmol, 3 equiv), XPhos palladium(II) biphenyl-2-amine chloride (36.4 mg, 0.05 mmol, 0.05 equiv), dioxane (16 mL), and H₂O (4 mL) was stirred for 16 h at 80° C. The reaction was then quenched by the addition of water/ice (2 mL), and the resulting solution was extracted with ethyl acetate (3×10 mL). The combined organic layers were washed with saturated aqueous NaCl (50 mL). The solid was dried in an oven under reduced pressure. The solids were filtered out. The resulting mixture was concentrated under vacuum, and purified by silica gel column chromatography eluting with ethyl acetate/petroleum ether (7:3) to provide tert-butyl (1R,3r,5S)-3-(methyl(6-(4-(1-methyl-1H-pyrazol-4-yl)-1H-indazol-7-yl)pyridazin-3-yl)amino)-8-azabicyclo[3.2.1]octane-8-carboxylate (B71; 210 mg) as a solid. LCMS (ES, m/z): 515 [M+H]⁺.

### Synthesis of Compound 143

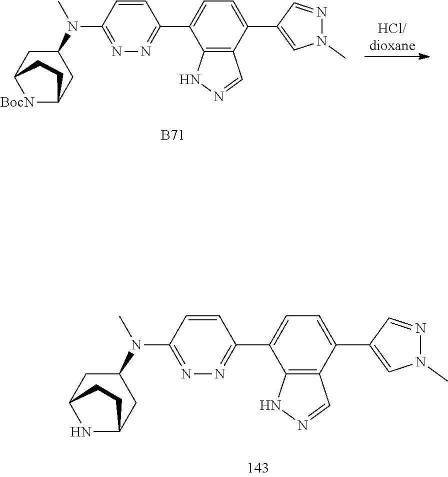

A mixture of tert-butyl (1R,3r,5S)-3-(methyl(6-(4-(1-methyl-1H-pyrazol-4-yl)-1H-indazol-7-yl)-pyridazin-3-yl)-amino)-8-azabicy-clo[3.2.1]octane-8-carboxylate (B71; 210 mg, 0.41 mmol, 1 equiv), dioxane (2 mL), and 4 M HCl in dioxane (2 mL) was stirred for 3 h at room temperature, in a 50-mL round-bottom flask that was purged and maintained with an inert atmosphere of nitrogen. The reaction mixture was then cooled with a water/ice bath and quenched by the addition of methanol. The resulting mixture was concentrated under vacuum and purified by preparative HPLC (Condition 4, Gradient 5) to provide 6-[6-[8-azabicyclo[3.2.1]-octan-3-yl(methyl)amino]-pyridazin-3-yl]-3-(1H-pyrazol-4-yl)-1H-pyridin-2-one (Compound 143; 5.6 mg) as a solid. LCMS (ES, m/z): 415 [M+H]⁺. ¹H NMR (400 MHz, DMSO-d6) δ 13.09 (s, 1H), 8.48 (dd, J=3.2, 1.1 Hz, 2H), 8.18-8.08 (m, 2H), 7.89 (d, J=7.8 Hz, 1H), 7.43 (d, J=7.6 Hz, 1H), 7.24 (d, J=9.8 Hz, 1H), 5.04 (dd, J=11.6, 6.1 Hz, 1H), 3.96 (s, 3H), 3.51 (s, 2H), 2.97 (s, 3H), 2.16 (s, 1H), 1.88-1.74 (m, 3H), 1.76 (s, 4H), 1.56 (dq, J=12.2, 4.0, 3.4 Hz, 2H).

### Example 28: Synthesis of Compound 146

### Synthesis of Intermediate B73

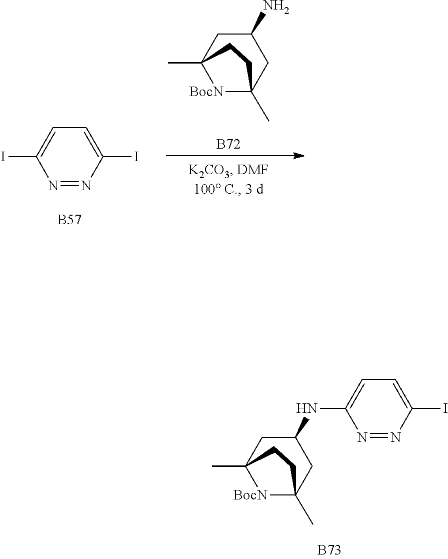

A mixture of 3,6-diiodopyridazine (B57; 1 g, 2.95 mmol), tert-butyl-3-amino-1,5-dimethyl-8-azabicyclo[3.2.1]octane-8-carboxylate (B72; 751 mg, 2.95 mmol) and K₂CO₃ (1.22 g, 8.86 mmol) in dimethylformamide (15 mL) was stirred for 3 days at 100° C. The mixture was then cooled to 20° C. and poured onto brine (100 mL). The resulting mixture was extracted with ethyl acetate (3×100 mL), and the combined organic layers were washed with brine (2×100 mL), dried over anhydrous Na₂SO₄, filtered, and concentrated under reduced pressure. The residue was purified by silica gel column chromatography, eluting with petroleum ether/ethyl acetate (1:1) to afford tert-butyl (1R,3S,5S)-3-[(6-iodopyridazin-3-yl)amino]-1,5-dimethyl-8-azabicyclo[3.2.1]octane-8-carboxylate (B73; 300 mg) as an oil. LCMS (ES, m/z): 459 [M+H]⁺.

### Synthesis of Intermediate B74

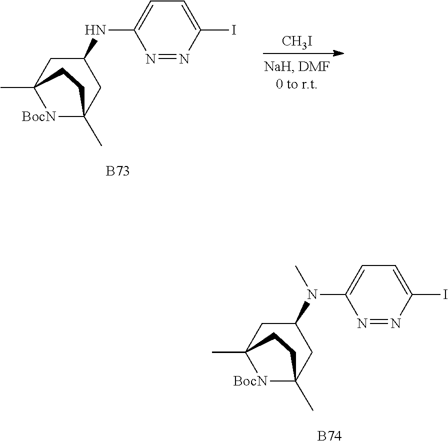

Sodium hydride (46 mg, 1.16 mmol, 60%) was added in portions to a solution of tert-butyl-3-[(6-iodopyridazin-3-yl)amino]-1,5-dimethyl-8-azabicyclo[3.2.1]octane-8-carboxylate (B73; 360 mg, 0.77 mmol) in dimethylformamide (5 mL) at 0° C., and the resulting mixture was stirred for 30 min at 0° C. Methyl iodide (73 mg, 1.155 mmol) was then added to the reaction dropwise, and the mixture was warmed to 25° C. and stirred for additional 30 min. The reaction mixture was poured onto water (20 mL) and extracted with ethyl acetate (3×20 mL). The combined organic layers were washed with brine (2×20 mL), dried over anhydrous Na₂SO₄, filtered, and concentrated under reduced pressure. The residue was purified by silica gel column chromatography, eluting with petroleum ether/ethyl acetate (10:1) to afford tert-butyl-3-[(6-iodopyridazin-3-yl)(methyl)amino]-1,5-dimethyl-8-azabicyclo[3.2.1]octane-8-carboxylate (B74; 150 mg) as an oil. LCMS (ES, m/z): 473 [M+H]⁺.

### Synthesis of Intermediate B75

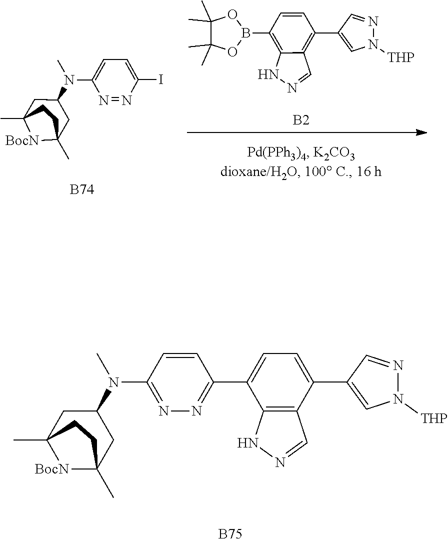

A solution of tert-butyl-3-[(6-iodopyridazin-3-yl)(methyl)amino]-1,5-dimethyl-8-azabicyclo[3.2.1]octane-8-carboxylate (B74; 165 mg, 0.34 mmol), 4-[1-(oxan-2-yl)pyrazol-4-yl]-7-(4,4,5,5-tetramethyl-1,3,2-dioxaborolan-2-yl)-1H-indazole (B2 from Example 1; 135 mg, 0.34 mmol), Pd(PPh₃)₄ (27 mg, 0.023 mmol) and K₂CO₃ (218 mg, 1.58 mmol) in dioxane (5 mL) was stirred for 4 h at 80° C. under a nitrogen atmosphere. The mixture was then cooled to 20° C. and concentrated under vacuum. The residue was purified by silica gel column chromatography, eluting with petroleum ether/ethyl acetate (1:1) to afford tert-butyl-1,5-dimethyl-3-[methyl(6-[4-[1-(oxan-2-yl)pyrazol-4-yl]-1H-indazol-7-yl]pyridazin-3-yl)amino]-8-azabicyclo[3.2.1]octane-8-carboxylate (B75; 160 mg) as a solid. LCMS (ES, m/z): 613 [M+H]⁺.

### Synthesis of Compound 146

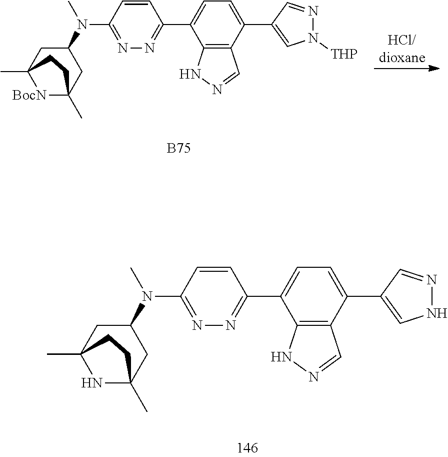

A mixture of tert-butyl-1,5-dimethyl-3-[methyl(6-[4-[1-(oxan-2-yl)pyrazol-4-yl]-1H-indazol-7-yl]pyridazin-3-yl)amino]-8-azabicyclo[3.2.1]octane-8-carboxylate (B75; 160 mg, 0.26 mmol) and HCl in 1,4-dioxane (5 mL) was stirred for 30 min at 25° C., and the resulting mixture was concentrated under reduced pressure. The crude product was purified by preparative HPLC (Condition 4, Gradient 6) to afford N,1,5-trimethyl-N-[6-[4-(1H-pyrazol-4-yl)-1H-indazol-7-yl]pyridazin-3-yl]-8-azabicyclo[3.2.1]octan-3-amine (Compound 146; 15.2 mg) as a solid. LCMS (ES, m/z): 429 [M+H]⁺. ¹H NMR (400 MHz, DMSO-d6) δ 13.07 (s, 1H), 8.51 (s, 1H), 8.33 (s, 1H), 8.16 (d, J=9.8 Hz, 2H), 7.89 (d, J=7.7 Hz, 1H), 7.47 (d, J=7.6 Hz, 1H), 7.26 (d, J=9.8 Hz, 1H), 5.08 (s, 1H), 2.98 (s, 3H), 1.87 (d, J=7.6 Hz, 2H), 1.57 (t, J=11.2 Hz, 6H), 1.21 (s, 6H).

### Example 29: Synthesis of Compound 150

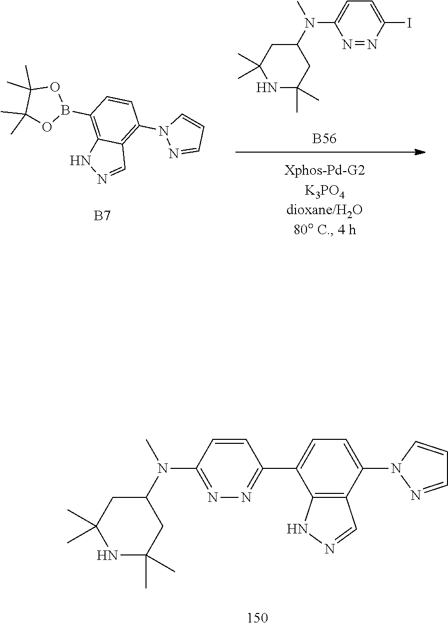 [SMILES: CC1(C)OB(c2ccc(-n3cccn3)c3cn[nH]c23)OC1(C)C ; CN(c1ccc(I)nn1)C1CC(C)(C)NC(C)(C)C1 ; CN(c1ccc(-c2ccc(-n3cccn3)c3cn[nH]c23)nn1)C1CC(C)(C)NC(C)(C)C1]

A mixture of 4-(pyrazol-1-yl)-7-(4,4,5,5-tetramethyl-1,3,2-dioxaborolan-2-yl)-1H-indazole (B7 from Example 2; 250 mg, 0.81 mmol, 1 equiv), 6-iodo-N-methyl-N-(2,2,6,6-tetramethylpiperidin-4-yl)pyridazin-3-amine (B56 from Example 24; 362 mg, 0.97 mmol, 1.2 equiv), K₃PO₄ (513 mg, 2.42 mmol, 3 equiv), XPhos palladium(II) biphenyl-2-amine chloride (63.4 mg, 0.081 mmol, 0.1 equiv), dioxane (8 mL), and water (2 mL) in a 20-mL vial was purged and maintained under an inert atmosphere of nitrogen, and then heated to 80° C. for 4 h. The resulting mixture was concentrated under vacuum and purified by silica gel column chromatography eluting with dichloromethane/methanol (10:1). The resulting product was further purified by preparative HPLC (Condition 3) to provide N-methyl-6-[4-(pyrazol-1-yl)-1H-indazol-7-yl]-N-(2,2,6,6-tetramethylpiperidin-4-yl)pyridazin-3-amine (Compound 150; 2.9 mg) as a solid. LCMS (ES, m/z): 431 [M+H]⁺. 1H NMR: (400 MHz, DMSO-d₆, ppm) δ 13.26 (s, 1H), 8.70 (d, J=2.5 Hz, 1H), 8.63 (d, J=1.7 Hz, 1H), 8.20 (d, J=9.8 Hz, 1H), 7.99 (d, J=8.0 Hz, 1H), 7.92 (d, J=1.7 Hz, 1H), 7.62 (d, J=8.0 Hz, 1H), 7.29 (d, J=9.8 Hz, 1H), 6.69-6.64 (m, 1H), 5.11 (s, 1H), 3.02 (s, 4H), 1.59 (s, 5H), 1.33 (s, 6H), 1.15 (s, 6H).

### Example 30: Synthesis of Compound 132

### Synthesis of Intermediate B78

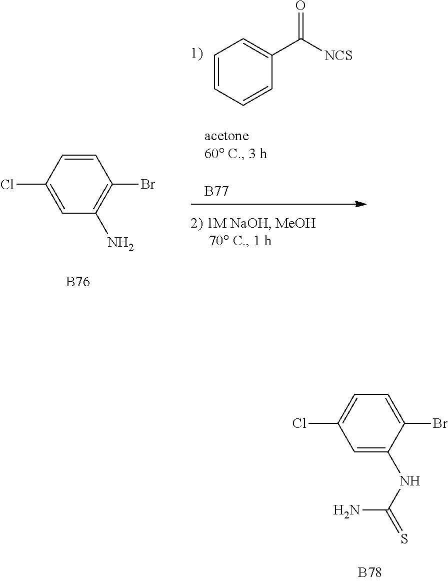

Benzoyl isothiocyanate (B77; 17.4 g, 107 mmol) was added dropwise to a solution of 2-bromo-5-chloroaniline (B76; 20 g, 97) in acetone (200 mL), and the reaction mixture was heated to reflux for 3 h, then poured into water-ice, and stirred for an additional 30 min. The precipitate was collected by filtration and washed with more water. The crude material was then dissolved in methanol (200 mL) and treated with sodium hydroxide (1N, 50 mL), and heated to 80° C. for 2 h. The mixture was then cooled, and poured into water-ice, and the pH of the mixture was neutralized using 1N HCl. The precipitate was collected by filtration and dried to afford 2-bromo-5-chlorophenylthiourea (B78; 22.1). LCMS (ES, m/z): 264.85 [M+H]⁺.

### Synthesis of Intermediate B79

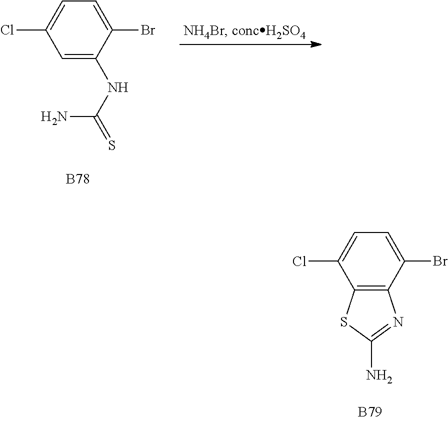 [SMILES: NC(=S)Nc1cc(Cl)ccc1Br ; Nc1nc2c(Br)ccc(Cl)c2s1]

Ammonium bromide (1.83 g, 18.8 mmol) was added to a solution of 2-bromo-5-chlorophenylthiourea (B78; 5 g, 18.8 mmol) in concentrated H₂SO₄ (15 mL) over a period of 1 h, and the reaction mixture was heated to 100° C. for 2 h. The reaction mixture was then cooled to room temperature and then poured into ice water (150 mL). The pH value of the mixture was neutralized using aqueous ammonium hydroxide solution, and the precipitate was collected by filtration, washed with water and dried in vacuum to afford 4-bromo-7-chloro-1,3-benzothiazol-2-amine (B79; 4 g) as a solid. LCMS (ES, m/z): 262.85 [M+H]⁺. ¹H NMR (400 MHz, DMSO-d₆) δ 8.13 (s, 2H), 7.45 (d, J=8.4 Hz, 1H), 7.02 (d, J=8.5 Hz, 1H).

### Synthesis of Intermediate B80

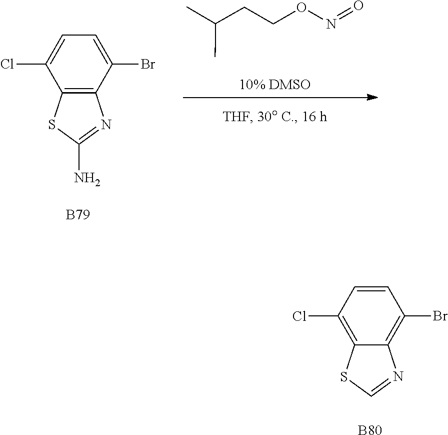

A solution of 4-bromo-7-chloro-1,3-benzothiazol-2-amine (3.9 g, 14.8 mmol) in tetrahydrofuran (12 mL) was added dropwise over 20 min to a solution of isopentyl nitrite (2.6 g, 22.2 mmol) and dimethylsulfoxide (115.63 mg, 1.48 mmol) in THF (28 mL). The mixture was stirred at 30° C. for 3 h, and then diluted with ethyl acetate (50 mL) and water (60 mL), and the aqueous layer was extracted with ethyl acetate (2×50 mL). The combined organic layers were washed with saturated brine (100 mL), dried over anhydrous sodium sulfate and concentrated to give a residue. The residue was purified by silica gel column chromatography eluting with 20% ethyl acetate in petroleum ether to afford 4-bromo-7-chloro-1,3-benzothiazole (B80; 2.2 g) as a solid. ¹H NMR (400 MHz, DMSO-d₆) δ 9.58 (s, 1H), 7.84 (d, J=8.3 Hz, 1H), 7.56 (d, J=8.3 Hz, 1H).

### Synthesis of Intermediate B81

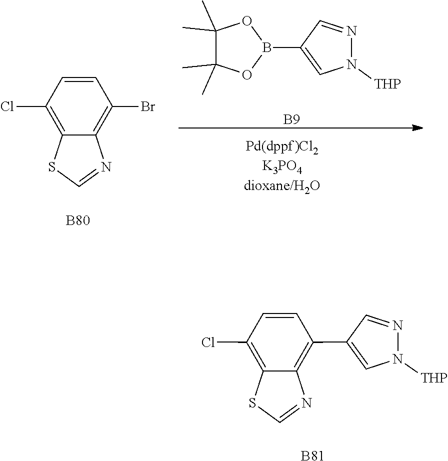

Tripotassium phosphate (5.38 g, 25 mmol) and Pd(dppf)Cl₂ (309 mg, 0.42 mmol) were added to a solution of 4-bromo-7-chloro-1,3-benzothiazole (B80; 2.1 g, 8 mmol) and 1-(oxan-2-yl)-4-(4,4,5,5-tetramethyl-1,3,2-dioxaborolan-2-yl)pyrazole (2.59 g, 9 mmol) in dioxane (16 mL) and H₂O (4 mL), and the mixture was stirred overnight at 100° C. under a nitrogen atmosphere. The mixture was then concentrated under reduced pressure, diluted with ethyl acetate (25 mL) and water (30 mL), and the aqueous layer was extracted with ethyl acetate (2×25 mL). The combined organic layers were washed with saturated brine (40 mL), dried over anhydrous sodium sulfate and concentrated to give a residue. The residue was purified by silica gel column chromatography eluting with 35% ethyl acetate in petroleum ether to afford 7-chloro-4-[1-(oxan-2-yl)pyrazol-4-yl]-1,3-benzothiazole (B81; 2.1 g) as a solid. LCMS (ES, m/z): 320.05 [M+H]⁺.

### Synthesis of Intermediate B82

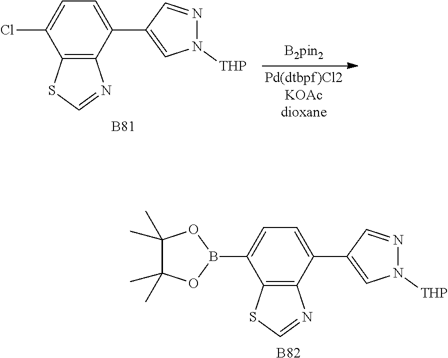

Potassium acetate (460 mg, 4.69 mmol) and Pd(dtbpf)Cl₂ (51 mg, 0.078 mmol) were added to a solution of 7-chloro-4-[1-(oxan-2-yl)pyrazol-4-yl]-1,3-benzothiazole (B81; 500 mg, 1.56 mmol) and bis(pinacolato)diboron (437 mg, 1.72 mmol) in dioxane (5 mL), and the mixture was stirred overnight at 100° C. under a nitrogen atmosphere. The resulting mixture was concentrated under reduced pressure and diluted with ethyl acetate (10 mL) and water (15 mL), and the aqueous layer was extracted with ethyl acetate (2×10 mL). The combined organic layers were washed with saturated brine (20 mL), dried over anhydrous sodium sulfate and concentrated to give crude 4-[1-(oxan-2-yl)pyrazol-4-yl]-7-(4,4,5,5-tetramethyl-1,3,2-dioxaborolan-2-yl)-1,3-benzothiazole (B82; 1 g) as an oil. LCMS (ES, m/z): 412.10 [M+H]⁺.

### Synthesis of Intermediate B83

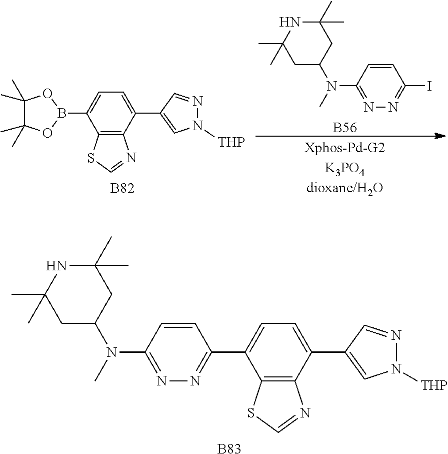

Tripotassium phosphate (510 mg, 2.4 mmol) and XPhos Pd G2 (31 mg, 0.04 mmol) were added to a solution of 6-iodo-N-methyl-N-(2,2,6,6-tetramethylpiperidin-4-yl)pyridazin-3-amine (B56; 300 mg, 0.8 mmol) and 4-[1-(oxan-2-yl)pyrazol-4-yl]-7-(4,4,5,5-tetramethyl-1,3,2-dioxaborolan-2-yl)-1,3-benzothiazole (B82; 989 mg, 2.4 mmol) in dioxane (2 mL) and H₂O (8 mL), and the mixture was stirred overnight at 100° C. under a nitrogen atmosphere. The resulting mixture was concentrated under reduced pressure, diluted with ethyl acetate (15 mL) and water (12 mL), and the aqueous layer was extracted with ethyl acetate (2×15 mL). The organic layers were combined and washed with saturated brine (30 mL), dried over anhydrous sodium sulfate and concentrated to give a residue. The residue was purified by silica gel column chromatography eluting with 40% ethyl acetate in petroleum ether to afford N-methyl-6-[4-[1-(oxan-2-yl)pyrazol-4-yl]-1,3-benzothiazol-7-yl]-N-(2,2,6,6-tetramethylpiperidin-4-yl)pyridazin-3-amine (B83; 170 mg) as a solid. LCMS (ES, m/z): 532.25 [M+H]⁺.

### Synthesis of Compound 132

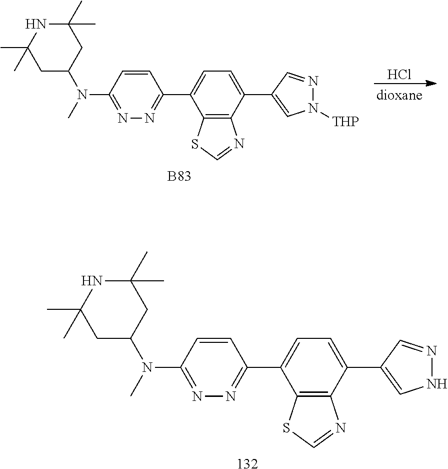

A solution of HCl in 1,4-dioxane (2 mL) was added to N-methyl-6-[4-[1-(oxan-2-yl)pyrazol-4-yl]-1,3-benzothiazol-7-yl]-N-(2,2,6,6-tetramethylpiperidin-4-yl)pyridazin-3-amine (B83; 170 mg, 0.32 mmol) in dioxane (2 mL), and the mixture was stirred at room temperature for 2h. The mixture was then concentrated under vacuum, and the crude product was purified by preparative HPLC (Condition 1) to afford N-methyl-6-[4-(1H-pyrazol-4-yl)-1,3-benzothiazol-7-yl]-N-(2,2,6,6-tetramethylpiperidin-4-yl)pyridazin-3-amine (Compound 132; 29 mg) as a solid. LCMS (ES, m/z): 448.20 [M+H]⁺. ¹H NMR (400 MHz, DMSO-d6) δ 13.10 (s, 1H), 9.51 (s, 1H), 8.63 (s, 1H), 8.47 (s, 1H), 8.27 (d, J=9.8 Hz, 1H), 8.11 (d, J=8.1 Hz, 1H), 7.99 (d, J=7.9 Hz, 1H), 7.27 (d, J=9.7 Hz, 1H), 5.01 (s, 1H), 3.01 (s, 3H), 1.55 (dd, J=12.0, 3.6 Hz, 2H), 1.46 (t, J=12.0 Hz, 2H), 1.29 (s, 6H), 1.11 (s, 6H).

### Example 31: Synthesis of Compound 163

### Synthesis of Intermediate B85

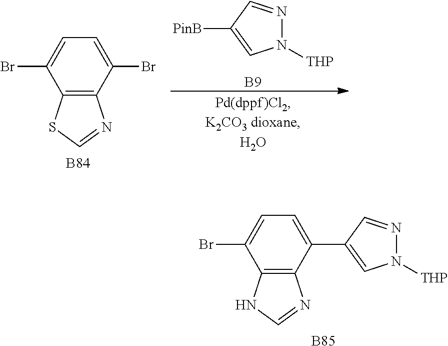

Potassium carbonate (3 g, 21.8 mmol) and Pd(dppf)Cl₂.CH₂Cl₂ (0.3 g, 0.36 mmol) were added in portions to a mixture of 4,7-dibromo-1H-1,3-benzodiazole (B84; 2 g, 7.25 mmol) and 4-boranyl-1-(oxan-2-yl) pyrazole (B9; 1.19 g, 7.25 mmol) in dioxane (20 mL) and H₂O (10 mL) at room temperature under a nitrogen atmosphere, and the resulting mixture was stirred overnight at 80° C. The mixture was then extracted with ethyl acetate (2×50 mL), and the combined organic layers were washed with brine (2×100 mL), dried over anhydrous Na₂SO₄, filtered, and concentrated under reduced pressure. The residue was purified by silica gel column chromatography, eluting with petroleum ether/ethyl acetate (13:7), to afford 4-bromo-7-[1-(oxan-2-yl) pyrazol-4-yl]-3H-1,3-benzodiazole (B85; 700 mg) as a solid. LCMS (ES, m/z): 347 [M+H]⁺.

### Synthesis of Intermediate B86

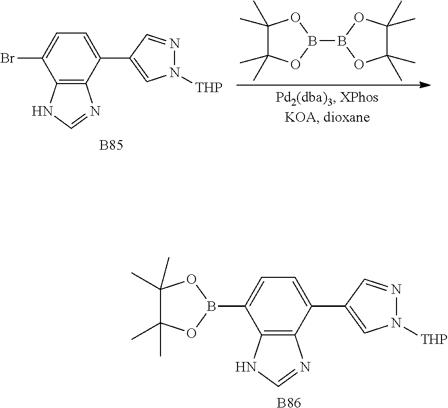

Potassium acetate (254 mg, 2.59 mmol), XPhos (41.2 mg, 0.086 mmol) and Pd₂(dba)₃ (39.6 mg, 0.043 mmol) were added in portions to a mixture of 4-bromo-7-[1-(oxan-2-yl) pyrazol-4-yl]-3H-1,3-benzodiazole (B85; 300 mg, 0.86 mmol) and bis(pinacolato)diboron (329 mg, 1.3 mmol) in dioxane (3 mL) at room temperature under a nitrogen atmosphere, and the resulting mixture was then heated to 80° C. for 3 h. The resulting mixture was filtered and concentrated under reduced pressure, and the residue was purified by silica gel column chromatography, eluting with petroleum ether/ethyl acetate (3:2), to afford 4-[1-(oxan-2-yl) pyrazol-4-yl]-7-(4,4,5,5-tetramethyl-1,3,2-dioxaborolan-2-yl)-1H-1,3-benzodiazole (B86; 160 mg) as a solid. LCMS (ES, m/z): 395 [M+H]⁺.

### Synthesis of Intermediate B87

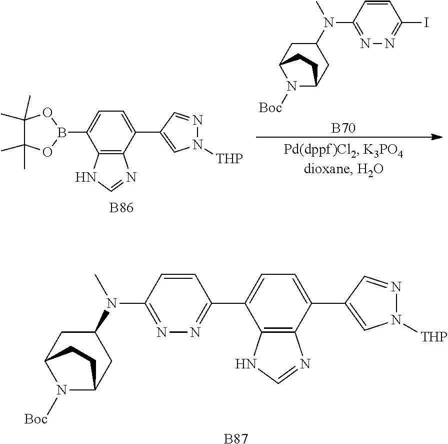

Tripotassium phosphate (234 mg, 1.1 mmol) and Pd(dppf)Cl₂ (21.3 mg, 0.018 mmol) were added in portions to a mixture of 4-[1-(oxan-2-yl) pyrazol-4-yl]-7-(4,4,5,5-tetramethyl-1,3,2-dioxaborolan-2-yl)-1H-1,3-benzodiazole (B86; 145 mg, 0.37 mmol) and tert-butyl (1R,3S,5S)-3-[(6-iodopyridazin-3-yl) (methyl)amino]-8-azabicyclo [3.2.1] octane-8-carboxylate (B70; 245 mg, 0.55 mmol) in dioxane (2 mL) and H₂O (1 mL) at room temperature under a nitrogen atmosphere. The resulting mixture was stirred overnight at 80° C., then filtered. The filtrate was concentrated under reduced pressure, and purified by silica gel column chromatography, eluting with petroleum ether/ethyl acetate (13:7) to afford tert-butyl (1R,3S,5S)-3-[methyl(6-[7-[1-(oxan-2-yl) pyrazol-4-yl]-3H-1,3-benzodiazol-4-yl] pyridazin-3-yl) amino]-8-azabicyclo [3.2.1] octane-8-carboxylate (B87; 120 mg) as a solid. LCMS (ES, m/z): 585 [M+H]⁺.

### Synthesis of Compound 163

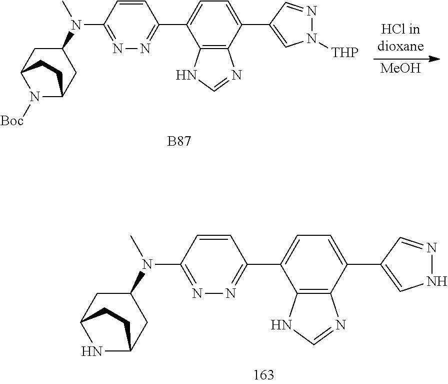

A solution of HCl in 1,4-dioxane (3 mL) was added dropwise to a solution of (1R,3S,5S)—N-methyl-N-(6-[7-[1-(oxan-2-yl) pyrazol-4-yl]-3H-1,3-benzodiazol-4-yl] pyridazin-3-yl)-8-azabicyclo [3.2.1] octan-3-amine (B87; 120 mg, 0.25 mmol) in methanol (3 mL) at room temperature under a nitrogen atmosphere, and the resulting mixture was stirred for 1h. The mixture was then filtered, and the filtrate was concentrated under reduced pressure. The residue was purified by preparative HPLC (Condition 4, Gradient 2), to afford (1R,3S,5S)—N-methyl-N-[6-[7-(1H-pyrazol-4-yl)-3H-1,3-benzodiazol-4-yl] pyridazin-3-yl]-8-azabicyclo [3.2.1] octan-3-amine (Compound 163; 44.2 mg) as a solid. LCMS (ES, m/z): 401 [M+H]⁺. ¹H NMR (400 MHz, DMSO-d6) δ 12.99 (s, 1H), 12.51 (s, 1H), 8.69 (s, 1H), 8.40 (s, 1H), 8.22 (s, 1H), 8.13 (d, J=9.8 Hz, 1H), 7.75 (d, J=8.0 Hz, 1H), 7.61 (d, J=7.9 Hz, 1H), 7.24 (d, J=9.8 Hz, 1H), 5.04 (s, 1H), 3.52 (s, 2H), 2.96 (s, 3H), 1.82 (t, J=11.7 Hz, 2H), 1.76 (s, 4H), 1.56 (d, J=11.6 Hz, 2H), 1.24 (s, 1H).

### Example 32: Synthesis of Compound 164

### Synthesis of Intermediate B89

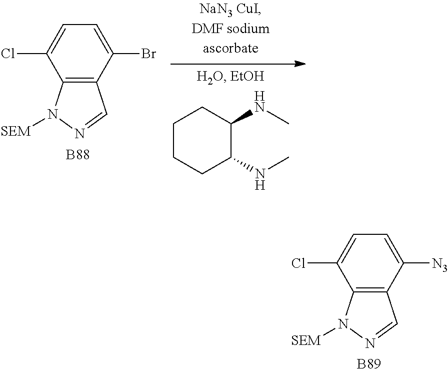

A mixture of 4-bromo-7-chloro-1-[[2-(trimethylsilyl)ethoxy]methyl]indazole (B88; 1 g, 2.71 mmol), NaN₃ (352 mg, 5.42 mmol), sodium ascorbate (108 mg, 0.54 mmol), copper iodide (52 mg, 0.27 mmol) and trans-N,N-dimethylcyclohexane-1,2-diamine (58 mg, 0.41 mmol) in H₂O (6 mL) and ethanol (14 mL) was stirred for 1 h at 100° C. under a nitrogen atmosphere. The mixture was then cooled to 20° C., and poured into water (100 mL). The resulting mixture was extracted with ethyl acetate (3×100 mL), dried over anhydrous Na₂SO₄, filtered, and concentrated under reduced pressure. The residue was purified by silica gel column chromatography, eluting with petroleum ether/ethyl acetate (10:1) to afford 4-azido-7-chloro-1-[[2-(trimethylsilyl)ethoxy]methyl]indazole (B89; 550 mg) as an oil. LCMS (ES, m/z): 324 [M+H]⁺.

### Synthesis of Intermediate B90

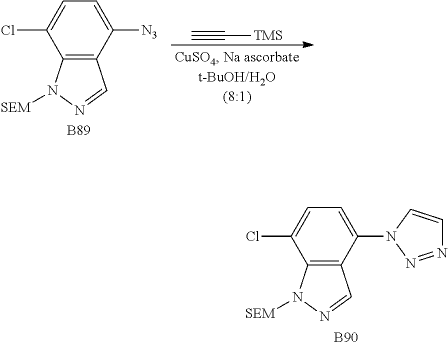

A solution of 4-azido-7-chloro-1-[[2-(trimethylsilyl)ethoxy]methyl]indazole (B89; 500 mg, 1.51 mmol), trimethylsilylacetylene (223 mg, 2.27 mmol), CuSO₄ (24 mg, 0.15 mmol) and sodium ascorbate (60 mg, 0.3 mmol) in tert-butanol (8 mL) and H₂O (2 mL) was stirred for 16 h at 100° C. under a nitrogen atmosphere. The mixture was then cooled to 20° C. and poured into water (30 mL). The resulting mixture was extracted with ethyl acetate (3×30 mL), dried over anhydrous Na₂SO₄, filtered, and concentrated under reduced pressure. The crude product (B90) was used in the next step directly without further purification. LCMS (ES, m/z): 350 [M+H]⁺.

### Synthesis of Intermediate B91

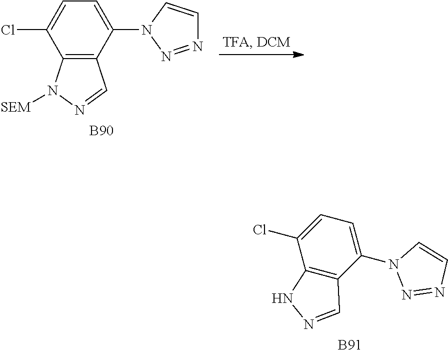

A mixture of 7-chloro-4-(1,2,3-triazol-1-yl)-1-[[2-(trimethylsilyl)ethoxy]methyl]indazole (B90; crude) and trifluoroacetic acid (1 mL) in dichloromethane (3 mL) was stirred for 30 min at 25° C. The resulting mixture was then concentrated under reduced pressure, and purified by reverse phase flash chromatography on a C18 silica gel column, eluting with water (10 mmol/L NH₄HCO₃) and acetonitrile (30% held for 2 min, then increased to 55% over 20 min), to afford 7-chloro-4-(1,2,3-triazol-1-yl)-1H-indazole (B91; 110 mg) as a solid. LCMS (ES, m/z): 220 [M+H]⁺.

### Synthesis of Intermediate B92

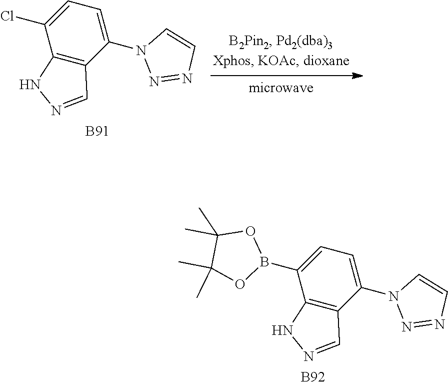 [SMILES: "B91" ; Clc1ccc(-n2ccnn2)c2cn[nH]c12 ; "B92" ; CC1(C)OB(c2ccc(-n3ccnn3)c3cn[nH]c23)OC1(C)C]

A solution of 7-chloro-4-(1,2,3-triazol-1-yl)-1H-indazole (B91; 100 mg, 0.45 mmol), 4,4,5,5-tetramethyl-2-(tetramethyl-1,3,2-dioxaborolan-2-yl)-1,3,2-dioxaborolane (B₂Pin₂; 227 mg, 0.89 mmol), Pd₂(dba)₃.CHCl₃ (23 mg, 0.022 mmol), XPhos (21 mg, 0.045 mmol) and potassium acetate (132 mg, 1.34 mmol) in dioxane (3 mL) was irradiated with microwave for 2 h at 110° C. under a nitrogen atmosphere. The resulting mixture was concentrated under vacuum, and purified by silica gel column chromatography, eluting with petroleum ether/ethyl acetate (5:1) to afford 7-(4,4,5,5-tetramethyl-1,3,2-dioxaborolan-2-yl)-4-(1,2,3-triazol-1-yl)-1H-indazole (B92; 110 mg) as a solid. LCMS (ES, m/z): 312 [M+H]⁺.

### Synthesis of Intermediate B93

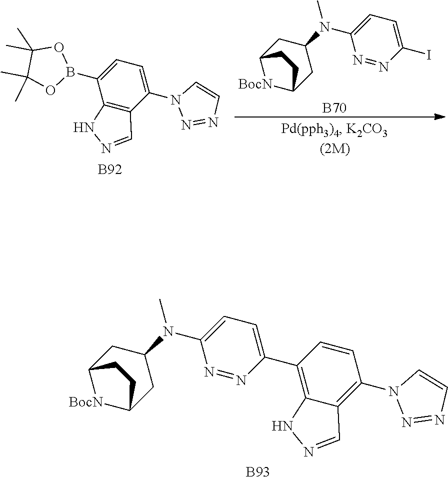

A solution of 7-(4,4,5,5-tetramethyl-1,3,2-dioxaborolan-2-yl)-4-(1,2,3-triazol-1-yl)-1H-indazole (B92; 110 mg, 0.35 mmol), tert-butyl (1R,3S,5S)-3-[(6-iodopyridazin-3-yl)(methyl)amino]-8-azabicyclo[3.2.1]octane-8-carboxylate (B70; 185 mg, 0.42 mmol), Pd(PPh₃)₄ (40 mg, 0.035 mmol) and K₂CO₃ (2M; 0.52 mL, 1.04 mmol) in dioxane (3 mL) was stirred for 16 h at 100° C. under a nitrogen atmosphere. The mixture was then cooled to 25° C. and concentrated under vacuum. The residue was purified by silica gel column chromatography, eluting with petroleum ether/ethyl acetate (1:1) to afford tert-butyl (1R,3S,5S)-3-[methyl([6-[4-(1,2,3-triazol-1-yl)-1H-indazol-7-yl]pyridazin-3-yl])amino]-8-azabicyclo[3.2.1]octane-8-carboxylate (B93; 45 mg) as a solid. LCMS (ES, m/z): 502 [M+H]⁺.

### Synthesis of Compound 164

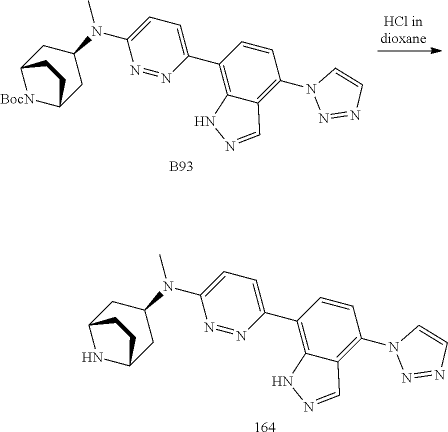

A mixture of tert-butyl (1R,3R,5S)-3-[methyl([6-[4-(1,2,3-triazol-1-yl)-1H-indazol-7-yl]pyridazin-3-yl])amino]-8-azabicyclo[3.2.1]octane-8-carboxylate (B93; 45 mg, 0.088 mmol) and HCl in 1,4-dioxane (2 mL) was stirred for 30 min at 25° C. The resulting mixture was concentrated under reduced pressure, and purified by preparative HPLC (Condition 4, Gradient 3), to afford (1R,3R,5S)—N-methyl-N-[6-[4-(1,2,3-triazol-1-yl)-1H-indazol-7-yl]pyridazin-3-yl]-8-azabicyclo[3.2.1]octan-3-amine (Compound 164; 7 mg) as a solid. LCMS (ES, m/z): 402 [M+H]⁺. 1H NMR (400 MHz, DMSO-d₆, ppm) δ 13.48 (s, 1H), 9.06 (d, J=1.2 Hz, 1H), 8.53 (s, 1H), 8.23 (d, J=9.8 Hz, 1H), 8.12-8.05 (m, 2H), 7.74 (d, J=7.9 Hz, 1H), 7.29 (d, J=9.8 Hz, 1H), 5.12 (s, 1H), 3.58 (s, 2H), 3.00 (s, 3H), 1.93-1.82 (m, 2H), 1.80 (s, 4H), 1.60 (dd, J=13.0, 5.4 Hz, 2H).

### Example 33: Synthesis of Compound 144

### Synthesis of Intermediate B95

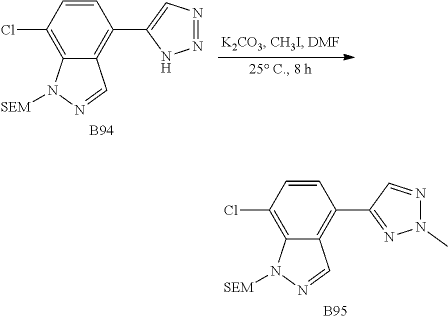

Methyl iodide (143 mg, 1 mmol) was added dropwise to a mixture of 7-chloro-4-(3H-1,2,3-triazol-4-yl)-1-[[2-(trimethylsilyl)ethoxy]methyl]indazole (B94; 300 mg, 0.84 mmol) and potassium carbonate (232 mg, 1.68 mmol) in dimethylformamide (3 mL) at 0° C., and the resulting mixture was stirred for 8 h at 25° C., then poured into water (30 mL) and extracted with ethyl acetate (2×30 mL). The combined organic layers were washed with brine (3×30 mL), dried over anhydrous sodium sulfate, filtered, and concentrated under reduced pressure, to afford 7-chloro-4-(2-methyl-1,2,3-triazol-4-yl)-1-[[2-(trimethylsilyl)ethoxy]methyl]indazole (B95; 300 mg) as a solid. LCMS (ES, m/z): 364 [M+H]⁺.

### Synthesis of Intermediate B96

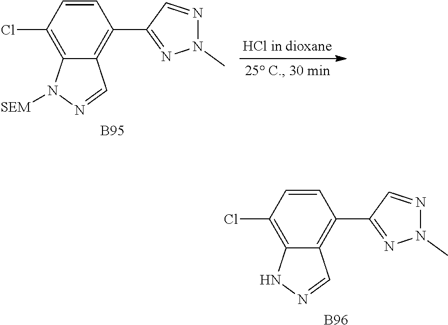

A solution of 7-chloro-4-(2-methyl-1,2,3-triazol-4-yl)-1-[[2-(trimethylsilyl)ethoxy]methyl]indazole (B95; 300 mg, 0.82 mmol) in a mixture of HCl in 1,4-dioxane (5 mL) was stirred for 30 min at 25° C. The resulting mixture was concentrated under vacuum, and purified by reverse flash chromatography on a C18 column eluting with acetonitrile in aqueous ammonium carbonate (10 mmol/L) (20% acetonitrile for 5 min, then increased to 50% over 20 min), to afford 7-chloro-4-(2-methyl-1,2,3-triazol-4-yl)-1H-indazole (B96; 80 mg) as a solid. LCMS (ES, m/z): 234 [M+H]⁺.

### Synthesis of Intermediate B97

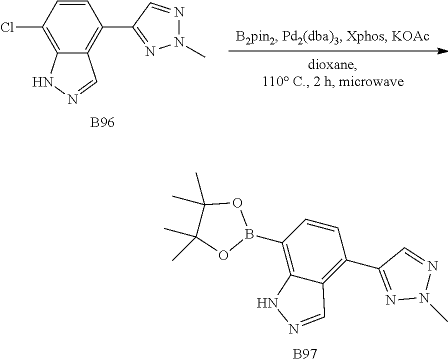

A solution of 7-chloro-4-(2-methyl-1,2,3-triazol-4-yl)-1H-indazole (B96; 80 mg, 0.34 mmol), bis(pinacolato)diboron (128 mg, 0.5 mmol), Pd₂(dba)₃ (15 mg, 0.02 mmol), XPhos (16 mg, 0.03 mmol) and potassium acetate (66 mg, 0.672 mmol) in 1,4-dioxane (5 mL) was stirred for 2 h at 110° C. under a nitrogen atmosphere. The resulting mixture was then concentrated under reduced pressure and purified by silica gel column chromatography, eluting with petroleum ether/ethyl acetate (1:1) to afford 4-(2-methyl-1,2,3-triazol-4-yl)-7-(4,4,5,5-tetramethyl-1,3,2-dioxaborolan-2-yl)-1H-indazole (B97; 80 mg) as a solid. LCMS (ES, m/z): 326 [M+H]⁺.

### Synthesis of Intermediate B98

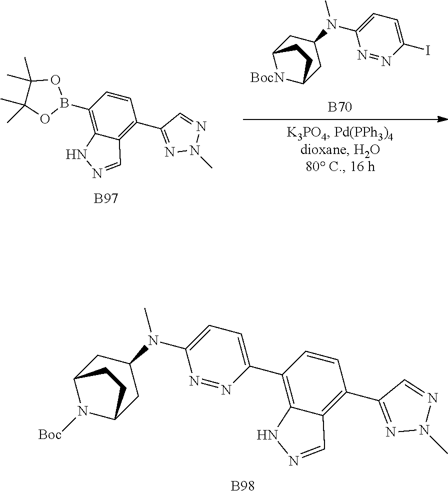

A solution of 4-(2-methyl-1,2,3-triazol-4-yl)-7-(4,4,5,5-tetramethyl-1,3,2-dioxaborolan-2-yl)-1H-indazole (B97; 80 mg, 0.24 mmol), tert-butyl (1R,3S,5S)-3-[(6-iodopyridazin-3-yl)(methyl)amino]-8-azabicyclo[3.2.1]octane-8-carboxylate (B70; 129 mg, 0.29 mmol), Pd(PPh₃)₄ (28 mg, 0.02 mmol) and K₃PO₄ (153 mg, 0.72 mmol) in 1,4-dioxane (7 mL) and H₂O (1 mL) was stirred for 16 h at 80° C. under a nitrogen atmosphere. The resulting mixture was then diluted with water (50 mL), extracted with ethyl acetate (2×50 mL), dried over anhydrous sodium sulfate, filtered, and concentrated under reduced pressure. The residue was purified by silica gel column chromatography, eluting with petroleum ether/ethyl acetate (1:3) to afford tert-butyl (1R,3S,5S)-3-[methyl([6-[4-(2-methyl-1,2,3-triazol-4-yl)-1H-indazol-7-yl]pyridazin-3-yl])amino]-8-azabicyclo[3.2.1]octane-8-carboxylate (B98; 60 mg) as a solid. LCMS (ES, m/z): 516 [M+H]⁺.

### Synthesis of Compound 144

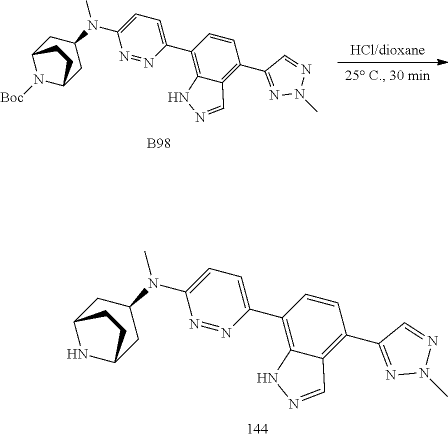

A solution of tert-butyl (1R,3S,5S)-3-[methyl([6-[4-(2-methyl-1,2,3-triazol-4-yl)-1H-indazol-7-yl]pyridazin-3-yl])amino]-8-azabicyclo[3.2.1]octane-8-carboxylate (B98; 60 mg, 0.114 mmol) in a mixture of HCl in 1,4-dioxane (3 mL) was stirred for 30 min at 25° C. The resulting mixture was concentrated under reduced pressure and purified by preparative HPLC (Condition 3), to afford (1R,3S,5S)—N-methyl-N-[6-[4-(2-methyl-1,2,3-triazol-4-yl)-1H-indazol-7-yl]pyridazin-3-yl]-8-azabicyclo[3.2.1]octan-3-amine (Compound 144; 26.3 mg) as a solid. LCMS (ES, m/z): 416 [M+H]⁺. 1H NMR (400 MHz, DMSO-d₆) δ 13.21 (s, 1H), 8.66 (s, 1H), 8.51 (s, 1H), 8.19 (d, J=9.8 Hz, 1H), 7.98 (d, J=7.7 Hz, 1H), 7.75 (d, J=7.6 Hz, 1H), 7.26 (d, J=9.8 Hz, 1H), 5.08 (s, 1H), 4.31 (s, 3H), 3.53 (s, 2H), 2.98 (s, 3H), 1.89-1.79 (m, 2H), 1.77 (d, J=2.5 Hz, 4H), 1.57 (ddd, J=12.6, 5.8, 2.6 Hz, 2H).

### Example 34: Synthesis of Compound 165

### Synthesis of Intermediate B99

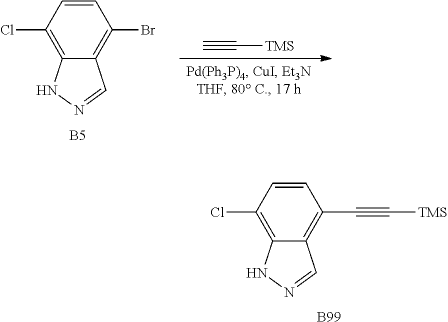

A mixture of 4-bromo-7-chloro-1H-indazole (B5; 5 g, 21.6 mmol), trimethylsilyl acetylene (2.55 g, 0.026 mmol), Pd(PPh₃)₄ (2.5 g, 2.16 mmol), copper iodide (411 mg, 2.16 mmol) and triethylamine (2.19 g, 21.6 mmol) in tetrahydrofuran (50 mL) at 20° C. was stirred for 17 h at 80° C. under a nitrogen atmosphere. The resulting mixture was then concentrated under reduced pressure, diluted with ethyl acetate (50 mL), washed with sat. sodium chloride (3×30 mL), and concentrated under reduced pressure. The residue was purified by silica gel column chromatography, eluting with petroleum ether/ethyl acetate (5:1), to afford 7-chloro-4-[2-(trimethylsilyl)ethynyl]-1H-indazole (B99; 4 g) as a solid. LCMS (ES, m/z):249 [M+H]⁺.

### Synthesis of Intermediate B100

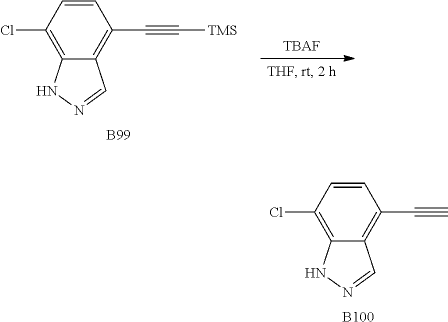

A solution of 7-chloro-4-[2-(trimethylsilyl)ethynyl]-1H-indazole (B99; 3.5 g, 0.014 mmol) and tetrabutylammonium fluoride (3.68 g, 0.014 mmol) in tetrahydrofuran (14 mL) was stirred for 2 h at room temperature. The resulting mixture was then concentrated under reduced pressure, diluted with ethyl acetate (50 mL), washed with sat. sodium chloride (3×10 mL), and concentrated under reduced pressure. The residue was purified by silica gel column chromatography, eluting with petroleum ether/ethyl acetate (5:1), to afford 7-chloro-4-ethynyl-1H-indazole (B100; 1.09 g) as a solid. LCMS (ES, m/z):177 [M+H]⁺.

### Synthesis of Intermediate B01

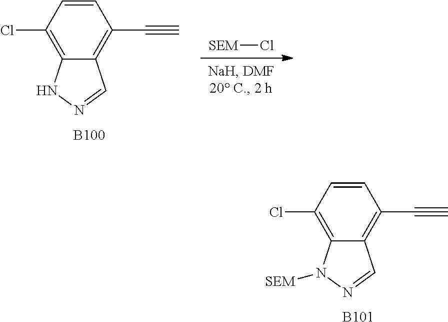

Sodium hydride (0.22 g, 9.3 mmol) was added to a solution of 7-chloro-4-ethynyl-1H-indazole (B100; 1.09 g, 6.17 mmol) in dimethylformamide (10 mL) at 0° C., followed by [2-(chloromethoxy)ethyl]trimethylsilane (2.06 g, 12.3 mmol) dropwise over 5 min at 0° C. The resulting mixture was stirred for an additional 2 h at 20° C., and was then quenched with ice water (10 mL). The resulting mixture was diluted with ethyl acetate (30 mL), washed with sat. sodium chloride (3×10 mL), and concentrated under reduced pressure. The residue was purified by silica gel column chromatography, eluting with petroleum ether/ethyl acetate (5:1) to afford 7-chloro-4-ethynyl-1-[[2-(trimethylsilyl)ethoxy]methyl]indazole (B101; 1.2 g) as an oil. LCMS (ES, m/z):307 [M+H]⁺.

### Synthesis of Intermediate B102

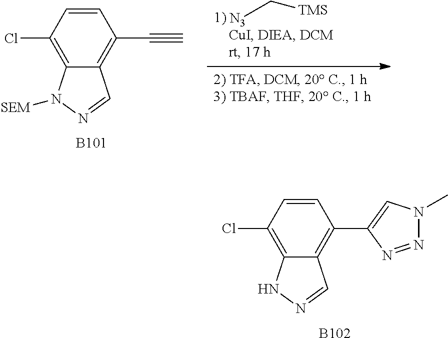

A mixture of 7-chloro-4-ethynyl-1-[[2-(trimethylsilyl)ethoxy]methyl]indazole (B101; 1 g, 3.4 mmol), (azidomethyl)trimethylsilane (2.21 g, 0.017 mmol), copper iodide (0.07 g, 0.34 mmol) and diisopropylethylamine (0.44 g, 3.42 mmol) in dichloromethane (10 mL) was stirred for 17 h at room temperature under an argon atmosphere. The resulting mixture was then diluted with ethyl acetate (30 mL), washed with sat. sodium chloride (3×10 mL), and the organic layer was concentrated under vacuum. The residue was then added to dichloromethane (10 mL) and trifluoroacetic acid (585 mg, 5.1 mmol) and stirred for 1h at 20° C., then concentrated under vacuum. Next, the residue was added to a solution of tetrabutylammonium fluoride (1.34 g, 5.13 mmol) in tetrahydrofuran (10 mL) and stirred for 1h at 20° C., then concentrated under vacuum. The residue was diluted with ethyl acetate (30 mL) and washed with sat. sodium chloride (3×10 mL). The combined organic layers were concentrated under vacuum, and the residue was purified by silica gel column chromatography, eluting with petroleum ether/ethyl acetate (5:1), to afford 7-chloro-4-(1-methyl-1,2,3-triazol-4-yl)-1H-indazole (B102; 300 mg) as a solid. LCMS (ES, m/z):234 [M+H]⁺.

### Synthesis of Intermediate B103

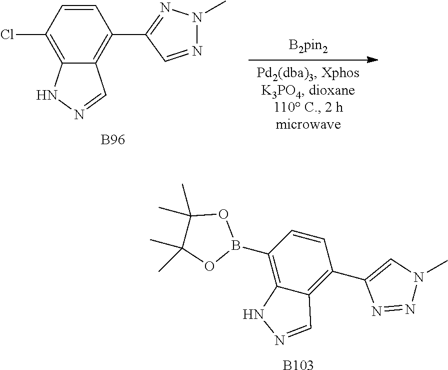

A mixture of 7-chloro-4-(1-methyl-1,2,3-triazol-4-yl)-1H-indazole (B102; 270 mg, 1.16 mmol), B₂pin₂ (587 mg, 2.31 mmol), Pd₂(dba)₃ (52.9 mg, 0.06 mmol), X-Phos (55 mg, 0.12 mmol) and K₃PO₄ (736 mg, 3.47 mmol) in dioxane (15 mL) was irradiated with microwave for 2 h at 110° C. The resulting mixture was then concentrated under vacuum and purified by silica gel column chromatography, eluting with petroleum ether/ethyl acetate (1:1) to afford 4-(1-methyl-1,2,3-triazol-4-yl)-7-(4,4,5,5-tetramethyl-1,3,2-dioxaborolan-2-yl)-1H-indazole (B103; 90 mg) as a solid. LCMS (ES, m/z):325 [M+H]⁺.

### Synthesis of Intermediate B104

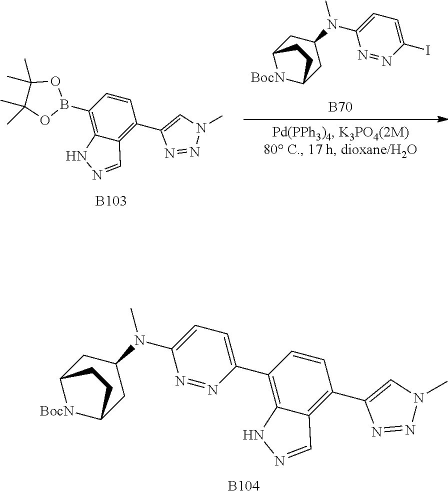

A mixture of 4-(1-methyl-1,2,3-triazol-4-yl)-7-(4,4,5,5-tetramethyl-1,3,2-dioxaborolan-2-yl)-1H-indazole (B103; 90 mg, 0.28 mmol), tert-butyl (1R,3S,5S)-3-[(6-iodopyridazin-3-yl)(methyl)amino]-8-azabicyclo[3.2.1]octane-8-carboxylate (B70; 123 mg, 0.28 mmol), Pd(PPh₃)₄ (32 mg, 0.03 mmol) and K₃PO₄ (176 mg, 0.83 mmol) in dioxane (8 mL) and H₂O (1.6 mL) was stirred for 17 h at 80° C. under an argon atmosphere. The resulting mixture was concentrated under reduced pressure, diluted with ethyl acetate (20 mL), and washed with sat. sodium chloride (3×5 mL). The resulting mixture was concentrated under reduced pressure, and purified by silica gel column chromatography, eluting with petroleum ether/ethyl acetate (1:1), to afford tert-butyl (1R,3S,5S)-3-[methyl ([6-[4-(1-methyl-1,2,3-triazol-4-yl)-1H-indazol-7-yl]pyridazin-3-yl])amino]-8-azabicyclo[3.2.1]octane-8-carboxylate (B104; 45 mg) as a solid. LCMS (ES, m/z):516 [M+H]⁺.

### Synthesis of Compound 165

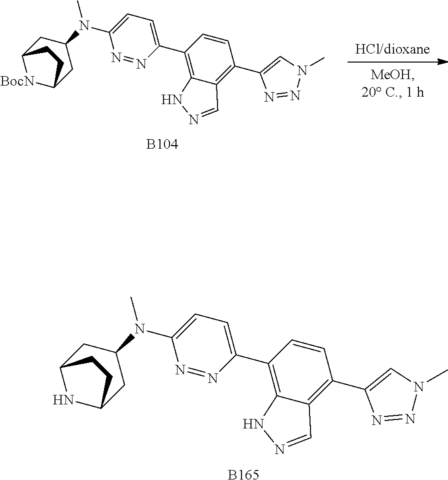

A mixture of tert-butyl (1R,3S,5S)-3-[methyl([6-[4-(1-methyl-1,2,3-triazol-4-yl)-1H-indazol-7-yl]pyridazin-3-yl])amino]-8-azabicyclo[3.2.1]octane-8-carboxylate (45 mg, 0.09 mmol), HCl in 1,4-dioxane (2 mL, 35 mmol), and methanol (2 mL) was stirred for 1h at 20° C. The resulting mixture was concentrated under reduced pressure, and purified by preparative HPLC (Condition 4, Gradient 3), to afford (1R,3S,5S)—N-methyl-N-[6-[4-(1-methyl-1,2,3-triazol-4-yl)-1H-indazol-7-yl]pyridazin-3-yl]-8-azabicyclo[3.2.1]octan-3-amine (Compound 165; 14.3 mg). LCMS (ES, m z):416 [M+H]⁺. 1H NMR (400 MHz, DMSO-d₆, ppm) δ 13.19 (s, 1H), 8.85 (s, 1H), 8.68 (s, 1H), 8.18 (d, J=9.8 Hz, 1H), 7.98 (d, J=7.7 Hz, 1H), 7.74 (d, J=7.6 Hz, 1H), 7.27 (d, J=9.8 Hz, 1H), 5.09 (s, 1H), 4.18 (s, 3H), 3.58 (s, 2H), 2.98 (s, 3H), 1.88 (td, J=12.1, 3.0 Hz, 2H), 1.80 (s, 4H), 1.60 (dd, J=13.3, 6.0 Hz, 2H).

### Example 35: Synthesis of Compound 166

### Synthesis of Intermediate B105

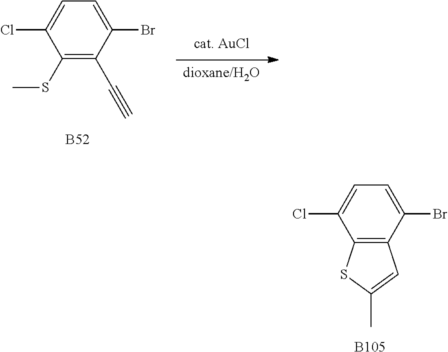

A mixture of 1-bromo-4-chloro-2-ethynyl-3-(methylsulfanyl) benzene (B52 from Example 23; 640 mg, 2.45 mmol) and gold chloride (57 mg, 0.25 mmol) in dioxane/water (4:1; 8 mL) was stirred for 6 h at 25° C. under an atmosphere of nitrogen. The reaction was then quenched by the addition of water (20 mL), and the resulting solution was extracted with ethyl acetate (3×20 mL) dried over anhydrous sodium sulfate, filtered, and concentrated under vacuum, to afford 4-bromo-7-chloro-2-methyl-1-benzothiophene (B105; 780 mg) as a solid.

### Synthesis of Intermediate B106

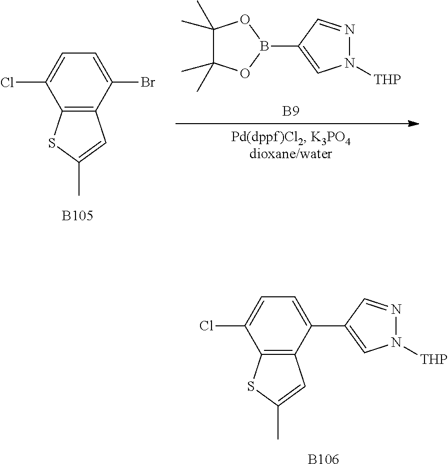

A mixture of 4-bromo-7-chloro-2-methyl-1-benzothiophene (B105; 750 mg, 2.87 mmol), 1-(oxan-2-yl)-4-(4,4,5,5-tetramethyl-1,3,2-dioxaborolan-2-yl) pyrazole (B9; 957 mg, 3.44 mmol), K₃PO₄ (1.22 g, 5.73 mmol), and Pd(dppf)Cl₂—CH₂Cl₂ (117 mg, 0.14 mmol) in dioxane/water (4:1; 15 mL) was stirred for 4 h at 100° C. The reaction was then quenched with water (20 mL) and extracted with ethyl acetate (3×20 mL). The combined organic layers were dried over anhydrous sodium sulfate, filtered, and concentrated under vacuum. The residue was purified by silica gel column chromatography eluting with ethyl acetate/petroleum ether (1:5), to afford 4-(7-chloro-2-methyl-1-benzothiophen-4-yl)-1-(oxan-2-yl) pyrazole (B106; 690 mg) as an oil. LCMS (ES, m/z): 333 [M+H]⁺.

### Synthesis of Intermediate B107

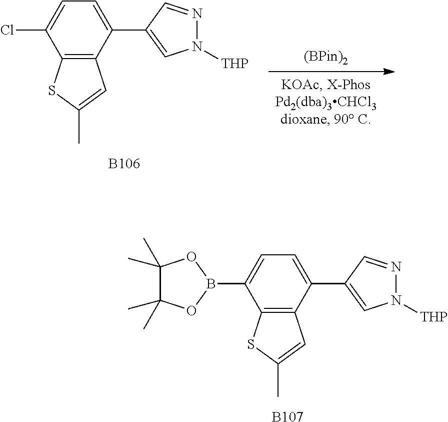

A mixture of 4-(7-chloro-2-methyl-1-benzothiophen-4-yl)-1-(oxan-2-yl) pyrazole (B106; 220 mg, 0.66 mmol), bis(pinacolato)diboron (336 mg, 1.32 mmol), potassium acetate (195 mg, 1.98 mmol), X-Phos (63 mg, 0.13 mmol), and Pd₂(dba)₃. CHCl₃ (68.4 mg, 0.066 mmol) in dioxane (5 mL) was stirred for 1.5 h at 90° C. The solids were then filtered, and the filtrate was used directly in the next step. LCMS (ES, m/z): 425 [M+H]⁺.

### Synthesis of Intermediate B108

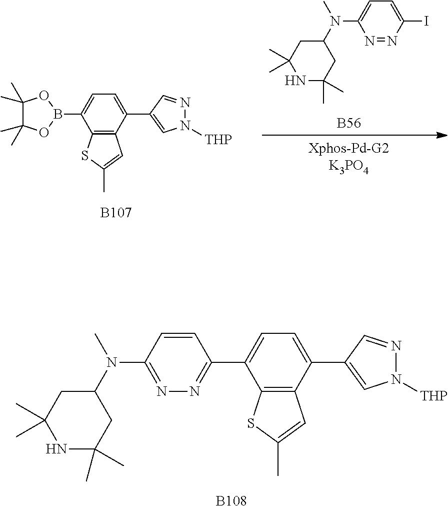

A mixture of 4-[2-methyl-7-(4,4,5-trimethyl-1,3,2-dioxaborolan-2-yl)-1-benzothiophen-4-yl]-1-(oxan-2-yl) pyrazole (B107; 194 mg, 0.47 mmol), 6-iodo-N-Methyl-N-(2,2,6,6-tetramethylpiperidin-4-yl) pyridazin-3-amine (B56; 265 mg, 0.71 mmol), K₃PO₄ (301 mg, 1.4 mmol), and XPhos Pd G2 (18.6 mg, 0.024 mmol), in dioxane/water (4:1; 80 mL) was stirred for 4 h at 100° C. under an atmosphere of nitrogen. The reaction was then quenched by the addition of water (of 15 mL), and the resulting solution was extracted with ethyl acetate (3×15 mL). The combined organic layers were dried over anhydrous sodium sulfate, filtered, and concentrated under vacuum. The residue was purified by silica gel column chromatography eluting with dichloromethane/methanol (10:1), to afford N-methyl-6-[2-methyl-4-[1-(oxan-2-yl) pyrazol-4-yl]-1-benzothiophen-7-yl]-N-(2,2,6,6-tetramethylpiperidin-4-yl) pyridazin-3-amine (B108; 220 mg) as a solid. LCMS (ES, m/z): 545 [M+H]⁺.

### Synthesis of Compound 166

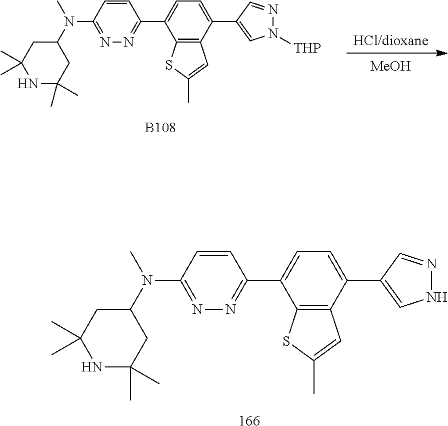

A mixture of N-methyl-6-[2-methyl-4-[1-(oxan-2-yl) pyrazol-4-yl]-1-benzothiophen-7-yl]-N-(2,2,6,6-tetramethylpiperidin-4-yl) pyridazin-3-amine (B108; 210 mg, 0.39 mmol), HCl in 1,4-dioxane (1 mL, 17.5 mmol), and methanol (4 mL) was stirred for 2 h at 25° C., then concentrated under vacuum. The residue was then dissolved in methanol (3 mL) and filtered. The crude product was purified by preparative HPLC (Condition 4, Gradient 1), to afford methyl-6-[2-methyl-4-(1H-pyrazol-4-yl)-1-benzothiophen-7-yl]-N-(2,2,6,6-tetramethylpiperidin-4-yl) pyridazin-3-amine (Compound 166; 26.7 mg) as a solid. LCMS (ES, m/z): 461 [M+H]⁺. ¹H NMR (400 MHz, Methanol-d₄, ppm) δ 8.04 (d, J=9.7 Hz, 1H), 7.75 (d, J=7.6 Hz, 1H), 7.72 (s, 2H), 7.35 (d, J=7.6 Hz, 1H), 7.32-7.24 (m, 2H), 5.22 (s, 1H), 3.07 (s, 3H), 2.07 (d, J=1.1 Hz, 3H), 1.77 (dd, J=12.6, 3.6 Hz, 2H), 1.65 (t, J=12.5 Hz, 2H), 1.45 (s, 6H), 1.29 (s, 6H), 0.12 (s, 1H).

### Example 36: Synthesis of Compound 167

### Synthesis of Intermediate B110

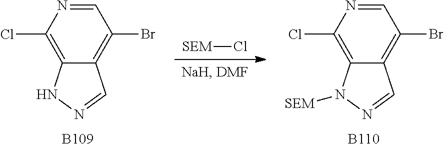

Sodium hydride (253 mg, 6.32 mmol, 60%) was added in portions to a solution of 4-bromo-7-chloro-1H-pyrazolo[3,4-c]pyridine (B109; 1 g, 4.22 mmol) in dimethylformamide (10 mL) at 0° C., and the mixture was stirred for 30 min at 25° C. [2-(Chloromethoxy)ethyl]trimethylsilane (843 mg, 5 mmol) was then added dropwise over 5 min at 0° C., and the resulting mixture was stirred for additional 1 h at 25° C. The mixture was then poured into water (100 mL) and extracted with ethyl acetate (2×100 mL). The combined organic layers were washed with brine (2×100 mL), dried over anhydrous sodium sulfate, filtered, and concentrated under reduced pressure. The residue was purified by silica gel column chromatography, eluting with petroleum ether/ethyl acetate (10:1), to afford 4-bromo-7-chloro-1-[[2-(trimethylsilyl)ethoxy]methyl]pyrazolo[3,4-c]pyridine (B110; 1.2 g) as an oil. LCMS (ES, m/z): 363/365 [M+H]⁺.

### Synthesis of Intermediate B111

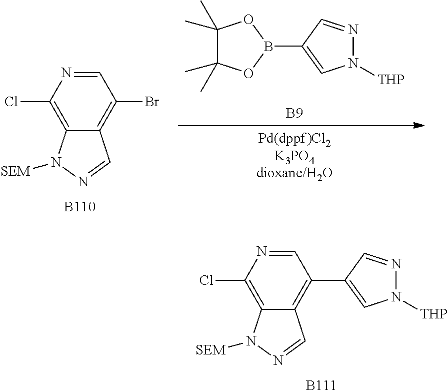

A mixture of 4-bromo-7-chloro-1-[[2-(trimethylsilyl)ethoxy]methyl]pyrazolo[3,4-c]pyridine (B110; 1.2 g, 3.24 mmol), 1-(oxan-2-yl)-4-(4,4,5,5-tetramethyl-1,3,2-dioxaborolan-2-yl)pyrazole (B9; 902 mg, 3.24 mmol), Pd(dppf)Cl₂.CH₂Cl₂ (264 mg, 0.32 mmol) and K₃PO₄ (2.06 g, 9.7 mmol) in 1,4-dioxane (18 mL) and H₂O (6 mL) was stirred for 16 h at 80° C. under a nitrogen atmosphere. The resulting mixture was then poured onto water (150 mL), extracted with ethyl acetate (3×150 mL), dried over anhydrous sodium sulfate, filtered, and concentrated under reduced pressure. The residue was purified by silica gel column chromatography, eluting with petroleum ether/ethyl acetate (5:1), to afford 4-(7-chloro-1-[[2-(trimethylsilyl)ethoxy]methyl]pyrazolo[3,4-c]pyridin-4-yl)-1-(oxan-2-yl)pyrazole (B111; 630 mg) as a solid. LCMS (ES, m/z): 434 [M+H]⁺.

### Synthesis of Intermediate B112

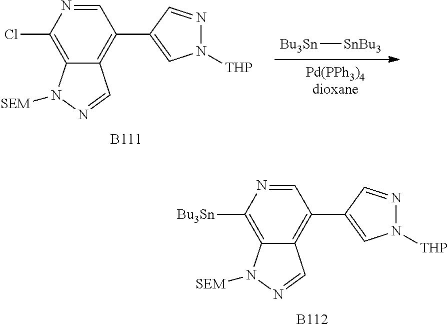

A solution of 4-(7-chloro-1-[[2-(trimethylsilyl)ethoxy]methyl]pyrazolo[3,4-c]pyridin-4-yl)-1-(oxan-2-yl)pyrazole (B111; 630 mg, 1.42 mmol), hexabutyldistannane (1.65 g, 2.84 mmol) and Pd(PPh₃)₄ (164 mg, 0.142 mmol) in 1,4-dioxane (10 mL) was stirred overnight at 100° C. under a nitrogen atmosphere. The resulting mixture was concentrated under vacuum, and purified by silica gel column chromatography, eluting with petroleum ether/ethyl acetate (1:1), to afford 1-(oxan-2-yl)-4-[7-(tributylstannyl)-1-[[2-(trimethylsilyl)ethoxy]methyl]pyrazolo[3,4-c]pyridin-4-yl]pyrazole (B112; 100 mg) as an oil.

### Synthesis of Intermediate B113

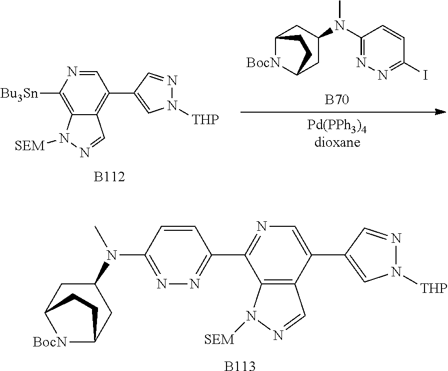

A solution of 1-(oxan-2-yl)-4-[7-(tributylstannyl)-1-[[2-(trimethylsilyl)ethoxy]methyl]pyrazolo[3,4-c]pyridin-4-yl]pyrazole (B112; 100 mg, 0.15 mmol), tert-butyl (1R,3S,5S)-3-[(6-iodopyridazin-3-yl)(methyl)amino]-8-azabicyclo[3.2.1]octane-8-carboxylate (B70; 65 mg, 0.15 mmol) and Pd(PPh₃)₄ (17 mg, 0.015 mmol) in 1,4-dioxane (3 mL) was stirred for 16 h at 100° C. under a nitrogen atmosphere. The resulting mixture was concentrated under vacuum, and purified by silica gel column chromatography, eluting with petroleum ether/ethyl acetate (5:1) to afford tert-butyl (1R,3S,5S)-3-[methyl(6-[4-[1-(oxan-2-yl)pyrazol-4-yl]-1-[[2-(trimethylsilyl)ethoxy]methyl]pyrazolo[3,4-c]pyridin-7-yl]pyridazin-3-yl)amino]-8-azabicyclo[3.2.1]octane-8-carboxylate (B113; 40 mg) as a solid. LCMS (ES, m/z): 716 [M+H]⁺.

### Synthesis of Compound 167

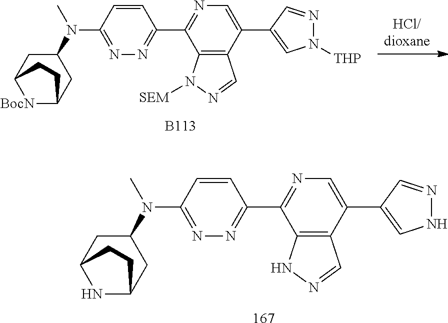

A solution of tert-butyl (1R,3S,5S)-3-[methyl(6-[4-[1-(oxan-2-yl)pyrazol-4-yl]-1-[[2-(trimethylsilyl)ethoxy]methyl]pyrazolo[3,4-c]pyridin-7-yl]pyridazin-3-yl)amino]-8-azabicyclo[3.2.1]octane-8-carboxylate (B113; 40 mg, 0.022 mmol), in HCl in 1,4-dioxane (1 mL), was stirred for 30 min at 25° C. The resulting mixture was then concentrated under reduced pressure, and purified by preparative HPLC (Condition 6, Gradient 1), to afford (1R,3S,5S)—N-methyl-N-[6-[4-(1H-pyrazol-4-yl)-1H-pyrazolo[3,4-c]pyridin-7-yl]pyridazin-3-yl]-8-azabicyclo[3.2.1]octan-3-amine (Compound 167; 4.9 mg) as a solid. LCMS (ES, m/z): 402 [M+H]⁺. 1H NMR (400 MHz, DMSO-d₆) δ 8.80 (s, 1H), 8.66 (s, 1H), 8.60 (d, J=9.7 Hz, 1H), 8.50 (s, 2H), 7.71 (d, J=9.9 Hz, 1H), 5.07 (s, 1H), 4.14 (s, 2H), 3.12 (s, 3H), 2.26 (t, J=12.5 Hz, 2H), 2.08 (s, 4H), 1.88 (d, J=12.7 Hz, 2H).

### Example 37: Synthesis of Compound 147

### Synthesis of Intermediate B115

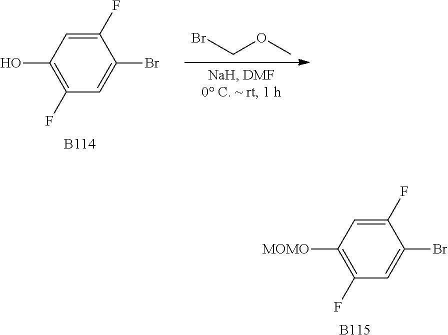

To a stirred solution of 4-bromo-2,5-difluorophenol (B114, 9.00 g, 42.203 mmol) in DMF (90 mL) was added NaH (2.53 g, 63.305 mmol, 60%) in portions at 0° C. under nitrogen. The resulting mixture was stirred for 30 min at 0° C., then bromomethoxy-methane (6.33 g, 50.644 mmol) was added dropwise at 0° C. The resulting mixture was stirred for an additional 1 h at room temperature, then the resulting mixture was poured into water/ice (500 mL). The mixture was extracted with EA (2×500 mL), and the combined organic layers were washed with brine (3×500 mL), dried over anhydrous Na₂SO₄, then the filtrate was concentrated under reduced pressure. The residue was purified by silica gel column chromatography, eluted with PE/EA (10:1) to afford 1-bromo-2,5-difluoro-4-(methoxymethoxy)benzene (B115, 10 g, 91.77%) as a colorless oil. LCMS (ES, m/z): 253/255 [M+H]⁺

### Synthesis of Intermediate B116

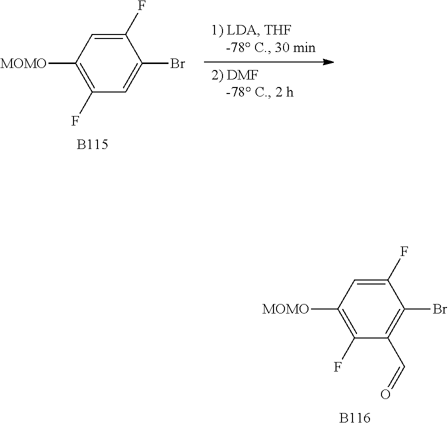

To a stirred solution of 1-bromo-2,5-difluoro-4-(methoxymethoxy)benzene (B115, 10.00 g, 38.729 mmol) in THE (100 mL) was added LDA (25.00 mL, 50.347 mmol) dropwise at −78° C. under nitrogen. The resulting mixture was stirred for 30 min at −78° C., then DMF was added (3.11 g, 42.548 mmol) dropwise over 15 min at −78° C. The resulting mixture was stirred for additional 2 h at −78° C., then poured into saturated sodium bicarbonate aqueous solution (500 mL) and extracted with EA (3×500 mL). The combined organic layers were washed with brine (2×1 L), dried over anhydrous Na₂SO₄, and the filtrate was concentrated under reduced pressure. The residue was purified by silica gel column chromatography, eluted with PE/EtOAc (10:1) to afford 2-bromo-3,6-difluoro-5-(methoxymethoxy)benzaldehyde (7 g, 63.02%) a solid. LCMS (ES, m/z): 281/283 [M+H]⁺

### Synthesis of Intermediate B118

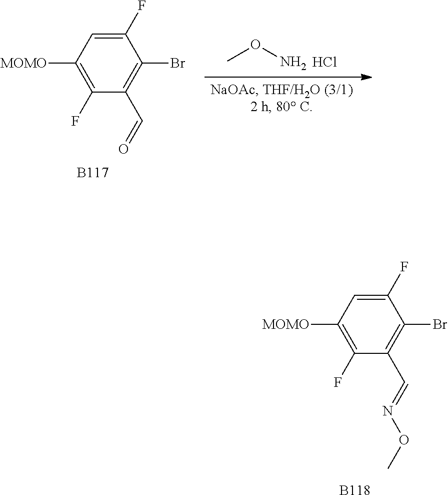

To a stirred solution of O-methylhydroxylamine hydrochloride (2.21 g, 26.462 mmol) and NaOAc (2.37 g, 28.891 mmol) in THE (69 mL)) and H₂O (23 mL) was added 2-bromo-3,6-difluoro-5-(methoxymethoxy)benzaldehyde (6.90 g, 24.060 mmol) dropwise at room temperature. The resulting mixture was stirred for 2 h at 80 degrees C. The mixture was allowed to cool down to 25 degrees C., and the aqueous layer was extracted with EA (2×200 mL). The resulting mixture was concentrated under reduced pressure to afford (E)-[[2-bromo-3,6-difluoro-5-(methoxymethoxy)phenyl]methylidene](methoxy)amine (7 g, 91.95%) as a solid. LCMS (ES, m z): 310/312 [M+H]⁺

### Synthesis of Intermediate B120

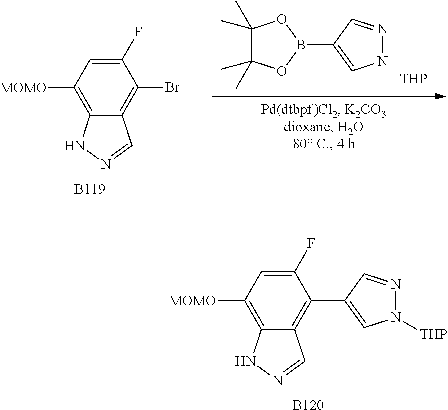

A solution of 4-bromo-5-fluoro-7-(methoxymethoxy)-1H-indazole (2.00 g, 7.125 mmol), 1-(oxan-2-yl)-4-(4,4,5,5-tetramethyl-1,3,2-dioxaborolan-2-yl)pyrazole (2.18 g, 7.837 mmol), Pd(DtBPF)Cl₂ (464 mg, 0.713 mmol) and K₂CO₃ (2M)(10.5 mL, 21.345 mmol) in 1,4-dioxane (20 mL) and H₂O (5 mL) was stirred for 4 h at 80 degrees C. under nitrogen atmosphere. The mixture was allowed to cool down to room temperature. The resulting mixture was poured into water (100 mL), then extracted with EA (2×100 mL), dried over anhydrous Na₂SO₄, filtered, and then concentrated under reduced pressure. The residue was purified by silica gel column chromatography, eluted with PE/EA (5:1) to afford 5-fluoro-7-(methoxymethoxy)-4-[1-(oxan-2-yl)pyrazol-4-yl]-1H-indazole (2 g, 79.42%) as a solid. LCMS (ES, m/z): 347 [M+H]⁺

### Synthesis of Intermediate B121

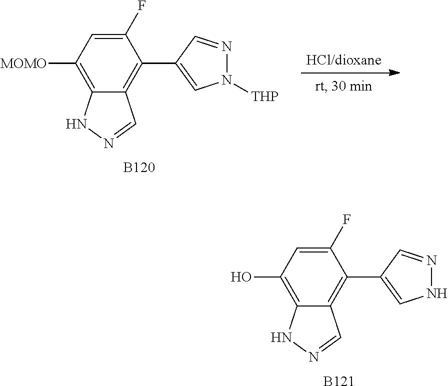

A solution of 5-fluoro-7-(methoxymethoxy)-4-[1-(oxan-2-yl)pyrazol-4-yl]-1H-indazole (1.80 g, 5.093 mmol) in HCl(gas) in 1,4-dioxane (20 mL) was stirred for 30 min at room temperature. The resulting mixture was concentrated under reduced pressure, and the residue was purified by reverse flash chromatography (Condition 4, Gradient 3) to afford 5-fluoro-4-(1H-pyrazol-4-yl)-1H-indazol-7-ol (600 mg, 52.91%) as a solid. LCMS (ES, m/z): 219 [M+H]⁺

### Synthesis of Intermediate B122

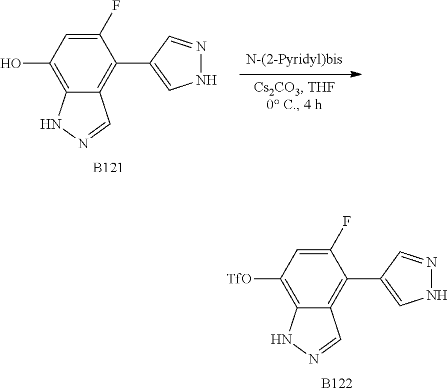

To a stirred mixture of 5-fluoro-4-(1H-pyrazol-4-yl)-1H-indazol-7-ol (350 mg, 1.572 mmol) and Cs₂CO₃ (512 mg, 1.572 mmol) in THE (3 mL) was added 1,1,1-trifluoro-N-(pyridin-2-yl)-N-trifluoromethanesulfonylmethanesulfonamide (563 mg, 1.572 mmol) in THF (2 mL) dropwise at 0 degrees C. The resulting mixture was stirred for 4 h at 0 degrees C., then poured into water (50 mL), extracted with EA (2×50 mL), and dried over anhydrous Na₂SO₄. After filtration, the filtrate was concentrated under reduced pressure and the residue was purified by silica gel column chromatography, eluted with PE/EA (1:1) to afford 5-fluoro-4-(1H-pyrazol-4-yl)-1H-indazol-7-yl trifluoromethanesulfonate (130 mg, 23.14%) as a solid. LCMS (ES, m/z): 351 [M+H]⁺

### Synthesis of Intermediate B123

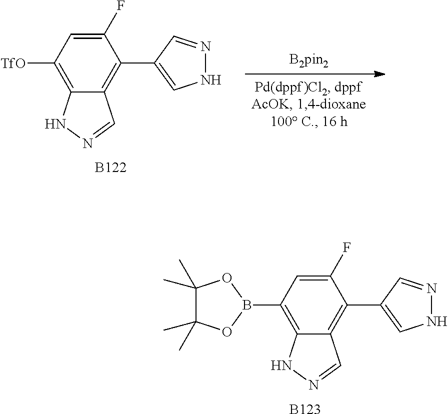 [SMILES: "B122" ; O=S(=O)(Oc1cc(F)c(-c2cn[nH]c2)c2cn[nH]c12)C(F)(F)F ; "B123" ; CC1(C)OB(c2cc(F)c(-c3cn[nH]c3)c3cn[nH]c23)OC1(C)C ; "B2pin2" ; "Pd(dppf)Cl2, dppfAcOK, 1,4-dioxane100° C., 16 h"]

A solution of 5-fluoro-4-(1H-pyrazol-4-yl)-1H-indazol-7-yl trifluoromethanesulfonate (130 mg, 0.364 mmol), bis(pinacolato)diboron (138 mg, 0.546 mmol), Pd(dppf)Cl₂ (30 mg, 0.036 mmol), Dppf (20 mg, 0.036 mmol) and AcOK (107 mg, 1.092 mmol) in 1,4-dioxane (5.00 mL) was stirred for 16 h at 100 degrees C. under nitrogen atmosphere. The mixture was allowed to cool down to room temperature, then poured into water (50 mL), extracted with EA (2×50 mL), and dried over anhydrous Na₂SO₄. After filtration, the filtrate was concentrated under reduced pressure to afford 5-fluoro-4-(1H-pyrazol-4-yl)-7-(4,4,5,5-tetramethyl-1,3,2-dioxaborolan-2-yl)-1H-indazole (70 mg, 57.47%) as a solid. LCMS (ES, m/z): 329 [M+H]⁺

### Synthesis of Intermediate B124

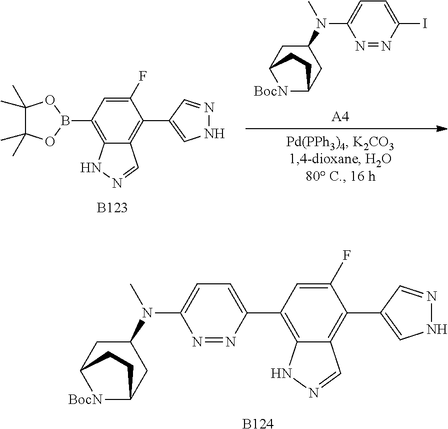

A solution of 5-fluoro-4-(1H-pyrazol-4-yl)-7-(4,4,5,5-tetramethyl-1,3,2-dioxaborolan-2-yl)-1H-indazole (70 mg, 0.209 mmol), tert-butyl (1R,3S,5S)-3-[(6-iodopyridazin-3-yl)(methyl)amino]-8-azabicyclo[3.2.1]octane-8-carboxylate (111 mg, 0.251 mmol), Pd(PPh₃)₄ (24 mg, 0.021 mmol) and K₂CO₃ (87 mg, 0.627 mmol) in 1,4-dioxane (3.00 mL) and H₂O (0.30 mL) was stirred for 16 h at 80 degrees C. under nitrogen atmosphere. The mixture was allowed to cool down to room temperature, concentrated under reduced pressure, and the residue was purified by silica gel column chromatography, eluted with PE/EA (1:2) to afford tert-butyl (1R,3S,5S)-3-([6-[5-fluoro-4-(1H-pyrazol-4-yl)-1H-indazol-7-yl]pyridazin-3-yl](methyl)amino)-8-azabicyclo[3.2.1]octane-8-carboxylate (20 mg, 18.45%) as a solid. LCMS (ES, m/z): 519 [M+H]⁺

### Synthesis of Compound 147

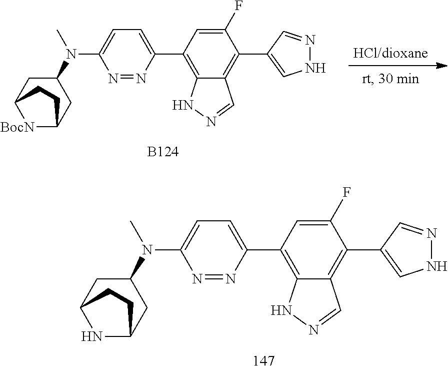

A solution of tert-butyl (1R,3S,5S)-3-([6-[5-fluoro-4-(l-pyrazol-4-yl)-1H-indazol-7-yl]pyridazin-3-yl](methyl)amino)-8-azabicyclo[3.2.1]octane-8-carboxylate (20 mg, 0.039 mmol) in HCl(gas) in 1,4-dioxane (1.00 mL) was stirred for 30 min at room temperature. The resulting mixture was concentrated under reduced pressure, and the crude product was purified by Prep-HPLC (Condition 3, Gradient 2) to afford (1R,3S,5S)—N-[6-[5-fluoro-4-(1H-pyrazol-4-yl)-1H-indazol-7-yl]pyridazin-3-yl]-N-methyl-8-azabicyclo[3.2.1]octan-3-amine (3.1 mg, 18.82%) as a solid. LCMS (ES, m/z): 419 [M+H]^{+ 1}H NMR (400 MHz, DMSO-d₆) δ 13.32 (s, 1H), 13.19 (s, 1H), 8.49 (s, 1H), 8.28 (s, 2H), 8.22 (d, J=9.8 Hz, 1H), 7.94 (d, J=12.6 Hz, 1H), 7.27 (d, J=9.8 Hz, 1H), 5.15-5.07 (m, 1H), 3.65 (s, 2H), 2.99 (s, 3H), 1.92 (td, J=12.3, 3.0 Hz, 2H), 1.83 (s, 4H), 1.67-1.57 (m, 2H).

### Example 38: Synthesis of Compounds 187, 196, 197, and 209

### Synthesis of Intermediate B126

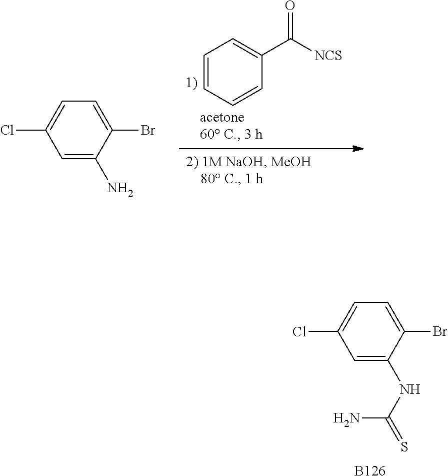

Benzoyl isothiocyanate (52.17 g, 319.659 mmol, 1.10 equiv) was added dropwise to a stirred solution of 2-bromo-5-chloroaniline (60.00 g, 290.598 mmol, 1.00 equiv) in acetone (600.00 mL). The reaction mixture was heated to reflux for 3 h. After which, the reaction mixture was poured into water-ice, and stirred for an additional 30 min to form a preciptate. The precipitate was collected by filtration and washed with water. The solid was dissolved in methanol (600 mL) and treated with 1N NaOH (150 mL). The reaction mixture was heated to 80° C. for 2 h. After cooling, the reaction mixture was poured into a water-ice mixture, and sufficient aqueous 1N HCl was added to produce a neutral (pH ˜7) solution. A precipitate formed that was collected by filtration and dried to afford 2-bromo-5-chlorophenylthiourea (70 g, 90%). LCMS (ES, m/z): 265 [M+H]⁺.

### Synthesis of Intermediate B127

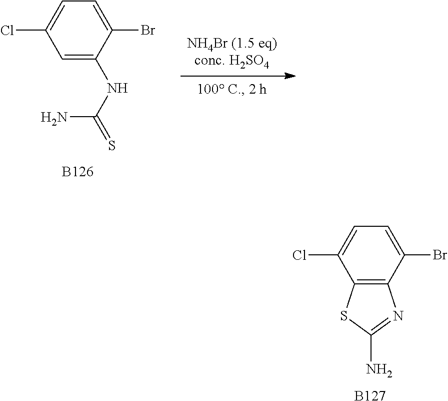

To a stirred solution of 2-bromo-5-chlorophenylthiourea (70.00 g, 263.606 mmol, 1.00 equiv) in concentrated H₂SO₄ (200 mL) was added NH₄Br (23.79 g, 263.606 mmol, 1.00 equiv) over the course of 1 h. The reaction mixture was heated at 100° C. for 2 h, then cooled to room temperature and poured into ice water (1500 mL) to afford a precipitate. The mixture was neutralized to pH-7 with aqueous ammonium hydroxide solution. The solid was collected by filtration, washed with water, and dried in vacuum to afford 4-bromo-7-chloro-1,3-benzothiazol-2-amine (65 g, 92%) as a solid. LCMS (ES, m/z): 263 [M+H]⁺.

### Synthesis of Intermediate B128

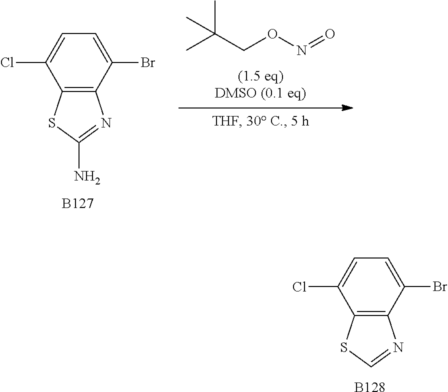

A solution of 4-bromo-7-chloro-1,3-benzothiazol-2-amine (55.00 g, 208 mmol, 1.00 equiv) in THF (300 mL, 4937 mmol, 23.66 equiv) was added dropwise over 20 min to a mixture of t-BuNO₂ (32.28 g, 313 mmol, 1.50 equiv) and DMSO (1.630 g) in THF (100 mL) at 25° C. The reaction mixture was stirred at 25° C. for 3 h, then partitioned between ethyl acetate (300 mL) and water (360 mL). The aqueous layer was extracted with ethyl acetate (2×300 mL). The organic layers were combined and washed with saturated brine (300 mL), dried over anhydrous sodium sulfate, and concentrated to give a residue. The residue was purified by silica gel column chromatography, eluted with PE/EtOAc (5:1) to afford 4-bromo-7-chloro-1,3-benzothiazole (40.4 g, 78%) as a solid. LCMS (ES, m/z): 248 [M+H]⁺.

### Synthesis of Intermediate B129

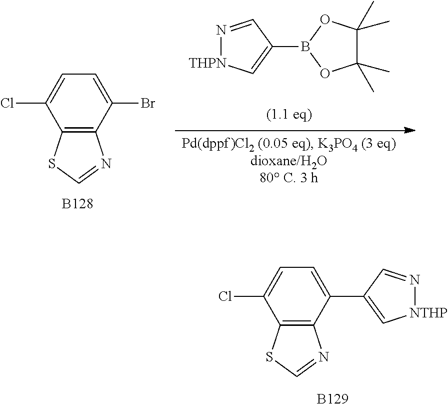

A mixture of 4-bromo-7-chloro-1,3-benzothiazole (43.00 g, 173 mmol, 1.00 equiv), 1-(oxan-2-yl)-4-(4,4,5,5-tetramethyl-1,3,2-dioxaborolan-2-yl)pyrazole (52.94 g, 190 mmol, 1.10 equiv), Pd(dppf)Cl₂. CH₂Cl₂ (7.10 g, 8.704 mmol, 0.05 equiv), K₃PO₄ (110.18 g, 519 mmol, 3.00 equiv) in H₂O (100 mL) and dioxane (400 mL) was stirred for 3h at 100° C. under a nitrogen atmosphere. The resulting mixture was concentrated under reduced pressure, then partitioned between ethyl acetate (200 mL) and water (200 mL). The aqueous layer was extracted with ethyl acetate (3×300 mL). The organic layers were combined, washed with saturated brine (300 mL), dried over anhydrous sodium sulfate, and concentrated to give a residue. The residue was purified by silica gel column chromatography, eluted with PE/EtOAc (3:1) to afford 7-chloro-4-[1-(oxan-2-yl)pyrazol-4-yl]-1,3-benzothiazole (36 g, 65%) as a solid. LCMS (ES, m/z): 320 [M+H]⁺.

### Synthesis of Intermediate B130

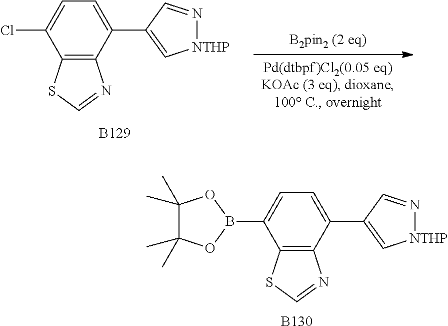

To a solution of 7-chloro-4-[1-(oxan-2-yl)pyrazol-4-yl]-1,3-benzothiazole (15.00 g, 46.9 mmol, 1.00 equiv) and B₂pin₂ (23.82 g, 0.094 mmol, 2.00 equiv) in dioxane (200 mL) was added KOAc (13.81 g, 140 mmol, 3.00 equiv) and Pd(dttbp)Cl₂ (3.820 g, 4.690 mmol, 0.10 equiv). The reaction mixture was stirred overnight at 100° C. under a nitrogen atmosphere. The resulting mixture was filtered, the filter cake was washed with ethyl acetate (3×15 mL), and the filtrate extracted with ethyl acetate (3×100 mL). The organic layers were combined and washed with saturated brine (150 mL), dried over anhydrous sodium sulfate, and concentrated to give a residue. The residue was purified by silica gel column chromatography, eluted with PE/EtOAc (3:1) to afford 4-[1-(oxan-2-yl)pyrazol-4-yl]-7-(4,4,5,5-tet ramethyl-1,3,2-d ioxaborolan-2-yl)-1,3-benzothiazole (16 g crude product 55%, purity, yield 41.47%) as an oil. LCMS (ES, m/z): 412 [M+H]⁺.

### Synthesis of Intermediate B131

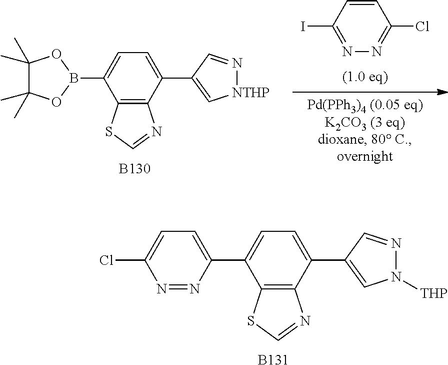

To a mixture of 4-[1-(oxan-2-yl)pyrazol-4-yl]-7-(4, 4,5,5-tetramethyl-1,3,2-dioxaborolan-2-yl)-1,3-benzothiazole (16.00 g, 21.411 mmol, 1.00 equiv, 55%), 3-chloro-6-iodopyridazine (5.138 g, 21.411 mmol, 1.00 equiv), Pd(dppf)Cl₂.CH₂Cl₂ (1747.56 mg, 2.141 mmol, 0.10 equiv), and K₂CO₃ (8877.6 mg, 64.233 mmol, 3.00 equiv) in a mixture of dioxane (100.00 mL) and H₂O (20.00 mL) was stirred overnight at 80° C. under a nitrogen atmosphere. The resulting mixture was filtered, the filter cake was washed with ethyl acetate (3×20 mL). The filtrated was diluted with ethyl acetate (100 mL) and water (100 mL), and the aqueous layer was extracted with ethyl acetate (3×100 mL). The combined organic layers were washed with brine (1×200 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10:1), to afford 7-(6-chloropyridazin-3-yl)-4-[1-(oxan-2-yl)pyrazol-4-yl]-1,3-benzothiazole (5.4 g, 59.31%) as a solid. LCMS (ES, m/z): 397 [M+H]⁺.

### Synthesis of Intermediate B132

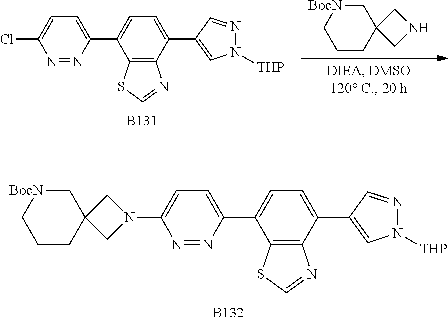

To a stirred solution of 7-(6-chloropyridazin-3-yl)-4-[1-(oxan-2-yl)pyrazol-4-yl]-1,3-benzothiazole (100 mg, 0.251 mmol, 1.00 equiv) and tert-butyl 2,6-diazaspiro[3.5]nonane-6-carboxylate (85.32 mg, 0.377 mmol, 1.5 equiv) in DMSO (10 mL) was added DIEA (97.45 mg, 0.753 mmol, 3 equiv) dropwise. The reaction mixture was stirred for 20 h at 120° C., then cooled to 25° C. The reaction mixture was quenched with water at room temperature, and extracted with ethyl acetate (3×10 mL). The combined organic layers were washed with brine (2×10 mL), dried over anhydrous Na₂SO₄, and filtered. After filtration, the filtrate was concentrated under reduced pressure to afford tert-butyl 2-(6-{4-[1-(oxan-2-yl) pyrazol-4-yl]-1,3-benzothiazol-7-yl}pyridazin-3-yl)-2,6-diazaspiro[3.5]nonane-6-carboxylate (130 mg, 66.88%) as a solid. LCMS (ES, m/z): 588 [M+H]⁺.

### Synthesis of Compound 187

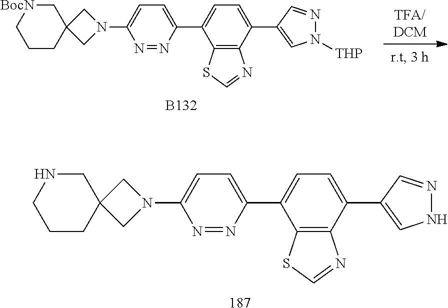

A mixture of tert-butyl 2-(6-{4-[1-(oxan-2-yl)pyrazol-4-yl]-1,3-benzothiazol-7-yl}pyridazin-3-yl)-2,6-diazaspiro[3.5]nonane-6-carboxylate and TFA (1 mL, 13.463 mmol, 60.87 equiv) in DCM (5 mL) was stirred for 3 h at 25° C. The resulting mixture was concentrated under reduced pressure to give a residue. The residue was purified by prep-HPLC (Condition 4, Gradient 7) to afford 7-(6-{2,6-diazaspiro [3.5]nonan-2-yl} pyridazin-3-yl)-4-(1H-pyrazol-4-yl)-1,3-benzothiazole (58.6 mg, 62.14%) as a solid.
Compounds 196, 197, and 209 were prepared according to the same procedure outlined in this Example 38 and generalized by Scheme B. Table 2 below provides intermediates used in these procedures and final compound characterization data.

TABLE 2
Intermediates and characterization data for compounds prepared according to Example 38 protocol and general Scheme B

| | | LCMS | |
|---|---|---|---|
| | | (ES, m/z) | ¹H NMR (400 MHz, DMSO- |
| Compound Structure | A-H | [M + H]⁺ | d₆) δ |
| 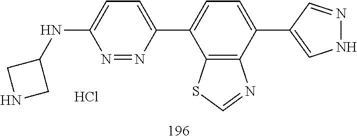 [SMILES: c1nc2c(-c3cn[nH]c3)ccc(-c3ccc(NC4CNC4)nn3)c2s1 ; Cl] | 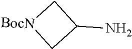 [SMILES: CC(C)(C)OC(=O)N1CC(N)C1] | 350 | 9.52 (s, 1H), 8.55 (s, 2H), 8.35 (d, J = 9.6 Hz, 1H), 8.13 (d, J = 8.1 Hz, 1H), 8.01 (d, J = 8.0 Hz, 1H), 7.19 (d, J = 9.5 Hz, 1H), 4.97 (p, J = 7.4 Hz, 1H), 4.38- 4.28 (m, 2H), 4.09-4.00 (m, 2H) |
| 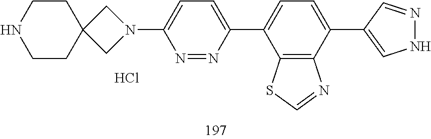 [SMILES: c1nc2c(-c3cn[nH]c3)ccc(-c3ccc(N4CC5(CCNCC5)C4)nn3)c2s1 ; Cl] | 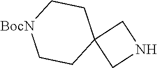 [SMILES: CC(C)(C)OC(=O)N1CCC2(CC1)CNC2] | 404 | 9.58 (s, 1H), 8.81 (s, 2H), 8.59 (s, 3H), 8.26 (d, J = 8.1 Hz, 1H), 8.06 (d, J = 8.0 Hz, 1H), 7.41 (s, 1H), 4.14 (s, 4H), 3.09 (s, 4H), 2.04 (t, J = 5.7 Hz, 4H) |
| 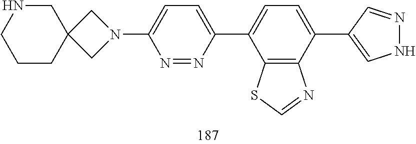 [SMILES: c1nc2c(-c3cn[nH]c3)ccc(-c3ccc(N4CC5(CCCNC5)C4)nn3)c2s1] | 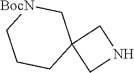 [SMILES: CC(C)(C)OC(=O)N1CCCC2(CNC2)C1] | 404 | 13.08 (s, 1H), 9.51 (s, 1H), 8.67 (s, 1H), 8.41 (s, 1H), 8.28 (d, J = 9.6 Hz, 1H), 8.12 (d, J = 8.1 Hz, 1H), 7.99 (d, J = 8.0 Hz, 1H), 6.97 (d, J = 9.5 Hz, 1H), 3.87 (d, J = 8.2 Hz, 2H), 3.79 (d, J = 8.3 Hz, 2H), 2.85 (s, 2H), 2.64 (s, 2H), 1.76 (t, J = 5.8 Hz, 2H), 1.46 (s, 2H) |
| 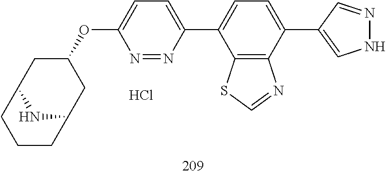 [SMILES: c1nc2c(-c3cn[nH]c3)ccc(-c3ccc(O[C@@H]4C[C@H]5CCC[C@@H](C4)N5)nn3)c2s1 ; Cl] | 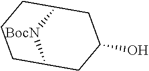 [SMILES: CC(C)(C)OC(=O)N1[C@@H]2CCC[C@H]1C[C@H](O)C2] | 419 | 9.55 (s, 1H), 9.01 (s, 1H), 8.90 (s, 1H), 8.62-8.54 (m, 3H), 8.25 (d, J = 8.1 Hz, 1H), 8.05 (d, J = 8.0 Hz, 1H), 7.43 (d, J = 9.4 Hz, 1H), 6.14 (dq, J = 11.4, 6.3, 5.6 Hz, 1H), 3.82 (s, 2H), 2.57 (s, 2H), 2.22-2.10 (m, 2H), 2.02 (d, J = 13.5 Hz, 2H), 1.93 (d, J = 13.7 Hz, 3H), 1.81 (s, 1H) |

### Example 39: Synthesis of Compound 211

### Synthesis of Intermediate B133

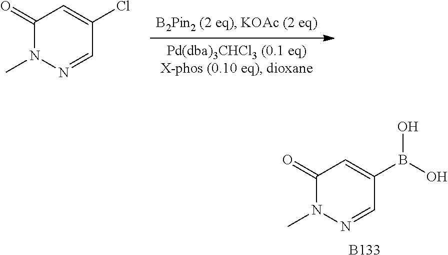

To a mixture of 5-chloro-2-methylpyridazin-3-one (100 mg, 0.69 mmol, 1.00 equiv), B₂pin₂ (351.33 mg, 1.384 mmol, 2 equiv), KOAc (135.78 mg, 1.38 mmol, 2.00 equiv), and X-Phos (32.98 mg, 0.069 mmol, 0.10 equiv) in dioxane (1.00 mL) was added Pd₂(dba)₃CHCl₃ (35.80 mg, 0.035 mmol, 0.05 equiv) under nitrogen atmosphere. The reaction mixture was stirred at 90° C. for 5 h. LCMS (ES, m/z): 155 [M+H]⁺.

### Synthesis of Compound 211

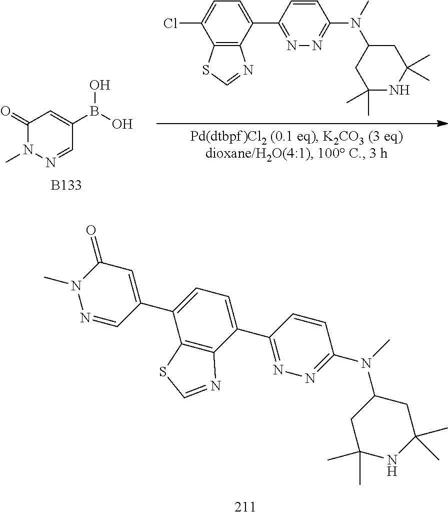

A mixture of 6-(7-chloro-1,3-benzothiazol-4-yl)-N-Methyl-N-(2,2,6,6-tetramethylpiperidin-4-yl) pyridazin-3-amine (100 mg, 0.240 mmol, 1.00 equiv), 1-methyl-6-oxopyridazin-4-ylboronic acid (74.01 mg, 0.480 mmol, 2 equiv), Pd(dtpdf)Cl2 (19.58 mg, 0.024 mmol, 0.1 equiv), and K₂CO₃ (99.67 mg, 0.720 mmol, 3 equiv) in dioxane (0.8 mL,) and water (0.2 mL) was stirred at 90° C. for 3 h under nitrogen atmosphere. The resulting mixture was concentrated under vacuum to give a residue. The residue was purified by silica gel column chromatography with CH₂Cl₂/MeOH (10:1), followed by prep-HPLC (Condition 7, Gradient 1) to afford 2-methyl-5-[6-(piperidin-4-yl) pyrido[3,2-d] pyrimidin-2-yl] indazol-4-ol (0.8 mg, 1.6%) as a solid. LCMS (ES, m/z): 490 [M+H]⁺. ¹H NMR (400 MHz, DMSO-d6) δ 9.54 (s, 1H), 8.52 (d, J=9.7 Hz, 1H), 8.34 (d, J=1.5 Hz, 2H), 7.93 (d, 1H), 7.28 (s, 1H), 7.20 (d, J=9.8 Hz, 1H), 5.16 (s, 1H), 3.76 (s, 3H), 2.98 (s, 3H), 1.55 (d, J=11.1 Hz, 2H), 1.46 (t, J=12.1 Hz, 2H), 1.28 (s, 6H), 1.11 (s, 6H).

### Example 40: Synthesis of Compound 212

### Synthesis of Intermediate B134

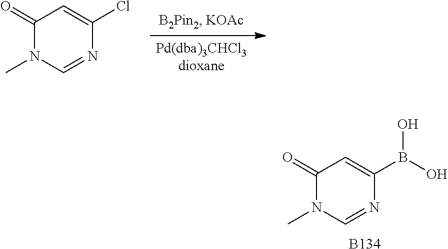

To a solution of 6-chloro-3-methylpyrimidin-4-one (100 mg, 0.692 mmol, 1.00 equiv), B₂pin₂ (351.33 mg, 1.384 mmol, 2 equiv), KOAc (135.78 mg, 1.384 mmol, 2.00 equiv), and X-Phos (32.98 mg, 0.069 mmol, 0.10 equiv) in dioxane (1.00 mL, 11.81 mmol, 17.06 equiv) was added Pd₂(dba)₃CHCl₃ (35.80 mg, 0.035 mmol, 0.05 equiv) under nitrogen atmosphere. The reaction mixture was stirred at 90° C. for 5 h. LCMS (ES, m/z): 155 [M+H]⁺.

### Synthesis of Compound 212

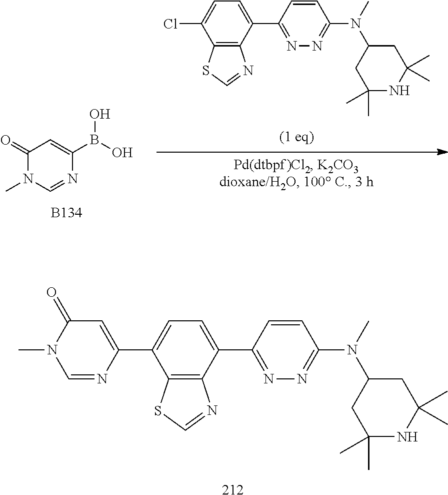

A mixture of 6-(7-chloro-1,3-benzothiazol-4-yl)-N-methyl-N-(2,2,6,6-tetramethylpiperidin-4-yl)pyridazin-3-amine (100 mg, 0.240 mmol, 1.00 equiv), (1-methyl-6-oxo-1,6-dihydropyridazin-4-yl)boronic acid (74.01 mg, 0.48 mmol, 2 equiv), Pd(dtpdf)Cl₂ (19.58 mg, 0.02 mmol, 0.1 equiv) and K₂CO₃ (99.67 mg, 0.72 mmol, 3 equiv) in dioxane (0.8 mL) and H₂O (0.2 mL, 11.10 mmol, 46.18 equiv) was stirred at 90° C. for 3 h under nitrogen atmosphere. The resulting mixture was concentrated under vacuum to give a residue. The residue was purified by silica gel column chromatography with CH₂Cl₂/MeOH (10:1), followed by prep-HPLC (Condition 7, Gradient 1) to afford 2-methyl-5-[6-(piperidin-4-yl) pyrido[3,2-d] pyrimidin-2-yl] indazol-4-ol (0.8 mg, 1.6%) as a solid. LCMS (ES, m/z): 463 [M+H]⁺. ¹H NMR (400 MHz, DMSO-d6) δ 9.54 (s, 1H), 8.78 (s, 1H), 8.52 (d, J=9.7 Hz, 1H), 8.34 (d, J=1.5 Hz, 2H), 7.28 (s, 1H), 7.20 (d, J=9.8 Hz, 1H), 5.16 (s, 1H), 3.51 (s, 3H), 2.98 (s, 3H), 1.55 (d, J=11.1 Hz, 2H), 1.46 (t, J=12.1 Hz, 2H), 1.28 (s, 6H), 1.11 (s, 6H).

### Example 41: Synthesis of Compound 213

### Synthesis of Intermediate B135

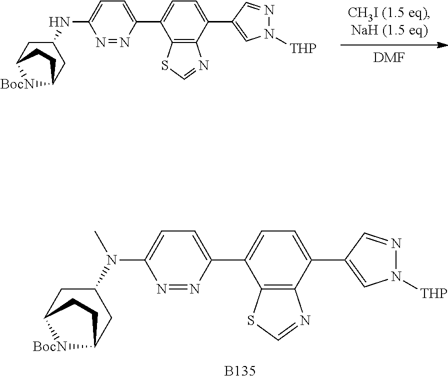

To a solution of tert-butyl (exo)-3-[(6-{4-[1-(oxan-2-yl)pyrazol-4-yl]-1,3-benzothiazol-7-yl}pyridazin-3-yl)amino]-8-azabicyclo[3.2.1]octane-8-carboxylate (150 mg, 0.255 mmol, 1.00 equiv) in DMF was added sodium hydride (60% in oil, 5 mg) at 0° C. The reaction mixture was stirred for 15 min. CH₃I (43.47 mg, 0.306 mmol, 1.2 equiv) was added and the reaction mixture was allowed to warm to room temperature and stirred for an additional 1 h. The reaction mixture was quenched with water and extracted with DCM (3×25 mL). The combined organic layers were concentrated under reduced pressure to afford tert-butyl (exo)-3-[methyl(6-{4-[1-(oxan-2-yl)pyrazol-4-yl]-1,3-benzothiazol-7-yl}pyridazin-3-yl)amino]-8-azabicyclo[3.2.1]octane-8-carboxylate (100 mg, 65.11%) as a solid. LCMS (ES, m/z): 602 [M+H]⁺.

### Synthesis of Compound 213

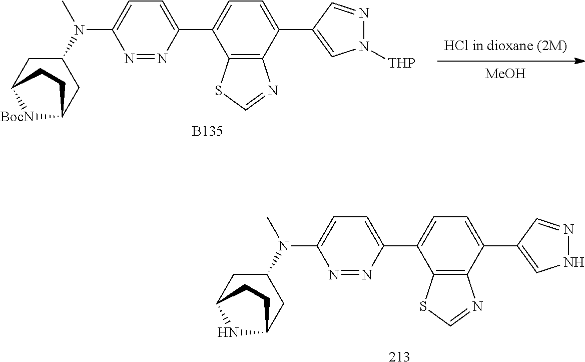

A mixture of tert-butyl (exo)-3-[methyl(6-{4-[1-(oxan-2-yl)pyrazol-4-yl]-1,3-benzothiazol-7-yl}pyridazin-3-yl)amino]-8-azabicyclo[3.2.1]octane-8-carboxylate (120 mg, 0.199 mmol, 1.00 equiv), HCl in dioxane (2 M), and methanol (2 mL, 49.398 mmol, 247.72 equiv) was stirred for 1 h at room temperature. The reaction mixture was concentrated under reduced pressure to give a residue. The residue was purified by reverse flash chromatography (Column, silica gel; Mobile Phase, acetonitrile in water; Gradient: 10% to 50% in 10 min; detector, UV 254 nm) to afford (exo)-N-methyl-N-{6-[4-(1H-pyrazol-4-yl)-1,3-benzothiazol-7-yl]pyridazin-3-yl}-8-azabicyclo[3.2.1]octan-3-amine (10.3 mg, 12.01%) as a solid. LCMS (ES, m/z): 418 [M+H]⁺. ¹H NMR (400 MHz, DMSO-d₆) δ 13.09 (s, 1H), 9.51 (s, 1H), 8.54 (s, 2H), 8.24 (d, J=9.9 Hz, 1H), 8.10 (d, J=8.1 Hz, 1H), 7.98 (d, J=7.9 Hz, 1H), 7.26 (d, J=9.8 Hz, 1H), 5.00 (s, 1H), 3.51 (d, J=3.9 Hz, 2H), 2.98 (s, 3H), 1.87-1.77 (m, 2H), 1.77 (s, 4H), 1.61-1.52 (m, 2H).

### Example 42: Synthesis of Compound 214

### Synthesis of Intermediate B136

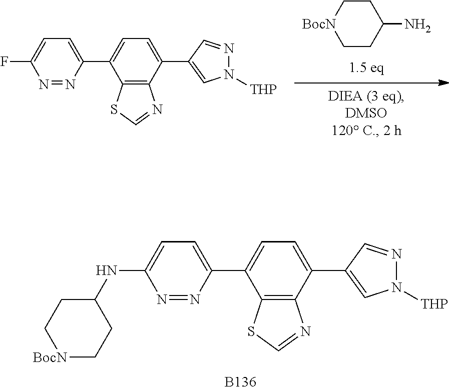

A mixture of 7-(6-chloropyridazin-3-yl)-4-[1-(oxan-2-yl)pyrazol-4-yl]-1,3-benzothiazole (400 mg, 1.005 mmol, 1.00 equiv), tert-butyl 4-aminopiperidine-1-carboxylate (302.02 mg, 1.507 mmol, 1.5 equiv), DIEA (389.80 mg, 3.015 mmol, 3 equiv), and DMSO (20 mL, 281.571 mmol, 280.08 equiv) was stirred for 2 h at 120° C. The reaction mixture was cooled to room temperature, then quenched with water and extracted with ethyl acetate (3×20 mL). The combined organic layers were washed with brine (2×10 mL), dried over anhydrous Na₂SO₄, and filtered. After filtration, the filtrate was concentrated under reduced pressure to afford tert-butyl 4-[(6-{4-[1-(oxan-2-yl)pyrazol-4-yl]-1,3-benzothiazol-7-yl}pyridazin-3-yl)amino]piperidine-1-carboxylate (500 mg, 88.54%) as a solid. LCMS (ES, m/z): 562 [M+H]⁺.

### Synthesis of Compound 214

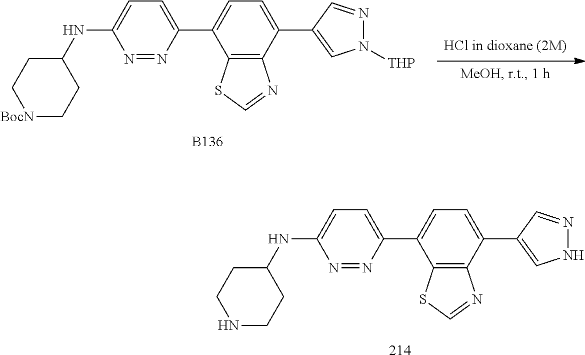

A mixture of tert-butyl 4-[(6-{4-[1-(oxan-2-yl)pyrazol-4-yl]-1,3-benzothiazol-7-yl} pyridazin-3-yl)amino]piperidine-1-carboxylate (120 mg), HCl(gas) in 1,4-dioxane (0.5 mL), and methanol (0.5 mL) was stirred for 1 h at room temperature. The resulting mixture was concentrated under reduced pressure to give a residue. The residue was purified by reverse flash chromatography (Column, silica gel; mobile phase, acetonitrile in water; gradient, 10% to 50% in 10 min; detector, UV 254 nm) to afford N-(piperidin-4-yl)-6-[4-(1H-pyrazol-4-yl)-1,3-benzothiazol-7-yl]pyridazin-3-amine (22 mg, 27.28%) as a solid. LCMS (ES, m/z): 378 [M+H]⁺. ¹H NMR (400 MHz, DMSO-d₆) δ 13.01 (s, 1H), 9.51 (s, 1H), 8.54 (s, 2H), 8.16 (d, J=9.5 Hz, 1H), 8.05 (d, J=8.1 Hz, 1H), 7.98 (d, J=7.9 Hz, 1H), 7.06 (d, J=7.6 Hz, 1H), 6.98 (d, J=9.5 Hz, 1H), 4.07 (s, 2H), 3.03 (d, J=12.4 Hz, 2H), 2.66 (t, J=11.4 Hz, 2H), 1.99 (d, J=12.2 Hz, 2H), 1.46-1.37 (m, 1H), 1.36 (dd, J=11.5, 3.7 Hz, 1H).

### Example 43: Synthesis of Compound 222

### Synthesis of Intermediate B137

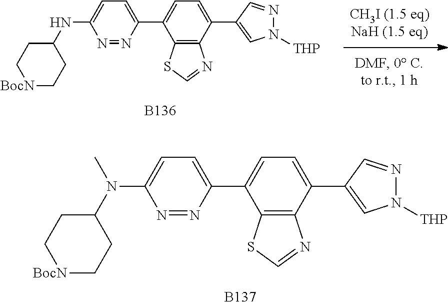

To a solution of tert-butyl 4-[(6-{4-[1-(oxan-2-yl)pyrazol-4-yl]-1,3-benzothiazol-7-yl}pyridazin-3-yl)amino]piperidine-1-carboxylate (120 mg, 0.214 mmol, 1.00 equiv) in DMF was added sodium hydride (60% in oil, 1 mg) at 0° C. The reaction mixture was stirred for 15 min. CH₃I (45.48 mg, 0.321 mmol, 1.5 equiv) was added and the mixture was allowed to warm to room temperature and stirred for an additional 1 h. The reaction mixture was quenched with water at room temperature and extracted with ethyl acetate (3×10 mL). The combined organic layers were washed with brine (2×5 mL), dried over anhydrous Na₂SO₄, and filtered. After filtration, the filtrate was concentrated under reduced pressure to afford tert-butyl 4-[methyl(6-{4-[1-(oxan-2-yl)pyrazol-4-yl]-1,3-benzothiazol-7-yl}pyridazin-3-yl)amino]piperidine-1-carboxylate (100 mg, 81.30%) as a solid. LCMS (ES, m/z): 576 [M+H]⁺.

### Synthesis of Compound 222

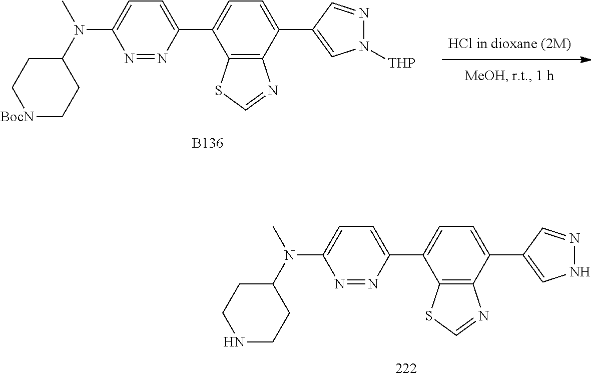

Tert-butyl 4-[methyl(6-{4-[1-(oxan-2-yl)pyrazol-4-yl]-1,3-benzothiazol-7-yl}pyridazin-3-yl)amino]piperidine-1-carboxylate (100 mg, 0.174 mmol, 1.00 equiv), MeOH (2 mL, 49.398 mmol, 284.40 equiv), and HCl(gas) in 1,4-dioxane (2 mL) were combined at room temperature. The resulting mixture was stirred for 1 h at room temperature, then concentrated under reduced pressure to give a residue. The residue was purified by reverse flash chromatography (column, silica gel; mobile phase, acetonitrile in water; gradient, 10% to 50% in 10 min; detector, UV 254 nm), followed by prep-HPLC (Condition 7, Gradient 1) to afford N-methyl-N-(piperidin-4-yl)-6-[4-(1H-pyrazol-4-yl)-1,3-benzothiazol-7-yl]pyridazin-3-amine (10 mg, 14.71%) as a solid. LCMS (ES, m/z): 392[M+H]^{+ 1}H NMR (400 MHz, DMSO-d₆) δ 13.09 (s, 1H), 9.52 (s, 1H), 8.67-8.42 (s, 2H), 8.27 (d, J=9.8 Hz, 1H), 8.12 (d, J=8.0 Hz, 1H), 7.99 (d, J=8.0 Hz, 1H), 7.30 (d, J=9.8 Hz, 1H), 4.69 (s, 1H), 3.09 (d, J=12.3 Hz, 2H), 3.02 (s, 3H), 2.72 (d, J=11.8 Hz, 2H), 1.72 (t, J=10.9 Hz, 2H), 1.64 (d, J=11.6 Hz, 2H).

### Example 44: Synthesis of Compound 223

### Synthesis of Intermediate B137

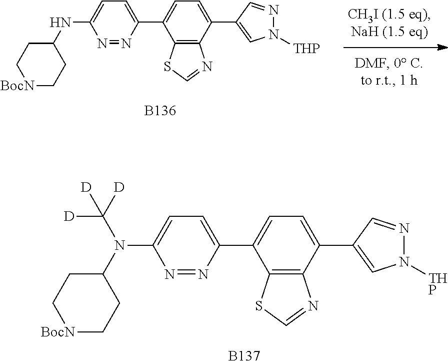

To a solution of tert-butyl 4-[(6-{4-[1-(oxan-2-yl)pyrazol-4-yl]-1,3-benzothiazol-7-yl}pyridazin-3-yl) amino]piperidine-1-carboxylate (120 mg, 0.214 mmol, 1.00 equiv) in DMF was added sodium hydride (60% in oil, 10 mg) at 0° C. The reaction mixture was stirred for 15 min. CD₃I (46.45 mg, 0.321 mmol, 1.5 equiv) was added and the reaction mixture was allowed to warm to room temperature and stirred for 1 h. The reaction mixture was quenched with water at room temperature and extracted with ethyl acetate (3×20 mL). The combined organic layers were washed with brine (2×10 mL), dried over anhydrous Na₂SO₄, and filtered. After filtration, the filtrate was concentrated under reduced pressure to afford tert-butyl 4-[(D3)methyl(6-{4-[1-(oxan-2-yl)pyrazol-4-yl]-1,3-benzothiazol-7-yl} pyridazin-3-yl)amino]piperidine-1-carboxylate (100 mg, 80.88%) as a solid. LCMS (ES, m/z): 579 [M+H]⁺.

### Synthesis of Compound 223

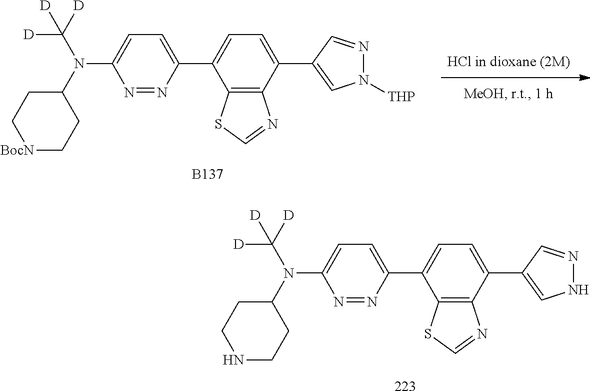

A mixture of tert-butyl 4-[(2H3)methyl(6-{4-[1-(oxan-2-yl)pyrazol-4-yl]-1,3-benzothiazol-7-yl}pyridazin-3-yl)amino]piperidine-1-carboxylate (100 mg), MeOH (2 mL), and HCl (gas) in 1,4-dioxane (2 mL) was stirred for 1 h at room temperature. The resulting mixture was concentrated under reduced pressure to give a residue. The residue was purified by reverse flash chromatography (column, silica gel; mobile phase, acetonitrile in water; gradient, 10% to 50% in 10 min; detector, UV 254 nm) to afford N-(D3)methyl-N-(piperidin-4-yl)-6-[4-(1H-pyrazol-4-yl)-1,3-benzothiazol-7-yl] pyridazin-3-amine (10.5 mg) as a solid. LCMS (ES, m/z): 395[M+H]⁺. 1H NMR (400 MHz, DMSO-d₆) δ 13.08 (s, 1H), 9.51 (s, 1H), 8.7-8.4 (s, 2H), 8.26 (d, J=9.8 Hz, 1H), 8.11 (d, J=8.1 Hz, 1H), 7.98 (d, J=8.0 Hz, 1H), 7.29 (d, J=9.8 Hz, 1H), 4.67 (s, 1H), 3.08 (d, J=12.1 Hz, 2H), 2.75-2.64 (m, 2H), 1.78-1.60 (m, 4H).

### Example 45: Synthesis of Compound 224

### Synthesis of Intermediate B138

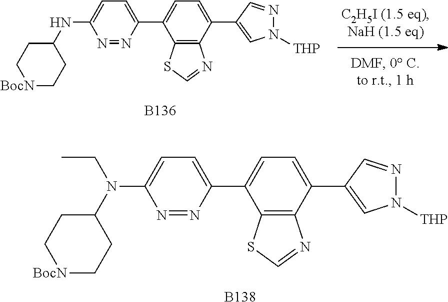

To a solution of tert-butyl 4-[(6-{4-[1-(oxan-2-yl)pyrazol-4-yl]-1,3-benzothiazol-7-yl}pyridazin-3-yl) amino]piperidine-1-carboxylate (120 mg, 0.214 mmol, 1.00 equiv) in DMF was added sodium hydride (60% in oil, 10 mg) at 0° C. The mixture was stirred for 15 min. C₂H5I (46.45 mg, 0.321 mmol, 1.5 equiv) was added. The reaction mixture was allowed to warm to room temperature and stirred for an additional 1 h, then quenched with water at room temperature and extracted with ethyl acetate (3×20 mL). The combined organic layers were washed with brine (2×10 mL), dried over anhydrous Na₂SO₄, and filtered. After filtration, the filtrate was concentrated under reduced pressure to afford tert-butyl 4-[ethyl(6-{4-(1-(tetrahydro-2H-pyran-2-yl)benzo[d]thiazol-7-yl}pyridazin-3-yl) amino]piperidine-1-carboxylate (100 mg, 80.88%) as a solid. LCMS (ES, m/z): 590 [M+H]⁺.

### Synthesis of Compound 224

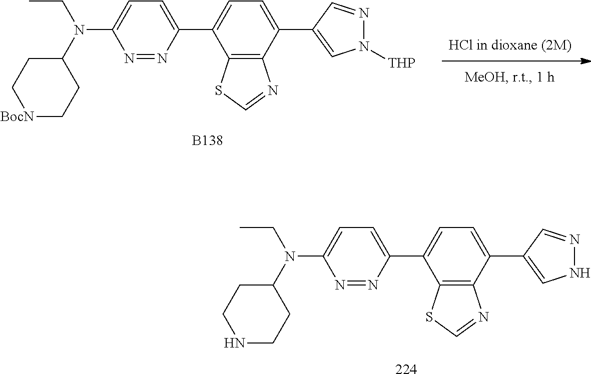

A mixture of tert-butyl 4-[ethyl(6-{4-[1-(oxan-2-yl)pyrazol-4-yl]-1,3-benzothiazol-7-yl}pyridazin-3-yl)amino]piperidine-1-carboxylate (100 mg, 0.170 mmol, 1.00 equiv), methanol (2 mL, 49.398 mmol, 291.33 equiv), and HCl (gas) in 1,4-dioxane (2 mL, 35.035 mmol, 206.62 equiv) was stirred for 1 h at room temperature. The resulting mixture was concentrated under reduced pressure to give a residue. The residue was purified by reverse flash chromatography (column, silica gel; mobile phase, acetonitrile in water; gradient, 10% to 50% in 10 min; detector, UV 254 nm) to afford N-ethyl-N-(piperidin-4-yl)-6-[4-(1H-pyrazol-4-yl)-1,3-benzothiazol-7-yl]pyridazin-3-amine (12 mg, 17.45%) as a solid. LCMS (ES, m/z): 406[M+H]⁺. ¹H NMR (400 MHz, DMSO-d₆) δ 13.08 (s, 1H), 9.51 (s, 1H), 8.54 (s, 2H), 8.23 (d, J=10.0 Hz, 1H), 8.10 (d, J=7.9 Hz, 1H), 7.98 (d, J=8.0 Hz, 1H), 7.23 (d, J=10.0 Hz, 1H), 4.69-4.62 (m, 1H), 3.56 (q, J=7.5, 6.9 Hz, 2H), 3.05 (d, J=12.0 Hz, 2H), 2.64 (t, J=6.6 Hz, 2H), 1.68 (s, 4H), 1.19 (t, J=6.9 Hz, 3H).

### Example 46: Synthesis of Compound 186

### Synthesis of Intermediate B139

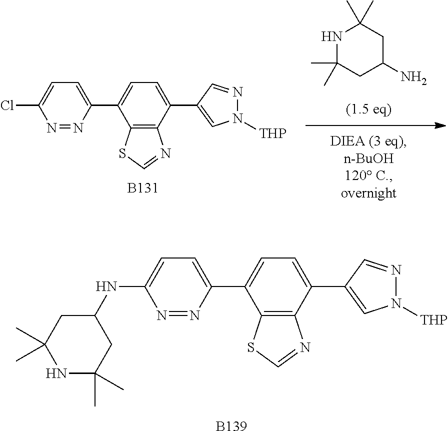

A mixture of 7-(6-chloropyridazin-3-yl)-4-[1-(oxan-2-yl)pyrazol-4-yl]-1,3-benzothiazole (800 mg, 2.011 mmol, 1.00 equiv), 2,2,6,6-tetramethylpiperidin-4-amine (471.32 mg, 3.017 mmol, 1.5 equiv), DIEA (779.59 mg, 6.033 mmol, 3 equiv), and n-BuOH (40.00 mL, 437.674 mmol, 217.64 equiv) was stirred overnight at 120° C. The reaction mixture was quenched with water at room temperature and extracted with ethyl acetate (3×10 mL). The combined organic layers were washed with brine (2×10 mL), dried over anhydrous Na₂SO₄, and filtered. After filtration, the filtrate was concentrated under reduced pressure to afford 6-{4-[1-(oxan-2-yl)pyrazol-4-yl]-1,3-benzothiazol-7-yl}-N-(2,2,6,6-tetramethylpiperidin-4-yl)pyridazin-3-amine (80 mg, 7.69%) as a solid. LCMS (ES, m/z): 518 [M+H]⁺.

### Synthesis of Compound 186

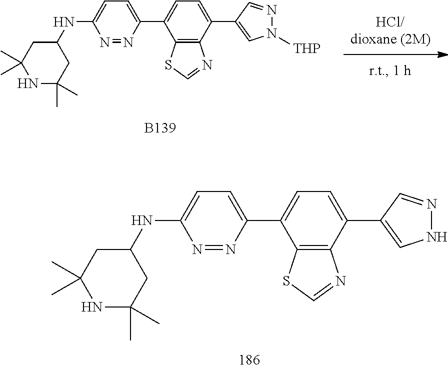

A mixture of 6-[4-[1-(oxan-2-yl)pyrazol-4-yl]-1,3-benzothiazol-7-yl]-N-(2,2,6,6-tetramethylpiperidin-4-yl)pyridazin-3-amine (70.00 mg), HCl (gas) in 1,4-dioxane (2.00 mL), and methanol (2.00 mL) was stirred for 1 h at room temperature. The resulting mixture was concentrated under reduced pressure to give a residue. The residue was purified by reverse flash chromatography (column, silica gel; mobile phase, acetonitrile in water; gradient, 10% to 50% in 10 min; detector, UV 254 nm), followed by prep-HPLC (Condition 7, Gradient 1) to afford 6-[4-(1H-pyrazol-4-yl)-1,3-benzothiazol-7-yl]-N-(2,2,6,6-tetramethylpiperidin-4-yl)pyridazin-3-amine (10 mg) as a solid. LCMS (ES, m/z): 433 [M+H]⁺. ¹H NMR (400 MHz, DMSO-d₆) δ 13.08 (s, 1H), 9.50 (s, 1H), 8.53 (s, 2H), 8.15 (d, J=9.6 Hz, 1H), 8.05 (d, J=8.1 Hz, 1H), 7.97 (d, J=7.9 Hz, 1H), 6.96 (d, J=9.5 Hz, 1H), 6.91 (d, J=7.8 Hz, 1H), 4.46 (dt, J=7.9, 6.0 Hz, 1H), 1.93 (dd, J=12.3, 3.7 Hz, 2H), 1.65 (s, 1H), 1.26 (s, 6H), 1.08 (s, 6H), 1.07 (t, J=12.1 Hz, 2H).

### Example 47: Synthesis of Compound 231

### Synthesis of Intermediate B140

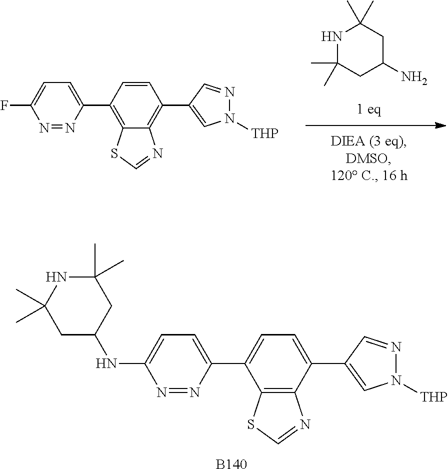

A mixture of 7-(6-fluoropyridazin-3-yl)-4-[1-(oxan-2-yl)pyrazol-4-yl]-1,3-benzothiazole (600 mg, 1.542 mmol, 1.00 equiv), 2,2,6,6-tetramethylpiperidin-4-amine (240.91 mg, 1.542 mmol, 1 equiv), and DIEA (597.71 mg, 4.626 mmol, 3 equiv) in DMSO (5.88 mL) was stirred for 16 h at 120° C. The reaction mixture was cooled to room temperature, then partitioned between ethyl acetate and water, and extracted with ethyl acetate (1×30 mL). The combined organic layers were washed with DMSO (3×30 mL), dried over anhydrous Na₂SO₄, and filtered. After filtration, the filtrate was concentrated under reduced pressure to afford 6-{4-[1-(oxan-2-yl)pyrazol-4-yl]-1,3-benzothiazol-7-yl}-N-(2,2,6,6-tetramethylpiperidin-4-yl)pyridazin-3-amine (416 mg, 52.13%) as a solid. LCMS (ES, m/z): 518 [M+H]⁺.

### Synthesis of Intermediate B141

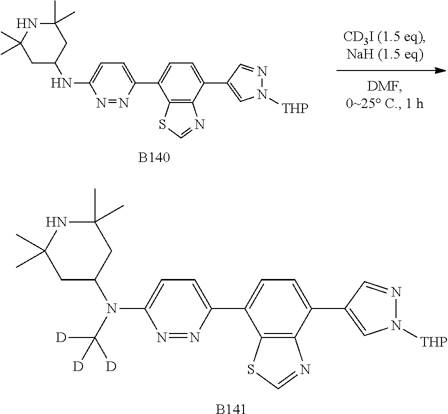

A solution of 6-{4-[1-(oxan-2-yl)pyrazol-4-yl]-1,3-benzothiazol-7-yl}-N-(2,2,6,6-tetramethylpiperidin-4-yl)pyridazin-3-amine (60 mg, 0.114 mmol, 1.00 equiv) in DMF (1 mL, 12.922 mmol, 113.77 equiv) was treated with NaH (4.09 mg, 0.171 mmol, 1.5 equiv). The reaction mixture was stirred for 30 min at 0° C. CD₃I (24.70 mg, 0.171 mmol, 1.5 equiv) was added dropwise at 0-room temperature. The resulting mixture was poured into water (30 mL), then extracted with ethyl acetate (1×30 mL). The combined organic layers were washed with DMF (3×30 mL), dried over anhydrous Na₂SO₄, and filtered. After filtration, the filtrate was concentrated under reduced pressure to afford N-(2H3)methyl-6-{4-[1-(oxan-2-yl)pyrazol-4-yl]-1,3-benzothiazol-7-yl}-N-(2,2,6,6-tetramethylpiperidin-4-yl)pyridazin-3-amine (22 mg, 36.22%) as a solid. LCMS (ES, m/z): 535 [M+H]⁺.

### Synthesis of Compound 231

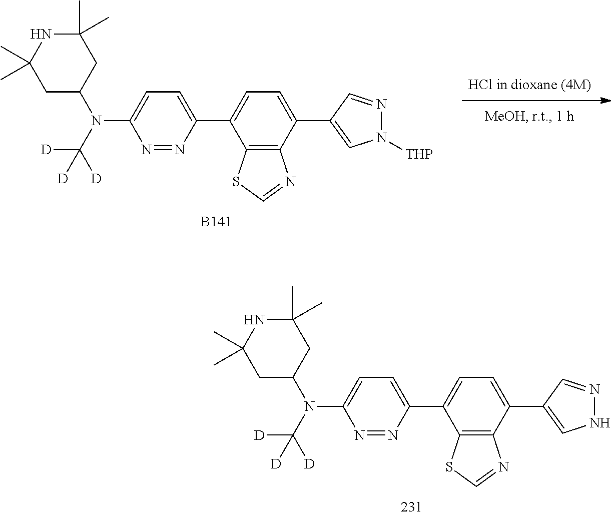

A mixture of N-(2H3)methyl-6-{4-[1-(oxan-2-yl)pyrazol-4-yl]-1,3-benzothiazol-7-yl}-N-(2,2,6,6-tetramethylpiperidin-4-yl)pyridazin-3-amine (80 mg, 0.150 mmol, 1.00 equiv), methanol (1 mL), and HCl (gas) in 1,4-dioxane (4M, 1 mL) was stirred for 1 h at room temperature. The resulting mixture was concentrated under reduced pressure to give a residue. The residue was purified by prep-HPLC (Condition 7, Gradient 2) to afford N-(2H3)methyl-6-[4-(1H-pyrazol-4-yl)-1,3-benzothiazol-7-yl]-N-(2,2,6,6-tetramethylpiperidin-4-yl)pyridazin-3-amine (8.2 mg, 11.91%) as a solid. LCMS (ES, m/z): 451 [M+H]⁺. ¹H NMR (400 MHz, DMSO-d₆) δ 13.09 (s, 1H), 9.51 (s, 1H), 8.68 (s, 1H), 8.40 (s, 1H), 8.27 (d, J=9.8 Hz, 1H), 8.10 (d, J=8.0 Hz, 1H), 7.99 (d, J=8.0 Hz, 1H), 7.26 (d, J=9.7 Hz, 1H), 5.00 (s, 1H), 1.56 (dd, J=12.4, 3.6 Hz, 1H), 1.46 (t, J=12.1 Hz, 2H), 1.27 (d, J=11.1 Hz, 7H), 1.11 (s, 6H), 0.07 (s, 1H).

### Example 48: Synthesis of Compound 225

### Synthesis of Intermediate B142

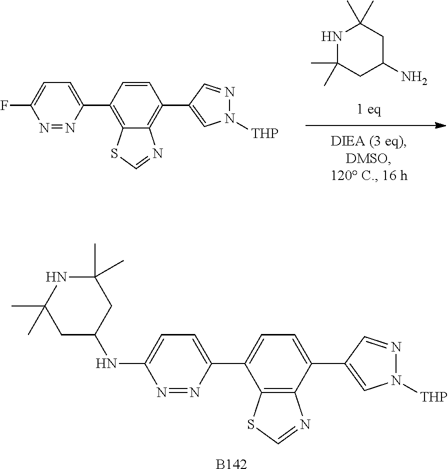

A solution of 7-(6-fluoropyridazin-3-yl)-4-[1-(oxan-2-yl)pyrazol-4-yl]-1,3-benzothiazole (600 mg, 1.542 mmol, 1.00 equiv), 2,2,6,6-tetramethylpiperidin-4-amine (240.91 mg, 1.542 mmol, 1 equiv), and DIEA (597.71 mg, 4.626 mmol, 3 equiv) in DMSO (6.00 mL) was stirred for 16 h at 120° C. The reaction mixture was cooled to room temperature. The resulting mixture was extracted with ethyl acetate (1×30 mL). The combined organic layers were washed with brine (3×30 mL), dried over anhydrous Na₂SO₄, and filtered. After filtration, the filtrate was concentrated under reduced pressure to afford 6-{4-[1-(oxan-2-yl)pyrazol-4-yl]-1,3-benzothiazol-7-yl}-N-(2,2,6,6-tetramethylpiperidin-4-yl)pyridazin-3-amine (416 mg, 52.13%) as a solid. LCMS (ES, m/z): 518 [M+H]⁺.

### Synthesis of Intermediate B143

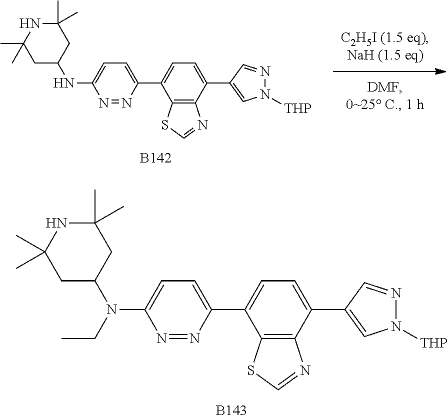

A solution of 6-{4-[1-(oxan-2-yl)pyrazol-4-yl]-1,3-benzothiazol-7-yl}-N-(2,2,6,6-tetramethylpiperidin-4-yl)pyridazin-3-amine (60 mg, 0.116 mmol, 1.00 equiv) in DMF (0.60 mL,) was treated with NaH (4.17 mg, 0.174 mmol, 1.5 equiv). The reaction mixture was stirred for 30 min at 0° C. under nitrogen atmosphere. Iodoethane (27.11 mg, 0.174 mmol, 1.50 equiv) was added dropwise at 0° C., and the resulting mixture was stirred for 1 h at room temperature. The resulting mixture was poured into water (5 mL) and extracted with ethyl acetate (2×5 mL). The combined organic layers were washed with brine (3×5 mL), dried over anhydrous Na₂SO₄, and filtered. After filtration, the filtrate was concentrated under reduced pressure to afford N-ethyl-6-{4-[1-(oxan-2-yl)pyrazol-4-yl]-1,3-benzothiazol-7-yl}-N-(2,2,6,6-tetramethylpiperidin-4-yl)pyridazin-3-amine (27 mg, 42.69%) as a solid. LCMS (ES, m/z): 546 [M+H]⁺.

### Synthesis of Compound 225

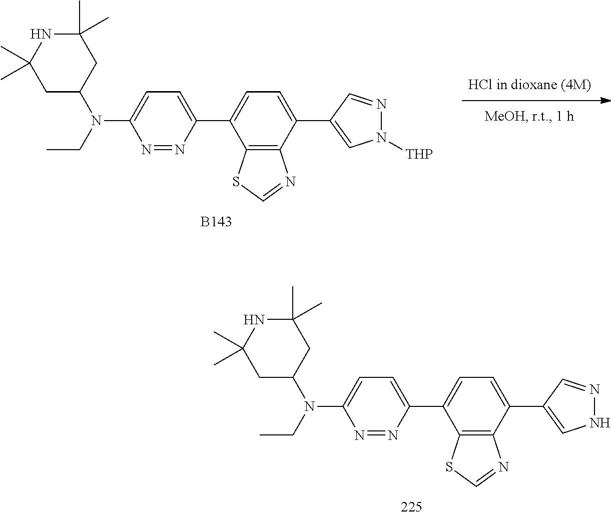

A mixture of N-(2,2-dimethylpiperidin-4-yl)-N-ethyl-6-{4-[1-(oxan-2-yl)pyrazol-4-yl]-1,3-benzothiazol-7-yl}pyridazin-3-amine (27 mg, 0.052 mmol, 1.00 equiv), methanol (1 mL), and HCl (gas) in 1,4-dioxane (1 mL) was stirred for 1 h at room temperature. The resulting mixture was concentrated under reduced pressure to give a residue. The residue was purified by prep-HPLC (Condition 8, Gradient 1, Gradient 2) to afford N-ethyl-6-[4-(1H-pyrazol-4-yl)-1,3-benzothiazol-7-yl]-N-(2,2,6,6-tetramethylpiperidin-4-yl)pyridazin-3-amine (3.1 mg, 12.88%) as a solid. LCMS (ES, m/z): 462 [M+H]⁺. ¹H NMR (400 MHz, DMSO-d₆) δ 13.09 (s, 1H), 9.50 (s, 1H), 8.68 (s, 1H), 8.40 (s, 1H), 8.25 (d, J=9.9 Hz, 1H), 8.09 (d, J=8.1 Hz, 1H), 7.98 (d, J=8.0 Hz, 1H), 7.20 (d, J=9.9 Hz, 1H), 5.02 (s, 1H), 3.56 (d, J=7.2 Hz, 2H), 1.61 (d, J=11.0 Hz, 2H), 1.45 (t, J=12.2 Hz, 2H), 1.29 (s, 6H), 1.19 (t, J=6.9 Hz, 3H), 1.11 (s, 6H).

### Example 49: Synthesis of Compound 202

### Synthesis of Intermediate B144

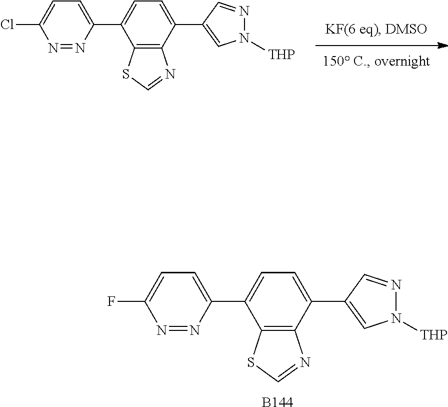

A mixture of 7-(6-chloropyridazin-3-yl)-4-[1-(oxan-2-yl)pyrazol-4-yl]-1,3-benzothiazole (1.80 g, 4.524 mmol, 1.00 equiv) and KF (1.58 g, 0.027 mmol, 6 equiv) in DMSO (6.00 mL, 84.471 mmol, 18.67 equiv) was stirred for 8 h at 150° C. The reaction mixture was quenched with water at room temperature and extracted with ethyl acetate (3×10 mL). The combined organic layers were washed with brine (2×10 mL), dried over anhydrous Na₂SO₄, and filtered. After filtration, the filtrate was concentrated under reduced pressure to afford 7-(6-fluoropyridazin-3-yl)-4-[1-(oxan-2-yl)pyrazol-4-yl]-1,3-benzothiazole (1.3 g, 75.34%) as a solid. LCMS (ES, m/z): 382 [M+H]⁺.

### Synthesis of Intermediate B145

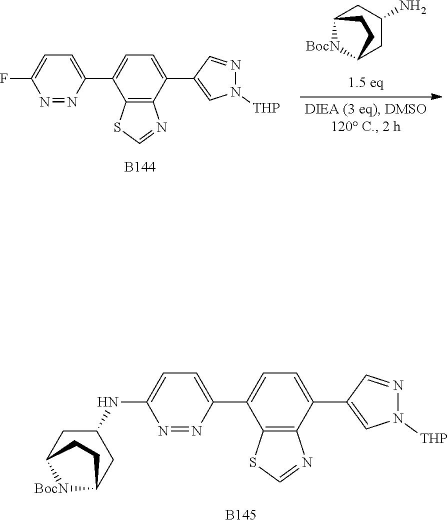

The mixture of 7-(6-fluoropyridazin-3-yl)-4-[1-(oxan-2-yl)pyrazol-4-yl]-1,3-benzothiazole (800.00 mg, 2.097 mmol, 1.00 equiv), tert-butyl (exo-)-3-amino-8-azabicyclo[3.2.1]octane-8-carboxylate (712.02 mg, 3.146 mmol, 1.50 equiv), and DIEA (813.21 mg, 6.292 mmol, 3 equiv), in DMSO (5.00 mL, 63.989 mmol, 33.56 equiv) was stirred overnight at 120° C. The reaction mixture was quenched with water at room temperature and extracted with ethyl acetate (3×10 mL). The combined organic layers were washed with brine (2×10 mL), dried over anhydrous Na₂SO₄, and filtered. After filtration, the filtrate was concentrated under reduced pressure to afford tert-butyl (exo-)-3-[(6-[4-[1-(oxan-2-yl)pyrazol-4-yl]-1,3-benzothiazol-7-yl]pyridazin-3-yl)amino]-8-azabicyclo[3.2.1]octane-8-carboxylate (1.3 g, 105.46%) as a solid. LCMS (ES, m z): 588 [M+H]⁺.

### Synthesis of Compound 202

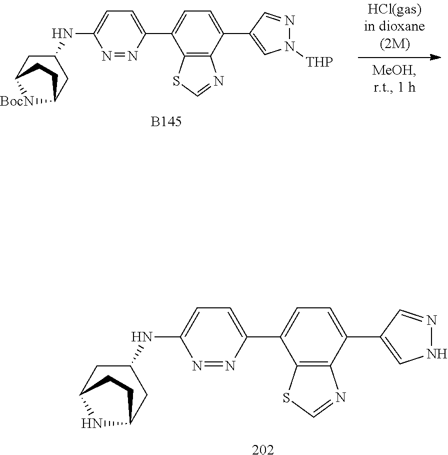

A mixture of tert-butyl(exo-)-3-[(6-[4-[1-(oxan-2-yl)pyrazol-4-yl]-1,3-benzothiazol-7-yl]pyridazin-3-yl)amino]-8-azabicyclo[3.2.1]octane-8-carboxylate (100.00 mg), HCl (gas) in 1,4-dioxane (3.00 mL), and methanol (3.00 mL) was stirred for 30 min at room temperature. The resulting mixture was concentrated under reduced pressure to give a residue. The residue was purified by reverse flash chromatography (column, silica gel; mobile phase, acetonitrile in water; gradient, 10% to 50% in 10 min; detector, UV 254 nm), followed by prep-HPLC (Condition 7, Gradient 1) to afford (exo-)-N-[6-[4-(1H-pyrazol-4-yl)-1,3-benzothiazol-7-yl]pyridazin-3-yl]-8-azabicyclo[3.2.1]octan-3-amine (17.0 mg) as a solid. LCMS (ES, m/z): 404 [M+H]⁺. ¹H NMR (400 MHz, DMSO-d₆) δ 9.49 (s, 1H), 8.52 (s, 2H), 8.12 (d, J=9.6 Hz, 1H), 8.03 (d, J=8.1 Hz, 1H), 7.97 (d, J=8.0 Hz, 1H), 6.95 (d, J=9.5 Hz, 1H), 4.35 (dt, J=11.4, 6.0 Hz, 1H), 1.96 (dt, J=12.7, 3.8 Hz, 2H), 1.76 (s, 4H), 1.51-1.40 (m, 2H).

### Example 50: Synthesis of Compound 210

### Synthesis of Intermediate B146

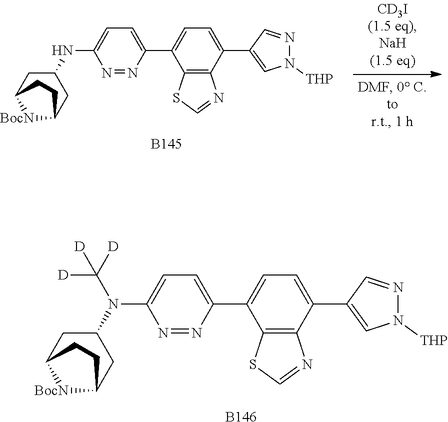

To a solution of tert-butyl (exo-)-3-[(6-[4-[1-(oxan-2-yl)pyrazol-4-yl]-1,3-benzothiazol-7-yl]pyridazin-3-yl) amino]-8-azabicyclo[3.2.1]octane-8-carboxylate (150.00 mg, 0.255 mmol, 1.00 equiv) in DMF was added sodium hydride (60% in oil, 3 mg) at 0° C. The mixture was stirred for 15 min. CD₃I (55.49 mg, 0.383 mmol, 1.5 equiv) was added and the mixture was allowed to warm to room temperature and stirred for 2 h. The reaction mixture was quenched by water and extracted with DCM (3×25 mL). The reaction mixture was quenched with water at room temperature and extracted with ethyl acetate (3×10 mL). The combined organic layers were washed with brine (2×10 mL), dried over anhydrous Na₂SO₄, and filtered. After filtration, the filtrate was concentrated under reduced pressure to afford tert-butyl (exo-)-3-[(D3)methyl (6-[4-[1-(oxan-2-yl)pyrazol-4-yl]-1,3-benzothiazol-7-yl]pyridazin-3-yl)amino]-8-azabicyclo [3.2.1]octane-8-carboxylate (100 mg, 64.79%) as a solid. LCMS (ES, m/z): 605 [M+H]⁺.

### Synthesis of Compound 210

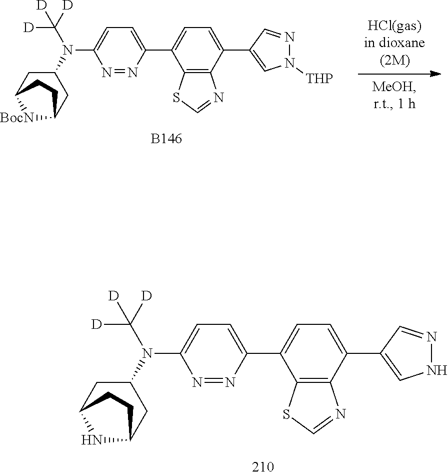

A mixture of tert-butyl (exo-)-3-[(D3)methyl (6-[4-[1-(oxan-2-yl)pyrazol-4-yl]-1,3-benzothiazol-7-yl] pyridazin-3-yl)amino]-8-azabicyclo[3.2.1]octane-8-carboxylate (10 mg), HCl (gas) in 1,4-dioxane (0.5 mL), and methanol (0.5 mL) was stirred for 1 h at room temperature. The reaction mixture was concentrated under reduced pressure to give a residue. The residue was purified by reverse flash chromatography (column, silica gel; mobile phase, acetonitrile in water; gradient, 10% to 50% in 10 min; detector, UV 254 nm), followed by prep-HPLC (Condition 7, Gradient 1) to afford (exo-)-N-(D3)methyl-N-[6-[4-(1H-pyrazol-4-yl)-1,3-benzothiazol-7-yl]pyridazin-3-yl]-8-azabicyclo [3.2.1]octan-3-amine (19.6 mg) as a solid. LCMS (ES, m/z): 420 [M+H]⁺. 1H NMR (400 MHz, DMSO-d₆) δ 13.09 (s, 1H), 9.51 (s, 1H), 8.54 (s, 2H), 8.24 (d, J=9.8 Hz, 1H), 8.10 (d, J=8.1 Hz, 1H), 7.98 (d, J=8.0 Hz, 1H), 7.25 (d, J=9.8 Hz, 1H), 5.00 (s, 1H), 3.51 (s, 2H), 1.83 (dd, J=12.2, 3.0 Hz, 2H), 1.77 (s, 4H), 1.61-1.52 (m, 2H).

### Example 51: Synthesis of Compound 203

### Synthesis of Intermediate B147

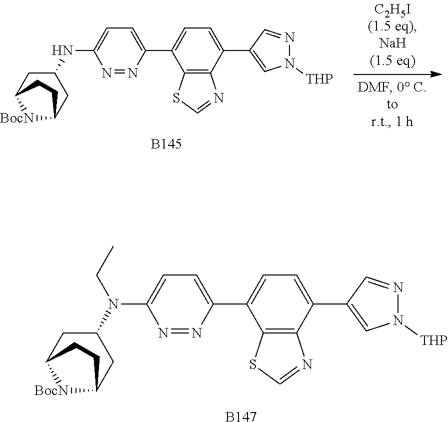

To a solution of tert-butyl (exo-)-3-[(6-[4-[1-(oxan-2-yl)pyrazol-4-yl]-1,3-benzothiazol-7-yl]pyridazin-3-yl)amino]-8-azabicyclo[3.2.1]octane-8-carboxylate (150.00 mg, 0.255 mmol, 1.00 equiv) in DMF was added sodium hydride (60% in oil, 3 mg) at 0° C. The reaction mixture was stirred for 15 min. C₂H5I (65.08 mg, 0.383 mmol, 1.5 equiv) was added and the reaction mixture was allowed to warm to room temperature, then stirred for 2 h. The reaction mixture was quenched by water and extracted with DCM (3×25 mL). The combined organic layers were washed with brine (2×10 mL), dried over anhydrous Na₂SO₄, and filtered. After filtration, the filtrate was concentrated under reduced pressure to afford tert-butyl (exo-)-3-[ethyl(6-[4-[1-(oxan-2-yl)pyrazol-4-yl]-1,3-benzothiazol-7-yl]pyridazin-3-yl)amino]-8-azabicyclo[3.2.1]octane-8-carboxylate (100 mg, 63.63%) as a solid. LCMS (ES, m/z): 616 [M+H]⁺.

### Synthesis of Compound 203

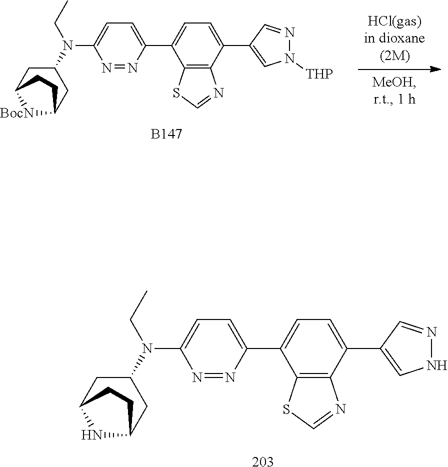

A mixture of tert-butyl (exo-)-3-[ethyl(6-[4-[1-(oxan-2-yl)pyrazol-4-yl]-1,3-benzothiazol-7-yl]pyridazin-3-yl) amino]-8-azabicyclo[3.2.1]octane-8-carboxylate (100.00 mg), HCl (gas) in 1,4-dioxane (3.00 mL), and methanol (3.00 mL) was stirred for 1 h at room temperature. The reaction mixture was concentrated under reduced pressure to give a residue. The residue was purified by reverse flash chromatography (column, silica gel; mobile phase, acetonitrile in water; gradient, 10% to 50% in 10 min; detector, UV 254 nm), followed by prep-HPLC (Condition 7, Gradient 1) to afford (exo-)-N-ethyl-N-[6-[4-(1H-pyrazol-4-yl)-1,3-benzothiazol-7-yl]pyridazin-3-yl]-8-azabicyclo[3.2.1]octan-3-amine 6 mg) as a solid. LCMS (ES, m/z): 432 [M+H]⁺. ¹H NMR (400 MHz, DMSO-d6) δ 13.09 (s, 1H), 9.51 (s, 1H), 8.54 (s, 2H), 8.21 (d, J=9.9 Hz, 1H), 8.08 (d, J=8.1 Hz, 1H), 7.98 (d, J=7.9 Hz, 1H), 7.20 (d, J=9.8 Hz, 1H), 4.96 (s, 1H), 3.53 (d, J=7.3 Hz, 1H), 3.50 (s, 3H), 1.76 (s, 6H), 1.66-1.57 (m, 2H), 1.16 (t, J=6.9 Hz, 3H).

### Example 52: Synthesis of Compound 204

### Synthesis of Intermediate B148

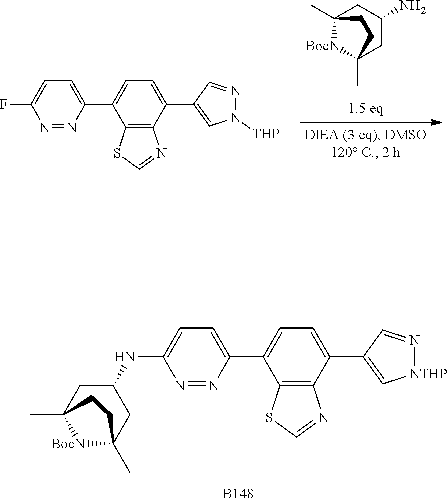

A mixture of 7-(6-fluoropyridazin-3-yl)-4-[1-(oxan-2-yl)pyrazol-4-yl]-1,3-benzothiazole (60.00 mg, 0.157 mmol, 1.00 equiv), tert-butyl (exo-)-3-amino-8-azabicyclo[3.2.1]octane-8-carboxylate (53.40 mg, 0.236 mmol, 1.5 equiv), DIEA (60.99 mg, 0.472 mmol, 3.00 equiv), and DMSO (3.00 mL, 38.396 mmol, 268.50 equiv) was stirred for 2 h at 120° C. The reaction mixture was quenched with water at room temperature, then extracted with ethyl acetate (3×10 mL). The combined organic layers were washed with brine (2×10 mL), dried over anhydrous Na₂SO₄, and filtered. After filtration, the filtrate was concentrated under reduced pressure to afford tert-butyl (exo-)-3-[(6-[4-[1-(oxan-2-yl)pyrazol-4-yl]-1,3-benzothiazol-7-yl]pyridazin-3-yl)amino]-8-azabicyclo[3.2.1]octane-8-carboxylate (60 mg, 64.90%) as a solid. LCMS (ES, m/z): 588 [M+H]⁺.

### Synthesis of Intermediate B149

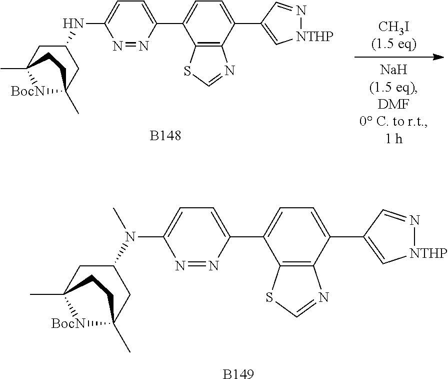

To a solution of tert-butyl (exo-)-1,5-dimethyl-3-[(6-[4-[1-(oxan-2-yl)pyrazol-4-yl]-1,3-benzothiazol-7-yl]pyridazin-3-yl)amino]-8-azabicyclo[3.2.1]octane-8-carboxylate (50.00 mg, 1.00 equiv) in DMF was added sodium hydride (60% in oil, 3 mg) at 0° C. The reaction mixture was stirred for 15 min. CH₃I (5.00 mg, 1.50 equiv) was added and the reaction mixture was allowed to warm to room temperature and stirred for an additional 1 h. The reaction mixture was quenched with water and extracted with DCM (3×25 mL) to afford tert-butyl (exo-)-1,5-dimethyl-3-[methyl(6-[4-[1-(oxan-2-yl)pyrazol-4-yl]-1,3-benzothiazol-7-yl]pyridazin-3-yl)amino]-8-azabicyclo[3.2.1]octane-8-carboxylate (41 mg) as a solid. LCMS (ES, m/z): 630 [M+H]⁺.

### Synthesis of Compound 204

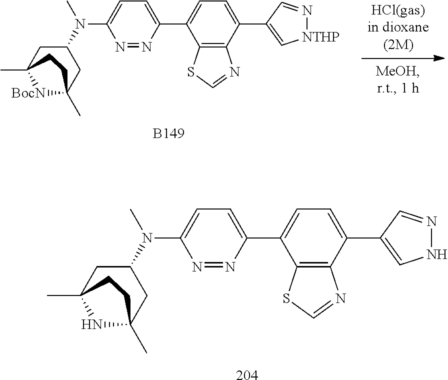

A mixture of tert-butyl (exo-)-1,5-dimethyl-3-[methyl(6-[4-[1-(oxan-2-yl)pyrazol-4-yl]-1,3-benzothiazol-7-yl]pyridazin-3-yl)amino]-8-azabicyclo[3.2.1]octane-8-carboxylate (36.00 mg), HCl (gas) in 1,4-dioxane (2.00 mL), and methanol (2.00 mL) was stirred for 1 h at room temperature. The resulting mixture was concentrated under reduced pressure to give a residue. The residue was purified by reverse flash chromatography (column, silica gel; mobile phase, acetonitrile in water; gradient, 10% to 50% in 10 min; detector, UV 254 nm), followed by prep-HPLC (Condition 7, Gradient 1) to afford (exo-)-N,1,5-trimethyl-N-[6-[4-(1H-pyrazol-4-yl)-1,3-benzothiazol-7-yl]pyridazin-3-yl]-8-azabicyclo[3.2.1]octan-3-amine (7.8 mg) as a solid. LCMS (ES, m/z): 446 [M+H]⁺. ¹H NMR (400 MHz, DMSO-d₆) δ 13.09 (s, 1H), 9.51 (s, 1H), 8.65 (s, 1H), 8.44 (s, 1H), 8.25 (d, J=9.9 Hz, 1H), 8.10 (d, J=8.1 Hz, 1H), 7.98 (d, J=8.0 Hz, 1H), 7.26 (d, J=9.8 Hz, 1H), 5.03 (s, 1H), 2.97 (s, 3H), 1.85 (d, J=7.4 Hz, 2H), 1.54 (t, J=10.0 Hz, 6H), 1.19 (s, 6H).

### Example 53: Synthesis of Compound 194

### Synthesis of Intermediate B150

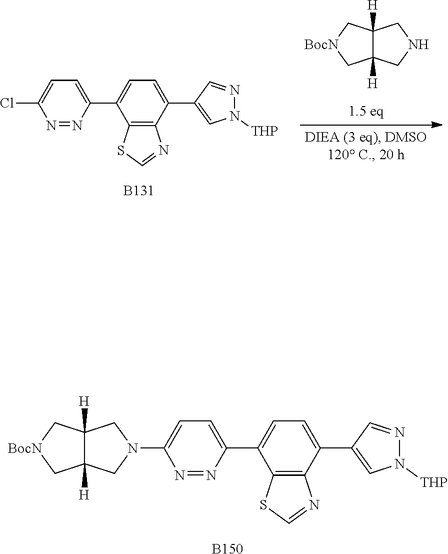

A mixture of 7-(6-chloropyridazin-3-yl)-4-[1-(oxan-2-yl)pyrazol-4-yl]-1,3-benzothiazole (100 mg, 0.246 mmol, 1.00 equiv), tert-butyl (exo)-hexahydro-1H-pyrrolo[3,4-c]pyrrole-2-carboxylate (78.43 mg, 0.369 mmol, 1.5 equiv) and DIEA (95.50 mg, 0.738 mmol, 3 equiv) in DMSO (1 mL) was stirred for 20 h at 120° C. The mixture was cooled to 25° C. The resulting mixture was poured into water (10 mL), then extracted with ethyl acetate (3×10 mL). The combined organic layers were washed with brine (2×10 mL), dried over anhydrous Na₂SO₄, and filtered. After filtration, the filtrate was concentrated under reduced pressure to afford tert-butyl (exo)-5-(6-{4-[1-(oxan-2-yl)pyrazol-4-yl]-1,3-benzothiazol-7-yl}pyridazin-3-yl)-hexahydropyrrolo[3,4-c]pyrrole-2-carboxylate (51 mg, 36.09%) as a solid. LCMS (ES, m/z): 574 [M+H]⁺.

### Synthesis of Compound 194

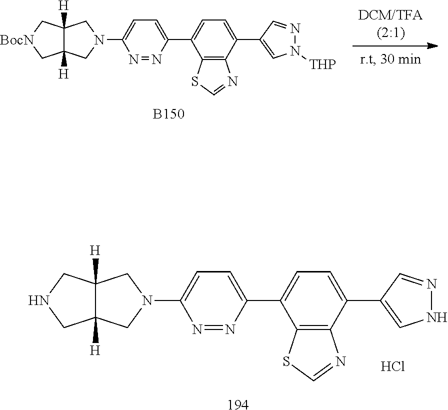

A solution of tert-butyl (exo)-5-(6-{4-[1-(oxan-2-yl)pyrazol-4-yl]-1,3-benzothiazol-7-yl}pyridazin-3-yl)-hexahydropyrrolo[3,4-c]pyrrole-2-carboxylate (51 mg, 0.087 mmol, 1.00 equiv) and TFA (1 mL) in DCM (2 mL) was stirred for 30 min at room temperature. The resulting mixture was concentrated under reduced pressure to give a residue. The residue was purified by Prep-HPLC (Condition 8, Gradient 3) to afford 7-{6-[(exo)-hexahydro-1H-pyrrolo[3,4-c]pyrrol-2-yl]pyridazin-3-yl}-4-(1H-pyrazol-4-yl)-1,3-benzothiazole hydrochloride (13.4 mg, 33.62%) as a solid. LCMS (ES, m/z):390 [M+H]⁺. ¹H NMR (400 MHz, DMSO-d₆) δ 9.81 (s, 1H), 9.61 (s, 1H), 9.50 (s, 1H), 8.71 (d, J=9.9 Hz, 1H), 8.60 (s, 2H), 8.31 (d, J=8.1 Hz, 1H), 8.07 (d, J=8.0 Hz, 1H), 7.73 (d, J=9.6 Hz, 1H), 3.87 (dt, J=11.7, 5.7 Hz, 4H), 3.46 (dt, J=12.3, 6.1 Hz, 2H), 3.28 (m, 4H).

### Example 54: Synthesis of Compound 198

### Synthesis of Intermediate B151

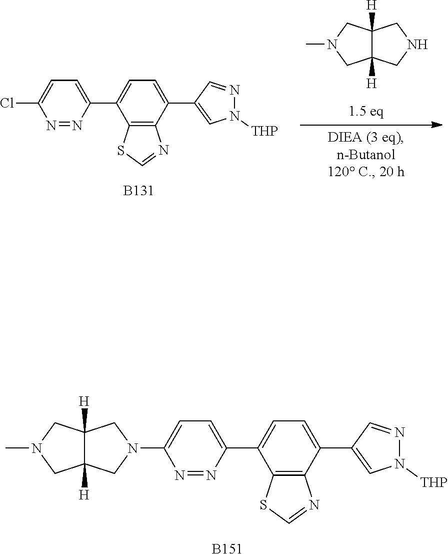

A solution of 7-(6-chloropyridazin-3-yl)-4-[1-(oxan-2-yl)pyrazol-4-yl]-1,3-benzothiazole (80 mg, 0.197 mmol, 1.00 equiv), (exo)-2-methyl-hexahydro-1H-pyrrolo[3,4-c]pyrrole (37.30 mg, 0.295 mmol, 1.50 equiv), and DIEA (76.40 mg, 0.591 mmol, 3.00 equiv) in n-Butanol (0.80 mL) was stirred for 20 h at 120° C. The resulting mixture was concentrated under vacuum, then poured into water (10 mL) and extracted with ethyl acetate (3×10 mL), dried over anhydrous Na₂SO₄, and filtered. After filtration, the filtrate was concentrated under reduced pressure to afford 7-{6-[(exo)-5-methyl-hexahydropyrrolo[3,4-c]pyrrol-2-yl]pyridazin-3-yl}-4-[1-(oxan-2-yl)pyrazol-4-yl]-1,3-benzothiazole (50 mg, 51.00%) as a solid. LCMS (ES, m/z): 488 [M+H]⁺.

### Synthesis of Compound 198

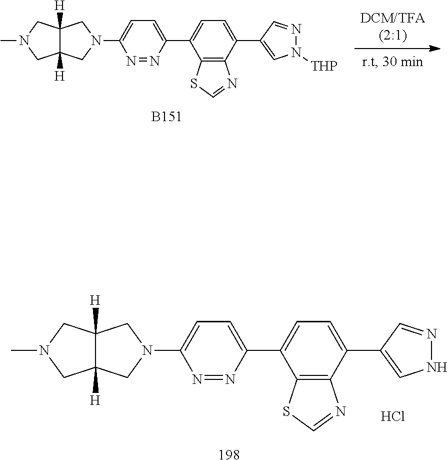

A solution of 7-{6-[(exo)-5-methyl-hexahydropyrrolo[3,4-c]pyrrol-2-yl]pyridazin-3-yl}-4-[1-(oxan-2-yl)pyrazol-4-yl]-1,3-benzothiazole (50 mg, 0.100 mmol, 1.00 equiv) and TFA (1 mL) in DCM (2 mL) was stirred for 30 min at room temperature. The resulting mixture was concentrated under reduced pressure to give a residue. The residue was purified by Prep-HPLC (Condition 8, Gradient 3) to afford 7-{6-[(exo)-5-methyl-hexahydropyrrolo[3,4-c]pyrrol-2-yl]pyridazin-3-yl}-4-(1H-pyrazol-4-yl)-1,3-benzothiazole hydrochloride (13.4 mg, 30.07%) as a solid. LCMS (ES, m/z):404 [M+H]⁺. ¹H NMR (400 MHz, Methanol-d₄) δ 9.45 (d, J=1.7 Hz, 1H), 8.83 (d, J=2.6 Hz, 2H), 8.76 (t, J=10.9 Hz, 1H), 8.27 (t, J=7.7 Hz, 1H), 8.11 (dd, J=8.1, 4.8 Hz, 1H), 7.92 (dd, J=14.7, 9.9 Hz, 1H), 4.16-4.04 (m, 5H), 3.85 (d, J=12.1 Hz, 1H), 3.68 (s, 1H), 3.62-3.48 (m, 2H), 3.08 (d, J=8.3 Hz, 1H), 3.01 (s, 3H).

### Example 55: Synthesis of Compound 205

### Synthesis of Intermediate B152

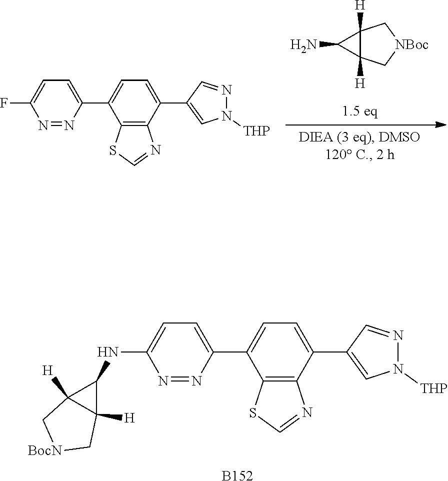

A mixture of 7-(6-fluoropyridazin-3-yl)-4-[1-(oxan-2-yl)pyrazol-4-yl]-1,3-benzothiazole (200.00 mg, 0.524 mmol, 1.00 equiv), tert-butyl (exo-)-6-amino-3-azabicyclo[3.1.0]hexane-3-carboxylate (155.94 mg, 0.787 mmol, 1.5 equiv), DIEA (203.30 mg, 1.573 mmol, 3.00 equiv), and DMSO (10.00 mL, 140.786 mmol, 268.50 equiv) was stirred overnight at 120° C. The reaction mixture was quenched with water at room temperature and extracted with ethyl acetate (3×10 mL). The combined organic layers were washed with brine (2×10 mL), dried over anhydrous Na₂SO₄, and filtered. After filtration, the filtrate was concentrated under reduced pressure to afford tert-butyl (exo-)-6-[(6-[4-[1-(oxan-2-yl)pyrazol-4-yl]-1,3-benzothiazol-7-yl]pyridazin-3-yl)amino]-3-azabicyclo[3.1.0]hexane-3-carboxylate (260 mg, 88.60%) as a solid. LCMS (ES, m/z): 560 [M+H]⁺.

### Synthesis of Intermediate B153

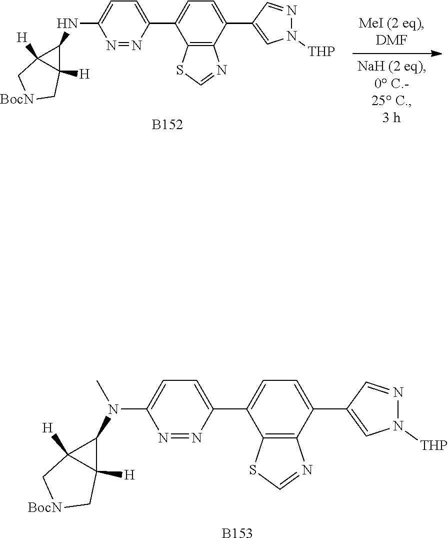

To a solution of tert-butyl (exo-)-6-[(6-[4-[1-(oxan-2-yl)pyrazol-4-yl]-1,3-benzothiazol-7-yl]pyridazin-3-yl)amino]-3-azabicyclo[3.1.0]hexane-3-carboxylate (10.00 mg, 0.018 mmol, 1.00 equiv) in DMF was added sodium hydride (60% in oil, 3 mg) at 0° C. The reaction mixture was stirred for 15 min. CH₃I (3.80 mg, 0.027 mmol, 1.5 equiv) was added and the reaction mixture was allowed to warm to room temperature and stirred for 2 h. The reaction mixture was quenched with water and extracted with DCM (3×25 mL). The resulting mixture was concentrated under reduced pressure to afford tert-butyl (exo-)-6-[methyl(6-[4-[1-(oxan-2-yl)pyrazol-4-yl]-1,3-benzothiazol-7-yl]pyridazin-3-yl)amino]-3-azabicyclo[3.1.0]hexane-3-carboxylate (200 mg, 83.03%) as a solid. LCMS (ES, m/z): 574 [M+H]⁺.

### Synthesis of Compound 205

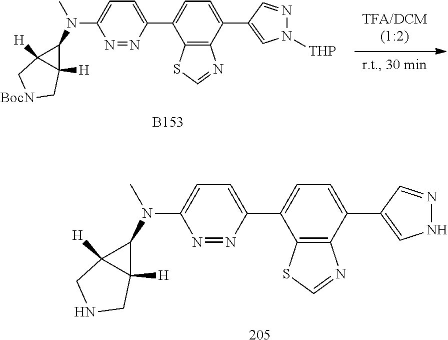

A mixture of tert-butyl (exo-)-6-[methyl(6-[4-[1-(oxan-2-yl)pyrazol-4-yl]-1,3-benzothiazol-7-yl] pyridazin-3-yl)amino]-3-azabicyclo[3.1.0]hexane-3-carboxylate (180.00 mg), TFA (1.00 mL), and DCM (5.00 mL) was stirred for 1 h at room temperature. The resulting mixture was concentrated under reduced pressure to give a residue. The residue was purified by reverse flash chromatography (column, silica gel; mobile phase, acetonitrile in water; gradient, 10% to 50% in 10 min; detector, UV 254 nm), followed by prep-HPLC (Condition 7, Gradient 1) to afford (exo-)-N-methyl-N-[6-[4-(1H-pyrazol-4-yl)-1,3-benzothiazol-7-yl]pyridazin-3-yl]-3-azabicyclo[3.1.0]hexan-6-amine (10 mg) as a solid. LCMS (ES, m/z): 390 [M+H]⁺. ¹H NMR (400 MHz, DMSO-d₆) δ 12.86 (brs, 1H), 9.56 (s, 1H), 8.49 (s, 2H), 8.24 (d, J=9.6 Hz, 1H), 8.05 (d, J=8.0 Hz, 1H), 7.96 (d, J=8.0 Hz, 1H), 7.35 (d, J=9.6 Hz, 1H), 3.3 (s, 3H), 3.14 (d, J=10.8 Hz, 2H), 2.85 (d, J=10.8 Hz, 2H), 2.565 (s, 1H), 1.78 (s, 2H).

### Example 56: Synthesis of Compound 235

### Synthesis of Compound 235

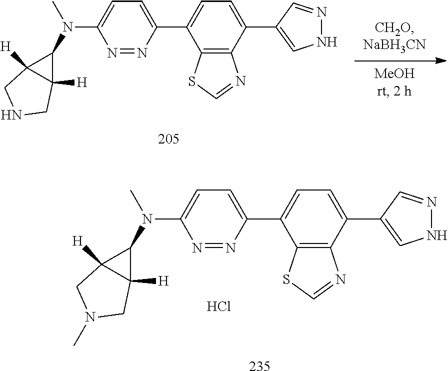

To a mixture of (exo)-N-methyl-N-{6-[4-(1H-pyrazol-4-yl)-1,3-benzothiazol-7-yl]pyridazin-3-yl}-3-azabicyclo[3.1.0]hexan-6-amine (80 mg, 0.205 mmol, 1.00 equiv), CH₂O (0.5 mL), and methanol (2 mL, 49.398 mmol, 240.49 equiv) was added NaBH₃CN (10 mg) in portions at room temperature. The resulting mixture was stirred for 2 h at room temperature, then concentrated under reduced pressure to give a residue. The residue was purified by reverse flash chromatography (column, silica gel; mobile phase, acetonitrile in water; gradient, 10% to 50% in 10 min; detector, UV 254 nm), followed by prep-HPLC (Condition 8, Gradient 1, Gradient 4) to afford (exo)-N,3-dimethyl-N-{6-[4-(1H-pyrazol-4-yl)-1,3-benzothiazol-7-yl]pyridazin-3-yl}-3-azabicyclo[3.1.0]hexan-6-amine hydrochloride (2 mg, 2.21%) as a solid. LCMS (ES, m/z): 440 [M+H]⁺. 1H NMR (400 MHz, DMSO-d₆) δ 10.68 (s, 1H), 9.55 (s, 1H), 8.58 (s, 2H), 8.44 (d, J=9.8 Hz, 1H), 8.20 (d, J=8.0 Hz, 1H), 8.03 (d, J=7.9 Hz, 1H), 7.50 (d, J=9.8 Hz, 1H), 3.89 (dd, J=11.5, 5.1 Hz, 2H), 3.41 (t, J=9.2 Hz, 2H), 3.3 (m, 1H), 3.27 (s, 3H), 2.82 (d, J=4.5 Hz, 3H), 2.29 (s, 2H).

### Example 57: Synthesis of Compound 200

### Synthesis of Intermediate B154

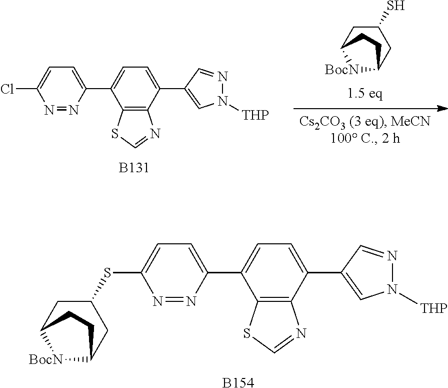

A mixture of 7-(6-chloropyridazin-3-yl)-4-[1-(oxan-2-yl)pyrazol-4-yl]-1,3-benzothiazole (80 mg, 0.197 mmol, 1.00 equiv), tert-butyl (exo)-3-sulfanyl-8-azabicyclo[3.2.1]octane-8-carboxylate (71.93 mg, 0.295 mmol, 1.5 equiv), and Cs₂CO₃ (76.40 mg, 0.591 mmol, 3 equiv) in acetonitrile (0.8 mL) was stirred for 2 h at 100° C. The reaction mixture was cooled to 25° C., then poured into water (10 mL) and extracted with ethyl acetate (3×10 mL). The combined organic layers were washed with brine (2×10 mL), dried over anhydrous Na₂SO₄, and filtered. After filtration, the filtrate was concentrated under reduced pressure to afford tert-butyl (exo)-3-[(6-{4-[1-(oxan-2-yl)pyrazol-4-yl]-1,3-benzothiazol-7-yl}pyridazin-3-yl)sulfanyl]-8-azabicyclo[3.2.1]octane-8-carboxylate (60 mg, 49.34%) as a solid. LCMS (ES, m/z): 605 [M+H]⁺.

### Synthesis of Compound 200

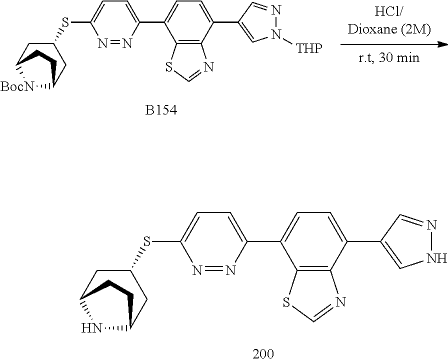

A mixture of tert-butyl (exo)-3-[(6-{4-[1-(oxan-2-yl)pyrazol-4-yl]-1,3-benzothiazol-7-yl}pyridazin-3-yl)sulfanyl]-8-azabicyclo[3.2.1]octane-8-carboxylate (50 mg, 0.081 mmol, 1.00 equiv) and HCl (gas) in 1,4-dioxane (0.5 mL) was stirred for 30 min at room temperature. The resulting mixture was concentrated under reduced pressure to give a residue. The residue was purified by Prep-HPLC (Condition 8, Gradient 5) to afford 7-{6-[(exo)-8-azabicyclo[3.2.1]octan-3-ylsulfanyl]pyridazin-3-yl}-4-(1H-pyrazol-4-yl)-1,3-benzothiazole (14.2 mg, 40.76%) as a solid. LCMS (ES, m/z): 421 [M+H]⁺. ¹H NMR (400 MHz, DMSO-d₆) δ 9.56 (s, 1H), 9.20 (s, 1H), 9.06 (s, 1H), 8.61 (s, 2H), 8.48 (d, J=9.3 Hz, 1H), 8.31 (d, J=8.1 Hz, 1H), 8.07 (d, J=8.0 Hz, 1H), 7.83 (d, J=9.2 Hz, 1H), 4.66 (t, J=7.0 Hz, 1H), 4.06 (q, J=3.6 Hz, 2H), 2.75 (ddd, J=16.1, 7.3, 3.4 Hz, 2H), 2.26 (t, J=7.1 Hz, 2H), 2.17-2.05 (m, 4H).

### Example 58: Synthesis of Compound 242

### Synthesis of Intermediate B155

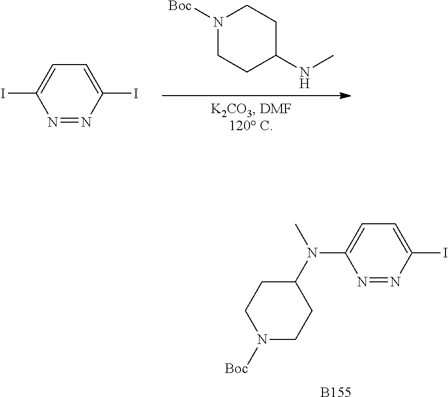

A mixture of 3,6-diiodopyridazine (1.00 g, 3.013 mmol, 1.00 equiv), tert-butyl 4-(methylamino)piperidine-1-carboxylate (0.77 g, 3.616 mmol, 1.2 equiv), and K₂CO₃ (1.25 g, 9.039 mmol, 3 equiv) in DMF (10 mL) was stirred for 16 h at 120° C. The reaction mixture was cooled to room temperature, and poured into water. The aqueous layer was extracted with ethyl acetate (3×100 mL). The combined organic layers were dried over anhydrous Na₂SO₄, filtered, and the filtrate was concentrated under reduced pressure to give a residue. The residue was purified by silica gel column chromatography, eluted with EA/PE (0-100%) to afford tert-butyl 4-[(6-iodopyridazin-3-yl)(methyl)amino]piperidine-1-carboxylate (500 mg, 39.67%) as a solid. LCMS (ES, m/z): 419 [M+H]⁺.

### Synthesis of Intermediate B156

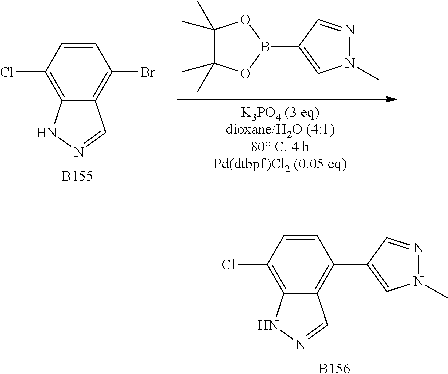

To a solution of 4-bromo-7-chloro-1H-indazole (3.10 g, 13.392 mmol, 1 equiv) and 1-methyl-4-(4,4,5,5-tetramethyl-1,3,2-dioxaborolan-2-yl)pyrazole (3.34 g, 16.070 mmol, 1.2 equiv) in dioxane (32 mL) and H₂O (8 mL) was added Pd(DtBPF)Cl₂ (0.48 g, 0.738 mmol, 0.0551 equiv) and K₃PO₄ (8.53 g, 40.176 mmol, 3 equiv). The reaction mixture was stirred for 4 h at 80° C. under a nitrogen atmosphere, then cooled to room temperature. The resulting mixture was extracted with ethyl acetate (3×20 mL). The combined organic layers were washed with water (1×60 mL) and brine (1×60 mL), dried over anhydrous Na₂SO₄, and filtered. After filtration, the filtrate was concentrated under reduced pressure to give a residue. The residue was purified by silica gel column chromatography, eluted with DCM/MeOH (20/1) to afford 7-chloro-4-(1-methylpyrazol-4-yl)-1H-indazole (2.1 g, 67.40%) as a solid. LCMS (ES, m/z): 233 [M+H]⁺.

### Synthesis of Intermediate B157

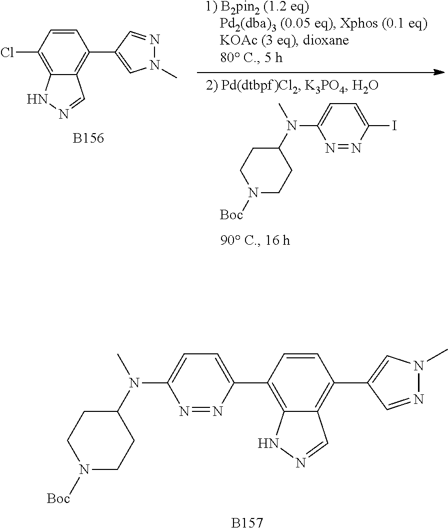

To a mixture of 7-chloro-4-(1-methylpyrazol-4-yl)-1H-indazole (50 mg, 0.215 mmol, 1 equiv) and bis(pinacolato)diboron (65 mg, 0.258 mmol, 1.2 equiv) in dioxane (1 mL) was added Xphos (10 mg, 0.022 mmol, 0.1 equiv) and KOAc (63 mg, 0.645 mmol, 3 equiv). The reaction mixture was stirred for 16 h at 80° C. under a nitrogen atmosphere, then cooled to room temperature. To the reaction mixture was added tert-butyl 4-[(6-iodopyridazin-3-yl)(methyl)amino]piperidine-1-carboxylate (64 mg, 0.154 mmol, 1 equiv), Pd(dtbpf)Cl₂ (10 mg, 0.015 mmol, 0.1 equiv), K₃PO₄ (98 mg, 0.462 mmol, 3 equiv), and water (40 μL) at room temperature. The resulting mixture was stirred for an additional 1 h at 80° C., then cooled to room temperature and extracted with ethyl acetate (3×20 mL). The combined organic layers were washed with water (1×60 mL) and brine (1×60 mL), dried over anhydrous Na₂SO₄, and filtered. After filtration, the filtrate was concentrated under reduced pressure to give a residue. The residue was purified by silica gel column chromatography, eluted with DCM/MeOH (20/1) to afford tert-butyl 4-[methyl({6-[4-(1-methylpyrazol-4-yl)-1H-indazol-7-yl]pyridazin-3-yl})amino]piperidine-1-carboxylate (60 mg, 79.62%) as a solid. LCMS (ES, m/z): 489 [M+H]⁺.

### Synthesis of Compound 242

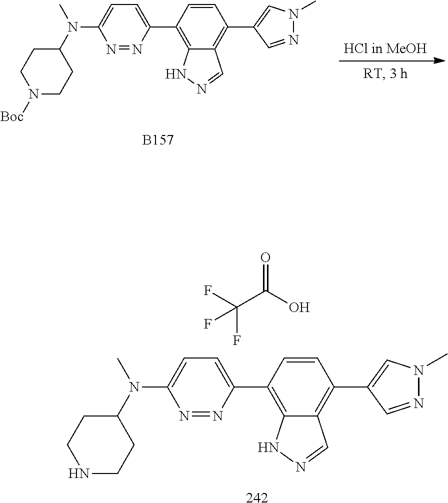

A mixture of tert-butyl 4-[methyl({6-[4-(1-methylpyrazol-4-yl)-1H-indazol-7-yl]pyridazin-3-yl}) amino]piperidine-1-carboxylate (60 mg) and HCl (g) in MeOH (1 mL) was stirred for 3 h at room temperature. The resulting mixture was concentrated under reduced pressure to give a residue. The residue was purified by prep-HPLC (Condition 9, Gradient 1) to afford N-methyl-6-[4-(1-methylpyrazol-4-yl)-1H-indazol-7-yl]-N-(piperidin-4-yl)pyridazin-3-amine;trifluoroacetic acid (23 mg, 37.27%) as a solid. LCMS (ES, m/z): 389 [M+H]⁺. ¹H NMR (400 MHz, DMSO-d₆) δ 13.04 (s, 1H), 8.61 (s, 1H), 8.50 (d, J=9.3 Hz, 2H), 8.29 (d, J=9.8 Hz, 2H), 8.13 (s, 1H), 7.97 (d, J=7.7 Hz, 1H), 7.49-7.42 (m, 2H), 4.85 (s, 1H), 3.96 (s, 3H), 3.46 (d, J=12.4 Hz, 2H), 3.11 (q, J=11.9 Hz, 2H), 3.04 (s, 3H), 2.07-1.97 (m, 2H), 1.95-1.85 (m, 2H).

### Example 59: Synthesis of Compound 243

### Synthesis of Compound 243

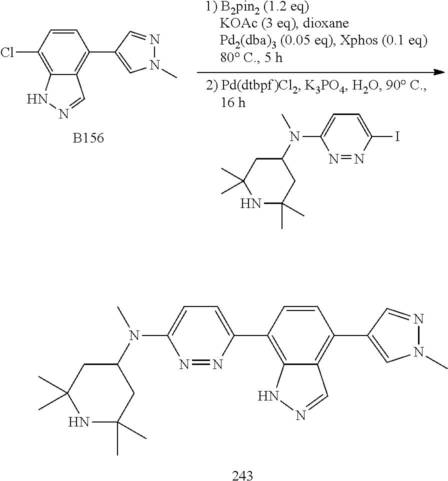

To a mixture of 7-chloro-4-(1-methylpyrazol-4-yl)-1H-indazole (50 mg, 0.215 mmol, 1 equiv) and bis(pinacolato)diboron (65 mg, 0.258 mmol, 1.2 equiv) in dioxane (1 mL) was added Xphos (10 mg, 0.022 mmol, 0.1 equiv) and KOAc (63 mg, 0.645 mmol, 3 equiv). The reaction mixture was stirred for 16 h at 80° C. under a nitrogen atmosphere, then cooled to room temperature. To the reaction mixture was added 6-iodo-N-methyl-N-(2,2,6,6-tetramethylpiperidin-4-yl)pyridazin-3-amine (58 mg, 0.154 mmol, 1 equiv), Pd(dtbpf)Cl₂ (10 mg, 0.015 mmol, 0.1 equiv), K₃PO₄ (98 mg, 0.462 mmol, 3 equiv) and H₂O (40 μL). The resulting mixture was stirred for additional 1 h at 80° C., then cooled to room temperature. The resulting mixture was extracted with ethyl acetate (3×20 mL). The combined organic layers were washed with water (1×60 mL) and brine (1×60 mL), dried over anhydrous Na₂SO₄, and filtered. After filtration, the filtrate was concentrated under reduced pressure to give a residue. The residue was purified by Prep-HPLC (Condition 10 Gradient 1) to afford N-methyl-6-[4-(1-methylpyrazol-4-yl)-1H-indazol-7-yl]-N-(2,2,6,6-tetramethylpiperidin-4-yl)pyridazin-3-amine; trifluoroacetic acid (10.2 mg, 11.84%) as a solid. LCMS (ES, m/z): 445 [M+H]⁺. ¹H NMR (400 MHz, DMSO-d₆) δ 13.10 (Br, 1H), 8.75 (d, J=12.2 Hz, 1H), 8.50 (d, J=11.4 Hz, 2H), 8.27 (d, J=9.8 Hz, 1H), 8.12 (s, 1H), 7.95 (d, J=7.7 Hz, 1H), 7.85 (d, J=12.1 Hz, 1H), 7.45 (dd, J=8.5, 4.3 Hz, 2H), 5.24 (s, 1H), 3.96 (s, 3H), 3.04 (s, 3H), 1.97-1.84 (m, 4H), 1.57 (s, 6H), 1.45 (s, 6H).

### Example 60: Synthesis of Compound 232

### Synthesis of Compound 232

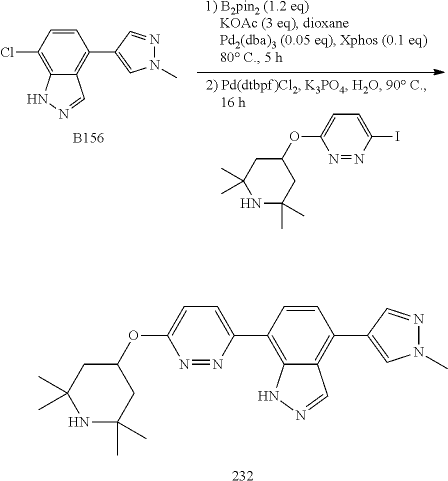

To a mixture of 7-chloro-4-(1-methylpyrazol-4-yl)-1H-indazole (50 mg, 0.215 mmol, 1 equiv) and bis(pinacolato)diboron (65 mg, 0.258 mmol, 1.2 equiv) in dioxane (1 mL) was added Xphos (10 mg, 0.022 mmol, 0.1 equiv) and KOAc (63 mg, 0.645 mmol, 3 equiv). The reaction mixture was stirred for 16 h at 80° C. under a nitrogen atmosphere, then cooled to room temperature. To the reaction mixture was added 3-iodo-6-[(2,2,6,6-tetramethylpiperidin-4-yl)oxy]pyridazine (55 mg, 0.154 mmol, 1 equiv), Pd(dtbpf)Cl₂ (10 mg, 0.015 mmol, 0.1 equiv), K₃PO₄ (98 mg, 0.462 mmol, 3 equiv) and H₂O (40 μL) at room temperature. The resulting mixture was stirred for an additional 1 h at 80° C., then cooled to room temperature and extracted with ethyl acetate (3×20 mL). The combined organic layers were washed with water (1×60 mL) and brine (1×60 mL), dried over anhydrous Na₂SO₄, and filtered. After filtration, the filtrate was concentrated under reduced pressure to give a residue. The residue was purified by prep-HPLC (Condition 11, Gradient 1) to afford 4-(1-methylpyrazol-4-yl)-7-{6-[(2,2,6,6-tetramethylpiperidin-4-yl)oxy]pyridazin-3-yl}-1H-indazole (12.9 mg, 19.38%) as a solid. LCMS (ES, m/z): 432 [M+H]⁺. 1H NMR (400 MHz, DMSO-d₆) δ 13.17 (s, 1H), 8.52 (d, J=8.1 Hz, 2H), 8.40 (d, J=9.4 Hz, 1H), 8.14 (s, 1H), 8.00 (d, J=7.7 Hz, 1H), 7.48 (d, J=7.7 Hz, 1H), 7.32 (d, J=9.3 Hz, 1H), 5.79 (tt, J=11.3, 4.2 Hz, 1H), 3.96 (s, 3H), 2.13 (dd, J=12.0, 4.1 Hz, 2H), 1.36-1.18 (m, 9H), 1.13 (s, 6H).

### Example 61: Synthesis of Compound 174-179, 233, and 234

### Synthesis of Intermediate B158

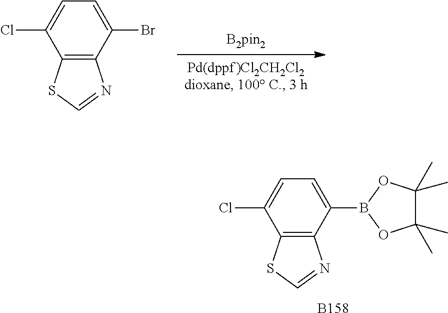

To a mixture of 4-bromo-7-chloro-1,3-benzothiazole (5 g, 20.11 mmol, 1.0 equiv) and bis(pinacolato)diboron (6.13 g, 24.14 mmol, 1.2 equiv) in 1,4-dioxane (50 mL, 567.5 mmol, 28.2 equiv) was added KOAc (5.92 g, 60.35 mmol, 3 equiv) and Pd(dppf)Cl₂.CH₂Cl₂ (0.82 g, 1.006 mmol, 0.05 equiv). The reaction mixture was stirred for 3 h at 100° C. under a nitrogen atmosphere, then filtered, the filter cake was washed with methanol (3×30 ml). The filtrate was concentrated to afford 7-chloro-4-(4,4,5,5-tetramethyl-1,3,2-dioxaborolan-2-yl)-1,3-benzothiazole (5 g, 84.1%) as an oil. LCMS (ES, m/z): 296 [M+H]⁺.

### Synthesis of Intermediate B159

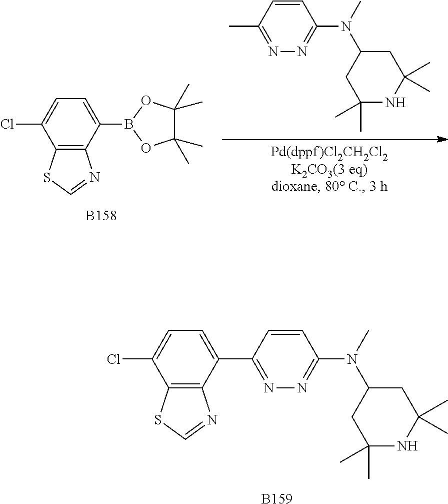

To a solution of 7-chloro-4-(4,4,5,5-tetramethyl-1,3,2-dioxaborolan-2-yl)-1,3-benzothiazole (4.5 g, 15.24 mmol, 1.0 equiv) and 6-iodo-N-methyl-N-(2,2,6,6-tetramethylpiperidin-4-yl)pyridazin-3-amine (6.84 g, 18.26 mmol, 1.2 equiv) in 1,4-dioxane (40 mL, 454.0 mmol, 29.82 equiv) and H₂O (10 mL, 555.08 mmol, 36.46 equiv) was added K₂CO₃ (6.31 g, 45.67 mmol, 3 equiv) and Pd(dppf)Cl₂.CH₂Cl₂ (0.62 g, 0.761 mmol, 0.05 equiv). The reaction mixture was stirred for 3 h at 80° C. under a nitrogen atmosphere, then concentrated under reduced pressure to give a residue. The residue was purified by Prep-TLC/silica gel column chromatography, eluted with (DCM/MeOH=9/1) to afford 6-(7-chloro-1,3-benzothiazol-4-yl)-N-methyl-N-(2,2,6,6-tetramethylpiperidin-4-yl)pyridazin-3-amine (2.1 g, 33.1%) as a solid. LCMS (ES, m/z): 416 [M+H]⁺.

### Synthesis of Compound 175

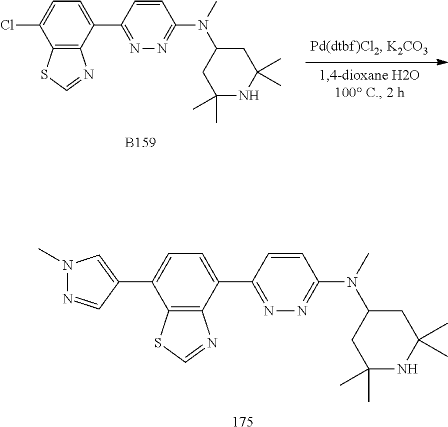

To a solution of 6-(7-chloro-1,3-benzothiazol-4-yl)-N-methyl-N-(2,2,6,6-tetramethylpiperidin-4-yl)pyridazin-3-amine (70 mg, 0.168 mmol, 1.00 equiv) and 1-methyl-4-(4,4,5,5-tetramethyl-1,3,2-dioxaborolan-2-yl)pyrazole (38.51 mg, 0.185 mmol, 1.1 equiv) in 1,4-dioxane (2 mL) and H₂O (0.5 mL) was added K₂CO₃ (69.77 mg, 0.504 mmol, 3 equiv) and Pd(dtbpf)Cl₂ (11.1 mg, 0.017 mmol, 0.1 equiv). The reaction mixture was stirred for 2 h at 100° C. under a nitrogen atmosphere, then concentrated under reduced pressure to give a residue. The residue was purified by prep-HPLC (Condition 4, Gradient 8) to afford N-methyl-6-[7-(1-methylpyrazol-4-yl)-1,3-benzothiazol-4-yl]-N-(2,2,6,6-tetramethylpiperidin-4-yl)pyridazin-3-amine (7.2 mg, 9.27%) as a solid.

Compounds 174, 176-179, 233 and 234 were prepared according to the same procedure outlined in this Example 61 and generalized by Scheme C. Table 3 below provides intermediates used in these procedures and final compound characterization data.

TABLE 3
Intermediates and characterization data for compounds prepared according to Example 61 protocol and general Scheme C

| | | LCMS | |
|---|---|---|---|
| | | (ESI, m/z) | ¹H NMR (400 MHz, DMSO- |
| Compound Structure | B-LG¹ | [M + H]⁺ | d₆ ) δ |
| 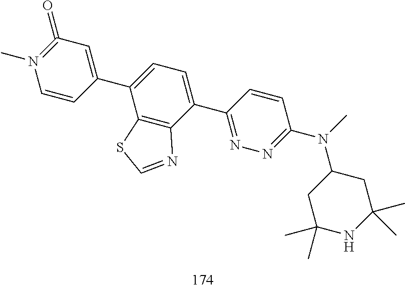 | 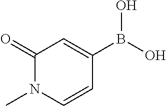 | 489 | 9.57 (s, 1H), 8.46 (d, J = 9.7 Hz, 1H), 8.34 (d, J = 7.9 Hz, 1H), 7.91 (d, J = 7.0 Hz, 1H), 7.80 (d, J = 8.0 Hz, 1H), 7.21 (d, J = 9.8 Hz, 1H), 6.77 (d, J = 2.1 Hz, 1H), 6.63 (dd, J = 7.0, 2.1 Hz, 1H), 5.15 (s, 1H), 3.52 (s, 3H), 2.98 (s, 3H), 1.56 (dd, J = 12.0, 3.6 Hz, 2H), 1.46 (t, J = 12.1 Hz, 2H), 1.28 (s, 6H), 1.11 (s, 6H) |
| 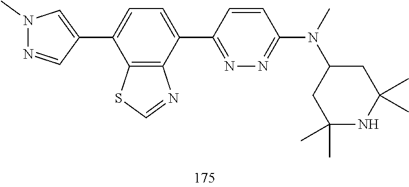 | 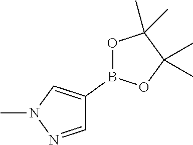 | 462 | 9.57 (s, 1H), 8.46 (d, J = 9.6 Hz, 1H), 8.36 (d, J = 0.9 Hz, 1H), 8.27 (d, J = 7.9 Hz, 1H), 8.07 (d, J = 0.9 Hz, 1H), 7.85 (d, J = 7.9 Hz, 1H), 7.19 (d, J = 9.7 Hz, 1H), 5.15 (s, 1H), 3.98 (s, 3H), 2.97 (s, 3H), 1.55 (dd, J = 12.0, 3.6 Hz, 2H), 1.46 (t, J = 12.1 Hz, 2H), 1.28 (s, 6H), 1.11 (s, 6H) |
| 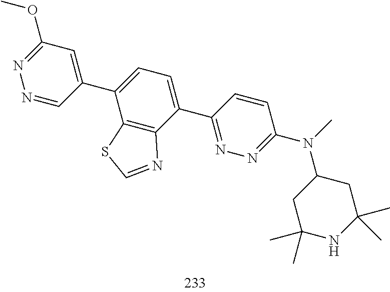 | 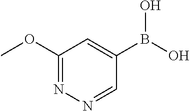 | 489 | 9.61 (s, 1H), 9.36 (d, J = 1.9 Hz, 1H), 8.47 (d, J = 9.6 Hz, 1H), 8.39 (d, J = 7.9 Hz, 1H), 7.95 (d, J = 8.0 Hz, 1H), 7.61 (d, J = 1.9 Hz, 1H), 7.21 (d, J = 9.7 Hz, 1H), 5.16 (s, 1H), 4.14 (s, 3H), 2.98 (s, 3H), 1.55 (dd, J = 12.1, 3.7 Hz, 2H), 1.47 (d, J = 12.1 Hz, 2H), 1.27 (s, 6H), 1.24 (s, 1H), 1.10 (s, 6H) |
| 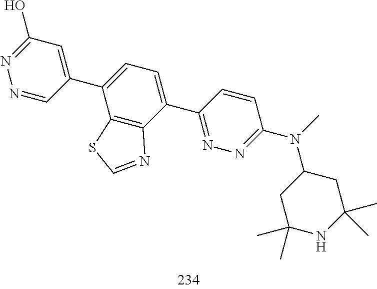 [SMILES: CN(c1ccc(-c2ccc(-c3cnnc(O)c3)c3scnc23)nn1)C1CC(C)(C)NC(C)(C)C1] | 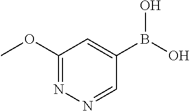 | 475 | 13.2-13.5 (s, 1H), 9.61 (s, 1H), 8.52 (d, J = 1.9 Hz, 1H), 8.45 (d, J = 9.6 Hz, 1H), 8.39 (d, J = 7.9 Hz, 1H), 7.95 (d, J = 8.0 Hz, 1H), 7.21 (d, J = 1.9 Hz, 2H), 5.16 (s, 1H), 2.98 (s, 3H), 1.55 (dd, J = 12.1, 3.7 Hz, 2H), 1.47 (d, J = 12.1 Hz, 2H), 1.27 (s, 6H), 1.10 (s, 6H) |
| 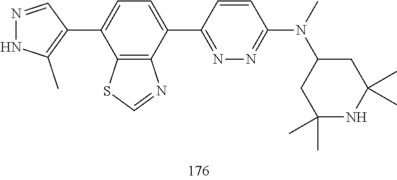 [SMILES: Cc1[nH]ncc1-c1ccc(-c2ccc(N(C)C3CC(C)(C)NC(C)(C)C3)nn2)c2ncsc12] | 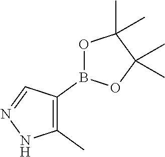 | 462 | 12.94 (s, 1H), 9.51 (s, 1H), 8.45 (d, J = 9.7 Hz, 1H), 8.26 (d, J = 7.8 Hz, 1H), 7.89 (s, 1H), 7.57 (d, J = 7.8 Hz, 1H), 7.19 (d, J = 9.7 Hz, 1H), 5.13 (s, 1H), 2.97 (s, 3H), 2.36 (s, 3H), 1.56 (dd, J = 12.1, 3.6 Hz, 2H), 1.46 (t, J = 12.1 Hz, 2H), 1.28 (s, 6H), 1.11 (s, 6H) |
| 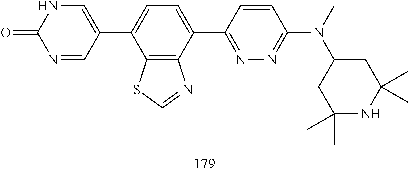 [SMILES: CN(c1ccc(-c2ccc(-c3cnc(=O)[nH]c3)c3scnc23)nn1)C1CC(C)(C)NC(C)(C)C1] | 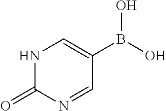 | 475 | 9.51 (s, 1H), 8.64 (s, 2H), 8.46 (d, J = 9.6 Hz, 1H), 8.33 (d, J = 7.9 Hz, 1H), 7.70 (d, J = 7.8 Hz, 1H), 7.16 (d, J = 9.6 Hz, 1H), 5.20-5.05 (m, 1H), 3.01 (s, 3H), 1.61 (dd, J = 12.2, 3.6 Hz, 2H), 1.49 (t, J = 12.2 Hz, 2H), 1.31 (s, 6H), 1.14 (s, 6H) |
| 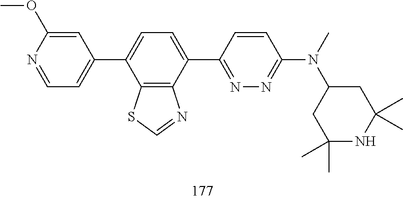 | 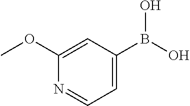 | 489 | 9.58 (d, J = 3.1 Hz, 1H), 8.47 (d, J = 9.7 Hz, 1H), 8.40-8.33 (m, 2H), 7.84 (d, J = 7.9 Hz, 1H), 7.41 (dd, J = 5.4, 1.6 Hz, 1H), 7.24-7.17 (m, 2H), 5.15 (s, 1H), 3.96 (s, 3H), 2.97 (d, J = 6.6 Hz, 3H), 1.56 (dd, J = 12.1, 3.7 Hz, 2H), 1.46 (t, J = 12.1 Hz, 2H), 1.27 (d, J = 4.2 Hz, 6H), 1.10 (d, J = 2.8 Hz, 6H) |
| 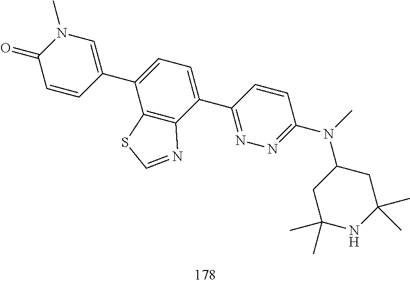 [SMILES: CN(c1ccc(-c2ccc(-c3ccc(=O)n(C)c3)c3scnc23)nn1)C1CC(C)(C)NC(C)(C)C1] | 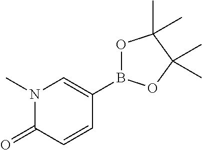 | 489 | 9.56 (s, 1H), 8.46 (d, J = 9.7 Hz, 1H), 8.31 (d, J = 7.9 Hz, 1H), 8.23 (d, J = 2.7 Hz, 1H), 7.89 (dd, J = 9.4, 2.8 Hz, 1H), 7.66 (d, J = 7.9 Hz, 1H), 7.20 (d, J = 9.7 Hz, 1H), 6.60 (d, J = 9.4 Hz, 1H), 5.16 (s, 1H), 3.56 (s, 3H), 2.97 (s, 3H), 1.55 (dd, J = 12.0, 3.6 Hz, 2H), 1.48 (d, J = 12.2 Hz, 2H), 1.28 (s, 6H), 1.11 (s, 6H) |

### Example 62: Synthesis of Compound 283

### Synthesis of Intermediate B160

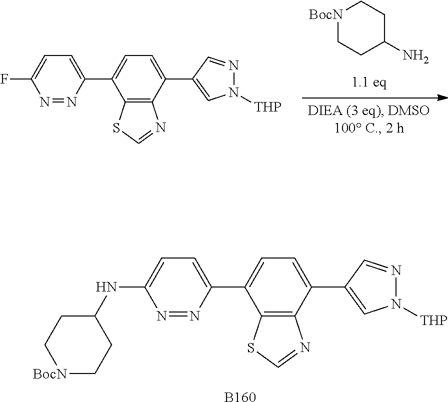

A mixture of 7-(6-fluoropyridazin-3-yl)-4-[1-(oxan-2-yl) pyrazol-4-yl]-1,3-benzothiazole (730.00 mg, 1.9 mmol, 1.00 equiv), tert-butyl 4-aminopiperidine-1-carboxylate (421.64 mg, 2.1 mmol, 1.10 equiv), and DIEA (742.06 mg, 5.7 mmol, 3.00 equiv) in DMSO (22.0 mL) was stirred for 2 h at 100° C. The reaction mixture was cooled to room temperature, then diluted with water (20.0 mL). A precipitate formed, and the solid was collected by filtration and washed with water (2×20.0 mL) to afford tert-butyl 4-[(6-{4-[1-(oxan-2-yl) pyrazol-4-yl]-1,3-benzothiazol-7-yl} pyridazin-3-yl) amino] piperidine-1-carboxylate (750.00 mg, 69.7%) as a solid. LCMS (ES, m/z): 562 [M+H]⁺.

### Synthesis of Intermediate B161

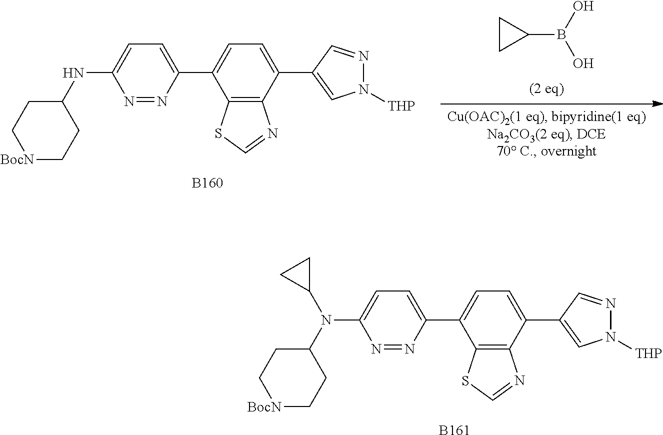

A mixture of tert-butyl 4-[(6-{4-[1-(oxan-2-yl) pyrazol-4-yl]-1,3-benzothiazol-7-yl} pyridazin-3-yl) amino] piperidine-1-carboxylate (700.00 mg, 1.2 mmol, 1.00 equiv), cyclopropylboronic acid (214.10 mg, 2.4 mmol, 2.00 equiv), bipyridyl (194.64 mg, 1.2 mmol, 1.00 equiv), Cu(OAc)₂ (226.35 mg, 1.2 mmol, 1.00 equiv), and Na₂CO₃ (264.16 mg, 2.4 mmol, 2.00 equiv) in DCE (35.0 mL) was stirred over night at 70° C. under oxygen atmosphere. The reaction mixture was cooled to room temperature, diluted with water (50.0 mL), and extracted with EA (2×50.0 mL). The combined organic layers were dried over anhydrous Na₂SO₄ and filtered. After filtration, the filtrate was concentrated under reduced pressure to give a residue. The residue was purified by silica gel column chromatography, eluted with PE/EA (1:2) to afford tert-butyl 4-[cyclopropyl(6-{4-[1-(oxan-2-yl) pyrazol-4-yl]-1,3-benzothiazol-7-yl} pyridazin-3-yl) amino] piperidine-1-carboxylate (150.00 mg, 20.0%) as a solid. LCMS (ES, m/z): 602 [M+H]⁺.

### Synthesis of Compound 283

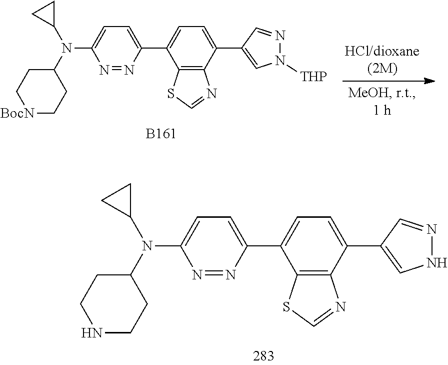

A solution of tert-butyl 4-[cyclopropyl(6-{4-[1-(oxan-2-yl) pyrazol-4-yl]-1,3-benzothiazol-7-yl}pyridazin-3-yl) amino] piperidine-1-carboxylate (135 mg, 0.2 mmol, 1.00 equiv) and HCl (gas) in 1,4-dioxane (1.35 mL) and methanol (4.05 mL) was stirred for 1 h at room temperature. The resulting mixture was concentrated under vacuum to give a residue. The residue was purified by prep-HPLC (Condition 7, Gradient 3) to afford N-cyclopropyl-N-(piperidin-4-yl)-6-[4-(1H-pyrazol-4-yl)-1,3-benzothiazol-7-yl] pyridazin-3-amine (41.70 mg, 43.7%) as a solid. LCMS (ES, m/z): 418 [M+H]⁺. ¹H NMR (400 MHz, DMSO-d₆) δ 13.10 (s, 1H), 9.52 (s, 1H), 8.55 (s, 2H), 8.31 (d, J=9.6 Hz, 1H), 8.12 (d, J=8.1 Hz, 1H), 8.00 (d, J=8.0 Hz, 1H), 7.50 (d, J=9.7 Hz, 1H), 4.57 (tt, J=12.1, 3.9 Hz, 1H), 3.05 (d, J=11.9 Hz, 2H), 2.68-2.56 (m, 3H), 1.98 (qd, J=12.1, 4.0 Hz, 2H), 1.82 (d, J=11.8 Hz, 2H), 1.01 (dd, J=7.1, 5.0 Hz, 2H), 0.68 (p, J=4.6 Hz, 2H).

### Example 63: Synthesis of Compound 284

### Synthesis of Intermediate B162

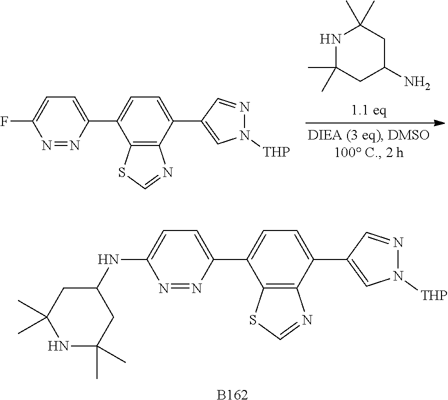

A mixture of 7-(6-fluoropyridazin-3-yl)-4-[1-(oxan-2-yl) pyrazol-4-yl]-1,3-benzothiazole (300.00 mg, 0.7 mmol, 1.00 equiv), 2,2,6,6-tetramethylpiperidin-4-amine (135.20 mg, 0.8 mmol, 1.10 equiv) and DIEA (304.95 mg, 2.3 mmol, 3.00 equiv) in DMSO (9.0 mL) was stirred for 2 h at 100° C. The reaction mixture was cooled to room temperature, then diluted with water (20.0 mL). A precipitate formed, and the solid was collected by filtration and washed with water (2×20.0 mL). The resulting mixture was concentrated under vacuum to afford 6-{4-[1-(oxan-2-yl) pyrazol-4-yl]-1,3-benzothiazol-7-yl}-N-(2,2,6,6-tetramethylpiperidin-4-yl) pyridazin-3-amine (395.00 mg, 97.0%) as a solid. LCMS (ES, m/z): 518 [M+H]⁺.

### Synthesis of Intermediate B163

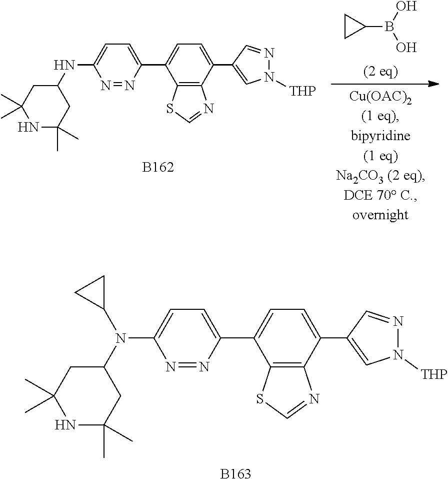

A mixture of 6-{4-[1-(oxan-2-yl) pyrazol-4-yl]-1,3-benzothiazol-7-yl}-N-(2,2,6,6-tetramethylpiperidin-4-yl) pyridazin-3-amine (520.00 mg, 1.0 mmol, 1.00 equiv), cyclopropylboronic acid (172.56 mg, 2.0 mmol, 2.00 equiv), bipyridyl (156.88 mg, 1.0 mmol, 1.00 equiv), Cu(OAc)₂ (182.44 mg, 1.0 mmol, 1.00 equiv) and Na₂CO₃ (212.92 mg, 2.0 mmol, 2.00 equiv) in DCE (26.0 mL) was stirred overnight at 70° C. under oxygen atmosphere. The reaction mixture was cooled to room temperature, diluted with water (50.0 mL), and extracted with ethyl acetate (2×50.0 mL). The combined organic layers were dried over anhydrous Na₂SO₄ and filtered. After filtration, the filtrate was concentrated under reduced pressure to give a residue. The residue was purified by silica gel column chromatography, eluted with PE/EA (1:2) to afford N-cyclopropyl-6-{4-[1-(oxan-2-yl) pyrazol-4-yl]-1,3-benzothiazol-7-yl}-N-(2,2,6,6-tetramethylpiperidin-4-yl) pyridazin-3-amine (158.00 mg, 28.2%) as a solid. LCMS (ES, m/z): 558 [M+H]⁺.

### Synthesis of Compound 284

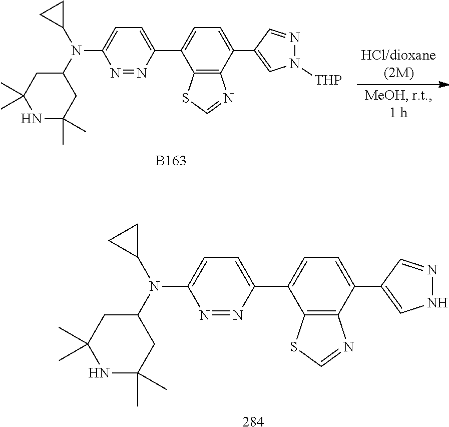

A mixture of N-cyclopropyl-6-{4-[1-(oxan-2-yl) pyrazol-4-yl]-1,3-benzothiazol-7-yl}-N-(2,2,6,6-tetramethylpiperidin-4-yl) pyridazin-3-amine (150.00 mg, 0.2 mmol, 1.00 equiv) and HCl (gas) in 1,4-dioxane (1.5 mL) and methanol (1.5 mL) was stirred for 1 h at room temperature. The resulting mixture was concentrated under vacuum to give a residue. The residue was purified by Chiral-Prep-HPLC (Condition 1, Gradient 1) to afford N-cyclopropyl-6-[4-(1H-pyrazol-4-yl)-1,3-benzothiazol-7-yl]-N-(2,2,6,6-tetramethylpiperidin-4-yl) pyridazin-3-amine (14.50 mg, 11.3%) as a solid. LCMS (ES, m/z): 474 [M+H]⁺. ¹H NMR (400 MHz, DMSO-d₆) δ 13.09 (s, 1H), 9.50 (s, 1H), 8.69 (s, 1H), 8.41 (s, 1H), 8.30 (d, J=9.8 Hz, 1H), 8.12 (d, J=8.1 Hz, 1H), 8.00 (d, J=8.0 Hz, 1H), 7.52 (d, J=9.7 Hz, 1H), 5.09 (s, 1H), 3.3 (m, 1H), 1.76 (m, 4H), 1.28 (s, 6H), 1.12 (s, 6H), 1.01 (d, J=6.1 Hz, 2H), 0.65 (s, 2H).

### Example 64: Synthesis of Compounds 215, 226-228, 265, 266, 276, 277, and 285-289 Synthesis of Intermediate B164

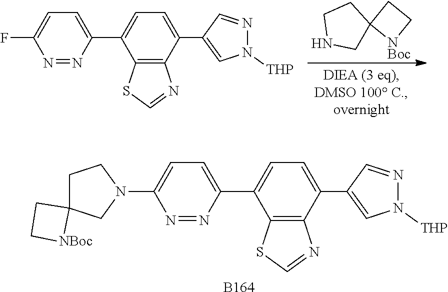

A mixture of 7-(6-fluoropyridazin-3-yl)-4-[1-(oxan-2-yl)pyrazol-4-yl]-1,3-benzothiazole (130 mg, 0.334 mmol, 1.00 equiv), tert-butyl 1,6-diazaspiro[3.4]octane-1-carboxylate (78.00 mg, 0.367 mmol, 1.1 equiv), and DIEA (129.50 mg, 1.002 mmol, 3 equiv) in DMSO (1.3 mL) was stirred overnight at 100° C. The reaction mixture was cooled to room temperature, then diluted with water (20 mL). A precipitate formed, and the solid was collected by filtration and washed with water (2×20 mL). The resulting mixture was concentrated under vacuum to afford tert-butyl 6-(6-{4-[1-(oxan-2-yl)pyrazol-4-yl]-1,3-benzothiazol-7-yl}pyridazin-3-yl)-1, 6-diazaspiro[3.4]octane-1-carboxylate (120 mg, 61.37%) as a solid. LCMS (ESI, m z): 574 [M+H]⁺.

### Synthesis of Compound 265

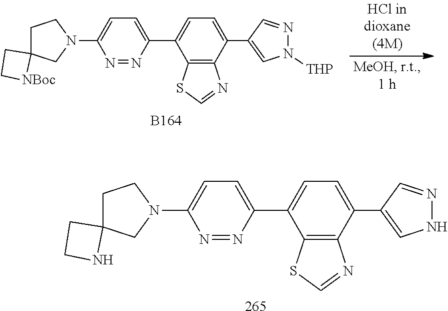

A mixture of tert-butyl 6-(6-{4-[1-(oxan-2-yl)pyrazol-4-yl]-1,3-benzothiazol-7-yl}pyridazin-3-yl)-1,6-diazaspiro[3.4]octane-1-carboxylate (30 mg, 0.051 mmol, 1.00 equiv) and HCl (gas) in 1,4-dioxane (0.25 mL) in methanol (0.90 mL) was stirred for 1 h at room temperature. The resulting mixture was concentrated under reduced pressure to give a residue. The residue was purified by prep-HPLC (Condition 4, Gradient 9) to afford 7-(6-{1,6-diazaspiro[3.4]octan-6-yl}pyridazin-3-yl)-4-(1H-pyrazol-4-yl)-1,3-benzothiazole (6.8 mg, 33.41%) as a solid. LCMS (ESI, m z): 390 [M+H]⁺.

### Synthesis of Compound 287

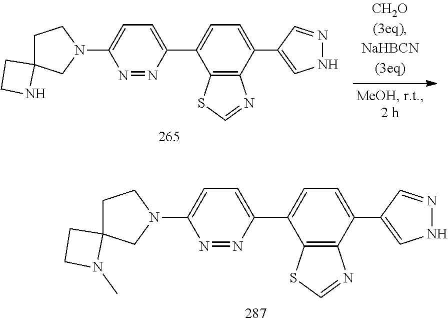 [SMILES: c1nc2c(-c3cn[nH]c3)ccc(-c3ccc(N4CCC5(CCN5)C4)nn3)c2s1 ; "265" ; CN1CCC12CCN(c1ccc(-c3ccc(-c4cn[nH]c4)c4ncsc34)nn1)C2 ; "287"]

To a stirred mixture of 7-(6-{1,6-diazaspiro[3.4]octan-6-yl}pyridazin-3-yl)-4-(1H-pyrazol-4-yl)-1,3-benzothiazole (60 mg, 0.151 mmol, 1.00 equiv) and formaldehyde (34.00 mg, 0.453 mmol, 3.00 equiv) in methanol (1.2 mL) was added NaBH₃CN (28.46 mg, 0.453 mmol, 3 equiv) in portions at room temperature. The resulting mixture was stirred for 2 h at room temperature, then concentrated under reduced pressure to give a residue. The residue was purified by prep-HPLC (Condition 7, Gradient 4) to afford 7-(6-{1-methyl-1,6-diazaspiro[3.4]octan-6-yl}pyridazin-3-yl)-4-(1H-pyrazol-4-yl)-1,3-benzothiazole (12.2 mg, 18.85%) as a solid. LCMS (ESI, m z): 404 [M+H]⁺.

Compounds 215, 226-228, 266, 276, 277, 285, 286, 288, and 289 were prepared according to the same procedure outlined in this Example XX and generalized by Scheme D. Table 4 below provides intermediates used in these procedures and final compound characterization data.

TABLE 4
Intermediates and characterization data for compounds prepared according to Example 64 protocol and general Scheme D

| | | LCMS | |
|---|---|---|---|
| | Coupling | (ESI, m/z) | ¹H NMR (400 MHz, |
| Compound Structure | Reagent | [M + H]⁺ | DMSO-d₆) δ |
| 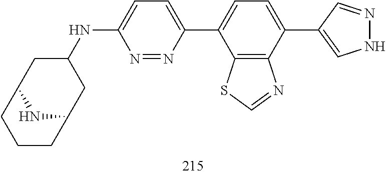 [SMILES: c1nc2c(-c3cn[nH]c3)ccc(-c3ccc(NC4C[C@H]5CCC[C@@H](C4)N5)nn3)c2s1] | 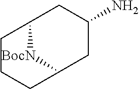 [SMILES: CC(C)(C)OC(=O)N1[C@@H]2CCC[C@H]1C[C@H](N)C2] | 418 | 9.42 (s, 1H), 8.46 (s, 2H), 8.03 (d, J = 9.5 Hz, 1H), 7.98 (d, J = 8.0 Hz, 1H), 7.92 (d, J = 7.9 Hz, 1H), 6.98 (d, J = 9.4 Hz, 1H), 4.90 (tt, J = 11.8, 5.9 Hz, 1H), 3.16 (m, 2H), 2.12 (dd, J = 13.0, 6.0 Hz, 2H), 1.99 (dp, J = 19.7, 6.7 Hz, 1H), 1.85 (tt, J = 11.6, 5.4 Hz, 2H), 1.68 (td, J = 12.2, 5.5 Hz, 5H) |
| 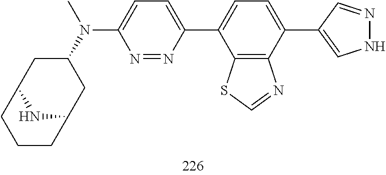 [SMILES: CN(c1ccc(-c2ccc(-c3cn[nH]c3)c3ncsc23)nn1)[C@@H]1C[C@H]2CCC[C@@H](C1)N2] | 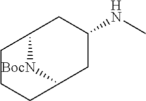 [SMILES: CN[C@@H]1C[C@H]2CCC[C@@H](C1)N2C(=O)OC(C)(C)C] | 432 | 9.97 (d, J = 10.8 Hz, 1H), 9.59 (s, 1H), 9.04 (d, J = 11.5 Hz, 1H), 8.60 (s, 3H), 8.29 (d, J = 8.1 Hz, 1H), 8.06 (d, J = 8.0 Hz, 1H), 7.87 (s, 1H), 5.40 (s, 1H), 3.75 (s, 2H), 3.21 (s, 3H), 2.56 (s, 2H), 2.10-1.97 (m, 5H), 1.92 (d, J = 12.3 Hz, 2H), 1.76-1.68 (m, 1H) |
| 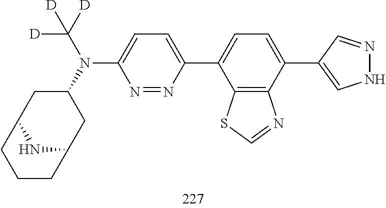 [SMILES: [2H]C([2H])([2H])N(c1ccc(-c2ccc(-c3cn[nH]c3)c3ncsc23)nn1)[C@@H]1C[C@H]2CCC[C@@H](C1)N2] | 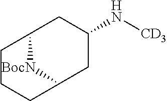 [SMILES: [2H]C([2H])([2H])N[C@@H]1C[C@H]2CCC[C@@H](C1)N2C(=O)OC(C)(C)C] | 435 | 9.73 (s, 1H), 9.57 (s, 1H), 8.94 (s, 1H), 8.59 (s, 2H), 8.54 (d, J = 10.0 Hz, 1H), 8.25 (d, J = 7.9 Hz, 1H), 8.05 (d, J = 7.9 Hz, 1H), 7.71 (s, 1H), 5.53 (s, 1H), 3.76 (s, 2H), 2.45 (dd, J = 13.0, 5.3 Hz, 2H), 2.07-1.90 (m, 7H), 1.75 (d, J = 12.6 Hz, 1H) |
| 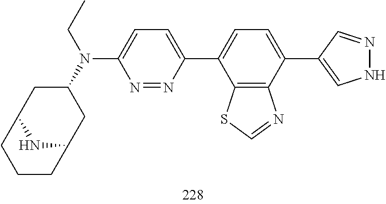 [SMILES: CCN(c1ccc(-c2ccc(-c3cn[nH]c3)c3ncsc23)nn1)[C@@H]1C[C@H]2CCC[C@@H](C1)N2] | 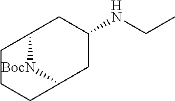 [SMILES: CCN[C@@H]1C[C@H]2CCC[C@@H](C1)N2C(=O)OC(C)(C)C] | 446 | 9.54 (m, 2H), 8.91 (s, 1H), 8.57 (s, 2H), 8.42 (d, J = 9.8 Hz, 1H), 8.19 (d, J = 8.1 Hz, 1H), 8.03 (d, J = 8.0 Hz, 1H), 7.49 (s, 1H), 5.68 (s, 1H), 3.77 (s, 2H), 3.67 (d, J = 7.3 Hz, 2H), 2.54 (s, 2H), 2.08-1.95 (m, 7H), 1.76 (d, J = 7.6 Hz, 1H), 1.20 (t, J = 6.9 Hz, 3H) |
| 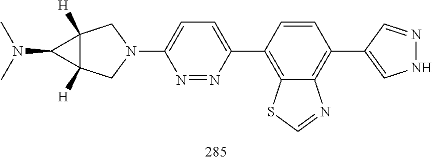 [SMILES: CN(C)[C@H]1[C@@H]2CN(c3ccc(-c4ccc(-c5cn[nH]c5)c5ncsc45)nn3)C[C@@H]21] | 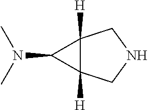 | 404 | 13.08 (s, 1H), 9.51 (s, 1H), 8.68 (s, 1H), 8.40 (s, 1H), 8.26 (d, J = 9.7 Hz, 1H), 8.11 (d, J = 8.1 Hz, 1H), 7.98 (d, J = 7.9 Hz, 1H), 7.05 (d, J = 9.7 Hz, 1H), 3.80 (d, J = 10.5 Hz, 2H), 3.57 (d, J = 10.6 Hz, 2H), 2.28 (s, 6H), 1.81 (s, 2H), 1.44 (t, J = 2.1 Hz, |
| | | | 1H) |
| 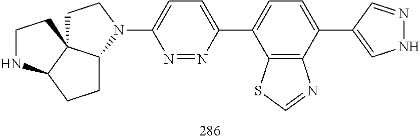 [SMILES: c1nc2c(-c3cn[nH]c3)ccc(-c3ccc(N4CC[C@@]56CCN[C@@H]5CC[C@@H]46)nn3)c2s1] | 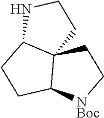 [SMILES: CC(C)(C)OC(=O)N1CC[C@]23CCN[C@H]2CC[C@H]13] | 430 | 12.87 (s, 1H), 9.45 (s, 1H), 8.49 (s, 2H), 8.16 (d, J = 9.6 Hz, 1H), 8.04 (d, J = 7.9 Hz, 1H), 7.95 (d, J = 8.0 Hz, 1H), 7.06 (d, J = 9.6 Hz, 1H), 4.11 (dd, J = 5.4, 2.9 Hz, 1H), 3.90 (ddd, J = 11.5, 7.9, 4.0 Hz, 1H), 3.56 (ddd, |
| | | | J = 10.8, 8.7, 6.8 Hz, 1H), |
| | | | 3.36 (dd, J = 6.4, 2.6 Hz, |
| | | | 1H), 2.96 (dt, J = 10.2, 6.3 |
| | | | Hz, 1H), 2.87 (dt, J = 10.2, |
| | | | 6.7 Hz, 1H), 2.25-2.11 (m, |
| | | | 2H), 2.10-1.91 (m, 2H), |
| | | | 1.90-1.78 (m, 2H), 1.74 |
| | | | (ddt, J = 13.2, 10.1, 6.8 Hz, |
| | | | 1H), 1.57 (ddt, J = 10.1, 7.1, |
| | | | 3.4 Hz, 1H) |
| 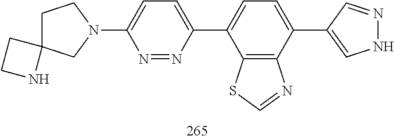 [SMILES: c1nc2c(-c3cn[nH]c3)ccc(-c3ccc(N4CCC5(CCN5)C4)nn3)c2s1] | 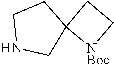 [SMILES: CC(C)(C)OC(=O)N1CCC12CCNC2] | 390 | 13.10 (s, 1H), 9.51 (s, 1H), 8.54 (s, 2H), 8.27 (d, J = 9.7 Hz, 1H), 8.11 (d, J = 8.1 Hz, 1H), 7.98 (d, J = 8.0 Hz, 1H), 7.05 (d, J = 9.6 Hz, 1H), 3.76-3.59 (m, 4H), 3.43-3.3 (q, J = 7.4 Hz, 2H), 2.42-2.32 (m, 1H), 2.26 |
| | | | (ddd, J = 10.9, 8.5, 5.5 Hz, |
| | | | 1H), 2.17 (t, J = 7.0 Hz, 2H) |
| 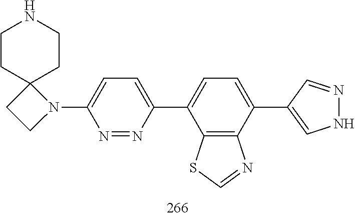 [SMILES: c1nc2c(-c3cn[nH]c3)ccc(-c3ccc(N4CCC45CCNCC5)nn3)c2s1] | 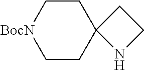 [SMILES: CC(C)(C)OC(=O)N1CCC2(CCN2)CC1] | 404 | 13.09 (s, 1H), 9.51 (s, 1H), 8.54 (s, 2H), 8.23 (d, J = 9.6 Hz, 1H), 8.10 (d, J = 8.0 Hz, 1H), 7.98 (d, J = 8.0 Hz, 1H), 6.91 (d, J = 9.5 Hz, 1H), 4.01 (t, J = 7.4 Hz, 2H), 2.95 (d, J = 12.1 Hz, 2H), 2.30 (m, 2H), 2.24-2.22 (m, 4H), 1.76 (d, J = 11.7 Hz, 2H) |
| 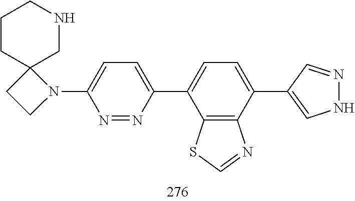 [SMILES: c1nc2c(-c3cn[nH]c3)ccc(-c3ccc(N4CCC45CCCNC5)nn3)c2s1] | 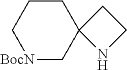 [SMILES: CC(C)(C)OC(=O)N1CCCC2(CCN2)C1] | 404 | 13.09 (s, 1H), 9.51 (s, 1H), 8.66 (s, 1H), 8.41 (s, 1H), 8.19 (d, J = 9.6 Hz, 1H), 8.09 (d, J = 8.1 Hz, 1H), 7.98 (d, J = 8.0 Hz, 1H), 6.95 (d, J = 9.6 Hz, 1H), 3.98 (t, J = 7.8 Hz, 2H), 3.23 (d, J = 12.1 Hz, 1H), 2.95 (dd, J = 11.7, 2.2 Hz, 1H), 2.81 (d, J = 12.0 Hz, 1H), 2.37-2.28 (m, 1H), 2.36-2.33 (m, 2H), 2.19 (dt, J = 10.9, 7.9 Hz, 1H), |
| | | | 1.90 (d, J = 12.4 Hz, 1H), |
| | | | 1.67-1.59 (m, 1H), 1.52- |
| | | | 1.38 (m, 1H) |
| 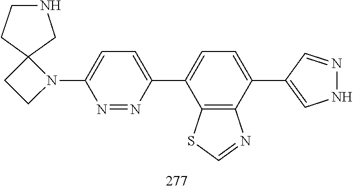 [SMILES: c1nc2c(-c3cn[nH]c3)ccc(-c3ccc(N4CCC45CCNC5)nn3)c2s1] | 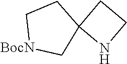 [SMILES: CC(C)(C)OC(=O)N1CCC2(CCN2)C1] | 390 | 12.87 (s, 1H), 9.45 (s, 1H), 8.49 (s, 2H), 8.15 (d, J = 9.5 Hz, 1H), 8.03 (d, J = 8.0 Hz, 1H), 7.95 (d, J = 7.9 Hz, 1H), 7.07 (d, J = 9.5 Hz, 1H), 4.10-3.95 (m, 2H), 3.43 (d, J = 11.5 Hz, 1H), 3.22-3.12 (m, 1H), 2.91- 2.80 (m, 2H), 2.48-2.23 (m, 3H), 1.97 (ddd, J = 12.6, 7.7, 4.1 Hz, 1H) |
| 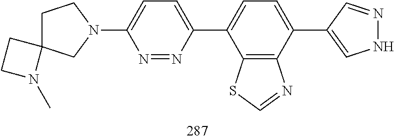 [SMILES: CN1CCC12CCN(c1ccc(-c3ccc(-c4cn[nH]c4)c4ncsc34)nn1)C2] | 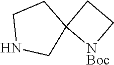 [SMILES: CC(C)(C)OC(=O)N1CCC12CCNC2] | 404 | 13.08 (s, 1H), 9.51 (s, 1H), 8.66 (s, 1H), 8.42 (s, 1H), 8.28 (d, J = 9.7 Hz, 1H), 8.12 (d, J = 8.0 Hz, 1H), 7.98 (d, J = 7.9 Hz, 1H), 7.08 (d, J = 9.6 Hz, 1H), 3.72 (s, 1H), 3.67 (d, J = 11.2 Hz, 1H), 3.56 (d, J = 10.9 Hz, 1H), |
| | | | 3.56-3.47 (m, 1H), 3.22- |
| | | | 3.12 (m, 1H), 3.12-3.02 (m, |
| | | | 1H), 2.27-2.12 (m, 1H), |
| | | | 2.18 (s, 3H), 2.15-2.13 (m, |
| | | | 1H), 2.11-2.07 (m, 2H) |
| 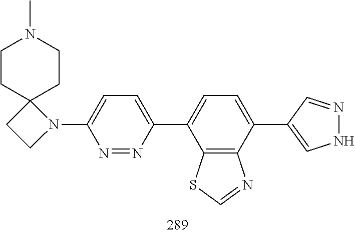 [SMILES: CN1CCC2(CC1)CCN2c1ccc(-c2ccc(-c3cn[nH]c3)c3ncsc23)nn1] | 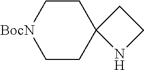 [SMILES: CC(C)(C)OC(=O)N1CCC2(CCN2)CC1] | 418 | 13.09 (s, 1H), 9.51 (s, 1H), 8.68 (s, 1H), 8.39 (d, J = 8.8 Hz, 1H), 8.23 (d, J = 9.6 Hz, 1H), 8.10 (d, J = 8.1 Hz, 1H), 7.98 (d, J = 7.9 Hz, 1H), 6.91 (d, J = 9.3 Hz, 1H), 4.00 (t, J = 7.4 Hz, 2H), 2.82 (s, 2H), 2.51-2.50 (m, 2H), 2.23-2.21 (m, 5H), 2.00 (s, 2H), 1.79 (d, J = 12.1 Hz, 2H) |
| 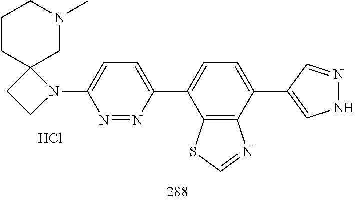 [SMILES: CN1CCCC2(CCN2c2ccc(-c3ccc(-c4cn[nH]c4)c4ncsc34)nn2)C1 ; Cl] | 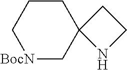 [SMILES: CC(C)(C)OC(=O)N1CCCC2(CCN2)C1] | 418 | 10.17 (s, 1H), 9.53 (s, 1H), 8.57 (s, 2H), 8.39 (d, J = 9.6 Hz, 1H), 8.18 (d, J = 8.1 Hz, 1H), 8.01 (d, J = 8.0 Hz, 1H), 7.16 (s, 1H), 4.08 (q, J = 7.1, 6.2 Hz, 2H), 3.78 (d, J = 10.3 Hz, 2H), 3.32 (d, J = 11.8 Hz, 1H), 3.14-3.04 (m, 1H), 2.84 (d, J = 4.5 Hz, 3H), 2.74 (d, J = 10.4 Hz, 1H), 2.51-2.50 (m, 1H), 2.33- 2.24 (m, 1H), 2.05-1.93 |
| | | | (m, 2H), 1.79 (q, J = 13.9 |
| | | | Hz, 1H) |

### Example 65: Synthesis of Compound 216

### Synthesis of Intermediate B165

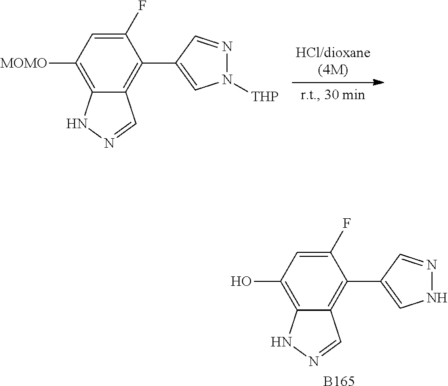

A solution of 5-fluoro-7-(methoxymethoxy)-4-[1-(oxan-2-yl)pyrazol-4-yl]-1H-indazole (800.00 mg, 2263.528 mmol, 1.00 equiv) in HCl (gas) in 1,4-dioxane (8 mL) was stirred for 30 min at room temperature. The resulting mixture was concentrated under reduced pressure to give a residue. The residue was purified by reverse flash chromatography (column, C18 silica gel; mobile phase A, water (10 mmol/L NH₄HCO₃), mobile phase B, acetonitrile; gradient, 5% B to 35% B in 20 min; detector, UV 220 nm) to afford 5-fluoro-4-(1H-pyrazol-4-yl)-1H-indazol-7-ol (350 mg, 69.45%) as a solid. LCMS (ES, m/z): 219 [M+H]⁺

### Synthesis of Intermediate B166

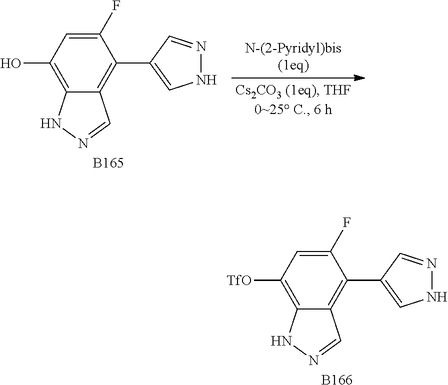 [SMILES: "B165" ; "B166" ; Oc1cc(F)c(-c2cn[nH]c2)c2cn[nH]c12 ; O=S(=O)(Oc1cc(F)c(-c2cn[nH]c2)c2cn[nH]c12)C(F)(F)F]

To a stirred mixture of 5-fluoro-4-(1H-pyrazol-4-yl)-1H-indazol-7-ol (350.00 mg, 1.572 mmol, 1.00 equiv) and Cs₂CO₃ (512 mg, 1.572 mmol, 1.00 equiv) in THE (12.00 mL) was added 1,1,1-trifluoro-N-(pyridin-2-yl)-N-trifluoromethanesulfonylmethanesulfonamide (563 mg, 1.572 mmol, 1.00 equiv) in THE (2 mL) dropwise at 0° C. The resulting mixture was stirred for 4 h at 0° C., then poured into water (50 mL) and extracted with ethyl acetate (2×50 mL). The combined organic layers were dried over anhydrous Na₂SO₄ and filtered. After filtration, the filtrate was concentrated under reduced pressure to give a residue. The residue was purified by silica gel column chromatography, eluted with PE/EA (1:1), to afford 5-fluoro-4-(1H-pyrazol-4-yl)-1H-indazol-7-yl trifluoromethanesulfonate (300 mg, 53.40%) as a solid. LCMS (ES, m/z): 351 [M+H]⁺.

### Synthesis of Intermediate B167

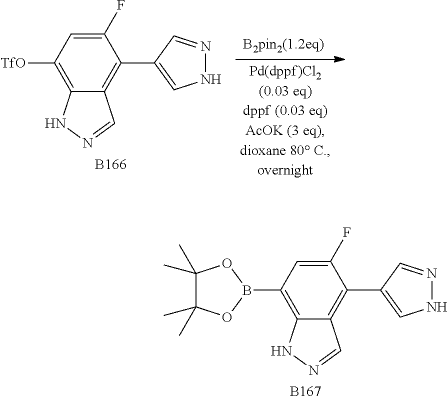

A mixture of 5-fluoro-4-(1H-pyrazol-4-yl)-1H-indazol-7-yl trifluoromethanesulfonate (300.00 mg, 0.839 mmol, 1.00 equiv), bis(pinacolato)diboron (319.74 mg, 1.259 mmol, 1.50 equiv), Pd(dppf)Cl₂ (68.38 mg, 0.084 mmol, 0.10 equiv), dppf(46.37 mg, 0.084 mmol, 0.10 equiv), and KOAc (247.14 mg, 2.518 mmol, 3.00 equiv) in 1,4-dioxane (15.00 mL) was stirred for 16 h at 80° C. under nitrogen atmosphere. The reaction mixture was cooled to room temperature, then poured into water (50 mL) and extracted with ethyl acetate (2×50 mL). The combined organic layers were dried over anhydrous Na₂SO₄, and filtered. After filtration, the filtrate was concentrated under reduced pressure to afford 5-fluoro-4-(1H-pyrazol-4-yl)-7-(4,4,5,5-tetramethyl-1,3,2-dioxaborolan-2-yl)-1H-indazole (200 mg, 71.16%) as a solid. LCMS (ES, m/z): 329 [M+H]⁺

### Synthesis of Intermediate B168

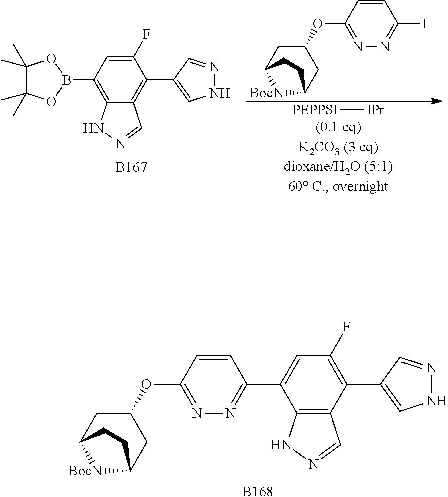

A mixture of 5-fluoro-4-(1H-pyrazol-4-yl)-7-(4,4,5,5-tetramethyl-1,3,2-dioxaborolan-2-yl)-1H-indazole (100.00 mg, 0.299 mmol, 1.00 equiv), tert-butyl (exo)-3-[(6-iodopyridazin-3-yl)oxy]-8-azabicyclo[3.2.1]octane-8-carboxylate (128.80 mg, 0.299 mmol, 1.00 equiv), [1,3-bis[2,6-bis(propan-2-yl)phenyl]-2,3-dihydro-1H-imidazol-2-yl]dichloro(3-chloropyridin-1-ium-1-yl)palladium (20.35 mg, 0.030 mmol, 0.10 equiv), and K₂CO₃ (82.55 mg, 0.598 mmol, 2.00 equiv) in 1,4-dioxane (5.00 mL) and H₂O (1.00 mL) was stirred for 16 h at 60° C. under nitrogen atmosphere. The resulting mixture was poured into water (50 mL) and extracted with ethyl acetate (2×50 mL). The combined organic layers were dried over anhydrous Na₂SO₄ and filtered. After filtration, the filtrate was concentrated under reduced pressure to give a residue. The residue was purified by silica gel column chromatography, eluted with PE/EA (1:1) to afford tert-butyl (exo)-3-([6-[5-fluoro-4-(1H-pyrazol-4-yl)-1H-indazol-7-yl]pyridazin-3-yl]oxy)-8-azabicyclo[3.2.1]octane-8-carboxylate (50 mg, 33.12%) as a solid. LCMS (ES, m/z): 506 [M+H]⁺.

### Synthesis of Compound 216

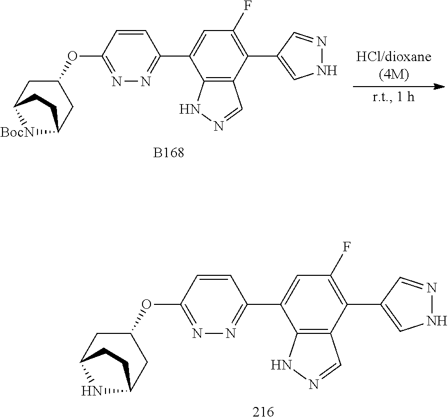

A solution of tert-butyl (exo)-3-([6-[5-fluoro-4-(1H-pyrazol-4-yl)-1H-indazol-7-yl]pyridazin-3-yl]oxy)-8-azabicyclo[3.2.1]octane-8-carboxylate (50.00 mg, 0.097 mmol, 1.00 equiv) in HCl (gas) in 1,4-dioxane (2.50 mL) was stirred for 1 h at room temperature, then concentrated under reduced pressure to give a residue. The residue was purified by prep-HPLC (Condition 7, Gradient 1) to afford 7-[6-[(exo)-8-azabicyclo[3.2.1]octan-3-yloxy]pyridazin-3-yl]-5-fluoro-4-(1H-pyrazol-4-yl)-1H-indazole (7.3 mg, 18.41%) as a solid. LCMS (ES, m/z): 406 [M+H]⁺. ¹H NMR (400 MHz, DMSO-d₆) δ 13.33 (s, 2H), 8.54 (s, 1H), 8.46 (d, J=9.4 Hz, 1H), 8.32 (s, 2H), 8.06 (d, J=12.4 Hz, 1H), 7.33 (d, J=9.4 Hz, 1H), 5.63 (tt, J=11.0, 5.8 Hz, 1H), 3.54 (d, J=4.4 Hz, 2H), 2.26-2.17 (m, 2H), 1.73 (dd, J=9.7, 6.6 Hz, 4H), 1.64 (td, J=11.8, 2.9 Hz, 2H).

### Example 66: Synthesis of Compound 290

### Synthesis of Intermediate B169

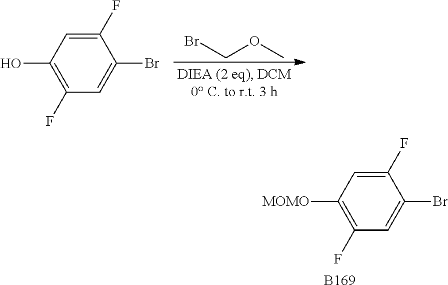

To a stirred solution of 4-bromo-2,5-difluorophenol (90.00 g, 430.643 mmol, 1.00 equiv) in DMF (500.00 mL) was added NaH (20.67 g, 861.285 mmol, 2.00 equiv) dropwise for 30 min at 0° C. To the reaction mixture was added bromomethoxymethane (80.72 g, 645.964 mmol, 1.50 equiv) in portions over 1 h at 25° C. The resulting mixture was diluted with water and extracted with ethyl acetate (3×1000 mL). The combined organic layers were washed with water (3×500 mL), dried over anhydrous Na₂SO₄, and filtered. After filtration, the filtrate was concentrated under reduced pressure to give a residue. The residue was purified by silica gel column chromatography, eluted with EA/PE (1:4) to afford 1-bromo-2,5-difluoro-4-(methoxymethoxy)benzene (98 g, 80.94%) as an oil.

### Synthesis of Intermediate B170

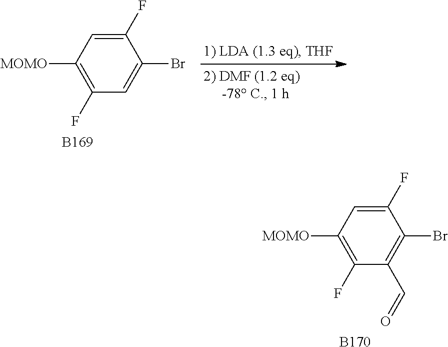

To a stirred solution of 1-bromo-2,5-difluoro-4-(methoxymethoxy)benzene (86 g, 339.863 mmol, 1.00 equiv) in THE (500 mL) was added LDA (40.05 g, 373.849 mmol, 1.1 equiv) in portions, and the reaction mixture was stirred for 30 min at −78° C. under N₂ atmosphere. To the reaction mixture was added DMF (27.33 g, 373.849 mmol, 1.1 equiv) in portions over the course of 1 h at −78° C. The reaction mixture was quenched with H₂O (500 mL) at 0° C. The resulting mixture was extracted with ethyl acetate (3×400 mL). The combined organic layers were washed with H₂O (3×300 mL), dried over anhydrous Na₂SO₄, and filtered. After filtration, the filtrate was concentrated under reduced pressure to give a residue. The residue was purified by silica gel column chromatography, eluted with EA/PE (1/10) to afford 2-bromo-3,6-difluoro-5-(methoxymethoxy)benzaldehyde (80 g, 83.75%) as a solid.

### Synthesis of Intermediate B171

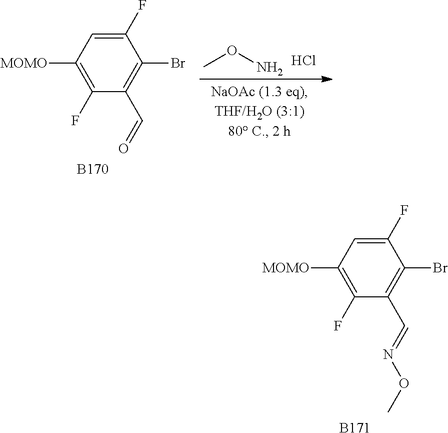

A solution of 2-bromo-3,6-difluoro-5-(methoxymethoxy)benzaldehyde (80 g, 284.644 mmol, 1.00 equiv) in THF (300 mL) and H₂O (100 mL) was treated with NaOAc (30.36 g, 370.037 mmol, 1.3 equiv), and the reaction mixture was stirred for 30 min at 25° C. under nitrogen atmosphere. To the reaction mixture was added O-methylhydroxylamine (16.07 g, 341.573 mmol, 1.2 equiv) dropwise, and the reaction mixture was stirred for 2 h at 80° C. The mixture was cooled to 25° C. The resulting mixture was extracted with ethyl acetate (3×300 mL). The combined organic layers were washed with H₂O (3×300 mL), dried over anhydrous Na₂SO₄, and filtered. After filtration, the filtrate was concentrated under reduced pressure to give a residue. The residue was purified by silica gel column chromatography, eluted with EA/PE (1:3) to afford (E)-2-bromo-3,6-difluoro-5-(methoxymethoxy)benzaldehyde O-methyl oxime (90 g, 101.96%) as a solid. LCMS (ES, m/z): 295 [M+H]⁺.

### Synthesis of Intermediate B172

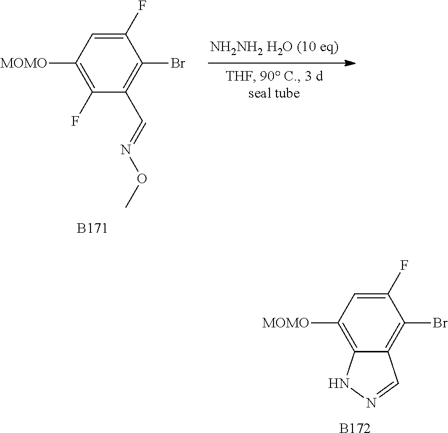

A mixture of (E)-2-bromo-3,6-difluoro-5-(methoxymethoxy)benzaldehyde O-methyl oxime (88 g, 283.784 mmol, 1.00 equiv) and NH₂NH₂.H₂O (142.06 g, 2837.840 mmol, 10 equiv) in THF (800 mL) was stirred for 3 days at 90° C. The reaction mixture was cooled to 25° C. The resulting mixture was extracted with ethyl acetate (3×800 mL). The combined organic layers were washed with H₂O (3×500 mL), dried over anhydrous Na₂SO₄, and filtered. After filtration, the filtrate was concentrated under reduced pressure to give a residue. The residue was purified by silica gel column chromatography, eluted with EA/PE (1:3) to afford 4-bromo-5-fluoro-7-(methoxymethoxy)-1H-indazole (36 g, 46.12%) as a solid. LCMS (ES, m/z): 275 [M+H]⁺.

### Synthesis of Intermediate B173

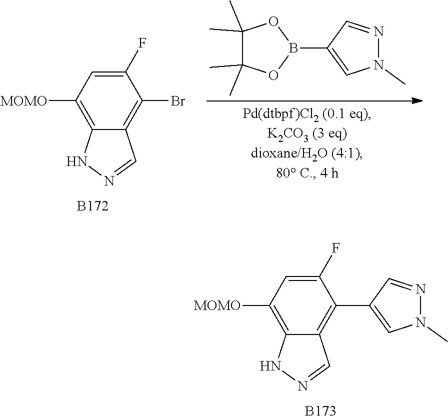

To a stirred mixture of 4-bromo-5-fluoro-7-(methoxymethoxy)-1H-indazole (4 g, 14.541 mmol, 1.00 equiv) and 1-methyl-4-(4,4,5,5-tetramethyl-1,3,2-dioxaborolan-2-yl)pyrazole (4.54 g, 21.812 mmol, 1.5 equiv) in dioxane (10 mL) and H₂O (2 mL) was added Pd(dtbpf)Cl₂ (0.95 g, 1.454 mmol, 0.1 equiv) and K₂CO₃ (6.03 g, 43.623 mmol, 3 equiv). The reaction mixture was stirred for 4 h at 80° C. under N₂ atmosphere, then cooled to 25° C. The resulting mixture was extracted with ethyl acetate (3×15 mL). The combined organic layers were washed with H₂O (3×10 mL), dried over anhydrous Na₂SO₄, and filtered. After filtration, the filtrate was concentrated under reduced pressure to give a residue. The residue was purified by silica gel column chromatography, eluted with EA/PE (1:3) to afford 5-fluoro-7-(methoxymethoxy)-4-(1-methylpyrazol-4-yl)-1H-indazole (3.1 g, 77.17%) as a solid. LCMS (ES, m/z): 277 [M+H]⁺.

### Synthesis of Intermediate B174

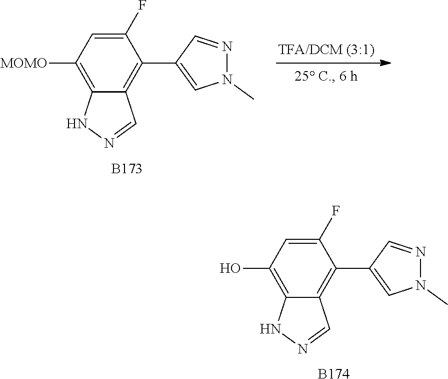

A mixture of 5-fluoro-7-(methoxymethoxy)-4-(1-methylpyrazol-4-yl)-1H-indazole (3.1 g, 11.221 mmol, 1.00 equiv) and TFA (10 mL, 134.630 mmol, 12.00 equiv) in DCM (30 mL) was stirred for 6 h at 25° C. The resulting mixture was concentrated under reduced pressure to give a residue. The residue was purified by reverse flash chromatography (column, silica gel; mobile phase, acetonitrile in water (10 mmol/L NH₄HCO₃); gradient, 10% to 50% in 10 min; detector, UV 254 nm) to afford 5-fluoro-4-(1-methylpyrazol-4-yl)-1H-indazol-7-ol (1.6 g, 61.40%) as a solid. LCMS (ES, m/z): 233 [M+H]⁺.

### Synthesis of Intermediate B175

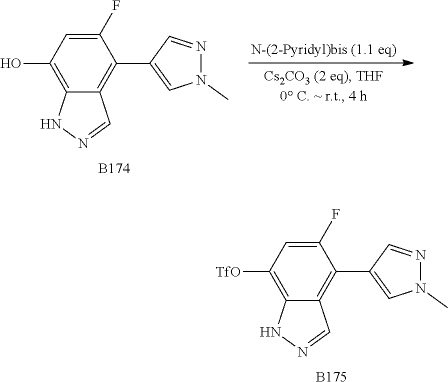 [SMILES: "B174" ; "B175" ; Cn1cc(-c2c(F)cc(O)c3[nH]ncc23)cn1 ; Cn1cc(-c2c(F)cc(OS(=O)(=O)C(F)(F)F)c3[nH]ncc23)cn1]

A solution of 5-fluoro-4-(1-methylpyrazol-4-yl)-1H-indazol-7-ol (2.8 g, 12.058 mmol, 1.00 equiv) in THE (6 mL) was treated with Cs₂CO₃ (7.86 g, 24.116 mmol, 2 equiv). The reaction mixture was stirred for 30 min at 0° C. under nitrogen atmosphere. To the reaction mixture was added 1,1,1-trifluoro-N-phenyl-N-trifluoromethanesulfonylmethanesulfonamide (4.74 g, 13.264 mmol, 1.1 equiv) dropwise, and the reaction mixture was stirred for an additional 4 h at 25° C. The resulting mixture was extracted with ethyl acetate (3×20 mL). The combined organic layers were washed with H₂O (3×20 mL), dried over anhydrous Na₂SO₄, and filtered. After filtration, the filtrate was concentrated under reduced pressure to give a residue. The residue was purified by reverse flash chromatography (column, silica gel; mobile phase, MeCN in water (10 mmol/L NH₄HCO₃); gradient, 10% to 50% in 10 min; detector, UV 254 nm) to afford 5-fluoro-4-(1-methylpyrazol-4-yl)-1H-indazol-7-yl trifluoromethanesulfonate (1.1 g, 25.04%) as a solid. LCMS (ES, m/z): 365 [M+H]⁺.

### Synthesis of Intermediate B176

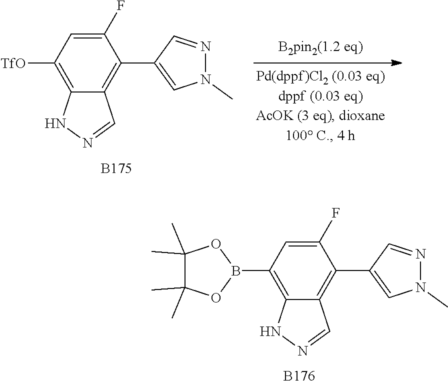

To a stirred mixture of 5-fluoro-4-(1-methylpyrazol-4-yl)-1H-indazol-7-yl trifluoromethanesulfonate (1 g, 2.745 mmol, 1.00 equiv) and bis(pinacolato)diboron (1.05 g, 4.117 mmol, 1.5 equiv) in dioxane (10 mL, 118.041 mmol, 43.00 equiv) was added Pd(dppf)Cl₂ (0.20 g, 0.275 mmol, 0.1 equiv), dppf (0.30 g, 0.549 mmol, 0.2 equiv), and AcOK (0.54 g, 5.490 mmol, 2 equiv). The reaction mixture was stirred for 4 h at 100° C. under N₂ atmosphere, then cooled to 25° C. and extracted with ethyl acetate (3×20 mL). The combined organic layers were washed with H₂O (3×15 mL), dried over anhydrous Na₂SO₄, and filtered. After filtration, the filtrate was concentrated under reduced pressure to give a residue. The residue was purified by silica gel column chromatography, eluted with EA/PE (4:1) to afford 5-fluoro-4-(1-methylpyrazol-4-yl)-7-(4,4,5,5-tetramethyl-1,3,2-dioxaborolan-2-yl)-1H-indazole (700 mg, 74.52%) as a solid. LCMS (ES, m/z): 343 [M+H]⁺.

### Synthesis of Intermediate B177

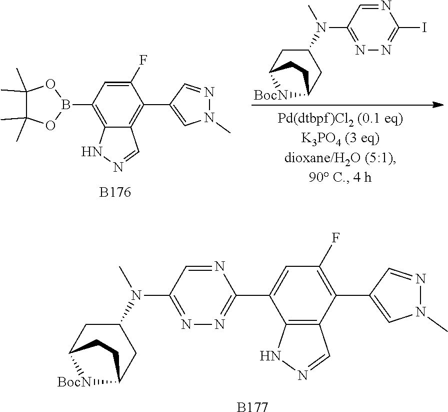

To a stirred mixture of 5-fluoro-4-(1-methylpyrazol-4-yl)-7-(4,4,5,5-tetramethyl-1,3,2-dioxaborolan-2-yl)-1H-indazole (100 mg, 0.292 mmol, 1.00 equiv) and tert-butyl (exo)-3-[(3-iodo-1,2,4-triazin-6-yl)(methyl)amino]-8-azabicyclo[3.2.1]octane-8-carboxylate (156.17 mg, 0.350 mmol, 1.2 equiv) in dioxane (5 mL) and H₂O (1 mL) was added Pd(dtbpf)Cl₂ (19.05 mg, 0.029 mmol, 0.1 equiv) and K₃PO₄ (124.07 mg, 0.584 mmol, 2 equiv). The reaction mixture was stirred for 4 h at 90° C. under N₂ atmosphere, then cooled to 25° C. The resulting mixture was extracted with ethyl acetate (3×20 mL). The combined organic layers were washed with H₂O (3×10 mL), dried over anhydrous Na₂SO₄, and filtered. After filtration, the filtrate was concentrated under reduced pressure to give a residue. The residue was purified by silica gel column chromatography, eluted with EA/PE (1:1) to afford tert-butyl (exo)-3-({3-[5-fluoro-4-(1-methylpyrazol-4-yl)-1H-indazol-7-yl]-1,2,4-triazin-6-yl}(methyl)amino)-8-azabicyclo[3.2.1]octane-8-carboxylate (66 mg, 42.32%) as a solid. LCMS (ES, m/z): 534 [M+H]⁺.

### Synthesis of Compound 290

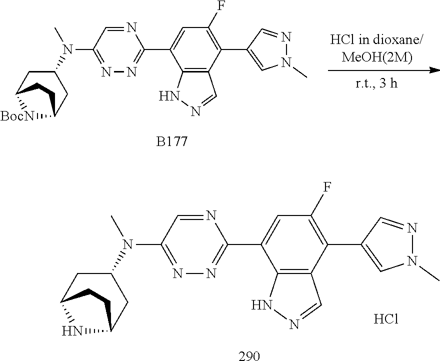

A mixture of tert-butyl (exo)-3-({3-[5-fluoro-4-(1-methylpyrazol-4-yl)-1H-indazol-7-yl]-1,2,4-triazin-6-yl}(methyl)amino)-8-azabicyclo[3.2.1]octane-8-carboxylate (60 mg, 0.112 mmol, 1.00 equiv) and HCl (2 mL, 65.824 mmol, 585.41 equiv) in dioxane (6 mL) was stirred at room temperature. The resulting mixture was concentrated under reduced pressure to give a residue. The residue was purified by prep-HPLC (Condition 8, Gradient 3) to afford (exo)-N-{3-[5-fluoro-4-(1-methylpyrazol-4-yl)-1H-indazol-7-yl]-1,2,4-triazin-6-yl}-N-methyl-8-azabicyclo[3.2.1]octan-3-amine hydrochloride (22.5 mg, 42.58%) as a solid. LCMS (ES, m/z): 434 [M+H]⁺. 1H NMR (400 MHz, DMSO-d₆) δ 13.23 (s, 1H), 9.43 (d, J=10.3 Hz, 1H), 9.24 (s, 1H), 9.01 (s, 1H), 8.53 (s, 1H), 8.45 (d, J=1.5 Hz, 1H), 8.10 (d, J=2.1 Hz, 1H), 8.03 (d, J=12.4 Hz, 1H), 5.26 (m, 1H), 4.14 (s, 2H), 3.99 (s, 3H), 3.19 (s, 3H), 2.41-2.29 (m, 2H), 2.10 (dd, J=9.2, 4.5 Hz, 2H), 1.97 (t, J=6.9 Hz, 2H), 1.81 (d, J=12.7 Hz, 2H). ¹⁹F NMR (376 MHz, DMSO-d₆) δ−124.87.

### Example 67: Synthesis of Compound 291

### Synthesis of Intermediate B178

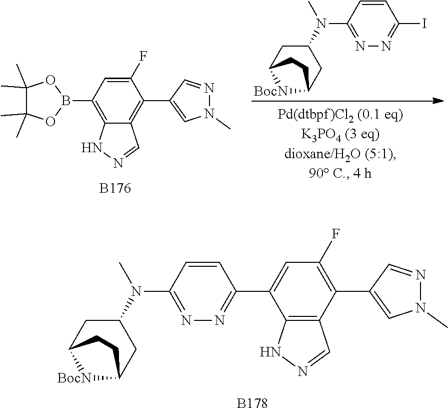

To a stirred mixture of 5-fluoro-4-(1-methylpyrazol-4-yl)-7-(4,4,5,5-tetramethyl-1,3,2-dioxaborolan-2-yl)-1H-indazole (100 mg, 0.292 mmol, 1.00 equiv) and tert-butyl (exo)-3-[(6-iodopyridazin-3-yl)(methyl)amino]-8-azabicyclo[3.2.1]octane-8-carboxylate (155.82 mg, 0.350 mmol, 1.2 equiv) in dioxane (5 mL) and H₂O (1 mL) was added Pd(dtbpf)Cl₂ (19.05 mg, 0.029 mmol, 0.1 equiv) and K₃PO₄ (124.07 mg, 0.584 mmol, 2 equiv). The reaction mixture was stirred for 4 h at 90° C. under N₂ atmosphere, then cooled to 25° C. The resulting mixture was extracted with ethyl acetate (3×20 mL). The combined organic layers were washed with H₂O (3×10 mL), dried over anhydrous Na₂SO₄, and filtered. After filtration, the filtrate was concentrated under reduced pressure to give a residue. The residue was purified by silica gel column chromatography, eluted with EA/PE (1:1) to afford tert-butyl (exo)-3-({6-[5-fluoro-4-(1-methylpyrazol-4-yl)-1H-indazol-7-yl]pyridazin-3-yl}(methyl)amino)-8-azabicyclo[3.2.1]octane-8-carboxylate (70 mg, 44.97%) as a solid. LCMS (ES, m/z): 533 [M+H]⁺.

### Synthesis of Compound 291

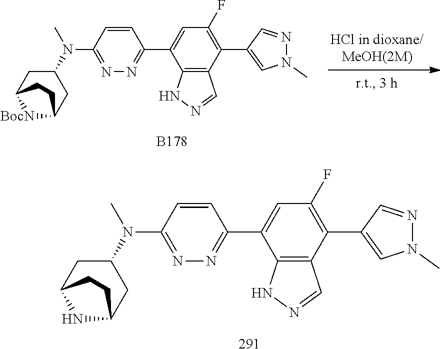

A solution of tert-butyl (exo)-3-({6-[5-fluoro-4-(1-methylpyrazol-4-yl)-1H-indazol-7-yl]pyridazin-3-yl}(methyl)amino)-8-azabicyclo[3.2.1]octane-8-carboxylate (75 mg, 0.141 mmol, 1.00 equiv) and HCl (2 mL, 65.824 mmol, 467.46 equiv) in dioxane (4.00 mL) was stirred for 3h at 25° C. The resulting mixture was concentrated. The crude product was purified by prep-HPLC (Condition 4, Gradient 10) to afford (exo)-N-{6-[5-fluoro-4-(1-methylpyrazol-4-yl)-1H-indazol-7-yl]pyridazin-3-yl}-N-methyl-8-azabicyclo[3.2.1]octan-3-amine (23.1 mg, 37.93%) as a solid. LCMS (ES, m/z): 433 [M+H]⁺. ¹H NMR (400 MHz, DMSO-d₆) δ 13.21 (s, 1H), 8.49 (s, 1H), 8.43 (s, 1H), 8.21 (d, J=9.8 Hz, 1H), 8.08 (d, J=2.1 Hz, 1H), 7.94 (d, J=12.6 Hz, 1H), 7.26 (d, J=9.7 Hz, 1H), 5.08 (s, 1H), 3.98 (s, 3H), 3.55 (s, 2H), 2.98 (s, 3H), 1.89-1.80 (m, 2H), 1.78 (s, 4H), 1.58 (s, 2H). ¹⁹F NMR (376 MHz, DMSO-d₆): δ−125.05.

### Example 68: Synthesis of Compounds

### Synthesis of Intermediate B179

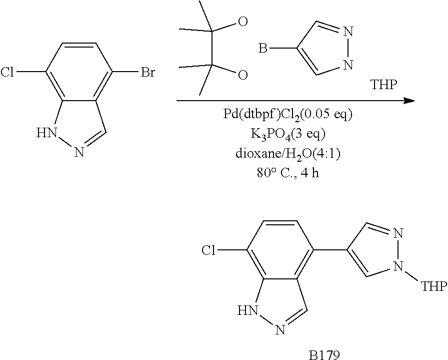

A mixture of 4-bromo-7-chloro-1H-indazole (2 g, 8.467 mmol, 1.00 equiv) and 1-(oxan-2-yl)-4-(4,4,5,5-tetramethyl-1,3,2-dioxaborolan-2-yl)pyrazole (2.40 g, 8.467 mmol, 1.00 equiv), Pd(dtbpf)Cl₂ (0.84 g, 1.270 mmol, 0.15 equiv), and K₃PO₄ (5.50 g, 25.401 mmol, 3.00 equiv) in 1,4-dioxanedioxane (80 mL) and H₂O (20 mL) was stirred for 16 h at 80° C. under nitrogen atmosphere. The resulting mixture was extracted with ethyl acetate (2×200 mL). The combined organic layers were dried over anhydrous Na₂SO₄ and filtered. After filtration, the filtrate was concentrated under reduced pressure to give a residue. The residue was purified by silica gel column chromatography, eluted with PE/EA (1:01) to afford 7-chloro-4-[1-(oxan-2-yl)pyrazol-4-yl]-1H-indazole (7.3 mg, 0.06%) as a solid. LCMS (ES, m/z): 303 [M+H]⁺.

### Synthesis of Intermediate B180

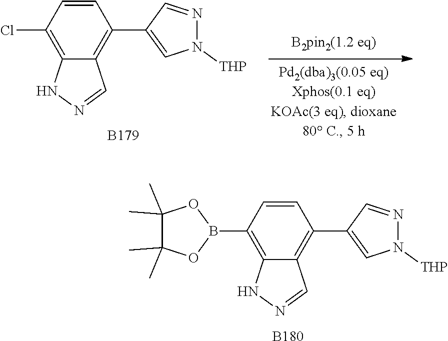

A mixture of 7-chloro-4-[1-(oxan-2-yl)pyrazol-4-yl]-1H-indazole (7.3 g, 24.112 mmol, 1.00 equiv), 4,4,5,5-tetramethyl-2-(tetramethyl-1,3,2-dioxaborolan-2-yl)-1,3,2-dioxaborolane (12.25 g, 48.224 mmol, 2 equiv), Pd₂(dba)₃ (1.10 g, 1.206 mmol, 0.05 equiv), KOAc (7.10 g, 72.336 mmol, 3 equiv), and XPhos (11.49 g, 24.112 mmol, 1equiv) in dioxane (73 mL) was stirred for 5 h at 80° C. under nitrogen atmosphere. The reaction mixture was cooled to room temperature, diluted with water, and extracted with ethyl acetate (2×200 mL). The combined organic layers were dried over anhydrous Na₂SO₄ and filtered. After filtration, the filtrate was concentrated under reduced pressure to give a residue. The residue was purified by silica gel column chromatography, eluted with PE/EA (1:1) to afford 4-[1-(oxan-2-yl)pyrazol-4-yl]-7-(4,4,5,5-tetramethyl-1,3,2-dioxaborolan-2-yl)-1H-indazole as an oil. LCMS (ES, m/z): 395 [M+H]⁺.

### Synthesis of Intermediate B181

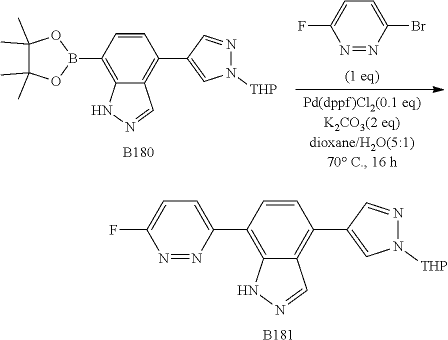

A mixture of 3-bromo-6-fluoropyridazine (1.5 g, 8.476 mmol, 1.00 equiv) and 4-[1-(oxan-2-yl)pyrazol-4-yl]-7-(4,4,5,5-tetramethyl-1,3,2-dioxaborolan-2-yl)-1H-indazole (3.34 g, 8.476 mmol, 1.00 equiv), K₂CO₃ (2.34 g, 16.952 mmol, 2 equiv), and Pd(dppf)Cl₂ (0.62 g, 0.848 mmol, 0.1 equiv) in dioxane (15 mL) and H₂O (3 mL) was stirred for 16 h at 70° C. under nitrogen atmosphere. The reaction mixture was cooled to room temperature and extracted with ethyl acetate (1×50 mL). The combined organic layers were dried over anhydrous Na₂SO₄ and filtered. After filtration, the filtrate was concentrated under reduced pressure to give a residue. The residue was re-crystallized from PE/EA (5:01 18 mL) to afford 7-(6-fluoropyridazin-3-yl)-4-[1-(oxan-2-yl)pyrazol-4-yl]-1H-indazole (3 g, 97.14%) as a solid. LCMS (ES, m/z): 365 [M+H]⁺.

### Synthesis of Intermediate B182

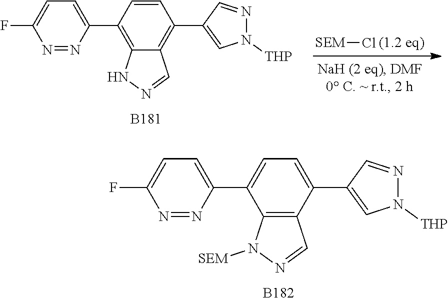

A solution of 7-(6-fluoropyridazin-3-yl)-4-[1-(oxan-2-yl)pyrazol-4-yl]-1H-indazole (2.5 g, 6.861 mmol, 1.00 equiv) in DMF (25 mL, 323.044 mmol, 47.08 equiv) was treated with NaH (0.33 g, 13.722 mmol, 2 equiv). The reaction mixture was stirred for 30 min at 0° C. under nitrogen atmosphere. To the reaction mixture was added SEM-Cl (1.37 g, 8.233 mmol, 1.2 equiv) dropwise at room temperature. The resulting mixture was poured into water (100 mL) and extracted with ethyl acetate (2×100 mL). The combined organic layers were washed with brine (3×100 mL), dried over anhydrous Na₂SO₄, and filtered. After filtration, the filtrate was concentrated under reduced pressure to give a residue. The residue was re-crystallized from PE/EA (5:1, 30 mL) to afford 7-(6-fluoropyridazin-3-yl)-4-[1-(oxan-2-yl)pyrazol-4-yl]-1-{[2-(trimethylsilyl)ethoxy]methyl}indazole (3 g, 88.40%) as a solid. LCMS (ES, m/z): 495 [M+H]⁺.

### Synthesis of Intermediate B183

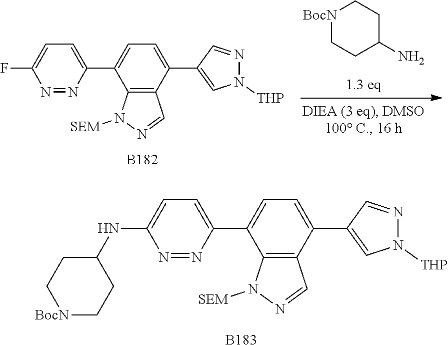

A mixture of 7-(6-fluoropyridazin-3-yl)-4-[1-(oxan-2-yl)pyrazol-4-yl]-1-{[2-(trimethylsilyl)ethoxy]methyl}indazole (500 mg, 1.011 mmol, 1.00 equiv), tert-butyl 4-aminopiperidine-1-carboxylate (263.18 mg, 1.314 mmol, 1.3 equiv), and DIEA in DMSO (5 mL, 70.393 mmol, 69.64 equiv) was stirred for 16 h at 100° C. The reaction mixture was cooled to room temperature, diluted with water, and extracted with ethyl acetate (2×20 mL). The combined organic layers were washed with brine (3×20 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[(6-{4-[1-(oxan-2-yl)pyrazol-4-yl]-1-{[2-(trimethylsilyl)ethoxy]methyl}indazol-7-yl}pyridazin-3-yl)amino]piperidine-1-carboxylate (262 mg, 38.40%) as a solid. LCMS (ES, m/z): 675 [M+H]⁺.

### Synthesis of Compound 263

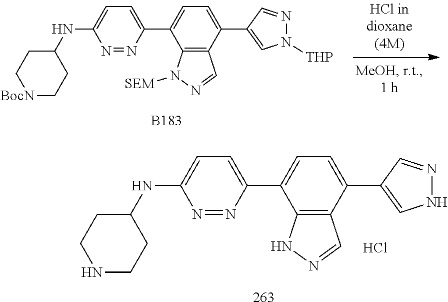

A solution of tert-butyl 4-[(6-{4-[1-(oxan-2-yl)pyrazol-4-yl]-1-{[2-(trimethylsilyl)ethoxy]methyl}indazol-7-yl}pyridazin-3-yl)amino]piperidine-1-carboxylate (80 mg, 0.119 mmol, 1.00 equiv), methanol (1.14 mL, 28.339 mmol, 238.14 equiv), and HCl (gas) in 1,4-dioxane (1.14 mL, 37.762 mmol, 317.33 equiv) was stirred for 1 h at room temperature. The resulting mixture was concentrated under reduced pressure to give a residue. The residue purified by prep-HPLC (Condition 8, Gradient 1, Gradient 2) to afford N-(piperidin-4-yl)-6-[4-(1H-pyrazol-4-yl)-1H-indazol-7-yl]pyridazin-3-amine hydrogen chloride (4.3 mg, 9.92%) as a solid. LCMS (ES, m/z): 361 [M+H]⁺.

Compounds 215, 226-228, 266, 276, 277, 285, 286, 288, and 289 were prepared according to the same procedure outlined in this Example XX and generalized by Scheme E. Table 6 below provides intermediates used in these procedures and final compound characterization data.

TABLE 6
Intermediates and characterization data for compounds prepared according to Example
68 protocol and general Scheme E.

| | LCMS (ESI | ¹H NMR (400 MHz, |
|---|---|---|
| Compound Structure | m/z) [M + H]⁺ | DMSO-d₆, D₂O) δ |
| 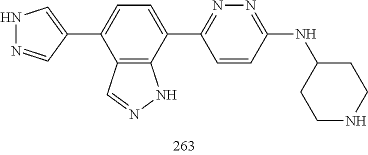 [SMILES: c1n[nH]cc1-c1ccc(-c2ccc(NC3CCNCC3)nn2)c2[nH]ncc12] | 361 | 8.63 (d, J = 3.0 Hz, 1H), 8.56 (d, J = 9.5 Hz, 1H), 8.40 (d, J = 2.6 Hz, 2H), 8.03 (d, J = 7.7 Hz, 1H), 7.66 (s, 1H), 7.57 (d, J = 7.6 Hz, 1H), 4.14 (s, 1H), 3.4 (m, 2H), 3.05 (td, J = 12.6, 3.1 Hz, 2H), 2.20 (dd, J = 14.2, 3.8 Hz, 2H), 1.83 (s, |
| | | 2H) |
| 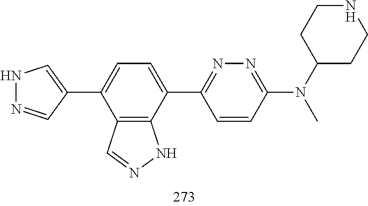 [SMILES: CN(c1ccc(-c2ccc(-c3cn[nH]c3)c3cn[nH]c23)nn1)C1CCNCC1] | 375 | 13.00 (s, 1H), 8.9-8.7 (m, 2H), 8.76 (s, 1H), 8.59 (s, 1H), 8.38 (s, 2H), 8.02 (d, J = 7.7 Hz, 1H), 7.72 (s, 1H), 7.53 (d, J = 7.6 Hz, 1H), 4.80 (s, 1H), 3.44 (d, J = 12.4 Hz, 2H), 3.09 (s, 5H), 2.12 (tt, J = 13.0, 7.4 Hz, 2H), 1.90 (d, J = 12.5 Hz, 2H) |
| 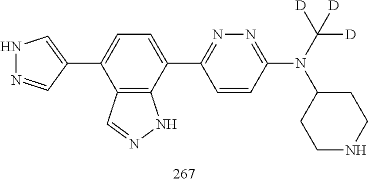 [SMILES: [2H]C([2H])([2H])N(c1ccc(-c2ccc(-c3cn[nH]c3)c3cn[nH]c23)nn1)C1CCNCC1] | 378 | 13.19 (s, 1H), 13.07 (s, 1H), 8.50 (s, 1H), 8.33 (s, 2H), 8.17 (d, J = 9.8 Hz, 1H), 7.91 (d, J = 7.6 Hz, 1H), 7.47 (d, J = 7.6 Hz, 1H), 7.28 (d, J = 9.8 Hz, 1H), 4.71-4.60 (m, 1H), 3.05 (d, J = 12.4 Hz, 2H), 2.62 (d, J = 2.7 Hz, 2H), 1.77-1.58 (m, 4H) |
| 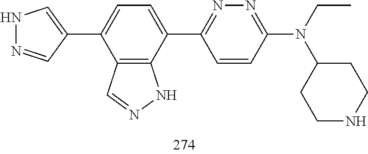 [SMILES: CCN(c1ccc(-c2ccc(-c3cn[nH]c3)c3cn[nH]c23)nn1)C1CCNCC1] | 389 | 13.18 (s, 1H), 13.06 (s, 1H), 8.50 (s, 1H), 8.32 (s, 2H), 8.14 (d, J = 9.7 Hz, 1H), 7.89 (d, J = 7.7 Hz, 1H), 7.47 (d, J = 7.6 Hz, 1H), 7.22 (d, J = 9.8 Hz, 1H), 4.66 (s, 1H), 3.56 (q, J = 7.0 Hz, 2H), 3.05 (dt, J = 12.3, 3.4 Hz, 2H), 2.63 (d, J = |
| | | 13.8 Hz, 2H), 1.68 (h, J = |
| | | 3.9 Hz, 4H), 1.20 (t, J = 6.9 |
| | | Hz, 3H) |
| 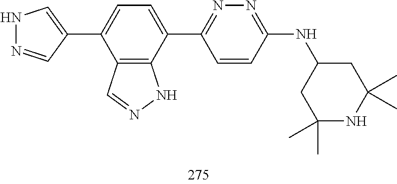 [SMILES: CC1(C)CC(Nc2ccc(-c3ccc(-c4cn[nH]c4)c4cn[nH]c34)nn2)CC(C)(C)N1] | 417 | 13.19 (s, 1H), 13.02 (s, 1H), 8.50 (d, J = 1.5 Hz, 1H), 8.19 (s, 1H), 8.05 (d, J = 9.5 Hz, 1H), 7.84 (d, J = 7.7 Hz, 1H), 7.46 (d, J = 7.6 Hz, 1H), 6.95 (d, J = 9.5 Hz, 1H), 6.85 (d, J = 7.9 Hz, 1H), 4.47 (s, 1H), 1.93 (dd, J = 12.2, 3.7 Hz, 2H), 1.25 (s, 6H), 1.07 (s, 6H), 1.04 (dd, J = 12.2, 3.7 Hz, 2H) |
| 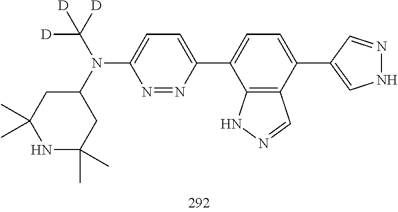 [SMILES: [2H]C([2H])([2H])N(c1ccc(-c2ccc(-c3cn[nH]c3)c3cn[nH]c23)nn1)C1CC(C)(C)NC(C)(C)C1] | 434 | 13.19 (s, 1H), 13.07 (s, 1H), 8.51 (d, J = 1.6 Hz, 2H), 8.17 (d, J = 9.8 Hz, 2H), 7.89 (d, J = 7.7 Hz, 1H), 7.47 (d, J = 7.6 Hz, 1H), 7.25 (d, J = 9.8 Hz, 1H), 5.06 (s, 1H), 1.56 (dd, J = 12.0, 3.6 Hz, 2H), 1.46 (t, J = 12.1 Hz, 2H), 1.29 (s, 6H), 1.25 (d, J = 6.2 Hz, 1H), 1.11 (s, 6H) |
| 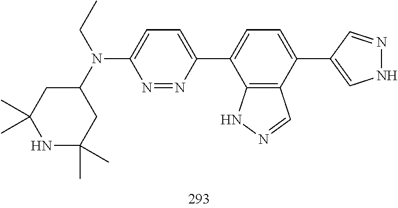 [SMILES: CCN(c1ccc(-c2ccc(-c3cn[nH]c3)c3cn[nH]c23)nn1)C1CC(C)(C)NC(C)(C)C1] | 445 | 13.11 (s, 1H), 9.10 (d, J = 12.1 Hz, 1H), 8.57 (s, 1H), 8.36 (s, 2H), 8.31 (d, J = 9.8 Hz, 1H), 8.23 (d, J = 12.1 Hz, 1H), 7.96 (d, J = 7.7 Hz, 1H), 7.51 (d, J = 7.7 Hz, 2H), 5.16 (s, 1H), 3.59 (q, J = 6.9 Hz, 2H), 2.05 (t, J = 12.8 Hz, 2H), 1.88 (dd, J = 13.4, 3.6 Hz, 2H), 1.58 (s, 6H), 1.50 (s, 6H), 1.23 (t, J = 6.9 Hz, 3H) |
| 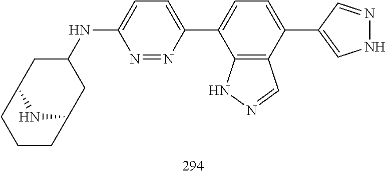 [SMILES: c1n[nH]cc1-c1ccc(-c2ccc(NC3C[C@H]4CCC[C@@H](C3)N4)nn2)c2[nH]ncc12] | 401 | 12.98-12.86 (s, 2H), 8.44 (s, 1H), 8.24 (s, 2H), 8.00 (d, J = 9.5 Hz, 1H), 7.83 (d, J = 7.7 Hz, 1H), 7.44 (d, J = 7.6 Hz, 1H), 6.98 (d, J = 9.5 Hz, 1H), 6.52 (d, J = 7.7 Hz, 1H), 4.95 (tq, J = 12.2, 6.2 Hz, 1H), 3.17 (s, 2H), 2.21- 2.11 (m, 2H), 2.01 (ddt, J = 19.3, 13.1, 6.3 Hz, 1H), 1.85 |
| | | (tt, J = 12.3, 5.6 Hz, 2H), |
| | | 1.69 (qd, J = 13.3, 11.8, 5.7 |
| | | Hz, 5H) |
| 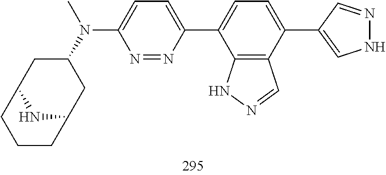 [SMILES: CN(c1ccc(-c2ccc(-c3cn[nH]c3)c3cn[nH]c23)nn1)[C@@H]1C[C@H]2CCC[C@@H](C1)N2] | 415 | 13.09 (s, 1H), 9.24 (s, 1H), 8.76 (s, 1H), 8.56 (s, 1H), 8.33 (d, J = 17.1 Hz, 2H), 7.96 (d, J = 7.6 Hz, 1H), 7.50 (d, J = 7.6 Hz, 1H), 5.76 (s, 1H), 3.78 (s, 2H), 3.05 (s, 3H), 2.51 (s, 3H), 1.95 (s, 6H), 1.77 (d, J = 13.0 Hz, 1H) |
| 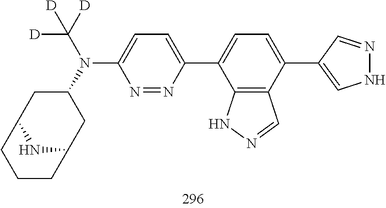 [SMILES: [2H]C([2H])([2H])N(c1ccc(-c2ccc(-c3cn[nH]c3)c3cn[nH]c23)nn1)[C@@H]1C[C@H]2CCC[C@@H](C1)N2] | 418 | 12.91 (s, 2H), 8.45 (s, 1H), 8.25 (s, 2H), 8.12 (d, J = 9.8 Hz, 1H), 7.86 (d, J = 7.7 Hz, 1H), 7.45 (d, J = 7.6 Hz, 1H), 7.18 (d, J = 9.7 Hz, 1H), 5.70 (tt, J = 12.2, 6.0 Hz, 1H), 3.23 (s, 2H), 2.06 (dtd, J = 24.8, 12.4, 5.8 Hz, 3H), 1.86 (td, J = 12.6, 6.1 Hz, 3H), 1.72 (td, J = 12.8, 12.3, 5.7 Hz, 5H) |
| 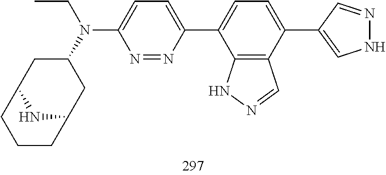 [SMILES: CCN(c1ccc(-c2ccc(-c3cn[nH]c3)c3cn[nH]c23)nn1)[C@@H]1C[C@H]2CCC[C@@H](C1)N2] | 429 | 13.18 (s, 1H), 13.07 (s, 1H), 8.51 (s, 1H), 8.33 (s, 2H), 8.13 (d, J = 9.8 Hz, 1H), 7.87 (d, J = 7.7 Hz, 1H), 7.47 (d, J = 7.6 Hz, 1H), 7.15 (d, J = 9.8 Hz, 1H), 5.75 (s, 1H), 3.52 (q, J = 6.8 Hz, 2H), 3.20 (s, 2H), 2.06 (s, 1H), 1.98 (td, J = 12.7, 5.4 Hz, 2H), 1.83 (dt, J = 12.7, 6.6 Hz, 2H), 1.71 |
| | | (ddt, J = 19.5, 13.8, 6.2 Hz, |
| | | 5H), 1.19 (t, J = 6.9 Hz, 3H) |
| 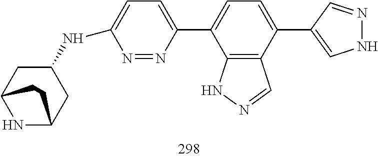 | 387 | 12.99 (s, 1H), 9.14 (s, 1H), 9.00 (s, 1H), 8.63 (s, 1H), 8.50 (s, 1H), 8.40 (s, 2H), 8.00 (d, J = 7.7 Hz, 1H), 7.67 (s, 1H), 7.56 (d, J = 7.7 Hz, 1H), 4.38 (s, 1H), 4.11 (d, J = 4.9 Hz, 2H), 2.26- 2.18 (m, 4H), 2.05-1.99 (m, 2H), 1.99-1.89 (m, 2H) |
| 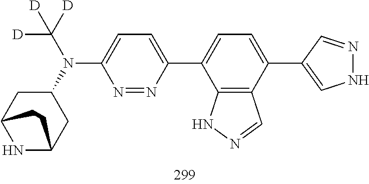 | 404 | 13.18 (s, 1H), 13.07 (s, 1H), 8.50 (s, 1H), 8.33 (s, 2H), 8.15 (d, J = 9.8 Hz, 1H), 7.89 (d, J = 7.7 Hz, 1H), 7.47 (d, J = 7.6 Hz, 1H), 7.24 (d, J = 9.8 Hz, 1H), 5.05 (s, 1H), 3.51 (s, 2H), 2.32 (m, 1H), 1.81 (td, J = 12.2, 3.0 Hz, 2H), 1.76 (s, 4H), 1.61-1.52 (m, 2H) |
| 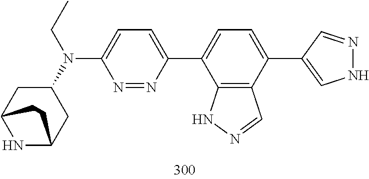 | 415 | 13.18 (s, 1H), 13.05 (s, 1H), 8.50 (s, 1H), 8.33 (s, 2H), 8.12 (d, J = 9.7 Hz, 1H), 7.87 (d, J = 7.7 Hz, 1H), 7.47 (d, J = 7.6 Hz, 1H), 7.19 (d, J = 9.8 Hz, 1H), 5.01 (s, 1H), 3.53 (d, J = 7.3 Hz, 4H), 1.84-1.77 (m, 1H), 1.76 (s, 6H), 1.64 (s, 2H), 1.17 (t, J = 6.9 Hz, 3H) |
| 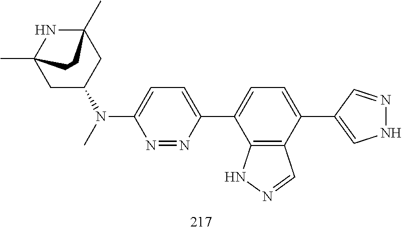 | 429 | 8.49 (s, 1H), 8.32 (s, 2H), 8.15 (d, J = 9.8 Hz, 1H), 7.89 (d, J = 7.7 Hz, 1H), 7.47 (d, J = 7.6 Hz, 1H), 7.25 (d, J = 9.8 Hz, 1H), 5.06 (s, 1H), 2.97 (s, 3H), 1.84 (d, J = 7.3 Hz, 2H), 1.64-1.50 (m, 6H), 1.19 (s, 6H) |
| 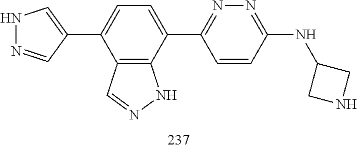 [SMILES: c1n[nH]cc1-c1ccc(-c2ccc(NC3CNC3)nn2)c2[nH]ncc12] | 333 | 13.02 (s, 1H), 9.08 (s, 2H), 8.57 (s, 2H), 8.35 (d, J = 17.2 Hz, 3H), 7.95 (d, J = 7.7 Hz, 1H), 7.61-7.48 (m, 1H), 7.29 (s, 1H), 4.92 (d, J = 7.1 Hz, 1H), 4.39-4.27 (m, 2H), 4.10 (t, J = 8.9 Hz, 2H) |
| 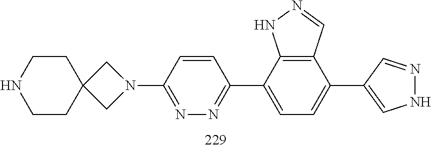 [SMILES: c1n[nH]cc1-c1ccc(-c2ccc(N3CC4(CCNCC4)C3)nn2)c2[nH]ncc12] | 387 | 13.04 (s, 1H), 8.92 (s, 2H), 8.63 (s, 1H), 8.58 (s, 1H), 8.40 (s, 2H), 8.03 (d, J = 7.6 Hz, 1H), 7.55 (d, J = 7.6 Hz, 1H), 7.49 (s, 1H), 4.17 (d, J = 3.1 Hz, 4H), 3.09 (s, 4H), 2.06 (t, J = 5.7 Hz, 4H) |
| 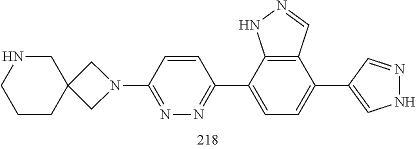 [SMILES: c1n[nH]cc1-c1ccc(-c2ccc(N3CC4(CCCNC4)C3)nn2)c2[nH]ncc12] | 387 | 13.03 (s, 1H), 9.35 (s, 2H), 8.62 (d, J = 19.4 Hz, 2H), 8.41 (s, 2H), 8.05 (d, J = 7.8 Hz, 1H), 7.62-7.53 (m, 2H), 4.35 (d, J = 9.9 Hz, 2H), 4.13 (d, J = 9.9 Hz, 2H), 3.3 (s, 2H), 2.99 (s, 2H), 1.97 (t, J = 5.8 Hz, 2H), 1.76 (s, 2H) |
| 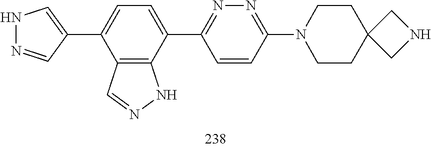 [SMILES: c1n[nH]cc1-c1ccc(-c2ccc(N3CCC4(CC3)CNC4)nn2)c2[nH]ncc12] | 387 | 13.04 (s, 1H), 9.04 (s, 2H), 8.62-8.56 (m, 1H), 8.38 (d, J = 2.5 Hz, 3H), 8.05-7.97 (m, 1H), 7.80 (s, 1H), 7.52 (dd, J = 7.5, 2.7 Hz, 1H), 3.84-3.73 (m, 8H), 1.94 (dt, J = 7.8, 4.3 Hz, 4H) |
| 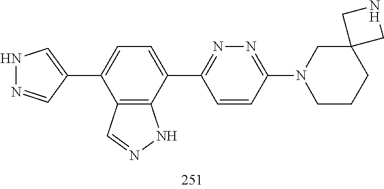 [SMILES: c1n[nH]cc1-c1ccc(-c2ccc(N3CCCC4(CNC4)C3)nn2)c2[nH]ncc12] | 387 | 13.08-13.19 (s, 2H), 8.51 (s, 1H), 8.34 (s, 2H), 8.19 (d, J = 9.9 Hz, 1H), 7.93 (d, J = 7.7 Hz, 1H), 7.52 (t, J = 10.5 Hz, 1H), 7.47 (d, J = 7.6 Hz, 1H), 3.87 (d, J = 4.6 Hz, 2H), 3.67-3.56 (m, 2H), 3.53 (s, 1H), 3.22 (s, 3H), 1.84-1.77 (m, 2H), 1.64- 1.58 (m, 2H) |
| 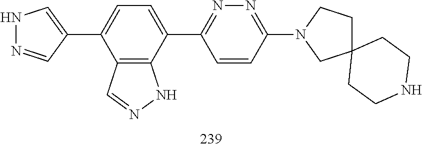 [SMILES: c1n[nH]cc1-c1ccc(-c2ccc(N3CCC4(CCNCC4)C3)nn2)c2[nH]ncc12] | 401 | 12.95 (s, 1H), 8.90 (s, 1H), 8.78 (s, 1H), 8.58 (s, 2H), 8.40 (s, 2H), 8.05 (d, J = 7.7 Hz, 1H), 7.68 (s, 1H), 7.55 (d, J = 7.7 Hz, 1H), 3.8 (m, 2H), 3.78 (t, J = 7.1 Hz, 2H), 3.17 (s, 2H), 3.09 (s, 2H), 2.07 (t, J = 7.1 Hz, 2H), 1.81 (q, J = 6.8, 6.1 Hz, 4H) |
| 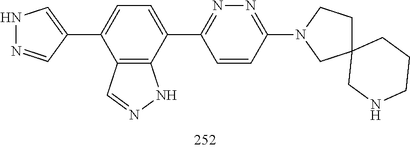 [SMILES: c1n[nH]cc1-c1ccc(-c2ccc(N3CCC4(CCCNC4)C3)nn2)c2[nH]ncc12] | 401 | 12.95 (s, 1H), 9.38 (s, 1H), 9.19 (s, 1H), 8.67 (d, J = 8.8 Hz, 2H), 8.41 (s, 2H), 8.07 (d, J = 7.7 Hz, 1H), 7.74 (d, J = 9.9 Hz, 1H), 7.57 (d, J = 7.7 Hz, 1H), 4.09 (s, 1H), 3.85-3.77 (m, 2H), 3.52 (d, J = 11.5 Hz, 1H), 3.12 (d, J = 5.9 Hz, 4H), 2.38-2.28 |
| | | (m, 1H), 2.01 (dt, J = 12.8, |
| | | 8.5 Hz, 1H), 1.79 (d, J = 7.9 |
| | | Hz, 4H) |
| 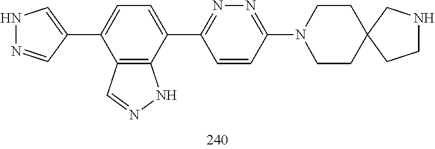 [SMILES: c1n[nH]cc1-c1ccc(-c2ccc(N3CCC4(CCNC4)CC3)nn2)c2[nH]ncc12] | 401 | 13.07 (s, 2H), 8.51 (s, 1H), 8.34 (s, 2H), 8.19 (dd, J = 9.9, 5.0 Hz, 1H), 7.94 (dd, J = 7.8, 2.1 Hz, 1H), 7.51- 7.44 (m, 2H), 3.87-3.72 (m, 1H), 3.72 (d, J = 5.8 Hz, 1H), 3.72-3.64 (m, 2H), 3.37 (d, J = 7.1 Hz, 1H), 3.22 (s, 1H), 2.86 (t, J = 7.1 |
| | | Hz, 1H), 2.66 (s, 1H), 1.81 |
| | | (t, J = 7.1 Hz, 1H), 1.60 (q, |
| | | J = 6.1, 5.2 Hz, 5H) |
| 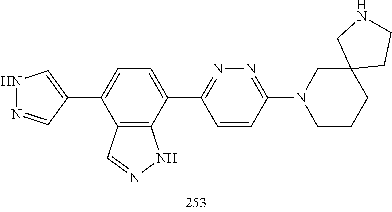 [SMILES: c1n[nH]cc1-c1ccc(-c2ccc(N3CCCC4(CCNC4)C3)nn2)c2[nH]ncc12] | 401 | 13.18-13.07 (d, J = 14.0 Hz, 2H), 8.51 (s, 1H), 8.33 (s, 2H), 8.16 (d, J = 9.8 Hz, 1H), 7.93 (d, J = 7.7 Hz, 1H), 7.51-7.44 (m, 2H), 3.80 (d, J = 13.1 Hz, 1H), 3.67 (d, J = 13.9 Hz, 1H), 3.58 (d, J = 12.8 Hz, 2H), 3.50 (d, J = 12.9 Hz, 2H), 2.91 (ddd, J = 10.7, 7.9, 6.0 Hz, 1H), 2.83-2.69 (m, 1H), 1.64 (d, J = 4.9 Hz, 4H), 1.42 (ddd, J = 12.6, 8.3, |
| | | 6.0 Hz, 2H) |
| 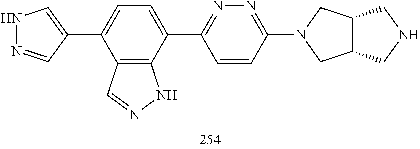 [SMILES: c1n[nH]cc1-c1ccc(-c2ccc(N3C[C@H]4CNC[C@H]4C3)nn2)c2[nH]ncc12] | 373 | 13.08 (s, 2H), 8.51 (s, 1H), 8.20 (d, J = 9.5 Hz, 3H), 7.92 (d, J = 7.7 Hz, 1H), 7.47 (d, J = 7.6 Hz, 1H), 7.10 (t, J = 9.2 Hz, 1H), 3.80 (dd, J = 10.6, 7.2 Hz, 2H), 3.59 (s, 1H), 3.53-3.38 (m, 2H), 3.07 (s, 1H), 2.98 (dd, J = 10.4, 6.7 Hz, 3H), 2.70 |
| | | (dd, J = 10.4, 2.5 Hz, 1H) |
| 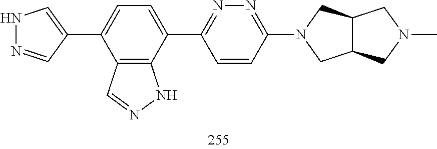 [SMILES: CN1C[C@@H]2CN(c3ccc(-c4ccc(-c5cn[nH]c5)c5cn[nH]c45)nn3)C[C@@H]2C1] | 387 | 13.18 (s, 1H), 13.07 (s, 1H), 8.51 (s, 1H), 8.36 (s, 1H), 8.20 (d, J = 9.6 Hz, 2H), 7.92 (d, J = 7.7 Hz, 1H), 7.47 (d, J = 7.6 Hz, 1H), 7.12 (d, J = 9.6 Hz, 1H), 3.78 (dd, J = 10.7, 8.0 Hz, 2H), 3.48 (dd, J = 10.8, 3.1 Hz, 2H), 2.99 (q, J = 4.4, 3.9 |
| | | Hz, 2H), 2.60-2.51 (m, |
| | | 4H), 2.25 (s, 3H) |
| 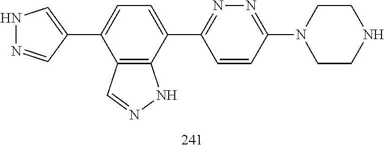 [SMILES: c1n[nH]cc1-c1ccc(-c2ccc(N3CCNCC3)nn2)c2[nH]ncc12] | 347 | 13.10 (s, 1H), 9.29 (s, 2H), 8.60-8.55 (m, 1H), 8.41 (d, J = 4.4 Hz, 3H), 8.01 (d, J = 7.7 Hz, 1H), 7.70-7.62 (m, 1H), 7.51 (d, J = 7.6 Hz, 1H), 4.02-3.95 (m, 4H), 3.29 (t, J = 5.2 Hz, 4H) |
| 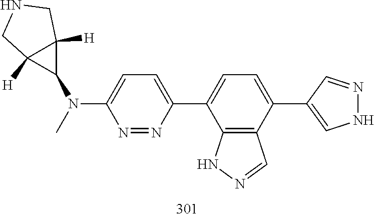 [SMILES: CN(c1ccc(-c2ccc(-c3cn[nH]c3)c3cn[nH]c23)nn1)[C@H]1[C@@H]2CNC[C@@H]21] | 373 | 9.75 (s, 1H), 9.51 (s, 1H), 8.64 (s, 1H), 8.58 (d, J = 9.9 Hz, 1H), 8.41 (s, 2H), 8.06 (d, J = 7.8 Hz, 1H), 7.84 (d, J = 9.8 Hz, 1H), 7.56 (d, J = 7.6 Hz, 1H), 3.71 (dd, J = 11.7, 6.0 Hz, 2H), 3.43 (d, J = 7.3 Hz, 2H), 3.32 (s, 3H), 3.19 (d, J = 2.5 Hz, 1H), 2.35 (d, J = 2.8 Hz, 2H) |
| 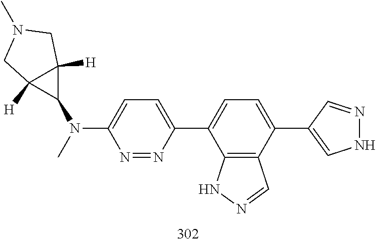 [SMILES: CN1C[C@@H]2[C@H](C1)[C@H]2N(C)c1ccc(-c2ccc(-c3cn[nH]c3)c3cn[nH]c23)nn1] | 387 | 13.11 (s, 1H), 10.65 (s, 1H), 8.42 (d, J = 12.5 Hz, 1H), 8.37 (s, 3H), 8.00 (dd, J = 7.8, 4.3 Hz, 1H), 7.60-7.49 (m, 2H), 3.91 (dd, J = 11.5, 5.1 Hz, 2H), 3.42 (t, J = 9.6 Hz, 2H), 3.28 (s, 4H), 2.83 (d, J = 4.6 Hz, 3H), 2.31 (s, 2H) |
| 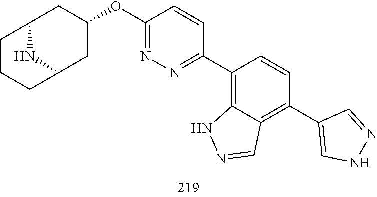 [SMILES: c1n[nH]cc1-c1ccc(-c2ccc(O[C@@H]3C[C@H]4CCC[C@@H](C3)N4)nn2)c2[nH]ncc12] | 402 | 13.20 (s, 1H), 9.02 (s, 1H), 8.93 (s, 1H), 8.56 (s, 1H), 8.47 (d, J = 9.5 Hz, 1H), 8.38 (s, 2H), 8.03 (d, J = 7.8 Hz, 1H), 7.52 (d, J = 7.6 Hz, 1H), 7.40 (d, J = 9.4 Hz, 1H), 6.14 (dq, J = 11.3, 6.1, 5.6 Hz, 1H), 3.8 (s, 2H), 2.61- 2.53 (m, 2H), 2.22-2.10 (m, 2H), 2.04 (s, 2H), 1.81 (s, 4H) |
| 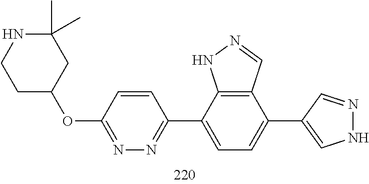 [SMILES: CC1(C)CC(Oc2ccc(-c3ccc(-c4cn[nH]c4)c4cn[nH]c34)nn2)CCN1] | 390 | 13.1 (s, 1H), 9.09 (s, 2H), 8.57 (s, 1H), 8.48 (d, J = 9.5 Hz, 1H), 8.38 (s, 2H), 8.04 (d, J = 7.8 Hz, 1H), 7.53 (d, J = 7.6 Hz, 1H), 7.40 (d, J = 9.4 Hz, 1H), 5.65 (dt, J = 9.5, 5.0 Hz, 1H), 3.34 (s, 2H), 2.35 (s, 1H), 2.28 (dd, J = 13.7, 4.1 Hz, 1H), 1.96 (s, 2H), 1.44 (d, J = 4.6 Hz, 6H) |
| 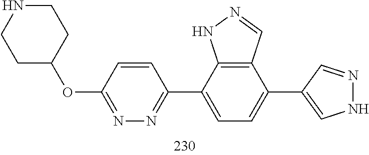 [SMILES: c1n[nH]cc1-c1ccc(-c2ccc(OC3CCNCC3)nn2)c2[nH]ncc12] | 398 | 13.21 (s, 1H), 13.15 (s, 1H), 8.55 (s, 2H), 8.44 (d, J = 9.4 Hz, 1H), 8.21 (s, 1H), 8.02 (d, J = 7.7 Hz, 1H), 7.51 (d, J = 7.7 Hz, 1H), 7.36 (d, J = 9.4 Hz, 1H), 5.45-5.38 (m, 1H), 3.11 (d, J = 12.3 Hz, 2H), 2.80 (d, J = 11.4 Hz, 2H), 2.16 (d, J = 12.7 Hz, 2H), 1.72 (d, J = 11.1 Hz, 2H) |
| 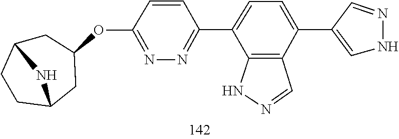 [SMILES: c1n[nH]cc1-c1ccc(-c2ccc(O[C@@H]3C[C@H]4CC[C@@H](C3)N4)nn2)c2[nH]ncc12] | 424 | 13.19 (d, J = 17.2 Hz, 2H), 8.55 (s, 1H), 8.40 (d, J = 9.4 Hz, 3H), 8.00 (d, J = 7.7 Hz, 1H), 7.51 (d, J = 7.6 Hz, 1H), 7.30 (d, J = 9.4 Hz, 1H), 5.62 (tt, J = 11.1, 5.7 Hz, 1H), 3.53 (s, 2H), 2.26- 2.16 (m, 2H), 1.77-1.69 (m, 4H), 1.63 (t, J = 10.2 Hz, 2H) |
| 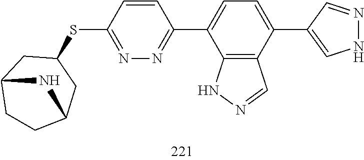 [SMILES: c1n[nH]cc1-c1ccc(-c2ccc(S[C@@H]3C[C@H]4CC[C@@H](C3)N4)nn2)c2[nH]ncc12] | 404 | 13.23 (d, J = 11.0 Hz, 2H), 8.58 (d, J = 4.8 Hz, 1H), 8.23 (s, 3H), 8.07 (dd, J = 7.8, 3.4 Hz, 1H), 7.76-7.63 (m, 1H), 7.54 (dd, J = 7.8, 4.8 Hz, 1H), 4.60 (t, J = 7.3 Hz, 1H), 3.59 (s, 2H), 2.42 (s, 2H), 2.09 (t, J = 6.8 Hz, 2H), 1.88 (d, J = 14.9 Hz, 4H) |
| 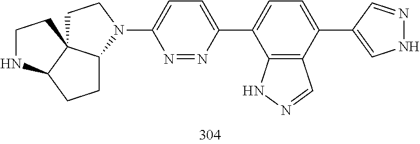 [SMILES: c1n[nH]cc1-c1ccc(-c2ccc(N3CC[C@@]45CCN[C@@H]4CC[C@@H]35)nn2)c2[nH]ncc12] | 430 | 13.19 (s, 1H), 13.07 (s, 1H), 8.50 (s, 1H), 8.33 (s, 2H), 8.18 (t, J = 10.1 Hz, 1H), 7.91 (dd, J = 7.8, 4.7 Hz, 1H), 7.47 (d, J = 7.5 Hz, 1H), 7.10 (dd, J = 11.8, 9.5 Hz, 1H), 4.07 (d, J = 4.5 Hz, 1H), 3.90 (s, 1H), 3.64 (s, 1H), 3.61-3.44 (m, 1H), |
| | | 2.91 (dt, J = 11.4, 6.0 Hz, |
| | | 1H), 2.86-2.77 (m, 1H), |
| | | 2.53 (s, 2H), 2.17 (d, J = 6.7 |
| | | Hz, 1H), 2.12-1.99 (m, |
| | | 1H), 1.97-1.86 (m, 1H), |
| | | 1.80 (dt, J = 12.1, 6.0 Hz, |
| | | 1H), 1.66 (tt, J = 12.3, 6.2 |
| | | Hz, 1H), 1.24 (s, 1H) |
| 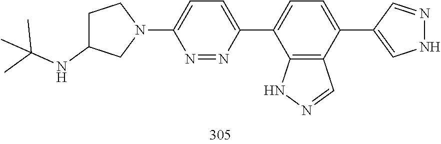 [SMILES: CC(C)(C)NC1CCN(c2ccc(-c3ccc(-c4cn[nH]c4)c4cn[nH]c34)nn2)C1] | 403 | 13.10 (s, 1H), 9.10 (s, 1H), 8.94 (d, J = 12.7 Hz, 1H), 8.57 (s, 1H), 8.37 (s, 3H), 8.00 (d, J = 7.7 Hz, 1H), 7.51 (d, J = 7.6 Hz, 1H), 7.42 (s, 1H), 4.21 (s, 1H), 4.08 (dd, J = 11.4, 6.8 Hz, 1H), 3.84 (dd, J = 11.2, 5.7 Hz, 2H), 3.81 (s, 1H), 2.68 (s, 1H), 2.33 (s, 1H), 1.40 (s, 9H) |
| 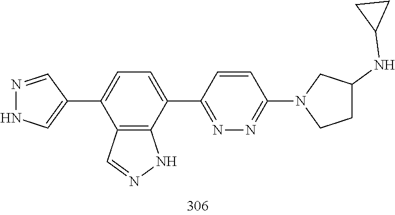 [SMILES: c1n[nH]cc1-c1ccc(-c2ccc(N3CCC(NC4CC4)C3)nn2)c2[nH]ncc12] | 387 | 13.18 (s, 1H), 13.09 (s, 1H), 8.50 (s, 2H), 8.18 (d, J = 9.6 Hz, 2H), 7.91 (d, J = 7.7 Hz, 1H), 7.47 (d, J = 7.6 Hz, 1H), 7.05 (d, J = 9.6 Hz, 1H), 3.75 (dd, J = 10.5, 5.9 Hz, 1H), 3.66 (d, J = 7.5 Hz, 1H), 3.55 (dq, J = 11.3, 6.1, 5.5 Hz, 2H), 3.42 (d, 1H), 2.15 (q, J = 6.2 Hz, 2H), 2.00-1.90 (m, 1H), 0.43 (d, J = 6.8 Hz, 2H), 0.32-0.21 (m, 2H) |

### Example 69: Exemplary Splicing Assay for Monitoring Expression Levels of Splice Variants

To set up 10 uL RT-qPCR reactions, cell lysates were transferred to 384-well qPCR plates containing the master mix according to the table below. The plates were sealed, gently vortexed, and spun down before the run. The volumes were adjusted accordingly in some instances where the reaction was carried in 20 uL. The table below summarizes the components of the RT-qPCR reactions:

| Component | 1X |
|---|---|
| Taqman 1-step RT-qPCR mix (4X) | 2.5 |
| 20X AJ Primers + Probe (FAM) | 0.5 |
| 20X CJ Primers + Probe (CY5) | 0.5 |
| 20X PPIA Control (VIC) | 0.5 |
| Cell lysate (1X) | 1-2 |
| H₂O | 4-5 |
| Total volume | 10 |

The RT-qPCR reaction was performed using a QuantStudio (ThermoFisher) under the following fast cycling conditions. All samples and standards were analyzed at least in duplicate. In some instances, bulk room temperature (RT) step of 5-10 minutes was completed for all plates before proceeding with qPCR. The table below summarizes the PCR cycle:

| Step | # cycles | Temp. | Time | |
|---|---|---|---|---|
| RT step | 1 | 50° C. | 5 | min |
| RT inactivation/initial | 1 | 95° C. | 20 | sec |
| denaturation | | | | |
| Amplification | 40 | 95° C. | 3 | sec |
| | | 60° C. | 30 | sec |

The data analysis was performed by first determining the ΔCt vs the housekeeper gene. This ΔCt was then normalized against the DMSO control (ΔΔCt) and converted to RQ (relative quantification) using the 2\hat{ }(−ΔΔCt) equation. The RQ were then converted to a percentage response by arbitrarily setting an assay window of 3.5 ΔCt for HTT-CJ and an assay window of 9 ΔCt for HTT-AJ. These assay windows correspond to the maximal modulation observed at high concentration of the most active compounds. The percentage response was then fitted to the 4 parametric logistic equation to evaluate the concentration dependence of compound treatment. The increase in AJ mRNA is reported as AC₅₀ (compound concentration having 50% response in AJ increase) while the decrease in CJ mRNA levels is reported as IC₅₀ (compound concentration having 50% response in CJ decrease).

A summary of these results is illustrated in Table 7, wherein “A” represents an AC₅₀ of less than 500 nM; “B” represents an AC₅₀ of between 500 nM and 5 μM; and “C” represents an AC₅₀ of greater than 5 μM.

TABLE 7
Modulation of RNA Splicing by Exemplary Compounds

| | HTT AJ | HTT CJ |
|---|---|---|
| Compound | AC₅₀ | AC₅₀ |
| No. | (nM) | (nM) |
| 104 | A | — |
| 105 | A | A |
| 108 | C | C |
| 111 | C | C |
| 112 | B | A |
| 113 | C | B |
| 115 | C | — |
| 117 | C | C |
| 118 | B | B |
| 119 | C | B |
| 121 | C | — |
| 131 | C | C |
| 132 | A | A |
| 137 | B | B |
| 138 | B | A |
| 141 | A | A |
| 142 | B | A |
| 143 | B | A |
| 146 | A | A |
| 149 | A | A |
| 150 | B | B |
| 157 | C | — |
| 158 | C | C |
| 159 | C | C |
| 160 | C | C |
| 169 | B | A |
| 170 | C | B |
| 171 | B | A |
| 181 | C | C |
| 182 | B | A |
| 183 | C | C |
| 184 | B | A |
| 185 | C | C |
| 186 | A | A |
| 187 | C | C |
| 188 | C | C |

Additional results are provided below in Table 8, wherein “A” represents an AC₅₀/IC₅₀ of less than 100 nM; “B” represents an AC₅₀/IC₅₀ of between 100 nM and 1 μM; and “C” represents an AC₅₀/IC₅₀ of between 1 μM and 10 μM; and “D” represents an AC₅₀/IC₅₀ of greater than 10 μM.

TABLE 8
Modulation of RNA Splicing by Exemplary Compounds

| | HTT AJ | HTT CJ |
|---|---|---|
| Compound | AC₅₀ | AC₅₀ |
| No. | (nM) | (nM) |
| 170 | C | C |
| 174 | C | C |
| 175 | C | D |
| 176 | C | D |
| 177 | D | D |
| 178 | D | D |
| 179 | B | B |
| 180 | B | B |
| 181 | D | D |
| 182 | C | C |
| 183 | B | B |
| 184 | A | B |
| 185 | C | D |
| 186 | A | A |
| 187 | D | D |
| 188 | D | D |
| 189 | D | D |
| 190 | D | D |
| 191 | D | D |
| 192 | D | D |
| 193 | D | D |
| 194 | D | C |
| 196 | D | D |
| 197 | C | C |
| 198 | D | D |
| 200 | D | D |
| 201 | C | C |
| 202 | A | A |
| 203 | B | B |
| 204 | A | A |
| 205 | D | D |
| 206 | D | C |
| 209 | D | D |
| 210 | A | A |
| 213 | A | A |
| 214 | C | D |
| 215 | A | A |
| 216 | A | A |
| 218 | D | D |
| 219 | B | B |
| 220 | D | C |
| 221 | D | D |
| 222 | C | C |
| 223 | C | C |
| 224 | D | D |
| 225 | C | C |
| 226 | A | A |
| 227 | A | A |
| 228 | B | A |
| 229 | C | C |
| 230 | D | D |
| 231 | B | B |
| 232 | B | C |
| 233 | B | B |
| 234 | D | D |
| 235 | D | D |
| 237 | D | D |
| 238 | D | D |
| 239 | D | D |
| 240 | D | D |
| 241 | C | D |
| 242 | D | D |
| 243 | C | C |
| 251 | D | D |
| 252 | D | D |
| 253 | D | D |
| 254 | D | D |
| 255 | D | D |
| 263 | D | D |
| 264 | D | D |
| 265 | D | D |
| 266 | D | C |
| 267 | D | D |
| 273 | D | D |
| 274 | D | D |
| 275 | C | B |
| 276 | C | C |
| 277 | D | D |
| 278 | B | A |
| 279 | C | C |
| 280 | B | B |
| 281 | D | D |
| 282 | D | C |

Additional studies were carried out for a larger panel of genes using the protocol provided above. The junction between flanking upstream and downstream exons was used to design canonical junction qPCR assays. At least one of the forward primer, reverse primer or the CY5-labeled 5′ nuclease probe (with 3′ quencher such as ZEN/Iowa Black FQ) was designed to overlap with the exon junction to capture the CJ mRNA transcript. BLAST was used to confirm the specificity of the probeset and parameters such as melting temperature, GC content, amplicon size, and primer dimer formation are considered during their design. Data for the decrease in CJ mRNA levels for three exemplary genes (HTT, SMN2, and Target C) analyzed in this panel are reported as IC₅₀ (compound concentration having 50% response in CJ decrease).

A summary of the results from the panel is illustrated in Table 9, wherein “A” represents an IC₅₀ of less than 100 nM; “B” represents an IC₅₀ of between 100 nM and 1 μM; and “C” represents an IC₅₀ of between 1 μM and 10 μM; and “D” represents an IC₅₀ of greater than 10

TABLE 9
Modulation of RNA Splicing by Exemplary Compounds

| Compound No. | HTT | SMN2 | Target C |
|---|---|---|---|
| 170 | C | B | D |
| 174 | C | B | D |
| 175 | D | C | D |
| 176 | D | D | D |
| 177 | D | C | D |
| 178 | D | D | D |
| 179 | B | B | B |
| 180 | B | B | B |
| 181 | D | A | D |
| 182 | C | B | C |
| 183 | B | B | B |
| 184 | B | A | B |
| 185 | D | B | D |
| 186 | A | A | B |
| 187 | D | C | D |
| 188 | D | D | D |
| 189 | D | D | D |
| 190 | D | D | D |
| 191 | D | C | D |
| 192 | D | D | D |
| 193 | D | D | D |
| 194 | C | B | D |
| 196 | D | D | D |
| 197 | C | C | D |
| 198 | D | B | D |
| 200 | D | D | D |
| 201 | C | B | C |
| 202 | A | A | B |
| 203 | B | A | B |
| 204 | A | A | A |
| 205 | D | C | D |
| 206 | C | A | D |
| 209 | D | B | D |
| 210 | A | A | A |
| 211 | B | B | B |
| 213 | A | A | A |
| 214 | D | B | D |
| 215 | A | A | A |
| 216 | A | A | B |
| 218 | D | D | D |
| 219 | B | A | B |
| 220 | C | B | D |
| 221 | D | D | D |
| 222 | C | B | C |
| 223 | C | B | C |
| 224 | D | C | D |
| 225 | C | B | C |
| 226 | A | A | A |
| 227 | A | A | A |
| 228 | A | A | A |
| 229 | C | C | C |
| 230 | D | C | D |
| 232 | C | B | C |

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A compound of Formula (I-d): 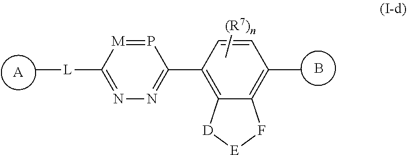
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
L is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴;
M and P are each independently C(R²) or N;
D, E, and F are each independently C(R^{5d}), C(R^{5d})(R^{5e}), N, N(R^{5f}), S, or O, wherein the bonds between the atoms in the ring comprising D, E, and F may be a single bonds or double bonds as valency permits;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁸; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R′;
each R² is independently hydrogen, halo, cyano, C₁-C₆-alkyl, C₂-C₆-alkenyl, C₂-C₆-alkynyl, or —OR^{A};
each R³ is independently hydrogen, C₁-C₆-alkyl, C₁-C₆-heteroalkyl, C₁-C₆-haloalkyl, cycloalkyl or heterocyclyl; wherein each alkyl, heteroalkyl, haloalkyl, cycloalkyl, and heterocyclyl is optionally substituted with one or more R¹²;
each R⁴ is C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
R^{5d} and R^{5c} are each independently hydrogen, halo, or C₁-C₆ alkyl; or
R^{5d} and R^{5c} are taken together to form an oxo group;
R^{5f} is hydrogen, halo, or C₁-C₆ alkyl;
each R⁷ is independently C₁-C₆-alkyl, C₂-C₆-alkenyl, C₂-C₆-alkynyl, C₁-C₆-heteroalkyl, C₁-C₆-haloalkyl, halo, oxo, cyano, NR^{B}C(O)R^{D}, —C(O)NR^{B}R^{C}, —C(O)R^{D}, or —SR^{E}, wherein alkyl, alkenyl, alkynyl, heteroalkyl, and haloalkyl are optionally substituted with one or more R⁹; or
two R⁷ groups, together with the atoms to which they are attached (e.g., X or Y), form a 4-7-membered cycloalkyl, heterocyclyl, aryl, or heteroaryl, wherein each cycloalkyl, heterocyclyl, aryl, and heteroaryl is optionally substituted with one or more R⁹;
R⁸ and R⁹ are each independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R¹¹;
each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₁-C₆-heteroalkyl, cycloalkyl, heterocyclyl, —OR^{A}; or
R^{B} and R^{C} together with the atom to which they are attached form a 3-7-membered heterocyclyl ring optionally substituted with one or more R¹⁰;
each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R¹⁰ is independently C₁-C₆-alkyl or halo;
each R¹¹ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};
each R¹² is independently deuterium, C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};
n is 0, 1, 2, 3, or 4; and
x is 0, 1, or 2.

2. The compound of claim 1, wherein A is a monocyclic or bicyclic heterocyclyl.

3. The compound of any one of the preceding claims, wherein A is a nitrogen-containing heterocyclyl.

4. The compound of any one of the preceding claims, wherein A is selected from

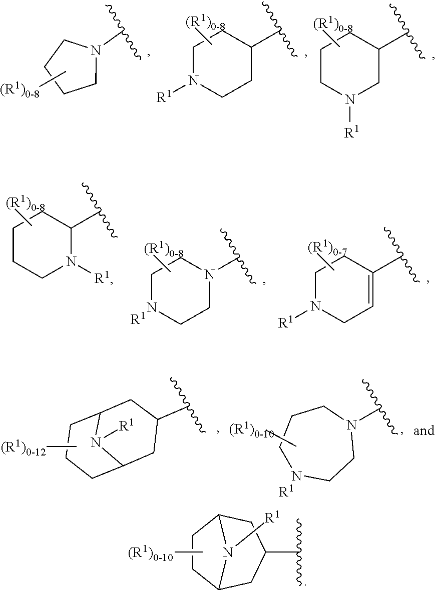

5. The compound of any one of the preceding claims, wherein A is selected from

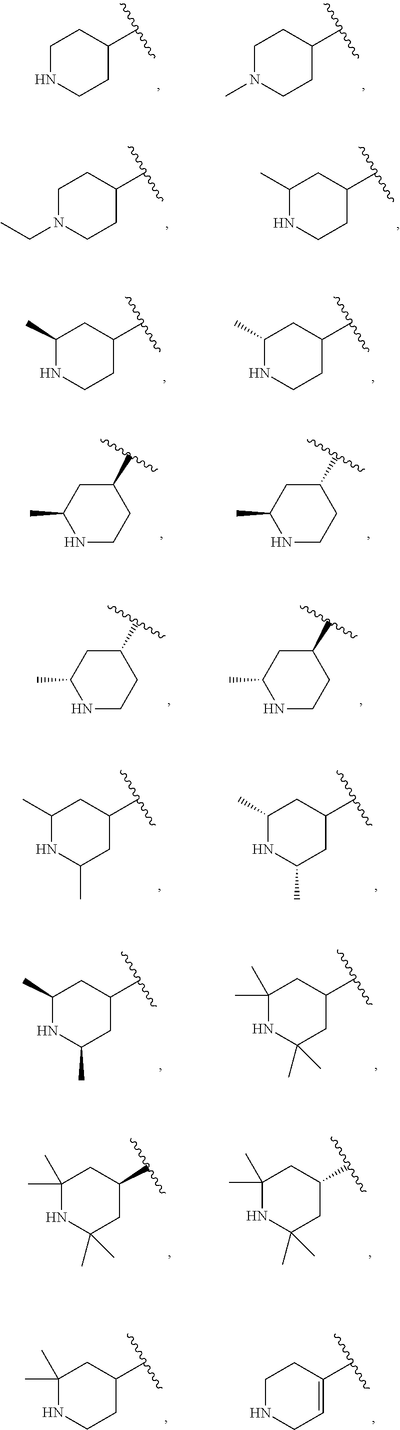

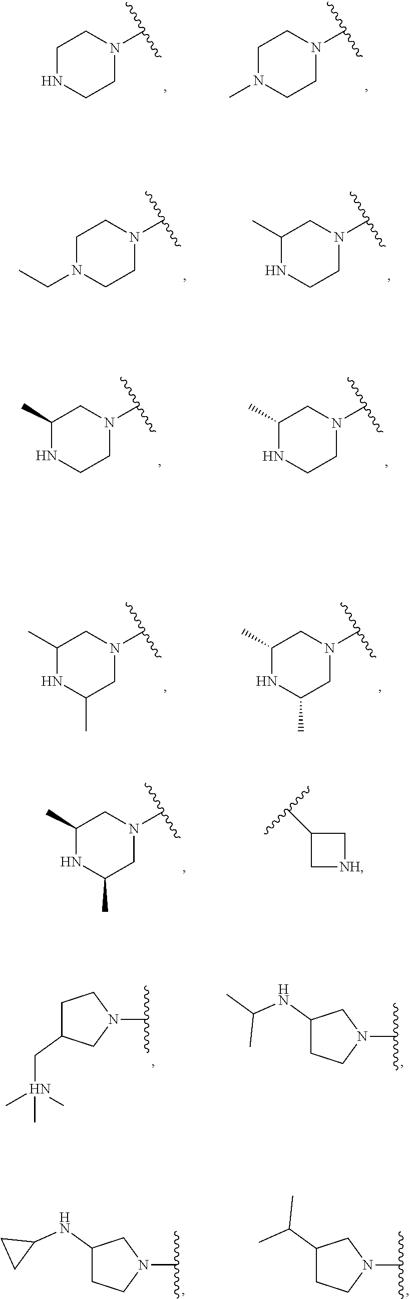

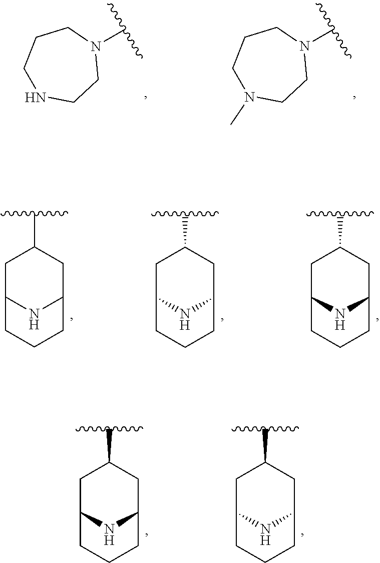

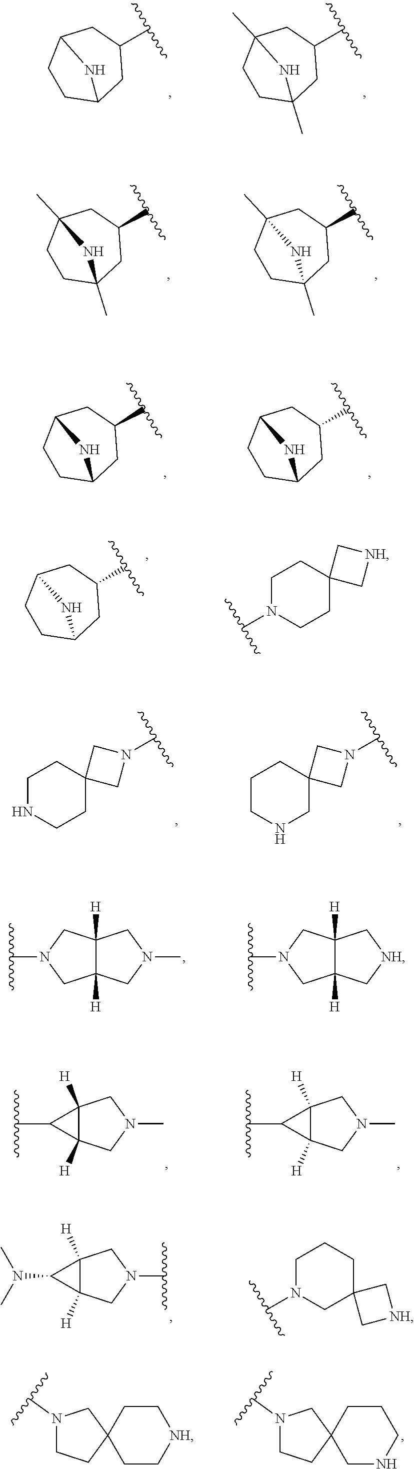

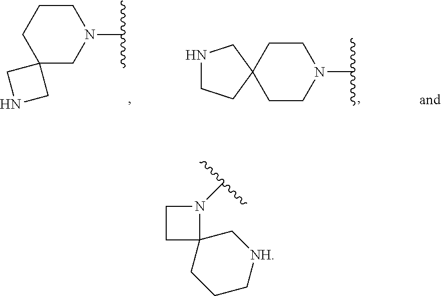 [SMILES: *N1CCCC2(CNC2)C1 ; *N1CCC2(CCNC2)CC1 ; *N1CCC12CCCNC2]

6. The compound of any one of the preceding claims, wherein A is selected from

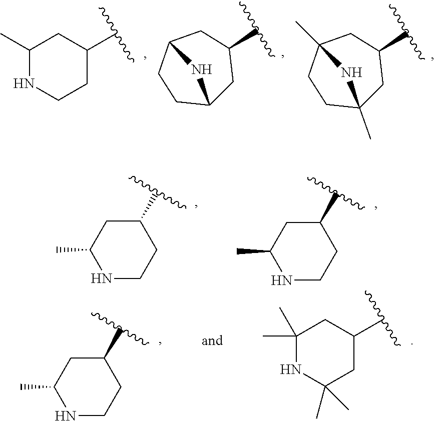

7. The compound of any one of the preceding claims, wherein B is selected from

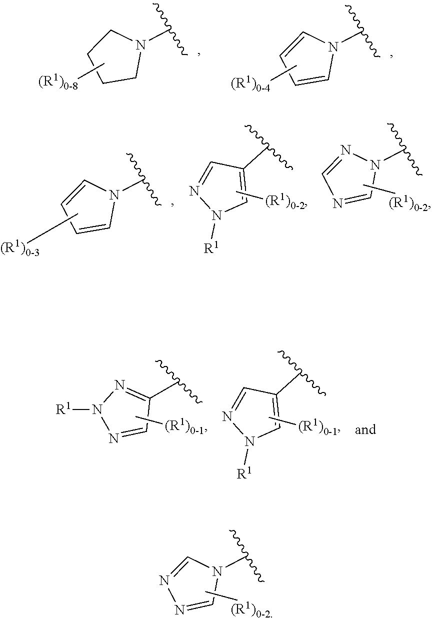

8. The compound of any one of the preceding claims, wherein B is selected from

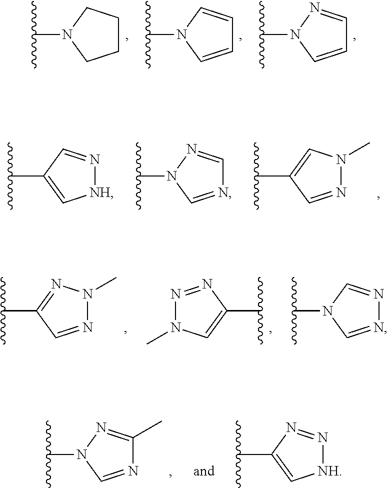

9. The compound of any one of the preceding claims, wherein B is selected from

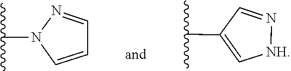 [SMILES: *n1cccn1 ; *c1cn[nH]c1]

10. The compound of any one of the preceding claims, wherein L is —O— or —N(R³)—.

11. The compound of any one of the preceding claims, wherein L is —N(R³)— and R³ is hydrogen, C₁-C₆-alkyl, or cycloalkyl, wherein each alkyl and heteroalkyl are each optionally substituted with one or more R¹².

12. The compound of any one of the preceding claims, wherein L is —N(CH₃)— or —N(H)—.

13. The compound of any one of the preceding claims, wherein L is —N(CH₃)—.

14. The compound of any one of the preceding claims, wherein 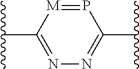
is selected from

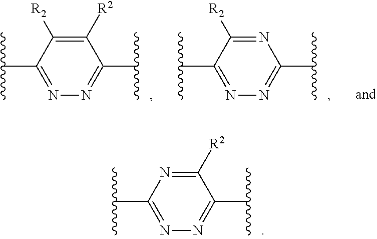

15. The compound of any one of the preceding claims, wherein 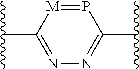
is

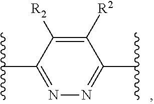

16. The compound of any one of the preceding claims, wherein each of M and P is independently C(R²) (e.g., CH).

17. The compound of any one of the preceding claims, wherein one of M and P is independently C(R²) (e.g., CH) and the other of M and P is independently N.

18. The compound of any one of the preceding claims, wherein M is CH and P is N.

19. The compound of any one of the preceding claims, wherein one of D, E, and F is independently N or N(R^{5f}).

20. The compound of any one of the preceding claims, wherein two of D, E, and F is independently N or N(R^{5f}).

21. The compound of any one of the preceding claims, wherein D is N, N(R^{5S}), or S.

22. The compound of any one of the preceding claims, wherein D is N or N(R^{5S}) (e.g., NH).

23. The compound of any one of the preceding claims, wherein each of D and E is independently N or N(R^{5S}).

24. The compound of any one of the preceding claims, wherein F is C(R^{5d}) or C(R^{5d})(R^{5e}).

25. The compound of any one of claims 1-18, wherein 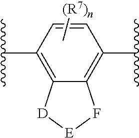
is selected from

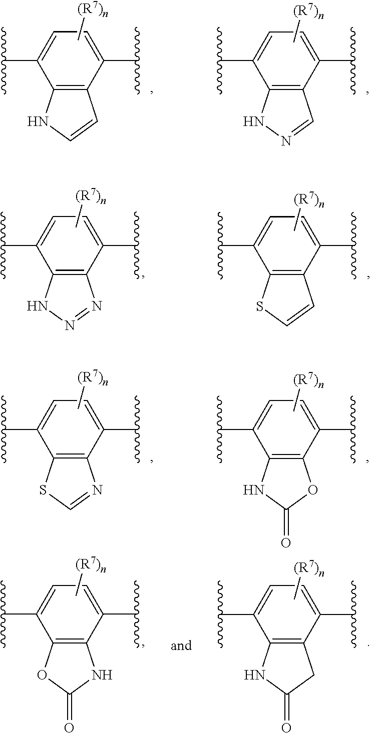

26. The compound of claim 25, wherein 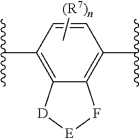
is selected from

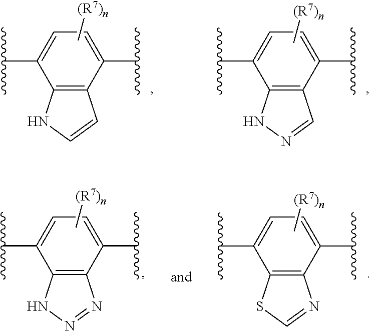

27. The compound of claim 26, wherein 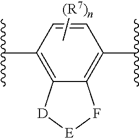
is

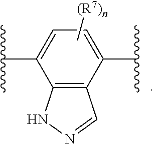

28. The compound of any one of the preceding claims, wherein the compound is a compound of Formula (I-e): 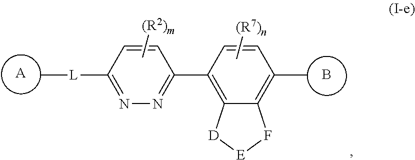
or a pharmaceutically acceptable salt, solvate, hydrate, tautomer, or stereoisomer thereof, wherein A, B, L, D, E, F, R², R⁷, m, n, and subvariables thereof are as described in claim 1.

29. The compound of any one of the preceding claims, wherein the compound is a compound of Formula (I-f): 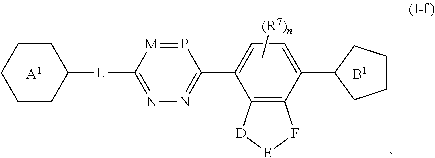
or a pharmaceutically acceptable salt, solvate, hydrate, tautomer, or stereoisomer thereof, wherein:
A¹ is 6-membered cycloalkyl, heterocyclyl, aryl, or heteroaryl, each of which is optionally substituted with one or more R¹;
B¹ is 5-membered cycloalkyl, heterocyclyl, aryl, or heteroaryl, each of which is optionally substituted with one or more R¹; and
L, M, P, D, E, F, R¹, R⁷, n, and subvariables thereof are as described in claim 1.

30. The compound of any one of the preceding claims, wherein the compound is a compound of Formula (I-g): 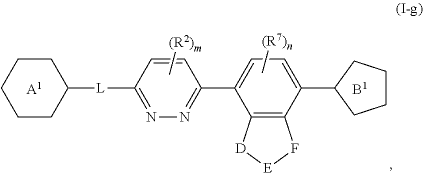
or a pharmaceutically acceptable salt, solvate, hydrate, tautomer, or stereoisomer thereof, wherein:
A¹ is 6-membered cycloalkyl, heterocyclyl, aryl, or heteroaryl, each of which is optionally substituted with one or more R¹;
B¹ is 5-membered cycloalkyl, heterocyclyl, aryl, or heteroaryl, each of which is optionally substituted with one or more R¹; and
L, D, E, F, R¹, R², R⁷, m, n, and subvariables thereof are as described in claim 1.

31. The compound of any one of the preceding claims, wherein the compound is a compound of Formula (I-h): 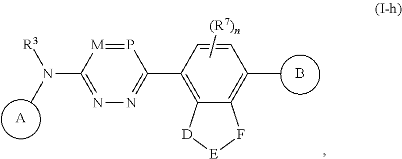
or a pharmaceutically acceptable salt, solvate, hydrate, tautomer, or stereoisomer thereof, wherein A, B, M, P, D, E, F, R³, R⁷, n, and subvariables thereof are as described in claim 1.

32. The compound of any one of the preceding claims, wherein the compound is a compound of Formula (I-k): 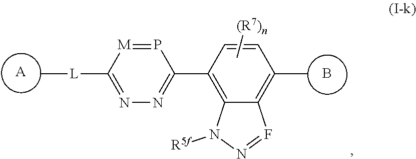
or a pharmaceutically acceptable salt, solvate, hydrate, tautomer, or stereoisomer thereof, wherein A, B, M, P, L, F, R^{5f}, R⁷, n, and subvariables thereof are as described in claim 1.

33. The compound of any one of the preceding claims, wherein the compound is a compound of Formula (I-n): 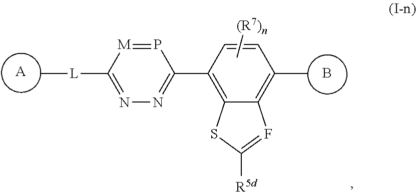
or a pharmaceutically acceptable salt, solvate, hydrate, tautomer, or stereoisomer thereof, wherein A, B, M, P, L, F, R^{5d}, R⁷, n, and subvariables thereof are as described in claim 1.

34. The compound of claim 1, wherein:
A is a monocyclic, bicyclic, or tricyclic heterocyclyl;
B is a monocyclic heterocyclyl or heteroaryl;
L is absent, —N(R³)—, or —O—;
M is CH;
P is CH or N;
D is N(R^{5f}), C(R^{5d}), or S;
E and F is each independently N, NH, C(R^{5d}), C(R^{5d})(R^{5e});
R⁷ is halo; and
m is 0 or 1.

35. The compound of any one of the preceding claims, wherein the compound is selected from any one of the compounds shown in Table 1 or a pharmaceutically acceptable salt, solvate, hydrate, tautomer, or stereoisomer thereof.

36. A pharmaceutical composition comprising a compound of any one of the preceding claims and a pharmaceutically acceptable excipient.

37. The compound of any one of claims 1-35, or the pharmaceutical composition of claim 36, wherein the compound alters a target nucleic acid (e.g., an RNA, e.g., a pre-mRNA).

38. The compound of any one of claims 1-35, or the pharmaceutical composition of claim 36, wherein the compound binds to a target nucleic acid (e.g., an RNA, e.g., a pre-mRNA).

39. The compound of any one of claims 1-35, or the pharmaceutical composition of claim 36, wherein the compound stabilizes a target nucleic acid (e.g., an RNA, e.g., a pre-mRNA).

40. The compound of any one of claims 1-35, or the pharmaceutical composition of claim 36, wherein the compound increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

41. The compound of any one of claims 1-35, or the pharmaceutical composition of claim 36, wherein the compound decreases splicing at splice site on a target nucleic acid (e.g., an RNA, e.g., a pre-mRNA), by about 0.5%, 1%, 2%, 3%, 4%, 5%, 6%, 7%, 8%, 9%10%, 15%, 20%, 25%, 30%, 35%, 40%, 45%, 50%, 55%, 60%, 65%, 70%, 75%, 80%, 85%, 90%, 95%, or more, e.g., as determined by qPCR %.

42. A method of forming a complex comprising a component of a spliceosome (e.g., a major spliceosome component or a minor spliceosome component), a nucleic acid (e.g., a DNA, RNA, e.g., a pre-mRNA), and a compound of Formula (I) according to any one of claims 1-35, comprising contacting the nucleic acid (e.g., a DNA, RNA, e.g., a pre-mRNA) with a compound of Formula (I).

43. The method of claim 42, wherein the component of a spliceosome is recruited to the nucleic acid in the presence of the compound of Formula (I).

44. A method of altering the conformation of a nucleic acid (e.g., a DNA, RNA, e.g., a pre-mRNA) comprising contacting the nucleic acid with a compound of Formula (I) according to any one of claims 1-35 or the pharmaceutical composition of claim 36.

45. The method of claim 44, wherein the altering comprises forming a bulge in the nucleic acid.

46. The method of claim 44, wherein the altering comprises stabilizing a bulge in the nucleic acid.

47. The method of claim 44, wherein the altering comprises reducing a bulge in the nucleic acid.

48. The method of any one of any one of claims 44-47, wherein the nucleic acid comprises a splice site.

49. A composition for use in treating a disease or disorder in a subject comprising administering to the subject a compound of Formula (I) according to any one of claims 1-35 or the pharmaceutical composition of claim 36.

50. The composition for use of claim 49, wherein the disease or disorder comprises a proliferative disease (e.g., cancer, a benign neoplasm, or angiogenesis).

51. The composition for use of claim 49, wherein the disease or disorder comprises a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

52. The composition for use of claim 49, wherein the disease or disorder comprises neurological disease or disorder.

53. The composition for use of claim 49, wherein the disease or disorder comprises Huntington's disease.
